(19) United States
(12) Patent Application Publication
(10) Pub. No.: US 2026/0335453 A1
(43) Pub. Date: Oct. 29, 2026

(54) ISOINDOLINONE-GLUTARIMIDE ANTIBODY CONJUGATES, AND USES THEREOF

(71) Applicant: FIREFLY BIO, INC., South San Francisco, CA (US)

(72) Inventors: Luca ARISTA, Riehen (CH); John A. FLYGARE, Burlingame, CA (US); Freya M. HARVEY, Faoug (CH); Thomas RYCKMANS, Kembs (FR); Sean Wesley SMITH, Lake Forest Park, WA (US); Christoph C. GROHMANN, San Francisco, CA (US); Bernhard GEIERSTANGER, Solana Beach, CA (US)

(73) Assignee: FIREFLY BIO, INC., South San Francisco, CA (US)

(21) Appl. No.: 19/483,678

(22) PCT Filed: May 17, 2024
(86) PCT No.: PCT/US2024/029822

§ 371 Date: Nov. 12, 2025

Related U.S. Application Data
(60) Provisional application No. 63/467,543, filed on May 18, 2023.
(60) Provisional application No. 63/557,080, filed on Feb. 23, 2024.

(51) Int. Cl. A61K 47/68; A61P 35/00; C07K 16/28; C07K 16/32

(52) U.S. Cl. CPC A61K 47/6803; A61K 47/6849; A61K 47/6855; A61K 47/6889; A61P 35/00; C07K 16/2803; C07K 16/32

(57) ABSTRACT

The invention provides antibody conjugates of Formula I comprising an antibody linked by conjugation to one or more isoindolinone-glutarimide moieties. The invention also provides isoindolinone-glutarimide derivative intermediate compositions comprising a reactive functional group. Such intermediate compositions are suitable substrates for formation of the antibody conjugates through a linker or linking moiety. The invention further provides methods of treating diseases and disorders such as cancer with the antibody conjugates.

### CROSS REFERENCE TO RELATED APPLICATIONS

This non-provisional application claims the benefit of priority to U.S. Provisional Applications No. 63/467,543, filed 18 May 2023, and No. 63/557,080, filed on 23 Feb. 2024, each of which are incorporated by reference in their entirety.

### FIELD OF THE INVENTION

The disclosure relates generally to antibody conjugate compositions, intermediates for their manufacture, and methods of their use. The compositions are useful for facilitating intracellular degradation of target proteins.

### BACKGROUND OF THE INVENTION

The ubiquitin proteasome system can be manipulated to conduct targeted degradation of specific proteins. Promoting the targeted degradation of pathogenic proteins using small molecule degraders is a new modality in the treatment of diseases, including redirecting the activity of E3 ligases such as cereblon. Proteolysis targeting chimera compounds called PROTAC (Sakamoto, K. M., et al. (2001) Proc. Natl. Acad. Sci. USA 98:8554-8559; Sun, X. et al. (2019) Signal Transduct. Target. Ther. 4:64; Schapira, M., et al (2019) Nat. Rev. Drug Discov. 18:949-963) and “molecular glue” compounds (Yang, Z., et al (2021) Cell Research 31:1315-1318; Tan, X. et al. (2007) Nature 446:640-645; Han, T. et al. (2017) Science 356, eaa13755) are two modes of TPD. PROTAC comprise three parts, including a ligand for binding a target protein, another ligand for recruiting an E3 ligase, and a linker to help anchor the target protein to the E3 ubiquitin ligase to promote its ubiquitination and subsequent proteasomal degradation. Similar to PROTAC, molecular glues can also cause ubiquitination and degradation of a target protein. In contrast to PROTAC, molecular glues are small molecular weight compounds that trigger a compact protein-protein interaction between a target protein and an E3 ubiquitin ligase. Molecular glues are typically smaller than PROTAC and may have better pharmacological properties, higher membrane permeability, better cellular uptake, and better penetration of the blood-brain barrier. Molecular glues promote the poly-ubiquitination and proteasomal degradation of various disease-associated protein targets (Chamberlain, P., et al (2019) Drug Disc. Today: Tech. 31:29-34; WO 2022/152821). The molecular glue molecules bind to both the E3 ligase and the target protein, thereby mediating an alteration of the ligase surface and enabling an interaction with the target protein. Examples include the IMiD (immunomodulatory imide drug) class including thalidomide, lenalidomide and pomalidomide, each approved for use in treating hematological cancers. More efficient targeting strategies are still required.

GSPT1 contains a well-defined degron, a peptidic motif that signals for degradation, allowing for the recruitment of the E3 ligase cereblon (CRBN) and subsequent proteasomal degradation in the presence of molecular glue degraders. GSPT1 (G1 to S phase transition 1) is a translation termination factor that recognizes the termination codon by binding eRF1, forcing the proteins to dissociate from the ribosome after translation. Downregulation of GSPT1 can cause the abnormal expression of key proteins, inhibit proliferation or induce apoptosis in diverse tumor cells (Chauvin, C., et al. (2007) Mol. Cell. Biol. 27:5619-5629; Matyskiela, M. E., et al. (2016) Nature 535:252-257; Yang, J., et al. J. Med. Chem. (2019) 62:9471-9487).

Targeted therapeutic agents to treat hyperproliferative disorders like cancer, and other disease are of interest.

### SUMMARY OF THE INVENTION

The invention is generally directed to an antibody conjugate composition comprising an isoindolinone-glutarimide moiety covalently attached to an antibody by an antibody linker, wherein the antibody binds to a tumor-associated antigen or cell-surface receptor. The antibody conjugate composition has Formula I:

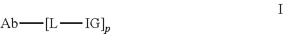

or a pharmaceutically acceptable salt thereof,
wherein:
Ab is the antibody;
L is the antibody linker;
IG is the isoindolinone-glutarimide moiety; and
p is an integer from 1 to 12.

Another aspect of the invention is an antibody conjugate composition selected from Formulae Ia and Ib:

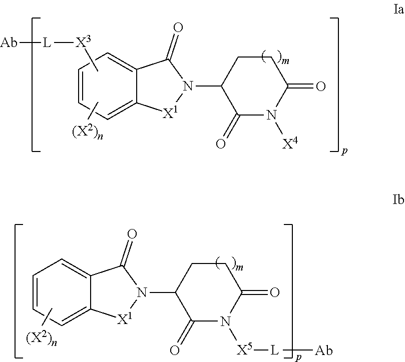

wherein the substituents are defined herein.

Another aspect of the invention is an isoindolinone-glutarimide linker compound selected from Formulae IIa and IIb.

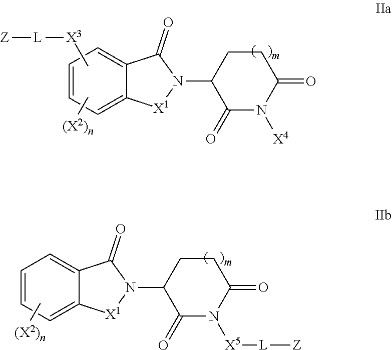

wherein the substituents are defined herein.

Another aspect of the invention is the antibody conjugate composition prepared by conjugation of a cysteine amino acid of an antibody with an isoindolinone-glutarimide linker compound.

Another aspect of the invention is a process for preparing the antibody conjugate comprising reacting a cysteine amino acid of an antibody with an isoindolinone-glutarimide linker compound.

Another aspect of the invention is a pharmaceutical composition comprising a therapeutically effective amount of the antibody conjugate composition, and one or more pharmaceutically acceptable diluent, vehicle, carrier or excipient.

Another aspect of the invention is a method for treating cancer comprising administering a therapeutically effective amount of the pharmaceutical composition to a patient in need thereof,

Another aspect of the invention is a use of the antibody conjugate composition in the manufacture of a medicament for the treatment of cancer in a mammal.

Another aspect of the invention is the antibody conjugate composition for use in a method for treating cancer.

### BRIEF DESCRIPTION OF THE DRAWINGS

FIG. 1 shows a graph of in vivo tumor volume over time in the treatment of CD22+WSU-DLCL2 syngeneic tumor bearing CB17 SCID mice with pinatuzumab, and anti-CD22 conjugates IGAC-59 and IGAC-62 (Table 3).

FIG. 2 shows a graph of in vivo tumor volume over time in the treatment of CD22+WSU-DLCL2 syngeneic tumor bearing CB17 SCID mice with pinatuzumab, and anti-CD22 conjugates IGAC-53, IGAC-60 and IGAC-62 (Table 3).

FIG. 3 shows a graph of in vivo tumor volume over time in the treatment of HER2+NCI-N87 gastric tumor xenograft bearing female BALB/c nude mice with anti-HER2 conjugates IGAC-58 and IGAC-61 (Table 3).

FIG. 4 shows a graph of in vivo tumor volume over time in the treatment of HER2+NCI-N87 gastric tumor xenograft bearing female BALB/c nude mice with anti-HER2 conjugates IGAC-54, IGAC-55, IGAC-56 and IGAC-57 (Table 3).

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### Definitions

The terms “antibody” or “antibody construct” refer to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells.

“Bispecific” antibodies (bsAbs) are antibodies that bind two distinct epitopes to cancer (Suurs F. V. et al (2019) Pharmacology & Therapeutics 201: 103-119). Bispecific antibodies may engage immune cells to destroy tumor cells, deliver isoindolinone-glutarimide moieties to tumors, and/or block tumor signaling pathways. An antibody that targets a particular antigen includes a bispecific or multispecific antibody with at least one antigen binding region that targets the particular antigen. In some embodiments, the targeted monoclonal antibody is a bispecific antibody with at least one antigen binding region that targets tumor cells. Such antigens include but are not limited to: mesothelin, prostate specific membrane antigen (PSMA), HER2, TROP2, CEA, CEACAM5, EGFR, 5T4, Nectin4, CCL-1, CCR7, CD19, CD20, CD22, CD30, CD33, CD70, CD79b, CD123, CDH3, B7H3, B7H4 (also known as 08E), Integrin-beta6, protein tyrosine kinase 7 (PTK7), glypican-3, GPC-1, LIV-1, Folate receptor alpha, Claudin18.2, RG1, fucosyl-GMl, tissue factor (CD142), cKit (CD117), Axl, GC-C, CTLA-4, and CD44 (WO 2017/196598). Other antigen binding regions of bispecific antibodies include those in the following section: ANTIBODY TARGETS.

In some embodiments, the antibody construct is an antigen-binding antibody “fragment,” which comprises at least an antigen-binding region of an antibody, alone or with other components that together constitute the antibody construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain. In some embodiments, the antibody construct is an antibody or a fusion protein comprising (i) an antigen binding domain and (ii) an Fc domain.

The antibody or antibody fragment can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a t-cell receptor, so as to provide a T-cell receptor like construct that binds PD-L1. In yet another embodiment, the antibody fragment is part of a bispecific T-cell engager (BiTEs) comprising a CD1 or CD3 binding domain and linker.

In some embodiments, the antibody construct comprises an Fc domain. In certain embodiments, the antibody construct is a fusion protein. The antigen binding domain can be a single-chain variable region fragment (scFv). A single-chain variable region fragment (scFv), which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology. The antibody construct or antigen binding domain may comprise one or more variable regions (e.g., two variable regions) of an antigen binding domain of an antibody, each variable region comprising a CDR1, a CDR2, and a CDR3.

“Cysteine-mutant antibody” is an antibody in which one or more amino acid residues of an antibody are substituted with cysteine residues. A cysteine-mutant antibody may be prepared from the parent antibody by antibody engineering methods (Junutula, J. et al., (2008b) Nature Biotech., 26(8):925-932; Doman et al. (2009) Blood 114(13):2721-2729; U.S. Pat. Nos. 7,521,541; 7,723,485; US 2012/0121615; WO 2009/052249). Cysteine residues provide for site-specific conjugation of a drug moiety such as a isoindolinone-glutarimide compound to the antibody through the reactive cysteine thiol groups at the engineered cysteine sites but do not perturb immunoglobulin folding and assembly or alter antigen binding and effector functions. Cysteine-mutant antibodies can be conjugated to the isoindolinone-glutarimide linker compound with uniform stoichiometry of the antibody conjugate (e.g., up to two isoindolinone-glutarimide moieties per antibody in an antibody that has a single engineered, mutant cysteine site). The isoindolinone-glutarimide linker compound has a reactive electrophilic group to react specifically with the free cysteine thiol groups of the cysteine-mutant antibody.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

Nucleic acid or amino acid sequence “identity,” as referenced herein, can be determined by comparing a nucleic acid or amino acid sequence of interest to a reference nucleic acid or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100×[(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

The “antibody construct” or “binding agent” comprises Ig heavy and light chain variable region polypeptides that together form the antigen binding site. Each of the heavy and light chain variable regions are polypeptides comprising three complementarity determining regions (CDR1, CDR2, and CDR3) connected by framework regions. The antibody construct can be any of a variety of types of binding agents known in the art that comprise Ig heavy and light chains. For instance, the binding agent can be an antibody, an antigen-binding antibody “fragment,” or a T-cell receptor.

“Amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally-occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

“Linker” refers to a functional group that covalently bonds two or more moieties in an antibody conjugate compound. For example, the linking moiety can serve to covalently bond a drug isoindolinone-glutarimide moiety to an antibody in an antibody conjugate composition. Useful bonds for connecting linking moieties to proteins and other materials include, but are not limited to, amides, amines, esters, carbamates, ureas, thioethers, thiocarbamates, thiocarbonates, and thioureas.

“Divalent” refers to a chemical moiety that contains two points of attachment for linking two functional groups; polyvalent linking moieties can have additional points of attachment for linking further functional groups. Divalent radicals may be denoted by the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, alkoxy, and others.

A wavy line (“ [symbol] ”) and one or more asterisks (*) represents a point of attachment of the specified chemical moiety to another moiety. If the specified chemical moiety has two wavy lines (“ [symbol] ”) present, it will be understood that the chemical moiety can be used bilaterally, i.e., as read from left to right or from right to left. In some embodiments, a specified moiety having two wavy lines (“ [symbol] ”) present is considered to be used as read from left to right.

“Alkyl” refers to a straight (linear) or branched, saturated, aliphatic radical having the number of carbon atoms indicated. Alkyl can include any number of carbons, for example from one to six, one to eight, one to twelve, one to twenty, or one to forty. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

“Alkenyl” refers to a straight (linear) or branched, unsaturated, aliphatic radical having the number of carbon atoms indicated and at least one carbon-carbon double bond, sp2. Alkenyl can include from two to about 12 or more carbons atoms. Alkenyl groups are radicals having “cis” and “trans” orientations, or alternatively, “E” and “Z” orientations. Examples include, but are not limited to, ethylenyl or vinyl (—CH═CH₂), allyl (—CH₂CH═CH₂). butenyl, pentenyl, and isomers thereof. Alkenyl groups can be substituted or unsubstituted. “Substituted alkenyl” groups can be substituted with one or more groups selected from halo, hydroxy, amino, oxo (═O), alkylamino, amido, acyl, nitro, cyano, and alkoxy.

The terms “alkenylene” or “alkenyldiyl” refer to a linear or branched-chain divalent hydrocarbon radical. Examples include, but are not limited to, ethylenylene or vinylene (—CH═CH—), allyl (—CH₂CH═CH—), and the like.

“Alkynyl” refers to a straight (linear) or branched, unsaturated, aliphatic radical having the number of carbon atoms indicated and at least one carbon-carbon triple bond, sp. Alkynyl can include from two to about 12 or more carbons atoms. For example, C₂-C₆ alkynyl includes, but is not limited to ethynyl (—C≡CH), propynyl (propargyl, —CH₂C≡CH), butynyl, pentynyl, hexynyl, and isomers thereof. Alkynyl groups can be substituted or unsubstituted. “Substituted alkynyl” groups can be substituted with one or more groups selected from halo, hydroxy, amino, oxo (═O), alkylamino, amido, acyl, nitro, cyano, and alkoxy.

The term “alkynylene” or “alkynyldiyl” refer to a divalent alkynyl radical.

“Heteroalkyl” or “heteroalkylene” refer to a monovalent, straight or branched chain alkyl group, as defined above, comprising at least one heteroatom including but not limited to Si, N, O, P or S within the alkyl chain or at a terminus of the alkyl chain. In some embodiments, a heteroatom is within the alkyl chain. In other embodiments, a heteroatom is at a terminus of the alkylene and thus serves to join the alkyl to the remainder of the molecule. In some embodiments, a heteroalkyl group may have 1 to 12 carbon atoms (C₁-C₁₂ heteroalkyl). In some embodiments, a heteroalkyl group may have 1 to 24 carbon atoms (C₁-C₂₄ heteroalkyl). In some embodiments, a heteroalkyl group may have 1 to 40 carbon atoms (C₁-C₄₀ heteroalkyl). Unless stated otherwise specifically in the specification, a heteroalkyl group is optionally substituted. For example, heteroalkyl groups can be substituted with 1-6 fluoro (F) substituents, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂CH₂OCH₃, —CH₂CH₂NHCH₃, —CH₂CH₂N(CH₃)₂, —C(═O)NHCH₂CH₂NHCH₃, —C(═O)N(CH₃)CH₂CH₂N(CH₃)₂, —C(═O)NHCH₂CH₂NHC(═O)CH₂CH₃, —C(═O)N(CH₃)CH₂CH₂N(CH₃)C(═O)CH₂CH₃, —OCH₂CH₂CH₂NH(CH₃), —OCH₂CH₂CH₂N(CH₃)₂, —OCH₂CH₂CH₂NHC(═O)CH₂CH₃, —OCH₂CH₂CH₂N(CH₃)C(═O)CH₂CH₃, —CH₂CH₂CH₂NH(CH₃), —OCH₂CH₂CH₂N(CH₃)₂, —CH₂CH₂CH₂NHC(═O)CH₂CH₃, —CH₂CH₂CH₂N(CH₃)C(═O)CH₂CH₃, —CH₂SCH₂CH₃, —CH₂CH₂S(O)CH₃, —NHCH₂CH₂NHC(═O)CH₂CH₃, —CH₂CH₂S(O)₂CH₃, —CH₂CH₂OCF₃, and —Si(CH₃)₃. Up to two heteroatoms may be consecutive, such as, for example, —CH₂NHOCH₃ and —CH₂OSi(CH₃)₃. A terminal polyethylene glycol (PEG) moiety is a type of heteroalkyl group. Exemplary heteroalkyl groups also include ethylene oxide (e.g., polyethylene oxide), propylene oxide, amino acid chains (i.e., short to medium length peptides such as containing 1-15 amino acids), and alkyl chains connected via a variety of functional groups such as amides, disulfides, ketones, phosphonates, phosphates, sulfates, sulfones, sulfonamides, esters, ethers, —S—, carbamates, ureas, thioureas, anhydrides, or the like (including combinations thereof). In some embodiments, a heteroalkyl group includes a polyamino acid having 1-10 amino acids. In some embodiments, a heteroalkyl group includes a polyamino acid having 1-5 amino acids.

Heteroalkyl groups include a solubilizing unit comprising one or more groups of polyglycine, polysarcosine, polyethyleneoxy (PEG), and a glycoside, or combinations thereof.

“Heteroalkenyl” refers to a heteroalkyl group, as defined above, that contains at least one carbon-carbon double bond. “Heteroalkynyl” refers to a heteroalkyl group, as defined above, that contains at least one carbon-carbon triple bond.

“Heteroalkyldiyl” refers to a divalent form of a heteroalkyl group as defined above. In some embodiments, a heteroalkyldiyl group may have 1 to 12 carbon atoms (C₁-C₁₂ heteroalkyldiyl). In some embodiments, a heteroalkyldiyl group may have 1 to 24 carbon atoms (C₁-C₂₄ heteroalkyldiyl). In some embodiments, a heteroalkyldiyl group may have 1 to 40 carbon atoms (C₁-C₄₀ heteroalkyldiyl). Examples of heteroalkyldiyl groups include, but are not limited to, —CH₂CH₂OCH₂—, —CH₂CH₂OCF₂—, —CH₂CH₂NHCH₂—, —CH₂OC(═O)NH—, —CH₂OP(═O)(OH)OCH₂—, C(═O)NHCH₂CH₂NHCH₂—, —C(═O)N(CH₃)CH₂CH₂N(CH₃)CH₂—, —C(═O)NHCH₂CH₂NHC(═O)CH₂CH₂—, —C(═O)N(CH₃)CH₂CH₂N(CH₃)C(═O)CH₂CH₂—, —OCH₂CH₂OCH₂CH₂—, —OCH₂CH₂OCH₂C(═O)—, —OCH₂CH₂OCH₂CH₂C(═O)—, —OCH₂CH₂NHCH₂—, —OCH₂CH₂N(CH₃)CH₂—, —OCH₂CH₂CH₂NHCH₂—, —OCH₂CH₂CH₂N(CH₃)CH₂—, —OCH₂CH₂CH₂NHC(═O)CH₂CH₂—, —OCH₂CH₂CH₂N(CH₃)C(═O)CH₂CH₂—, —CH₂CH₂CH₂NHCH₂—, —CH₂CH₂CH₂N(CH₃)CH₂—, —CH₂CH₂CH₂NHC(═O)CH₂CH₂—, —CH₂CH₂CH₂N(CH₃)C(═O)CH₂CH₂—, —CH₂CH₂NHC(═O)—, —CH₂CH₂N(CH₃)CH₂—, —CH₂CH₂N⁺(CH₃)₂—, —NHCH₂CH₂(NH₂)CH₂—, and —NHCH₂CH₂(NHCH₃)CH₂—. A divalent polyethylene glycol (PEG) moiety with one to about 50 units of —OCH₂CH₂— is a type of heteroalkyldiyl group. “Heteroalkenyldiyl” refers to a divalent form of a heteroalkenyl group. “Heteroalkynyldiyl” refers to a divalent form of a heteroalkynyl group.

The terms “carbocycle”, “carbocyclyl”, “carbocyclic ring” and “cycloalkyl” refer to a saturated or partially unsaturated, monocyclic, fused bicyclic, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “cycloalkyldiyl” refers to a divalent cycloalkyl radical.

“Aryl” refers to a monovalent aromatic hydrocarbon radical of 6-20 carbon atoms (C₆-C₂₀) derived by the removal of one hydrogen atom from a single carbon atom of a parent aromatic ring system. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The terms “arylene” or “aryldiyl” mean a divalent aromatic hydrocarbon radical of 6-20 carbon atoms (C₆-C₂M) derived by the removal of two hydrogen atom from a two carbon atoms of a parent aromatic ring system. Some aryldiyl groups are represented in the exemplary structures as “Ar”. Aryldiyl includes bicyclic radicals comprising an aromatic ring fused to a saturated, partially unsaturated ring, or aromatic carbocyclic ring. Typical aryldiyl groups include, but are not limited to, radicals derived from benzene (phenyldiyl), substituted benzenes, naphthalene, anthracene, biphenylene, indenylene, indanylene, 1,2-dihydronaphthalene, 1,2,3,4-tetrahydronaphthyl, and the like. Aryldiyl groups are also referred to as “arylene”, and are optionally substituted with one or more substituents described herein.

The terms “heterocycle,” “heterocyclyl” and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo[4,5], [5,5], [5,6], or [6,6] system. Heterocycles are described in Paquette, Leo A.; “Principles of Modern Heterocyclic Chemistry” (WA. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro heterocyclyl moieties are also included within the scope of this definition. Examples of spiro heterocyclyl moieties include azaspiro[2.5]octanyl and azaspiro[2.4]heptanyl. Examples of a heterocyclic group wherein 2 ring atoms are substituted with oxo (═O) moieties are pyrimidinonyl and 1,1-dioxo-thiomorpholinyl. The heterocycle groups herein are optionally substituted independently with one or more substituents described herein.

The term “heterocyclyldiyl” refers to a divalent, saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents as described. Examples of 5-membered and 6-membered heterocyclyldiyls include morpholinyldiyl, piperidinyldiyl, piperazinyldiyl, pyrrolidinyldiyl, dioxanyldiyl, thiomorpholinyldiyl, and S-dioxothiomorpholinyldiyl.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The term “heteroaryldiyl” refers to a divalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of 5-membered and 6-membered heteroaryldiyls include pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindolinone, position 4 of a morpholine, and position 9 of a carbazole, or p-carboline.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

As used herein, the phrase “quaternary ammonium salt” refers to a tertiary amine that has been quaternized with an alkyl substituent (e.g., a C₁-C₄ alkyl such as methyl, ethyl, propyl, or butyl).

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

The phrases “effective amount” and “therapeutically effective amount” refer to a dose or amount of a substance such as the antibody conjugate of the invention that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the antibody conjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the antibody conjugate may prevent growth and/or kill existing cancer cells, it may be cytostatic and/or cytotoxic. For cancer therapy, efficacy can, for example, be measured by assessing the time to disease progression (TTP) and/or determining the response rate (RR)

“Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

### Antibodies

The antibody conjugate compositions of the invention comprise an antibody. Included in the scope of the embodiments of the invention are functional variants of the antibody constructs or antigen binding domain described herein. The term “functional variant” as used herein refers to an antibody construct having an antigen binding domain with substantial or significant sequence identity or similarity to a parent antibody construct or antigen binding domain, which functional variant retains the biological activity of the antibody construct or antigen binding domain of which it is a variant. Functional variants encompass, for example, those variants of the antibody constructs or antigen binding domain described herein (the parent antibody construct or antigen binding domain) that retain the ability to recognize target cells expressing a tumor-associated antigen or cell surface receptor to a similar extent, the same extent, or to a higher extent, as the parent antibody construct or antigen binding domain.

In reference to the antibody construct or antigen binding domain, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the antibody construct or antigen binding domain.

A functional variant can, for example, comprise the amino acid sequence of the parent antibody construct or antigen binding domain with at least one conservative amino acid substitution. Alternatively, or additionally, the functional variants can comprise the amino acid sequence of the parent antibody construct or antigen binding domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody construct or antigen binding domain.

A functional variant can, for example, comprise the amino acid sequence of the parent antibody construct or antigen binding with at least one non-canonical amino acid (ncAA) substitution (L Wang, et al, (2001) Science, 292(5516):498-500, CC Liu, PG Schultz, (2010) Annu Rev Biochem. 79:413-44).

The antibodies comprising the antibody conjugate compositions of the invention include Fc engineered variants. In some embodiments, the mutations in the Fc region that result in modulated binding to one or more Fc receptors can include one or more of the following mutations: YTE (M252Y/S254T/T256E), LALAPA (L234A/L235A/P329A), SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E345R, E345R/E430G, E345K, E233, G237, P238, H268, P271, L328 and A330. Additional Fc region modifications for modulating Fc receptor binding are described in, for example, US 2016/0145350, U.S. Pat. Nos. 7,416,726 and 5,624,821, which are hereby incorporated by reference in their entireties herein.

The antibodies comprising the antibody conjugate compositions of the invention include glycan variants, such as afucosylation. In some embodiments, the Fc region of the binding agents are modified to have an altered glycosylation pattern of the Fc region compared to the native non-modified Fc region.

In some embodiments, the antibodies in the antibody conjugate compositions contain a modified Fc region, wherein the modification modulates the binding of the Fc region to one or more Fc receptors.

In some embodiments, the antibodies in the antibody conjugate contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the antibody conjugate compositions contain one or more modifications (e.g., amino acid insertion, deletion, and/or substitution) in the Fc region that reduce the binding of the Fc region of the antibody to FcγRIIB. In some embodiments, the antibodies in the antibody conjugate compositions contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the antibody conjugate compositions contain one of more modifications in the Fc region that increase the binding of the Fc region of the antibody to FcγRIIB.

In some embodiments, the modulated binding is provided by mutations in the Fc region of the antibody relative to the native Fc region of the antibody. The mutations can be in a CH₂ domain, a CH₃ domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., cetuximab). Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fcs (Jefferis et al., (2009) mAbs, 1(4):332-338).

In some embodiments, the Fc region of the antibodies of the antibody conjugate compositions are modified to have an altered glycosylation pattern of the Fc region compared to the native non-modified Fc region. Human immunoglobulin is glycosylated at the Asn297 residue in the Cγ2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while afucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726, US 2007/0014795 and US 2008/0286819, which are hereby incorporated by reference in their entireties.

In some embodiments, the antibodies of the antibody conjugate compositions are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the antibody conjugate compositions are engineered to be afucosylated or glycosylated.

In some embodiments, the antibodies in the antibody conjugate compositions are a cysteine-engineered antibody which provides for site-specific conjugation of an adjuvant, label, or drug moiety to the antibody through cysteine substitutions at sites where the engineered cysteines are available for conjugation but do not perturb immunoglobulin folding and assembly or alter antigen binding and effector functions (Junutula, et al., (2008) Nature Biotech., 26(8):925-932; Dornan et al. (2009) Blood 114(13):2721-2729; U.S. Pat. Nos. 7,521,541; 7,723,485; US 2012/0121615; WO 2009/052249). A “cysteine engineered antibody” or “cysteine engineered antibody variant” is an antibody in which one or more residues of an antibody are substituted with cysteine residues. Cysteine-engineered antibodies can be conjugated to the isoindolinone-glutarimide (IG) moiety with uniform stoichiometry (e.g., up to two IG moieties per antibody in an antibody that has a single engineered cysteine site).

In some embodiments, cysteine-engineered antibodies are used to prepare antibody conjugate compositions with a reactive cysteine thiol residue introduced at a site on the light chain, such as the 149-lysine site (LC K149C), or on the heavy chain such as the 122-serine site (HC S122C), as numbered by Kabat numbering. In other embodiments, the cysteine-engineered antibodies have a cysteine residue introduced at the 375-serine site (EU numbering) of the heavy chain (HC S375C). In other embodiments, the cysteine-engineered antibodies have a cysteine residue introduced at the 118-alanine site (EU numbering) of the heavy chain (HC A118C). This site is alternatively numbered 121 by Sequential numbering or 114 by Kabat numbering. In other embodiments, the cysteine-engineered antibodies have a cysteine residue introduced in: (i) the light chain at G64C, R142C, K188C, L201C, T129C, S114C, or E105C according to Kabat numbering; (ii) the heavy chain at D101C, V184C, T205C, or S122C according to Kabat numbering; or (iii) other cysteine-mutant antibodies, and as described in Bhakta, S. et al, (2013) “Engineering THIOMABs for Site-Specific Conjugation of Thiol-Reactive Linkers”, Laurent Ducry (ed.), Antibody-Drug Conjugates, Methods in Molecular Biology, vol. 1045, pages 189-203; WO 2011/156328; U.S. Pat. No. 9,000,130.

In some embodiments, the antibody is a full-length antibody. In certain embodiments, the antibody is an antigen binding fragment. In some embodiments, the antibody is a humanized antibody.

In some embodiments, the antibody is an anti-CD40 antibody, an antibody selected from an anti-LRRC15 antibody, an anti-CTSK antibody, an anti-ADAM12 antibody, an anti-ITGA11 antibody, an anti-FAP antibody, an anti-NOX4 antibody, an anti-SGCD antibody, an anti-SYNDIG1 antibody, an anti-CDH11 antibody, an anti-PLPP4 antibody, an anti-SLC24A2 antibody, an anti-PDGFRB antibody, an anti-THY1 antibody, an anti-ANTXR1 antibody, an anti-GAS1 antibody, an anti-CALHM5 antibody, an anti-SDC1 antibody, an anti-HER2 antibody, an anti-TROP2 antibody, an anti-MSLN antibody, an anti-Nectin4 antibody, an anti-ASGR1 antibody, and an anti-MUC16 antibody.

In some embodiments, the antibody or Fc fusion protein is selected from: abagovomab, abatacept (also known as ORENCIA®), abciximab (also known as REOPRO®), c7E3 Fab), adalimumab (also known as HUMIRA®), adecatumumab, alemtuzumab (also known as CAMPATH®), MabCampath or Campath-1H), altumomab, afelimomab, panitumumab, mafenatox, anrukizumab, apolizumab, arcitumomab, aselizumab, atlizumab, atorolimumab, bapineuzumab, basiliximab (also known as SIMULECT®), bavituximab, bectumomab (also known as LYMPHOSCAN®), belimumab (also known as LYMPHO-STAT-B®), bertilimumab, besilesomab, bevacizumab (also known as AVASTIN®), biciromab brallobarbital, bivatuzumab mertansine, campath, canakinumab (also known as ACZ885), cantuzumab mertansine, capromab (also known as PROSTASCINT®), catumaxomab (also known as REMOVAB®), cedelizumab (also known as CIMZIA®), certolizumab pegol, cetuximab (also known as ERBITUX®), clenoliximab, dacetuzumab, dacliximab, daclizumab (also known as ZENAPAX®), datopotamab, denosumab (also known as AMG 162), detumomab, dorlimomab aritox, dorlixizumab, duntumumab, durimulumab, durmulumab, ecromeximab, eculizumab (also known as SOLIRIS®), edobacomab, edrecolomab (also known as Mab17-1A, PANOREX®), efalizumab (also known as RAPTIVA®), efungumab (also known as MYCOGRAB®), elsilimomab, enapotamab, enfortumab, enlimomab pegol, epitumomab cituxetan, efalizumab, epitumomab, epratuzumab, erlizumab, ertumaxomab (also known as REXOMUN®), etanercept (also known as ENBREL®), etaracizumab (also known as etaratuzumab, VITAXIN®, ABEGRIN®), exbivirumab, fanolesomab (also known as NEUTROSPEC®), faralimomab, felvizumab, fontolizumab (also known as HUZAF®), galiximab, gantenerumab, gavilimomab (also known as ABXCBL®), gemtuzumab ozogamicin (also known as MYLOTARG®), golimumab (also known as CNTO 148), gomiliximab, ibalizumab (also known as TNX-355), ibritumomab tiuxetan (also known as ZEVALIN®), igovomab, imciromab, indusatumab, infliximab (also known as REMICADE®), inolimomab, inotuzumab ozogamicin, ipilimumab (also known as MDX-010, MDX-101), iratumumab, keliximab, labetuzumab, ladiratuzumab, lemalesomab, lebrilizumab, lerdelimumab, lexatumumab (also known as, HGS-ETR2, ETR2-STO1), lexitumumab, libivirumab, lintuzumab, loncastuximab, losatuxizumab, lucatumumab, lumiliximab, mapatumumab (also known as HGSETR1, TRM-1), maslimomab, matuzumab (also known as EMD72000), mepolizumab (also known as BOSATRIA®), metelimumab, milatuzumab, miltuximab, minretumomab, mirvetuximab, mitumomab, morolimumab, motavizwnab (also known as NUMAX®), muromonab (also known as OKT3), nacolomab tafenatox, naptumomab estafenatox, natalizumab (also known as TYSABRI®, ANTEGREN®), nebacumab, nerelimomab, nimotuzumab (also known as THERACIM hR3®, THERA-CIM-hR3®, THERALOC®), nofetumomab merpentan (also known as VERLUMA®), ocrelizumab, odulimomab, ofatumumab, omalizumab (also known as XOLAIR®), oregovomab (also known as OVAREX®), otelixizumab, pagibaximab, palivizumab (also known as SYNAGIS®), panitumumab (also known as ABX-EGF, VECTIBIX®), pascolizumab, pemtumomab (also known as THERAGYN®), pertuzumab (also known as 2C4, OMNITARG®), pexelizumab, pinatuzumab, pintumomab, polatuzumab, priliximab, pritumumab, ranibizumab (also known as LUCENTIS®), raxibacumab, regavirumab, reslizumab, rituximab (also known as RITUXAN®, MabTHERA®), rovelizumab, ruplizumab, Sacituzumab, satumomab, sevirumab, sibrotuzumab, siplizumab (also known as MEDI-507), sontuzumab, stamulumab (also known as MYO-029), sulesomab (also known as LEUKOSCAN®), tacatuzumab tetraxetan, tadocizumab, talizumab, taplitumomab paptox, tefibazumab (also known as AUREXIS®), telimomab aritox, teneliximab, teplizumab, ticilimumab, tocilizumab (also known as ACTEMRA®), tisotumab, toralizumab, tositumomab, trastuzumab (also known as HERCEPTIN®), tremelimumab (also known as CP-675,206), tucotuzumab celmoleukin, tuvirumab, urtoxazumab, ustekinumab (also known as CNTO 1275), vapaliximab, veltuzumab, vepalimomab, visilizumab (also known as NUVION®), volociximab (also known as M200), votumumab (also known as HUMASPECT®), zalutumumab, zanolimumab (also known as HuMAX-CD4), ziralimumab, zolimomab aritox, daratumumab, elotuxumab, obintunzumab, olaratumab, brentuximab vedotin, afibercept, abatacept, belatacept, afibercept, etanercept, romiplostim, SBT-040 (sequences listed in US 2017/0158772. In some embodiments, the antibody is rituximab.

In an exemplary embodiment, the antibody conjugate composition of the invention comprises an antibody construct that comprises an antigen binding domain that specifically recognizes and binds HER2. In certain embodiments, the antibody conjugate composition comprises an anti-HER2 antibody. In one embodiment of the invention, an anti-HER2 antibody of an antibody conjugate composition of the invention comprises a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of U.S. Pat. No. 5,821,337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN™ (Genentech, Inc.). Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; Slamon et al (2001) New Engl. J. Med. 344:783-792).

In an embodiment of the invention, the antibody construct or antigen binding domain comprises the CDR regions of trastuzumab. In an embodiment of the invention, the anti-HER2 antibody further comprises the framework regions of the trastuzumab. In an embodiment of the invention, the anti-HER2 antibody further comprises one or both variable regions of trastuzumab.

In another embodiment of the invention, an anti-HER2 antibody of an antibody conjugate composition of the invention comprises a humanized anti-HER2 antibody, e.g., humanized 2C4, as described in U.S. Pat. No. 7,862,817. An exemplary humanized 2C4 antibody is pertuzumab (CAS Reg. No. 380610-27-5), PERJETA™ (Genentech, Inc.). Pertuzumab is a HER dimerization inhibitor (HDI) and functions to inhibit the ability of HER2 to form active heterodimers or homodimers with other HER receptors (such as EGFR/HER1, HER2, HER3 and HER4). See, for example, Harari and Yarden, Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003). PERJETA™ is approved for the treatment of breast cancer.

In an embodiment of the invention, the antibody construct or antigen binding domain comprises the CDR regions of pertuzumab. In an embodiment of the invention, the anti-HER2 antibody further comprises the framework regions of the pertuzumab. In an embodiment of the invention, the anti-HER2 antibody further comprises one or both variable regions of pertuzumab.

In an exemplary embodiment, the antibody conjugate composition of the invention comprises an antibody construct that comprises an antigen binding domain that specifically recognizes and binds the B-cell receptor CD22. In certain embodiments, the antibody conjugate composition comprises an anti-CD22 antibody. In one embodiment of the invention, the anti-CD22 antibody of an antibody conjugate composition is pinatuzumab (CAS Reg. No. 1639820-81-7), with complementarity determining regions (CDRs), heavy chain (HC), and light chain (LC) as described in U.S. Pat. No. 8,226,945 and WO 2007/140371 which are incorporated by reference herein.

### Antibody Targets

In some embodiments, the antibody of an antibody conjugate composition is capable of binding one or more antigen targets selected from (e.g., specifically binds to a target selected from) 5T4, ABL, ABCF1, ACVR1, ACVR1B, ACVR2, ACVR2B, ACVRL1, ADORA2A, Aggrecan, AGR2, AICDA, AIF1, AIGI, AKAP1, AKAP2, AMH, AMHIR2, ANGPT1, ANGPT2, ANGPTL3, ANGPTL4, ANPEP, APC, APOC1, AR, aromatase, ATX, AX1, Axl, AZGP1 (zinc-a-glycoprotein), B7.1, B7.2, B7-H1, B7-H3, B7-H4, BAD, BAFF, BAG1, BAI1, BCR, BCL2, BCL6, BDNF, BLNK, BLR1 (MDR15), BIyS, BMP1, BMP2, BMP3B (GDFIO), BMP4, BMP6, BMP8, BMPRTA, BMPR1B, BMPR2, BPAG1 (plectin), BRCA1, C19orflO (IL27w), C3, C4A, C5, C5R1, CANT1, CAPRIN-1, CASPI, CASP4, CAV1, CCBP2 (D6/JAB61), CCLI (1-309), CCLI1 (eotaxin), CCL13 (MCP-4), CCL15 (MIP-Id), CCL16 (HCC-4), CCL17 (TARC), CCL18 (PARC), CCL19 (MIP-3b), CCL2 (MCP-1), MCAF, CCL20 (MIP-3a), CCL21 (MEP-2), SLC, exodus-2, CCL22(MDC/STC-1), CCL23 (MPIF-I), CCL24 (MPIF-2/eotaxin-2), CCL25 (TECK), CCL26 (eotaxin-3), CCL27 (CTACK/ILC), CCL28, CCL3 (MIP-Ia), CCL4 (MIPIb), CCL5 (RANTES), CCL7 (MCP-3), CCL8 (mcp-2), CCNA1, CCNA2, CCND1, CCNE1, CCNE2, CCR1 (CKR1/HM145), CCR2 (mcp-IRB/RA), CCR3 (CKR3/CMKBR3), CCR4, CCR5 (CMKBR5/ChemR13), CCR6 (CMKBR6/CKR-L3/STRL22/DRY6), CCR7 (CKR7/EBI1), CCR8 (CMKBR8/TERI/CKR-L1), CCR9 (GPR-9-6), CCRL1 (VSHK1), CCRL2 (L-CCR), CD164, CD19, CDIC, CD2, CD20, CD21, CD200, CD22, CD24, CD27, CD28, CD3, CD30, CD33, CD35, CD37, CD38, CD3E, CD3G, CD3Z, CD4, CD38, CD40, CD40L, CD44, CD45RB, CD47, CD52, CD69, CD72, CD74, CD79A, CD79B, CD8, CD80, CD81, CD83, CD86, CD117 (cKit), CD123, CD137, CD152, CD274, CDH1 (Ecadherin), CDH3 (Pcadherin), CDH10, CDH12, CDH13, CDH18, CDH19, CDH2O, CDH5, CDH7, CDH8, CDH9, CDK2, CDK3, CDK4, CDK5, CDK6, CDK7, CDK9, CDKN1A (p21Wap1/Cip1), CDKN1B (p27Kip1), CDKNIC, CDKN2A (p16INK4a), CDKN2B, CDKN2C, CDKN3, CEACAM5, CEACAM6, CEBPB, CERI, CHGA, CHGB, Chitinase, CHST10, CKLFSF2, CKLFSF3, CKLFSF4, CKLFSF5, CKLFSF6, CKLFSF7, CKLFSF8, CLDN3, CLDN7 (claudin-7), CLDN18.2 (claudin 18.2), CLL-1, CLN3, CLU (clusterin), cMet, CMKLR1, CMKOR1 (RDC1), CNR1, COL18A1, COLIA1, COL4A3, COL6A1, CR2, Cripto, CRP, CSF1 (M-CSF), CSF2 (GM-CSF), CSF3 (GCSF), CTL8, CTNNB1 (b-catenin), CTSB (cathepsin B), CX3CL1 (SCYD1), CX3CR1 (V28), CXCL1 (GRO1), CXCL10 (IP-IO), CXCLI1 (1-TAC/IP-9), CXCL12 (SDF1), CXCL13, CXCL14, CXCL16, CXCL2 (GRO2), CXCL3 (GRO3), CXCL5 (ENA-78/LIX), CXCL6 (GCP-2), CXCL9 (MIG), CXCR3 (GPR9/CKR-L2), CXCR4, CXCR6 (TYMSTR/STRL33/Bonzo), CYB5, CYC1, CYSLTR1, DAB2IP, DES, DKFZp45IJ0118, DNCL1, DPP4, E2F1, Engel, Edge, Fennel, EFNA3, EFNB2, EGF, EGFR, ELAC2, ENG, Enola, ENO2, ENO3, EPHA1, EPHA2, EPHA3, EPHA4, EPHA5, EPHA6, EPHA7, EPHA8, EPHA9, EPRA10, EPHB1, EPHB2, EPHB3, EPHB4, EPHB5, EPHB6, EPHRIN-A1, EPHRIN-A2, EPHRINA3, EPHRIN-A4, EPHRIN-A5, EPHRIN-A6, EPHRIN-B1, EPHRIN-B2, EPHRIN-B3, EPHB4, EPG, ERBB2 (Her-2), EREG, ERK8, Estrogen receptor, Earl, ESR2, F3 (TF), FADD, farnesyltransferase, FasL, FAP, FASNf, FCER1A, FCER2, FCGR3A, FGF, FGF1 (aFGF), FGF10, FGF11, FGF12, FGF12B, FGF13, FGF14, FGF16, FGF17, FGF18, FGF19, FGF2 (bFGF). FGF20, FGF21, FGF22, FGF23, FGF3 (int-2), FGF4 (HST), FGF5, FGF6 (HST-2), FGF7 (KGF), FGF8, FGF9, FGFR3, FIGF (VEGFD), FILI (EPSILON), FBL1 (ZETA), FLJ12584, FLJ25530, FLRT1 (fibronectin), FLTJ, FLT-3, FOLR1 (folate receptor alpha), FOS, FOSL1 (FRA-1), FY (DARC), GABRP (GABAa), GAGEB1, GAGEC1, GALNAC4S-6ST, GATA3, GD2, GDF5, GFI1, GGT1, GM-CSF, GNAS1, GNRH1, GPC-1, GPR2 (CCR10), GPR31, GPR44, GPR81 (FKSG80), GRCC1O (C1O), GRP, GSN (Gelsolin), GSTP1, HAVCR2, HDAC, HDAC4, HDAC5, HDAC7A, HDAC9, Hedgehog, HGF, HIF1A, HIP1, histamine and histamine receptors, HLA-A, HLA-DRA, HLA-E, HM74, HMOXI, HSP90, HUMCYT2A, ICEBERG, ICOSL, ID2, IFN-a, IFNA1, IFNA2, IFNA4, IFNA5, EFNA6, BFNA7, IFNB1, IFNgamma, IFNW1, IGBP1, IGF1, IGFIR, IGF2, IGFBP2, IGFBP3, IGFBP6, DL-1, ILIO, ILIORA, ILIORB, IL-1, IL1R1 (CD121a), IL1R2 (CD121b), IL-IRA, IL-2, IL2RA (CD25), IL2RB (CD122), IL2RG (CD132), IL-4, IL-4R (CD123), IL-5, IL5RA (CD125), IL3RB (CD131), IL-6, IL6RA, (CD126), IR6RB (CD130), IL-7, IL7RA (CD127), IL-8, CXCR1 (IL8RA), CXCR2, (IL8RB/CD128), IL-9, IL9R (CD129), IL-10, IL10RA (CD210), IL10RB (CDW210B), IL-11, IL11RA, IL-12, IL-12A, IL-12B, IL-12RB1, IL-12RB2, IL-13, IL13RA1, IL13RA2, IL14, IL15, IL15RA, IL16, IL17, IL17A, IL17B, IL17C, IL17R, IL18, IL18BP, IL18R1, ILT8RAP, IL19, ILIA, ILIB, ILIF10, ILIF5, IL1F6, ILIF7, IL1F8, DL1F9, ILIHYI, ILIR1, ILIR2, ILIRAP, ILIRAPLI, ILIRAPL2, ILIRL1, IL1RL2, ILIRN, IL2, IL20, IL20RA, IL21R, IL22, IL22R, IL22RA2, IL23, DL24, IL25, IL26, IL27, IL28A, IL28B, IL29, IL2RA, IL2RB, IL2RG, IL3, IL30, IL3RA, IL4, IL4, IL6ST (glycoprotein 130), ILK, INHA, INHBA, INSL3, INSL4, IRAK1, IRAK2, ITGA1, ITGA2, ITGA3, ITGA6 (.alpha.6 integrin), ITGAV, ITGB3, ITGB4 (.beta.4 integrin), ITGB6 (beta.6 integrin), JAG1, JAK1, JAK3, JTB, JUN, K6HF, KAI1, KDR, KITLG, KLF5 (GC Box BP), KLF6, KLK10, KLK12, KLK13, KLK14, KLK15, KLK3, KLK4, KLK5, KLK6, KLK9, KRT1, KRT19 (Keratin 19), KRT2A, KRTHB6 (hair-specific type II keratin), LAMA5, LEP (leptin), Lingo-p75, Lingo-Troy, LIV-1, LPS, LRRC15, LTA (TNF-b)), LTB, LTB4R (GPR16), LTB4R2, LTBR, MACMARCKS, MAG or OMgp, MAP2K7 (c-Jun), MCP-1, MDK, MIB1, midkine, MIF, MISRII, MJP-2, MK, M1K67 (Ki-67), MMP2, MMP9, MS4AJ, MSMB, MT3 (metallothionectin-UI), mTOR, MTSS1, MUC1 (mucin), MUC16, MYC, MYD88, NCK2, neurocan, Nectin-4, NFKBI, NFKB2, NGFB (NGF), NGFR, NgR-Lingo, NgRNogo66, (Nogo), NgR-p75, NgR-Troy, NMEI (NM23A), NOTCH, NOTCH1, NOX5, NPPB, NROB1, NROB2, NRID1, NR1D2, NR1H2, NR1H3, NR1H4, NR112, NR113, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRP1, NRP2, NTSE, NTN4, ODZI, OPRDI, P2RX7, PAP, PARTI, PATE, PAWR, PCA3, PCDGF, PCNA, PDGFA, PDGFB, PDGFRA, PDGFRB, PECAMI, peg-asparaginase, PF4 (CXCL4), PGF, PGR, phosphacan, PIAS2, PI3 Kinase, PIK3CG, PLAU (uPA), PLG, PLXDCI, PKC, PKC-beta, PPBP (CXCL7), PPID, PR1, PRKCQ, PRKD1, PRL, PROC, PROK2, PSAP, PSCA, PSMA, PTAFR, PTEN, PTGS2 (COX-2), PIN, RAC2 (P21Rac2), RANK, RANK ligand, RARB, RGS1, RGS13, RGS3, RNFI1O (ZNF144), Ron, ROBO2, RXR, S100A2, SCGB 1D2 (lipophilin B), SCGB2A1 (mammaglobin 2), SCGB2A2 (mammaglobin 1), SCYE1 (endothelial Monocyte-activating cytokine), SDF2, SERPENA1, SERPINA3, SERPINB5 (maspin), SERPINEI (PAI-I), SERPINFI, SHIP-1, SHIP-2, SHB1, SHB2, SHBG, SfcAZ, SLC2A2, SLC33A1, SLC43A1, SLIT2, SPPJ, SPRR1B (Spr1), ST6GAL1, STAB1, STATE, STEAP, STEAP2, TB4R2, TBX21, TCP1O, TDGF1, TEK, TGFA, TGFB1, TGFB1I1, TGFB2, TGFB3, TGFBI, TGEBR1, TGFBR2, TGFBR3, THIL, THBS1 (thrombospondin-1), THBS2, THBS4, THPO, TIE (Tie-1), TIMP3, tissue factor, TLR1, TLR2, TLR3, TLR4, TLR5, TLR6, TLR7, TLR8, TLR9, TLR10, TLR11, TNF, TNF-a, TNFAIP2 (B94), TNFAIP3, TNFRSF11A, TNFRSF1A, TNFRSF1B, TNFRSF21, TNFRSF5, TNFRSF6 (Fas), TNFRSF7, TNFRSF8, TNFRSF9, TNFSF10 (TRAIL), TNFSF1T (TRANCE), TNFSF12 (APO3L), TNFSF13 (April), TNFSF13B, TNSF14 (HVEM-L), TNFRSF14 (HVEM), TNFSF15 (VEGI), TNFSF18, TNFSF4 (OX40 ligand), TNFSF5 (CD40 ligand). TNFSF6 (FasL), TNFSF7 (CD27 ligand), TNFSF8 (CD30 ligand), TNFSF9 (4-1BB ligand), TOLLIP, Toll-like receptors, TOP2A (topoisomerase 1ia), TP53, TPM1, TPM2, TRADD, TRAF1, TRAF2, TRAF3, TRAF4, TRAF5, TRAF6, TRKA, TREM1, TREM2, TROP2, TRPC6, TSLP, TWEAK, Tyrosinase, uPAR, VEGF, VEGFB, VEGFC, versican, VHL C5, VLA-4, Wnt-1, XCL1 (tymphotactin), XCL2 (SCM-Ib), XCRI (GPR5/CCXCRJ), YYI, ZFPM2, CLEC4C (BDCA-2, DLEC, CD303, CLECSF7), CLEC4D (MCL, CLECSF8), CLEC4E (Mincle), CLEC6A (Dectin-2). CLEC5A (MDL-1, CLECSF5), CLEC1B (CLEC-2), CLEC9A (DNGR-1), CLEC7A (Dectin-1), PDGFRa, SLAMF7, GP6 (GPVI), LILRA1 (CD851), LILRA2 (CD85H, ILT1), LILRA4 (CD85G, ILT7), LILRA5 (CD85F, ILT11), LILRA6 (CD85b, ILT8), NCR1 (CD335, LY94, NKp46), NCR3 (CD335, LY94, NKp46), NCR3 (CD337, NKp30), OSCAR, TARM1, CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44), PILRB, SIGLEC1 (CD169, SN), SIGLEC14, SIGLEC15 (CD33L3), SIGLEC16, SIRPB1 (CD172B), TREM1 (CD354), Tissue Factor (CD142), TREM2, and KLRF1 (NKp80).

In some embodiments, the antibody binds to an antigen selected from CDH1, CD19, CD20, CD29, CD30, CD38, CD40, CD47, EpCAM, MUC1, MUC16, EGFR, HER2, SLAMF7, and gp75.

In some embodiments, the antibody of an antibody conjugate composition of the invention is capable of binding to one or more tumor-associated antigens (TAA), cell-surface receptors, and immune-specific antigens to confer specificity to the targeting of the conjugate and enable safe and systemic delivery of an active drug moiety.

Certain tumor-associated antigens are known in the art, and can be prepared for use in generating antibodies using methods and information which are well known in the art. In attempts to discover effective cellular targets for cancer diagnosis and therapy, researchers have sought to identify transmembrane or otherwise tumor-associated polypeptides that are specifically expressed on the surface of one or more particular type(s) of cancer cell as compared to on one or more normal non-cancerous cell(s). Often, such tumor-associated polypeptides are more abundantly expressed on the surface of the cancer cells as compared to on the surface of the non-cancerous cells. The identification of such tumor-associated cell surface antigen polypeptides allows more specificity in targeting cancer cells for destruction via antibody-based therapies.

Examples of TAAs include, but are not limited to, those listed below including (1)-(54). For convenience, information relating to these antigens, all of which are known in the art, is listed below and includes names, alternative names, Genbank accession numbers and primary reference(s), following nucleic acid and protein sequence identification conventions of the National Center for Biotechnology Information (NCBI). Nucleic acid and protein sequences corresponding to TAAs listed below including (1)-(54) are available in public databases such as GenBank. TAAs targeted by antibodies include all amino acid sequence variants and isoforms possessing at least about 70%, 80%, 85%, 90%, or 95% sequence identity relative to the sequences identified in the cited references, and/or which exhibit substantially the same biological properties or characteristics as a TAA having a sequence found in the cited references. For example, a TAA having a variant sequence generally is able to bind specifically to an antibody that binds specifically to the TAA with the corresponding sequence listed. The sequences and disclosure in the reference specifically recited herein are expressly incorporated by reference.

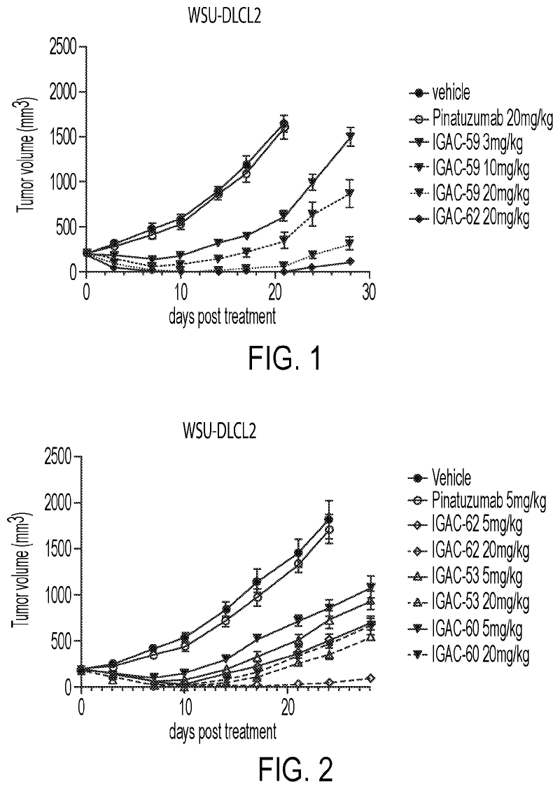

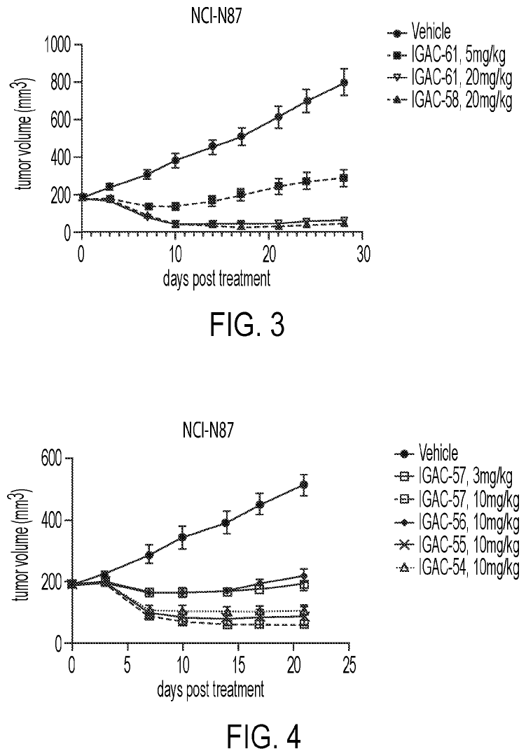

The disclosure in the references specifically recited herein are expressly incorporated by reference.
(1) BMPR1B (bone morphogenetic protein receptor-type IB, Genbank accession no. NM_001203) ten Dijke, P., et al. Science 264 (5155):101-104 (1994), Oncogene 14 (11):1377-1382 (1997)); WO2004063362 (Claim 2); WO2003042661 (Claim 12); US2003134790-A1 (Page 38-39); WO2002102235 (Claim 13; Page 296); WO2003055443 (Page 91-92); WO200299122 (Example 2; Page 528-530); WO2003029421 (Claim 6); WO2003024392 (Claim 2; FIG. 112); WO200298358 (Claim 1; Page 183); WO200254940 (Page 100-101); WO200259377 (Page 349-350); WO200230268 (Claim 27; Page 376); WO200148204 (Example; FIG. 4) NP_001194 bone morphogenetic protein receptor, type IB/pid=NP_001194.1 Cross-references: MIM:603248; NP_001194.1; AY065994.
(2) E16 (LAT1, SLC7A5, Genbank accession no. NM_003486) Biochem. Biophys. Res. Commun. 255 (2), 283-288 (1999), Nature 395 (6699):288-291 (1998), Gaugitsch, H. W., et al. (1992) J. Biol. Chem. 267 (16):11267-11273); WO2004048938 (Example 2); WO2004032842 (Example IV); WO2003042661 (Claim 12); WO2003016475 (Claim 1); WO200278524 (Example 2); WO200299074 (Claim 19; Page 127-129); WO200286443 (Claim 27; Pages 222, 393); WO2003003906 (Claim 10; Page 293); WO200264798 (Claim 33; Page 93-95); WO200014228 (Claim 5; Page 133-136); US2003224454 (FIG. 3); WO2003025138 (Claim 12; Page 150); NP_003477 solute carrier family 7 (cationic amino acid transporter, y⁺ system), member 5/pid=NP_003477.3—Homo sapiens Cross-references: MIM:600182; NP_003477.3; NM_015923; NM_003486_1.
(3) STEAP1 (six transmembrane epithelial antigen of prostate, Genbank accession no. NM_012449) Cancer Res. 61 (15), 5857-5860 (2001), Hubert, R. S., et al. (1999) Proc. Natl. Acad. Sci. U.S.A. 96 (25):14523-14528); WO2004065577 (Claim 6); WO2004027049 (FIG. 1L); EP1394274 (Example 11); WO2004016225 (Claim 2); WO2003042661 (Claim 12); US2003157089 (Example 5); US2003185830 (Example 5); US2003064397 (FIG. 2); WO200289747 (Example 5; Page 618-619); WO2003022995 (Example 9; FIG. 13A, Example 53; Page 173, Example 2; FIG. 2A); NP_036581 six transmembrane epithelial antigen of the prostate Cross-references: MIM:604415; NP_036581.1; NM_012449_1.
(4) 0772P (CA125, MUC16, Genbank accession no. AF361486) J. Biol. Chem. 276 (29):27371-27375 (2001)); WO2004045553 (Claim 14); WO200292836 (Claim 6; FIG. 12); WO200283866 (Claim 15; Page 116-121); US2003124140 (Example 16); US 798959. Cross-references: GI:34501467; AAK74120.3; AF361486_1.
(5) MPF (MPF, MSLN, SMR, megakaryocyte potentiating factor, mesothelin, Genbank accession no. NM_005823) Yamaguchi, N., et al. Biol. Chem. 269 (2), 805-808 (1994), Proc. Natl. Acad. Sci. U.S.A. 96 (20):11531-11536 (1999), Proc. Natl. Acad. Sci. U.S.A. 93 (1):136-140 (1996), J. Biol. Chem. 270 (37):21984-21990 (1995)); WO2003101283 (Claim 14); (WO2002102235 (Claim 13; Page 287-288); WO2002101075 (Claim 4; Page 308-309); WO200271928 (Page 320-321); WO9410312 (Page 52-57); Cross-references: MIM:601051; NP_005814.2; NM_005823_1.
(6) Napi3b (NAPI-3B, NPTIIb, SLC34A2, solute carrier family 34 (sodium phosphate), member 2, type II sodium-dependent phosphate transporter 3b, Genbank accession no. NM_006424) J. Biol. Chem. 277 (22):19665-19672 (2002), Genomics 62 (2):281-284 (1999), Field, J. A., et al. (1999) Biochem. Biophys. Res. Commun. 258 (3):578-582); WO2004022778 (Claim 2); EP1394274 (Example 11); WO2002102235 (Claim 13; Page 326); EP875569 (Claim 1; Page 17-19); WO200157188 (Claim 20; Page 329); WO2004032842 (Example IV); WO200175177 (Claim 24; Page 139-140); Cross-references: MIM:604217; NP_006415.1; NM_006424_1.
(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (30emaphoring) 5B, Genbank accession no. AB040878) Nagase T., et al. (2000) DNA Res. 7 (2):143-150); WO2004000997 (Claim 1); WO2003003984 (Claim 1); WO200206339 (Claim 1; Page 50); WO200188133 (Claim 1; Page 41-43, 48-58); WO2003054152 (Claim 20); WO2003101400 (Claim 11); Accession: Q9P283; EMBL; AB040878; BAA95969.1. Genew; HGNC:10737.
(8) PSCA hlg (2700050C12Rik, C530008016Rik, RIKEN cDNA 2700050C12, RIKEN cDNA 2700050C12 gene, Genbank accession no. AY358628); Ross et al. (2002) Cancer Res. 62:2546-2553; US2003129192 (Claim 2); US2004044180 (Claim 12); US2004044179 (Claim 11); US2003096961 (Claim 11); US2003232056 (Example 5); WO2003105758 (Claim 12); US2003206918 (Example 5); EP1347046 (Claim 1); WO2003025148 (Claim 20); Cross-references: GI:37182378; AAQ88991.1; AY358628_1.
(9) ETBR (Endothelin type B receptor, Genbank accession no. AY275463); Nakamuta M., et al. Biochem. Biophys. Res. Commun. 177, 34-39, 1991; Ogawa Y., et al. Biochem. Biophys. Res. Commun. 178, 248-255, 1991; Arai H., et al. Jpn. Circ. J. 56, 1303-1307, 1992; Arai H., et al. J. Biol. Chem. 268, 3463-3470, 1993; Sakamoto A., Yanagisawa M., et al. Biochem. Biophys. Res. Commun. 178, 656-663, 1991; Elshourbagy N. A., et al. J. Biol. Chem. 268, 3873-3879, 1993; Haendler B., et al. J. Cardiovasc. Pharmacol. 20, s1-S4, 1992; Tsutsumi M., et al. Gene 228, 43-49, 1999; Strausberg R. L., et al. Proc. Natl. Acad. Sci. U.S.A. 99, 16899-16903, 2002; Bourgeois C., et al. J. Clin. Endocrinol. Metab. 82, 3116-3123, 1997; Okamoto Y., et al. Biol. Chem. 272, 21589-21596, 1997; Verheij J. B., et al. Am. J. Med. Genet. 108, 223-225, 2002; Hofstra R. M. W., et al. Eur. J. Hum. Genet. 5, 180-185, 1997; Puffenberger E. G., et al. Cell 79, 1257-1266, 1994; Attie T., et al., Hum. Mol. Genet. 4, 2407-2409, 1995; Auricchio A., et al. Hum. Mol. Genet. 5:351-354, 1996; Amiel J., et al. Hum. Mol. Genet. 5, 355-357, 1996; Hofstra R. M. W., et al. Nat. Genet. 12, 445-447, 1996; Svensson P. J., et al. Hum. Genet. 103, 145-148, 1998; Fuchs S., et al. Mol. Med. 7, 115-124, 2001; Pingault V., et al. (2002) Hum. Genet. 111, 198-206; WO2004045516 (Claim 1); WO2004048938 (Example 2); WO2004040000 (Claim 151); WO2003087768 (Claim 1); WO2003016475 (Claim 1); WO2003016475 (Claim 1); WO200261087 (FIG. 1); WO2003016494 (FIG. 6); WO2003025138 (Claim 12; Page 144); WO200198351 (Claim 1; Page 124-125); EP522868 (Claim 8; FIG. 2); WO200177172 (Claim 1; Page 297-299); US2003109676; U.S. Pat. No. 6,518,404 (FIG. 3); U.S. Pat. No. 5,773,223 (Claim 1a; Col 31-34); WO2004001004.
(10) MSG783 (RNF124, hypothetical protein FLJ20315, Genbank accession no. NM_017763); WO2003104275 (Claim 1); WO2004046342 (Example 2); WO2003042661 (Claim 12); WO2003083074 (Claim 14; Page 61); WO2003018621 (Claim 1); WO2003024392 (Claim 2; FIG. 93); WO200166689 (Example 6); Cross-references: LocusID:54894; NP_060233.2; NM_017763_1.
(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138) Lab. Invest. 82 (11):1573-1582 (2002)); WO2003087306; US2003064397 (Claim 1; FIG. 1); WO200272596 (Claim 13; Page 54-55); WO200172962 (Claim 1; FIG. 4B); WO2003104270 (Claim 11); WO2003104270 (Claim 16); US2004005598 (Claim 22); WO2003042661 (Claim 12); US2003060612 (Claim 12; FIG. 10); WO200226822 (Claim 23; FIG. 2); WO200216429 (Claim 12; FIG. 10); Cross-references: GI:22655488; AAN04080.1; AF455138_1.
(12) TrpM4 (BR22450, FLJ20041, TRPM4, TRPM4B, transient receptor potential cation channel, subfamily M, member 4, Genbank accession no. NM_017636) Xu, X. Z., et al. Proc. Natl. Acad. Sci. U.S.A. 98 (19):10692-10697 (2001), Cell 109 (3):397-407 (2002), J. Biol. Chem. 278 (33):30813-30820 (2003)); US2003143557 (Claim 4); WO200040614 (Claim 14; Page 100-103); WO200210382 (Claim 1; FIG. 9A); WO2003042661 (Claim 12); WO200230268 (Claim 27; Page 391); US2003219806 (Claim 4); WO200162794 (Claim 14; FIG. 1A-D); Cross-references: MIM:606936; NP_060106.2; NM_017636_1.
(13) CRIPTO (CR, CR1, CRGF, CRIPTO, TDGF1, teratocarcinoma-derived growth factor, Genbank accession no. NP_003203 or NM_003212) Ciccodicola, A., et al. EMBO J. 8 (7):1987-1991 (1989), Am. J. Hum. Genet. 49 (3):555-565 (1991)); US2003224411 (Claim 1); WO2003083041 (Example 1); WO2003034984 (Claim 12); WO200288170 (Claim 2; Page 52-53); WO2003024392 (Claim 2; FIG. 58); WO200216413 (Claim 1; Page 94-95, 105); WO200222808 (Claim 2; FIG. 1); U.S. Pat. No. 5,854,399 (Example 2; Col 17-18); U.S. Pat. No. 5,792,616 (FIG. 2); Cross-references: MIM:187395; NP_003203.1; NM_003212_1.
(14) CD21 (CR2 (Complement receptor 2) or C3DR (C3d/Epstein Barr virus receptor) or Hs.73792 Genbank accession no. M26004) Fujisaku et al. (1989) J. Biol. Chem. 264 (4):2118-2125); Weis J. J., et al. J. Exp. Med. 167, 1047-1066, 1988; Moore M., et al. Proc. Natl. Acad. Sci. U.S.A. 84, 9194-9198, 1987; Barel M., et al. Mol. Immunol. 35, 1025-1031, 1998; Weis J. J., et al. Proc. Natl. Acad. Sci. U.S.A. 83, 5639-5643, 1986; Sinha S. K., et al. (1993) J. Immunol. 150, 5311-5320; WO2004045520 (Example 4); US2004005538 (Example 1); WO2003062401 (Claim 9); WO2004045520 (Example 4); WO9102536 (FIGS. 9.1-9.9); WO2004020595 (Claim 1); Accession: P20023; Q13866; Q14212; EMBL; M26004; AAA35786.1.
(15) CD79b (CD79B, CD79p, Igb (immunoglobulin-associated beta), B29, Genbank accession no. NM_000626 or 11038674) Proc. Natl. Acad. Sci. U.S.A. (2003) 100 (7):4126-4131, Blood (2002) 100 (9):3068-3076, Muller et al. (1992) Eur. J. Immunol. 22 (6):1621-1625); WO2004016225 (claim 2, FIG. 140); WO2003087768, US2004101874 (claim 1, page 102); WO2003062401 (claim 9); WO200278524 (Example 2); US2002150573 (claim 5, page 15); U.S. Pat. No. 5,644,033; WO2003048202 (claim 1, pages 306 and 309); WO 99/558658, U.S. Pat. No. 6,534,482 (claim 13, FIG. 17A/B); WO200055351 (claim 11, pages 1145-1146); Cross-references: MIM:147245; NP 000617.1; NM_000626_1.
(16) FcRH2 (IFGP4, IRTA4, SPAP1A (SH2 domain containing phosphatase anchor protein 1a), SPAP1B, SPAP1C, Genbank accession no. NM_030764, AY358130) Genome Res. 13 (10):2265-2270 (2003), Immunogenetics 54 (2):87-95 (2002), Blood 99 (8):2662-2669 (2002), Proc. Natl. Acad Sci. U.S.A. 98 (17):9772-9777 (2001), Xu, M. J., et al. (2001) Biochem. Biophys. Res. Commun. 280 (3):768-775; WO2004016225 (Claim 2); WO2003077836; WO200138490 (Claim 5; FIG. 18D-1-18D-2); WO2003097803 (Claim 12); WO2003089624 (Claim 25); Cross-references: MIM:606509; NP_110391.2; NM_030764_1.
(17) HER2 (ErbB2, Genbank accession no. M11730) Coussens L., et al. Science (1985) 230(4730):1132-1139); Yamamoto T., et al. Nature 319, 230-234, 1986; Semba K., et al. Proc. Natl. Acad. Sci. U.S.A. 82, 6497-6501, 1985; Swiercz J. M., et al. J. Cell Biol. 165, 869-880, 2004; Kuhns J. J., et al. J. Biol. Chem. 274, 36422-36427, 1999; Cho H.-S., et al. Nature 421, 756-760, 2003; Ehsani A., et al. (1993) Genomics 15, 426-429; WO2004048938 (Example 2); WO2004027049 (FIG. 11); WO2004009622; WO2003081210; WO2003089904 (Claim 9); WO2003016475 (Claim 1); US2003118592; WO2003008537 (Claim 1); WO2003055439 (Claim 29; FIG. 1A-B); WO2003025228 (Claim 37; FIG. 5C); WO200222636 (Example 13; Page 95-107); WO200212341 (Claim 68; FIG. 7); WO200213847 (Page 71-74); WO200214503 (Page 114-117); WO200153463 (Claim 2; Page 41-46); WO200141787 (Page 15); WO200044899 (Claim 52; FIG. 7); WO200020579 (Claim 3; FIG. 2); U.S. Pat. No. 5,869,445 (Claim 3; Col 31-38); WO9630514 (Claim 2; Page 56-61); EP1439393 (Claim 7); WO2004043361 (Claim 7); WO2004022709; WO200100244 (Example 3; FIG. 4); Accession: P04626; EMBL; M11767; AAA35808.1. EMBL; M11761; AAA35808.1.
(18) NCA (CEACAM6, Genbank accession no. M18728); Barnett T., et al. Genomics 3, 59-66, 1988; Iawaragi Y., et al. Biochem. Biophys. Res. Commun. 150, 89-96, 1988; Strausberg R. L., et al. Proc. Natl. Acad. Sci. U.S.A. 99:16899-16903, 2002; WO2004063709; EP1439393 (Claim 7); WO2004044178 (Example 4); WO2004031238; WO2003042661 (Claim 12); WO200278524 (Example 2); WO200286443 (Claim 27; Page 427); WO200260317 (Claim 2); Accession: P40199; Q14920; EMBL; M29541; AAA59915.1. EMBL; M18728.
(19) MDP (DPEP1, Genbank accession no. BC017023) Proc. Natl. Acad. Sci. U.S.A. 99 (26):16899-16903 (2002)); WO2003016475 (Claim 1); WO200264798 (Claim 33; Page 85-87); JP05003790 (FIG. 6-8); WO9946284 (FIG. 9); Cross-references: MIM:179780; AAH17023.1; BC017023_1.
(20) IL20Rα (IL20Ra, ZCYTOR7, Genbank accession no. AF184971); Clark H. F., et al. Genome Res. 13, 2265-2270, 2003; Mungall A. J., et al. Nature 425, 805-811, 2003; Blumberg H., et al. Cell 104, 9-19, 2001; Dumoutier L., et al. J. Immunol. 167, 3545-3549, 2001; Parrish-Novak J., et al. J. Biol. Chem. 277, 47517-47523, 2002; Pletnev S., et al. (2003) Biochemistry 42:12617-12624; Sheikh F., et al. (2004) J. Immunol. 172, 2006-2010; EP1394274 (Example 11); US2004005320 (Example 5); WO2003029262 (Page 74-75); WO2003002717 (Claim 2; Page 63); WO200222153 (Page 45-47); US2002042366 (Page 20-21); WO200146261 (Page 57-59); WO200146232 (Page 63-65); WO9837193 (Claim 1; Page 55-59); Accession: Q9UHF4; Q6UWA9; Q96SH8; EMBL; AF184971; AAF01320.1.
(21) Brevican (BCAN, BEHAB, Genbank accession no. AF229053) Gary S. C., et al. Gene 256, 139-147, 2000; Clark H. F., et al. Genome Res. 13, 2265-2270, 2003; Strausberg R. L., et al. Proc. Natl. Acad. Sci. U.S.A. 99, 16899-16903, 2002; US2003186372 (Claim 11); US2003186373 (Claim 11); US2003119131 (Claim 1; FIG. 52); US2003119122 (Claim 1; FIG. 52); US2003119126 (Claim 1); US2003119121 (Claim 1; FIG. 52); US2003119129 (Claim 1); US2003119130 (Claim 1); US2003119128 (Claim 1; FIG. 52); US2003119125 (Claim 1); WO2003016475 (Claim 1); WO200202634 (Claim 1).
(22) EphB2R (DRT, ERK, Hek5, EPHT3, Tyro5, Genbank accession no. NM_004442) Chan, J. and Watt, V. M., Oncogene 6 (6), 1057-1061 (1991) Oncogene 10 (5):897-905 (1995), Annu. Rev. Neurosci. 21:309-345 (1998), Int. Rev. Cytol. 196:177-244 (2000)); WO2003042661 (Claim 12); WO200053216 (Claim 1; Page 41); WO2004065576 (Claim 1); WO2004020583 (Claim 9); WO2003004529 (Page 128-132); WO200053216 (Claim 1; Page 42); Cross-references: MIM:600997; NP_004433.2; NM_004442_1.
(23) ASLG659 (B7h, Genbank accession no. AX092328) US20040101899 (Claim 2); WO2003104399 (Claim 11); WO2004000221 (FIG. 3); US2003165504 (Claim 1); US2003124140 (Example 2); US2003065143 (FIG. 60); WO2002102235 (Claim 13; Page 299); US2003091580 (Example 2); WO200210187 (Claim 6; FIG. 10); WO200194641 (Claim 12; FIG. 7b); WO200202624 (Claim 13; FIG. 1A-1B); US2002034749 (Claim 54; Page 45-46); WO200206317 (Example 2; Page 320-321, Claim 34; Page 321-322); WO200271928 (Page 468-469); WO200202587 (Example 1; FIG. 1); WO200140269 (Example 3; Pages 190-192); WO200036107 (Example 2; Page 205-207); WO2004053079 (Claim 12); WO2003004989 (Claim 1); WO200271928 (Page 233-234, 452-453); WO 0116318.
(24) PSCA (Prostate stem cell antigen precursor, Genbank accession no. AJ297436) Reiter R. E., et al. Proc. Natl. Acad. Sci. U.S.A. 95, 1735-1740, 1998; Gu Z., et al. Oncogene 19, 1288-1296, 2000; Biochem. Biophys. Res. Commun. (2000) 275(3):783-788; WO2004022709; EP1394274 (Example 11); US2004018553 (Claim 17); WO2003008537 (Claim 1); WO200281646 (Claim 1; Page 164); WO 2003003906 (Claim 10; Page 288); WO 200140309 (Example 1; FIG. 17); US 2001055751 (Example 1; FIG. 1b); WO 200032752 (Claim 18; FIG. 1); WO 1998/51805 (Claim 17; Page 97); WO 1998/51824 (Claim 10; Page 94); WO 1998/40403 (Claim 2; FIG. 1B); Accession: 043653; EMBL; AF043498; AAC39607.1.
(25) GEDA (Genbank accession No. AY260763); AAP14954 lipoma HMGIC fusion-partner-like protein/pid=AAP14954.1—Homo sapiens Species: Homo sapiens (human) WO2003054152 (Claim 20); WO2003000842 (Claim 1); WO2003023013 (Example 3, Claim 20); US2003194704 (Claim 45); Cross-references: GI:30102449; AAP14954.1; AY260763_1.
(26) BAFF-R (B cell-activating factor receptor, BlyS receptor 3, BR3, Genbank accession No. AF116456); BAFF receptor/pid=NP_443177.1—Homo sapiens Thompson, J. S., et al. Science 293 (5537), 2108-2111 (2001); WO2004058309; WO2004011611; WO2003045422 (Example; Page 32-33); WO2003014294 (Claim 35; FIG. 6B); WO2003035846 (Claim 70; Page 615-616); WO200294852 (Col 136-137); WO200238766 (Claim 3; Page 133); WO200224909 (Example 3; FIG. 3); Cross-references: MIM:606269; NP_443177.1; NM_052945_1; AF132600.
(27) CD22 (B-cell receptor CD22-B isoform, BL-CAM, Lyb-8, Lyb8, SIGLEC-2, FLJ22814, Genbank accession No. AK026467); Wilson et al. (1991) J. Exp. Med. 173:137-146; WO2003072036 (Claim 1; FIG. 1); Cross-references: MIM:107266; NP_001762.1; NM_001771_1.
(28) CD79a (CD79A, CD79c, immunoglobulin-associated alpha, a B cell-specific protein that covalently interacts with Ig beta (CD79B) and forms a complex on the surface with Ig M molecules, transduces a signal involved in B-cell differentiation), pI: 4.84, MW: 25028 TM: 2 [P] Gene Chromosome: 19q13.2, Genbank accession No. NP_001774.10) WO2003088808, US20030228319; WO2003062401 (claim 9); US2002150573 (claim 4, pages 13-14); WO9958658 (claim 13, FIG. 16); WO9207574 (FIG. 1); U.S. Pat. No. 5,644,033; Ha et al. (1992) J. Immunol. 148(5):1526-1531; Mueller et al. (1992) Eur. J. Biochem. 22:1621-1625; Hashimoto et al. (1994) Immunogenetics 40(4):287-295; Preud'homme et al. (1992) Clin. Exp. Immunol. 90(1):141-146; Yu et al. (1992) J. Immunol. 148(2) 633-637; Sakaguchi et al. (1988) EMBO J. 7(11):3457-3464.
(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, pI: 8.54 MW: 41959 TM: 7 [P] Gene Chromosome: 11q23.3, Genbank accession No. NP_001707.1) WO 2004040000; WO2004/015426; US2003105292 (Example 2); U.S. Pat. No. 6,555,339 (Example 2); WO 2002/61087 (FIG. 1); WO200157188 (Claim 20, page 269); WO200172830 (pages 12-13); WO 2000/22129 (Example 1, pages 152-153, Example 2, pages 254-256); WO 199928468 (claim 1, page 38); U.S. Pat. No. 5,440,021 (Example 2, col 49-52); WO9428931 (pages 56-58); WO 1992/17497 (claim 7, FIG. 5); Dobner et al. (1992) Eur. J. Immunol. 22:2795-2799; Barella et al. (1995) Biochem. J. 309:773-779.
(30) HLA-DOB (Beta subunit of MHC class II molecule (Ia antigen) that binds peptides and presents them to CD4+T lymphocytes); 273 aa, pI: 6.56 MW: 30820 TM: 1 [P] Gene Chromosome: 6p21.3, Genbank accession No. NP_002111.1) Tonnelle et al. (1985) EMBO J. 4(11):2839-2847; Jonsson et al. (1989) Immunogenetics 29(6):411-413; Beck et al. (1992) J. Mol. Biol. 228:433-441; Strausberg et al. (2002) Proc. Natl. Acad Sci USA 99:16899-16903; Servenius et al. (1987) J. Biol. Chem. 262:8759-8766; Beck et al. (1996) J. Mot. Biol. 255:1-13; Naruse et al. (2002) Tissue Antigens 59:512-519; WO9958658 (claim 13, FIG. 15); U.S. Pat. No. 6,153,408 (Col 35-38); U.S. Pat. No. 5,976,551 (col 168-170); U.S. Pat. No. 6,011,146 (col 145-146); Kasahara et al. (1989) Immunogenetics 30(1):66-68; Larhammar et al. (1985) J. Biol. Chem. 260(26):14111-14119.
(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pI: 7.63, MW: 47206 TM: 1 [P] Gene Chromosome: 17p13.3, Genbank accession No. NP_002552.2) Le et al. (1997) FEBS Lett. 418(1-2):195-199; WO2004047749; WO2003072035 (claim 10); Touchman et al. (2000) Genome Res. 10:165-173; WO200222660 (claim 20); WO2003093444 (claim 1); WO2003087768 (claim 1); WO2003029277 (page 82).
(32) CD72 (B-cell differentiation antigen CD72, Lyb-2), pI: 8.66, MW: 40225 TM: 1 [P] Gene Chromosome: 9p13.3, Genbank accession No. NP_001773.1) WO2004042346 (claim 65); WO 2003/026493 (pages 51-52, 57-58); WO 2000/75655 (pages 105-106); Von Hoegen et al. (1990) J. Immunol. 144(12):4870-4877; Strausberg et al. (2002) Proc. Natl. Acad. Sci USA 99:16899-16903.
(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosus); 661 aa, pI: 6.20, MW: 74147 TM: 1 [P] Gene Chromosome: Sq12, Genbank accession No. NP_005573.1) US2002193567; WO9707198 (claim 11, pages 39-42); Miura et al. (1996) Genomics 38(3):299-304; Miura et al. (1998) Blood 92:2815-2822; WO2003083047; WO9744452 (claim 8, pages 57-61); WO200012130 (pages 24-26).
(34) FcRH1 (Fc receptor-like protein 1, a putative receptor for the immunoglobulin Fc domain that contains C2 type Ig-like and ITAM domains, may have a role in B-lymphocyte differentiation); 429 aa, pI: 5.28, MW: 46925 TM: 1 [P] Gene Chromosome: 1q21-1q22, Genbank accession No. NP_443170.1) WO2003077836; WO200138490 (claim 6, FIG. 18E-1-18-E-2); Davis et al. (2001) Proc. Natl. Acad. Sci USA 98(17):9772-9777; WO2003089624 (claim 8); EP1347046 (claim 1); WO2003089624 (claim 7).
(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pI: 6.88 MW: 106468 TM: 1 [P] Gene Chromosome: 1q21, Genbank accession No. Human: AF343662, AF343663, AF343664, AF343665, AF369794, AF397453, AK090423, AK090475, AL834187, AY358085; Mouse: AK089756, AY158090, AY506558; NP_112571.1. WO2003024392 (claim 2, FIG. 97); Nakayama et al. (2000) Biochem. Biophys. Res. Commun. 277(1):124-127; WO2003077836; WO200138490 (claim 3, FIG. 18B-1-18B-2).
(36) TENB2 (TMEFF2, tomoregulin, TPEF, HPP1, TR, putative transmembrane proteoglycan, related to the EGF/heregulin family of growth factors and follistatin); 374 aa, NCBI Accession: AAD55776, AAF91397, AAG49451, NCBI RefSeq: NP_057276; NCBI Gene: 23671; OMIM: 605734; SwissProt Q9UIK5; Genbank accession No. AF179274; AY358907, CAF85723, CQ782436 WO 2004074320; JP 2004113151; WO 2003042661; WO2003009814; EP1295944 (pages 69-70); WO 200230268 (page 329); WO 200190304; US2004249130; US 2004022727; WO 2004063355; US 2004197325; US2003232350; US2004005563; US 2003124579; Horie et al. (2000) Genomics 67:146-152; Uchida et al. (1999) Biochem. Biophys. Res. Commun. 266:593-602; Liang et al. (2000) Cancer Res. 60:4907-12; Glynne-Jones et al. (2001) Int J Cancer. October 15; 94(2):178-84.
(37) PMEL17 (silver homolog; SILV; D12S53E; PMEL17; SI; SIL); ME20; gp100) BC001414; BT007202; M32295; M77348; NM_006928; McGlinchey, R. P. et al. (2009) Proc. Natl. Acad Sci. U.S.A. 106 (33), 13731-13736; Kummer, M. P. et al. (2009) J. Biol. Chem. 284 (4), 2296-2306.
(38) TMEFF1 (transmembrane protein with EGF-like and two follistatin-like domains 1; Tomoregulin-1); H7365; C9orf2; C90RF2; U19878; X83961; NM_080655; NM_003692; Harms, P. W. (2003) Genes Dev. 17 (21), 2624-2629; Gery, S. et al. (2003) Oncogene 22 (18):2723-2727.
(39) GDNF-Ra1 (GDNF family receptor alpha 1; GFRA1; GDNFR; GDNFRA; RETL1; TRNR1; RET1L; GDNFR-alpha1; GFR-ALPHA-1); U95847; BC014962; NM_145793 NM_005264; Kim, M. H. et al. (2009) Mol. Cell. Biol. 29 (8), 2264-2277; Treanor, J. J. et al. (1996) Nature 382 (6586):80-83.
(40) Ly6E (lymphocyte antigen 6 complex, locus E; Ly67, RIG-E, SCA-2, TSA-1); NP_002337.1; NM_002346.2; de Nooij-van Dalen, A. G. et al. (2003) Int. J. Cancer 103 (6), 768-774; Zammit, D. J. et al (2002) Mol. Cell. Biol. 22 (3):946-952.
(41) TMEM46 (shisa homolog 2 (Xenopus laevis); SHISA2); NP_001007539.1; NM_001007538.1; Furushima, K. et al. (2007) Dev. Biol. 306 (2), 480-492; Clark, H. F. et al. (2003) Genome Res. 13 (10):2265-2270.
(42) Ly6G6D (lymphocyte antigen 6 complex, locus G6D; Ly6-D, MEGT1); NP 067079.2; NM_021246.2; Mallya, M. et al. (2002) Genomics 80 (1):113-123; Ribas, G. et al. (1999) J. Immunol. 163 (1):278-287.
(43) LGR5 (leucine-rich repeat-containing G protein-coupled receptor 5; GPR49, GPR67); NP_003658.1; NM_003667.2; Salanti, G. et al. (2009) Am. J. Epidemiol. 170 (5):537-545; Yamamoto, Y. et al. (2003) Hepatology 37 (3):528-533.
(44) RET (ret proto-oncogene; MEN2A; HSCR1; MEN2B; MTC1; PTC; CDHF12; Hs.168114; RET51; RET-ELE1); NP_066124.1; NM_020975.4; Tsukamoto, H. et al. (2009) Cancer Sci. 100 (10):1895-1901; Narita, N. et al. (2009) Oncogene 28 (34):3058-3068.
(45) LY6K (lymphocyte antigen 6 complex, locus K; LY6K; HSJ001348; FLJ35226); NP_059997.3; NM_017527.3; Ishikawa, N. et al. (2007) Cancer Res. 67 (24):11601-11611; de Nooij-van Dalen, A. G. et al. (2003) Int. J. Cancer 103 (6):768-774.
(46) GPR19 (G protein-coupled receptor 19; Mm.4787); NP_006134.1; NM_006143.2; Montpetit, A. and Sinnett, D. (1999) Hum. Genet. 105 (1-2):162-164; O'Dowd, B. F. et al. (1996) FEBS Lett. 394 (3):325-329.
(47) GPR54 (KISS1 receptor; KISS1R; GPR54; HOT7T175; AXOR12); NP_115940.2; NM_032551.4; Navenot, J. M. et al. (2009) Mol. Pharmacol. 75 (6):1300-1306; Hata, K. et al. (2009) Anticancer Res. 29 (2):617-623.
(48) ASPHD1 (aspartate beta-hydroxylase domain containing 1; LOC253982); NP_859069.2; NM_181718.3; Gerhard, D. S. et al. (2004) Genome Res. 14 (10B):2121-2127.
(49) Tyrosinase (TYR; OCAIA; OCAIA; tyrosinase; SHEP3); NP_000363.1; NM_000372.4; Bishop, D. T. et al. (2009) Nat. Genet. 41 (8):920-925; Nan, H. et al. (2009) Int. J. Cancer 125 (4):909-917.
(50) TMEM118 (ring finger protein, transmembrane 2; RNFT2; FLJ14627); NP_001103373.1; NM_001109903.1; Clark, H. F. et al. (2003) Genome Res. 13 (10):2265-2270; Scherer, S. E. et al. (2006) Nature 440 (7082):346-351.
(51) GPR172A (G protein-coupled receptor 172A; GPCR41; FLJ11856; D15Ertd747e); NP_078807.1; NM_024531.3; Ericsson, T. A. et al. (2003) Proc. Natl. Acad. Sci. U.S.A. 100 (11):6759-6764; Takeda, S. et al. (2002) FEBS Lett. 520 (1-3):97-101.
(52) CD33, a member of the sialic acid binding, immunoglobulin-like lectin family, is a 67-kDa glycosylated transmembrane protein. CD33 is expressed on most myeloid and monocytic leukemia cells in addition to committed myelomonocytic and erythroid progenitor cells. It is not seen on the earliest pluripotent stem cells, mature granulocytes, lymphoid cells, or nonhematopoietic cells (Sabbath et al., (1985) J. Clin. Invest. 75:756-56; Andrews et al., (1986) Blood 68:1030-5). CD33 contains two tyrosine residues on its cytoplasmic tail, each of which is followed by hydrophobic residues similar to the immunoreceptor tyrosine-based inhibitory motif (ITIM) seen in many inhibitory receptors.
(53) CLL-1 (CLEC12A, MICL, and DCAL2), encodes a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. The protein encoded by this gene is a negative regulator of granulocyte and monocyte function. Several alternatively spliced transcript variants of this gene have been described, but the full-length nature of some of these variants has not been determined. This gene is closely linked to other CTL/CTLD superfamily members in the natural killer gene complex region on chromosome 12p13 (Drickamer K (1999) Curr. Opin. Struct. Biol. 9 (5):585-90; van Rhenen A, et al., (2007) Blood 110 (7):2659-66; Chen C H, et al. (2006) Blood 107 (4):1459-67; Marshall A S, et al. (2006) Eur. J. Immunol. 36 (8):2159-69; Bakker A B, et al. (2005) Cancer Res. 64 (22):8443-50; Marshall A S, et al. (2004) J. Biol. Chem. 279 (15):14792-802). CLL-1 has been shown to be a type II transmembrane receptor comprising a single C-type lectin-like domain (which is not predicted to bind either calcium or sugar), a stalk region, a transmembrane domain and a short cytoplasmic tail containing an ITIM motif.
(54) TROP2 (tumor-associated calcium signal transducer 2) is a transmembrane glycoprotein encoded by the TACSTD2 gene (Linnenbach A J, et al (1993) Mol Cell Biol. 13(3): 1507-15; Calabrese G, et al (2001) Cytogenet Cell Genet. 92(1-2): 164-5). TROP2 is an intracellular calcium signal transducer that is differentially expressed in many cancers. It signals cells for self-renewal, proliferation, invasion, and survival. It has stem cell-like qualities. TROP2 is expressed in many normal tissues, though in contrast, it is overexpressed in many cancers (Ohmachi T, et al., (2006) Clin. Cancer Res., 12(10):3057-3063; Muhlmann G, et al., (2009) J. Clin. Pathol., 62(2):152-158; Fong D, et al., (2008) Br. J. Cancer, 99(8):1290-1295; Fong D, et al., (2008) Mod. Pathol., 21(2):186-191; Ning S, et al., (2013) Neurol. Sci., 34(10):1745-1750). Overexpression of TROP2 is of prognostic significance. Several ligands have been proposed that interact with TROP2. TROP2 signals the cells via different pathways and it is transcriptionally regulated by a complex network of several transcription factors.

Human TROP2 (TACSTD2: tumor-associated calcium signal transducer 2, GA733-1, EGP-1, M1S1; hereinafter, referred to as hTROP2) is a single-pass transmembrane type 1 cell membrane protein consisting of 323 amino acid residues. While the presence of a cell membrane protein involved in immune resistance, which is common to human trophoblasts and cancer cells (Faulk W P, et al. (1978), Proc. Natl. Acad. Sci. 75(4):1947-1951), has previously been suggested, an antigen molecule recognized by a monoclonal antibody against a cell membrane protein in a human choriocarcinoma cell line was identified and designated as TROP2 as one of the molecules expressed In human trophoblasts (Lipinski M, et al. (1981), Proc. Natl. Acad. Sci. 78(8), 5147-5150). This molecule was also designated as tumor antigen GA733-1 recognized by a mouse monoclonal antibody GA733 (Linnenbach A J, et al., (1989) Proc. Natl. Acad. Sci. 86(1), 27-31) obtained by immunization with a gastric cancer cell line or an epithelial glycoprotein (EGP-1; Basu A, et al., Int. J. Cancer, 62 (4), 472-479 (1995)) recognized by a mouse monoclonal antibody RS7-3G11 obtained by immunization with non-small cell lung cancer cells. In 1995, however, the TROP2 gene was cloned, and all of these molecules were confirmed to be identical molecules (Fornaro M, et al., (1995) Int. J. Cancer, 62(5), 610-618). The DNA sequence and amino acid sequence of hTROP2 are available on a public database and can be referred to, for example, under Accession Nos. NM_002353 and NP_002344 (NCBI).

### Isoindolinone-Glutarimide Compounds

The antibody conjugate composition of the invention comprises an isoindolinone-glutarimide moiety (IG). The antibody conjugate composition of the present disclosure may be prepared from an isoindolinone-glutarimide (IG) compound selected from Formula III:

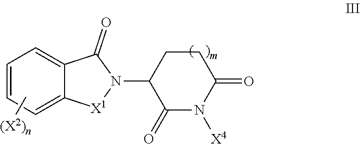

or a pharmaceutically acceptable salt, stereoisomer, mixture of stereoisomers, tautomer, or isotopic analog thereof, wherein:
m is 0, 1 or 2;
X¹ is selected from the group consisting of CH₂, C(═O) and N═N;
X² is independently selected from the group consisting of F, Cl, Br, I, —CN, C₁-C₁₂ alkyl, C₂-C₁₂ alkenyl, C₂-C₁₂ alkynyl, C₁-C₁₂ heteroalkyl, —(C₁-C₁₂ heteroalkyldiyl)-(C₆-C₂₀ aryldiyl), —(C₁-C₁₂ heteroalkyldiyl)-(C₆-C₂₀ aryldiyl)-(C₁-C₁₂ heteroalkyl), (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C3-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)R^{a}—NR^{a}R^{b}, —C(═O)NH(C₁-C₆ alkyldiyl)-NR^{a}R^{b}, —C(═O)OR^{a}, —NR^{a}R^{b}, —NO₂, —OR^{a}, —OC(═O)R^{a}, —SR^{a}, —S(O)R^{a}, —S(O)₂R^{a}, —S(O)₂NR^{a}, and —S(O)₃H;
R^{a} is independently selected from H, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
R^{b} is independently selected from H, OH, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
n is 0, 1, 2, 3, or 4; and
X⁴ is selected from the group consisting of H, C₁-C₁₂ heteroalkyl, —(C₁-C₁₂ heteroalkyldiyl)-(C₆-C₂₀ aryl), —(C₁-C₁₂ heteroalkyldiyl)-(C₆-C₂₀ aryldiyl)-(C₁-C₁₂ heteroalkyl);
wherein each alkyl, alkyldiyl, alkenyl, alkenyldiyl, alkynyl, alkynyldiyl, heteroalkyl, heteroalkyldiyl, aryl, aryldiyl, carbocyclyl, carbocyclyldiyl, heterocyclyl, heterocyclyldiyl, heteroaryl, and heteroaryldiyl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of the isoindolinone-glutarimide compound includes wherein m is 0.

An exemplary embodiment of the isoindolinone-glutarimide compound includes wherein m is 1.

An exemplary embodiment of the isoindolinone-glutarimide compound includes wherein m is 2.

An exemplary embodiment of the isoindolinone-glutarimide compound includes wherein X¹ is CH₂.

An exemplary embodiment of the isoindolinone-glutarimide compound includes wherein X¹ is C(═O).

An exemplary embodiment of the isoindolinone-glutarimide compound includes wherein X¹ is N═N.

An exemplary embodiment of the isoindolinone-glutarimide compound includes wherein X² comprises a —NHC(═O)NH— group.

An exemplary embodiment of the isoindolinone-glutarimide compound includes wherein X² is selected from the group consisting of C₁-C₁₂ heteroalkyl, —(C₁-C₁₂ heteroalkyldiyl)-(C₆-C₂₀ aryl), and —(C₁-C₁₂ heteroalkyldiyl)-(C₆-C₂₀ aryldiyl)-(C₁-C₁₂ heteroalkyl).

An exemplary embodiment of the isoindolinone-glutarimide compound includes wherein X² is —(C₁-C₁₂ heteroalkyldiyl)-(C₆-C₂₀ aryl).

An exemplary embodiment of the isoindolinone-glutarimide compound includes wherein X² is —CH₂NHC(═O)NH—(C₆-C₂₀ aryl).

An exemplary embodiment of the isoindolinone-glutarimide compound includes wherein X² is:

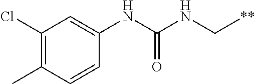

where ** indicates the point of attachment.

An exemplary embodiment of the isoindolinone-glutarimide compound includes wherein X⁴ is H.

An exemplary embodiment of the isoindolinone-glutarimide compound includes wherein X⁴ is selected from the group consisting of C₁-C₁₂ heteroalkyl, —(C₁-C₁₂ heteroalkyldiyl)-(C₆-C₂₀ aryl), —(C₁-C₁₂ heteroalkyldiyl)-(C₆-C₂₀ aryldiyl)-(C₁-C₁₂ heteroalkyl), and —(C₁-C₁₂ heteroalkyldiyl)-(C₆-C₂₀ aryldiyl)-O—(C₂-C₂₀ heterocyclyl).

An exemplary embodiment of the isoindolinone-glutarimide compound includes wherein X⁴ is —(C₁-C₁₂ heteroalkyldiyl)-(C₆-C₂₀ aryldiyl)-O—(C₂-C₂₀ heterocyclyl), and C₂-C₂₀ heterocyclyl is a glucuronide.

An exemplary embodiment of the isoindolinone-glutarimide compound includes wherein X⁴ of formula Ia is selected from the formulae:

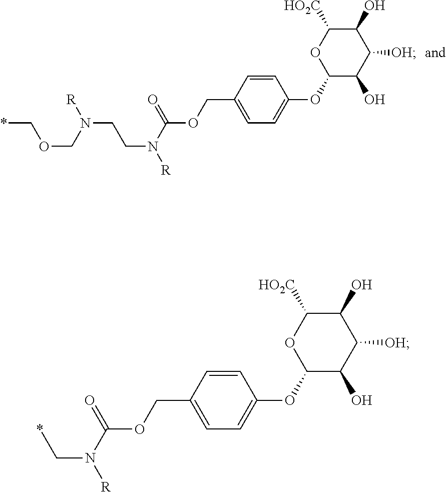

and
wherein R is selected from H, C₁-C₆ alkyl, and O—(C₁-C₆ alkyl); and * indicates the point of attachment.

Exemplary isoindolinone-glutarimide (IG) compounds of Table 1a, 1b and 1c were prepared and characterized according to the Examples herein. Certain exemplary IG compounds of(Table 1a were tested for their effects in inhibiting cellular proliferation, including CAL1 WSU-DLCL2, NCI-N87 and SKBR3. CAL51 is a human breast adenocarcinoma cell line with triple-negative status for expression of estrogen, progesterone and HER2 receptors. WSU-DLCL2 is a human B-Cell non-Hodgkin lymphoma cell line that expresses high levels of CD22. NCI-N87 is a human epithelial cell line established from a gastric carcinoma; SKBR3 is a human epithelial cell line established from a breast adenocarcinoma; both NCI-N87 and SKBR3 cell lines express high levels of HER2 receptor.

TABLE la
Isoindolinone-glutarimide (IG) compounds

| IG No. | Structure | Name | MW |
|---|---|---|---|
| IG-1 | 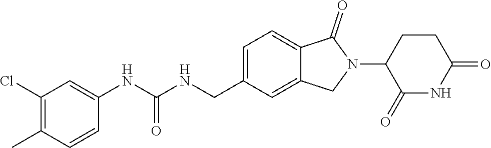 [SMILES: Cc1ccc(NC(=O)NCc2ccc3c(c2)CN(C2CCC(=O)NC2=O)C3=O)cc1Cl] | 1-(3-chloro-4-methylphenyl)-3-((2- (2,6-dioxopiperidin-3-yl)-1- oxoisoindolin-5-yl)methyl)urea | 440.9 |
| IG-2 | 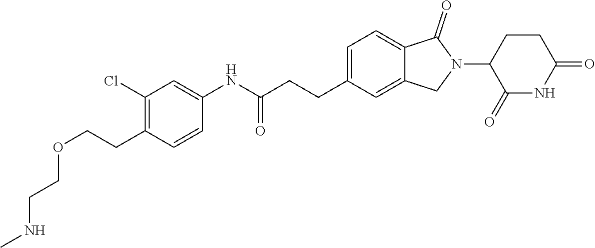 [SMILES: CNCCOCCc1ccc(NC(=O)CCc2ccc3c(c2)CN(C2CCC(=O)NC2=O)C3=O)cc1Cl] | N-(3-chloro-4-(2-(2- (methylamino)ethoxy)ethyl)phenyl)- 3-(2-(2,6-dioxopiperidin-3-yl)-1- oxoisoindolin-5-yl)propanamide | 527.0 |
| IG-3 | 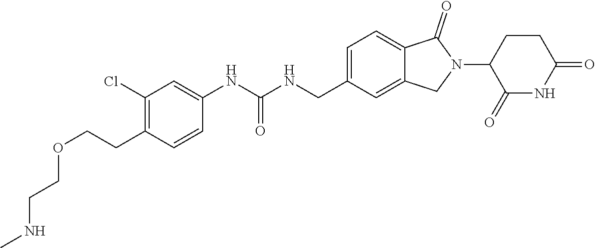 [SMILES: CNCCOCCc1ccc(NC(=O)NCc2ccc3c(c2)CN(C2CCC(=O)NC2=O)C3=O)cc1Cl] | 1-(3-chloro-4-(2-(2- (methylamino)ethoxy)ethyl)phenyl)- 3-((2-(2,6-dioxopiperidin-3-yl)-1- oxoisoindolin-5-yl)methyl)urea | 528.0 |
| IG-4 | 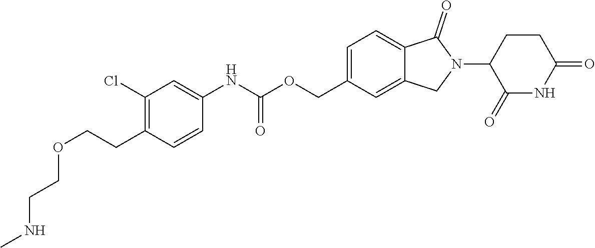 [SMILES: CNCCOCCc1ccc(NC(=O)OCc2ccc3c(c2)CN(C2CCC(=O)NC2=O)C3=O)cc1Cl] | (2-(2,6-dioxopiperidin-3-yl)-1- oxoisoindolin-5-yl)methyl (3-chloro- 4-(2-(2- (methylamino)ethoxy)ethyl)phenyl) carbamate | 529.0 |
| IG-5 | 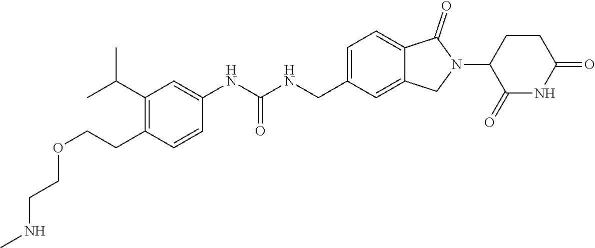 [SMILES: CNCCOCCc1ccc(NC(=O)NCc2ccc3c(c2)CN(C2CCC(=O)NC2=O)C3=O)cc1C(C)C] | 1-((2-(2,6-dioxopiperidin-3-yl)-1- oxoisoindolin-5-yl)methyl)-3-(3- isopropyl-4-(2-(2- (methylamino)ethoxy)ethyl)phenyl) urea | 535.6 |
| IG-6 | 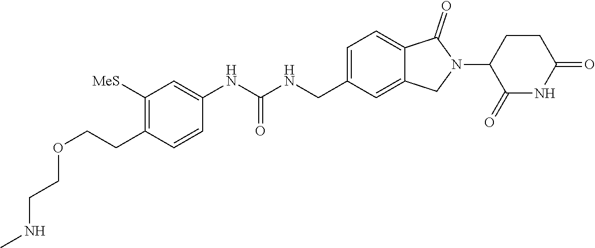 [SMILES: CNCCOCCc1ccc(NC(=O)NCc2ccc3c(c2)CN(C2CCC(=O)NC2=O)C3=O)cc1SC] | 1-((2-(2,6-dioxopiperidin-3-yl)-1- oxoisoindolin-5-yl)methyl)-3-(4-(2- (2-(methylamino)ethoxy)ethyl)-3- (methylthio)phenyl)urea | 539.7 |
| IG-7 | 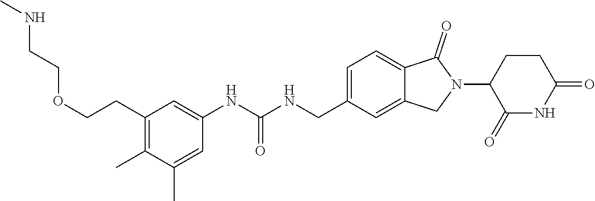 [SMILES: CNCCOCCc1cc(NC(=O)NCc2ccc3c(c2)CN(C2CCC(=O)NC2=O)C3=O)cc(C)c1C] | 1-((2-(2,6-dioxopiperidin-3-yl)-1- oxoisoindolin-5-yl)methyl)-3-(3-(2- ((2-methoxyethyl)amino)ethyl)-4,5- dimethylphenyl)urea | 521.6 |
| IG-8 | 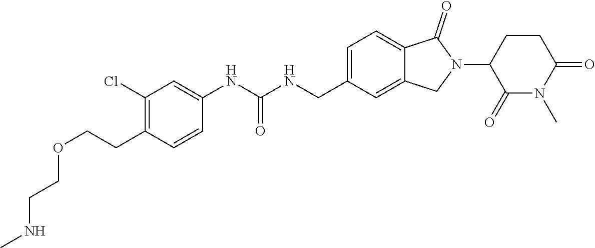 [SMILES: CNCCOCCc1ccc(NC(=O)NCc2ccc3c(c2)CN(C2CCC(=O)N(C)C2=O)C3=O)cc1Cl] | 1-(3-chloro-4-(2-(2- (methylamino)ethoxy)ethyl)phenyl)- 3-((2-(1-methyl-2,6-dioxopiperidin- 3-yl)-1-oxoisoindolin-5- yl)methyl)urea | 542.0 |
| IG-9 | 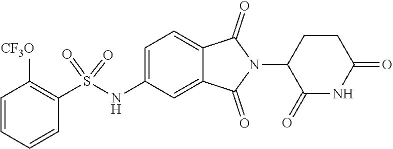 | N-(2-(2,6-dioxopiperidin-3-yl)-1,3- dioxoisoindolin-5-yl)-2- (trifluoromethoxy) benzenesulfonamide | 497.4 |
| IG-10 | 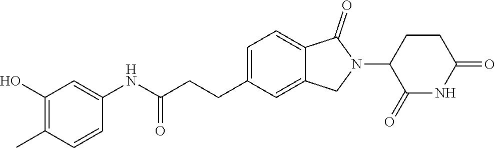 [SMILES: Cc1ccc(NC(=O)CCc2ccc3c(c2)CN(C2CCC(=O)NC2=O)C3=O)cc1O] | 3-(2-(2,6-dioxopiperidin-3-yl)-1- oxoisoindolin-5-yl)-N-(3-hydroxy-4- methylphenyl)propanamide | 421.5 |
| IG-11 | 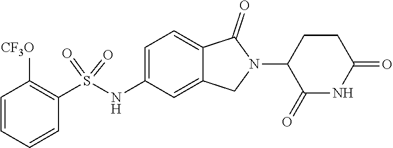 | N-(2-(2,6-dioxopiperidin-3-yl)-1- oxoisoindolin-5-yl)-2- (trifluoromethoxy) benzenesulfonamide | 483.4 |
| IG-12 | 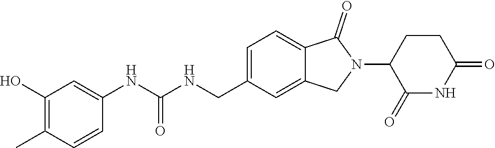 [SMILES: Cc1ccc(NC(=O)NCc2ccc3c(c2)CN(C2CCC(=O)NC2=O)C3=O)cc1O] | 1-((2-(2,6-dioxopiperidin-3-yl)-1- oxoisoindolin-5-yl)methyl)-3-(3- hydroxy-4-methylphenyl)urea | 422.4 |
| IG-13 | 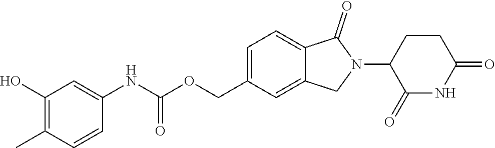 [SMILES: Cc1ccc(NC(=O)OCc2ccc3c(c2)CN(C2CCC(=O)NC2=O)C3=O)cc1O] | (2-(2,6-dioxopiperidin-3-yl)-1- oxoisoindolin-5-yl)methyl (3- hydroxy-4-methylphenyl)carbamate | 423.4 |
| IG-14 | 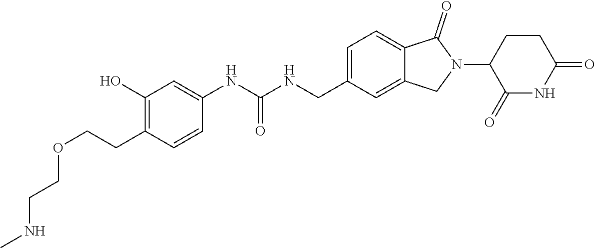 [SMILES: CNCCOCCc1ccc(NC(=O)NCc2ccc3c(c2)CN(C2CCC(=O)NC2=O)C3=O)cc1O] | 1-((2-(2,6-dioxopiperidin-3-yl)-1- oxoisoindolin-5-yl)methyl)-3-(3- hydroxy-4-(2-(2- (methylamino)ethoxy)ethyl)phenyl) urea | 509.6 |
| IG-15 | 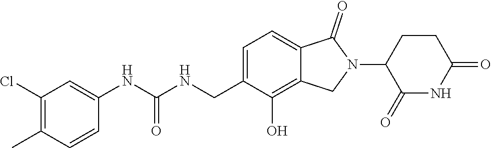 [SMILES: Cc1ccc(NC(=O)NCc2ccc3c(c2O)CN(C2CCC(=O)NC2=O)C3=O)cc1Cl] | 1-(3-chloro-4-methylphenyl)-3-((2- (2,6-dioxopiperidin-3-yl)-4-hydroxy- 1-oxoisoindolin-5-yl)methyl)urea | 456.9 |
| IG-16 | 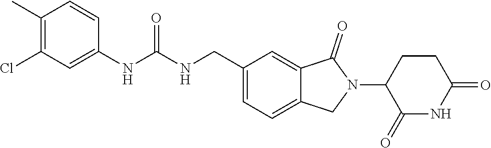 [SMILES: Cc1ccc(NC(=O)NCc2ccc3c(c2)C(=O)N(C2CCC(=O)NC2=O)C3)cc1Cl] | 1-(3-chloro-4-methylphenyl)-3-((2- (2,6-dioxopiperidin-3-yl)-3- oxoisoindolin-5-yl)methyl)urea | 440.9 |
| IG-17 | 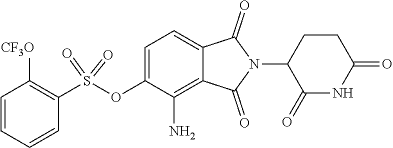 | 4-amino-2-(2,6-dioxopiperidin-3-yl)- 1,3-dioxoisoindolin-5-yl 2- (trifluoromethoxy)benzenesulfonate | 513.4 |
| IG-18 | 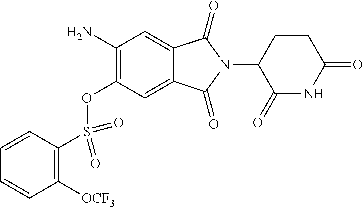 [SMILES: Nc1cc2c(cc1OS(=O)(=O)c1ccccc1OC(F)(F)F)C(=O)N(C1CCC(=O)NC1=O)C2=O] | 6-amino-2-(2,6-dioxopiperidin-3-yl)- 1,3-dioxoisoindolin-5-yl 2- (trifluoromethoxy)benzenesulfonate | 513.4 |
| IG-19 | 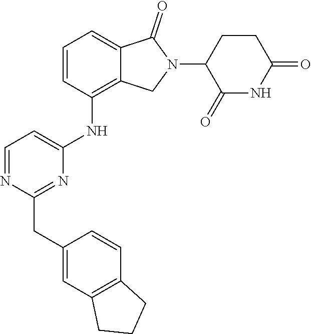 [SMILES: O=C1CCC(N2Cc3c(Nc4ccnc(Cc5ccc6c(c5)CCC6)n4)cccc3C2=O)C(=O)N1] | 3-(4-((2-((2,3-dihydro-1H-inden-5- yl)methyl)pyrimidin-4-yl)amino)-1- oxoisoindolin-2-yl)piperidine-2,6- dione | 467.5 |
| IG-20 | 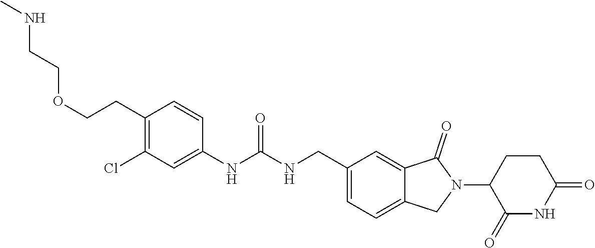 [SMILES: CNCCOCCc1ccc(NC(=O)NCc2ccc3c(c2)C(=O)N(C2CCC(=O)NC2=O)C3)cc1Cl] | 1-(3-chloro-4-(2-(2- (methylamino)ethoxy)ethyl)phenyl)- 3-((2-(2,6-dioxopiperidin-3-yl)-3- oxoisoindolin-5-yl)methyl)urea | 528.0 |
| IG-21 | 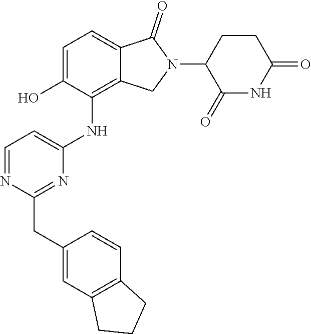 [SMILES: O=C1CCC(N2Cc3c(ccc(O)c3Nc3ccnc(Cc4ccc5c(c4)CCC5)n3)C2=O)C(=O)N1] | 3-(4-((2-((2,3-dihydro-1H-inden-5- yl)methyl)pyrimidin-4-yl)amino)-5- hydroxy-1-oxoisoindolin-2- yl)piperidine-2,6-dione | 483.5 |
| IG-22 | 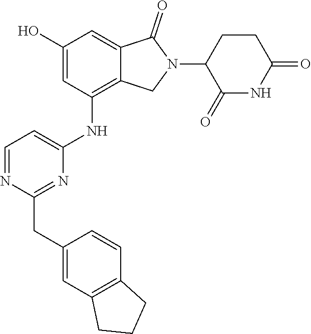 [SMILES: O=C1CCC(N2Cc3c(Nc4ccnc(Cc5ccc6c(c5)CCC6)n4)cc(O)cc3C2=O)C(=O)N1] | 3-(4-((2-((2,3-dihydro-1H-inden-5- yl)methyl)pyrimidin-4-yl)amino)-6- hydroxy-1-oxoisoindolin-2- yl)piperidine-2,6-dione | 483.5 |
| IG-23 | 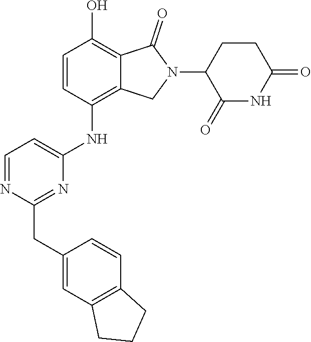 [SMILES: O=C1CCC(N2Cc3c(Nc4ccnc(Cc5ccc6c(c5)CCC6)n4)ccc(O)c3C2=O)C(=O)N1] | 3-(4-((2-((2,3-dihydro-1H-inden-5- yl)methyl)pyrimidin-4-yl)amino)-7- hydroxy-1-oxoisoindolin-2- yl)piperidine-2,6-dione | 483.5 |
| IG-24 | 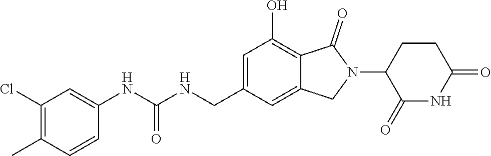 [SMILES: Cc1ccc(NC(=O)NCc2cc(O)c3c(c2)CN(C2CCC(=O)NC2=O)C3=O)cc1Cl] | 1-(3-chloro-4-methylphenyl)-3-((2- (2,6-dioxopiperidin-3-yl)-7-hydroxy- 1-oxoisoindolin-5-yl)methyl)urea | 456.9 |
| IG-25 | 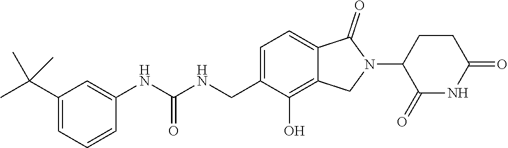 [SMILES: CC(C)(C)c1cccc(NC(=O)NCc2ccc3c(c2O)CN(C2CCC(=O)NC2=O)C3=O)c1] | 1-(3-(tert-butyl)phenyl)-3-((2-(2,6- dioxopiperidin-3-yl)-4-hydroxy-1- oxoisoindolin-5-yl)methyl)urea | 464.5 |
| IG-26 | 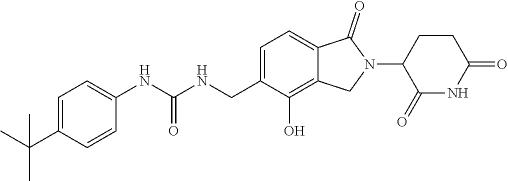 [SMILES: CC(C)(C)c1ccc(NC(=O)NCc2ccc3c(c2O)CN(C2CCC(=O)NC2=O)C3=O)cc1] | 1-(4-(tert-butyl)phenyl)-3-((2-(2,6- dioxopiperidin-3-yl)-4-hydroxy-1- oxoisoindolin-5-yl)methyl)urea | 464.5 |
| IG-27 | 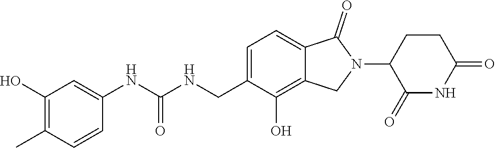 [SMILES: Cc1ccc(NC(=O)NCc2ccc3c(c2O)CN(C2CCC(=O)NC2=O)C3=O)cc1O] | 1-((2-(2,6-dioxopiperidin-3-yl)-4- hydroxy-1-oxoisoindolin-5- yl)methyl)-3-(3-hydroxy-4- methylphenyl)urea | 438.4 |
| IG-28 | 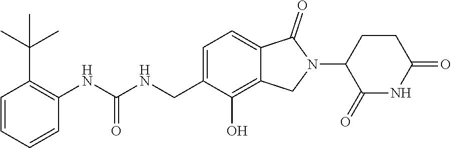 [SMILES: CC(C)(C)c1ccccc1NC(=O)NCc1ccc2c(c1O)CN(C1CCC(=O)NC1=O)C2=O] | 1-(2-(tert-butyl)phenyl)-3-((2-(2,6- dioxopiperidin-3-yl)-4-hydroxy-1- oxoisoindolin-5-yl)methyl)urea | 464.5 |
| IG-29 | 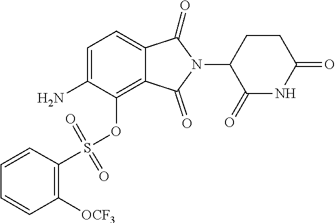 [SMILES: Nc1ccc2c(c1OS(=O)(=O)c1ccccc1OC(F)(F)F)C(=O)N(C1CCC(=O)NC1=O)C2=O] | 5-amino-2-(2,6-dioxopiperidin-3-yl)- 1,3-dioxoisoindolin-4-yl 2- (trifluoromethoxy)benzenesulfonate | 513.4 |
| IG-30 | 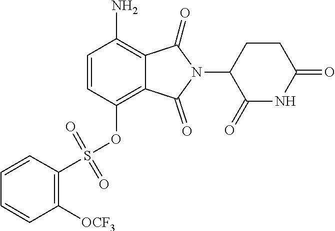 [SMILES: Nc1ccc(OS(=O)(=O)c2ccccc2OC(F)(F)F)c2c1C(=O)N(C1CCC(=O)NC1=O)C2=O] | 7-amino-2-(2,6-dioxopiperidin-3-yl)- 1,3-dioxoisoindolin-4-yl 2- (trifluoromethoxy)benzenesulfonate | 513.4 |
| IG-31 | 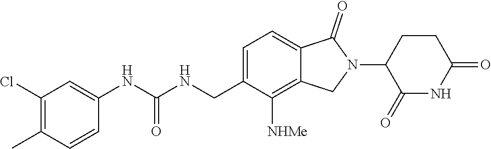 [SMILES: CNc1c(CNC(=O)Nc2ccc(C)c(Cl)c2)ccc2c1CN(C1CCC(=O)NC1=O)C2=O] | 1-(3-chloro-4-methylphenyl)-3-((2- (2,6-dioxopiperidin-3-yl)-4- (methylamino)-1-oxoisoindolin-5- yl)methyl)urea | 469.9 |
| IG-32 | 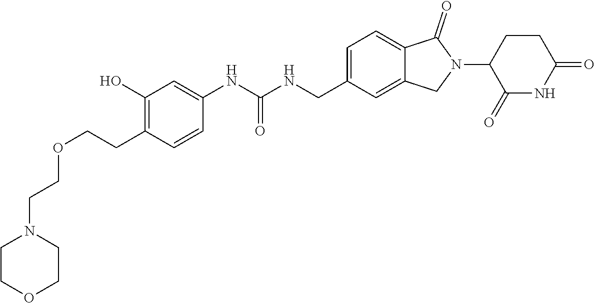 [SMILES: O=C1CCC(N2Cc3cc(CNC(=O)Nc4ccc(CCOCCN5CCOCC5)c(O)c4)ccc3C2=O)C(=O)N1] | 1-((2-(2,6-dioxopiperidin-3-yl)-1- oxoisoindolin-5-yl)methyl)-3-(3- hydroxy-4-(2-(2- morpholinoethoxy)ethyl)phenyl)urea | 565.6 |
| IG-33 | 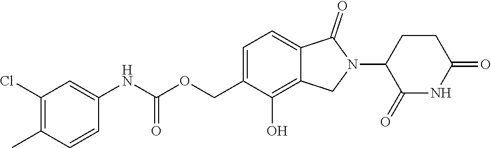 [SMILES: Cc1ccc(NC(=O)OCc2ccc3c(c2O)CN(C2CCC(=O)NC2=O)C3=O)cc1Cl] | (2-(2,6-dioxopiperidin-3-yl)-4- hydroxy-1-oxoisoindolin-5- yl)methyl (3-chloro-4- methylphenyl)carbamate | 457.9 |
| IG-34 | 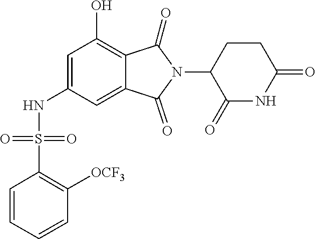 [SMILES: O=C1CCC(N2C(=O)c3cc(NS(=O)(=O)c4ccccc4OC(F)(F)F)cc(O)c3C2=O)C(=O)N1] | N-(2-(2,6-dioxopiperidin-3-yl)-7- hydroxy-1,3-dioxoisoindolin-5-yl)-2- (trifluoromethoxy) benzenesulfonamide | 513.4 |
| IG-35 | 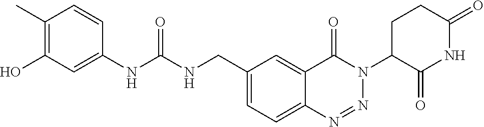 [SMILES: Cc1ccc(NC(=O)NCc2ccc3nnn(C4CCC(=O)NC4=O)c(=O)c3c2)cc1O] | 1-((3-(2,6-dioxopiperidin-3-yl)-4- oxo-3,4- dihydrobenzo[d][1,2,3]triazin-6- yl)methyl)-3-(3-hydroxy-4- methylphenyl)urea | 436.4 |
| IG-36 | 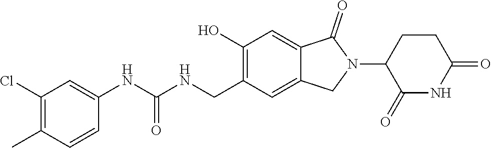 [SMILES: Cc1ccc(NC(=O)NCc2cc3c(cc2O)C(=O)N(C2CCC(=O)NC2=O)C3)cc1Cl] | 1-(3-chloro-4-methylphenyl)-3-((2- (2,6-dioxopiperidin-3-yl)-6-hydroxy- 1-oxoisoindolin-5-yl)methyl)urea | 456.9 |
| IG-37 | 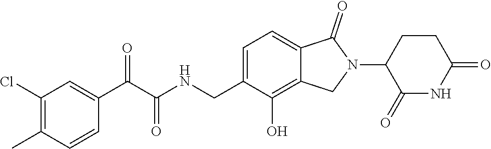 [SMILES: Cc1ccc(C(=O)C(=O)NCc2ccc3c(c2O)CN(C2CCC(=O)NC2=O)C3=O)cc1Cl] | 2-(3-chloro-4-methylphenyl)-N-((2- (2,6-dioxopiperidin-3-yl)-4-hydroxy- 1-oxoisoindolin-5-yl)methyl)-2- oxoacetamide | 469.9 |
| IG-38 | 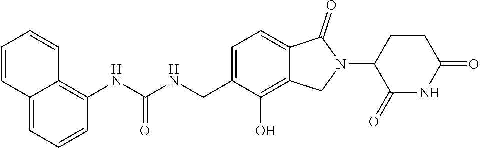 [SMILES: O=C1CCC(N2Cc3c(ccc(CNC(=O)Nc4cccc5ccccc45)c3O)C2=O)C(=O)N1] | 1-((2-(2,6-dioxopiperidin-3-yl)-4- hydroxy-1-oxoisoindolin-5- yl)methyl)-3-(naphthalen-1-yl)urea | 458.5 |
| IG-39 | 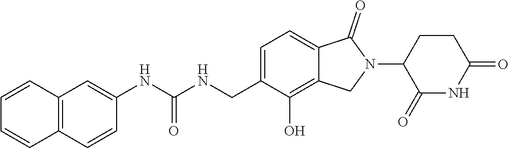 [SMILES: O=C1CCC(N2Cc3c(ccc(CNC(=O)Nc4ccc5ccccc5c4)c3O)C2=O)C(=O)N1] | 1-((2-(2,6-dioxopiperidin-3-yl)-4- hydroxy-1-oxoisoindolin-5- yl)methyl)-3-(naphthalen-2-yl)urea | 458.5 |
| IG-40 | 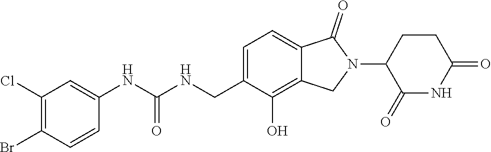 [SMILES: O=C1CCC(N2Cc3c(ccc(CNC(=O)Nc4ccc(Br)c(Cl)c4)c3O)C2=O)C(=O)N1] | 1-(4-bromo-3-chlorophenyl)-3-((2- (2,6-dioxopiperidin-3-yl)-4-hydroxy- 1-oxoisoindolin-5-yl)methyl)urea | 521.8 |
| IG-41 | 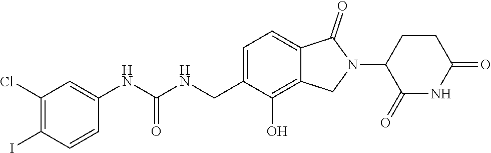 [SMILES: O=C1CCC(N2Cc3c(ccc(CNC(=O)Nc4ccc(I)c(Cl)c4)c3O)C2=O)C(=O)N1] | 1-(3-chloro-4-iodophenyl)-3-((2- (2,6-dioxopiperidin-3-yl)-4-hydroxy- 1-oxoisoindolin-5-yl)methyl)urea | 568.8 |
| IG-42 | 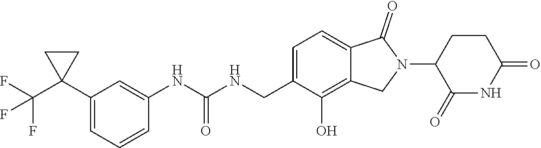 [SMILES: O=C1CCC(N2Cc3c(ccc(CNC(=O)Nc4cccc(C5(C(F)(F)F)CC5)c4)c3O)C2=O)C(=O)N1] | 1-((2-(2,6-dioxopiperidin-3-yl)-4- hydroxy-1-oxoisoindolin-5- yl)methyl)-3-(3-(1- (trifluoromethyl)cyclopropyl)phenyl) urea | 516.5 |
| IG-43 | 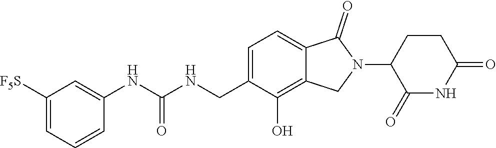 | 1-((2-(2,6-dioxopiperidin-3-yl)-4- hydroxy-1-oxoisoindolin-5- yl)methyl)-3-(3-(pentafluoro-l6- sulfaneyl)phenyl)urea | 534.5 |
| IG-44 | 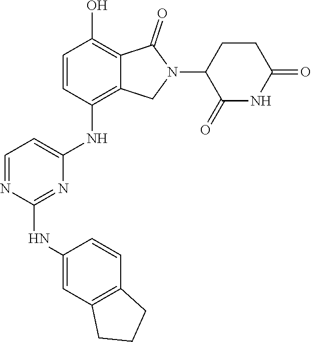 [SMILES: O=C1CCC(N2Cc3c(Nc4ccnc(Nc5ccc6c(c5)CCC6)n4)ccc(O)c3C2=O)C(=O)N1] | 3-(4-((2-((2,3-dihydro-1H-inden-5- yl)amino)pyrimidin-4-yl)amino)-7- hydroxy-1-oxoisoindolin-2- yl)piperidine-2,6-dione | 484.5 |
| IG-45 | 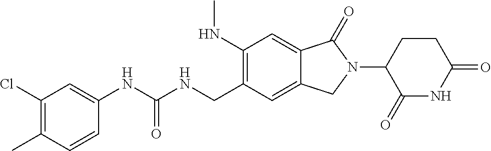 [SMILES: CNc1cc2c(cc1CNC(=O)Nc1ccc(C)c(Cl)c1)CN(C1CCC(=O)NC1=O)C2=O] | 1-(3-chloro-4-methylphenyl)-3-((2- (2,6-dioxopiperidin-3-yl)-6- (methylamino)-1-oxoisoindolin-5- yl)methyl)urea | 469.9 |
| IG-46 | 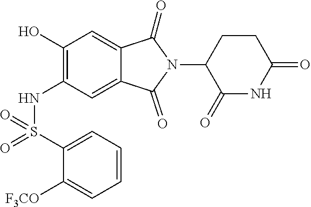 [SMILES: O=C1CCC(N2C(=O)c3cc(O)c(NS(=O)(=O)c4ccccc4OC(F)(F)F)cc3C2=O)C(=O)N1] | N-(2-(2,6-dioxopiperidin-3-yl)-6- hydroxy-1,3-dioxoisoindolin-5-yl)-2- (trifluoromethoxy) benzenesulfonamide | 513.2 |
| IG-47 | 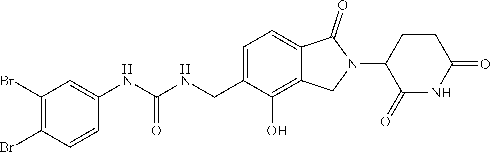 [SMILES: O=C1CCC(N2Cc3c(ccc(CNC(=O)Nc4ccc(Br)c(Br)c4)c3O)C2=O)C(=O)N1] | 1-(3,4-dibromophenyl)-3-((2-(2,6- dioxopiperidin-3-yl)-4-hydroxy-1- oxoisoindolin-5-yl)methyl)urea | 566.2 |
| IG-48 | 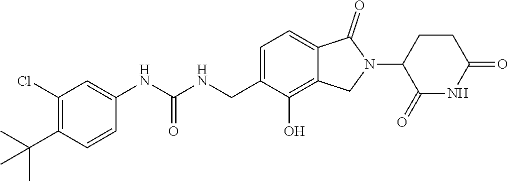 [SMILES: CC(C)(C)c1ccc(NC(=O)NCc2ccc3c(c2O)CN(C2CCC(=O)NC2=O)C3=O)cc1Cl] | 1-(4-(tert-butyl)-3-chlorophenyl)-3- ((2-(2,6-dioxopiperidin-3-yl)-4- hydroxy-1-oxoisoindolin-5- yl)methyl)urea | 499.0 |
| IG-49 | 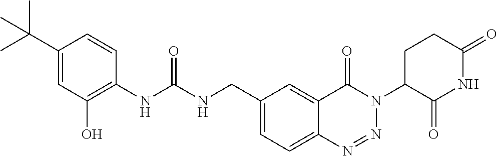 [SMILES: CC(C)(C)c1ccc(NC(=O)NCc2ccc3nnn(C4CCC(=O)NC4=O)c(=O)c3c2)c(O)c1] | 1-(4-(tert-butyl)-2-hydroxyphenyl)- 3-((3-(2,6-dioxopiperidin-3-yl)-4- oxo-3,4- dihydrobenzo[d][1,2,3]triazin-6- yl)methyl)urea | 478.5 |
| IG-50 | 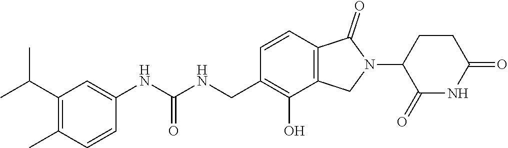 [SMILES: Cc1ccc(NC(=O)NCc2ccc3c(c2O)CN(C2CCC(=O)NC2=O)C3=O)cc1C(C)C] | 1-((2-(2,6-dioxopiperidin-3-yl)-4- hydroxy-1-oxoisoindolin-5- yl)methyl)-3-(3-isopropyl-4- methylphenyl)urea | 464.5 |
| IG-51 | 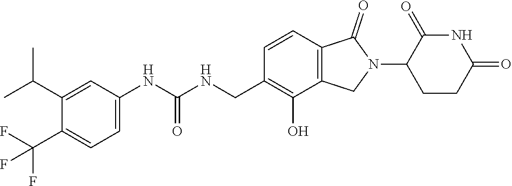 [SMILES: CC(C)c1cc(NC(=O)NCc2ccc3c(c2O)CN(C2CCC(=O)NC2=O)C3=O)ccc1C(F)(F)F] | 1-((2-(2,6-dioxopiperidin-3-yl)-4- hydroxy-1-oxoisoindolin-5- yl)methyl)-3-(3-isopropyl-4- (trifluoromethyl)phenyl)urea | 518.5 |
| IG-52 | 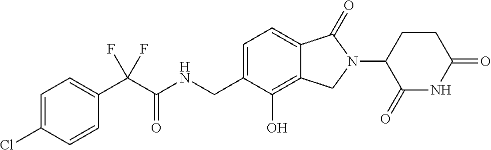 [SMILES: O=C1CCC(N2Cc3c(ccc(CNC(=O)C(F)(F)c4ccc(Cl)cc4)c3O)C2=O)C(=O)N1] | 2-(4-chlorophenyl)-N-((2-(2,6- dioxopiperidin-3-yl)-4-hydroxy-1- oxoisoindolin-5-yl)methyl)-2,2- difluoroacetamide | 447.8 |
| IG-53 | 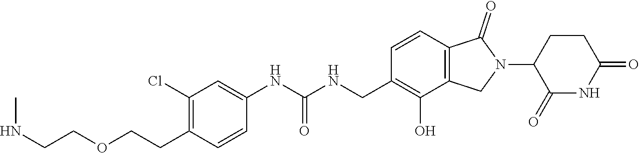 [SMILES: CNCCOCCc1ccc(NC(=O)NCc2ccc3c(c2O)CN(C2CCC(=O)NC2=O)C3=O)cc1Cl] | 1-(3-chloro-4-(2-(2- (methylamino)ethoxy)ethyl)phenyl)- 3-((2-(2,6-dioxopiperidin-3-yl)-4- hydroxy-1-oxoisoindolin-5- yl)methyl)urea | 544.0 |
| IG-54 | 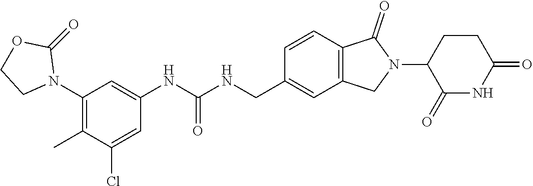 [SMILES: Cc1c(Cl)cc(NC(=O)NCc2ccc3c(c2)CN(C2CCC(=O)NC2=O)C3=O)cc1N1CCOC1=O] | 1-(3-chloro-4-methyl-5-(2- oxooxazolidin-3-yl)phenyl)-3-((2- (2,6-dioxopiperidin-3-yl)-1- oxoisoindolin-5-yl)methyl)urea | 525.9 |
| IG-55 | 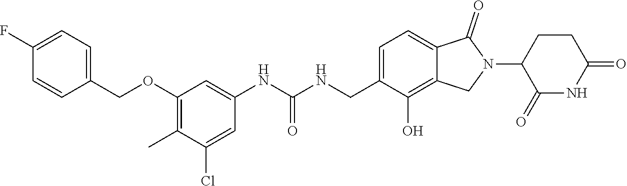 [SMILES: Cc1c(Cl)cc(NC(=O)NCc2ccc3c(c2O)CN(C2CCC(=O)NC2=O)C3=O)cc1OCc1ccc(F)cc1] | 1-(3-chloro-5-((4-fluorobenzyl)oxy)- 4-methylphenyl)-3-((2-(2,6- dioxopiperidin-3-yl)-4-hydroxy-1- oxoisoindolin-5-yl)methyl)urea | 581.0 |
| IG-56 | 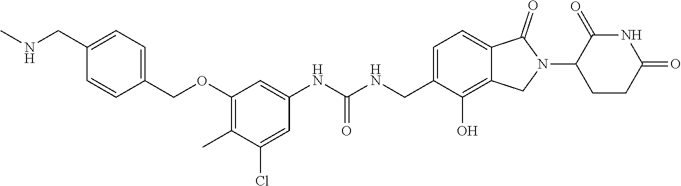 [SMILES: CNCc1ccc(COc2cc(NC(=O)NCc3ccc4c(c3O)CN(C3CCC(=O)NC3=O)C4=O)cc(Cl)c2C)cc1] | 1-(3-chloro-4-methyl-5-((4- ((methylamino)methyl)benzyl)oxy) phenyl)-3-((2-(2,6-dioxopiperidin-3- yl)-4-hydroxy-1-oxoisoindolin-5- yl)methyl)urea | 606.1 |

TABLE 1b
Cell killing by Isoindolinone-glutarimide
(IG) compounds of Table 1a

| | | WSU-DLCL2 | | NCI-N87 |
|---|---|---|---|---|
| IG | CAL51 IC50 | IC50 | SKBR3 IC50 | IC50 |
| No. | (nM) | (nM) | (nM) | (nM) |
| IG-1 | 10.79667 | 1.231333 | 1.766667 | 1.48 |
| IG-2 | >10,000 | >10,000 | >10,000 | |
| IG-3 | 3014 | 66.4 | 77.5 | |
| IG-4 | >10,000 | | | |
| IG-5 | 6856 | | | |
| IG-6 | 7513 | | | |
| IG-7 | 8476 | | | |
| IG-8 | | | | |
| IG-9 | 6493 | | | |
| IG-10 | >10,000 | | | |
| IG-11 | >10,000 | | | |
| IG-12 | 1252 | | | |
| IG-13 | 3035 | | | |
| IG-14 | >10,000 | | | |
| IG-15 | 54.9 | 4.25 | 5.19 | |
| IG-16 | 444 | | | |
| IG-17 | >10,000 | | | |
| IG-18 | >10,000 | | | |
| IG-19 | >10,000 | | | |
| IG-20 | >10,000 | | | |
| IG-21 | | | | |
| IG-22 | | | | |
| IG-23 | | | | |
| IG-24 | 412 | | | |
| IG-25 | 43.2 | | | |
| IG-26 | >10,000 | | | |
| IG-27 | >10,000 | | | |
| IG-28 | 22.5 | | | |
| IG-29 | >10,000 | | | |
| IG-30 | >10,000 | | | |
| IG-31 | >10,000 | | | |
| IG-32 | >10,000 | | | |
| IG-33 | >10,000 | | | |
| IG-34 | >10,000 | | | |
| IG-35 | >10,000 | | | |
| IG-36 | 120.4 | 4.95 | 3.5 | |
| IG-37 | 72.5 | 9.6 | 4.2 | |
| IG-38 | 35.9 | | | |
| IG-39 | | | 14.1 | |
| IG-40 | | | 21.3 | |
| IG-41 | 25 | | 17.1 | |
| IG-42 | 25 | 1.43 | 2.73 | 2.57 |
| IG-43 | 472 | | | |
| IG-44 | >10,000 | | | |
| IG-45 | >10,000 | | | |
| IG-46 | >10,000 | | | |
| IG-47 | 1080 | | | |
| IG-48 | 50.05 | | | |
| IG-49 | | | 41.7 | |
| IG-50 | | 0.75 | 1.38 | 1.63 |
| IG-51 | | 10.2 | 15.7 | |
| IG-52 | | >2,000 | 443 | |
| IG-53 | >30,000 | | | |
| IG-54 | | 6.9 | 14.5 | |

TABLE 1c
Isoindolinone-glutarimide (IG) compounds

| IG | DC₅₀ | DC₅₀ (nM) | Dₘₐₓ (%) | Dₘₐₓ (%) |
|---|---|---|---|---|
| No. | (nM)CAL51 | SKBR3 | CAL51 | SKBR3 |
| IG-1 | 3.24 ± 2.18 | <0.700 ± 0.410 | 101 ± 4.45 | 95.5 ± 4.90 |
| | (n = 3) | (n = 2) | (n = 3) | (n = 2) |
| IG-2 | | | <5% | |
| IG-3 | 345 ± 135 | 185 | 79.4 ± 3.63 | 78.2 |
| | (n = 3) | (n = 1) | (n = 3) | (n = 1) |
| IG-4 | | | 17.3 ± 0.107 | |
| | | | (n = 2) | |
| IG-5 | 1.41E+03 ± 44.8 | | 64.6 ± 2.56 | |
| | (n = 2) | | (n = 2) | |
| IG-6 | 834 ± 125 | | 73.3 ± 9.98 | |
| | (n = 2) | | (n = 2) | |
| IG-7 | 818 ± 164 | | 79.7 ± 5.23 | |
| | (n = 2) | | (n = 2) | |
| IG-8 | | | | |
| IG-9 | 157 ± 46.5 | | 83.8 ± 5.50 | |
| | (n = 2) | | (n = 2) | |
| IG-10 | | | 25.1 ± 3.27 | |
| | | | (n = 2) | |
| IG-11 | | | 42.0 ± 9.54 | |
| | | | (n = 2) | |
| IG-12 | 118 ± 35.0 | 52.2 | 96.8 ± 4.12 | 98.3 |
| | (n = 2) | (n = 1) | (n = 2) | (n = 1) |
| IG-13 | | | 38.8 ± 1.98 | |
| | | | (n = 2) | |
| IG-14 | | | 22.7 ± 3.34 | |
| | | | (n = 2) | |
| IG-15 | 35.8 ± 20.4 | <2.26 ± 0.554 | 79.2 ± 9.10 | 91.1 ± 3.35 |
| | (n = 5) | (n = 2) | (n = 5) | (n = 2) |
| IG-16 | 174 ± 106 | | 91.7 ± 5.22 | |
| | (n = 4) | | (n = 4) | |
| IG-17 | | | Not active | |
| IG-18 | | | Not active | |
| IG-19 | | | Not active | |
| IG-20 | | | Not active | |
| IG-21 | | | | |
| IG-22 | | | | |
| IG-23 | | | | |
| IG-24 | | | | |
| IG-25 | 28.9 | | 89.6 | |
| | (n = 1) | | (n = 1) | |
| IG-26 | | | 22.1 | |
| | | | (n = 1) | |
| IG-27 | | | | |
| IG-28 | 46.9 | 5.20 | 94.2 | 90.2 |
| | (n = 1) | (n = 1) | (n = 1) | (n = 1) |
| IG-29 | | | | |
| IG-30 | | | | |
| IG-31 | | | 18.6 | |
| | | | (n = 1) | |
| IG-32 | | | | |
| IG-33 | | | | |
| IG-34 | | | | |
| IG-35 | | | | |
| IG-36 | 660 ± 997 | 4.62 | 78.5 ± 8.18 | 91.8 |
| | (n = 4) | (n = 1) | (n = 4) | (n = 1) |
| IG-37 | 139 | 7.88 | 70.9 | 95.2 |
| | (n = 1) | (n = 1) | (n = 1) | (n = 1) |
| IG-38 | 113 | 10.6 | 93.7 | 94.1 |
| | (n = 1) | (n = 1) | (n = 1) | (n = 1) |
| IG-39 | 81.4 | 42.7 | 69.4 | 77.4 |
| | (n = 1) | (n = 1) | (n = 1) | (n = 1) |
| IG-40 | | | | |
| IG-41 | | | | |
| IG-42 | <5.41 ± 2.79 | 2.00 | 96.1 ± 5.89 | 88.6 |
| | (n = 2) | (n = 1) | (n = 2) | (n = 1) |
| IG-43 | | | | |
| IG-44 | | | | |
| IG-45 | | | | |
| IG-46 | | | | |
| IG-47 | | | | |
| IG-48 | | | | |
| IG-49 | | | | |
| IG-50 | 6.21 | <0.645 ± 0.113 | 105 | 99.6 ± 0.614 |
| | (n = 1) | (n = 2) | (n = 1) | (n = 2) |
| IG-51 | | | | |
| IG-52 | | | | |
| IG-53 | | | | |
| IG-54 | | | | |

### Linker Units

In certain embodiments, the present disclosure provides branched phenyl maleimide compounds (e.g., compounds of Structure (1)) enable the formation of a covalent bond between an isoindolinone-glutarimide linker compound (JG-L) and an antibody (Ab).

Accordingly, some embodiments provide a compound having the following Structure (I):

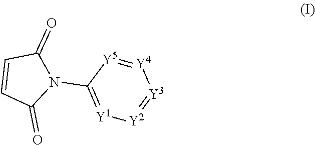

wherein:
one of Y¹, Y², Y³, Y⁴ and Y⁵ is C-L¹-R¹, another one of Y¹, Y², Y³, Y⁴ and Y⁵ is C-L²-R², and the remaining three of Y¹, Y², Y³, Y⁴ and Y⁵ are each independently N, C—R³, or C-L³-R^{3a};
R¹, R², and R^{3a} each independently comprise one or more moieties selected from an amino acid element, a charged element, a heteroalkylene element, a hydrophilic element, a trigger element, an immolative unit, a polar cap, an isoindolinone-glutarimide moiety, and combinations thereof;
   provided that at least one of R¹ and R² comprises a isoindolinone-glutarimide moiety;
each occurrence of R³ is independently selected from the group consisting of hydrogen, deuterium, alkyl, haloalkyl, halo, alkoxy, haloalkoxy, amino, aminyl, amidyl, aldehyde, hydroxyl, cyano, nitro, thiol, carboxy, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide, and a carbohydrate;
R^{4a} and R^{4b} are each independently hydrogen, deuterium, halo, or —S—R^{4c} wherein R^{4c} is substituted or unsubstituted C₆-C₁₀ aryl or substituted or unsubstituted 5-12 membered heteroaryl; and
L¹, L², and L³ are each independently a linker comprising an optionally substituted alkylene, an optionally substituted alkenylene, an optionally substituted alkynylene, an optionally substituted heteroalkylene, an optionally substituted heteroalkenylene, an optionally substituted heteroalkynylene, a heteroatomic linker, an optionally substituted cycloalkylene, an optionally substituted arylene, an optionally substituted heterocyclylene, an optionally substituted heteroarylene, or combinations thereof;
   as a stereoisomer, enantiomer or tautomer thereof or a mixture thereof; or a pharmaceutically acceptable salt, solvate or prodrug thereof.

Certain embodiments provide a compound having the following Structure (Ia):

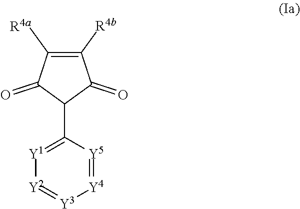

wherein:
one of Y¹, Y², Y³, Y⁴ and Y⁵ is C-L¹-R¹, another one of Y, Y², Y³, Y⁴ and Y⁵ is C-L²-R², and the remaining three of Y¹, Y², Y³, Y⁴ and Y⁵ are each independently N, C—R³, or C-L3-R^{3a};

R¹, R², and R^{3a} each independently comprise one or more moieties selected from an amino acid element, a charged element, a heteroalkylene element, a hydrophilic element, a trigger element, an immolative element, a polar cap, a payload, and combinations thereof; provided that at least one of R¹ and R² comprises a payload;
each occurrence of R³ is independently selected from the group consisting of hydrogen, deuterium, alkyl, haloalkyl, halo, alkoxy, haloalkoxy, amino, aminyl, amidyl, aldehyde, hydroxyl, cyano, nitro, thiol, carboxy, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide, and a carbohydrate;
R^{4a} and R^{4b} are each independently hydrogen, deuterium, halo, or —S—R^{4c} wherein R^{4c} is substituted or unsubstituted C₆-C₁₀ aryl or substituted or unsubstituted 5-12 membered heteroaryl; and
L¹, L², and L³ are each independently direct bond or a linker comprising an optionally substituted alkylene, an optionally substituted alkenylene, an optionally substituted alkynylene, an optionally substituted heteroalkylene, an optionally substituted heteroalkenylene, an optionally substituted heteroalkynylene, a heteroatomic linker, an optionally substituted cycloalkylene, an optionally substituted arylene, an optionally substituted heterocyclylene, an optionally substituted heteroarylene, or combinations thereof;
   as a stereoisomer, enantiomer or tautomer thereof or a mixture thereof; or a pharmaceutically acceptable salt, solvate or prodrug thereof.

In some embodiments, R^{4a} and R^{4b} are both hydrogen. In some embodiments, R^{4a} is halo or R^{4b} is halo. In some embodiments, R^{4a}, R^{4b}, or both have one of the following structures:

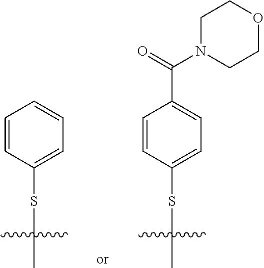 [SMILES: *Sc1ccccc1 ; *Sc1ccc(C(=O)N2CCOCC2)cc1]

In some embodiments, R¹, R², and/or R^{3a} comprises elements selected from an amino acid element, a charged element, a heteroalkylene element, a hydrophilic element, a trigger element, an immolative element, a polar cap, a payload, and combinations thereof. It is understood that these elements can be connected in any order and be connected in a linear manner or via a branched connection. In some embodiments, R¹, R², and/or R^{3a} comprises multiple occurrences of an element (e.g., two or more heteroalkylene elements, two or more hydrophilic elements, two or more polar caps, etc.).

In some embodiments, R¹, R², or R^{3a} comprises a branch point as part of an amino acid element (e.g., lysine) wherein additional elements are attached via an epsilon amine of the lysine and other additional elements are linked to the amino acid element via one or more peptide bonds to the alpha carbon of a lysine. A similar motif could be utilized with a glutamic acid of an amino acid element. In some embodiments, an amino acid element comprises one of the following structures:

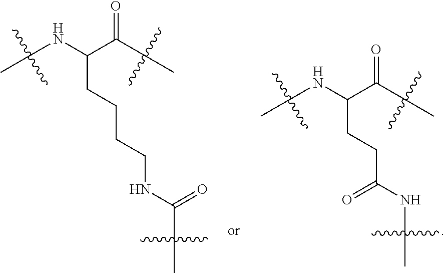 [SMILES: *NC(CCCCNC(*)=O)C(*)=O ; *NC(=O)CCC(N*)C(*)=O]

In some embodiments, a compound of Structure (I) comprises one of the following structures:

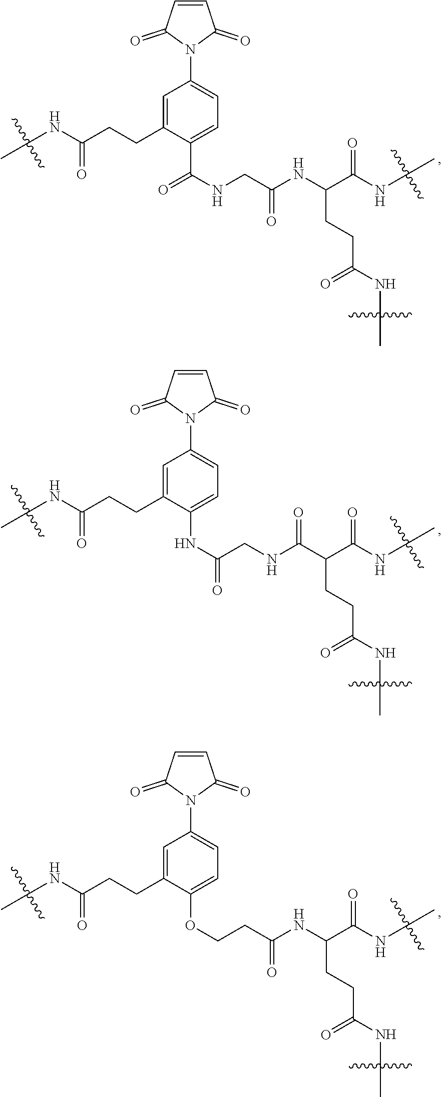 [SMILES: *NC(=O)CCc1cc(N2C(=O)C=CC2=O)ccc1C(=O)NCC(=O)NC(CCC(=O)N*)C(=O)N* ; *NC(=O)CCc1cc(N2C(=O)C=CC2=O)ccc1NC(=O)CNC(=O)C(CCC(=O)N*)C(=O)N* ; *NC(=O)CCc1cc(N2C(=O)C=CC2=O)ccc1OCCC(=O)NC(CCC(=O)N*)C(=O)N*]

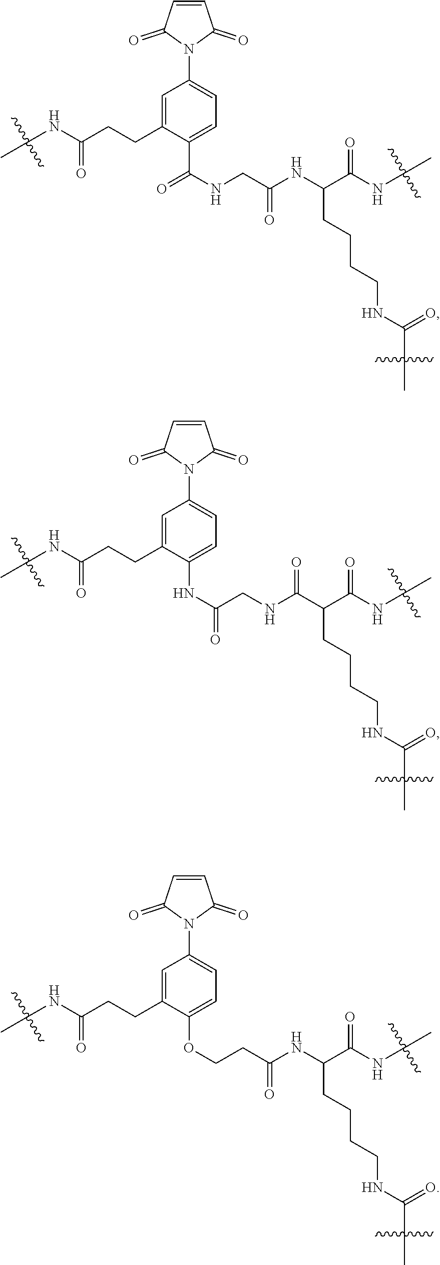 [SMILES: *NC(=O)CCc1cc(N2C(=O)C=CC2=O)ccc1C(=O)NCC(=O)NC(CCCCNC(*)=O)C(=O)N* ; *NC(=O)CCc1cc(N2C(=O)C=CC2=O)ccc1NC(=O)CNC(=O)C(CCCCNC(*)=O)C(=O)N* ; *NC(=O)CCc1cc(N2C(=O)C=CC2=O)ccc1OCCC(=O)NC(CCCCNC(*)=O)C(=O)N*]

In certain embodiments, R¹ has the following structure:

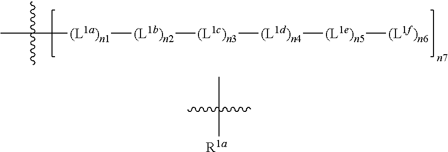

wherein:
L^{1a} is an amino acid element;
L^{1b} is a charged element;
L^{1c} is a heteroalkylene element;
L^{1d} is a hydrophilic element;
L^{1e} is a trigger element; and
L^{1f} is an immolative unit;
   wherein one or more occurrence of L^{1a}, L^{1b}, L^{1c}, L^{1d}, L^{1e}, and L^{1f} optionally joins with one or more of another of L^{1a}, L^{1b}, L^{1c}, L^{1d}, L^{1e}, and L^{1f} to form one or more ring;
each occurrence of n1, n2, n3, n4, n5, and n6 is independently an integer from 0-3, provided that n1+n2+n3+n4+n5+n6=1 or more;
n7 is 1, 2, 3, 4, 5, or 6; and
R^{1a} is a isoindolinone-glutarimide moiety that is covalently bound to one occurrence of L^{1a}, L^{1b}, L^{1c}, L^{1d}, L^{1e}, or L^{1f} and the isoindolinone-glutarimide moiety is optionally substituted with a polar cap.

In some embodiments, n7 is 1, 2, or 3. In some embodiments, n7 is 1 or 2. In some embodiments, n7 is 1.

In some embodiments, R¹ has the following structure:

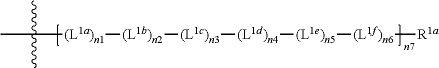

wherein:
L^{1a} is an amino acid element;
L^{1b} is a charged element;
L^{1c} is a heteroalkylene element;
L^{1d} is a hydrophilic element;
L^{1e} is a trigger element; and
L^{1f} is an immolative unit;
   wherein one or more occurrence of L^{1a}, L^{1b}, L^{1c}, L^{1d}, L^{1e}, and L^{1f} optionally joins with one or more of another of L^{1a}, L^{1b}, L^{1c}, L^{1d}, L^{1e}, and L^{1f} to form one or more ring;
each occurrence of n1, n2, n3, n4, n5, and n6 is independently an integer from 0-3, provided that n1+n2+n3+n4+n5+n6=1 or more;
n7 is 1, 2, 3, 4, 5, or 6; and
R^{1a} is a isoindolinone-glutarimide moiety optionally substituted with a polar cap.

In more embodiments, R² has the following structure:

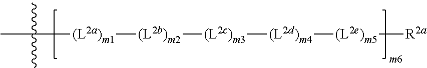

wherein:
L^{2a} is an amino acid element;
L^{2b} is a charged element;
L^{2c} is a heteroalkylene element;
L^{2d} is a hydrophilic element;
L^{2e} is a trigger element;
each occurrence of m1, m2, m3, m4, and m5 is independently an integer from 0-3, provided that m1+m2+m3+m4+m5=1 or more;
m6 is 1,2,3,4, or 5; and
R^{2a} is hydrogen, alkyl, a isoindolinone-glutarimide moiety, or a polar cap.

In some embodiments, m6 is 1, 2, or 3. In some embodiments, m6 is 1 or 2. In some embodiments, m6 is 1.

In still more embodiments, R^{3a} has the following structure:

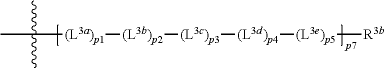

wherein:
L^{3a} is an amino acid element;
L^{3b} is a charged element;
L^{3c} is a heteroalkylene element;
L^{3d} is a hydrophilic element;
L^{3e} is a trigger element;
each occurrence of p1, p2, p3, p4, and p5 is independently an integer from 0-3, provided that p1+p2+p3+p4+p5=1 or more;
p6 is 1, 2, 3, 4, or 5; and
R^{3b} is hydrogen, alkyl, or a polar cap.

In some embodiments, p6 is 1, 2, or 3. In some embodiments, p6 is 1 or 2. In some embodiments, p6 is 1.

In certain embodiments, n7 is 1 and each of n1 through n6 are 1. In some embodiments, n7 is 1 and each of n1 through n4 are 0, n5 is 1, and n6 is 1. In certain embodiments, n7 is 1 and n1 is 0, n2 is 0, n3 is 1, n4 is 0, n5 is 1, and n6 is 1. In certain embodiments, n7 is 1 and n1 is 1, n2 is 0, n3 is 1, n4 is 0, n5 is 1, and n6 is 1. In certain embodiments, n7 is 1 and n1 is 1, n2 is 1, n3 is 1, n4 is 0, n5 is 1, and n6 is 1. In certain embodiments, n7 is 1 and n1 is 1, n2 is 1, n3 is 1, n4 is 1, n5 is 1, and n6 is 1. In certain embodiments, n7 is 1 and n1 is 1, n2 is 0, n3 is 1, n4 is 0, n5 is 1, and n6 is 1. In certain embodiments, n7 is 2. In certain embodiments, n7 is 3.

In some embodiments, m6 is 1 and each of m1 through m5 are 1. In some embodiments, m6 is 1, m1 is 1, m2 is 0, m3 is 0, m4 is 1, and m5 is 0. In some embodiments, m6 is 1, m1 is 1, m2 is 1, m3 is 0, m4 is 1, and m5 is 0. In some embodiments, m6 is 1, m1 is 1, m2 is 0, m3 is 1, m4 is 1, and m5 is 0. In some embodiments, m6 is 1, m1 is 1, m2 is 0, m3 is 0, m4 is 1, and m5 is 1. In some embodiments, m6 is 2. In certain embodiments, m6 is 3.

In some embodiments, p6 is 1 and each of p1 through p5 is 1. In certain embodiments, p6 is 1, p1 is 1, p2 is 0, p3 is 0, p4 is 1, and p5 is 0. In some embodiments, p6 is 1, p1 is 1, p2 is 1, p3 is 0, p4 is 1, and p5 is 0. In some embodiments, p6 is 1, p1 is 1, p2 is 0, p3 is 1, p4 is 1, and p5 is 0 In some embodiments, p6 is 1, p1 is 1, p2 is 0, p3 is 0, p4 is 1, and p5 is 1. In some embodiments, p6 is 2 and at least one occurrence of p1 is 1, p2 is 1, p3 is 0, p4 is 1, and p5 is 0. In some embodiments, p6 is 2. In certain embodiments, p6 is 3.

In some embodiments, an amino acid element comprises one or more amino acids selected from the group consisting of glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, sarcosine, and beta-alanine.

In certain embodiments, an amino acid element is selected from the group consisting of glycine, sarcosine, beta-alanine, and glutamic acid.

In some embodiments, an amino acid element comprises a dipeptide, a tripeptide, a tetrapeptide, or a pentapeptide.

In more embodiments, an amino acid element has one of the following structures:

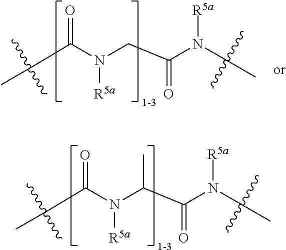

wherein:
each occurrence of R^{5a} is independently hydrogen, alkyl, hydroxyalkyl, or alkoxyalkyl.

In some embodiments, a charged element comprises moieties with a negative charge at about pH 7.4 (i.e., a range from 6.3 to 8.5). In certain embodiments, a charged element comprises moieties with a positive charge at about pH 7.4 (i.e., a range from 6.3 to 8.5).

In some embodiments, a charged element comprises one or more charged amino acid, one or more carboxylic acid, one or more sulfonic acid, one or more sulfonamide, one or more sulfate, one or more phosphate, one or more quaternary amine, one or more sulfamide, one or more sulfinimide, or combinations thereof.

In certain embodiments, a charged amino acid is aspartic acid, glutamic acid, histidine, lysine, or arginine.

In some embodiments, R¹, R², or R^{3a} comprises a non-cleavable linker (e.g., a linker, or segment thereof, that does not include a trigger element or immolative unit).

In some embodiments, R¹, R², or R^{3a} comprises one of the following structures:

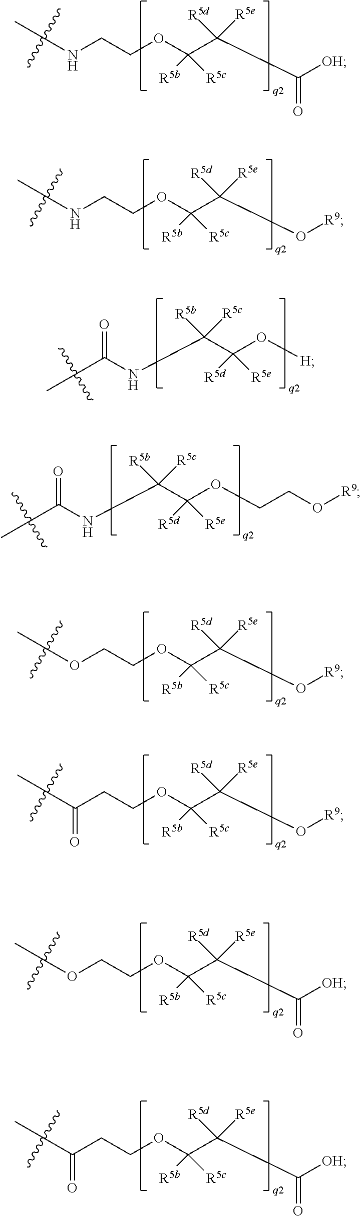

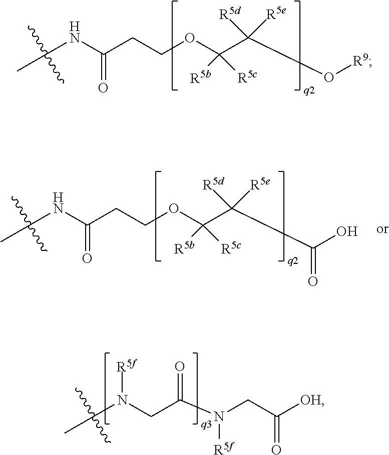

wherein:
each occurrence of R^{5b}, R^{5c}, R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide;
each occurrence of R^{5f} is independently hydrogen, alkyl, hydroxyalkyl, or alkoxyalkyl;
each occurrence of R⁹ is independently hydrogen or alkyl;
each occurrence of q2 is independently an integer from 1-25; and
each occurrence of q3 is independently an integer from 5-15.

In some embodiments, a hydrophilic element comprises polyethylene glycol, polysarcosine, cyclodextrin, c-glycosides, or combinations thereof. In some embodiments, a hydrophilic element comprises one of the following structures:

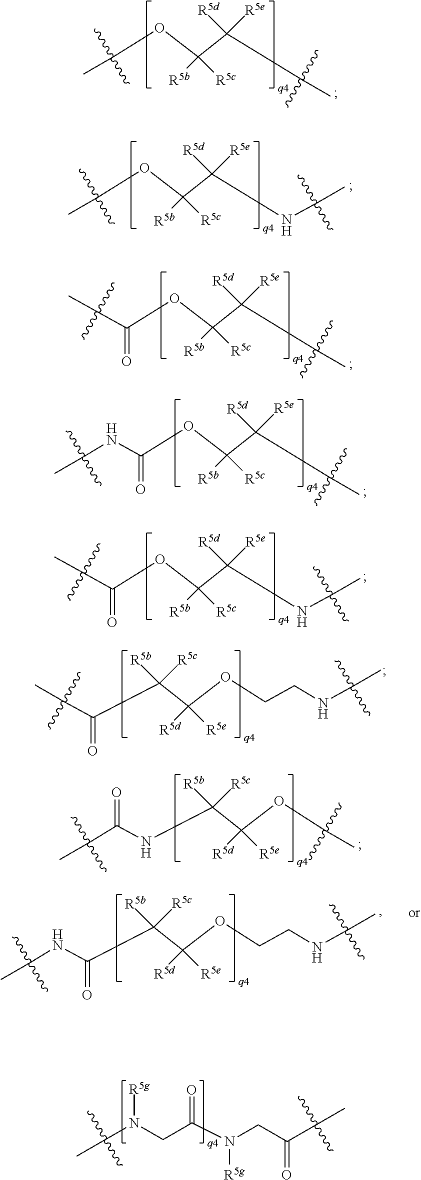

wherein:
each occurrence of R^{5b}, R^{5c} R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
each occurrence of R^{5g} is independently hydrogen, alkyl, hydroxyalkyl, or alkoxyalkyl; and
each occurrence of q4 is, independently an integer from 1-24.

In some embodiments, a hydrophilic element comprises one of the following structures:

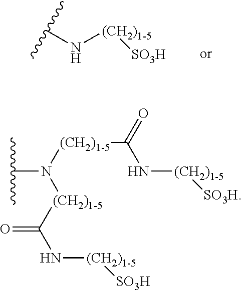 [SMILES: *NCS(=O)(=O)O ; *N(CC(=O)NCS(=O)(=O)O)CC(=O)NCS(=O)(=O)O]

In some embodiments, a hydrophilic element comprises one of the following structures:

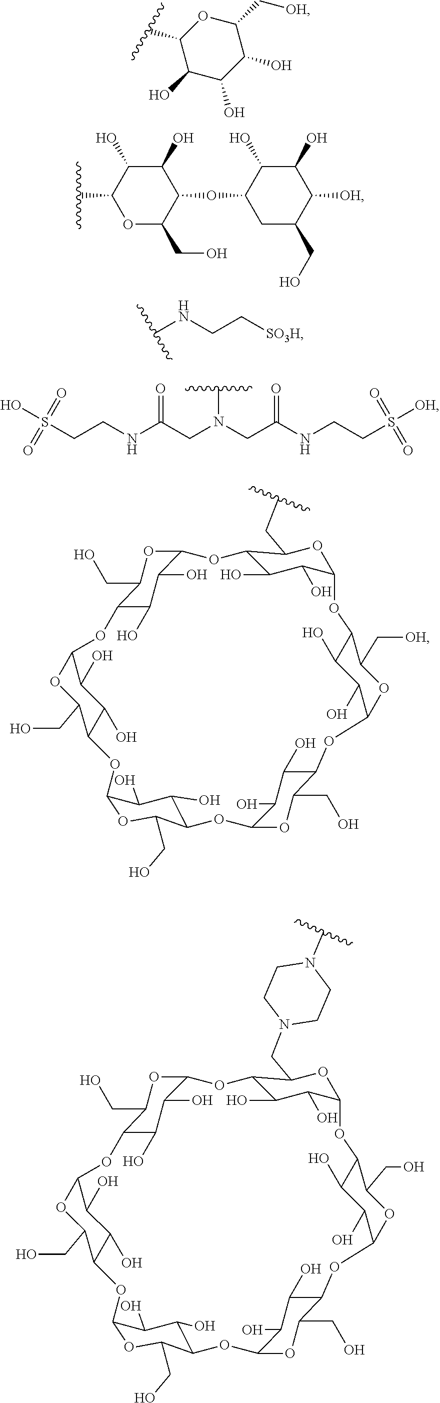

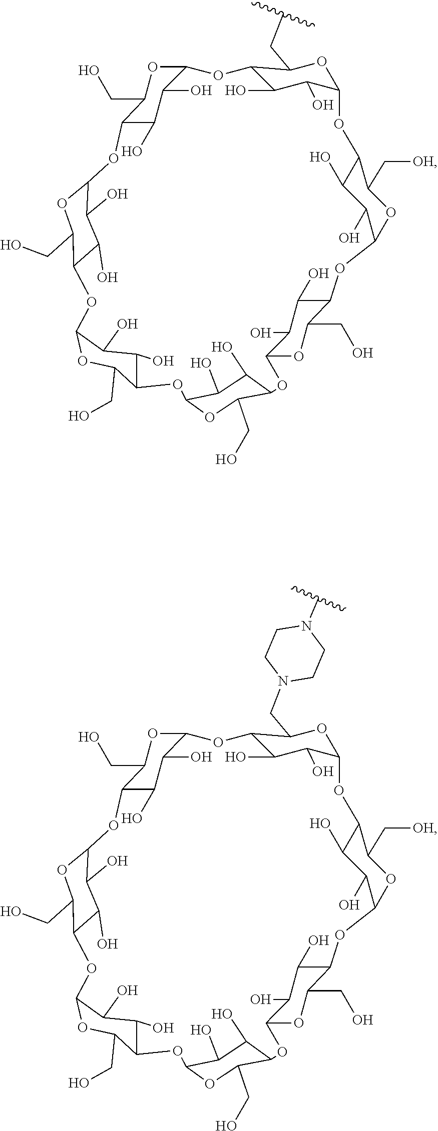 [SMILES: *CC1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O ; *N1CCN(CC2OC3OC4C(CO)OC(OC5C(CO)OC(OC6C(CO)OC(OC7C(CO)OC(OC8C(CO)OC(OC9C(CO)OC(OC2C(O)C3O)C(O)C9O)C(O)C8O)C(O)C7O)C(O)C6O)C(O)C5O)C(O)C4O)CC1]

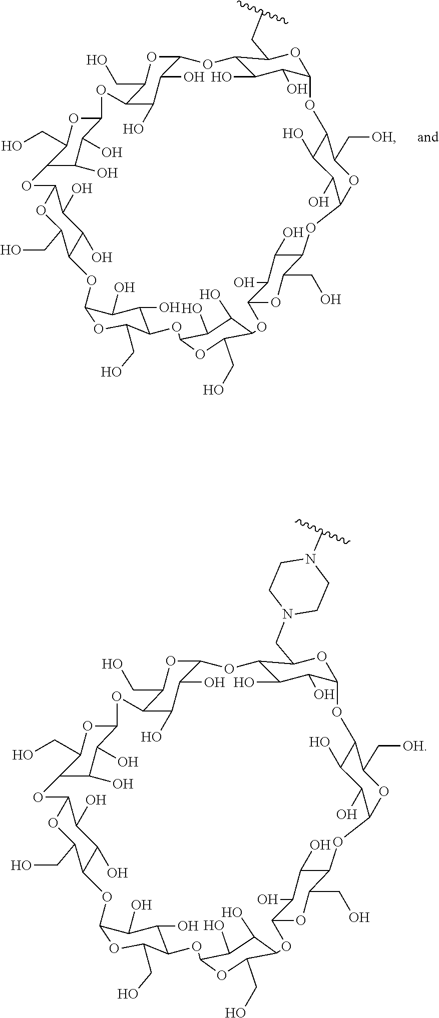 [SMILES: *CC1OC2OC3C(CO)OC(OC4C(CO)OC(OC5C(CO)OC(OC6C(CO)OC(OC7C(CO)OC(OC8C(CO)OC(OC9C(CO)OC(OC1C(O)C2O)C(O)C9O)C(O)C8O)C(O)C7O)C(O)C6O)C(O)C5O)C(O)C4O)C(O)C3O ; *N1CCN(CC2OC3OC4C(CO)OC(OC5C(CO)OC(OC6C(CO)OC(OC7C(CO)OC(OC8C(CO)OC(OC9C(CO)OC(OC%10C(CO)OC(OC2C(O)C3O)C(O)C%10O)C(O)C9O)C(O)C8O)C(O)C7O)C(O)C6O)C(O)C5O)C(O)C4O)CC1]

In some embodiments, a hydrophilic element has one of the following structures:

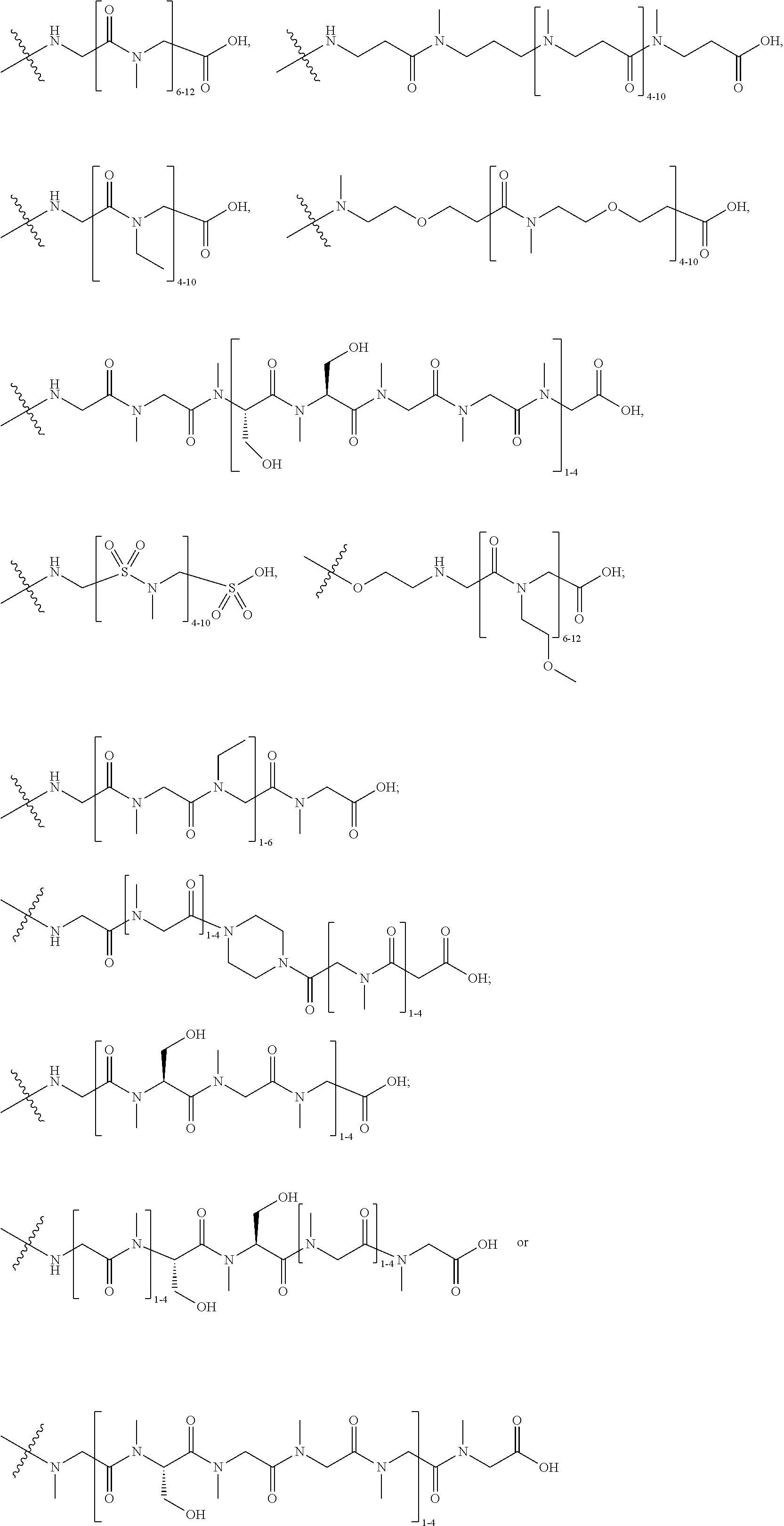

In some embodiments, a hydrophilic element has the following structure:

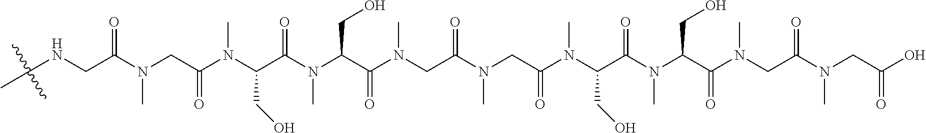 [SMILES: *NCC(=O)N(C)CC(=O)N(C)[C@@H](CO)C(=O)N(C)[C@@H](CO)C(=O)N(C)CC(=O)N(C)CC(=O)N(C)[C@@H](CO)C(=O)N(C)[C@@H](CO)C(=O)N(C)CC(=O)N(C)CC(=O)O]

In some embodiments, a hydrophilic element has one of the following structures:

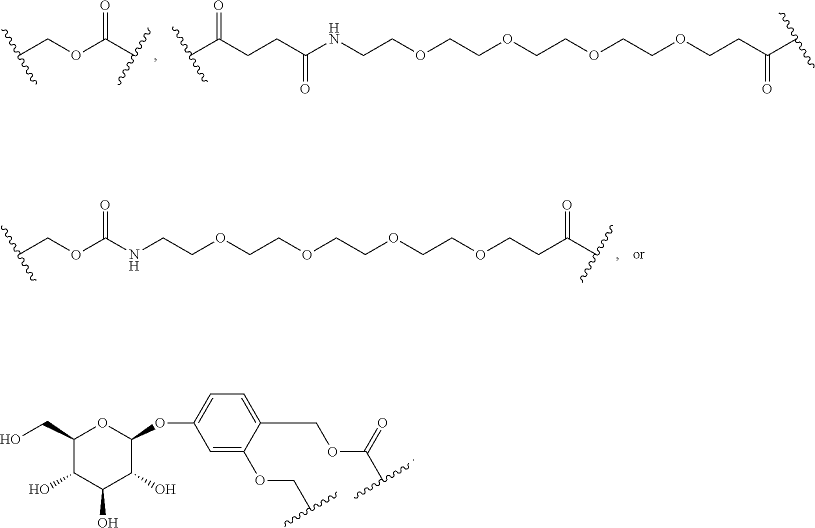 [SMILES: *COC(*)=O ; *C(=O)CCOCCOCCOCCOCCNC(=O)CCC(*)=O ; *COC(=O)NCCOCCOCCOCCOCCC(*)=O ; *COc1cc(O[C@@H]2O[C@H](CO)[C@@H](O)[C@H](O)[C@H]2O)ccc1COC(*)=O]

In some embodiments, a hydrophilic element has one of the following structures:

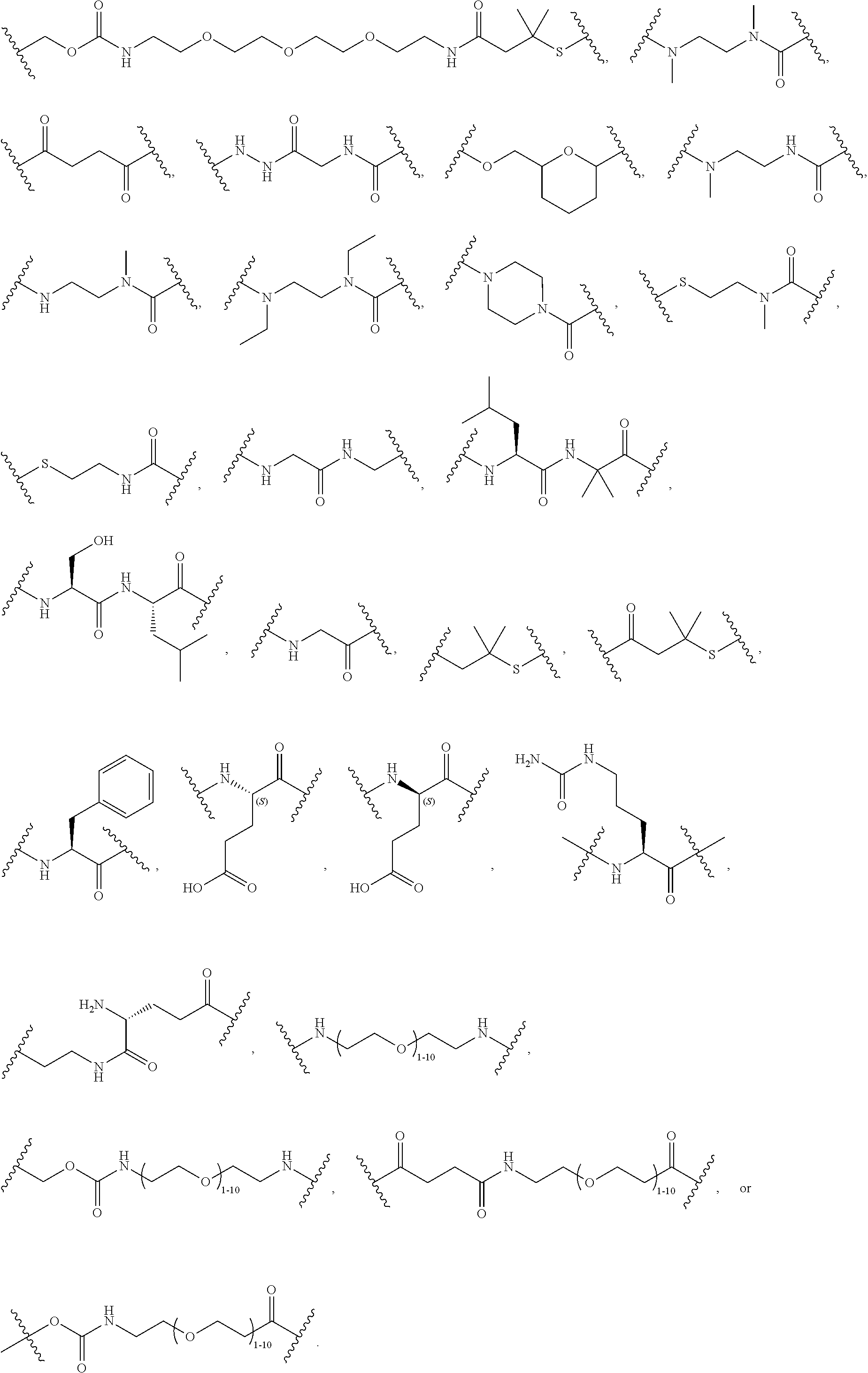

In some embodiments, a hydrophilic element comprises a polysarcosine. In some embodiments, a hydrophilic element is a polysarcosine comprising the following structure:

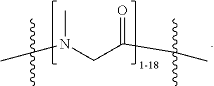

In some embodiments, a hydrophilic element is a polysarcosine with one of the following structures:

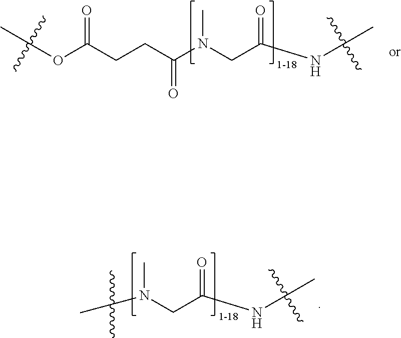

In some embodiments, a hydrophilic element has a molecular weight greater than 150 g/mole, greater than 200 g/mole, greater than 300 g/mole, greater than 400 g/mole, greater than 500 g/mole, greater than 600 g/mole, greater than 700 g/mole, greater than 800 g/mole, greater than 900 g/mole, or greater than 1000 g/mole. In some embodiments, a hydrophilic element has a molecular weight less than 150 g/mole, less than 200 g/mole, less than 300 g/mole, less than 400 g/mole, less than 500 g/mole, less than 600 g/mole, less than 700 g/mole, less than 800 g/mole, less than 900 g/mole, or less than 1000 g/mole.

In certain embodiments, L¹ is alkylene. In some embodiments, L¹ is C₁-C₆ alkylene. In certain embodiments, L² is alkylene. In some embodiments, L² is C₁-C₆ alkylene. In certain embodiments, L³ is alkylene. In some embodiments, L³ is C₁-C₆ alkylene.

In certain embodiments, L¹ is heteroalkylene. In some embodiments, L¹ is C₁-C₆ heteroalkylene (i.e., contains from 1-6 carbon atoms and one or more heteroatoms). In certain embodiments, L² is heteroalkylene. In some embodiments, L² is C₁-C₆ heteroalkylene. In certain embodiments, L³ is heteroalkylene. In some embodiments, L³ is C₁-C₆ heteroalkylene.

In more embodiments, L¹, L², or L³ are C₁-C₆ heteroalkylene and contain heteroatoms selected form O and N. In some embodiments, L³ is a direct bond.

In more embodiments, L¹, L², or L³ have one of the following structures:

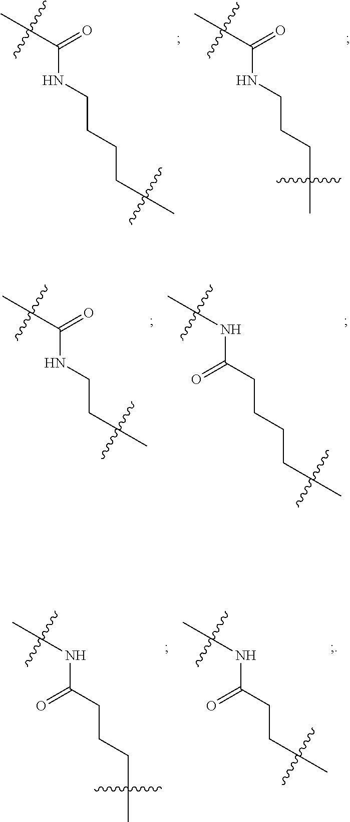 [SMILES: *CCCCNC(*)=O ; *CCCNC(*)=O ; *CCNC(*)=O ; *CCCCC(=O)N* ; *CCCC(=O)N* ; *CCC(=O)N*]

In some embodiments, a trigger element comprises a dipeptide, a tripeptide, a tetrapeptide, a pentapeptide, a glucuronide, a disulfide, a phosphate, a diphosphate, a triphosphate, a hydrazone, or combinations thereof. In some other embodiments, a trigger element comprises beta-glucuronic acid. In certain embodiments, a trigger element comprises a dipeptide, a tripeptide, a tetrapeptide, or a pentapeptide. In some embodiments, a trigger element comprises two or more amino acids selected from the group consisting of valine, citrulline, alanine, glycine, phenylalanine, lysine, or combinations thereof. In certain embodiments, a trigger element comprises a sequence of amino acids selected from the group consisting of valine-citrulline, valine-alanine, glycine-glycine-phenylalanine-glycine, and combinations thereof. In some embodiments, a trigger element comprises one of the following structures, including combinations thereof:

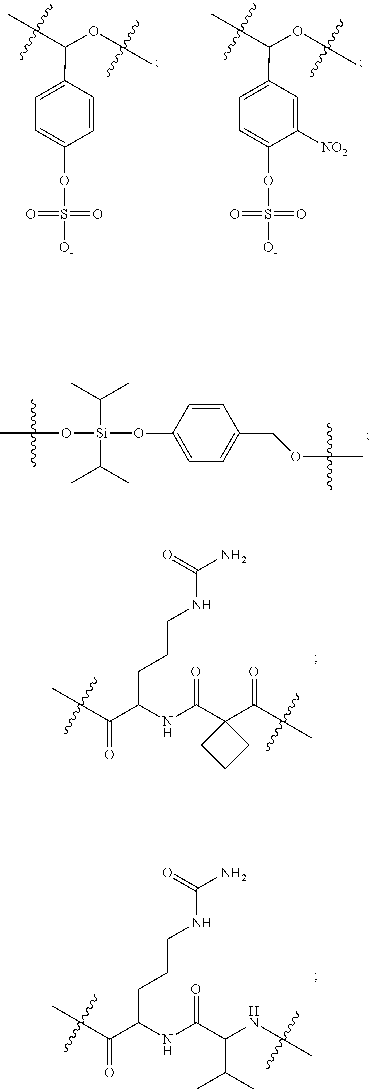

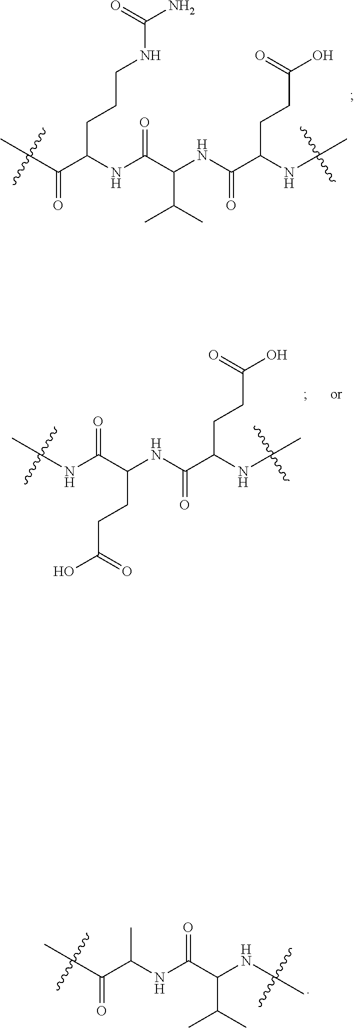 [SMILES: *NC(CCC(=O)O)C(=O)NC(C(=O)NC(CCCNC(N)=O)C(*)=O)C(C)C ; *NC(=O)C(CCC(=O)O)NC(=O)C(CCC(=O)O)N* ; *NC(C(=O)NC(C)C(*)=O)C(C)C]

In some embodiments, a trigger element has a molecular weight greater than 150 g/mole, greater than 200 g/mole, greater than 300 g/mole, greater than 400 g/mole, greater than 500 g/mole, greater than 600 g/mole, greater than 700 g/mole, greater than 800 g/mole, greater than 900 g/mole, or greater than 1000 g/mole. In some embodiments, a trigger element has a molecular weight less than 150 g/mole, less than 200 g/mole, less than 300 g/mole, less than 400 g/mole, less than 500 g/mole, less than 600 g/mole, less than 700 g/mole, less than 800 g/mole, less than 900 g/mole, or less than 1000 g/mole.

In some embodiments, a trigger element has the following structure:

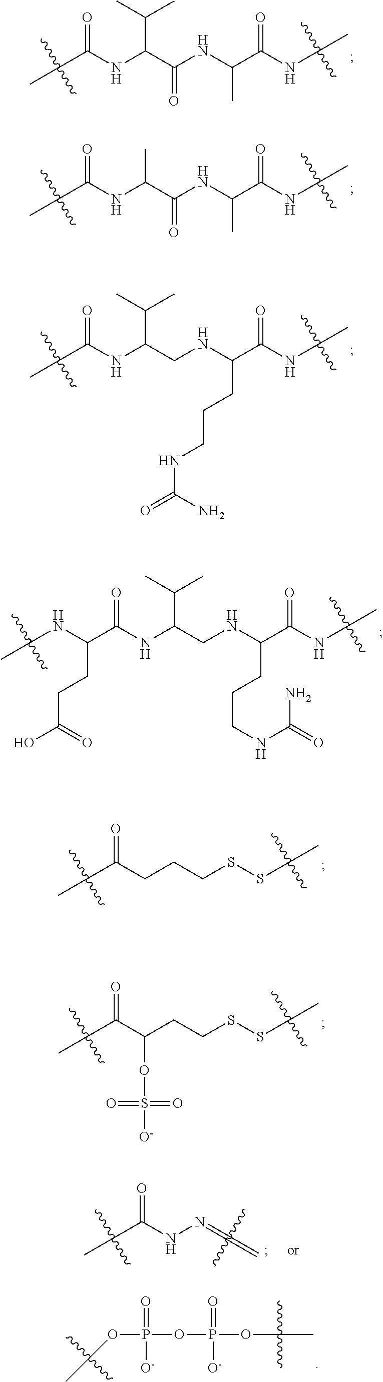

In some embodiments, a trigger element is specifically cleaved by an enzyme. For example, a trigger element can be cleaved by a lysosomal enzyme. A trigger element can be peptide-based or can include peptidic regions that can act as substrates for enzymes. Peptide based trigger elements can be more stable in plasma and extracellular milieu than chemically labile linkers.

Exemplary disulfide-containing trigger elements can include the following structures:

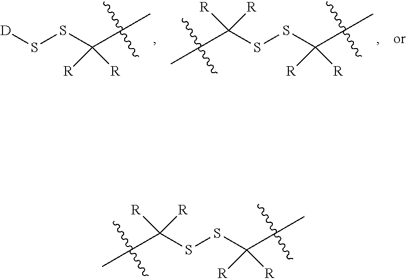

wherein D is a isoindolinone-glutarimide moiety and R is independently selected at each occurrence from, for example, hydrogen or C₁-C₆ alkyl. Increasing steric hindrance adjacent to the disulfide bond can increase the stability of the linker. The above structures can result in increased in vivo stability when one or more R groups is selected from a lower alkyl, such as methyl.

Peptide bonds can have good serum stability, as lysosomal proteolytic enzymes can have very low activity in blood due to endogenous inhibitors and the unfavorably high pH value of blood compared to lysosomes. Release of a isoindolinone-glutarimide moiety from conjugate of Structure (II) can occur due to the action of lysosomal proteases, e.g., cathepsin and plasmin. These proteases can be present at elevated levels in certain tumor tissues. A trigger element can be cleavable by a lysosomal enzyme. The lysosomal enzyme can be, for example, cathepsin B, β-glucuronidase, or β-galactosidase.

A cleavable peptide of a trigger element can be selected from tetrapeptides such as Gly-Phe-Leu-Gly, Ala-Leu-Ala-Leu, tripeptides such as Glu-Val-Cit, or dipeptides such as Val-Cit, Val-Ala, Ala-Ala, and Phe-Lys. Dipeptides can have lower hydrophobicity compared to longer peptides.

In some embodiments, a trigger element may be a single amino acid residue. In some embodiments, the trigger element comprises asparagine, Asn as a legumain cleavable element (Miller, J. T. et al (2021) Bioconjugate Chem. 32(4):842-858

Enzymatically cleavable trigger elements be combined with an immolative unit and provide additional spatial separation between a isoindolinone-glutarimide moiety and the site of enzymatic cleavage. The direct attachment of isoindolinone-glutarimide moiety to a peptidic trigger element can result in proteolytic release of a isoindolinone-glutarimide moiety or of an amino acid adduct of a isoindolinone-glutarimide moiety thereby impairing its activity. The use of an immolative unit can allow for the release of the fully active, chemically unmodified isoindolinone-glutarimide moiety upon amide bond hydrolysis.

A trigger element can contain a chemically labile group such as hydrazone and/or disulfide groups. A trigger element comprising chemically labile group or groups can exploit differential properties between the plasma and some cytoplasmic compartments. The intracellular conditions that can facilitate release of a isoindolinone-glutarimide moiety for hydrazone containing trigger elements can be the acidic environment of endosomes and lysosomes, while the disulfide containing trigger elements can be reduced in the cytosol, which can contain high thiol concentrations, e.g., glutathione. The plasma stability of a trigger element containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release a isoindolinone-glutarimide moiety once the conjugate of Structure (II) is internalized into mildly acidic endosomal (pH 5.0-6.5) and lysosomal (pH 4.5-5.0) compartments of the cell. This pH dependent release mechanism can be associated with non-specific release of a isoindolinone-glutarimide moiety. To increase the stability of a hydrazone group of a trigger element, a trigger element can be varied by chemical modification, e.g., substitution, allowing tuning to achieve more efficient release in the lysosome with a minimized loss in circulation.

In some embodiments, a trigger element comprises a hydrazone moiety having one of the following structures:

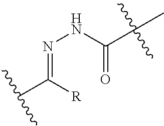

wherein R is selected from C₁-C₆ alkyl, aryl, and —O—C₁-C₆ alkyl.

Hydrazone-containing trigger elements can contain additional cleavage sites, such as additional acid-labile cleavage sites and/or enzymatically labile cleavage sites (e.g., a disulfide). Conjugates and compounds including exemplary hydrazone-containing trigger elements can include, for example, the following structure:

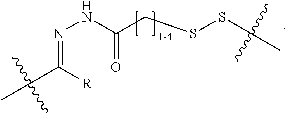

wherein R is selected from C₁-C₆ alkyl, aryl, and —O—C₁-C₆ alkyl.

Other acid-labile groups that can be included in trigger elements include cis-aconityl-containing linkers. cis-Aconityl chemistry can use a carboxylic acid juxtaposed to an amide bond to accelerate amide hydrolysis under acidic conditions.

Trigger elements can also include a disulfide group. Disulfides can be thermodynamically stable at physiological pH and release a isoindolinone-glutarimide moiety upon internalization of the conjugate of Structure (II) into cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing trigger element can be reasonably stable in circulation, selectively releasing a isoindolinone-glutarimide moiety in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing trigger element can be enhanced by chemical modification of a trigger element, e.g., use of steric hindrance adjacent to the disulfide bond.

A trigger element can also be a β-glucuronic acid-based linker. Facile release of a isoindolinone-glutarimide moiety, can be realized through cleavage of the β-glucuronide glycosidic bond by the lysosomal enzyme β-glucuronidase. This enzyme can be abundantly present within lysosomes and can be overexpressed in some tumor types, while the enzyme activity outside cells can be low. β-Glucuronic acid-based linkers can be used to circumvent the tendency of a conjugate to undergo aggregation due to the hydrophilic nature of β-glucuronides. In some embodiments, a trigger element comprises a β-glucuronic acid.

The following scheme depicts the release of a isoindolinone-glutarimide moiety (D) from a conjugate of Structure (II) containing a β-glucuronic acid-based trigger element:

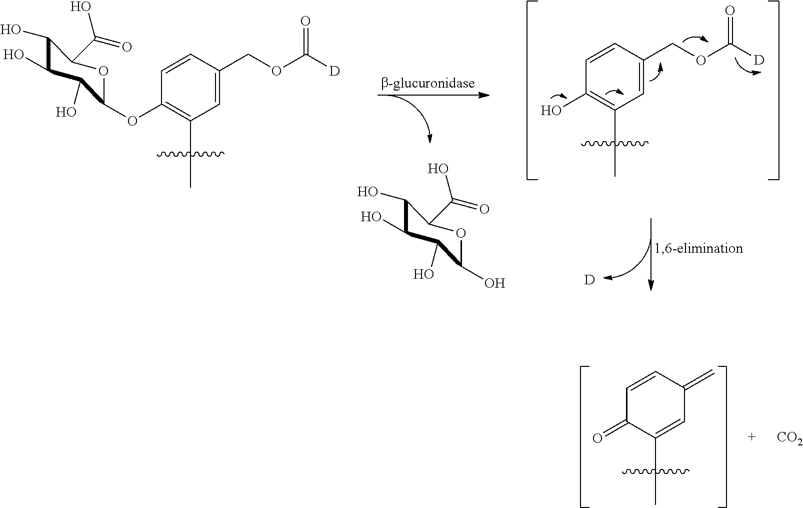

A variety of cleavable β-glucuronic acid-based linkers useful for linking drugs such as auristatins, camptothecin analogues, doxorubicin analogues, CBI minor-groove binders, and psymberin to antibodies have been described. Accordingly, these β-glucuronic acid-based trigger elements are used in the conjugates of Structure (II). In some embodiments, a trigger element comprises a β-galactoside-based linker. β-Galactoside is present abundantly within lysosomes, while the enzyme activity outside cells is low.

A trigger element may include one or more peptides. In some embodiments, a peptide can be selected to contain natural amino acids, unnatural amino acids, or any combination thereof. In some embodiments, a peptide can be a tripeptide or a dipeptide. In particular embodiments, a dipeptide comprises L-amino acids, such as Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Trigger elements and immolative groups are known (WO 2022/076905) which is hereby incorporated by reference in their entirety. One immolative unit can be a bifunctional para-aminobenzyl alcohol group, which can link to a trigger element through an amino group, forming an amide bond, while an amine containing isoindolinone-glutarimide moiety can be attached through carbamate functionalities to the benzylic hydroxyl group of the para-aminobenzyl alcohol (to give ap-amidobenzylcarbamate. The resulting pro-compound can be activated upon protease-mediated cleavage, leading to a 1,6-elimination reaction releasing the unmodified isoindolinone-glutarimide moiety and remnants of the antibody-linker.

In some embodiments, an immolative unit (IM) comprises para-aminobenzyloxycarbonyl, an aminal, a hydrazine, a disulfide, an amide, an ester, a hydrazine, a phosphotriester, a diester, a β-glucuronide, a double bond, a triple bond, an ether bond, a ketone, a diol, a cyano, a nitro, a quaternary amine, or combinations thereof. In certain embodiments, an immolative unit comprises a paramethoxybenzyl, a dialkyldialkoxysilane, a diaryldialkoxysilane, an orthoester, an acetal, an optionally substituted β-thiopropionate, a ketal, a phosphoramidate, a hydrazone, a vinyl ether, an imine, an aconityl, a trityl, a polyketal, a bis-arylhydrazone, a diazobenzene, a vivinal diol, a pyrophosphate diester, or combinations thereof.

In certain embodiments, an immolative unit (IM) comprises one of the following structures:

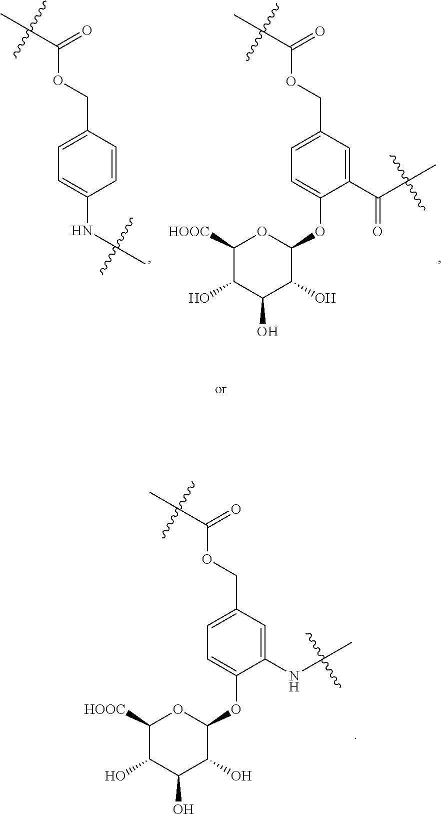 [SMILES: *Nc1ccc(COC(*)=O)cc1 ; *C(=O)OCc1ccc(O[C@@H]2O[C@H](C(=O)O)[C@@H](O)[C@H](O)[C@H]2O)c(C(*)=O)c1 ; *Nc1cc(COC(*)=O)ccc1O[C@@H]1O[C@H](C(=O)O)[C@@H](O)[C@H](O)[C@H]1O]

In some embodiments, an immolative unit comprises the following structure:

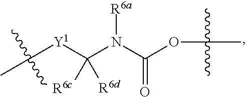

wherein:
R^{6a}, R^{6b}, R^{6c}, and R^{6d} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y² is —O—, —S—, or —NR^{6b}—;

In certain embodiments, an immolative unit comprises the following structure:

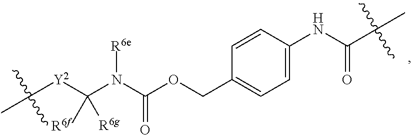

wherein:
R^{6e}, R^{6f}, R^{6g}, and R^{6h} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y² is —O—, —S—, or —NR^{6f}—;

In some embodiments, an immolative unit comprises the following structure:

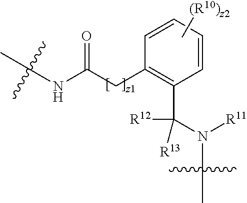

wherein:
each occurrence of R¹⁰ is independently alkyl, alkoxy, or halo;
R¹¹ is hydrogen, alkyl, or —(CH₂CH₂O)_{z3}—CH₃;
R¹² is hydrogen or alkyl;
R¹³ is hydrogen or alkyl;
z1 is 0 or 1;
z2 is 0, 1, 2, 3, or 4; and
z3 is 0, 1, 2, 3, 4, 5, 6, 7, 8, 9, or 10; or

In some embodiments, an immolative unit comprises one of the following structures:

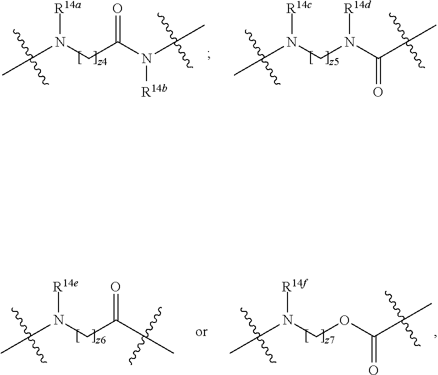

wherein:
R^{14a}, R^{14b}, R^{14c}, R^{14d}, R^{14e}, and R^{14f} are each independently hydrogen, alkyl, hydroxyalkyl, or alkoxyalkyl;
z4, z5, z6, and z7 are each independently 1, 2, 3, 4, 5, or 6;

In some embodiments, an immolative unit comprises one of the following structures:

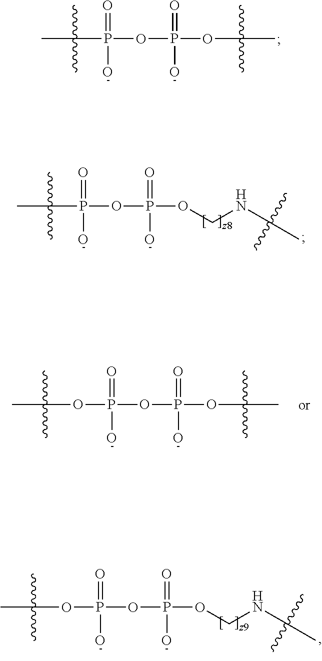

wherein:
z8 and z9 are each independently 1, 2, 3, 4, 5, or 6; or

In certain embodiments, an immolative unit comprises one of the following structures:

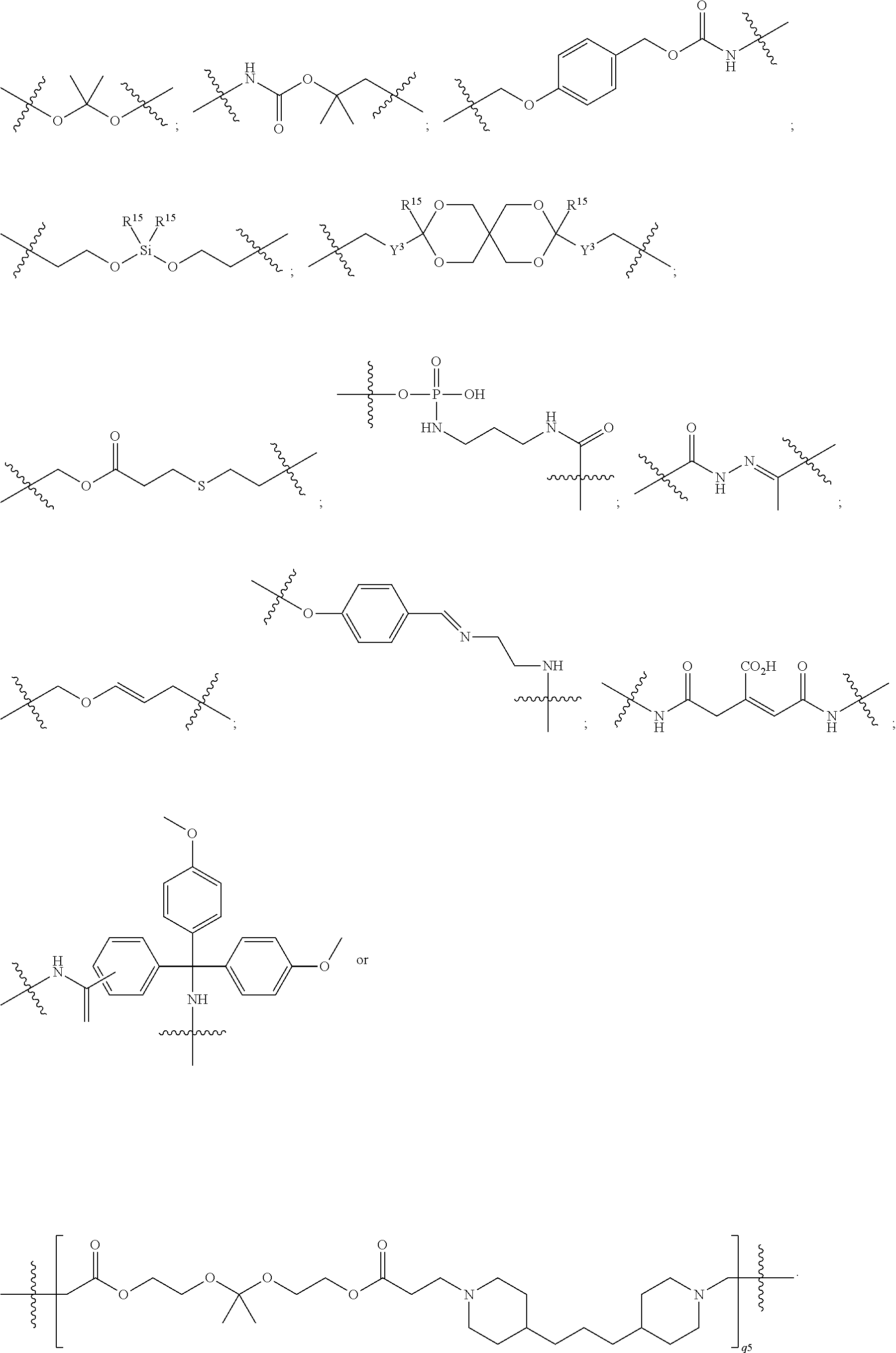

wherein:
each occurrence of R¹⁵ is independently H, methyl, ethyl, isopropyl, tert-butyl, or phenyl;
Y³ is O or CH₂; and
q5 is an integer ranging from 1-5.

In some embodiments, an immolative unit has a molecular weight greater than 150 g/mole, greater than 200 g/mole, greater than 300 g/mole, greater than 400 g/mole, greater than 500 g/mole, greater than 600 g/mole, greater than 700 g/mole, greater than 800 g/mole, greater than 900 g/mole, or greater than 1000 g/mole. In some embodiments, an immolative unit has a molecular weight less than 150 g/mole, less than 200 g/mole, less than 300 g/mole, less than 400 g/mole, less than 500 g/mole, less than 600 g/mole, less than 700 g/mole, less than 800 g/mole, less than 900 g/mole, or less than 1000 g/mole.

In some embodiments, an immolative unit and a trigger element together have the following structure:

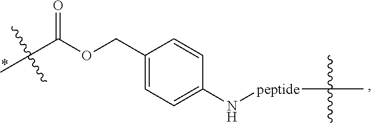

wherein a trigger element is denoted with “peptide” and comprises from one to ten amino acids, and, [symbol] represents the point of attachment to a isoindolinone-glutarimide moiety. In some embodiments, the peptide comprises Val-Cit or Val-Ala. Heterocyclic variants (e.g., pyridinyl, pyrimidinyl, etc.) of this immolative unit may also be used.

In some embodiments, an immolative unit contains a phenol group that is covalently bound to the remainder of the molecule through the phenolic oxygen. One such immolative unit relies on a methodology in which a diamino-ethane “Space Link” is used in conjunction with traditional “PABO”-based immolative unit to deliver phenols.

In some embodiments, a trigger element can include non-cleavable portions or segments. Polyethylene glycol (PEG) and related polymers can be included with cleavable groups such as a disulfide, a hydrazone or a dipeptide to form an immolative group and/or trigger element.

Other degradable linkages that can be included in immolative units can include esters. Esters can be formed by the reaction of PEG carboxylic acids or activated PEG carboxylic acids with alcohol groups on a isoindolinone-glutarimide moiety such ester groups can hydrolyze under physiological conditions to release a isoindolinone-glutarimide moiety. Other hydrolytically degradable linkages can include carbonate linkages, imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including at the end of a polymer, and a 5′ hydroxyl group of an oligonucleotide.

In some embodiments, a trigger element, immolative unit (IM), and isoindolinone-glutarimide moiety (IG) together have the following structure:

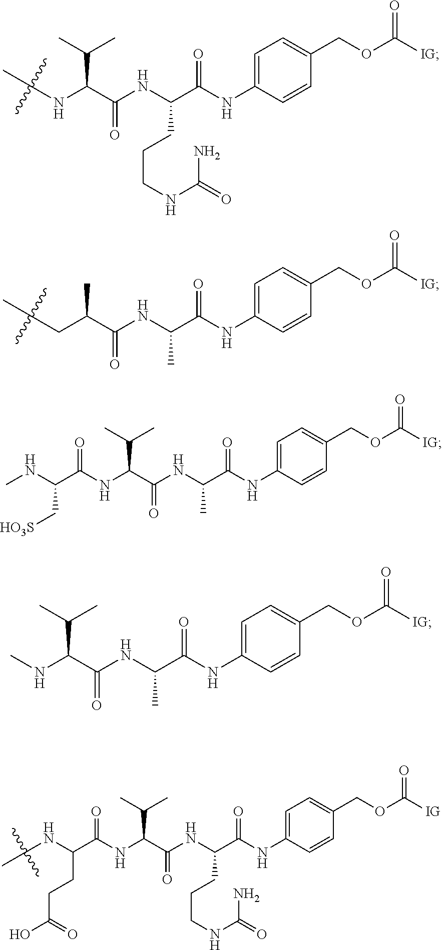

wherein [symbol] indicates an attachment site to the remainder of the molecule (i.e., a compound of Structure (I) or conjugate of Structure (II)) and an isoindolinone-glutarimide moiety (IG). Amino acids of embodiments above may be replaced or used in addition to other amino acids, in some embodiments, a trigger element is Asn-Cit, Arg-Cit, Val-Glu, Ser-Cit, Lys-Cit, Asp-Cit, Phe-Lys, Glu-Val-Cit, Glu-Val-Cit, Glu-Glu-Val-Cit, or Glu-Glu-Glu-Val-Cit, and an immolative unit is PABC.

In some embodiments, the phenyl portion of the PABC is substituted with one or more substituents. In some embodiments, the substituents have one of the following structures:

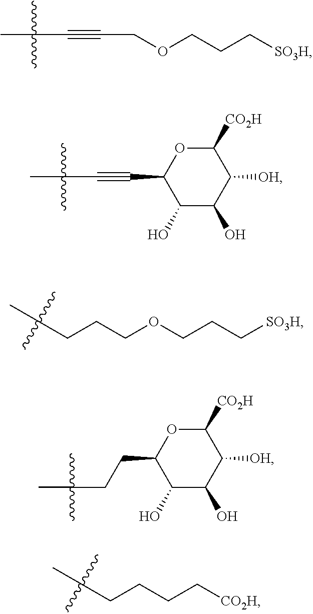 [SMILES: *C#CCOCCCS(=O)(=O)O ; *C#C[C@H]1O[C@@H](C(=O)O)[C@H](O)[C@@H](O)[C@@H]1O ; *CCCOCCCS(=O)(=O)O ; *CC[C@H]1O[C@@H](C(=O)O)[C@H](O)[C@@H](O)[C@@H]1O ; *CCCCC(=O)O]

In some embodiments, an immolative group comprises one of the following structures:

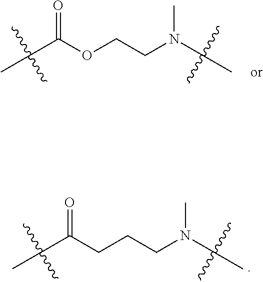

In some embodiments, a trigger element, an immolative unit (IM), and isoindolinone-glutarimide moiety (IG) together have one of the following structures:

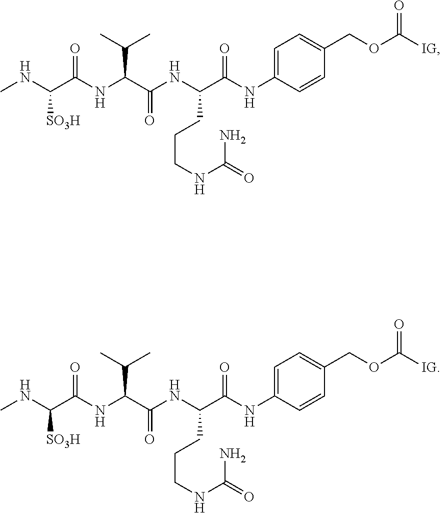

In some embodiments, an immolative unit (IM) has a structure selected from the following:

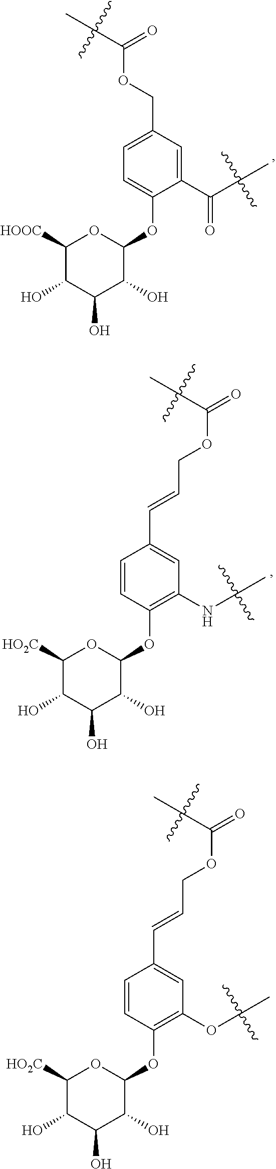 [SMILES: *C(=O)OCc1ccc(O[C@@H]2O[C@H](C(=O)O)[C@@H](O)[C@H](O)[C@H]2O)c(C(*)=O)c1 ; *Nc1cc(/C=C/COC(*)=O)ccc1O[C@@H]1O[C@H](C(=O)O)[C@@H](O)[C@H](O)[C@H]1O ; *Oc1cc(/C=C/COC(*)=O)ccc1O[C@@H]1O[C@H](C(=O)O)[C@@H](O)[C@H](O)[C@H]1O]

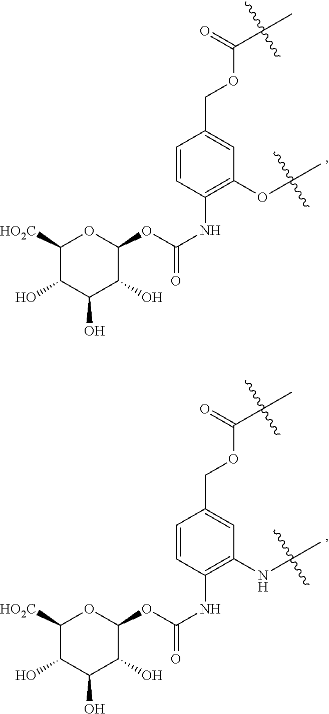 [SMILES: *Oc1cc(COC(*)=O)ccc1NC(=O)O[C@@H]1O[C@H](C(=O)O)[C@@H](O)[C@H](O)[C@H]1O ; *Nc1cc(COC(*)=O)ccc1NC(=O)O[C@@H]1O[C@H](C(=O)O)[C@@H](O)[C@H](O)[C@H]1O]

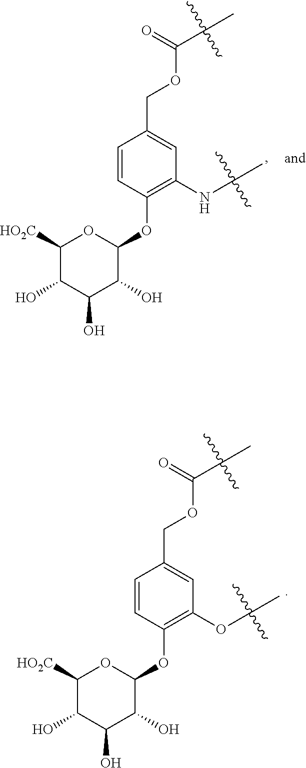 [SMILES: *Nc1cc(COC(*)=O)ccc1O[C@@H]1O[C@H](C(=O)O)[C@@H](O)[C@H](O)[C@H]1O ; *Oc1cc(COC(*)=O)ccc1O[C@@H]1O[C@H](C(=O)O)[C@@H](O)[C@H](O)[C@H]1O]

The structures above show a substitution pattern of 1, 3, 4 on the phenyl ring of an immolative unit (IM). In some embodiments, a substitution pattern may be 1, 2, 4 (i.e., 1 being a linkage to an IG, 2 being a linkage to the remainder of the molecule and 4 being a linkage to the carbohydrate) or 1, 3, 5 (i.e., 1 being a linkage to an IG, 3 being a linkage to the remainder of the molecule and 4 being a linkage to the carbohydrate).

Although cleavable linkers (e.g., linkers with trigger elements or immolative unit) can provide certain advantages, linkers need not be cleavable. For non-cleavable linkers, an IG release may not depend on the differential properties between the plasma and some cytoplasmic compartments. The release of an IG can occur after internalization of the conjugate of Structure (II) via antigen-mediated endocytosis and delivery to lysosomal compartment, where the targeting moiety (or binding fragment thereof) can be degraded to the level of amino acids through intracellular proteolytic degradation. This process can release a isoindolinone-glutarimide moiety or isoindolinone-glutarimide moiety derivative. A isoindolinone-glutarimide moiety or isoindolinone-glutarimide moiety derivative can be more hydrophilic and less membrane permeable, which can lead to less bystander effects and less non-specific toxicities compared to conjugates with a cleavable linker. Conjugates with non-cleavable linkers can have greater stability in circulation than conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units. In some embodiments, -L¹-R¹ or L²-R² comprises a linker that is non-cleavable in vivo.

In some embodiments, a trigger element and an immolative unit (IM) together comprise one of the following structures:

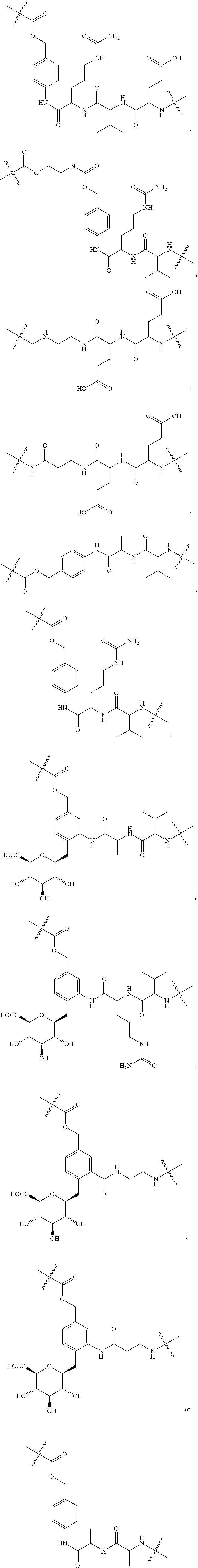

In some embodiments, a heteroalkylene element comprises polyethylene glycol or polypropylene glycol. In some embodiments, a heteroalkylene element comprises one of the following structures:

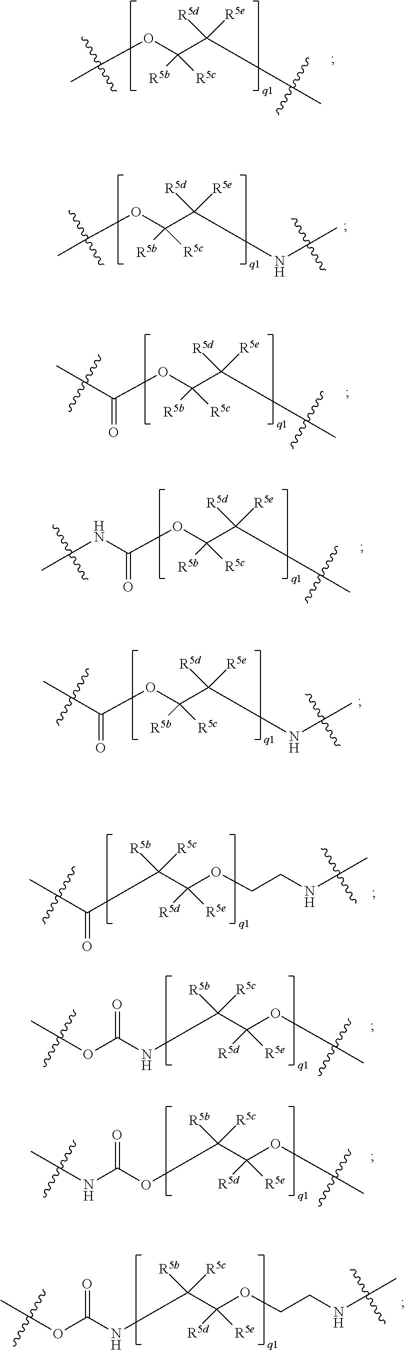

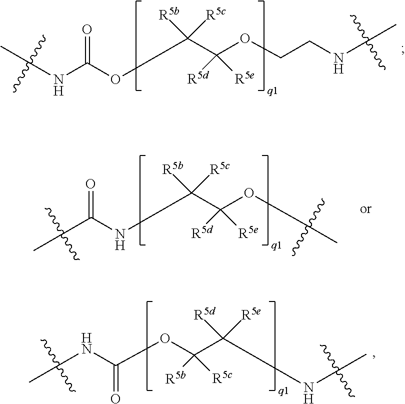

wherein:
each occurrence of R^{5b}, R^{5c}, R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H, each occurrence of q1 is, independently an integer from 1-24.

In some embodiments, R^{5b}, R^{5c}, R^{5d}, and R^{5e} are all hydrogen.

In some embodiments, a polar cap comprises one or more charged amino acid, one or more polyol, or combinations thereof. In certain embodiments, a polar cap comprises a diol, a triol, a tetraol, or combinations thereof. In some embodiments, a polar cap comprises glycerol, trimethylolpropane, pentaerythritol, maltitol, sorbitol, xylitol, erythritol, isomalt, or combinations thereof. In certain embodiments, a polar cap comprises one or more natural amino acids. In some embodiments, a polar cap comprises one or more non-natural amino acids. In some embodiments, a polar cap comprises one or more non-natural amino acids and one or more natural amino acids. In certain embodiments, a polar cap comprises serine, threonine, cysteine, proline, asparagine, glutamine, lysine, arginine, histidine, aspartate, glutamate, 4-hydroxyproline, 5-hydroxylysine, homoserine, homocysteine, ornithine, beta-alanine, statine, or gamma aminobutyric acid. In certain embodiments, a polar cap comprises aspartic acid, serine, glutamic acid, serine-beta-glucose, or combinations thereof.

In some embodiments, a polar cap comprises one of the following structures:

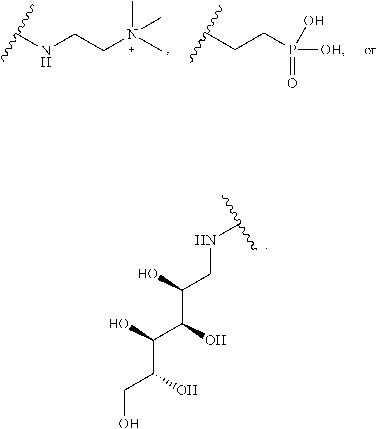

In more embodiments, a polar cap has one of the following structures, including combinations thereof:

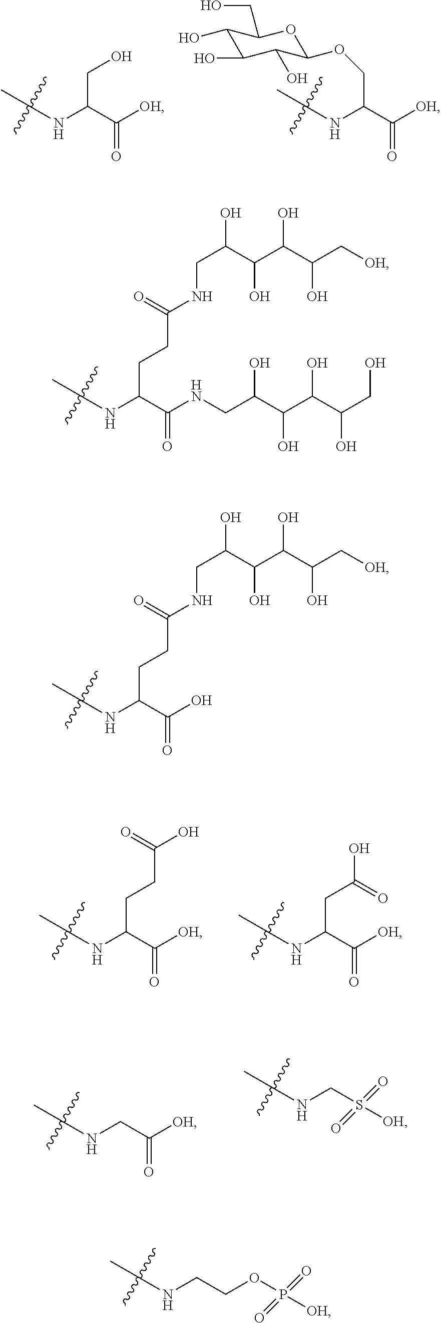

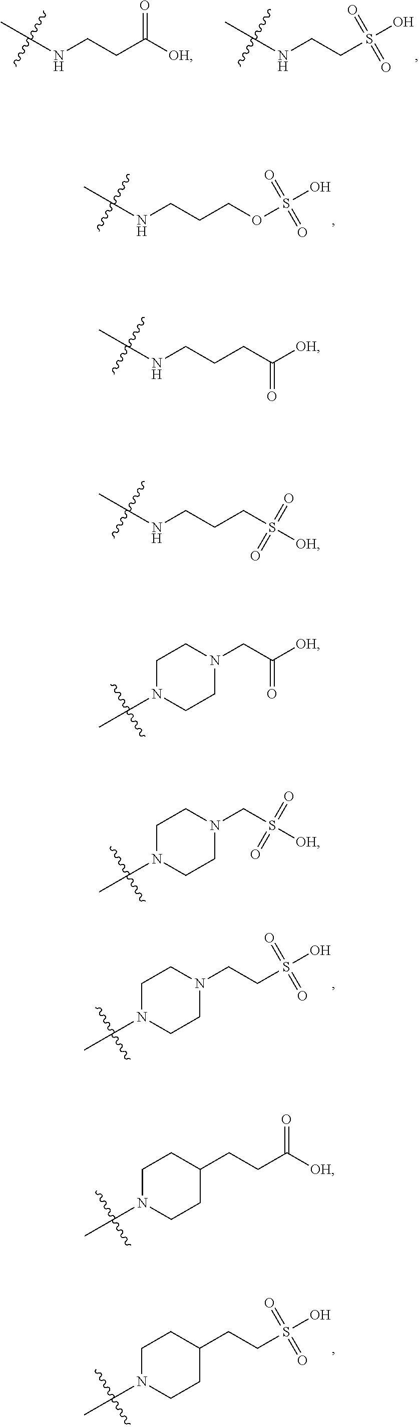

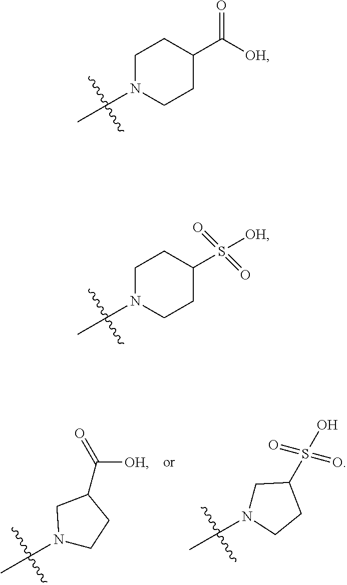 [SMILES: *N1CCC(C(=O)O)CC1 ; *N1CCC(S(=O)(=O)O)CC1 ; *N1CCC(C(=O)O)C1 ; *N1CCC(S(=O)(=O)O)C1]

In more embodiments, L¹, L², or L³ comprise a linker selected from the group alkylene, alkylene-L^{a}-, alkenylene, alkenylene-L^{a}-, alkynylene, alkynylene-L^{a}-, -L^{a}-, -L^{a}-alkylene-L^{a}-, -L^{a}-alkenylene-L^{a}-, -L^{a}-alkynylene-L^{a}-, and combinations thereof, wherein each alkylene, alkenylene, and alkynylene is optionally substituted and each occurrence of L^{a} is independently selected from —O—, —S—, —N(R⁷)—, —C(O)—, —C(S)—, —C(O)O—, —OC(O)—, —OC(O)O—, —C(O)N(R⁷)—, —N(R⁷)C(O)—, —C(O)N(R⁷)C(O)—, —C(O)N(R⁷)C(O)N(R⁷), —N(R⁷)C(O)N(R⁷)—, —N(R⁷)C(O)O—, —OC(O)N(R⁷)—, —C(NR⁷)—, —N(R⁷)C(NR⁷)—, —C(NR⁷)N(R⁷)—, —N(R⁷)C(NR⁷)N(R⁷)—, —S(O)₂—, —OS(O)—, —S(O)O—, —S(O), —OS(O)₂—, —S(O)₂O, —N(R⁷)S(O)₂—, —S(O)₂N(R⁷)—, —N(R⁷)S(O)—, —S(O)N(R⁷)—, —N(R⁷)S(O)₂N(R⁷)—, and —N(R⁷)S(O)N(R⁷)— and R⁷ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ cycloalkyl, and 3- to 12-membered heterocycle, each of which is optionally substituted with one or more substituents independently selected from halo, hydroxyl, cyano, nitro, amino, oxo, thioxo, —C(O)OCH₂C₆H₅, —NHC(O)OCH₂C₆H₅, C₁₋₁₀ alkyl, C₁₋₁₀ haloalkyl, C₁₋₁₀ alkoxy, C₂₋₁₀ alkenyl, C₂₋₁₀ alkynyl, C₃₋₁₂ cycloalkyl, and 3- to 12-membered heterocyclyl.

In some embodiments, each L¹, L², or L³ is optionally substituted with one or more substituents selected from alkyl, alkenyl, alkynyl, halo, hydroxyl, cyano, —OR⁸, —SR⁸, amino, aminyl, amido, cycloalkyl, aryl, heterocyclyl, heteroaryl, cycloclkylalkyl, arylalkyl, heterocyclylalkyl, heteroarylalkyl, —C(O)R⁸, —C(O)N(R⁸)₂, —N(R⁸)C(O)R⁸, —C(O)OR⁸, —OC(O)R⁸, —S(O)R⁸, —S(O)₂R⁸, —P(O)(OR⁸)₂, —OP(O)(OR⁸)₂, nitro, oxo, thioxo, ═N(R⁸), or cyano, and R⁸ is hydrogen, alkyl, alkenyl, alkynyl, cycloalkyl, aryl, heterocyclyl, heteroaryl, cycloclkylalkyl, arylalkyl, heterocyclylalkyl, or heteroarylalkyl.

In some embodiments, L¹, L², or L³ are independently selected from the following structures:

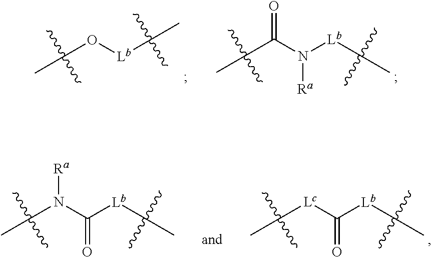

wherein:
R^{a} is hydrogen or alkyl;
each occurrence of L^{b} is independently a direct bond, an optionally substituted alkylene linker, an optionally substituted heteroalkylene linker, a heteroatomic linker, or a combination thereof; and each occurrence of L^{c} is independently an optionally substituted alkylene linker and provided that at least one of L¹, L², or L³ has the following structure:

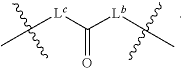

In some embodiments, L¹ and L² are independently selected from the following structures:

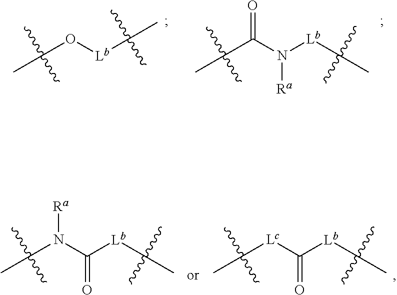

wherein:
R^{a} is hydrogen or alkyl;
each occurrence of L^{b} is independently a direct bond, an optionally substituted alkylene linker, an optionally substituted heteroalkylene linker, a heteroatomic linker, or a combination thereof;
each occurrence of L^{c} is independently an optionally substituted alkylene linker; provided that at least one of L¹ or L² has the following structure:

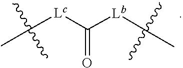

In more embodiments, L² has the following structure:

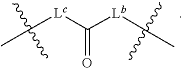

In some embodiments, L^{c} is unsubstituted. In some embodiments, L^{c} is a C₁-C₆ alkylene. In some more embodiments, L^{c} is a C₂-C₄ alkylene. In some embodiments, L^{c} is a straight C₁-C₆ alkylene. In more embodiments, L^{c} is a straight, unsubstituted C₁-C₆ alkylene. In more embodiments, L^{c} is a straight, unsubstituted C₂-C₄ alkylene.

In some embodiments, the isoindolinone-glutarimide linker compound has one of the following structures (Ia-1), (Ia-2), (Ia-3), (Ia-4), (Ia-5), or (Ia-6):

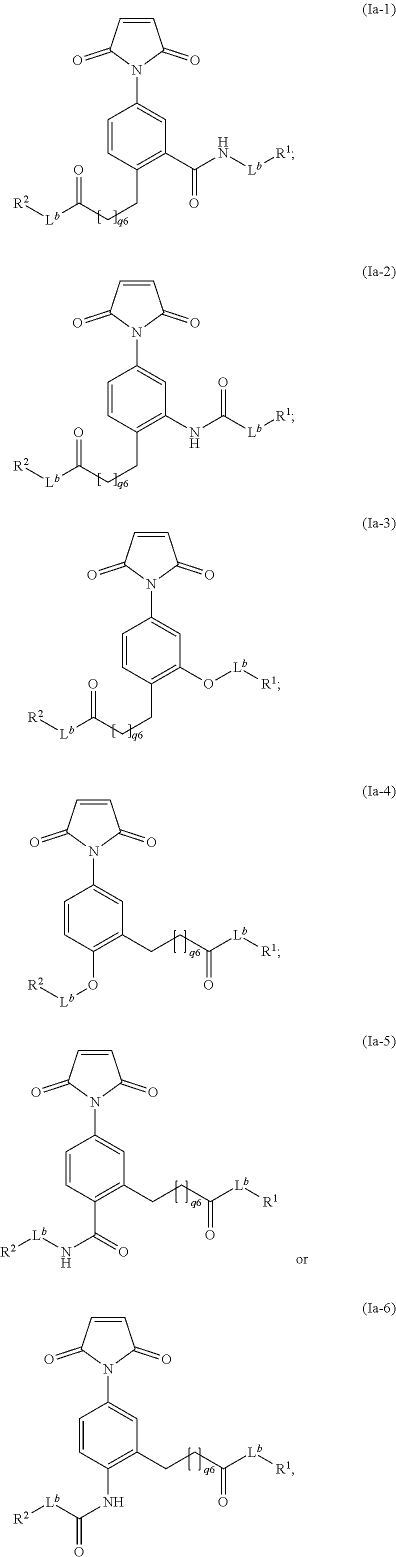

wherein:
each occurrence of L^{b} is independently a direct bond, an optionally substituted alkylene linker, an optionally substituted heteroalkylene linker, a heteroatomic linker, or combinations thereof; and
q6 is 0, 1, or 2.

In some embodiments, q6 is 1 and L^{b} is gly-gly.

In certain embodiments, the isoindolinone-glutarimide linker compound has one of the following Structures (Ic-1), (Ic-2), (Ic-3), or (Ic-4):

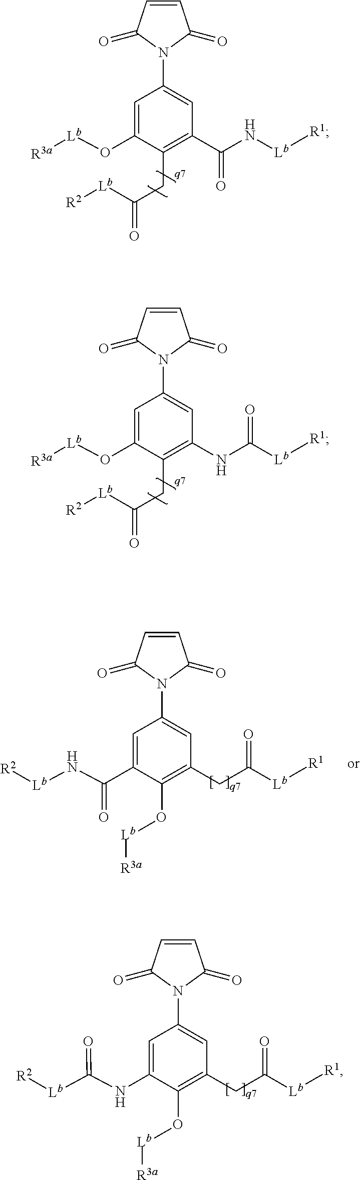

wherein:
each occurrence of L^{b} is independently a direct bond, an optionally substituted alkylene linker, an optionally substituted heteroalkylene linker, a heteroatomic linker, or combinations thereof; and
q7 is 1, 2, or 3.

In some embodiments, q7 is 2.

In some embodiments, the isoindolinone-glutarimide linker compound has the following Structure (Id), (Ie), (If), or (Ig):

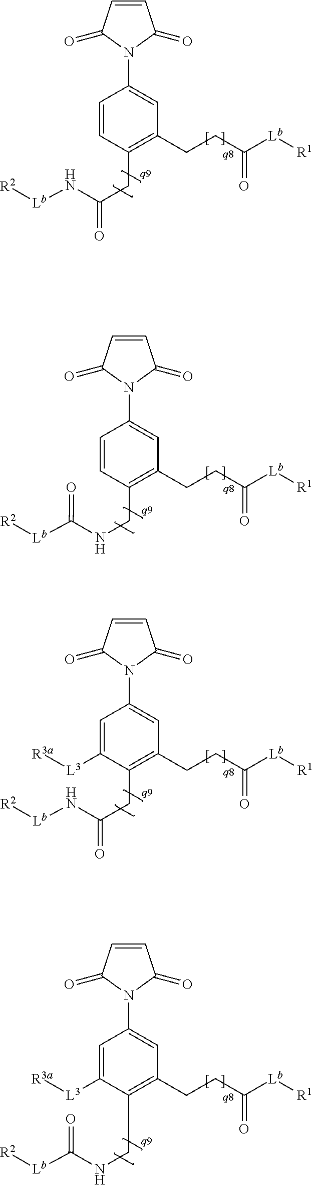

wherein:
each occurrence of L^{b} is independently a direct bond, an optionally substituted alkylene linker, an optionally substituted heteroalkylene linker, a heteroatomic linker, or combinations thereof;
q8 is 0, 1, or 2; and
q9 is, 1, or 2.

In some embodiments, q9 is 0 and q8 is 1.

In some embodiments, the isoindolinone-glutarimide linker compound has one of the following Structures (Ih) or (Ii):

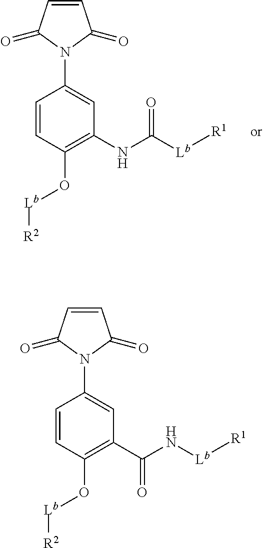

wherein:
each occurrence of L^{b} is independently a direct bond, an optionally substituted alkylene linker, an optionally substituted heteroalkylene linker, a heteroatomic linker, or combinations thereof.

In some embodiments, L^{b} is a direct bond, an optionally substituted alkylene linker or an optionally substituted heteroalkylene linker.

In some embodiments, L^{b} is a direct bond or has one of the following structures:

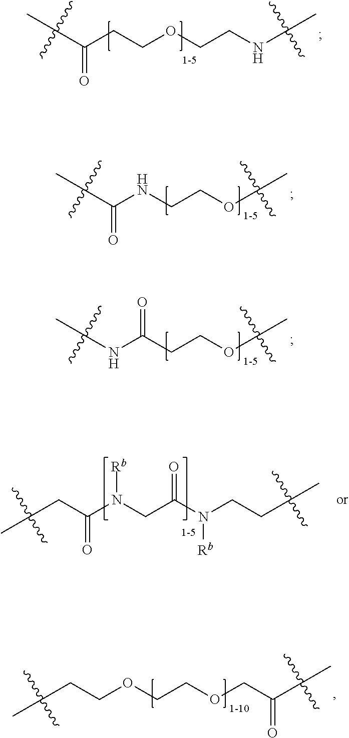

wherein:
each occurrence of R^{b} is independently hydrogen, alkyl, hydroxyalkyl, or alkoxyalkyl.

In some embodiments, each occurrence of R^{b} is —CH₃.

In some embodiments, L¹ or L² has the following structure:

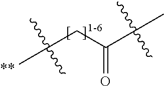

wherein:
** shows a bond to X¹, X², X³, X⁴ or X⁵.

In certain embodiments, X² is C-L¹-R¹ and X³ is C-L²-R². In some embodiments, X³ is C-L¹-R¹ and X² is C-L²-R². In some embodiments, X¹, X⁴, and X⁵ are all CR³. In some embodiments, X¹, X⁴, and X⁵ are all CH.

### Isoindolinone-Glutarimide Linker Compounds

The conjugates of the invention are prepared by conjugation of an antibody with an isoindolinone-glutarimide linker compound, IG-L.

An isoindolinone-glutarimide linker compound is selected from Formulae IIa and IIb:

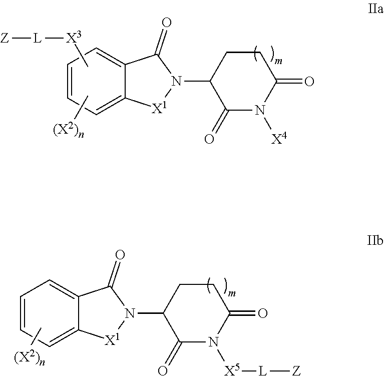

wherein:
m is 1 or 2;
X¹ is selected from the group consisting of CH₂, C(═O) and N═N;
X² is independently selected from the group consisting of F, Cl, Br, I, —CN, C₁-C₁₂ alkyl, C₂-C₁₂ alkenyl, C₂-C₁₂ alkynyl, C₁-C₁₂ heteroalkyl, —(C₁-C₁₂ heteroalkyldiyl)-(C₆-C₂₀ aryldiyl), —(C₁-C₁₂ heteroalkyldiyl)-(C₆-C₂₀ aryldiyl)-(C₁-C₁₂ heteroalkyl), —(C₁-C₁₂ heteroalkyldiyl)-(C₆-C₂₀ aryl), —(C₁-C₁₂ heteroalkyldiyl)-(C₆-C₂₀ aryldiyl)-(C₁-C₁₂ heteroalkyl), (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, —OR^{a}, —OC(═O)R^{a}, —SR^{a}, —S(O)R^{a}—S(O)₂R^{a}—S(O)₂NR^{a}, and —S(O)₃H;
R^{a} is independently selected from H, C₁-C₆ alkyl, phenyl, and benzyl, wherein phenyl and benzyl are optionally substituted with one or more groups independently selected from the group consisting of F, Cl, CN, C₁-C₁₂ alkyl, C₂-C₁₂ alkenyl, and C₂-C₁₂ alkynyl;
R^{b} is independently selected from H, OH, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
n is 0, 1, 2, 3, or 4,
X³ is selected from the group consisting of a bond, O, C₁-C₁₂ alkyldiyl, C₂-C₁₂ alkenyldiyl, C₂-C₁₂ alkynyldiyl, C₁-C₁₂ heteroalkyldiyl, —(C₁-C₆ alkyldiyl)-(C₆-C₂₀ aryldiyl)-, —(C₁-C₆ alkyldiyl)NR^{a}—, —(C₁-C₆ alkyldiyl)O—, —(C₁-C₆ alkyldiyl)-(C₃-C₂₀ carbocyclyldiyl)-, —(C₁-C₆ alkyldiyl)-(C₂-C₂₀ heterocyclyldiyl)-, —(C₁-C₆ alkyldiyl)-(C₁-C₂₀ heteroaryldiyl)-, —(C₁-C₁₂ heteroalkyldiyl)-(C₆-C₂₀ aryldiyl)-, —(C₁-C₁₂ heteroalkyldiyl)-(C₆-C₂₀ aryldiyl)-(C₁-C₁₂ heteroalkyldiyl)-, —(C₁-C₁₂ heteroalkyldiyl)-(C₆-C₂₀ aryldiyl)-(C₁-C₁₂ heteroalkyldiyl)-(C₆-C₂₀ aryldiyl)-(C₁-C₁₂ heteroalkyldiyl)-, —(C₁-C₁₂ heteroalkyldiyl)-(C₆-C₂₀ aryl)-, C₆-C₂₀ aryldiyl, C₃-C₂₀ carbocyclyldiyl, C₂-C₂₀ heterocyclyldiyl, and C₁-C₂₀ heteroaryldiyl;
X⁴ is selected from the group consisting of H, C₁-C₁₂ heteroalkyl, —(C₁-C₁₂ heteroalkyldiyl)-(C₆-C₂₀ aryl), —(C₁-C₁₂ heteroalkyldiyl)-(C₆-C₂₀ aryldiyl)-(C₁-C₁₂ heteroalkyl);
X⁵ is selected from the group consisting of C₁-C₁₂ alkyldiyl, C₂-C₁₂ alkenyldiyl, C₂-C₁₂ alkynyldiyl, C₁-C₁₂ heteroalkyldiyl, —(C₁-C₆ alkyldiyl)-(C₆-C₂₀ aryldiyl), —(C₁-C₆ alkyldiyl)-(C₆-C₂₀ aryldiyl)-O—(C₂-C₂₀ heterocyclyl), —(C₁-C₆ alkyldiyl)NR^{a}—, —(C₁-C₆ alkyldiyl)O—, —(C₁-C₆ alkyldiyl)-(C₃-C₂₀ carbocyclyldiyl), —C₁-C₆ alkyldiyl)-(C₂-C₂₀ heterocyclyldiyl), —(C₁-C₆ alkyldiyl)-(C₁-C₂₀ heteroaryldiyl), —(C₁-C₁₂ heteroalkyldiyl)-(C₆-C₂₀ aryldiyl), —(C₁-C₁₂ heteroalkyldiyl)-(C₆-C₂₀ aryldiyl)-(C₁-C₁₂ heteroalkyldiyl), C₆-C₂₀ aryldiyl, C₃-C₂₀ carbocyclyldiyl, C₂-C₂₀ heterocyclyldiyl, and C₁-C₂₀ heteroaryldiyl;
L is the antibody linker; and
Z is:

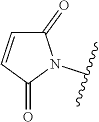 [SMILES: *N1C(=O)C=CC1=O]

where the wavy line is the attachment to L;
wherein each alkyl, alkyldiyl, alkenyl, alkenyldiyl, alkynyl, alkynyldiyl, heteroalkyl, heteroalkyldiyl, aryl, aryldiyl, carbocyclyl, carbocyclyldiyl, heterocyclyl, heterocyclyldiyl, heteroaryl, and heteroaryldiyl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of the isoindolinone-glutarimide linker compound includes wherein L has the formula:

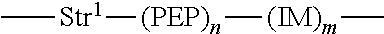

wherein:
Str¹ is a stretcher unit covalently attached to Z;
PEP is a protease-cleavable, peptide unit covalently attached to Str¹ and IM;
IM is an immolative unit covalently attached the isoindolinone-glutarimide moiety;
n is 0 or 1; and m is 0 or 1.

An exemplary embodiment of the isoindolinone-glutarimide linker compound includes wherein PEP-IM is selected from the structures:

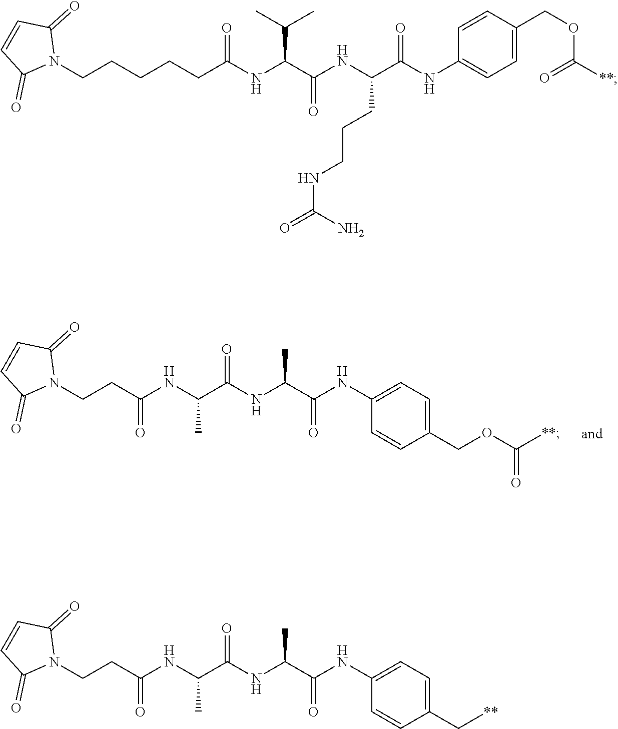

wherein ** indicates the point of attachment to the isoindolinone-glutarimide moiety.

An exemplary embodiment of the isoindolinone-glutarimide linker compound includes wherein Z-Str¹ has the structure:

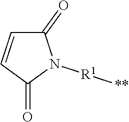

wherein:
** indicates the point of attachment to PEP or the isoindolinone-glutarimide moiety;
R¹ is selected from the group consisting of C₁-C₁₂ alkyldiyl, C₁-C₁₂ alkyldiyl-C(═O), C₁-C₁₂ alkyldiyl-NH, (CH₂CH₂O)ᵣ, (CH₂CH₂O)ᵣ—C(═O), (CH₂CH₂O)ᵣ—CH₂, C₆-C₂₀ aryldiyl, (C₆-C₂₀ aryldiyl)-(C₁-C₁₂ alkyldiyl), and (C₆-C₂₀ aryldiyl)-(C₁-C₁₂ heteroalkyldiyl);
r is an integer ranging from 1 to 10, and
alkyldiyl, heteroalkyldiyl, and aryldiyl are independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, —S(O)₃H, and a solubilizing unit.

An exemplary embodiment of the isoindolinone-glutarimide linker compound includes wherein R¹ is selected from —(CH₂)₅—, and —CH₂CH₂—.

An exemplary embodiment of the isoindolinone-glutarimide linker compound includes wherein R¹ is selected from C₆-C₂₀ aryldiyl, (C₆-C₂₀ aryldiyl)-(C₁-C₁₂ alkyldiyl), and (C₆-C₂₀ aryldiyl)-(C₁-C₁₂ heteroalkyldiyl).

An exemplary embodiment of the isoindolinone-glutarimide linker compound includes wherein Z-Str¹ has the structure:

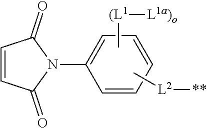

wherein:
L¹ is selected from a bond, C₁-C₁₂ alkyldiyl, and C₁-C₄₀ heteroalkyldiyl;
L¹a is selected from an amino acid, amino, hydroxyl, halide, hydrogen, carboxylic acid, glycerol, or a sugar such as pentaerythritol, maltitol, sorbitol, xylitol, erythritol, isomalt, or combinations thereof;
o is 0, 1, or 2;
L² is selected from a bond, C₁-C₁₂ alkyldiyl, and C₁-C₄₀ heteroalkyldiyl; and
** indicates the point of attachment to PEP or to the isoindolinone-glutarimide moiety.

An exemplary embodiment of the isoindolinone-glutarimide linker compound includes wherein L¹ or L² comprise a solubilizing unit selected from polyglycine, polysarcosine, polyethyleneoxy (PEG), C₁-C₄₀ heteroalkyl, and a glycoside, or combinations thereof.

An exemplary embodiment of the isoindolinone-glutarimide linker compound includes wherein L¹ or L² is selected from (N(CH₃)CH₂C(═O))_{q}, (N(CH₃)CH₂CH₂C(═O))_{q}, N(CH₃)CH₂CH₂OCH₂CH₂C(═O))_{q}, (CH₂CH₂O)_{q}, (CH₂CH₂O)_{q}C(═O), and (CH₂CH₂O)_{q}—CH₂, where q is an integer from 2 to 20.

The invention includes all reasonable combinations, and permutations of the features, of the Formula II embodiments.

The linker units comprise functional groups and subunits which affect stability, permeability, solubility, and other pharmacokinetic, safety, and efficacy properties of the antibody conjugates. The linker unit includes a reactive functional group which reacts, i.e. conjugates, with a reactive functional group of the antibody. For example, a nucleophilic group such as a lysine side chain amino of the antibody reacts with an electrophilic reactive functional group of the isoindolinone-glutarimide-linker, IG-L compound to form the antibody conjugate. Also, for example, a cysteine thiol of the antibody reacts with a maleimide or bromoacetamide group of the IG-L compound to form the antibody conjugate.

Considerations for the design of the antibody conjugates of the invention include: (1) preventing the premature release of the isoindolinone-glutarimide (IG) moiety during in vivo circulation and (2) ensuring that a biologically active form of the IG moiety is released at the desired site of action at an adequate rate. The complex structure of the antibody conjugate together with its functional properties requires careful design and selection of every component of the molecule including antibody, conjugation site, linker structure, and the IG moiety. The linker determines the mechanism and rate of isoindolinone-glutarimide moiety release.

Generally, the linker unit (L) may be cleavable or non-cleavable. Cleavable linker units may include a peptide sequence which is a substrate for certain proteases such as Cathepsins which recognize and cleave the peptide linker unit, separating the isoindolinone-glutarimide moiety from the antibody (Caculitan N G, et al (2017) Cancer Res. 77(24):7027-7037).

Cleavable linker units may include labile functionality such as an acid-sensitive disulfide group (Kellogg, B A et al (2011) Bioconjugate Chem. 22, 717-727; Ricart, A. D. et al (2011) Clin. Cancer Res. 17, 6417-6427; Pillow, T., et al (2017) Chem. Sci. 8, 366-370; Zhang D, et al (2016) ACS Med Chem Lett. 7(11):988-993).

In some embodiments, the linker is non-cleavable under physiological conditions. As used herein, the term “physiological conditions” refers to a temperature range of 20-40 degrees Celsius, atmospheric pressure (i.e., 1 atm), a pH of about 6 to about 8, and the one or more physiological enzymes, proteases, acids, and bases. One advantage of a non-cleavable linker between the antibody and IG moiety in an antibody conjugate is minimizing premature isoindolinone-glutarimide moiety release and corresponding toxicity.

In some embodiments, the linker comprises a trivalent, branch point as part of an amino acid unit (e.g., lysine) wherein additional linker units are attached via the side chain amine of lysine or linked to other sites of an amino acid unit (U.S. Pat. No. 11,173,214). A similar motif could be utilized with a glutamic acid of an amino acid unit. An exemplary additional linker unit is a monovalent solubilizing unit such as one or more units of polyglycine, polysarcosine, polyethyleneoxy (PEG), C₁-C₄₀ heteroalkyl, and a glycoside, or combinations thereof. The solubilizing unit may bear a group at the terminus such as an amino acid, amino, hydroxyl, hydrogen, carboxylic acid, glycerol, or a sugar such as pentaerythritol, maltitol, sorbitol, xylitol, erythritol, isomalt, or combinations thereof.

In some embodiments, a solubilizing unit is a polysarcosine with one of the following structures:

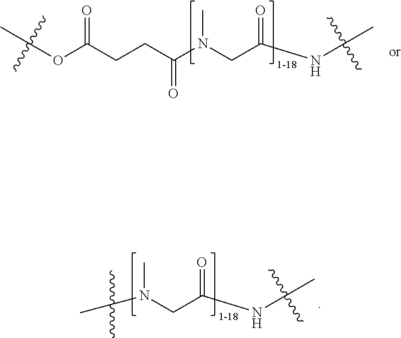

In some embodiments, an amino acid unit or peptide unit comprises one or more amino acids selected from the group consisting of glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, sarcosine, and beta-alanine.

In one embodiment, the invention includes an amino acid unit or a peptide linking unit, i.e. L or linker, between the antibody and the IG moiety, comprising a peptide comprising a linear sequence of specific amino acid residues which can be selectively cleaved by a protease such as a cathepsin, caspase, a tumor-associated elastase enzyme or an enzyme with protease-like or elastase-like activity. The peptide radical may be two to about twelve amino acids. Enzymatic cleavage of a bond within the peptide linker releases an active form of the IG moiety. This leads to an increase in the tissue specificity of the antibody conjugates and thus to an additional decrease of toxicity of the conjugates according to the invention in other tissue types. Release of an active IG moiety from an antibody conjugate can occur due to the action of lysosomal proteases such as cathepsin and plasmin which may be present at elevated levels in certain tumor tissues. The lysosomal enzyme can be, for example, cathepsin B, β-glucuronidase, or β-galactosidase.

A cleavable peptide of a peptide linker unit can be selected from tetrapeptides such as Gly-Phe-Leu-Gly, Ala-Leu-Ala-Leu, and Gly-Gly-Phe-Gly; tripeptides such as Glu-Val-Cit, and Ala-Ala-Ala; and dipeptides such as Val-Cit, Val-Ala, Ala-Ala, and Phe-Lys.

The linker provides sufficient stability of the antibody conjugate in biological media, e.g. culture medium or serum and, at the same time, the desired intracellular action within tumor tissue as a result of its specific enzymatic or hydrolytic cleavability with release of the IG moiety, i.e. “isoindolinone-glutarimide moiety”.

The enzymatic activity of a protease, cathepsin, or elastase can catalyze cleavage of a covalent bond of the antibody conjugate under physiological conditions. The enzymatic activity being the expression product of cells associated with tumor tissue. The enzymatic activity on the cleavage site of the targeting peptide converts the antibody conjugate to an active IG drug free of targeting antibody and linking group. The cleavage site may be specifically recognized by the enzyme. Cathepsin or elastase may catalyze the cleavage of a specific peptidic bond between the C-terminal amino acid residue of the specific peptide and the IG moiety of the antibody conjugate.

In one embodiment, the invention includes a linking unit, i.e. L or linker, between the antibody and the IG moiety, comprising a substrate for glucuronidase (Jeffrey S C, et al (2006) Bioconjug Chem. 17(3):831-40; U.S. Pat. Nos. 11,413,353; 11,173,214), or sulfatase (Bargh J D, et al (2020) Chem Sci. 11(9):2375-2380) cleavage. In particular, L includes a Glue unit and comprises a formula selected from:

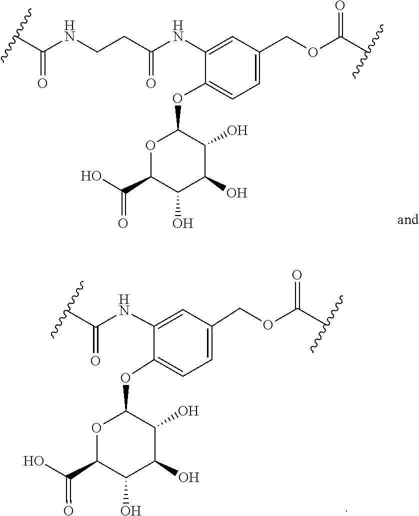 [SMILES: *C(=O)NCCC(=O)Nc1cc(COC(*)=O)ccc1O[C@@H]1O[C@H](C(=O)O)[C@@H](O)[C@H](O)[C@H]1O ; *C(=O)Nc1cc(COC(*)=O)ccc1O[C@@H]1O[C@H](C(=O)O)[C@@H](O)[C@H](O)[C@H]1O]

Specific cleavage of the antibody conjugate takes advantage of the presence of tumor infiltrating cells of the immune system and leukocyte-secreted enzymes, to promote the activation of an anticancer drug at the tumor site.

Reactive electrophilic reactive functional groups (Q in Formula II) suitable for the isoindolinone-glutarimide linker compound (IG-L) include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) and sulfotetrafluorophenyl (STP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include, but are not limited, to those described in Hermanson, Bioconjugate Techniques 2^{nd} Edition, Academic Press, 2008.

Some linkers such as those comprising peptide units and substrates for protease may be labile in the blood stream, thereby releasing unacceptable amounts of the drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release may have poor stability in the bloodstream. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. Aggregate formation may be correlated to the number of equivalents of drug moieties conjugated to the antibody. Under high drug loading, formed aggregates must be removed for therapeutic applications. As a result, drug loading-mediated aggregate formation decreases antibody conjugate yield and can render process scale-up difficult.

The invention includes all reasonable combinations, and permutations of the features, of the Formula IIa and IIb embodiments.

In some embodiments, the isoindolinone-glutarimide linker compound has one of the following structures:

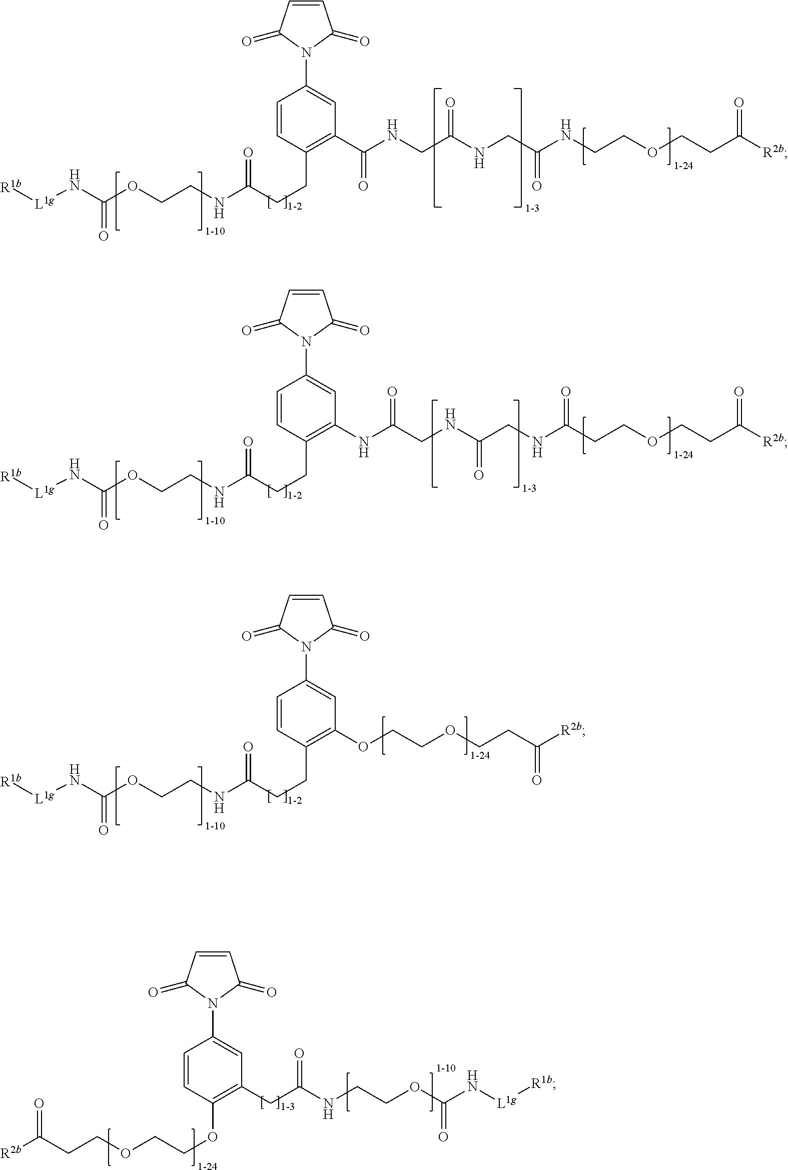

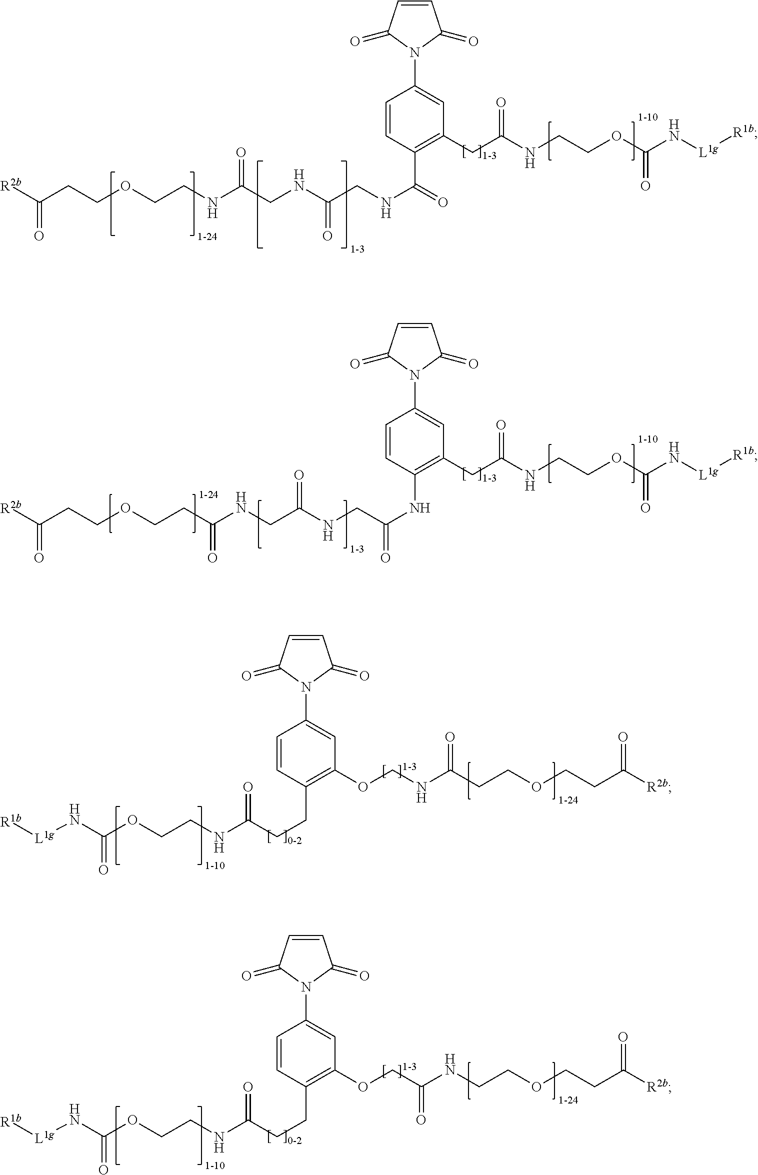

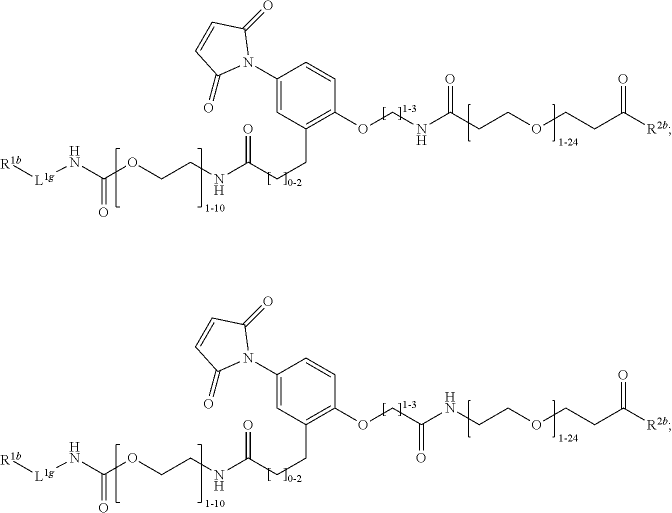

wherein:
R^{1b} is an isoindolinone-glutarimide moiety (IG);
R^{2b} has one of the following structures:

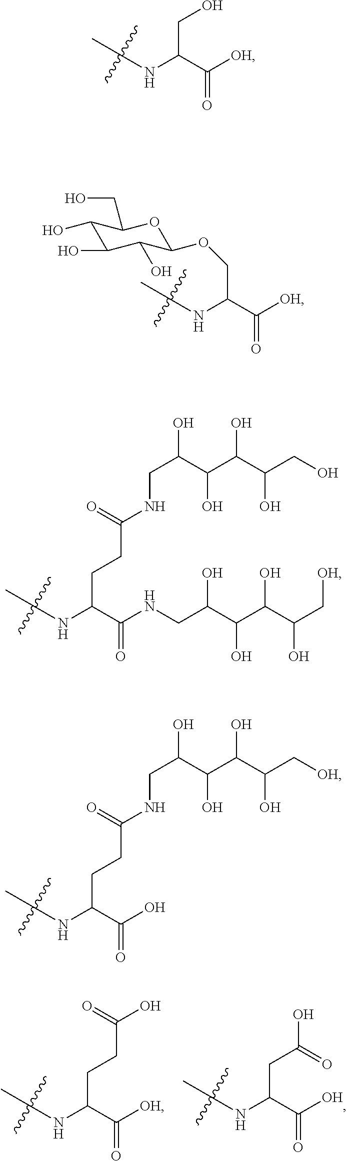 [SMILES: *NC(CO)C(=O)O ; *NC(COC1OC(CO)C(O)C(O)C1O)C(=O)O ; *NC(CCC(=O)NCC(O)C(O)C(O)C(O)CO)C(=O)NCC(O)C(O)C(O)C(O)CO ; *NC(CCC(=O)NCC(O)C(O)C(O)C(O)CO)C(=O)O ; *NC(CCC(=O)O)C(=O)O ; *NC(CC(=O)O)C(=O)O]

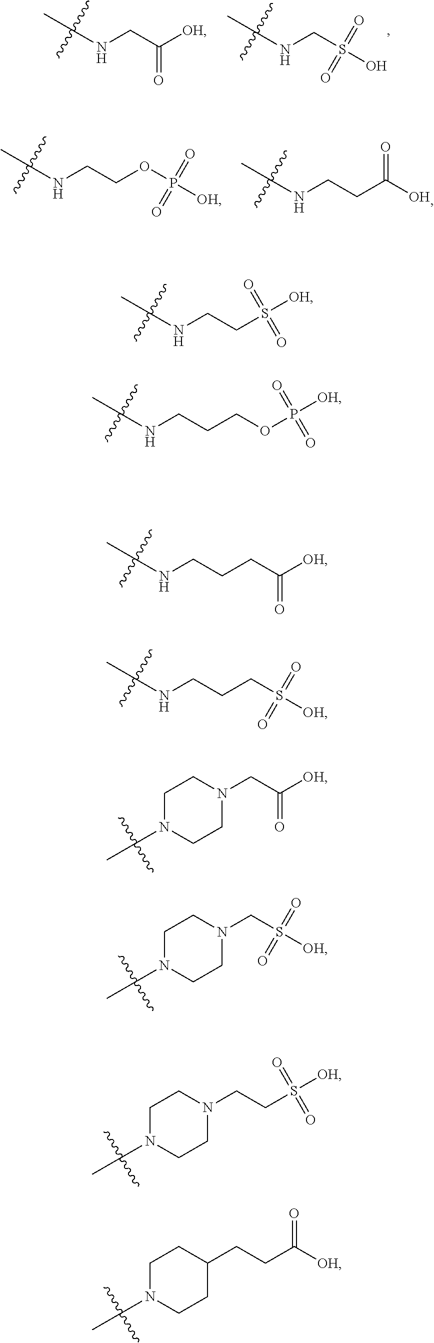

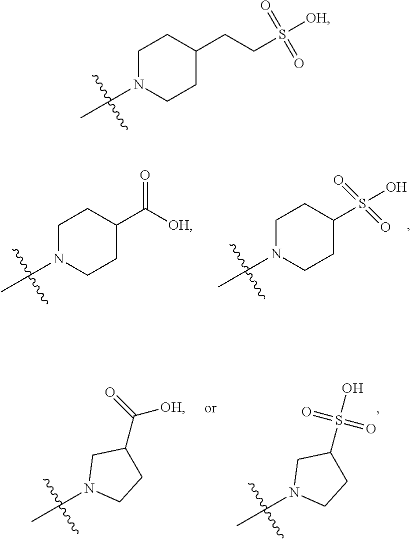 [SMILES: *N1CCC(CCS(=O)(=O)O)CC1 ; *N1CCC(C(=O)O)CC1 ; *N1CCC(S(=O)(=O)O)CC1 ; *N1CCC(C(=O)O)C1 ; *N1CCC(S(=O)(=O)O)C1]

L^{1g} has one of the following structures:

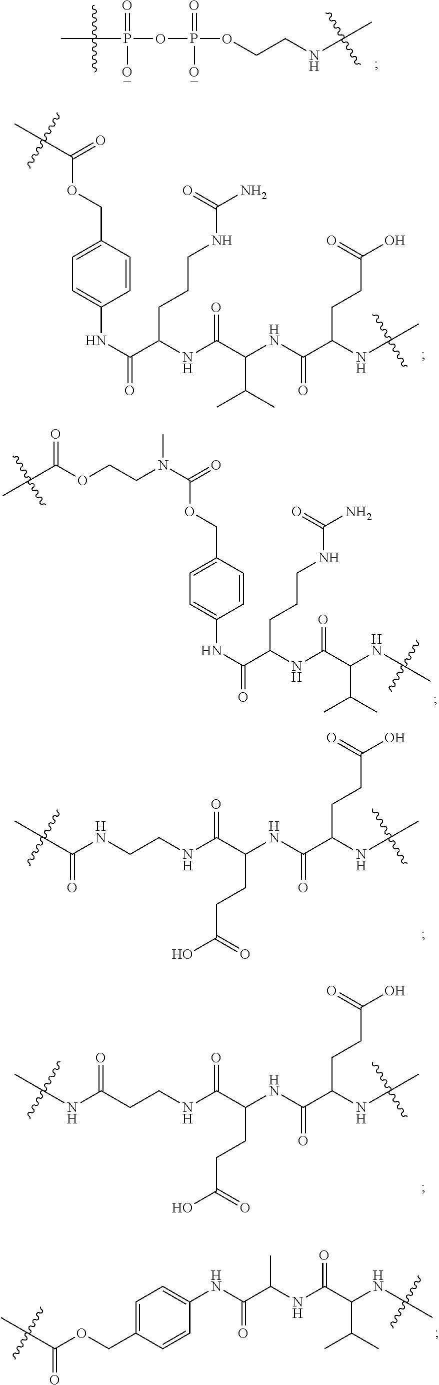

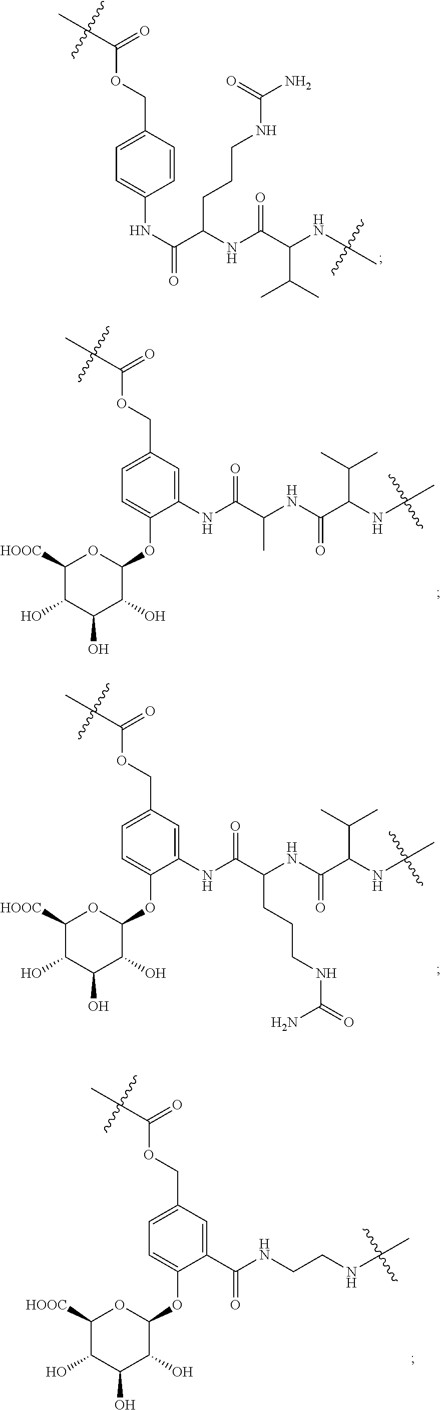 [SMILES: *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C ; *NCCNC(=O)c1cc(COC(*)=O)ccc1O[C@@H]1O[C@H](C(=O)O)[C@@H](O)[C@H](O)[C@H]1O]

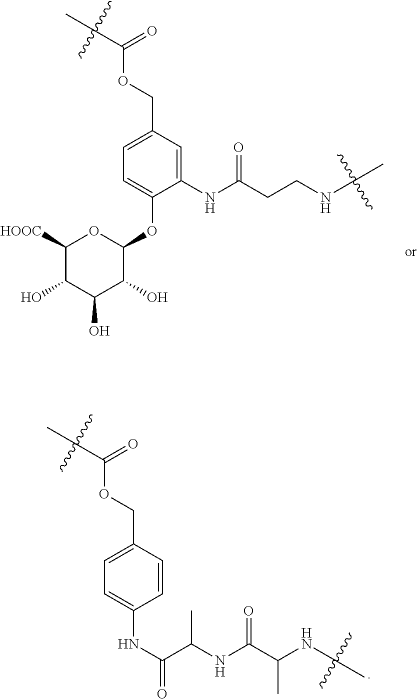 [SMILES: *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In some embodiments, the isoindolinone-glutarimide linker compound has one of the following structures:

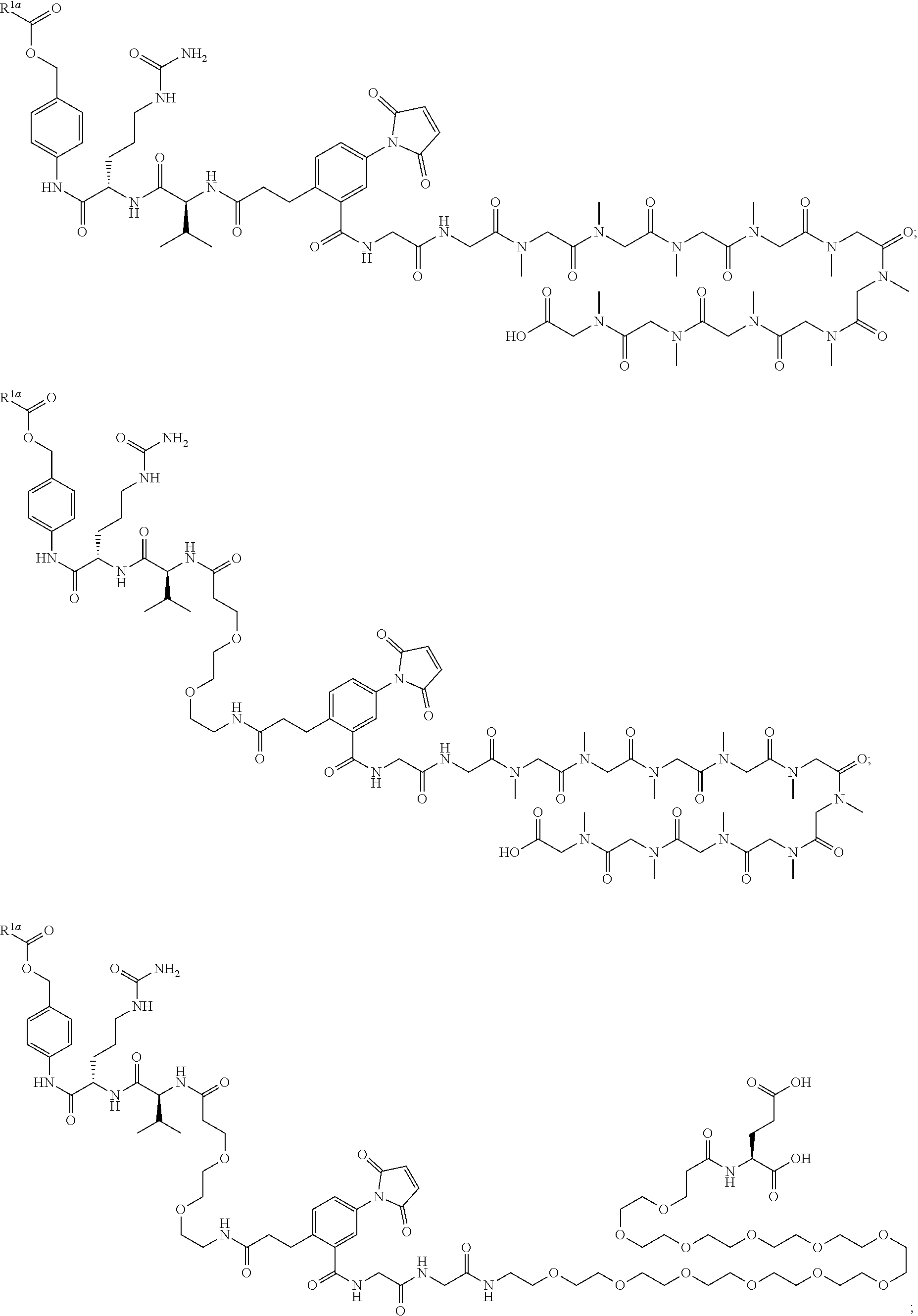

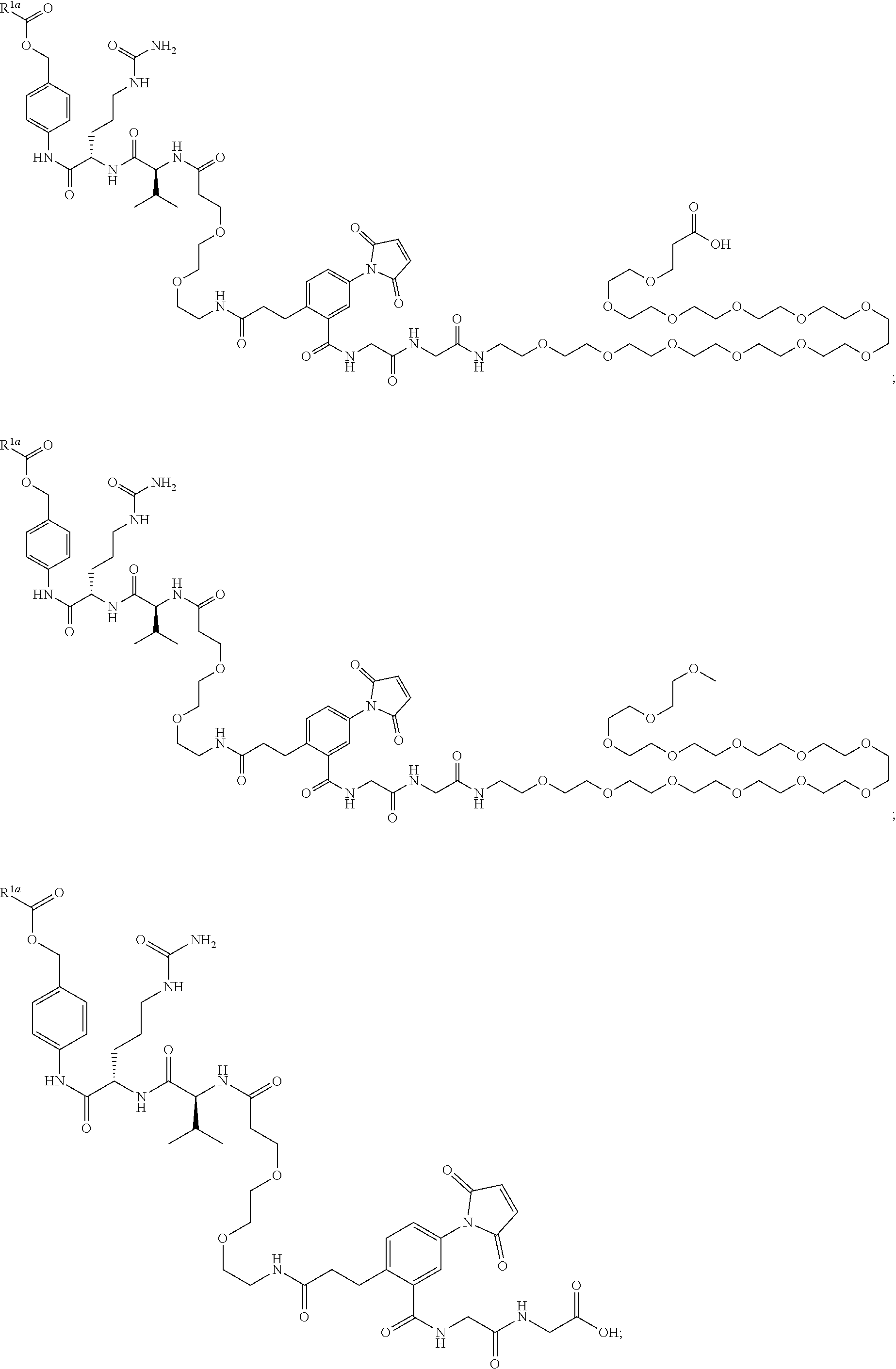

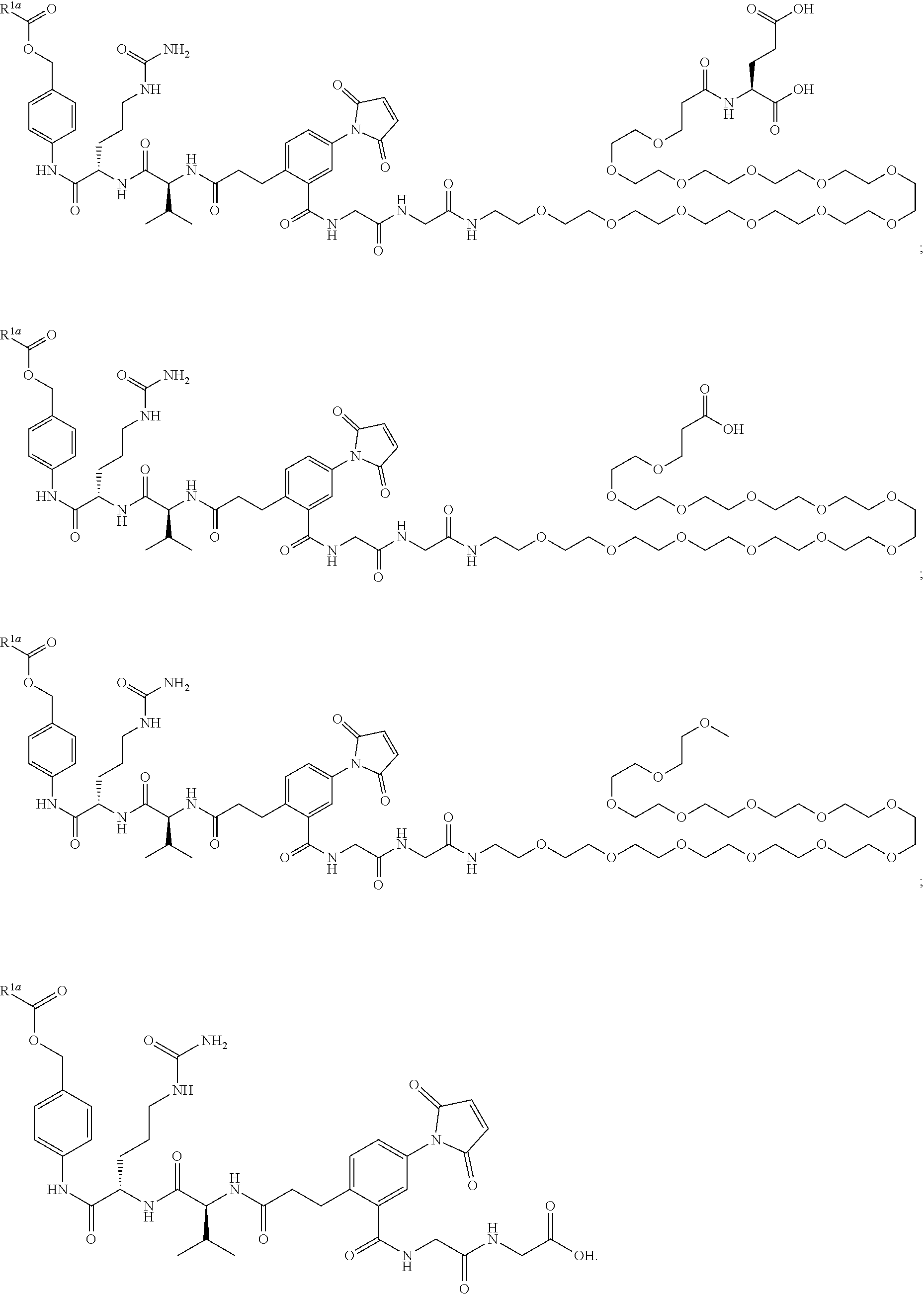

In some embodiments, the isoindolinone-glutarimide linker compound has one of the following structures:

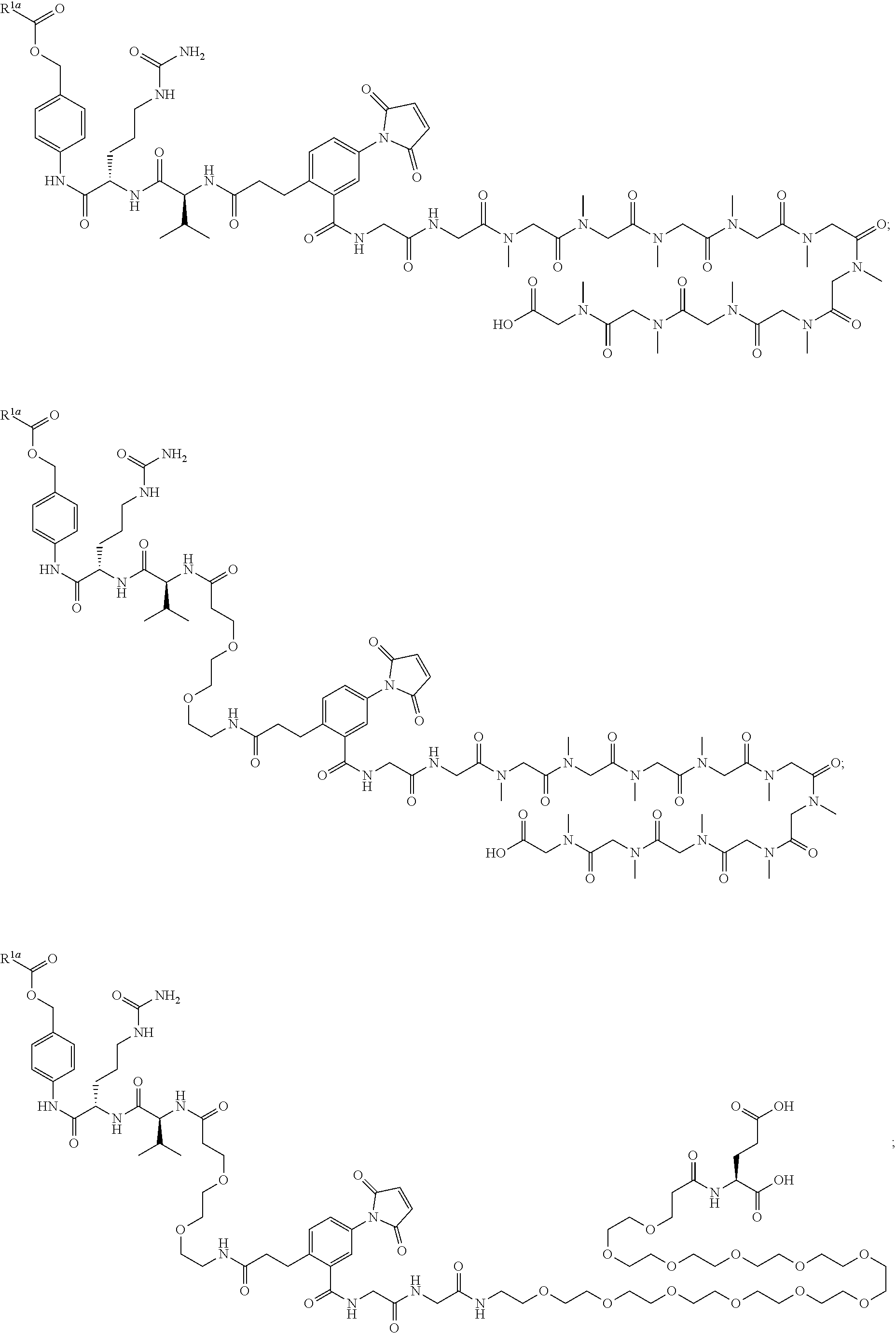

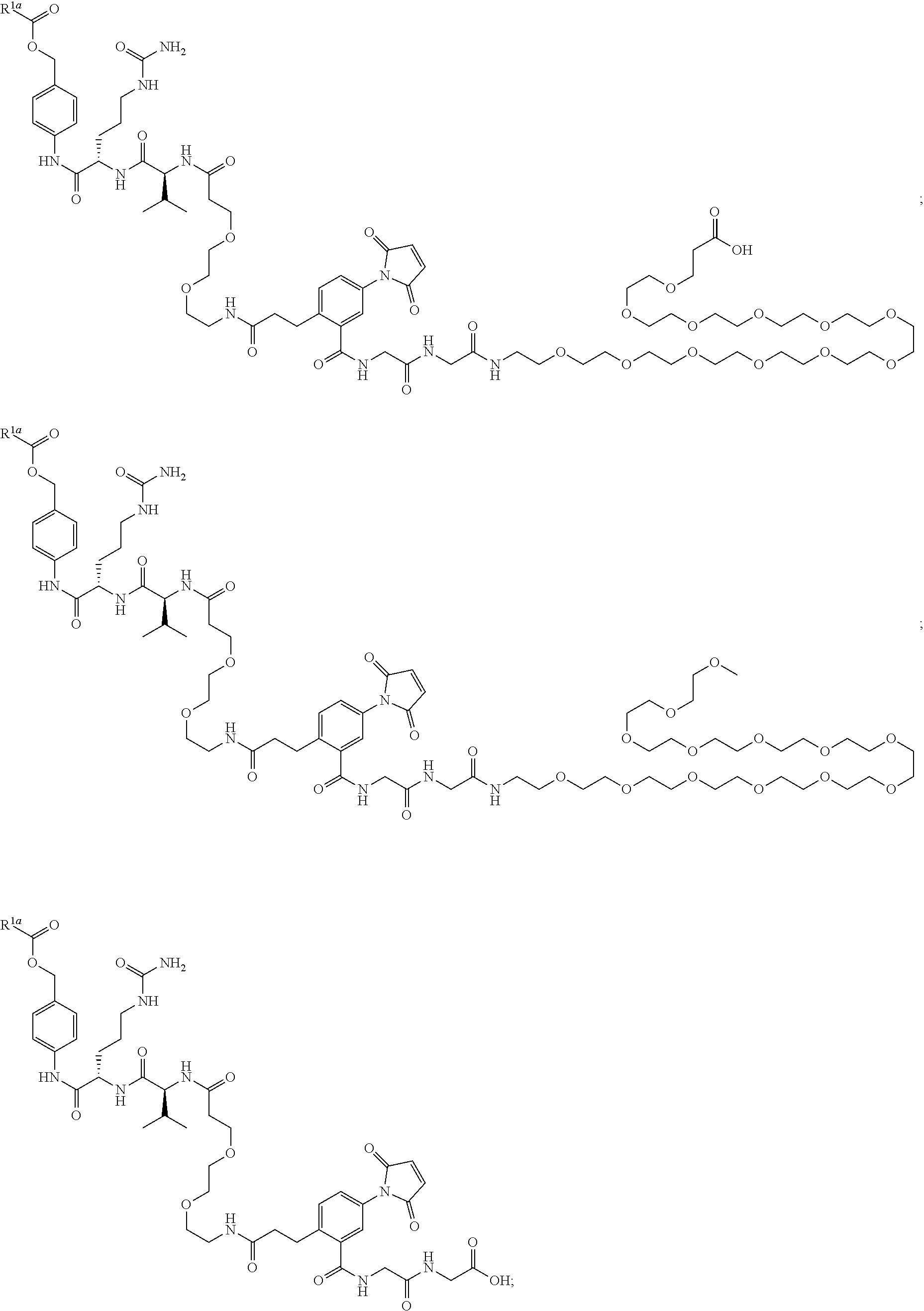

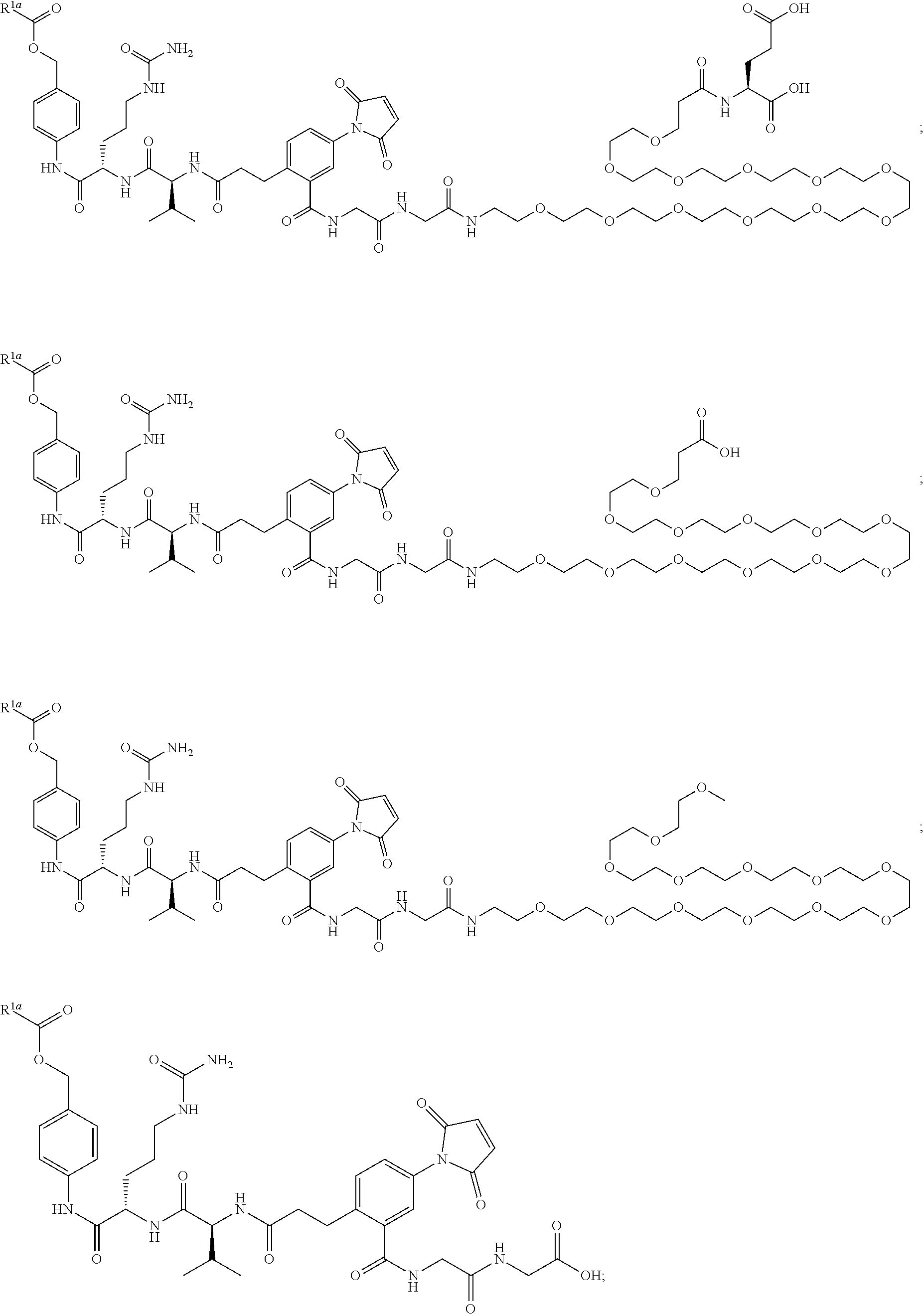

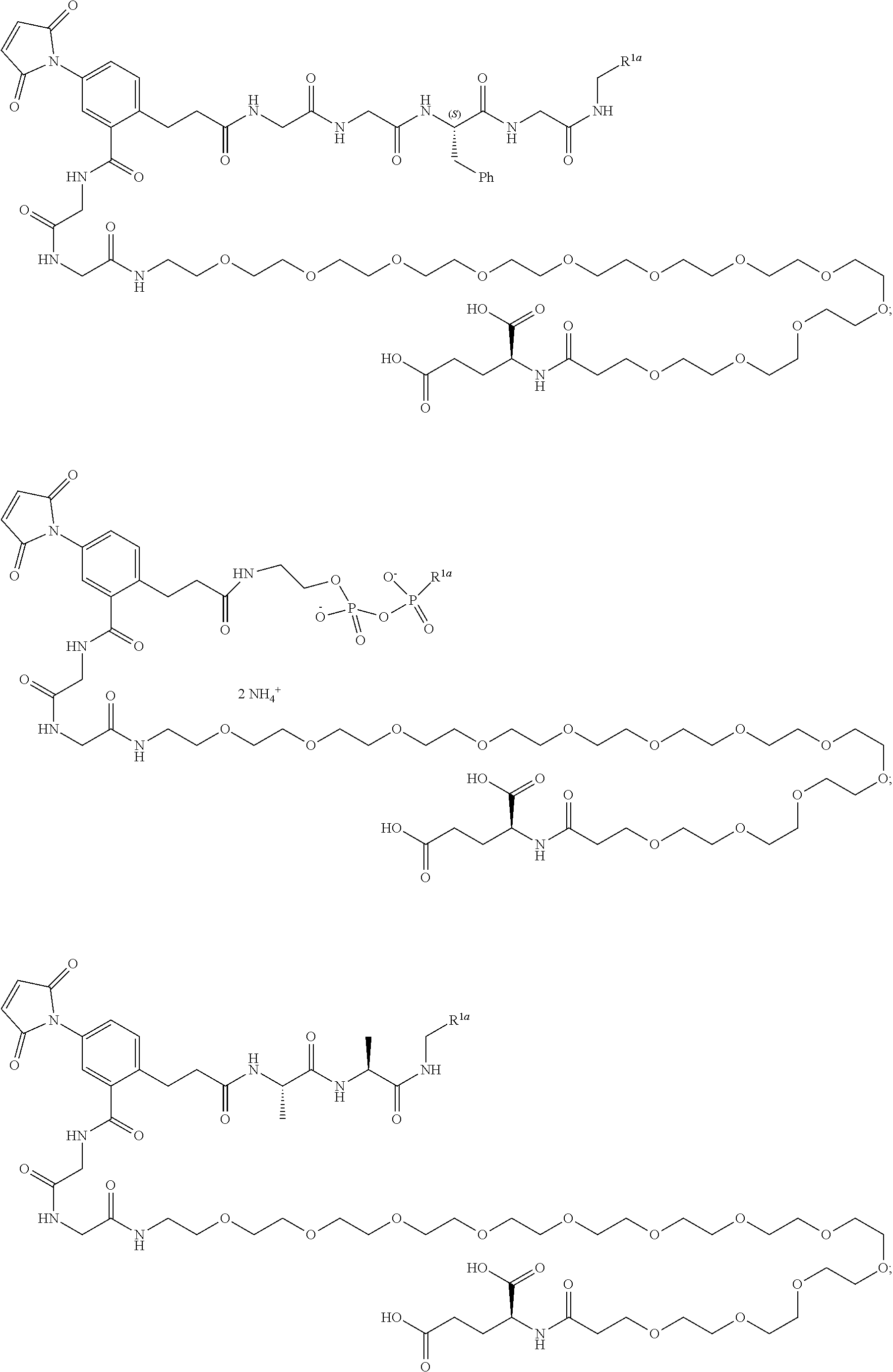

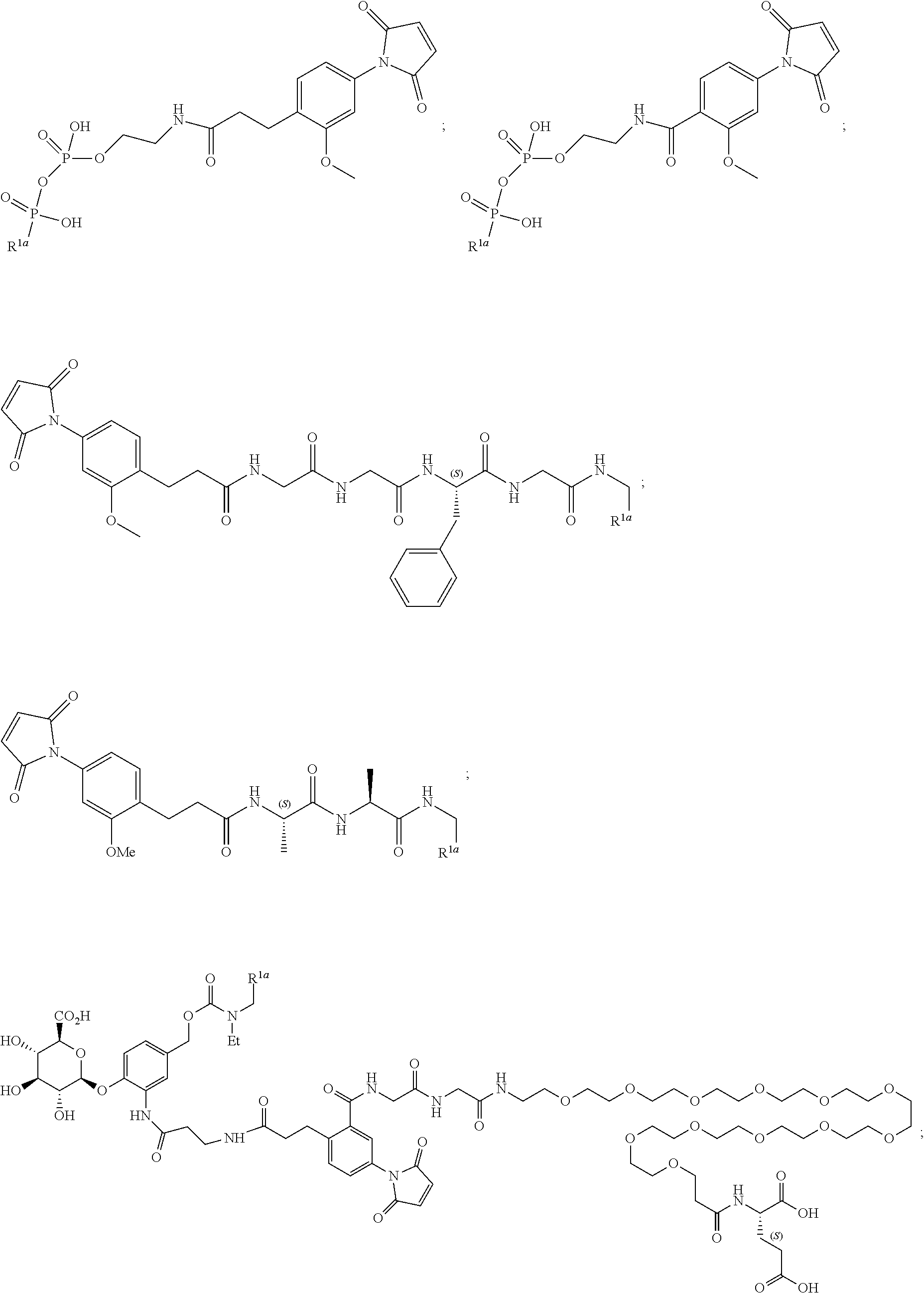

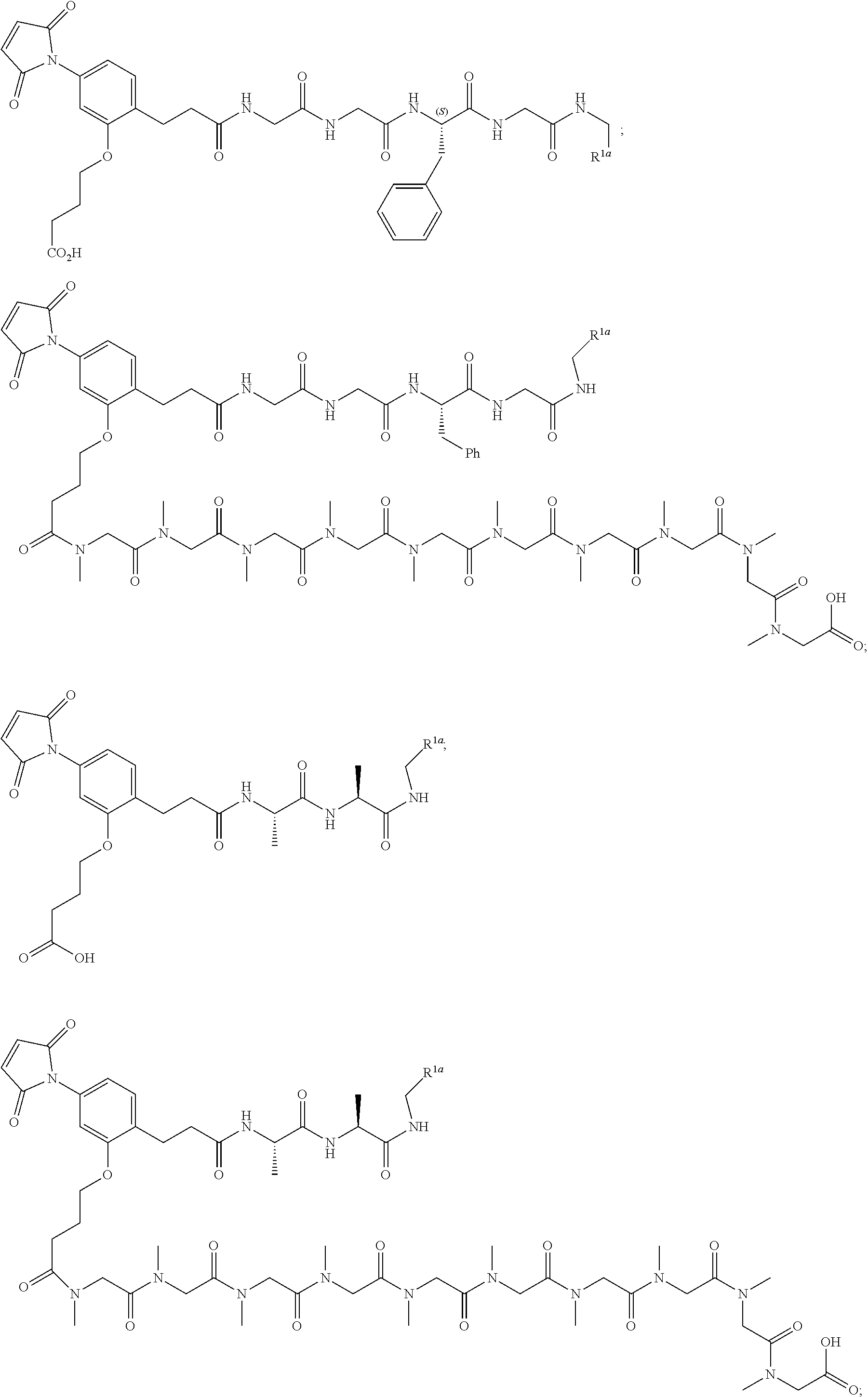

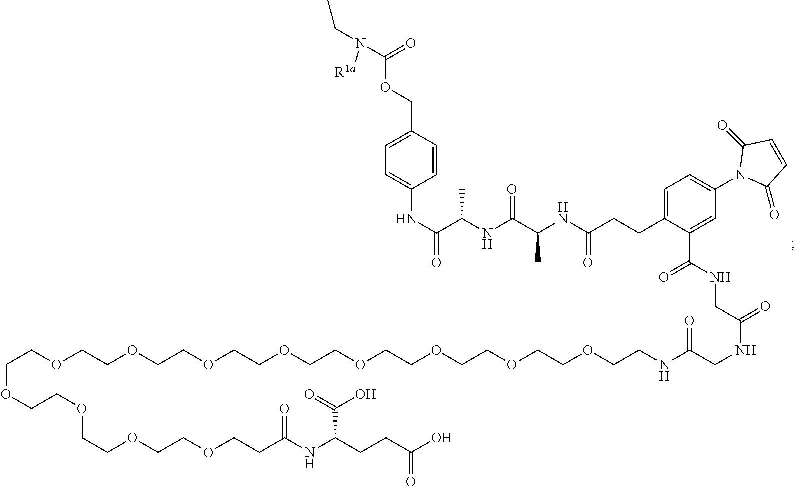

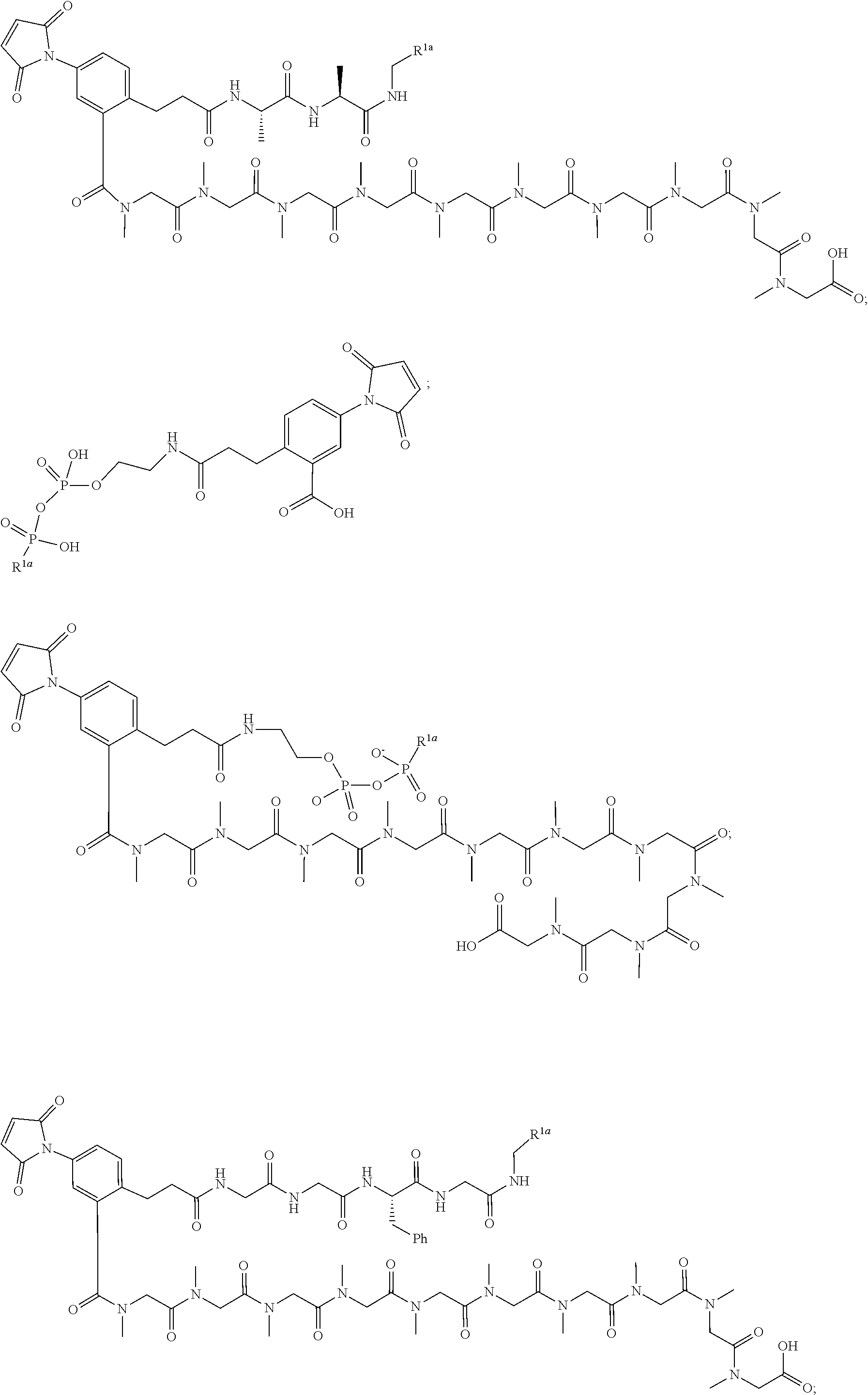

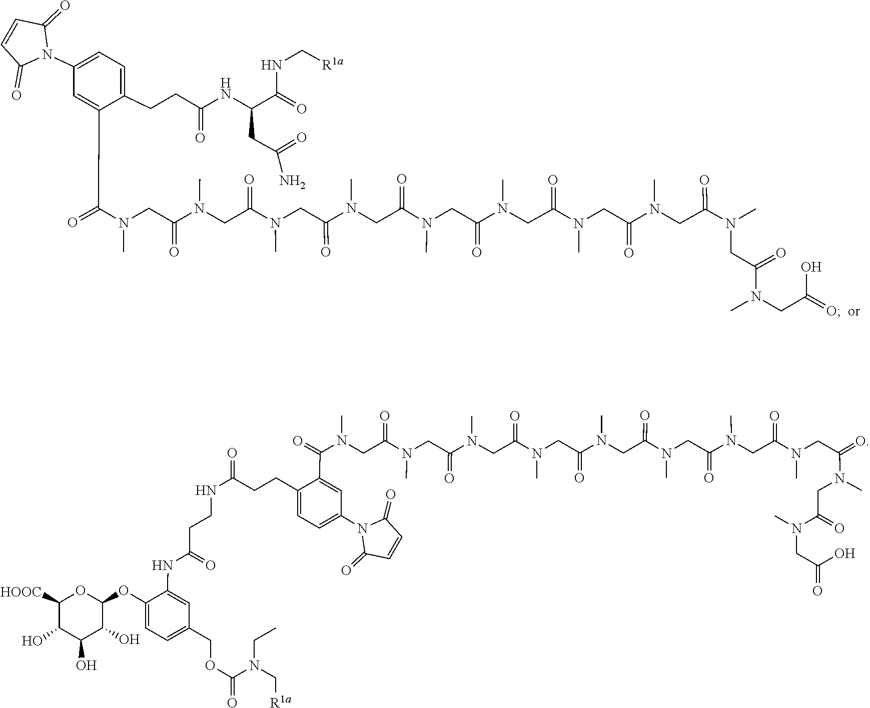

In some embodiments, the isoindolinone-glutarimide linker compound has one of the following structures:

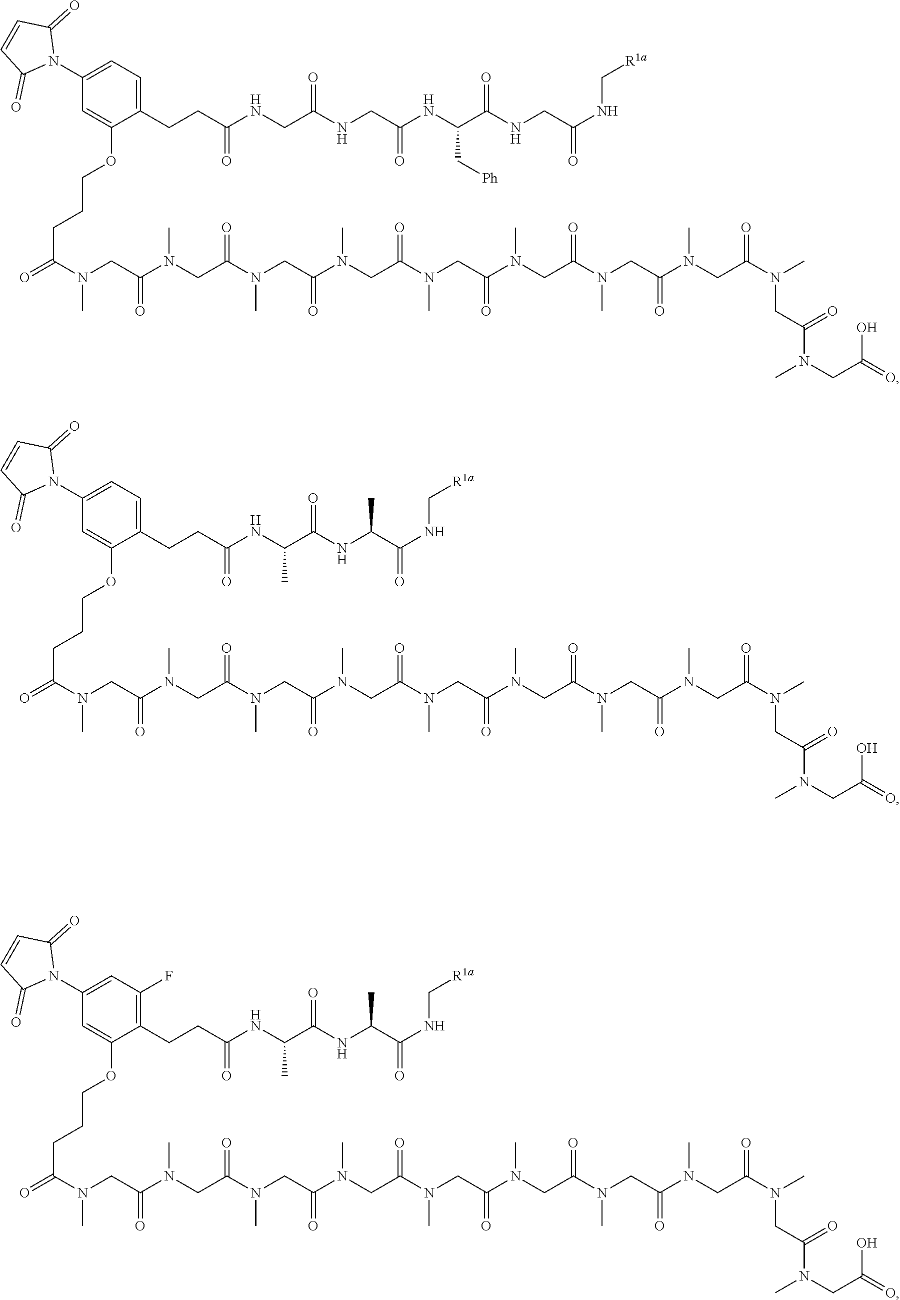

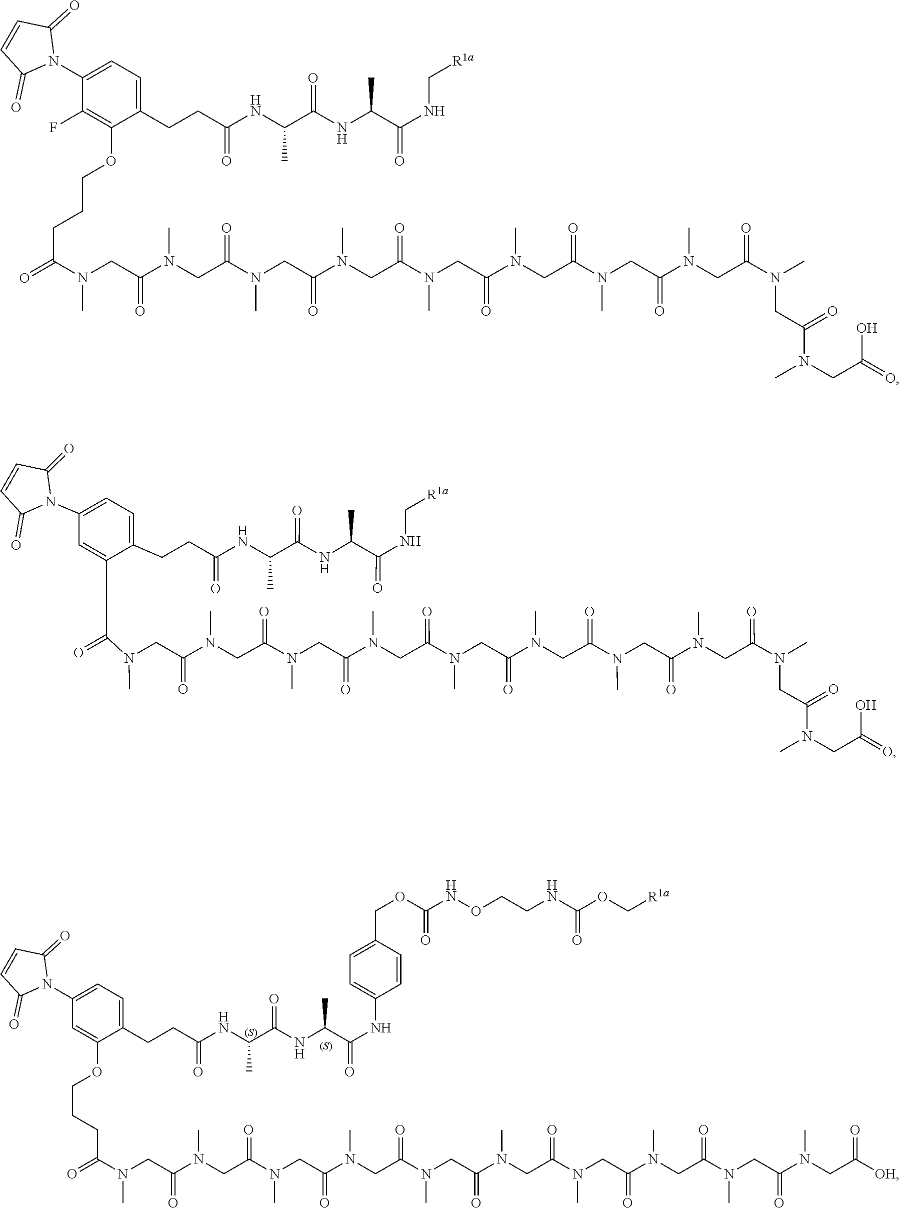

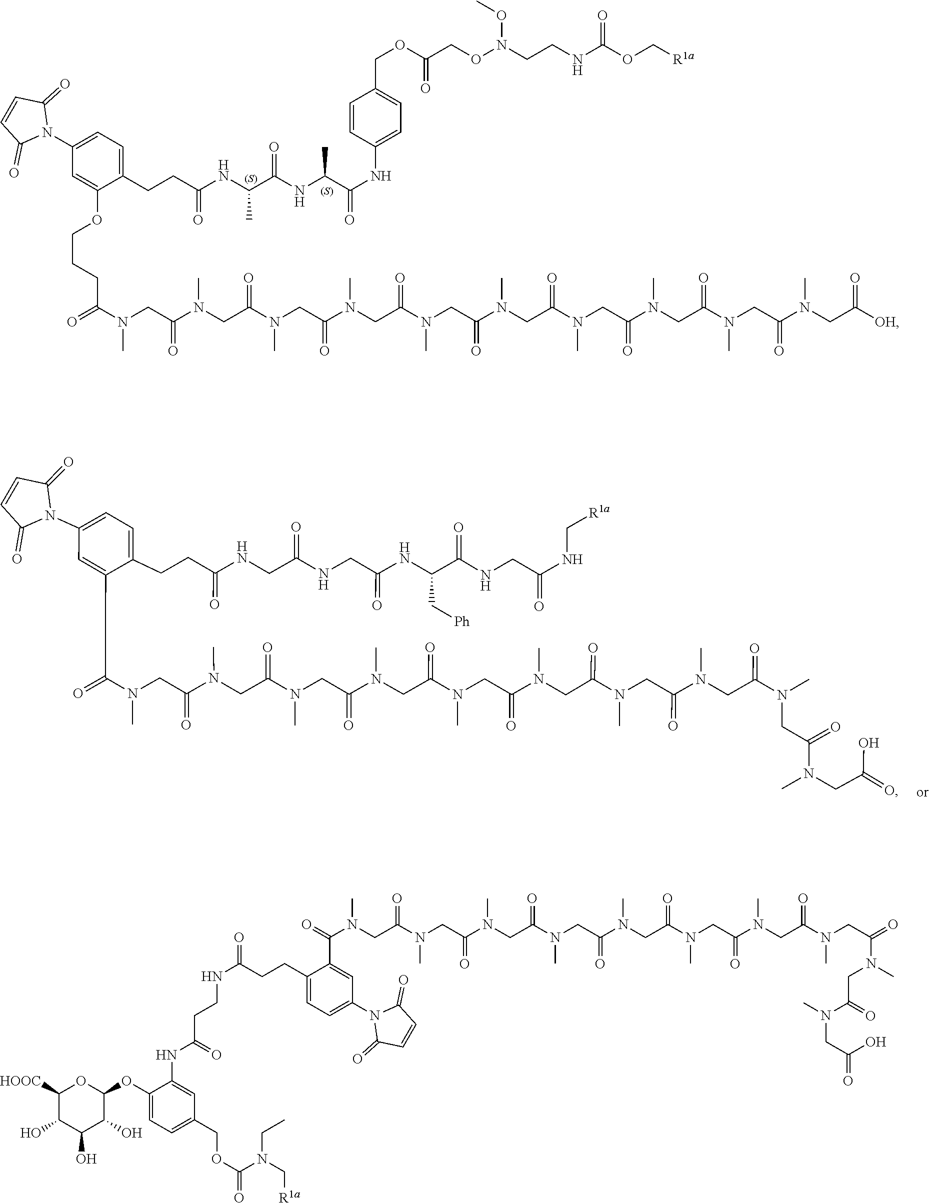

as a stereoisomer, enantiomer or tautomer thereof or a mixture thereof; or a pharmaceutically acceptable salt, solvate or prodrug thereof.

Exemplary embodiments of isoindolinone-glutarimide linker compounds (IG-L) are shown in Table 2. Each IG-L compound was prepared and characterized by mass spectrometry and shown to have the mass indicated. The IG-L compounds of Table 2 demonstrate the surprising and unexpected property of targeted protein degradation which may predict useful therapeutic activity to treat cancer and other disorders when conjugated to an antibody.

TABLE 2
Isoindolinone-glutarimide linker compounds (IG-L)

| IG-L | | |
|---|---|---|
| No. | Structure | MW |
| IG-L-1 | 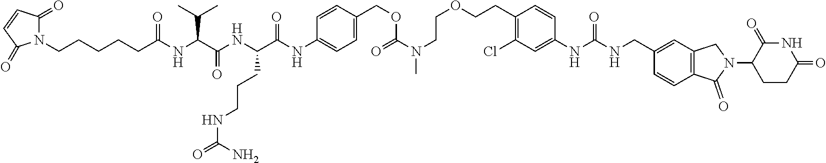 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1] | 1126.7 |
| IG-L-2 | 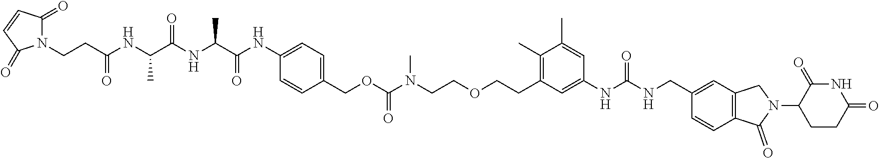 [SMILES: Cc1cc(NC(=O)NCc2ccc3c(c2)CN(C2CCC(=O)NC2=O)C3=O)cc(CCOCCN(C)C(=O)OCc2ccc(NC(=O)[C@H](C)NC(=O)[C@H](C)NC(=O)CCN3C(=O)C=CC3=O)cc2)c1C] | 964.0 |
| IG-L-3 | 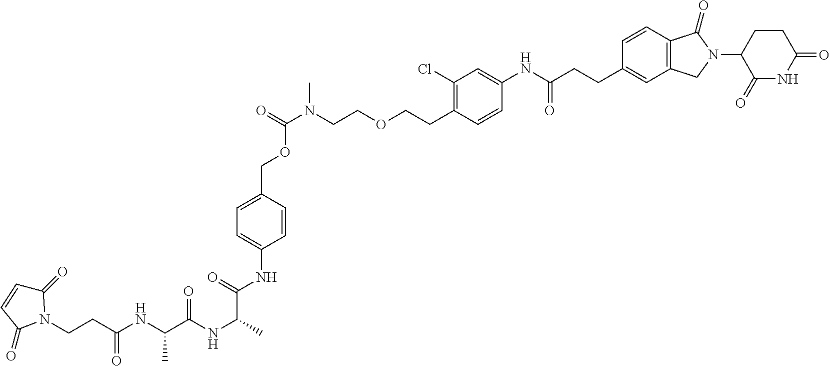 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)CCc3ccc4c(c3)CN(C3CCC(=O)NC3=O)C4=O)cc2Cl)cc1] | 969.4 |
| IG-L-4 | 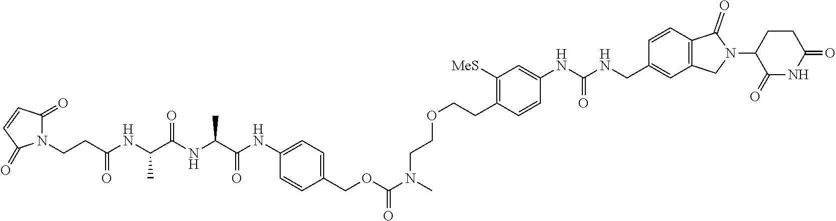 [SMILES: CSc1cc(NC(=O)NCc2ccc3c(c2)CN(C2CCC(=O)NC2=O)C3=O)ccc1CCOCCN(C)C(=O)OCc1ccc(NC(=O)[C@H](C)NC(=O)[C@H](C)NC(=O)CCN2C(=O)C=CC2=O)cc1] | 982.1 |
| IG-L-5 | 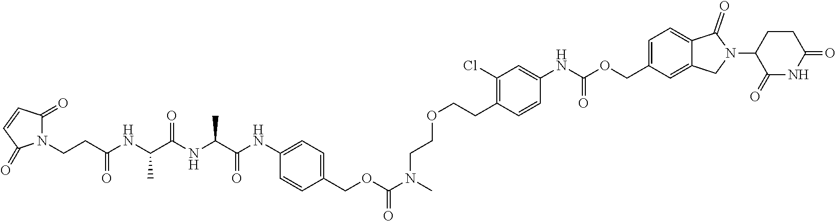 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)OCc3ccc4c(c3)CN(C3CCC(=O)NC3=O)C4=O)cc2Cl)cc1] | 971.4 |
| IG-L-6 | 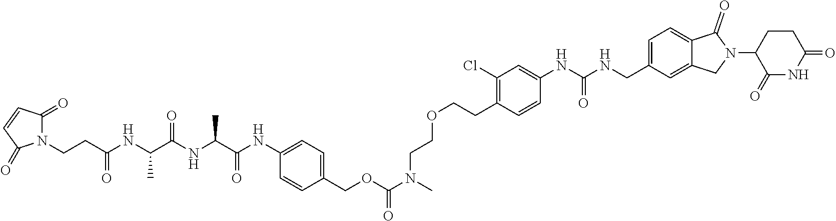 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)NCc3ccc4c(c3)CN(C3CCC(=O)NC3=O)C4=O)cc2Cl)cc1] | 970.4 |
| IG-L-7 | 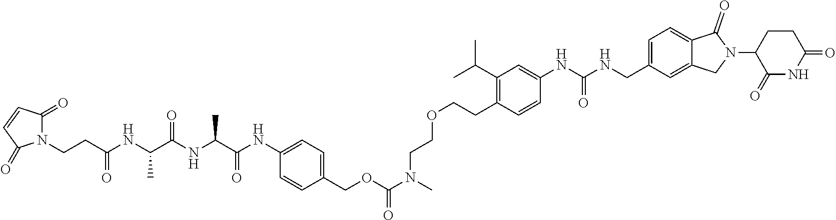 [SMILES: CC(C)c1cc(NC(=O)NCc2ccc3c(c2)CN(C2CCC(=O)NC2=O)C3=O)ccc1CCOCCN(C)C(=O)OCc1ccc(NC(=O)[C@H](C)NC(=O)[C@H](C)NC(=O)CCN2C(=O)C=CC2=O)cc1] | 978.1 |
| IG-L-8 | 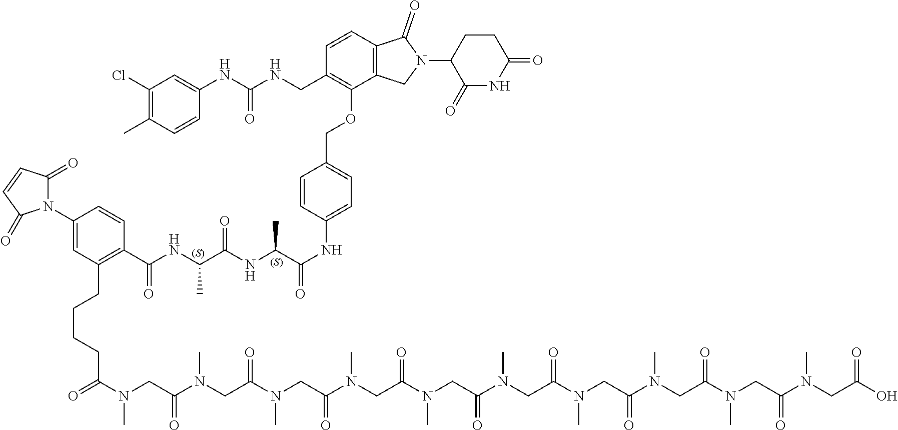 | 1744.3 |
| IG-L-9 | 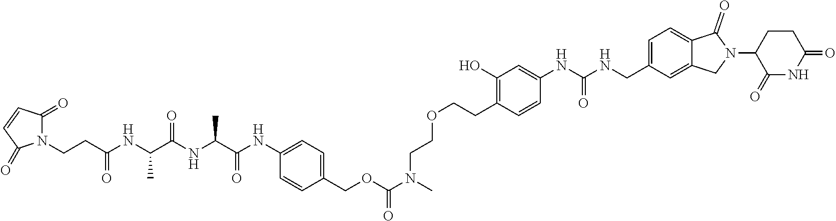 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)NCc3ccc4c(c3)CN(C3CCC(=O)NC3=O)C4=O)cc2O)cc1] | 952.0 |
| IG-L-10 | 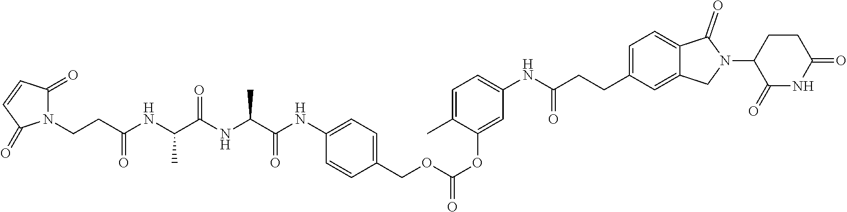 [SMILES: Cc1ccc(NC(=O)CCc2ccc3c(c2)CN(C2CCC(=O)NC2=O)C3=O)cc1OC(=O)OCc1ccc(NC(=O)[C@H](C)NC(=O)[C@H](C)NC(=O)CCN2C(=O)C=CC2=O)cc1] | 865.9 |
| IG-L-11 | 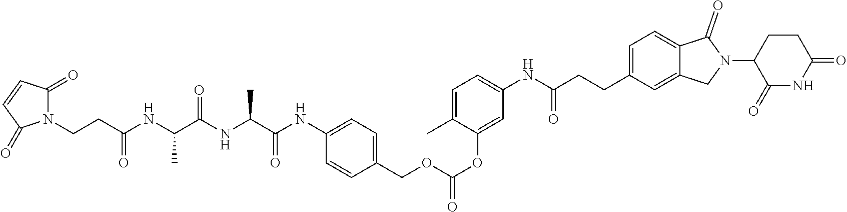 [SMILES: Cc1ccc(NC(=O)CCc2ccc3c(c2)CN(C2CCC(=O)NC2=O)C3=O)cc1OC(=O)OCc1ccc(NC(=O)[C@H](C)NC(=O)[C@H](C)NC(=O)CCN2C(=O)C=CC2=O)cc1] | 863.9 |
| IG-L-12 | 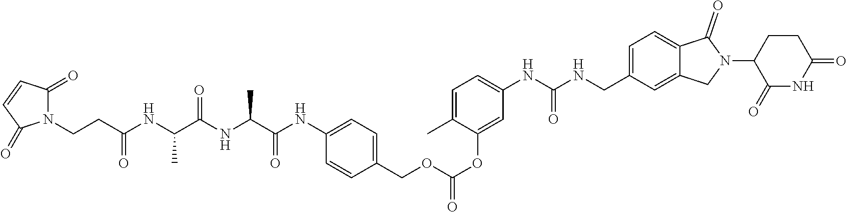 [SMILES: Cc1ccc(NC(=O)NCc2ccc3c(c2)CN(C2CCC(=O)NC2=O)C3=O)cc1OC(=O)OCc1ccc(NC(=O)[C@H](C)NC(=O)[C@H](C)NC(=O)CCN2C(=O)C=CC2=O)cc1] | 864.9 |
| IG-L-13 | 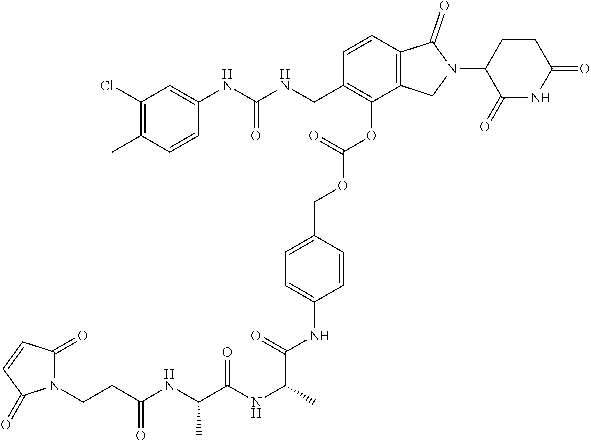 [SMILES: Cc1ccc(NC(=O)NCc2ccc3c(c2OC(=O)OCc2ccc(NC(=O)[C@H](C)NC(=O)[C@H](C)NC(=O)CCN4C(=O)C=CC4=O)cc2)CN(C2CCC(=O)NC2=O)C3=O)cc1Cl] | 899.3 |
| IG-L-14 | 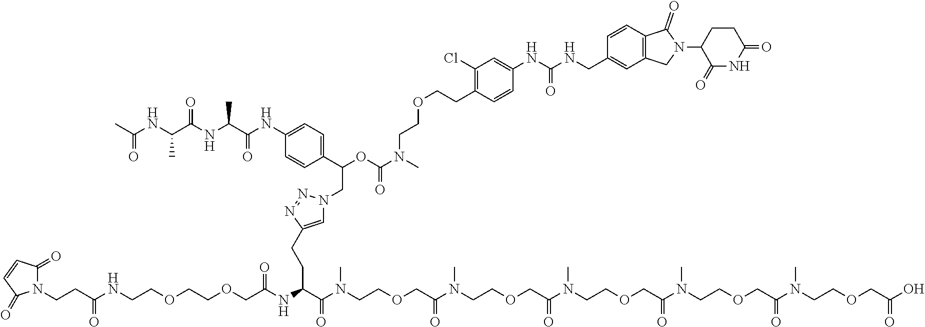 [SMILES: CC(=O)N[C@@H](C)C(=O)N[C@@H](C)C(=O)Nc1ccc(C(Cn2cc(CC[C@H](NC(=O)COCCOCCNC(=O)CCN3C(=O)C=CC3=O)C(=O)N(C)CCOCC(=O)N(C)CCOCC(=O)N(C)CCOCC(=O)N(C)CCOCC(=O)N(C)CCOCC(=O)O)nn2)OC(=O)N(C)CCOCCc2ccc(NC(=O)NCc3ccc4c(c3)CN(C3CCC(=O)NC3=O)C4=O)cc2Cl)cc1] | 1915.5 |
| IG-L-15 | 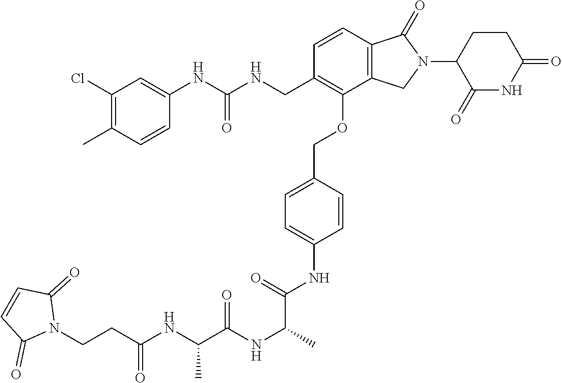 [SMILES: Cc1ccc(NC(=O)NCc2ccc3c(c2OCc2ccc(NC(=O)[C@H](C)NC(=O)[C@H](C)NC(=O)CCN4C(=O)C=CC4=O)cc2)CN(C2CCC(=O)NC2=O)C3=O)cc1Cl] | 855.3 |
| IG-L-16 | 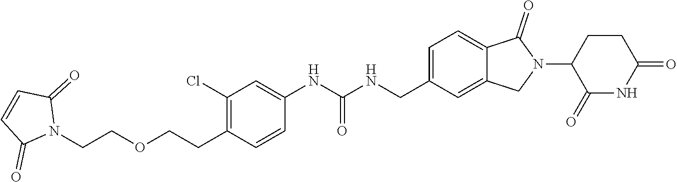 [SMILES: O=C1CCC(N2Cc3cc(CNC(=O)Nc4ccc(CCOCCN5C(=O)C=CC5=O)c(Cl)c4)ccc3C2=O)C(=O)N1] | 594.0 |
| IG-L-17 | 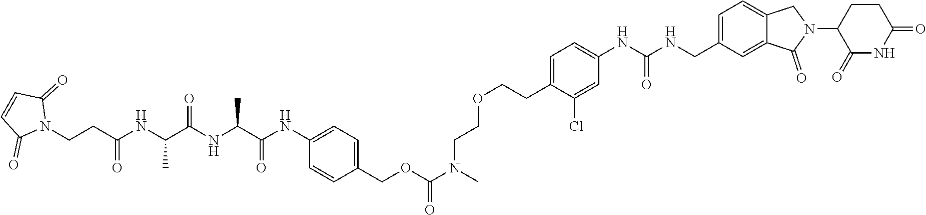 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)NCc3ccc4c(c3)C(=O)N(C3CCC(=O)NC3=O)C4)cc2Cl)cc1] | 970.4 |
| IG-L-18 | 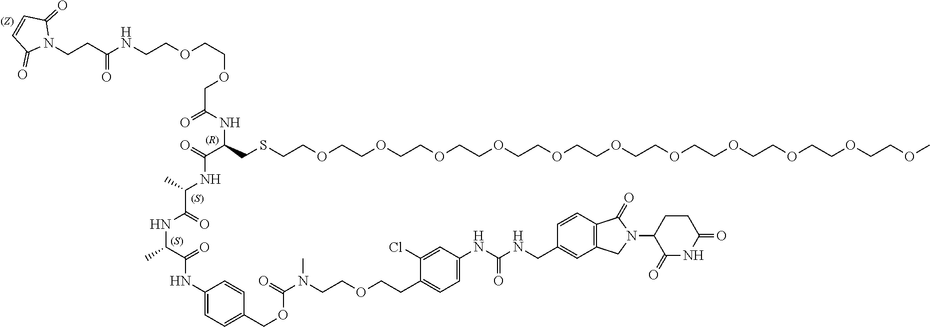 | 1717.3 |
| IG-L-19 | 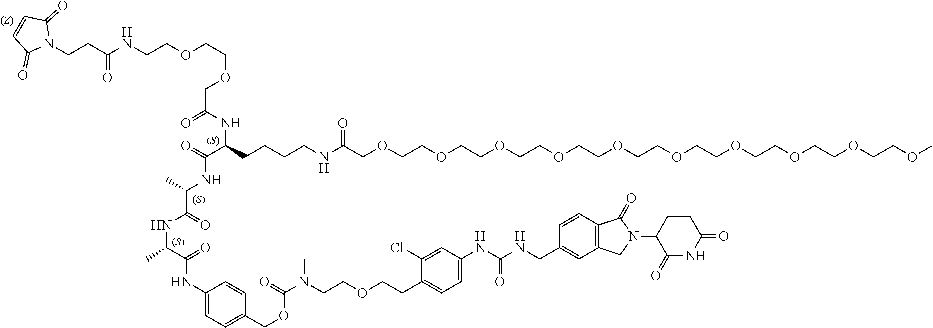 | 1712.3 |
| IG-L-20 | 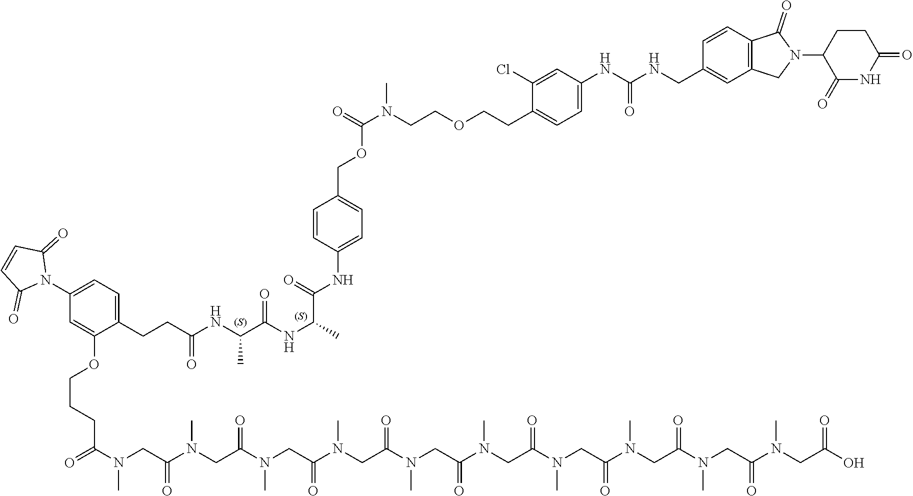 | 1859.4 |
| IG-L-21 | 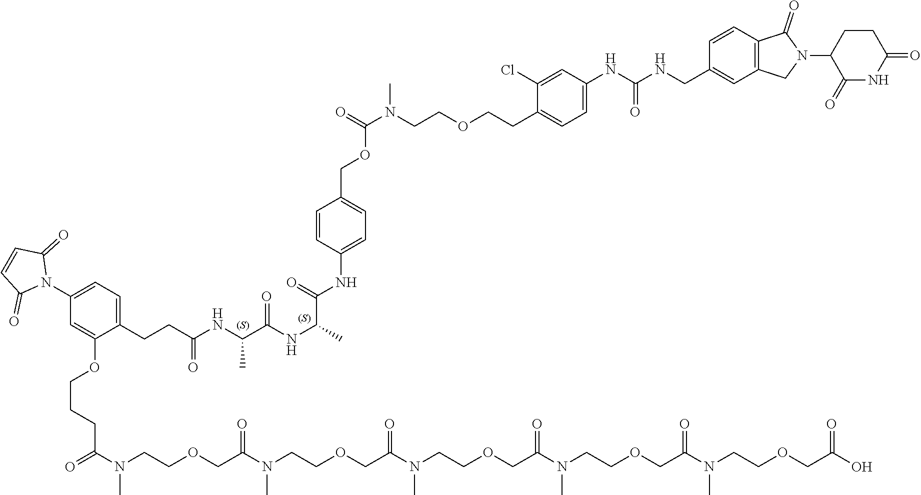 | 1724.3 |
| IG-L-22 | 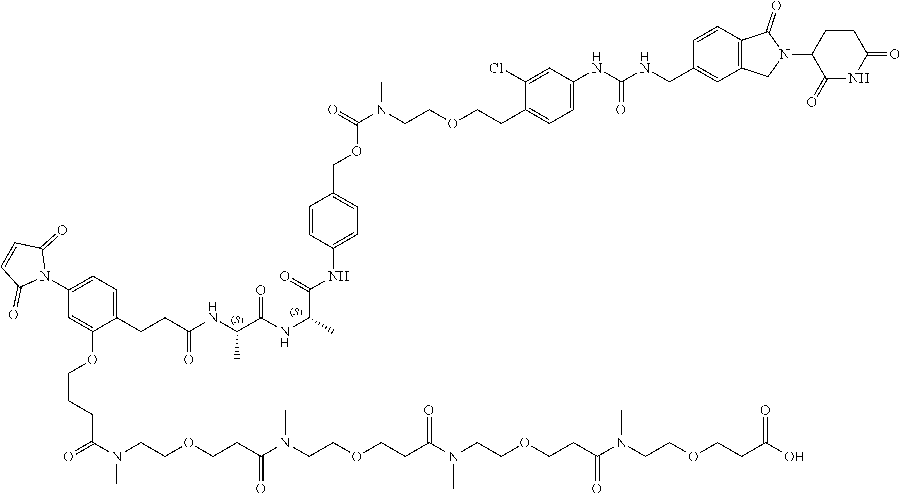 | 1665.3 |
| IG-L-23 | 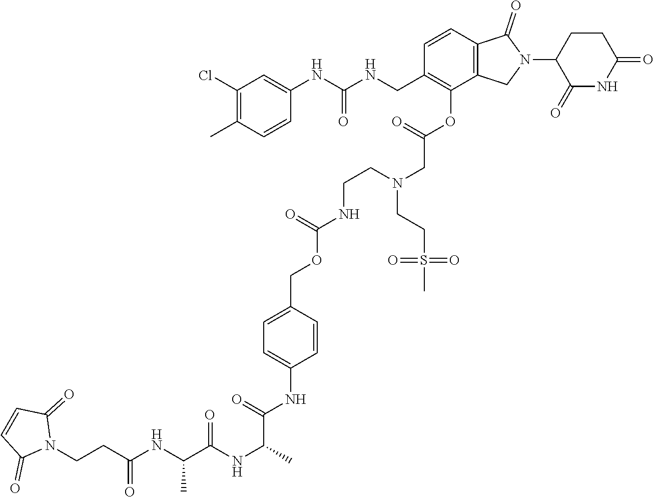 [SMILES: Cc1ccc(NC(=O)NCc2ccc3c(c2OC(=O)CN(CCNC(=O)OCc2ccc(NC(=O)[C@H](C)NC(=O)[C@H](C)NC(=O)CCN4C(=O)C=CC4=O)cc2)CCS(C)(=O)=O)CN(C2CCC(=O)NC2=O)C3=O)cc1Cl] | 1105.6 |
| IG-L-24 | 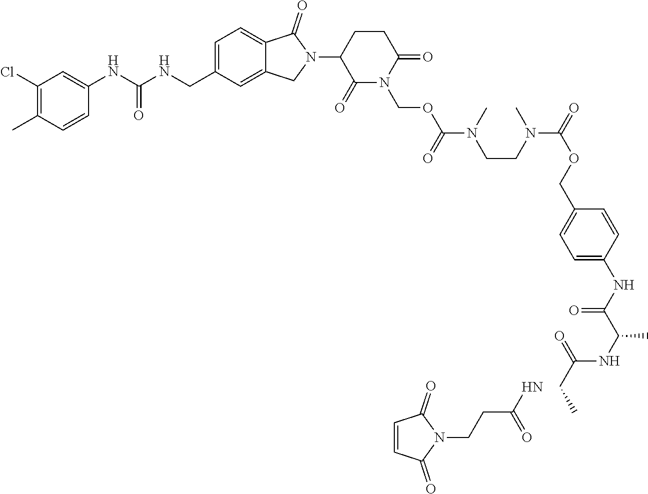 | 1027.5 |
| IG-L-25 | 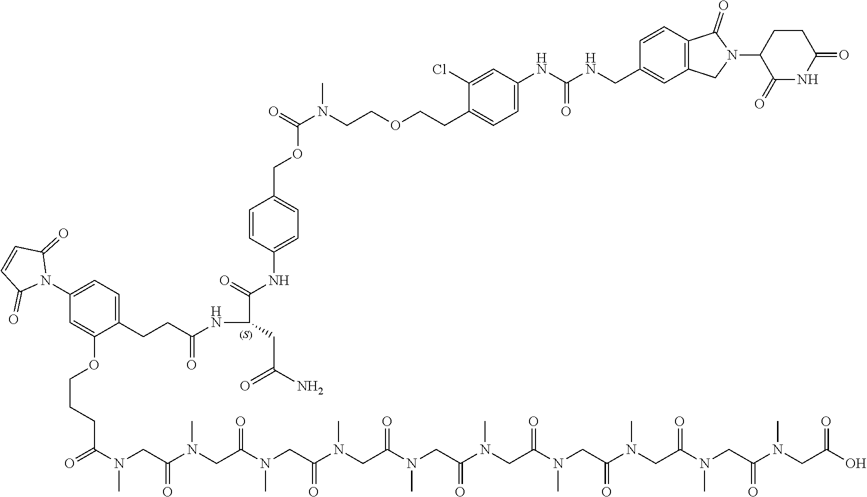 | 1831.4 |
| IG-L-26 | 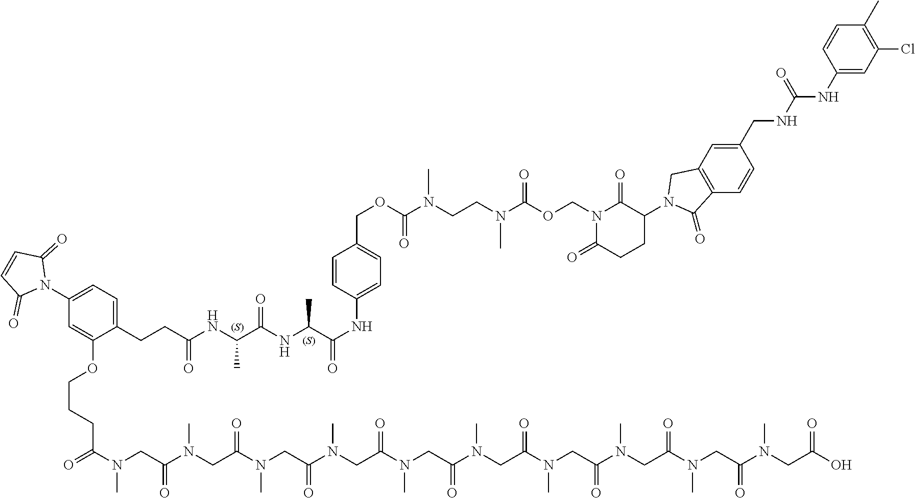 | 1916.5 |
| IG-L-27 | 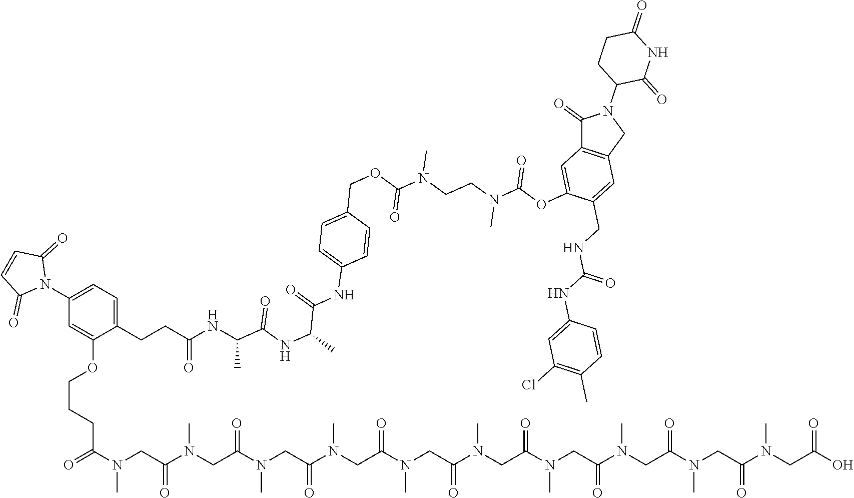 [SMILES: Cc1ccc(NC(=O)NCc2cc3c(cc2OC(=O)N(C)CCN(C)C(=O)OCc2ccc(NC(=O)[C@H](C)NC(=O)[C@H](C)NC(=O)CCc4ccc(N5C(=O)C=CC5=O)cc4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc2)C(=O)N(C2CCC(=O)NC2=O)C3)cc1Cl] | 1902.4 |
| IG-L-28 | 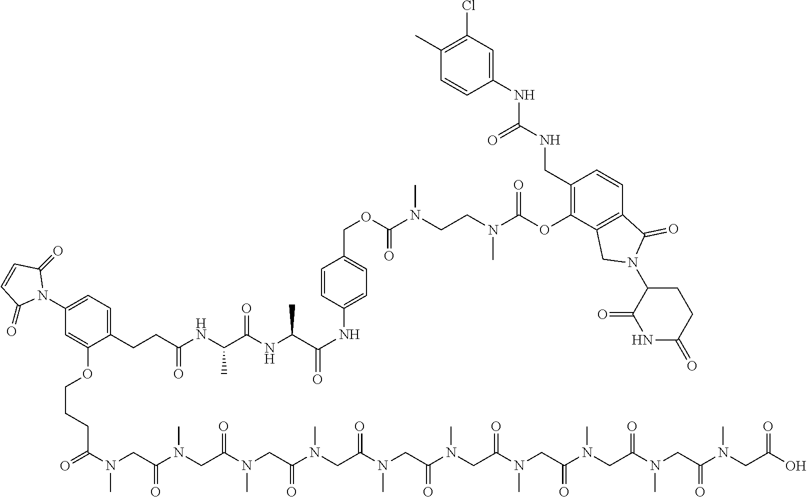 [SMILES: Cc1ccc(NC(=O)NCc2ccc3c(c2OC(=O)N(C)CCN(C)C(=O)OCc2ccc(NC(=O)[C@H](C)NC(=O)[C@H](C)NC(=O)CCc4ccc(N5C(=O)C=CC5=O)cc4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc2)CN(C2CCC(=O)NC2=O)C3=O)cc1Cl] | 1902.4 |
| IG-L-29 | 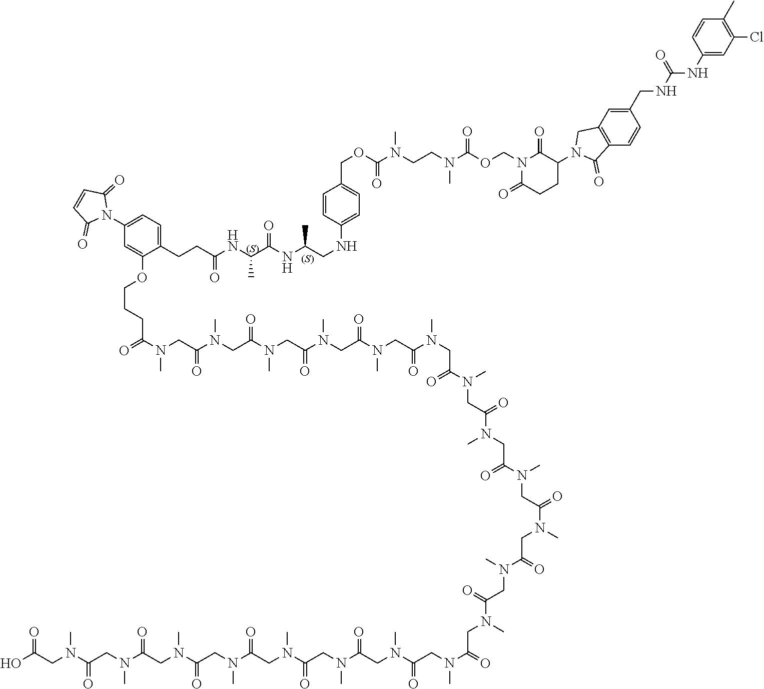 | 2627.3 |
| IG-L-30 | 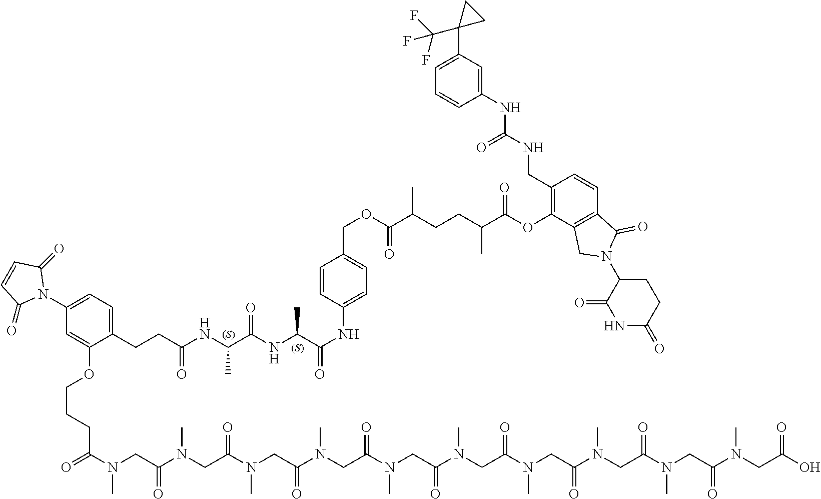 | 1962.0 |
| IG-L-31 | 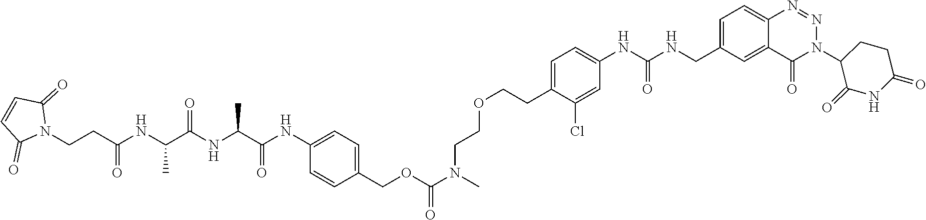 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)NCc3ccc4nnn(C5CCC(=O)NC5=O)c(=O)c4c3)cc2Cl)cc1] | 984.4 |
| IG-L-32 | 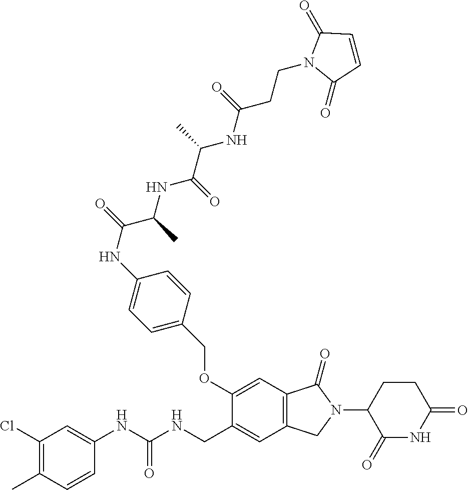 [SMILES: Cc1ccc(NC(=O)NCc2cc3c(cc2OCc2ccc(NC(=O)[C@H](C)NC(=O)[C@H](C)NC(=O)CCN4C(=O)C=CC4=O)cc2)C(=O)N(C2CCC(=O)NC2=O)C3)cc1Cl] | 855.3 |
| IG-L-33 | 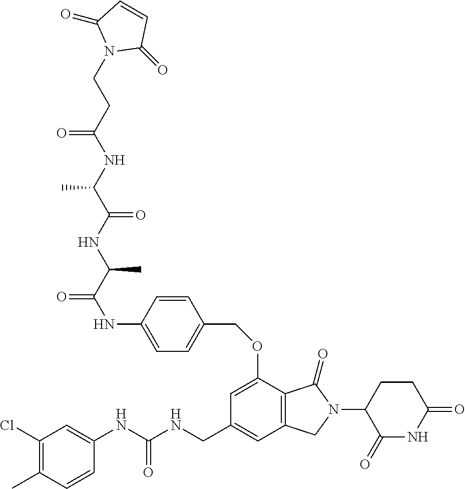 [SMILES: Cc1ccc(NC(=O)NCc2cc3c(c(OCc4ccc(NC(=O)[C@H](C)NC(=O)[C@H](C)NC(=O)CCN5C(=O)C=CC5=O)cc4)c2)C(=O)N(C2CCC(=O)NC2=O)C3)cc1Cl] | 855.3 |
| IG-L-34 | 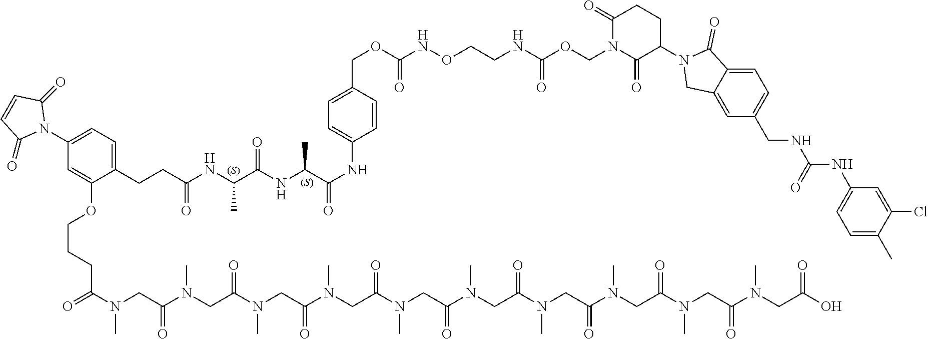 | 1902.7 |
| IG-L-35 | 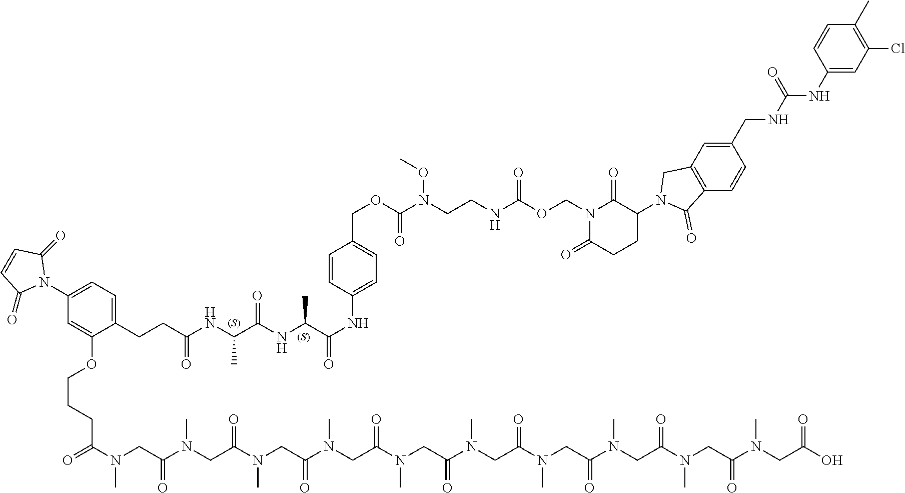 | 1916.8 |
| IG-L-36 | 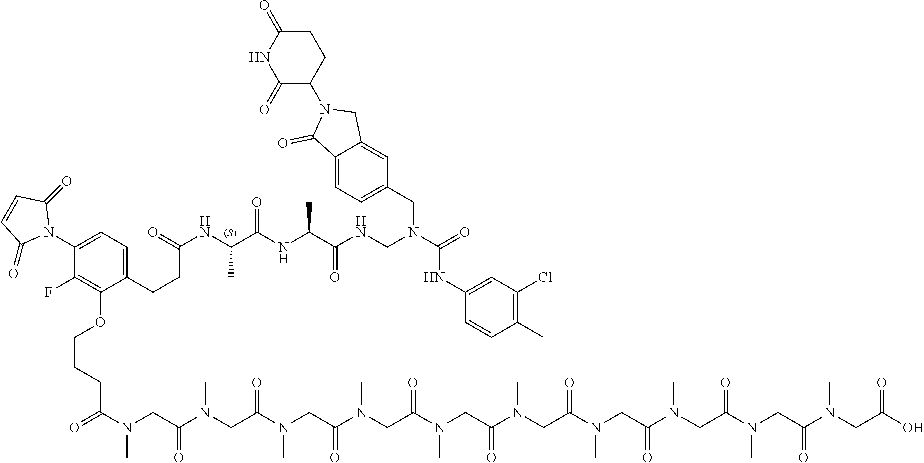 | 1670.2 |
| IG-L-37 | 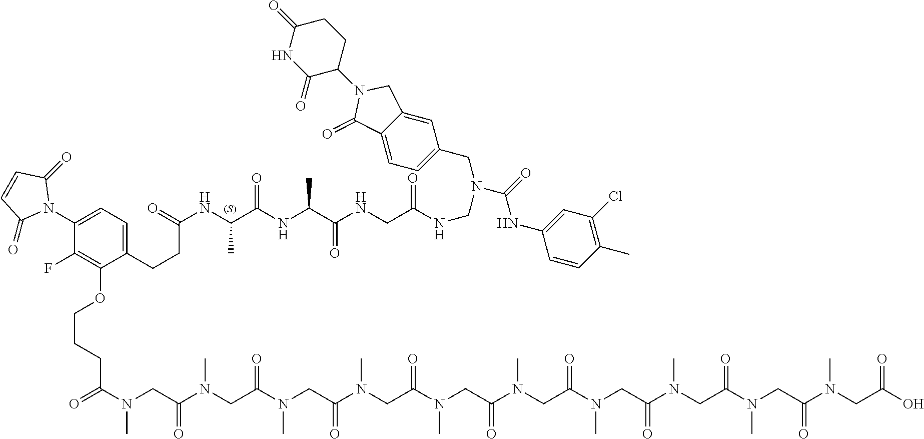 | 1727.3 |
| IG-L-38 | 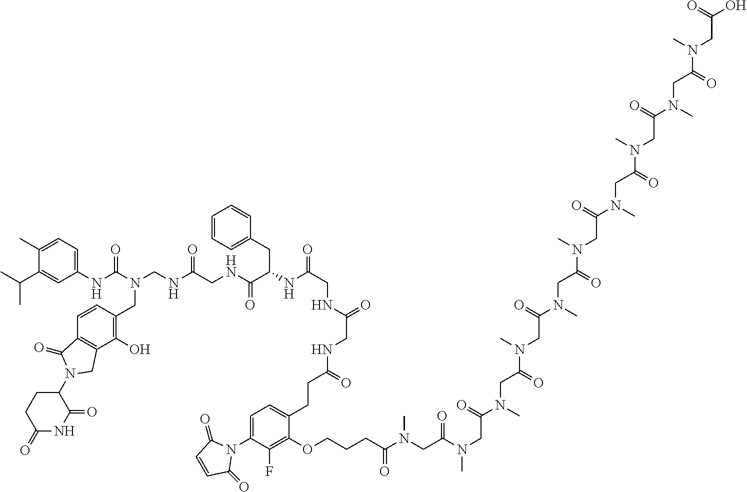 [SMILES: Cc1ccc(NC(=O)N(CNC(=O)CNC(=O)[C@H](Cc2ccccc2)NC(=O)CNC(=O)CNC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2ccc3c(c2O)CN(C2CCC(=O)NC2=O)C3=O)cc1C(C)C] | 1870.0 |
| IG-L-39 | 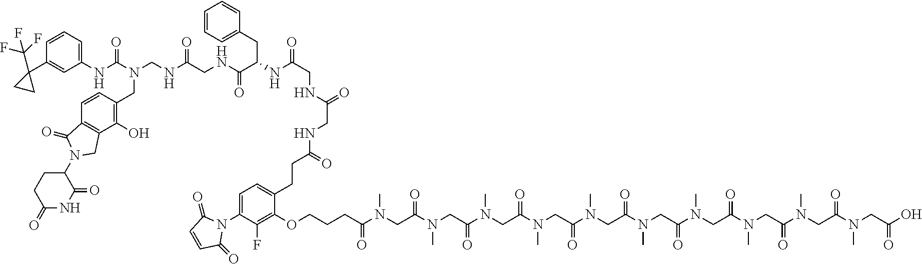 [SMILES: CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCCOc1c(CCC(=O)NCC(=O)NCC(=O)N[C@@H](Cc2ccccc2)C(=O)NCC(=O)NCN(Cc2ccc3c(c2O)CN(C2CCC(=O)NC2=O)C3=O)C(=O)Nc2cccc(C3(C(F)(F)F)CC3)c2)ccc(N2C(=O)C=CC2=O)c1F] | 1921.9 |
| IG-L-40 | 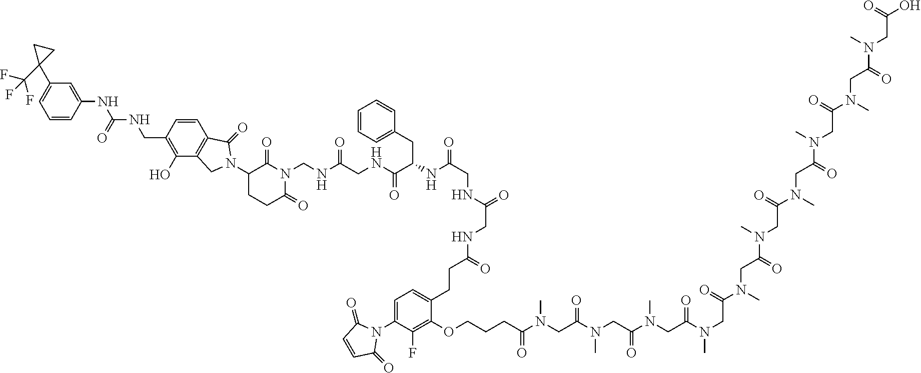 [SMILES: CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCCOc1c(CCC(=O)NCC(=O)NCC(=O)N[C@@H](Cc2ccccc2)C(=O)NCC(=O)NCN2C(=O)CCC(N3Cc4c(ccc(CNC(=O)Nc5cccc(C6(C(F)(F)F)CC6)c5)c4O)C3=O)C2=O)ccc(N2C(=O)C=CC2=O)c1F] | 1921.9 |
| IG-L-41 | 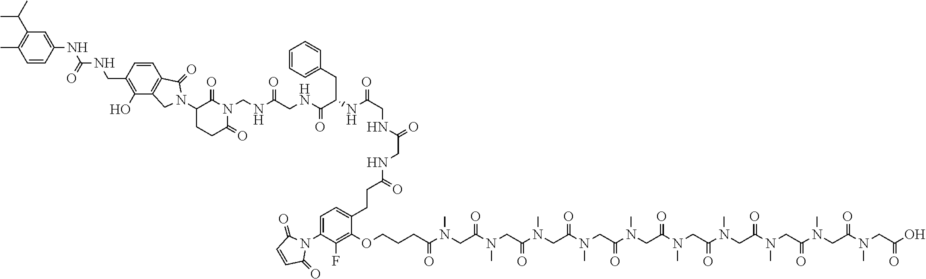 [SMILES: Cc1ccc(NC(=O)NCc2ccc3c(c2O)CN(C2CCC(=O)N(CNC(=O)CNC(=O)[C@H](Cc4ccccc4)NC(=O)CNC(=O)CNC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1C(C)C] | 1870.0 |
| IG-L-42 | 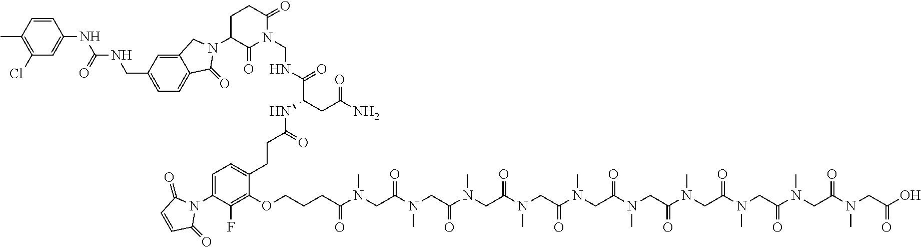 [SMILES: Cc1ccc(NC(=O)NCc2ccc3c(c2)CN(C2CCC(=O)N(CNC(=O)[C@H](CC(N)=O)NC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl] | 1642.12 |
| IG-L-43 | 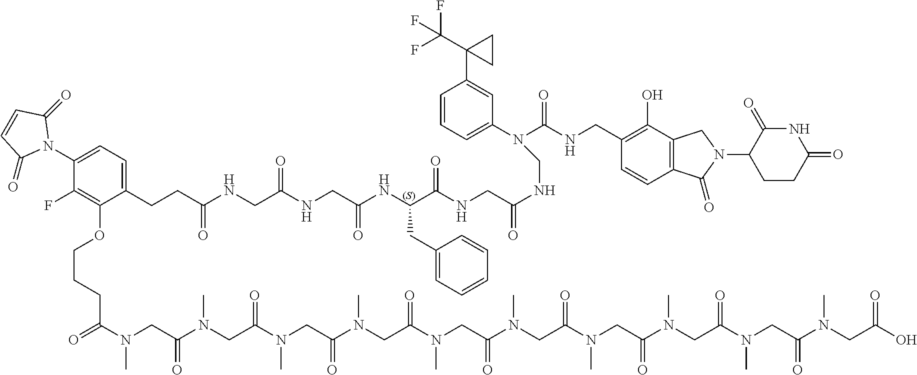 | 1921.9 |
| IG-L-44 | 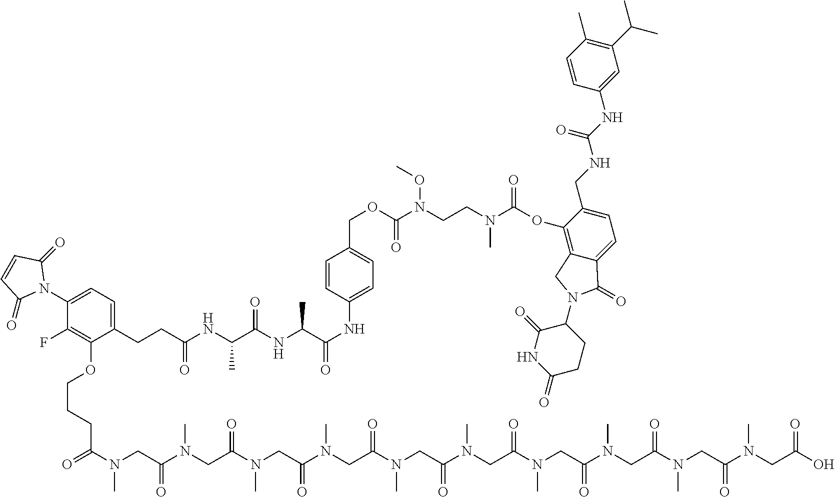 [SMILES: CON(CCN(C)C(=O)Oc1c(CNC(=O)Nc2ccc(C)c(C(C)C)c2)ccc2c1CN(C1CCC(=O)NC1=O)C2=O)C(=O)OCc1ccc(NC(=O)[C@H](C)NC(=O)[C@H](C)NC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc1] | 1944.1 |
| IG-L-45 | 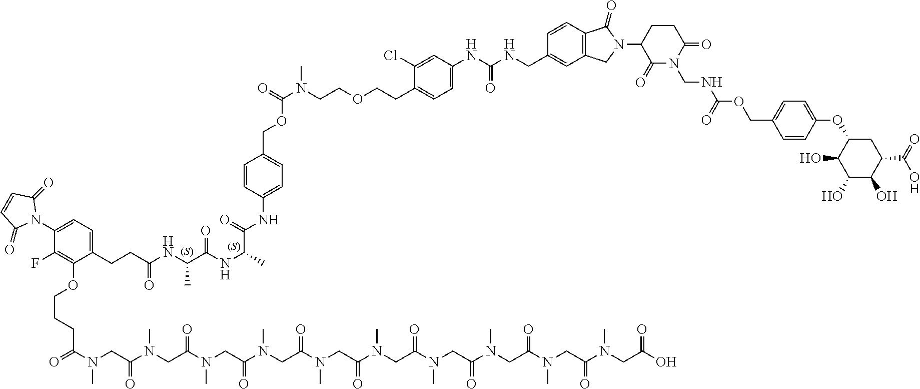 | 2232.7 |
| IG-L-46 | 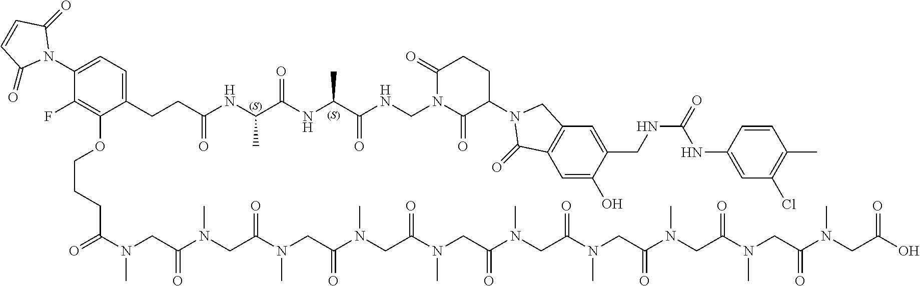 | 1686.2 |
| IG-L-47 | 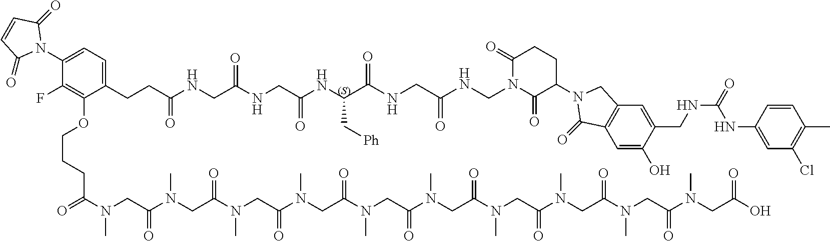 | 1862.4 |
| IG-L-48 | 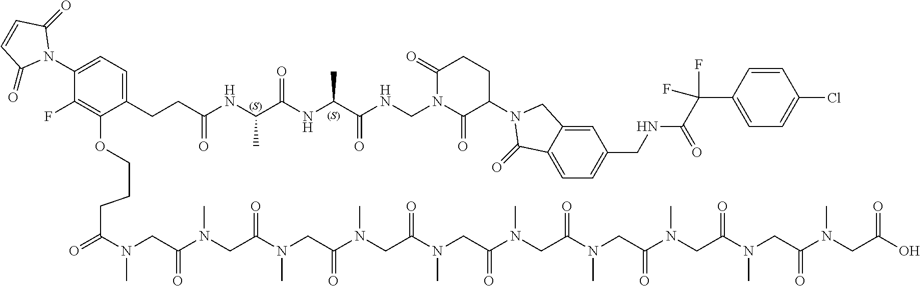 | 1691.1 |
| IG-L-49 | 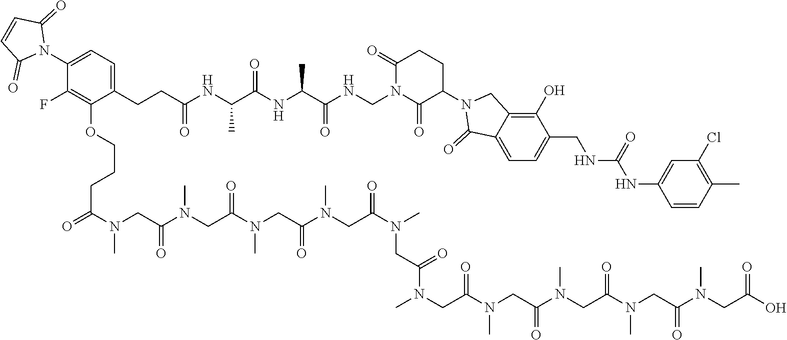 [SMILES: Cc1ccc(NC(=O)NCc2ccc3c(c2O)CN(C2CCC(=O)N(CNC(=O)[C@H](C)NC(=O)[C@H](C)NC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl] | 1686.2 |
| IG-L-50 | 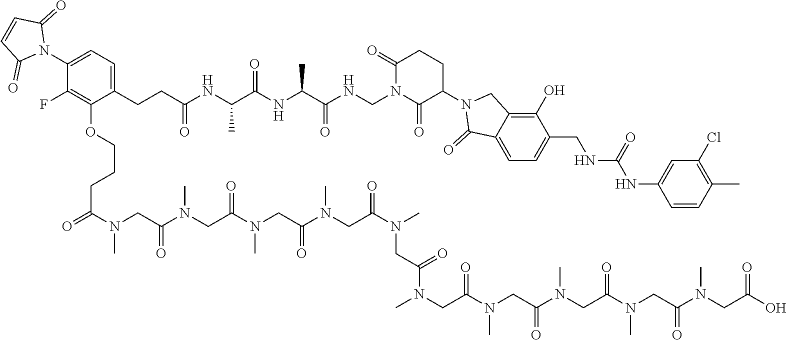 [SMILES: Cc1ccc(NC(=O)NCc2ccc3c(c2O)CN(C2CCC(=O)N(CNC(=O)[C@H](C)NC(=O)[C@H](C)NC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl] | 1862.4 |
| IG-L-51 | 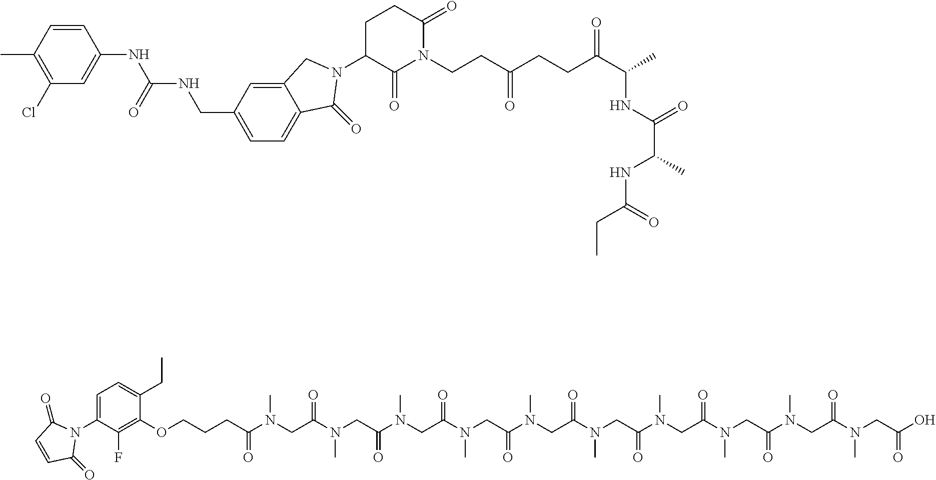 [SMILES: CCC(=O)N[C@@H](C)C(=O)N[C@@H](C)C(=O)CCC(=O)CCN1C(=O)CCC(N2Cc3cc(CNC(=O)Nc4ccc(C)c(Cl)c4)ccc3C2=O)C1=O ; CCc1ccc(N2C(=O)C=CC2=O)c(F)c1OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O] | 1727.2 |
| IG-L-52 | 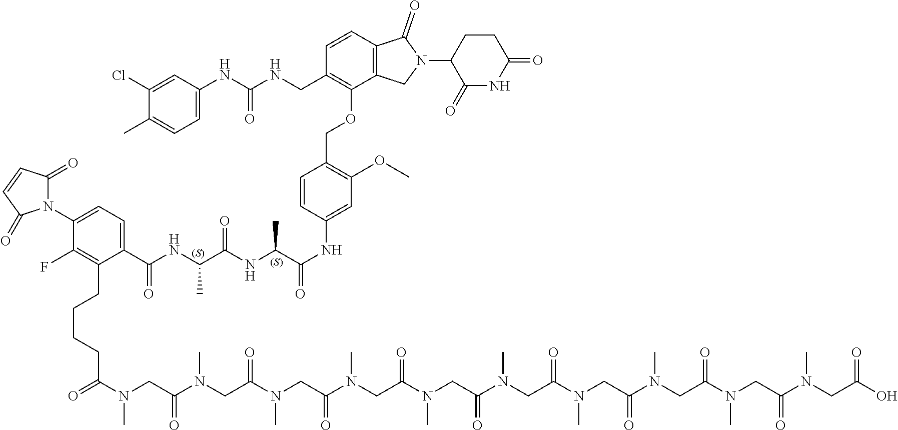 | 1792.3 |
| IG-L-53 | 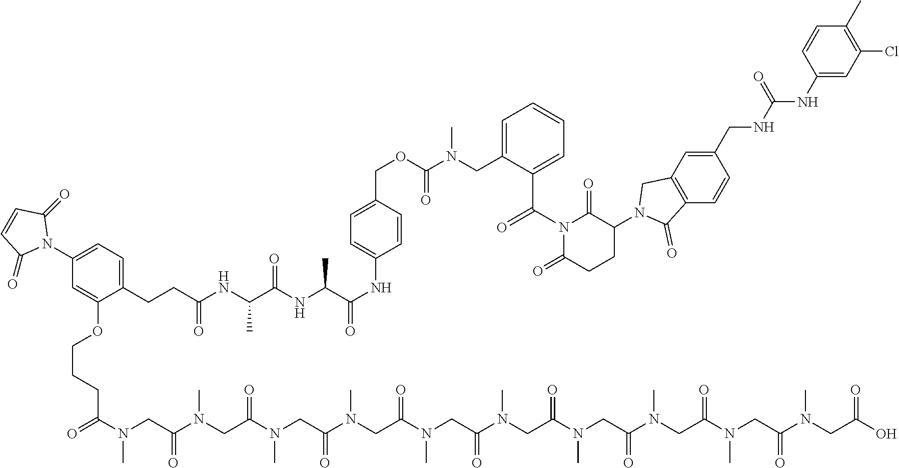 [SMILES: Cc1ccc(NC(=O)NCc2ccc3c(c2)CN(C2CCC(=O)N(C(=O)c4ccccc4CN(C)C(=O)OCc4ccc(NC(=O)[C@H](C)NC(=O)[C@H](C)NC(=O)CCc5ccc(N6C(=O)C=CC6=O)cc5OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc4)C2=O)C3=O)cc1Cl] | 1937.5 |
| IG-L-54 | 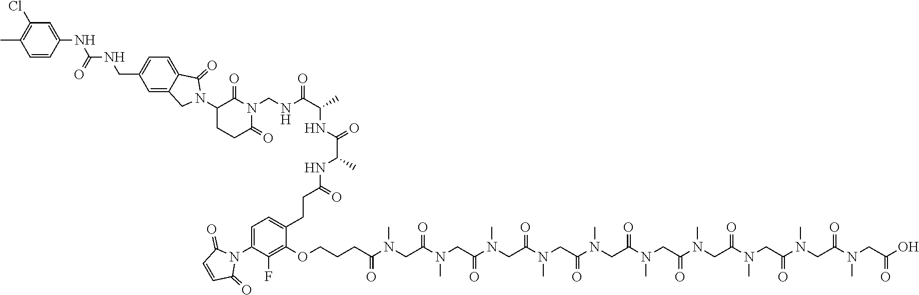 [SMILES: Cc1ccc(NC(=O)NCc2ccc3c(c2)CN(C2CCC(=O)N(CNC(=O)[C@H](C)NC(=O)[C@H](C)NC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl] | 1670.2 |
| IG-L-55 | 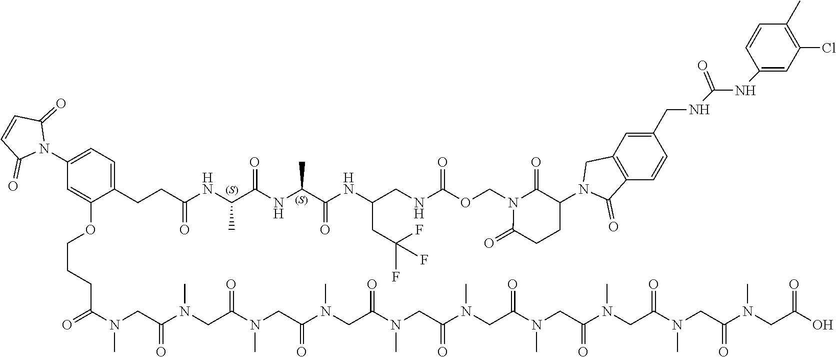 | 1821.3 |
| IG-L-56 | 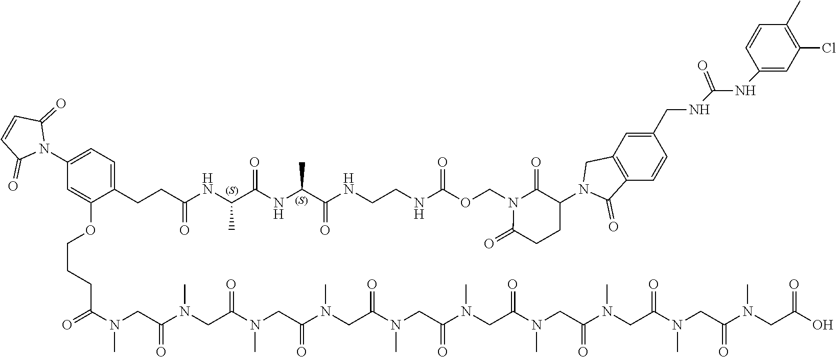 | 1739.3 |
| IG-L-57 | 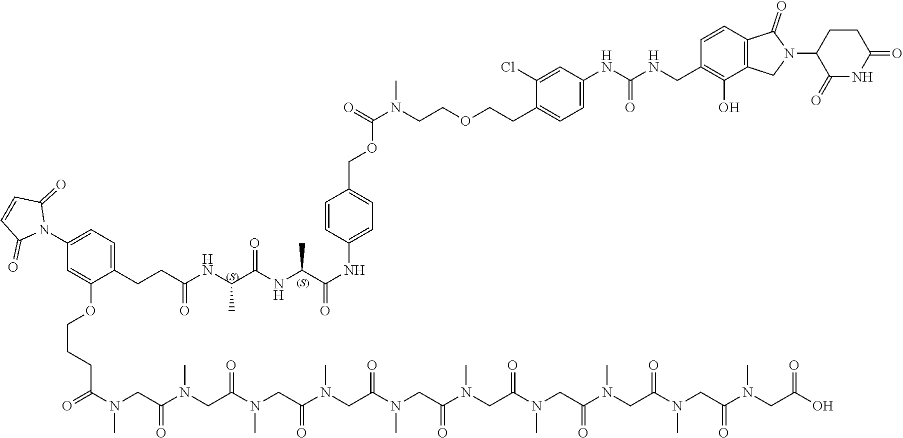 | 1893.4 |
| IG-L-58 | 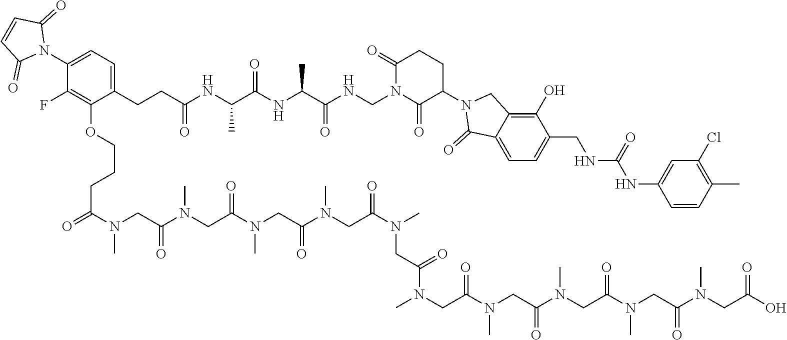 [SMILES: Cc1ccc(NC(=O)NCc2ccc3c(c2O)CN(C2CCC(=O)N(CNC(=O)[C@H](C)NC(=O)[C@H](C)NC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl] | 1828.4 |
| IG-L-59 | 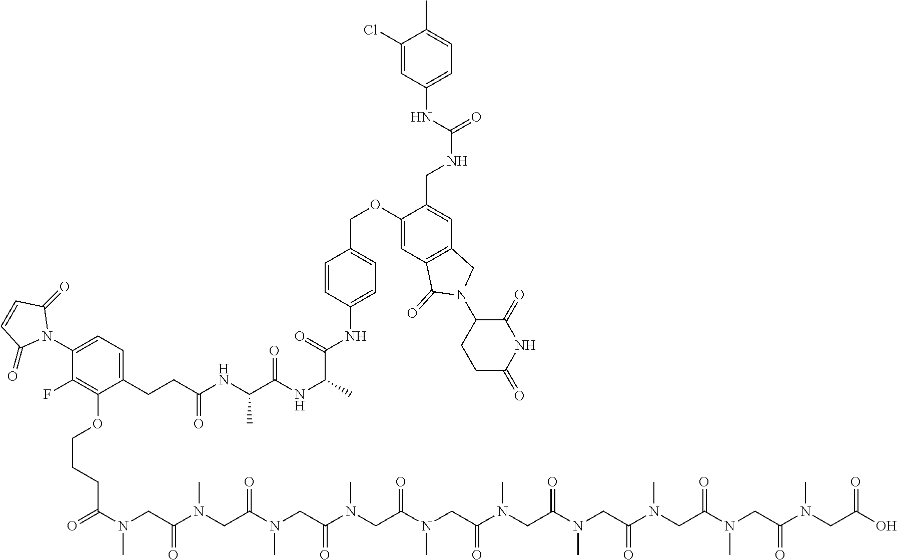 [SMILES: Cc1ccc(NC(=O)NCc2cc3c(cc2OCc2ccc(NC(=O)[C@H](C)NC(=O)[C@H](C)NC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc2)C(=O)N(C2CCC(=O)NC2=O)C3)cc1Cl] | 1744.3 |
| IG-L-60 | 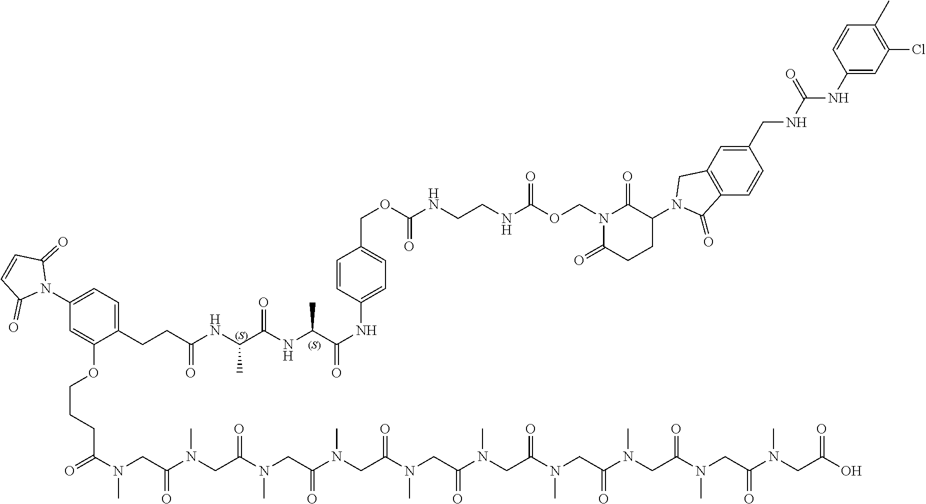 | 1888.4 |
| IG-L-61 | 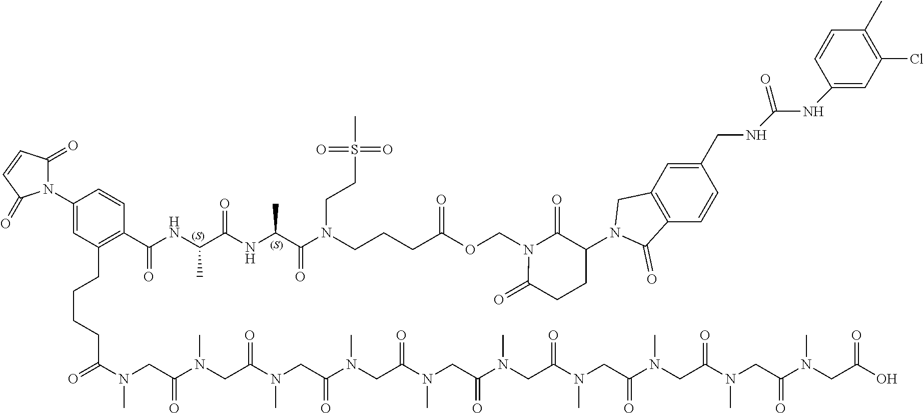 | 1845.4 |
| IG-L-62 | 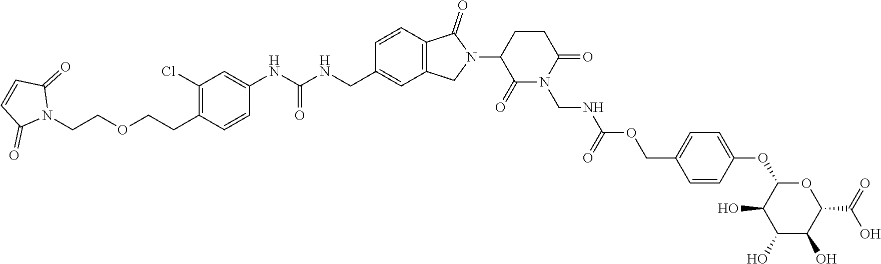 [SMILES: O=C(NCc1ccc2c(c1)CN(C1CCC(=O)N(CNC(=O)OCc3ccc(O[C@@H]4O[C@H](C(=O)O)[C@@H](O)[C@H](O)[C@H]4O)cc3)C1=O)C2=O)Nc1ccc(CCOCCN2C(=O)C=CC2=O)c(Cl)c1] | 949.3 |
| IG-L-63 | 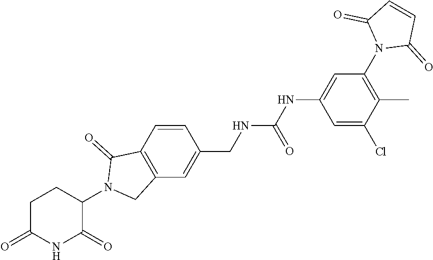 [SMILES: Cc1c(Cl)cc(NC(=O)NCc2ccc3c(c2)CN(C2CCC(=O)NC2=O)C3=O)cc1N1C(=O)C=CC1=O] | 535.9 |
| IG-L-64 | 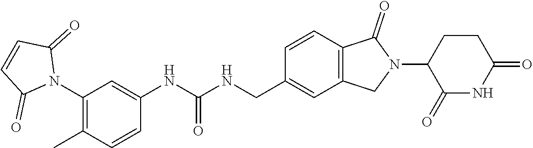 [SMILES: Cc1ccc(NC(=O)NCc2ccc3c(c2)CN(C2CCC(=O)NC2=O)C3=O)cc1N1C(=O)C=CC1=O] | 501.5 |
| IG-L-65 | 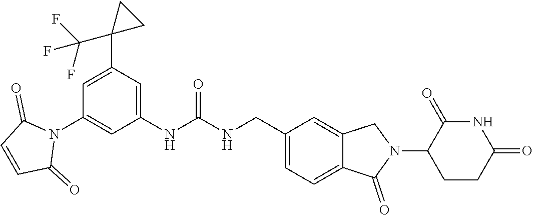 [SMILES: O=C1CCC(N2Cc3cc(CNC(=O)Nc4cc(N5C(=O)C=CC5=O)cc(C5(C(F)(F)F)CC5)c4)ccc3C2=O)C(=O)N1] | 595.5 |
| IG-L-66 | 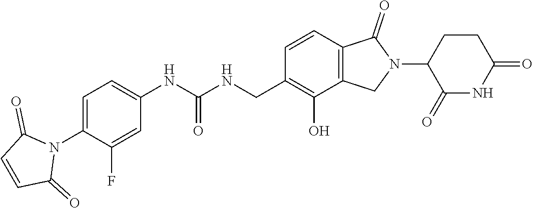 [SMILES: O=C1CCC(N2Cc3c(ccc(CNC(=O)Nc4ccc(N5C(=O)C=CC5=O)c(F)c4)c3O)C2=O)C(=O)N1] | 521.5 |
| IG-L-67 | 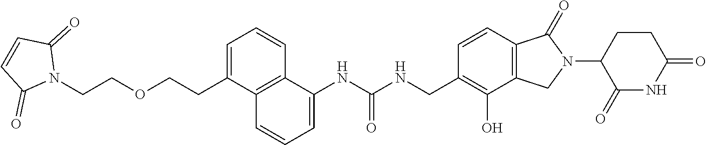 [SMILES: O=C1CCC(N2Cc3c(ccc(CNC(=O)Nc4cccc5c(CCOCCN6C(=O)C=CC6=O)cccc45)c3O)C2=O)C(=O)N1] | 625.4 |
| IG-L-68 | 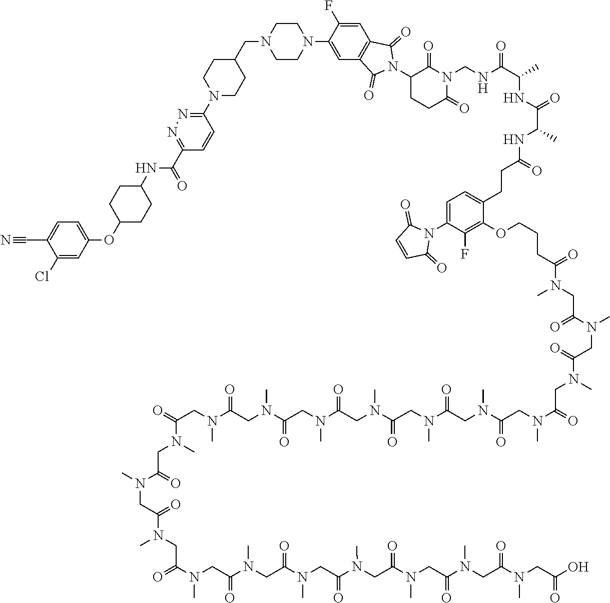 [SMILES: C[C@H](NC(=O)CCc1ccc(N2C(=O)C=CC2=O)c(F)c1OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C(=O)N[C@@H](C)C(=O)NCN1C(=O)CCC(N2C(=O)c3cc(F)c(N4CCN(CC5CCN(c6ccc(C(=O)NC7CCC(Oc8ccc(C#N)c(Cl)c8)CC7)nn6)CC5)CC4)cc3C2=O)C1=O] | 2752.4 |
| IG-L-69 | 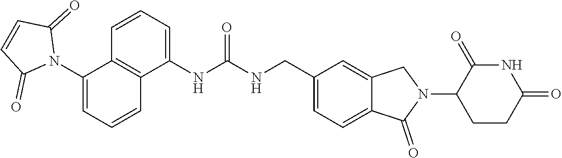 [SMILES: O=C1CCC(N2Cc3cc(CNC(=O)Nc4cccc5c(N6C(=O)C=CC6=O)cccc45)ccc3C2=O)C(=O)N1] | 537.5 |
| IG-L-70 | 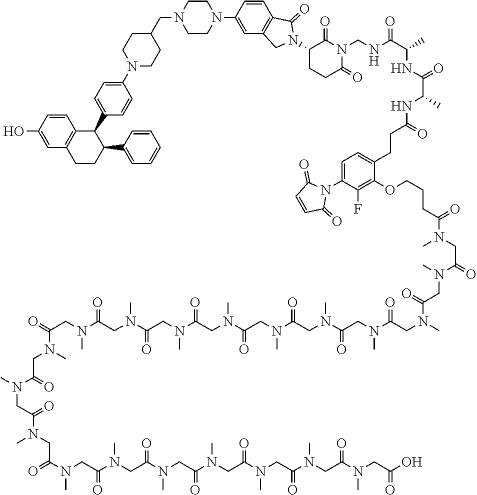 [SMILES: C[C@H](NC(=O)CCc1ccc(N2C(=O)C=CC2=O)c(F)c1OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C(=O)N[C@@H](C)C(=O)NCN1C(=O)CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C1=O] | 2664.0 |
| IG-L-71 | 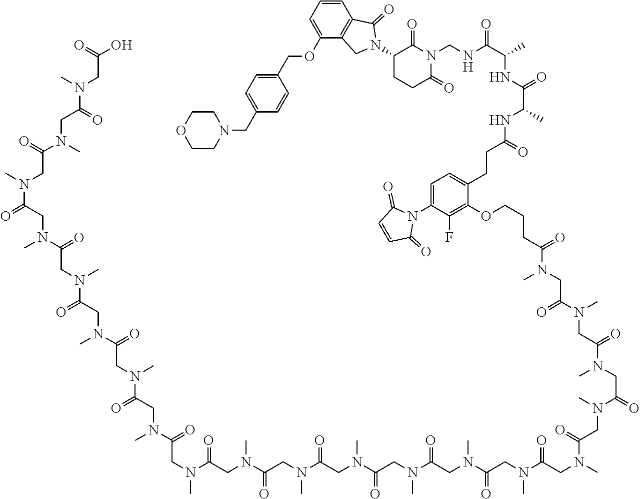 [SMILES: C[C@H](NC(=O)CCc1ccc(N2C(=O)C=CC2=O)c(F)c1OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C(=O)N[C@@H](C)C(=O)NCN1C(=O)CC[C@H](N2Cc3c(OCc4ccc(CN5CCOCC5)cc4)cccc3C2=O)C1=O] | 2389.6 |
| IG-L-72 | 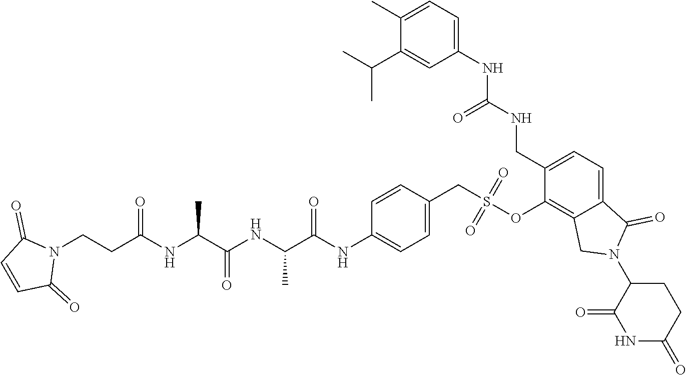 [SMILES: Cc1ccc(NC(=O)NCc2ccc3c(c2OS(=O)(=O)Cc2ccc(NC(=O)[C@H](C)NC(=O)[C@H](C)NC(=O)CCN4C(=O)C=CC4=O)cc2)CN(C2CCC(=O)NC2=O)C3=O)cc1C(C)C] | 927.0 |
| IG-L-73 | 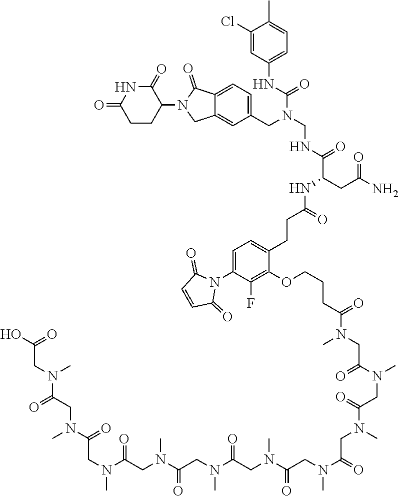 [SMILES: Cc1ccc(NC(=O)N(CNC(=O)[C@H](CC(N)=O)NC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2ccc3c(c2)CN(C2CCC(=O)NC2=O)C3=O)cc1Cl] | 1642.1 |
| IG-L-74 | 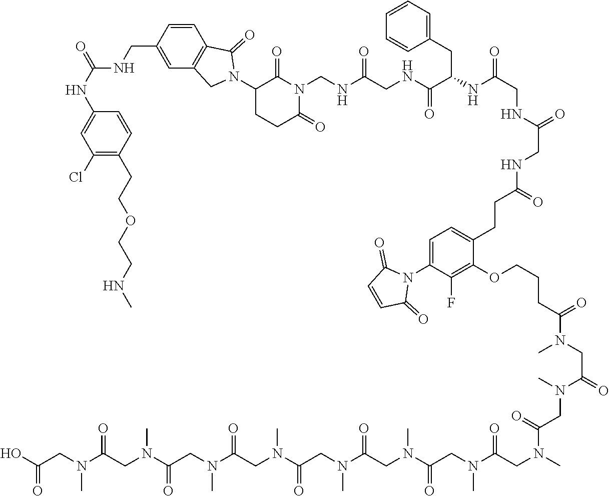 [SMILES: CNCCOCCc1ccc(NC(=O)NCc2ccc3c(c2)CN(C2CCC(=O)N(CNC(=O)CNC(=O)[C@H](Cc4ccccc4)NC(=O)CNC(=O)CNC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl] | 1933.5 |
| IG-L-75 | 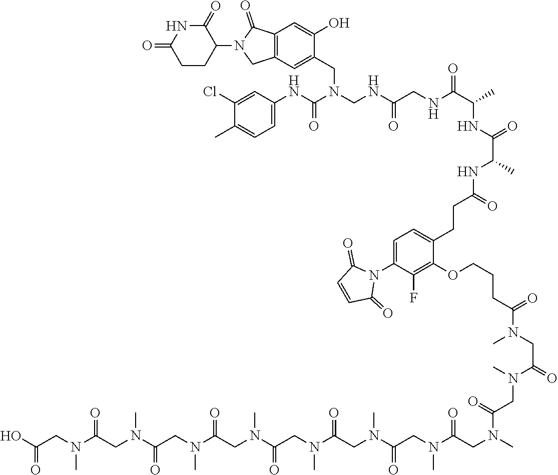 [SMILES: Cc1ccc(NC(=O)N(CNC(=O)CNC(=O)[C@H](C)NC(=O)[C@H](C)NC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2cc3c(cc2O)C(=O)N(C2CCC(=O)NC2=O)C3)cc1Cl] | 1743.2 |
| IG-L-76 | 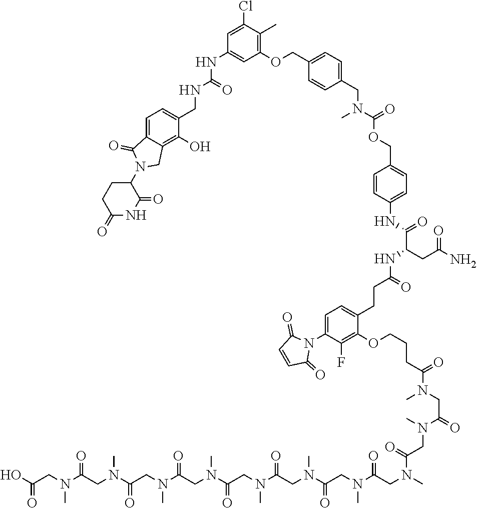 | 1927.4 |
| IG-L-77 | 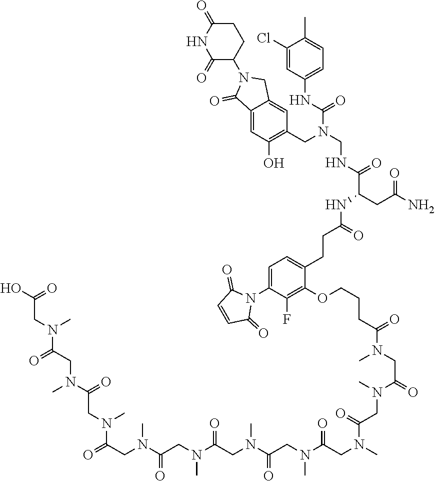 [SMILES: Cc1ccc(NC(=O)N(CNC(=O)[C@H](CC(N)=O)NC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2cc3c(cc2O)C(=O)N(C2CCC(=O)NC2=O)C3)cc1Cl] | 1658.1 |
| IG-L-78 | 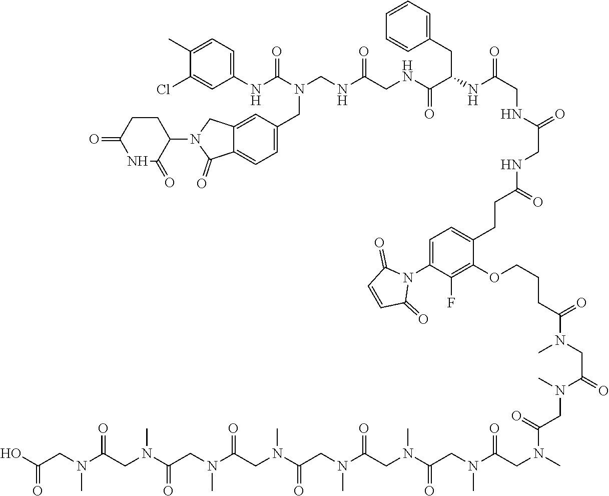 [SMILES: Cc1ccc(NC(=O)N(CNC(=O)CNC(=O)[C@H](Cc2ccccc2)NC(=O)CNC(=O)CNC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2ccc3c(c2)CN(C2CCC(=O)NC2=O)C3=O)cc1Cl] | 1846.3 |
| IG-L-79 | 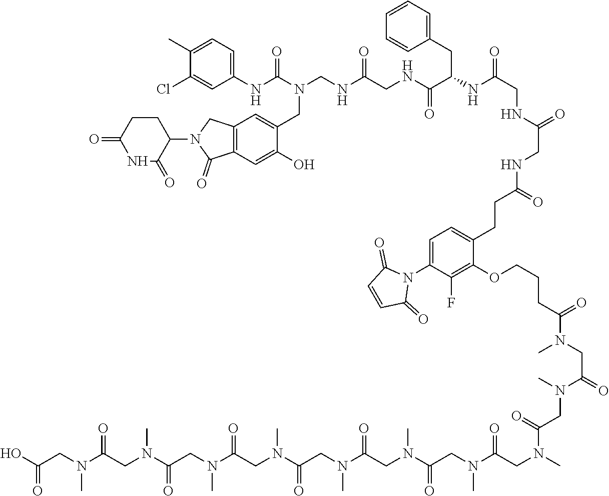 [SMILES: Cc1ccc(NC(=O)N(CNC(=O)CNC(=O)[C@H](Cc2ccccc2)NC(=O)CNC(=O)CNC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2cc3c(cc2O)C(=O)N(C2CCC(=O)NC2=O)C3)cc1Cl] | 1862.3 |
| IG-L-80 | 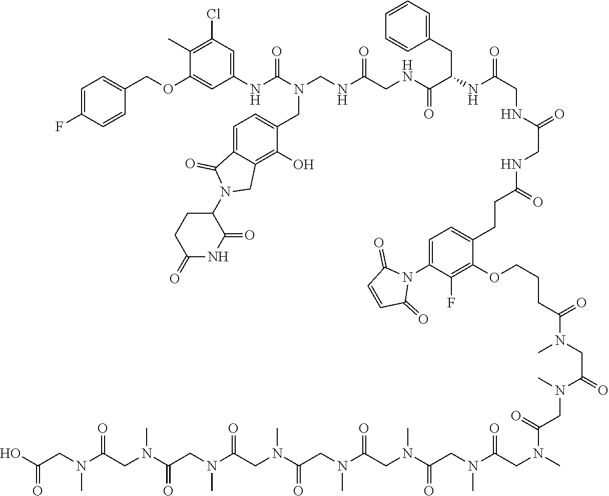 [SMILES: Cc1c(Cl)cc(NC(=O)N(CNC(=O)CNC(=O)[C@H](Cc2ccccc2)NC(=O)CNC(=O)CNC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2ccc3c(c2O)CN(C2CCC(=O)NC2=O)C3=O)cc1OCc1ccc(F)cc1] | 1986.5 |
| IG-L-81 | 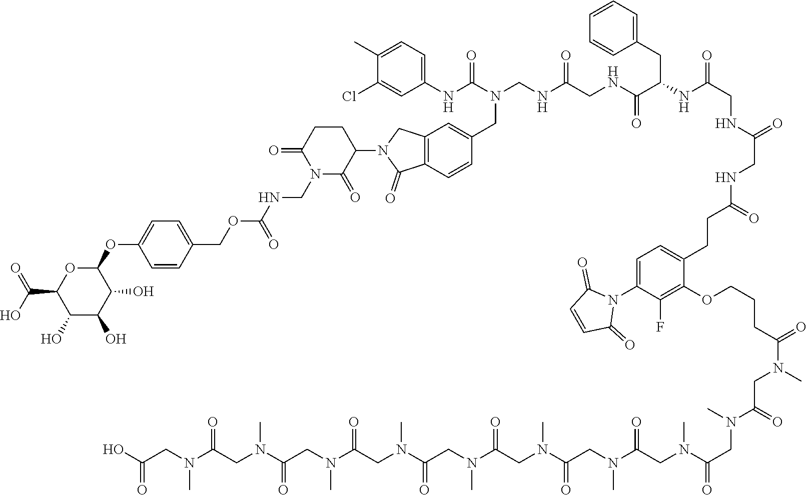 [SMILES: Cc1ccc(NC(=O)N(CNC(=O)CNC(=O)[C@H](Cc2ccccc2)NC(=O)CNC(=O)CNC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2ccc3c(c2)CN(C2CCC(=O)N(CNC(=O)OCc4ccc(O[C@@H]5O[C@H](C(=O)O)[C@@H](O)[C@H](O)[C@H]5O)cc4)C2=O)C3=O)cc1Cl] | 2201.6 |
| IG-L-82 | 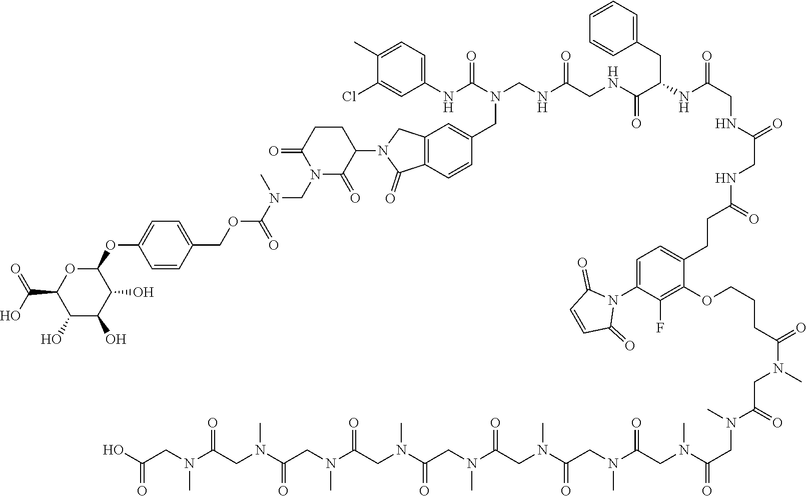 [SMILES: Cc1ccc(NC(=O)N(CNC(=O)CNC(=O)[C@H](Cc2ccccc2)NC(=O)CNC(=O)CNC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2ccc3c(c2)CN(C2CCC(=O)N(CN(C)C(=O)OCc4ccc(O[C@@H]5O[C@H](C(=O)O)[C@@H](O)[C@H](O)[C@H]5O)cc4)C2=O)C3=O)cc1Cl] | 2215.7 |

### Isoindolinone-Glutarimide Antibody Conjugates

The isoindolinone-glutarimide antibody conjugates (IGAC) of the invention induce target-specific protein degradation. Tumor targeting brings specificity to minimize off-target effects.

The isoindolinone-glutarimide antibody conjugates (IGAC) of the invention comprise an isoindolinone-glutarimide moiety covalently attached to an antibody by an antibody linker, wherein the antibody binds to a tumor-associated antigen or cell-surface receptor.

Exemplary embodiments of IGAC include Formula I:

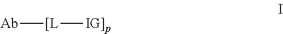

or a pharmaceutically acceptable salt thereof,
wherein:
Ab is the antibody;
L is the antibody linker;
IG is the isoindolinone-glutarimide moiety; and
p is an integer from 1 to 12.

An exemplary embodiment of the IGAC of Formula I includes wherein a phenolic oxygen of the isoindolinone-glutarimide moiety is attached to the antibody linker.

An exemplary embodiment of the IGAC of Formula I includes wherein the nitrogen of the glutarimide group is attached to the antibody linker.

An exemplary embodiment of the IGAC of Formula I includes wherein a solubilizing unit selected from polyglycine, polysarcosine, polyethyleneoxy (PEG), C₁-C₄₀ heteroalkyl, and a glycoside, or combinations thereof is attached to: (1) a phenolic oxygen, or (2) the nitrogen of the glutarimide group of the isoindolinone-glutarimide moiety.

An exemplary embodiment of the IGAC of Formula I includes wherein the terminus of the solubilizing unit is a group selected from an amino acid, amino, hydroxyl, hydrogen, carboxylic acid, glycerol, or a sugar such as pentaerythritol, maltitol, sorbitol, xylitol, erythritol, isomalt, or combinations thereof.

An exemplary embodiment of the IGAC is selected from Formulae Ia and Ilb:

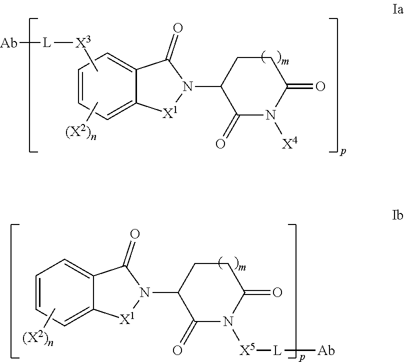

wherein:
m is 0, 1 or 2;
X¹ is selected from the group consisting of CH₂, C(═O) and N═N;
X² is independently selected from the group consisting of F, Cl, Br, I, —CN, C₁-C₁₂ alkyl, C₂-C₁₂ alkenyl, C₂-C₁₂ alkynyl, C₁-C₁₂ heteroalkyl, —(C₁-C₁₂ heteroalkyldiyl)-(C₆-C₂₀ aryldiyl), —(C₁-C₁₂ heteroalkyldiyl)-(C₆-C₂₀ aryldiyl)-(C₁-C₁₂ heteroalkyl), —(C₁-C₁₂ heteroalkyldiyl)-(C₆-C₂₀ aryl), —(C₁-C₁₂ heteroalkyldiyl)-(C₆-C₂₀ aryldiyl)-(C₁-C₁₂ heteroalkyl), (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C20 heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, —OR^{a}, —OC(═O)R^{a}, —SR^{a}, —S(O)R^{a}, —S(O)₂R^{a}, —S(O)₂NR^{a}, and —S(O)₃H;
R^{a} is independently selected from H, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
R^{b} is independently selected from H, OH, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
n is 0, 1, 2, 3, or 4;
X³ is selected from the group consisting of a bond, 0, C₁-C₁₂ alkyldiyl, C₂-C₁₂ alkenyldiyl, C₂-C₁₂ alkynyldiyl, C₁-C₁₂ heteroalkyldiyl, —(C₁-C₆ alkyldiyl)-(C₆-C₂₀ aryldiyl)-, —(C₁-C₆ alkyldiyl)NR^{a}—, —(C₁-C₆ alkyldiyl)O—, —(C₁-C₆ alkyldiyl)-(C₃-C₂₀ carbocyclyldiyl)-, —(C₁-C₆ alkyldiyl)-(C₂-C₂₀ heterocyclyldiyl)-, —(C₁-C₆ alkyldiyl)-(C₁-C₂₀ heteroaryldiyl)-, —(C₁-C₁₂ heteroalkyldiyl)-(C₆-C₂₀ aryldiyl)-, —(C₁-C₁₂ heteroalkyldiyl)-(C₆-C₂₀ aryldiyl)-(C₁-C₁₂ heteroalkyldiyl)-, —(C₁-C₁₂ heteroalkyldiyl)-(C₆-C₂₀ aryldiyl)-(C₁-C₁₂ heteroalkyldiyl)-(C₆-C₂₀ aryldiyl)-(C₁-C₁₂ heteroalkyldiyl)-, —(C₁-C₁₂ heteroalkyldiyl)-(C₆-C₂₀ aryl)-, C₆-C₂₀ aryldiyl, C₃-C₂₀ carbocyclyldiyl, C₂-C₂₀ heterocyclyldiyl, and C₁-C₂₀ heteroaryldiyl;
X⁴ is selected from the group consisting of H, C₁-C₁₂ heteroalkyl, —(C₁-C₁₂ heteroalkyldiyl)-(C₆-C₂₀ aryl), —(C₁-C₁₂ heteroalkyldiyl)-(C₆-C₂₀ aryldiyl)-(C₁-C₁₂ heteroalkyl);
X⁵ is selected from the group consisting of a bond, C₁-C₁₂ alkyldiyl, C₂-C₁₂ alkenyldiyl, C₂-C₁₂ alkynyldiyl, C₁-C₁₂ heteroalkyldiyl, —(C₁-C₆ alkyldiyl)-(C₆-C₂₀ aryldiyl), —(C₁-C₆ alkyldiyl)-(C₆-C₂₀ aryldiyl)-O—(C₂-C₂₀ heterocyclyl), —(C₁-C₆ alkyldiyl)NR^{a}—, —(C₁-C₆ alkyldiyl)O—, —(C₁-C₆ alkyldiyl)-(C3-C₂₀ carbocyclyldiyl), —C₁-C₆ alkyldiyl)-(C₂-C₂₀ heterocyclyldiyl), —(C₁-C₆ alkyldiyl)-(C₁-C₂₀ heteroaryldiyl), —(C₁-C₁₂ heteroalkyldiyl)-(C₆-C₂₀ aryldiyl), —(C₁-C₁₂ heteroalkyldiyl)-(C₆-C₂₀ aryldiyl)-(C₁-C₁₂ heteroalkyldiyl), C₆-C₂₀ aryldiyl, C₃-C₂₀ carbocyclyldiyl, C₂-C₂₀ heterocyclyldiyl, and C₁-C₂₀ heteroaryldiyl; and
L is the antibody linker;
wherein each alkyl, alkyldiyl, alkenyl, alkenyldiyl, alkynyl, alkynyldiyl, heteroalkyl, heteroalkyldiyl, aryl, aryldiyl, carbocyclyl, carbocyclyldiyl, heterocyclyl, heterocyclyldiyl, heteroaryl, and heteroaryldiyl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of the IGAC of Formula I includes wherein the antibody linker is covalently attached to a cysteine amino acid of the antibody.

An exemplary embodiment of the IGAC of Formula I includes wherein the antibody is a cysteine-engineered antibody.

An exemplary embodiment of the IGAC of Formulae Ia and Ib includes wherein m is 0.

An exemplary embodiment of the IGAC of Formulae Ia and Ib includes wherein m is 1.

An exemplary embodiment of the IGAC of Formulae Ia and Ib includes wherein m is 2.

An exemplary embodiment of the IGAC of Formulae Ia and Ib includes wherein X¹ is CH₂.

An exemplary embodiment of the IGAC of Formulae Ia and Ib includes wherein X¹ is C(═O).

An exemplary embodiment of the IGAC of Formulae Ia and Ib includes wherein X¹ is N═N.

An exemplary embodiment of the IGAC of Formulae Ia and Ib includes wherein X² comprises a —NHC(═O)NH— group.

An exemplary embodiment of the IGAC of Formulae Ia and Ib includes wherein X² is selected from the group consisting of C₁-C₁₂ heteroalkyl, —(C₁-C₁₂ heteroalkyldiyl)-(C₆-C₂₀ aryl), and —(C₁-C₁₂ heteroalkyldiyl)-(C₆-C₂₀ aryldiyl)-(C₁-C₁₂ heteroalkyl).

An exemplary embodiment of the IGAC of Formulae Ia and Ib includes wherein X² is —(C₁-C₁₂ heteroalkyldiyl)-(C₆-C₂₀ aryl).

An exemplary embodiment of the IGAC of Formulae Ia and Ib includes wherein X² is —CH₂NHC(═O)NH—(C₆-C₂₀ aryl).

An exemplary embodiment of the IGAC of Formulae Ia and Ib includes wherein X² is:

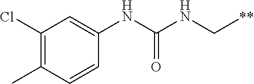

wherein:
** indicates the point of attachment to the isoindolinone-glutarimide moiety.

An exemplary embodiment of the IGAC of Formulae Ia includes wherein X³ is O.

An exemplary embodiment of the IGAC of Formulae Ja b includes wherein X³ comprises a —NHC(═O)NH— group.

An exemplary embodiment of the IGAC of Formulae Ia includes wherein X³ is —(C₁-C₁₂ heteroalkyldiyl)-(C₆-C₂₀ aryldiyl)-(C₁-C₁₂ heteroalkyldiyl).

An exemplary embodiment of the IGAC of Formulae Ia includes wherein X³ is —CH₂NHC(═O)NH—(C₆-C₂₀ aryldiyl)-(C₁-C₁₂ heteroalkyldiyl).

An exemplary embodiment of the IGAC of Formulae Ia includes wherein X³ is selected from —(C₁-C₁₂ heteroalkyldiyl)-(C₆-C₂₀ aryldiyl)-O—, —(C₁-C₁₂ heteroalkyldiyl)-(C₆-C₂₀ aryldiyl)-N(R^{a})—, and —(C₁-C₁₂ heteroalkyldiyl)-(C₆-C₂₀ aryldiyl)-(C₁-C₁₂ heteroalkyldiyl).

An exemplary embodiment of the IGAC of Formulae Ia includes wherein X³ is —CH₂CH₂CH₂NHC(═O)NH—(C₆-C₂₀ aryldiyl)-O—.

An exemplary embodiment of the IGAC of Formulae Ia includes wherein X³ is selected from the structures:

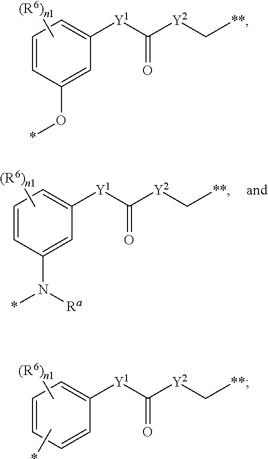

wherein:
* indicates the point of attachment to L;
** indicates the point of attachment to the phenyl glutarimide moiety;
R⁶ is independently selected from F, Cl, Br, I, —CN, OH, —O—(C₁-C₁₂ alkyl), C₁-C₁₂ alkyl, C₂-C₁₂ alkenyl, C₂-C₁₂ alkynyl, C₁-C₁₂ heteroalkyl, C₆-C₂₀ aryl, C₃-C₂₀ carbocyclyl, C₂-C₂₀ heterocyclyl, and C₁-C₂₀ heteroaryl;
n1 is 1, 2, 3, or 4;
Y¹ is selected from CF₂ and NH; and
Y² is selected from NH, O, and CH₂.

An exemplary embodiment of the IGAC of Formulae Ia includes wherein X³ is selected from:

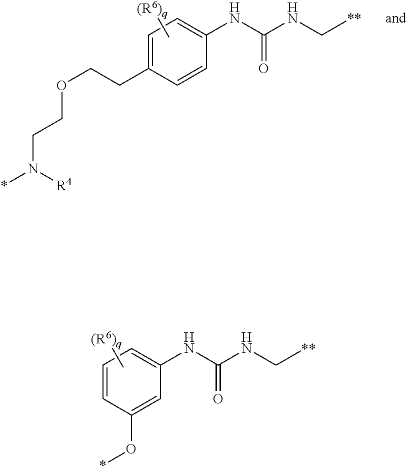

wherein:
* indicates the point of attachment to L;
** indicates the point of attachment to the isoindolinone-glutarimide moiety;
R⁴ is selected from H and C₁-C₁₂ alkyl;
R⁶ is independently selected from F, Cl, Br, I, —CN, OH, C₁-C₁₂ alkyl, C₂-C₁₂ alkenyl, C₂-C₁₂ alkynyl, and C₁-C₁₂ heteroalkyl; and
q is selected from 0, 1, 2, 3, and 4.

An exemplary embodiment of the IGAC of Formulae Ia includes wherein X³ is —(C₁-C₁₂ heteroalkyldiyl)-(C₆-C₂₀ aryl).

An exemplary embodiment of the IGAC of Formulae Ia includes wherein X³ is selected from:

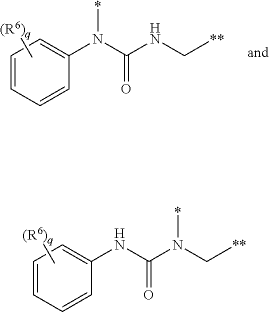

wherein:
* indicates the point of attachment to L;
** indicates the point of attachment to the isoindolinone-glutarimide moiety;
R⁶ is independently selected from F, Cl, Br, I, —CN, OH, C₁-C₁₂ alkyl, C₂-C₁₂ alkenyl, C₂-C₁₂ alkynyl, and C₁-C₁₂ heteroalkyl; and
q is selected from 0, 1, 2, 3, and 4.

An exemplary embodiment of the IGAC of Formula I includes Formula Ia:

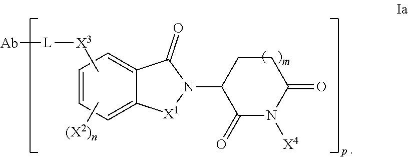

An exemplary embodiment of the IGAC of Formula Ia includes wherein X² is —OH and n is 1.

An exemplary embodiment of the IGAC of Formula Ia includes the structures:

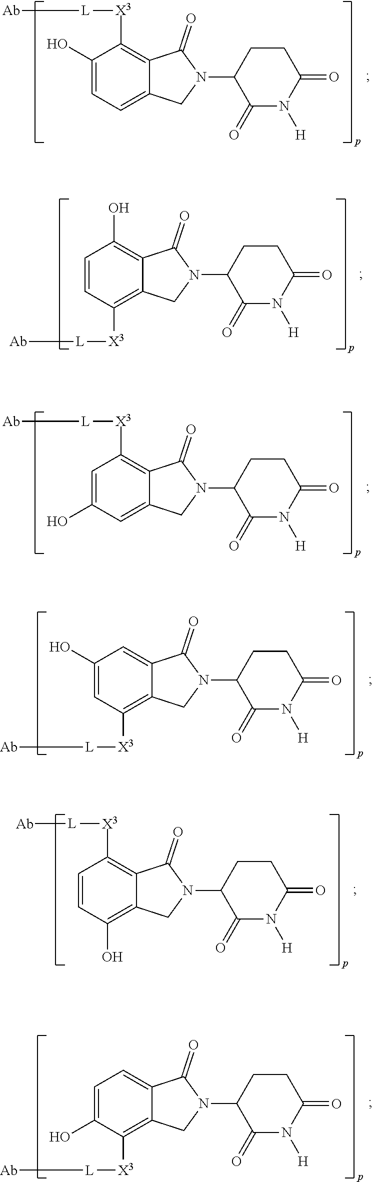

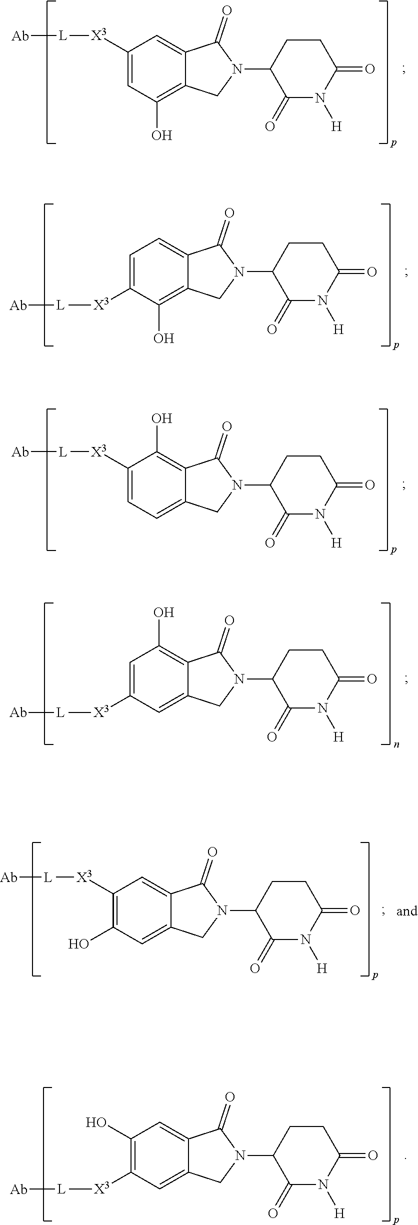

An exemplary embodiment of the IGAC of Formula Ia includes wherein X⁴ is H.

An exemplary embodiment of the IGAC of Formula Ia includes wherein X⁴ is selected from the group consisting of C₁-C₁₂ heteroalkyl, —(C₁-C₁₂ heteroalkyldiyl)-(C₆-C₂₀ aryl), —(C₁-C₁₂ heteroalkyldiyl)-(C₆-C₂₀ aryldiyl)-(C₁-C₁₂ heteroalkyl), and —(C₁-C₁₂ heteroalkyldiyl)-(C₆-C₂₀ aryldiyl)-O—(C2-C₂₀ heterocyclyl).

An exemplary embodiment of the IGAC of Formula Ia includes wherein X⁴ is —(C₁-C₁₂ heteroalkyldiyl)-(C₆-C₂₀ aryldiyl)-O—(C₂-C₂₀ heterocyclyl), and C₂-C₂₀ heterocyclyl is a glucuronide.

An exemplary embodiment of the IGAC of Formula Ia includes wherein X⁴ of formula Ia is selected from the formulae:

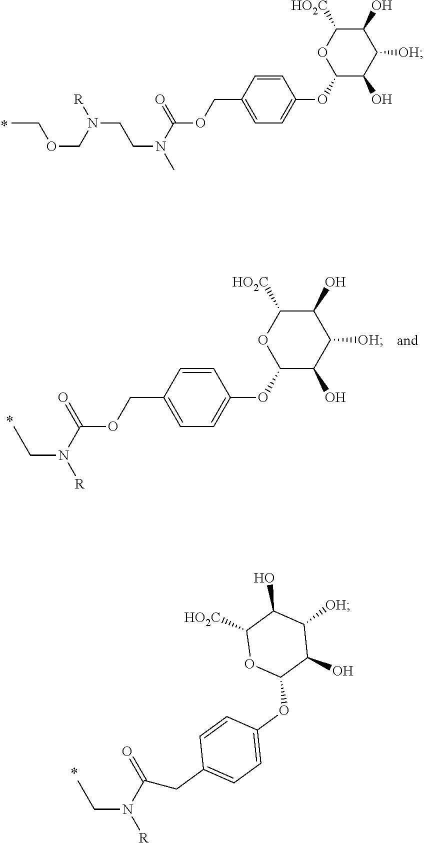

wherein R is selected from H, C₁-C₆ alkyl, and O—(C₁-C₆ alkyl); and * indicates the point of attachment to IG.

An exemplary embodiment of the IGAC of Formula I includes Formula Ib:

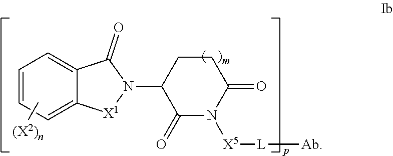

An exemplary embodiment of the IGAC of Formula Ib includes wherein n is 2, 3 or 4 and one of X² is —OH.

An exemplary embodiment of the IGAC of Formula Ib includes the structures:

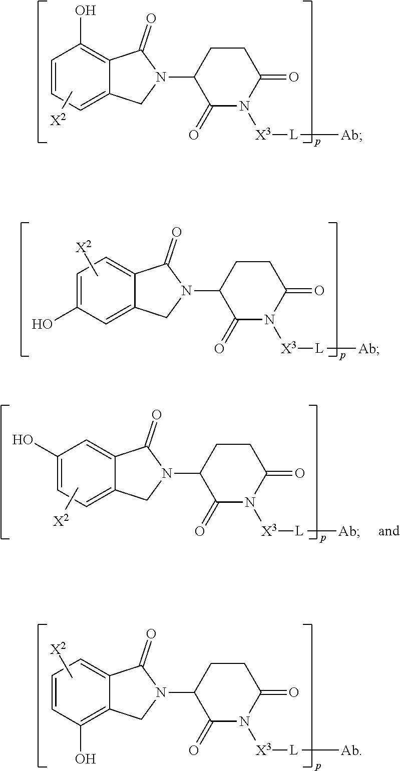

An exemplary embodiment of the IGAC of Formula Ib includes wherein X⁵ is selected from —(C₁-C₁₂ heteroalkyldiyl)-(C₆-C₂₀ aryldiyl) and —(C₁-C₆ alkyldiyl)-(C₆-C₂₀ aryldiyl).

Exemplary embodiments of the IGAC of Formula I includes Formulae Ic-g:

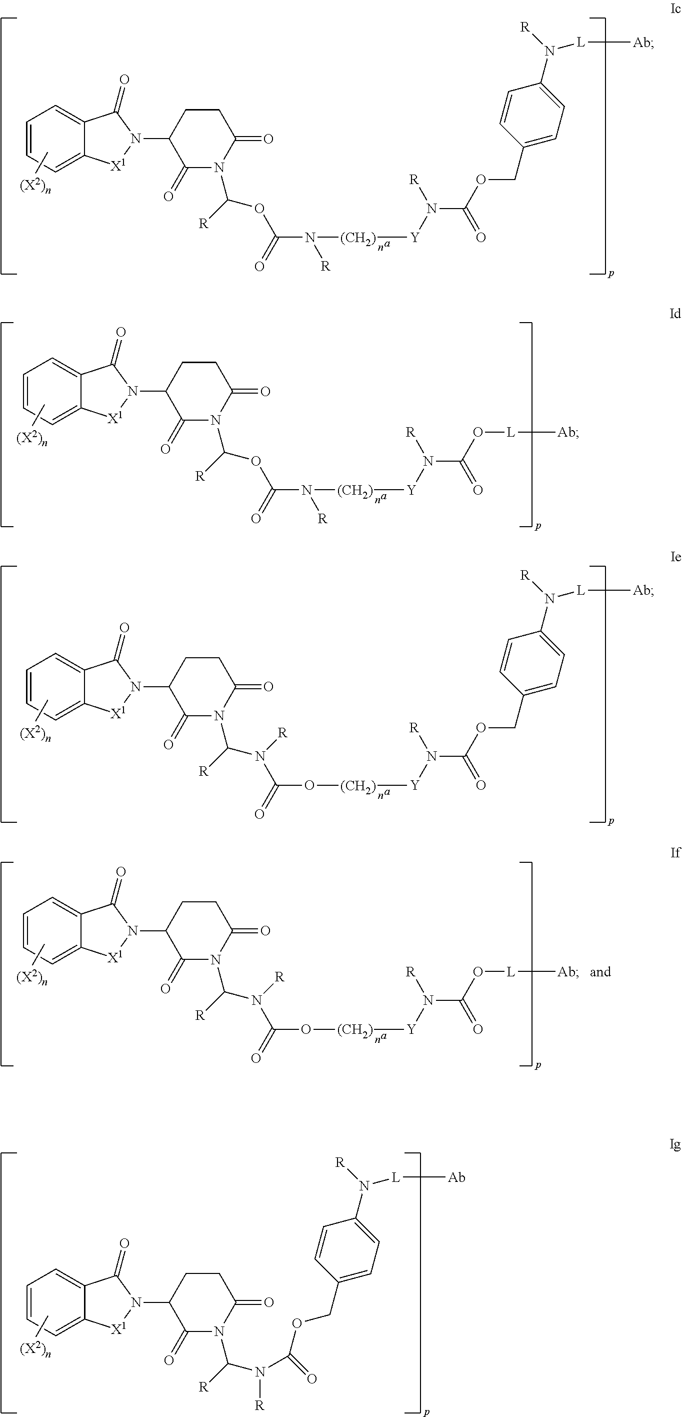

where R is independently selected from H, C₁-C₆ alkyl, and O—(C₁-C₆ alkyl); n^{a} is an integer from 1 to 5; and Y is CH₂ or O.

An exemplary embodiment of the IGAC of Formulas Ic-g includes wherein R is —CH₃ and n^{a} is 1.

An exemplary embodiment of the IGAC of Formulas Ic-g includes X² is —OH and n is 1.

Exemplary embodiments of the IGAC of Formula I include Formulae Ih and Ii:

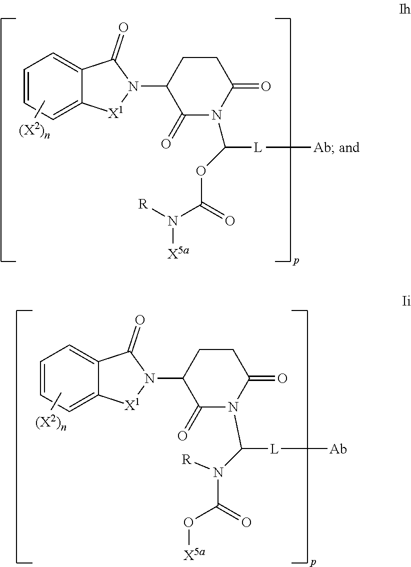

wherein R is selected from H, C₁-C₆ alkyl, and O—(C₁-C₆ alkyl); and X^{5a} is —(C₁-C₆ alkyldiyl)-(C₆-C₂₀ aryldiyl)-O—(C₂-C₂₀ heterocyclyl).

An exemplary embodiment of the IGAC of Formulas Ih and Ii includes wherein C₂-C₂₀ heterocyclyl of X^{5a} is a glucuronide.

An exemplary embodiment of the IGAC of Formulas Ih and Ii includes wherein X^{5a} is:

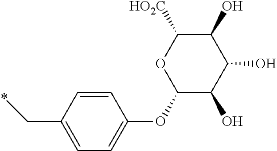 [SMILES: *Cc1ccc(O[C@@H]2O[C@H](C(=O)O)[C@@H](O)[C@H](O)[C@H]2O)cc1]

wherein * indicates the point of attachment.

An exemplary embodiment of the IGAC of Formulas Ih and Ii includes wherein L has the structure:

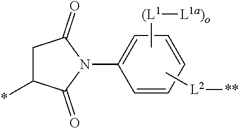

wherein:
L¹ is selected from a bond, C₁-C₁₂ alkyldiyl, and C₁-C₄₀ heteroalkyldiyl;
L^{1a} is selected from an amino acid, amino, hydroxyl, halide, hydrogen, carboxylic acid, glycerol, or a sugar such as pentaerythritol, maltitol, sorbitol, xylitol, erythritol, isomalt, or combinations thereof;
o is 0, 1, or 2;
L² is selected from a bond, C₁-C₁₂ alkyldiyl, and C₁-C₄₀ heteroalkyldiyl;
* indicates the point of attachment to a cysteine thiol of Ab; and
** indicates the point of attachment to the isoindolinone-glutarimide moiety.

Exemplary embodiments of the IGAC of Formula I include Formulae Ij-m:

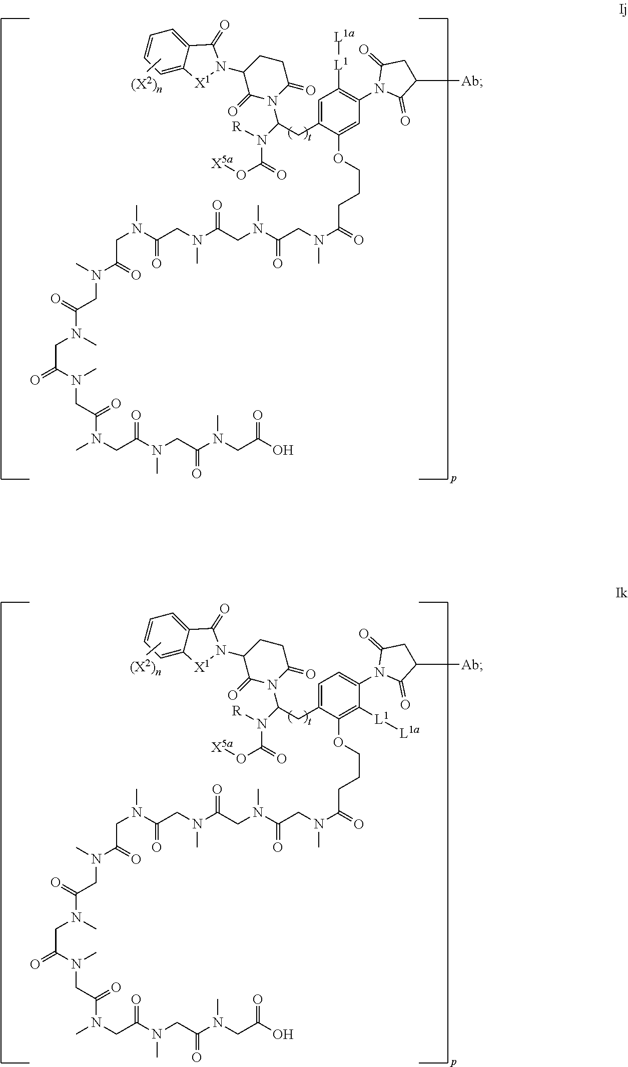

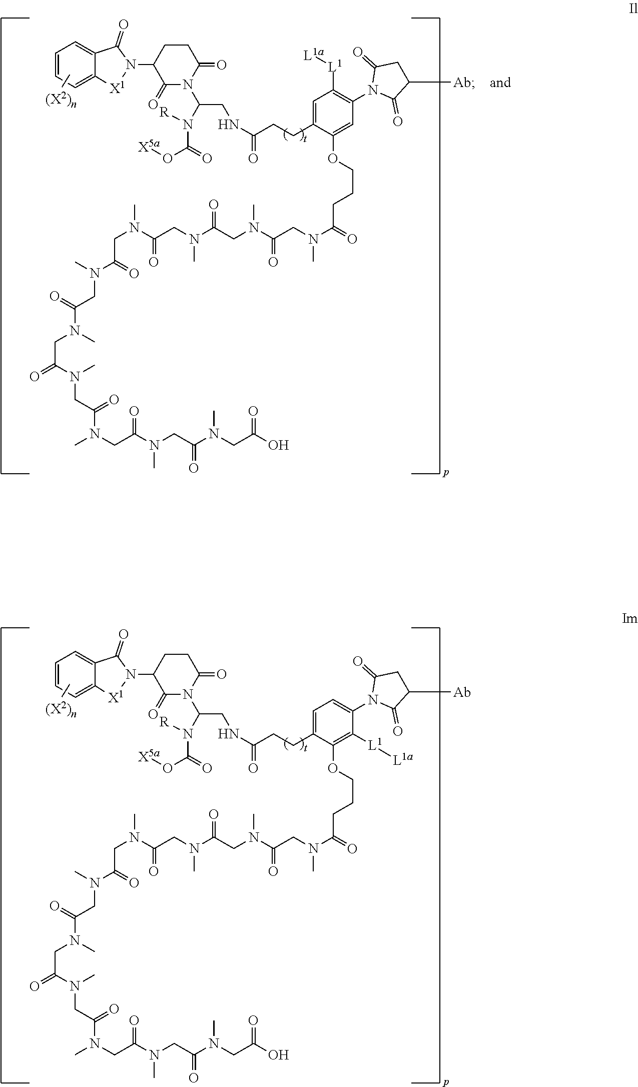

where t is selected from 0, 1, 2, 3, and 4.

An exemplary embodiment of the IGAC of Formulae Ij-m includes wherein L¹ is a bond and L¹a is F.

An exemplary embodiment of the IGAC of Formula I includes wherein the antibody linker comprises an immolating group.

An exemplary embodiment of the IGAC of Formula I includes wherein the antibody linker comprises a peptide unit.

An exemplary embodiment of the IGAC of Formula I includes wherein L has the formula:

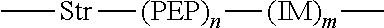

wherein:
Str is a stretcher unit covalently attached to the antibody;
PEP is a protease-cleavable, peptide unit covalently attached to Str and IM or IG;
IM is an immolative unit covalently attached to IG;
n is 0 or 1; and m is 0 or 1.

An exemplary embodiment of the IGAC of Formula I includes wherein IG is attached to L by Str.

An exemplary embodiment of the IGAC of Formula I includes wherein IG is attached to L by PEP.

An exemplary embodiment of the IGAC of Formula I includes wherein IG is attached to L by IM.

An exemplary embodiment of the IGAC of Formula I includes wherein Str is a branched linker covalently attached to: (i) the antibody; and (ii) a solubilizing unit comprising a group selected from polyglycine, polysarcosine, polyethyleneoxy (PEG), C₁-C₄₀ heteroalkyl, and a glycoside, or combinations thereof, wherein the terminus of the solubilizing unit is a group selected from an amino acid, amino, hydroxyl, hydrogen, carboxylic acid, glycerol, or a sugar such as pentaerythritol, maltitol, sorbitol, xylitol, erythritol, isomalt, or combinations thereof.

An exemplary embodiment of the IGAC of Formula I includes wherein the solubilizing unit and the terminus of the solubilizing unit covalently attached to Str are selected from the structures:

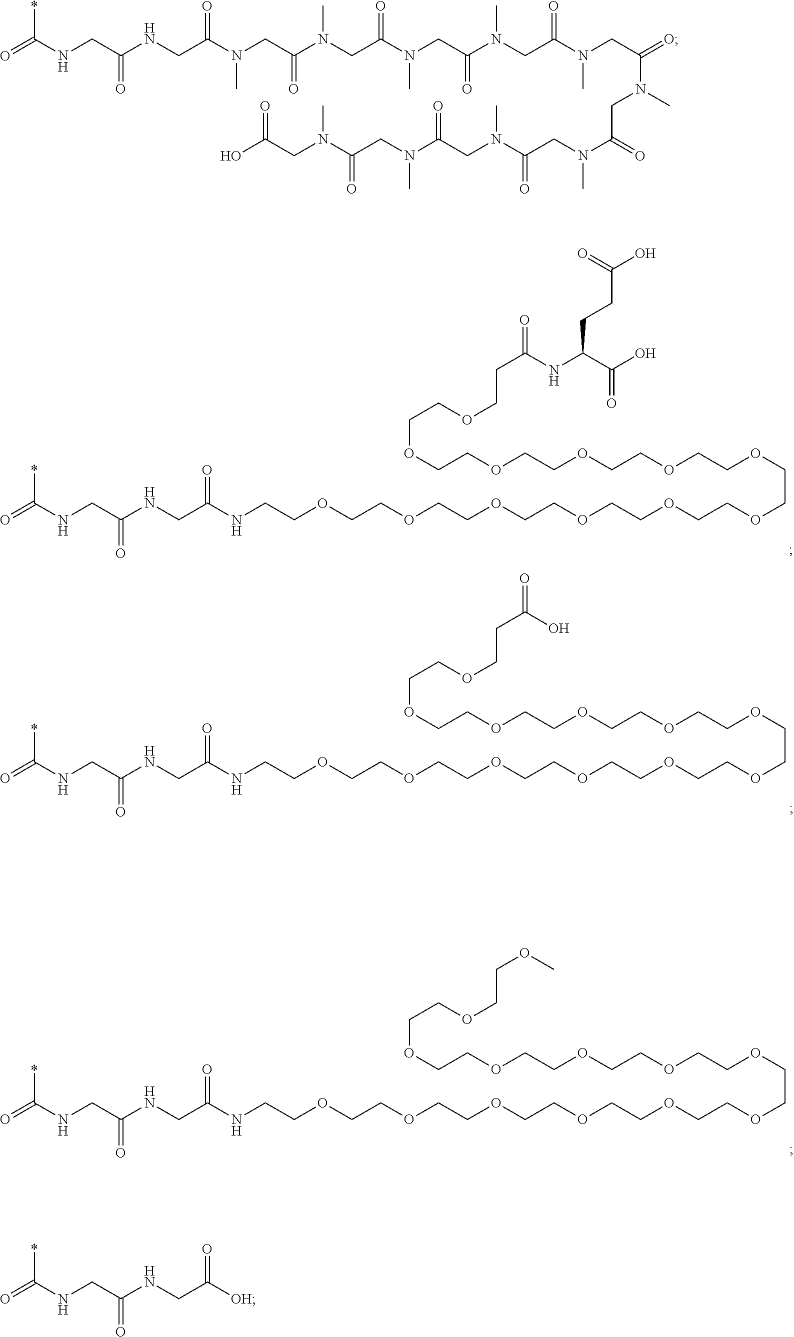 [SMILES: *C(=O)NCC(=O)NCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O ; *C(=O)NCC(=O)NCC(=O)NCCOCCOCCOCCOCCOCCOCCOCCOCCOCCOCCOCCOCCC(=O)N[C@@H](CCC(=O)O)C(=O)O ; *C(=O)NCC(=O)NCC(=O)NCCOCCOCCOCCOCCOCCOCCOCCOCCOCCOCCOCCOCCC(=O)O ; *C(=O)NCC(=O)NCC(=O)NCCOCCOCCOCCOCCOCCOCCOCCOCCOCCOCCOCCOCCOC ; *C(=O)NCC(=O)NCC(=O)O]

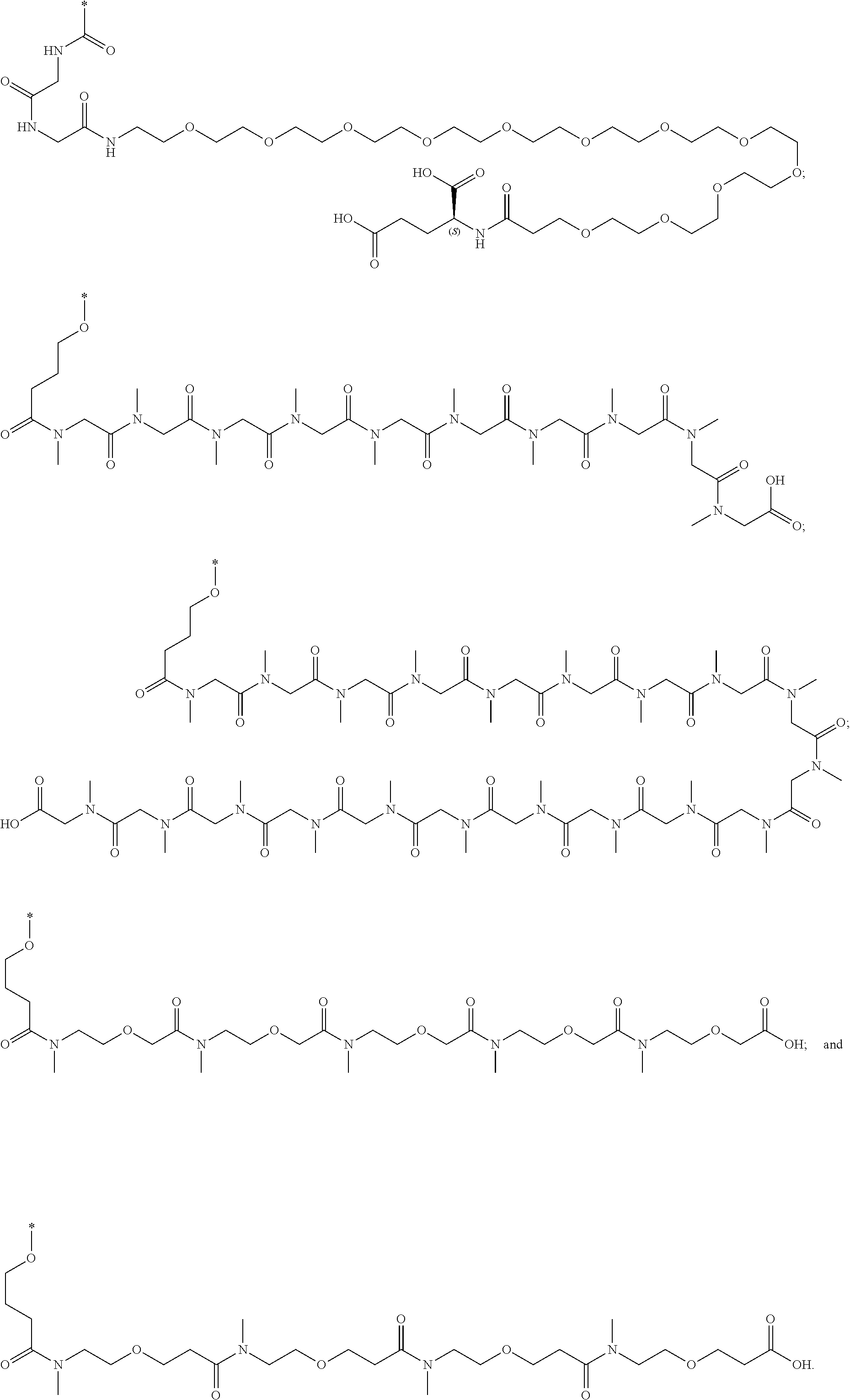

wherein * indicates the point of attachment to Str

An exemplary embodiment of the IGAC of Formula I includes wherein L is a branched linker and PEP is covalently attached to: (i) Str and TNM or IG; and (ii) a solubilizing unit comprises a group selected from polyglycine, polysarcosine, polyethyleneoxy (PEG), C₁-C₄₀ heteroalkyl, and a glycoside, or combinations thereof, wherein the terminus of the solubilizing unit is a group selected from an amino acid, amino, hydroxyl, hydrogen, carboxylic acid, glycerol, or a sugar such as pentaerythritol, maltitol, sorbitol, xylitol, erythritol, isomalt, or combinations thereof.

An exemplary embodiment of the IGAC of Formula I includes wherein L is a branched linker and IM is covalently attached to: (i) IG; and (ii) a solubilizing unit comprises a group selected from polyglycine, polysarcosine, polyethyleneoxy (PEG), C₁-C₄₀ heteroalkyl, and a glycoside, or combinations thereof, wherein the terminus of the solubilizing unit is a group selected from an amino acid, amino, hydroxyl, hydrogen, carboxylic acid, glycerol, or a sugar such as pentaerythritol, maltitol, sorbitol, xylitol, erythritol, isomalt, or combinations thereof.

An exemplary embodiment of the IGAC of Formula I includes wherein Str has the structure:

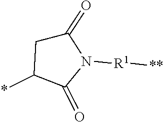

wherein:
* indicates the point of attachment to a cysteine thiol of Ab;
** indicates the point of attachment to PEP or to the isoindolinone-glutarimide moiety;
R¹ is selected from the group consisting of C₁-C₁₂ alkyldiyl, C₁-C₁₂ alkyldiyl-C(═O), C₁-C₁₂alkyldiyl-NH, (CH₂CH₂O)r, (CH₂CH₂O)r-C(═O), (CH₂CH₂O)r-CH₂, C₆-C₂₀ aryldiyl, (C₆-C₂₀ aryldiyl)-(C₁-C₁₂ alkyldiyl), and (C₆-C₂₀ aryldiyl)-(C₁-C₁₂ heteroalkyldiyl);
r is an integer ranging from 1 to 10; and
alkyldiyl, heteroalkyldiyl, and aryldiyl are independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, —S(O)₃H, and a solubilizing unit.

An exemplary embodiment of the IGAC of Formula I includes wherein R¹ is selected from —(CH₂)₅—, and —CH₂CH₂—.

An exemplary embodiment of the IGAC of Formula I includes wherein R¹ is selected from C₆-C₂₀ aryldiyl, (C₆-C₂₀ aryldiyl)-(C₁-C₁₂ alkyldiyl), and (C6-C₂₀ aryldiyl)-(C₁-C₁₂ heteroalkyldiyl).

An exemplary embodiment of the IGAC of Formula I includes wherein Str is selected from the structure:

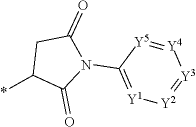

wherein:
one of Y¹, Y², Y³, Y⁴ and Y⁵ is C-L¹-R¹, another one of Y¹, Y², Y³, Y⁴ and Y⁵ is C-L²-R², and the remaining three of Y¹, Y², Y³, Y⁴ and Y⁵ are each independently N, C—R³, or C-L³-R^{3a};
R¹, R², and R^{3a} each independently comprise one or more moieties selected from an amino acid element, a charged element, a heteroalkylene element, a hydrophilic element, a trigger element, an immolative unit, a polar cap, a isoindolinone-glutarimide moiety, and combinations thereof;
   provided that at least one of R¹ and R² comprises the isoindolinone-glutarimide moiety;
each occurrence of R³ is independently selected from the group consisting of hydrogen, deuterium, alkyl, haloalkyl, halo, alkoxy, haloalkoxy, amino, aminyl, amidyl, aldehyde, hydroxyl, cyano, nitro, thiol, carboxy, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide, and a carbohydrate;
R^{4a} and R^{4b} are each independently hydrogen, deuterium, halo, or —S—R^{4c} wherein R^{4c} is substituted or unsubstituted C₆-C₁₀ aryl or substituted or unsubstituted 5-12 membered heteroaryl; and
L¹, L², and L³ are each independently a linker comprising an optionally substituted alkylene, an optionally substituted alkenylene, an optionally substituted alkynylene, an optionally substituted heteroalkylene, an optionally substituted heteroalkenylene, an optionally substituted heteroalkynylene, a heteroatomic linker, an optionally substituted cycloalkylene, an optionally substituted arylene, an optionally substituted heterocyclylene, an optionally substituted heteroarylene, or combinations thereof; and
* indicates the point of attachment to a cysteine thiol of Ab.

An exemplary embodiment of the IGAC of Formula I includes wherein Str is selected from the structure:

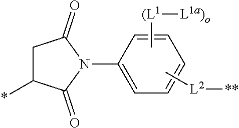

wherein:
L¹ is selected from a bond, C₁-C₁₂ alkyldiyl, and C₁-C₄₀ heteroalkyldiyl;
L¹a is selected from an amino acid, amino, hydroxyl, halide, hydrogen, carboxylic acid, glycerol, or a sugar such as pentaerythritol, maltitol, sorbitol, xylitol, erythritol, isomalt, or combinations thereof;
o is 0, 1, or 2;
L² is selected from a bond, C₁-C₁₂ alkyldiyl, and C₁-C₄₀ heteroalkyldiyl;
* indicates the point of attachment to a cysteine thiol of Ab; and
** indicates the point of attachment to PEP or to the isoindolinone-glutarimide moiety.

An exemplary embodiment of the IGAC of Formula I includes wherein L¹ or L2 comprise a solubilizing unit selected from polyglycine, polysarcosine, polyethyleneoxy (PEG), C₁-C₄₀ heteroalkyl, and a glycoside, or combinations thereof.

An exemplary embodiment of the IGAC of Formula I includes wherein L¹ or L² comprises glucuronic acid having the structure:

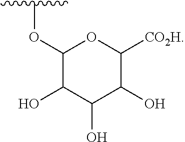 [SMILES: *OC1OC(C(=O)O)C(O)C(O)C1O]

An exemplary embodiment of the IGAC of Formula I includes wherein L¹ or L² is selected from (N(CH₃)CH₂C(═O))_{q}, (N(CH₃)CH₂CH₂C(═O))_{q}, N(CH₃)CH₂CH₂OCH₂CH₂C(═O))_{q}, (CH₂CH₂O)q, (CH₂CH₂O)_{q}C(═O), and (CH₂CH₂O)_{q}—CH₂, where q is an integer from 2 to 20.

An exemplary embodiment of the IGAC of Formula I is selected from formulas:

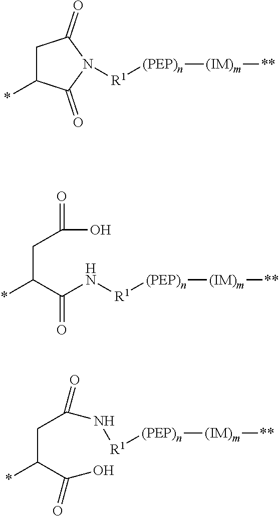

wherein * indicates the point of attachment to a cysteine thiol of Ab and ** indicates the point of attachment to IG.

An exemplary embodiment of the IGAC of Formula I includes wherein n is 1, m is 1, and PEP-IM has the formula:

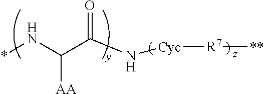

wherein * indicates the point of attachment to Str and ** indicates the point of attachment to IG;
AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and glucuronic acid having the structure:

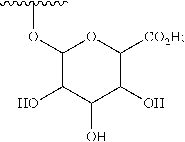 [SMILES: *OC1OC(C(=O)O)C(O)C(O)C1O]

R⁷ is selected from the group consisting of —CH(R⁸)O—, —CH₂—, —CH₂N(R⁸)CH(R⁸)—, —CH(R⁸)OC(═O)—, —CH(R⁸)OC(═O)N(R⁸)CH(R⁸)—, —CH(R⁸)OP(═O)₂OCH(R⁸)—, and —CH(R⁸)OC(═O)N(R⁸)—(C₁-C₆ alkyldiyl)-N(R⁸)C(═O)OCH(R⁸)—;
R⁸ is selected from H, C₁-C₆ alkyl, C(═O)—C₁-C₆ alkyl, and —C(═O)N(R⁹)₂;
R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12; and
z is 0 or 1.

An exemplary embodiment of the IGAC of Formula I includes wherein AA is independently selected from the side chain of a naturally occurring amino acid.

An exemplary embodiment of the IGAC of Formula I includes wherein AA is independently selected from H, —CH₃, —CH(CH₃)₂, —CH₂(C₆H₅), —CH₂C(O)NH₂, —CH₂CH₂CO2H, —CH₂CO2H, —CH₂CH₂CH₂CH₂NH₂, —CH₂CH₂CH₂NHC(NH)NH₂, —CH₂CH(CH₃)₂, —CH₂SO₃H, and —CH₂CH₂CH₂NHC(O)NH₂; or two AA form a 5-membered ring proline amino acid.

An exemplary embodiment of the IGAC of Formula I includes wherein PEP-IM comprises at least one natural or unnatural amino acid side chain AA substituted with C₁-C₄₀ heteroalkyl.

An exemplary embodiment of the IGAC of Formula I includes wherein C₁-C₄₀ heteroalkyl comprises a solubilizing unit selected from polyglycine, polysarcosine, polyethyleneoxy (PEG), and a glycoside, or combinations thereof.

An exemplary embodiment of the IGAC of Formula I includes wherein the solubilizing unit is selected from (N(CH₃)CH₂C(═O))_{q}, (N(CH₃)CH₂CH₂C(═O))_{q}, N(CH₃)CH₂CH₂OCH₂CH₂C(═O))_{q}, (CH₂CH₂O)_{q}, (CH₂CH₂O)_{q}—C(═O), and (CH₂CH₂O)_{q}—CH₂, where q is an integer from 2 to 20.

An exemplary embodiment of the IGAC of Formula I includes wherein y is 2, PEP is a dipeptide, and PEP-IM has the formula:

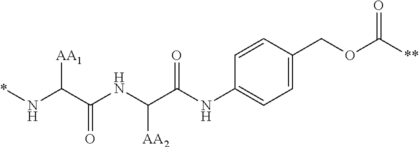

wherein AA₁ and AA₂ are independently selected from a side chain of a naturally-occurring amino acid.

An exemplary embodiment of the IGAC of Formula I includes wherein the dipeptide is selected from ala-ala, val-cit, and phe-ala.

An exemplary embodiment of the IGAC of Formula I includes wherein AA₁ is —CH(CH₃)₂, and AA₂ is —CH₂CH₂CH₂NHC(O)NH₂.

An exemplary embodiment of the IGAC of Formula I includes wherein AA₁ and AA₂ are each —CH₃.

An exemplary embodiment of the IGAC of Formula I includes wherein y is 3, PEP is a tripeptide, and PEP-IM has the formula:

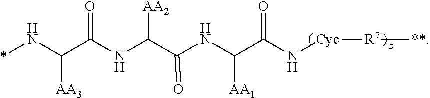

An exemplary embodiment of the IGAC of Formula I includes wherein y is 4, PEP is a tetrapeptide PEP-IM, and has the formula:

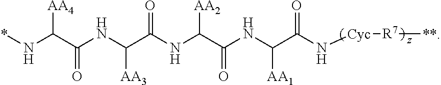

An exemplary embodiment of the IGAC of Formula I includes where z is 1 and IM has the formula:

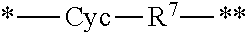

wherein:
* indicates the point of attachment to PEP; and
** indicates the point of attachment to IG.

An exemplary embodiment of the IGAC of Formula I includes where R⁷ has the formula:

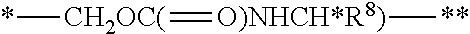

wherein:
R⁸ is selected from H and C₁-C₆ alkyl;
* indicates the point of attachment to PEP; and
** indicates the point of attachment to IG.

An exemplary embodiment of the IGAC of Formula I includes where R⁸ is H.

An exemplary embodiment of the IGAC of Formula I includes where R⁸ is —CH₃.

An exemplary embodiment of the IGAC of Formula I includes wherein IM is selected from the formulae:

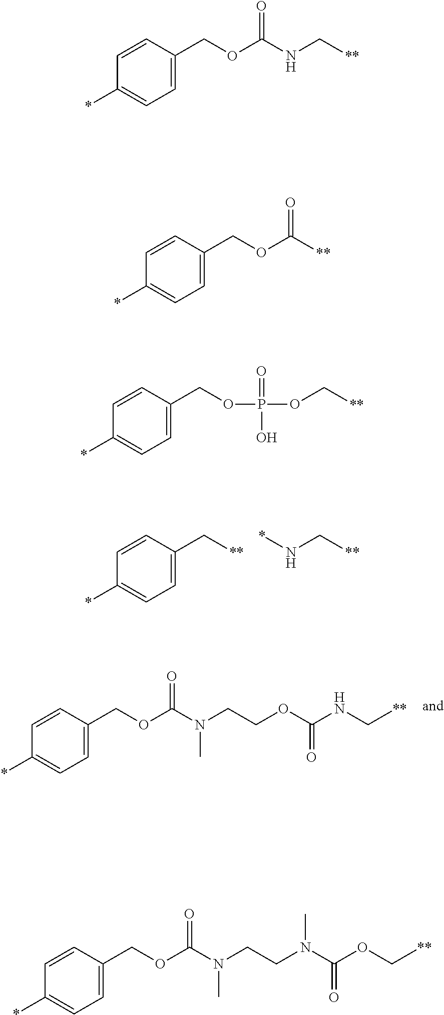

wherein:
* indicates the point of attachment to PEP; and
** indicates the point of attachment to IG.

An exemplary embodiment of the IGAC of Formula I includes wherein IM is selected from the formulae:

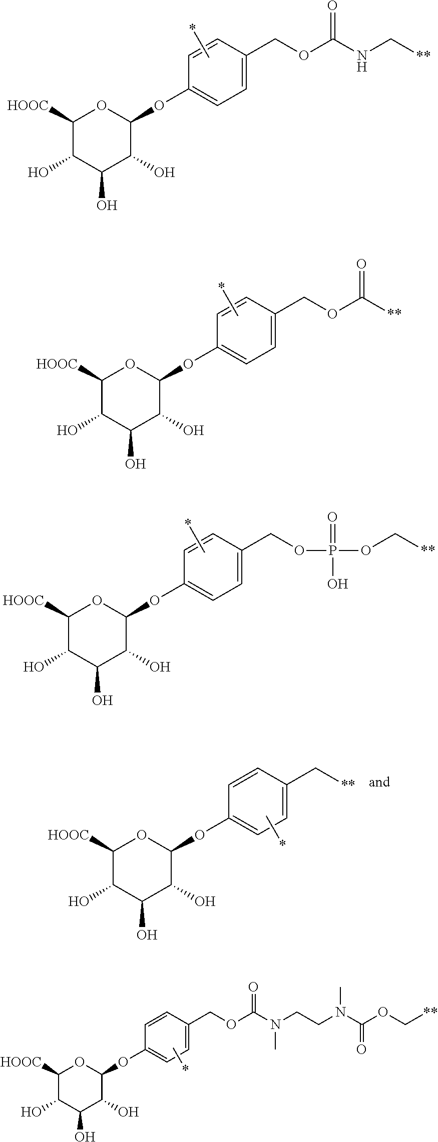

wherein:
* indicates the point of attachment to PEP; and
** indicates the point of attachment to IG.

The invention includes all reasonable combinations, and permutations of the features, of the Formula I embodiments.

In certain embodiments, the isoindolinone-glutarimide antibody conjugates (IGAC) of the invention include those with anti-cancer activity. The IGAC selectively deliver an effective dose of an isoindolinone-glutarimide drug to tumor tissue or hematopoietic cell, whereby greater selectivity (i.e., a lower efficacious dose) may be achieved while increasing the therapeutic index (“therapeutic window”) relative to unconjugated isoindolinone-glutarimide drug.

Drug loading in Table 3 is represented as DAR, the number of isoindolinone-glutarimide (IG) moieties per antibody in an IGAC of Formula I. Drug (IG) loading may range from 1 to about 8 drug moieties (D) per antibody. IGAC of Formula I include mixtures or collections of antibodies conjugated with a range of IG drug moieties, from 1 to about 8. In some embodiments, the number of IG drug moieties that can be conjugated to an antibody is limited by the number of reactive or available amino acid side chain residues such as lysine and cysteine. In some embodiments, free cysteine residues are introduced into the antibody amino acid sequence by the methods described herein. In such aspects, p may be 1, 2, 3, 4, 5, 6, 7, or 8, and ranges thereof, such as from 1 to 8 or from 2 to 5. Exemplary IGAC of Formula I include, but are not limited to, antibodies that have 1, 2, 3, or 4 engineered cysteine amino acids (Lyon, R. et al. (2012) Methods in Enzym. 502:123-138). In some embodiments, one or more free cysteine residues are already present in an antibody forming intra-chain and inter-chain disulfide bonds (native disulfide groups), without the use of engineering, in which case the existing free, reduced cysteine residues may be used to conjugate the antibody to a drug. In some embodiments, an antibody is exposed to reducing conditions prior to conjugation of the antibody in order to generate one or more free cysteine residues.

For some antibody conjugates, p may be limited by the number of attachment sites on the antibody. For example, where the attachment is a cysteine thiol, as in certain exemplary embodiments described herein, an antibody may have only one or a limited number of cysteine thiol groups, or may have only one or a limited number of sufficiently reactive thiol groups, to which the drug may be attached. In other embodiments, one or more lysine amino groups in the antibody may be available and reactive for conjugation with a IG-linker compound of Formula IL In certain embodiments, higher drug loading, e.g. p >5, may cause aggregation, insolubility, toxicity, or loss of cellular permeability of certain antibody-drug conjugates. In certain embodiments, the average drug loading for an antibody conjugate ranges from 1 to about 8; from about 2 to about 6; or from about 3 to about 5. In certain embodiments, an antibody is subjected to denaturing conditions to reveal reactive nucleophilic groups such as lysine or cysteine.

The loading (drug/antibody ratio) of an antibody conjugate may be controlled in different ways, and for example, by: (i) limiting the molar excess of the IG-linker intermediate compound relative to antibody, (ii) limiting the conjugation reaction time or temperature, and (iii) partial or limiting reductive denaturing conditions for optimized antibody reactivity.

It is to be understood that where more than one nucleophilic group of the antibody reacts with a drug, then the resulting product is a mixture of antibody conjugate compounds with a distribution of one or more IG drug moieties attached to an antibody. The average number of drugs per antibody may be calculated from the mixture by a dual ELISA antibody assay, which is specific for antibody and specific for the drug. Individual IGAC molecules may be identified in the mixture by mass spectroscopy and separated by HPLC, for example hydrophobic interaction chromatography, HIC (McDonagh et al. (2006) Prot. Engr. Design & Selection 19(7):299-307; Hamblett et al. (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, March 27-31, 2004, Proceedings of the AACR, Volume 45, March 2004). In certain embodiments, a homogeneous antibody conjugate with a single loading value may be isolated from the conjugation mixture by electrophoresis or chromatography.

Exemplary embodiments of the isoindolinone-glutarimide antibody conjugates (IGAC) of Formula I are compiled in Table 3. Assessment of IGAC biological activity and other properties may be conducted according to the methods of Example 102. In some instances, the Cell proliferation dose response IC50 value is an average of repetitive assays. Certain exemplary IGAC were tested for their effects in inhibiting cellular proliferation, including CAL51; WSU-DLCL2, NCI-N87 and SKBR3 cell lines. CAL51 is a human breast adenocarcinoma cell line with triple-negative status for expression of estrogen, progesterone and HER2 receptors. WSU-DLCL2 is a human B-Cell non-Hodgkin lymphoma cell line that expresses high levels of CD22. NCI-N87 is a human epithelial cell line established from a gastric carcinoma; SKBR3 is a human epithelial cell line established from a breast adenocarcinoma; both NCI-N87 and SKBR3 cell lines express high levels of HER2 receptor.

TABLE 3
Isoindolinone-glutarimide antibody conjugates (IGAC)

| | | | | | Cell | |
|---|---|---|---|---|---|---|
| | | | | Cell | proliferation | Cell |
| | | | | proliferation | dose response | proliferation |
| | IG-L No. | | | dose response | IC50 (nM) | dose response |
| IGAC | linker- | Antibody | | IC50 (nM) | WSU- | IC50 (nM) |
| No. | payload | Antigen | DAR | SKBR3 | DCLC2 | CAL51 |
| IGAC-1 | IG-L-1 | trastuzumab | 3.9, 3.6 | 0.043 | >66.7 | >100 |
| | | HER2 | | | | |
| IGAC-2 | IG-L-1 | isotype control | 3.7 | 202 | | >1000 |
| IGAC-3 | IG-L-6 | trastuzumab | 4.4 | 0.052 | | 18.4 |
| | | HER2 | | | | |
| IGAC-4 | IG-L-6 | isotype control | 4.1 | 127 | | 254 |
| IGAC-5 | IG-L-2 | trastuzumab | 7.9 | 0.051 | >66.7 | >100 |
| | | HER2 | | | | |
| IGAC-6 | IG-L-2 | isotype control | 8.0 | 79.8 | | 82.6 |
| IGAC-7 | IG-L-6 | trastuzumab | 7.8 | 0.109 | >66.7 | >100 |
| | | HER2 | | | | |
| IGAC-8 | IG-L-6 | isotype control | 8.0 | 21.5 | 178 | 114 |
| IGAC-9 | IG-L-1 | trastuzumab | 7.8 | 0.029 | >66.7 | >100 |
| | | HER2 | | | | |
| IGAC-10 | IG-L-1 | isotype control | 8.0 | 75.65 | 683 | 760 |
| IGAC-11 | IG-L-3 | trastuzumab | 7.9 | >66.7 | >66.7 | |
| | | HER2 | | | | |
| IGAC-12 | IG-L-3 | isotype control | 8.0 | >1000 | | >1000 |
| IGAC-13 | IG-L-7 | trastuzumab | 7.9 | 0.046 | >66.7 | >66.7 |
| | | HER2 | | | | |
| IGAC-14 | IG-L-7 | isotype control | 8.0 | 203.7 | | >1000 |
| IGAC-15 | IG-L-4 | trastuzumab | 8.0 | 0.144 | >66.7 | >100 |
| | | HER2 | | | | |
| IGAC-16 | IG-L-4 | isotype control | 8.0 | >1000 | | >1000 |
| IGAC-17 | IG-L-5 | trastuzumab | 7.7 | >100 | | >100 |
| | | HER2 | | | | |
| IGAC-18 | IG-L-5 | isotype control | 8.0 | >1000 | | >1000 |
| IGAC-19 | IG-L-9 | trastuzumab | 8.0 | 0.291 | | >100 |
| | | HER2 | | | | |
| IGAC-20 | IG-L-9 | isotype control | 8.0 | >1000 | | >1000 |
| IGAC-21 | IG-L-10 | trastuzumab | 8.0 | 8.39 | | >100 |
| | | HER2 | | | | |
| IGAC-22 | IG-L-10 | isotype control | 8.0 | 17.7 | | >1000 |
| IGAC-23 | IG-L-11 | trastuzumab | 8.0 | >100 | | >100 |
| | | HER2 | | | | |
| IGAC-24 | IG-L-11 | isotype control | 8.0 | >1000 | | >1000 |
| IGAC-25 | IG-L-6 | pinatuzumab | 7.9 | | 0.0786 | |
| | | CD22 | | | | |
| IGAC-26 | IG-L-6 | gemtuzumab | 8.0 | | | |
| | | CD33 | | | | |
| IGAC-27 | IG-L-1 | pinatuzumab | 3.8 | >66.7 (BJAB) | 0.307 | |
| | | CD22 | | | | |
| IGAC-28 | IG-L-1 | gemtuzumab | 3.9 | | | |
| | | CD33 | | | | |
| IGAC-29 | IG-L-12 | trastuzumab | 8.0 | 0.563 | | >66.7 |
| | | HER2 | | | | |
| IGAC-30 | IG-L-12 | isotype control | 8.0 | 14.6 | | 124 |
| IGAC-31 | IG-L-14 | trastuzumab | 7.7 | 0.17 | | >66.7 |
| | | HER2 | | | | |
| IGAC-32 | IG-L-1 | gemtuzumab | 7.9 | | | |
| | | CD33 | | | | |
| IGAC-33 | IG-L-16 | trastuzumab | 8.0 | 0.0752 | | >1000 |
| | | HER2 | | | | |
| IGAC-34 | IG-L-14 | pinatuzumab | 7.9 | >66.7 (BJAB) | 0.106 | |
| | | CD22 | | | | |
| IGAC-35 | IG-L-17 | trastuzumab | 8.0 | >66.7 | | >1000 |
| | | HER2 | | | | |
| IGAC-36 | IG-L-13 | trastuzumab | 8.0 | 1.47 | | 27.4 |
| | | HER2 | | | | |
| IGAC-37 | IG-L-15 | trastuzumab | | 27 | | |
| | | HER2 | | | | |
| IGAC-38 | IG-L-16 | pinatuzumab | 8.0 | >66.7 | 0.141 | |
| | | CD22 | | | | |
| IGAC-39 | IG-L-6 | trastuzumab | 1.9 | 0.432 | | |
| | | HER2 | | | | |
| IGAC-40 | IG-L-18 | trastuzumab | 7.8 | 0.155 | | |
| | | HER2 | | | | |
| IGAC-41 | IG-L-19 | trastuzumab | 7.9 | 0.0612 | | |
| | | HER2 | | | | |
| IGAC-42 | IG-L-20 | trastuzumab | 7.9 | 0.0410 | >66.7 | >66.7 |
| | | HER2 | | | | |
| IGAC-43 | IG-L-21 | trastuzumab | 7.9 | 0.0395 | | >66.7 |
| | | HER2 | | | | |
| IGAC-44 | IG-L-22 | trastuzumab | 7.9 | 0.0241 | | >66.7 |
| | | HER2 | | | | |
| IGAC-45 | IG-L-24 | pinatuzumab | 3.9 | 4.88 (OCI- | | |
| | | CD22 | | LY19) | | |
| IGAC-46 | IG-L-26 | pinatuzumab | 7.9 | 43.1 | 43.6 | |
| | | CD22 | | | | |
| IGAC-47 | IG-L-26 | trastuzumab | 7.9 | 14.2 | | >66.7 |
| | | HER2 | | | | |
| IGAC-48 | IG-L-23 | trastuzumab | 7.9 | 0.632 | | 4.86 |
| | | HER2 | | | | |
| IGAC-49 | IG-L-25 | trastuzumab | 7.9 | 0.022 | >66.7 | >66.7 |
| | | HER2 | | | | |
| IGAC-50 | IG-L-36 | trastuzumab | 7.8 | | 55.9 | |
| | | HER2 | | | | |
| IGAC-51 | IG-L-36 | pinatuzumab | 7.9 | | 14.0 | |
| | | CD22 | | | | |
| IGAC-52 | IG-L-37 | trastuzumab | | | | |
| | | HER2 | | | | |
| IGAC-53 | IG-L-37 | pinatuzumab | 7.7 | >66.7 | 0.525 | |
| | | CD22 | | | | |
| IGAC-54 | IG-L-38 | Trastuzumab | 8 | 0.133 | >66.7 | 0.305 (NCI- |
| | | HER2 | | | | N87) |
| IGAC-55 | IG-L-39 | Trastuzumab | 8.0 | 0.161 | >66.7 | 0.314 (NCI- |
| | | HER2 | | | | N87) |
| IGAC-56 | IG-L-47 | Trastuzumab | 7.9 | 0.060 | >66.7 | 0.132 (NCI- |
| | | HER2 | | | | N87) |
| IGAC-57 | IG-L-62 | Trastuzumab | 8 | 0.039 | >66.7 | 0.111 (NCI- |
| | | HER2 | | | | N87) |
| IGAC-58 | IG-L-63 | Trastuzumab | 7.9 | 0.022 | >66.7 | 0.085 (NCI- |
| | | HER2 | | | | N87) |
| IGAC-59 | IG-L-63 | Pinatuzumab | 7.9 | >66.7 | 0.185 | |
| | | CD22 | | | | |
| IGAC-60 | IG-L-54 | Pinatuzumab | 7.9 | >66.7 | 1.37 | |
| | | CD22 | | | | |
| IGAC-61 | IG-L-58 | Trastuzumab | 7.9 | 0.061 | >66.7 | 0.102 (NCI- |
| | | HER2 | | | | N87) |
| IGAC-62 | IG-L-58 | Pinatuzumab | 7.9 | >66.7 | 0.379 | |
| | | CD22 | | | | |
| IGAC-63 | IG-L-46 | trastuzumab | 7.9 | 0.0858 | 55.8 | |
| | | HER2 | | | | |
| IGAC-64 | IG-L-46 | pinatuzumab | 7.9 | >66.7 | 11.5 | |
| | | CD22 | | | | |
| IGAC-65 | IG-L-65 | trastuzumab | 8.0 | 0.0226 | >66.7 | |
| | | HER2 | | | | |
| IGAC-66 | IG-L-65 | pinatuzumab | 8.0 | >66.7 | 5.44 | |
| | | CD22 | | | | |
| IGAC-67 | IG-L-47 | pinatuzumab | 7.9 | >66.7 | 1.34 | |
| | | CD22 | | | | |
| IGAC-68 | IG-L-71 | trastuzumab | | | | |
| | | HER2 | | | | |
| IGAC-69 | IG-L-68 | trastuzumab HC | 2.0 | | | |
| | | S375C | | | | |
| | | HER2 | | | | |
| IGAC-70 | IG-L-68 | pinatuzumab HC | 2.0 | | | |
| | | S375C | | | | |
| | | CD22 | | | | |
| IGAC-71 | IG-L-70 | trastuzumab HC | 2.0 | | | |
| | | S375C | | | | |
| | | HER2 | | | | |
| IGAC-72 | IG-L-70 | pinatuzumab HC | 2.0 | | | |
| | | S375C | | | | |
| | | CD22 | | | | |
| IGAC-73 | IG-L-48 | trastuzumab | 7.8 | >66.7 | >66.7 | |
| | | HER2 | | | | |
| IGAC-74 | IG-L-48 | pinatuzumab | 8.0 | >66.7 | >66.7 | |
| | | CD22 | | | | |
| IGAC-75 | IG-L-45 | trastuzumab | 7.9 | 0.0166 | >66.7 | |
| | | HER2 | | | | |
| IGAC-76 | IG-L-45 | pinatuzumab | | | | |
| | | CD22 | | | | |
| IGAC-77 | IG-L-40 | trastuzumab | 8.0 | 0.241 | >66.7 | |
| | | HER2 | | | | |
| IGAC-78 | IG-L-40 | pinatuzumab | 7.9 | >66.7 | 0.943 | |
| | | CD22 | | | | |
| IGAC-79 | IG-L-42 | trastuzumab | 8.0 | 0.0646 | >66.7 | |
| | | HER2 | | | | |
| IGAC-80 | IG-L-42 | pinatuzumab | 8.0 | >66.7 | 0.466 | |
| | | CD22 | | | | |
| IGAC-81 | IG-L-41 | trastuzumab | 8.0 | 0.194 | >66.7 | 0.319 (NCI- |
| | | HER2 | | | | N87) |
| IGAC-82 | IG-L-41 | pinatuzumab | 7.8 | >66.7 | 5.61 | |
| | | CD22 | | | | |
| IGAC-83 | IG-L-39 | pinatuzumab | 7.9 | >66.7 | 8.85 | |
| | | CD22 | | | | |
| IGAC-84 | IG-L-43 | trastuzumab | 8.0 | 0.229 | >66.7 | 0.547 (NCI- |
| | | HER2 | | | | N87) |
| IGAC-85 | IG-L-44 | trastuzumab | 8.0 | >66.7 | >66.7 | |
| | | HER2 | | | | |
| IGAC-86 | IG-L-38 | pinatuzumab | 8.0 | | | |
| | | CD22 | | | | |
| IGAC-87 | IG-L-25 | h7G3 | 8.0 | | | |
| | | CD123 | | | | |
| IGAC-88 | IG-L-25 | loncastuximab | 8.0 | | | |
| | | CD19 | | | | |
| IGAC-89 | IG-L-25 | gemtuzumab | 8.0 | | | |
| | | CD33 | | | | |
| IGAC-90 | IG-L-25 | MCLL0517A | 8.0 | | | |
| | | CLL-1 | | | | |
| | | (CLEC12A) | | | | |
| IGAC-91 | IG-L-25 | mirvetuximab | | | | |
| | | FOLR1 | | | | |
| IGAC-92 | IG-L-25 | rospatamab | 8.0 | | | |
| | | PSMA | | | | |
| IGAC-93 | IG-L- | trastuzumab | 3.5 | 8.72 | | |
| | | HER2 | | | | |
| IGAC-94 | IG-L-25 | pivekimab | 8.0 | | | |
| | | CD123 | | | | |
| IGAC-95 | IG-L-25 | CHv62.21 | 8.0 | | | |
| | | FLT3 | | | | |
| IGAC-96 | IG-L-73 | trastuzumab | 7.9 | 0.0558 | 59.2 | |
| | | HER2 | | | | |
| IGAC-97 | IG-L-74 | trastuzumab | 7.9 | 0.0313 | >66.7 | |
| | | HER2 | | | | |
| IGAC-98 | IG-L-75 | trastuzumab | | | | |
| | | HER2 | | | | |
| IGAC-99 | IG-L-76 | trastuzumab | | | | |
| | | HER2 | | | | |
| IGAC-100 | IG-L-77 | trastuzumab | | | | |
| | | HER2 | | | | |
| IGAC-101 | IG-L-78 | trastuzumab | | | | |
| | | HER2 | | | | |
| IGAC-102 | IG-L-79 | trastuzumab | | | | |
| | | HER2 | | | | |
| IGAC-103 | IG-L-80 | trastuzumab | | | | |
| | | HER2 | | | | |
| IGAC-104 | IG-L-81 | trastuzumab | | | | |
| | | HER2 | | | | |
| IGAC-105 | IG-L-82 | trastuzumab | | | | |
| | | HER2 | | | | |

### Pharmaceutical Compositions of Isoindolinone-Glutarimide Antibody Conjugates

The invention provides a composition, e.g., a pharmaceutically or pharmacologically acceptable composition or formulation, comprising an isoindolinone-glutarimide antibody conjugate (IGAC) composition of the invention as described herein and a pharmaceutically acceptable diluent, vehicle, carrier or excipient.

The isoindolinone-glutarimide antibody conjugate (IGAC) composition can be the same or different in the pharmaceutical composition, i.e., the composition can comprise IGAC that have the same number of isoindolinone-glutarimide (IC) moieties linked to the same positions on the antibody and/or IGAC that have the same number of (IG) moieties linked to different positions on the antibody, that have different numbers of (IG) moieties linked to the same positions on the antibody, or that have different numbers of (IG) moieties linked to different positions on the antibody. In an exemplary embodiment, a pharmaceutical composition comprises a mixture of the IGAC, wherein the average drug (IG) loading per antibody in the mixture of antibody conjugate compounds is about 2 to about 8.

An IGAC of the invention can have an average IG to antibody ratio (DAR) of about 0.4 to about 10. A skilled artisan will recognize that the number of IG moieties conjugated to the antibody may vary amongst IGAC in a composition comprising multiple IGAC of the invention and thus the DAR can be measured as an average which may be referred to as the drug to antibody ratio (DAR) which can be assessed by any suitable means, many of which are known in the art. The average number of IG moieties per antibody (DAR) in preparations of IGAC from conjugation reactions may be characterized by conventional means such as mass spectrometry, ELISA assay, and HPLC. The quantitative distribution of IGAC in a composition in terms of p may also be determined. In certain instances, separation, purification, and characterization of homogeneous IGAC where p is a certain value from antibody drug conjugates (ADC) with other drug loadings may be achieved by purification means such as reverse phase HPLC or electrophoresis.

An isoindolinone-glutarimide antibody conjugate (IGAC) composition can be formulated for parenteral administration, such as intradermal, subcutaneous, intramuscular (IM), or intravenous (IV) injections, infusion, or administration into a body cavity or lumen of an organ. Alternatively, the IGAC as a pharmaceutical composition can be injected into otherwise placed into a specific site of the body, such as a tumor. Compositions for injection will commonly comprise a solution of the IGAC dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These pharmaceutical compositions desirably are sterile and generally free of undesirable matter. These pharmaceutical compositions can be made sterilized by conventional, well known sterilization techniques. The pharmaceutical compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The pharmaceutical composition may contain any suitable concentration of the IGAC. The concentration of the IGAC in the pharmaceutical composition can vary widely, and will be selected primarily based on fluid volumes, viscosities, body weight, and the like, in accordance with the particular mode of administration selected and the patient's needs. In certain embodiments, the concentration of IGAC in a solution formulation for injection will range from about 0.1% (w/w) to about 10% (w/w).

Methods of Treating Cancer with Isoindolinone-Glutarimide Antibody Conjugates

By inducing target-specific degradation of tumor-associated proteins and conferring specificity to minimize off-target toxicity effects, the isoindolinone-glutarimide antibody conjugate (IGAC) compositions of the invention may be useful in the treatment of diseases and disorders such as cancer. The IGAC direct a tumor-associated antigen-binding antibody to a cell that expresses the antigen and deliver a cereblon-degrading moiety to the target cell. A target protein such as GSPT1 is ubiquitinated and subsequently degraded.

The invention provides a method for treating cancer with a pharmaceutical composition of the IGAC. The method includes administering a therapeutically effective amount of an antibody conjugate composition as described herein to a subject in need thereof, such as a patient that has cancer and is in need of treatment for the cancer. The method includes administering a therapeutically effective amount of an IGAC selected from Table 3.

In certain embodiments, the IGAC include those with anticancer activity (FIGS. 1-4). The IGAC selectively delivers an effective dose of an active form of the isoindolinone-glutarimide protein target degrader moiety to tumor tissue, whereby greater selectivity (i.e., a lower efficacious dose) may be achieved while increasing the therapeutic index (“therapeutic window”) relative to an unconjugated protein target degrader compound.

It is contemplated that the IGAC may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

Examples of cancer to be treated herein include, but are not limited to, carcinoma, lymphoma, blastoma, sarcoma, leukemia or lymphoid malignancies including acute myeloid leukemia, squamous cell cancer, epithelial squamous cell cancer, lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer. In some embodiments, the IGAC may be useful in therapy to treat solid tumors such as lung cancer; non-small cell lung cancer, squamous cell lung cancer, small cell lung cancer, breast cancer, and neuroendocrine cancers such as neuroendocrine prostate cancer, castration-resistant neuroendocrine prostate cancer (NEPC) and lung neuroendocrine tumors. In some embodiments, the IGAC may be useful in therapy to treat blood-borne hematological cancers such as leukemias; acute myelogenous leukemia (AML) and myelomas; multiple myeloma (MM).

In another aspect, an IGAC for use as a medicament is provided. In certain embodiments, the invention provides an IGAC for use in a method of treating an individual comprising administering to the individual an effective amount of the antibody conjugate composition in a pharmaceutical composition. In one such embodiment, the method further comprises administering to the individual an effective amount of at least one additional therapeutic agent, e.g., as described herein.

In a further aspect, the invention provides for the use of an IGAC in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. The IGAC dose can range from about 5 mg/kg (body weight) to about 50 mg/kg, from about 10 μg/kg to about 5 mg/kg, or from about 100 g/kg to about 1 mg/kg. The IGAC dose can be about 100, 200, 300, 400, or 500 g/kg. The IGAC dose can be about 1, 2, 3, 4, 5, 6, 7, 8, 9, or 10 mg/kg. The IGAC dose can also be outside of these ranges, depending on the particular conjugate as well as the type and severity of the cancer or disorder being treated. Frequency of administration can range from a single dose to multiple doses per week, or more frequently. In some embodiments, the IGAC is administered from about once per month to about five times per week. In some embodiments, the IGAC is administered once per week.

The IGAC can be used either alone or in combination with other therapeutic agents in a therapy regimen. IGAC may be administered concurrently in a regimen with one or more other drugs during the same treatment cycle, on the same day of treatment as the one or more other drugs, and, optionally, at the same time as the one or more other drugs. For instance, for cancer therapies given every 3 weeks, the concurrently administered drugs are each administered on day-1 of a 3-week cycle. For instance, an IGAC may be co-administered with at least one additional therapeutic agent, such as a chemotherapeutic agent. Such combination therapies encompass combined administration (where two or more therapeutic agents are included in the same or separate formulations), and separate administration, in which case, administration of the IGAC can occur prior to, simultaneously, and/or following, administration of the additional therapeutic agent. IGAC can also be used in combination with radiation therapy.

### EXAMPLES

### General Synthetic Schemes and Examples

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art of prepared as described herein.

### Example 1 Synthesis of 1-(3-chloro-4-methylphenyl)-3-((2-(2,6-dioxopiperidin-3-yl)-1-oxoisoindolin-5-yl)methyl)urea (Compound IG-1)

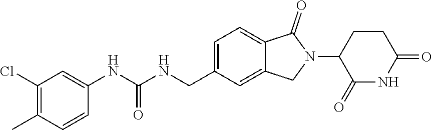 [SMILES: Cc1ccc(NC(=O)NCc2ccc3c(c2)CN(C2CCC(=O)NC2=O)C3=O)cc1Cl]

### Step A. Preparation of Int 1a

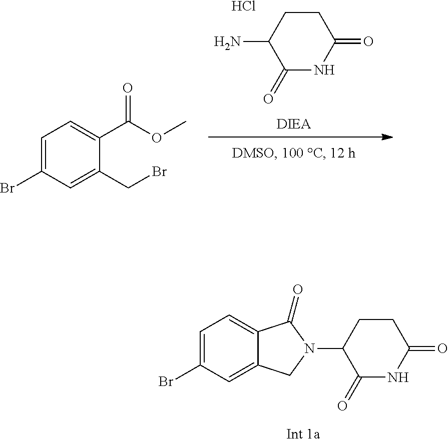

A mixture of methyl 4-bromo-2-(bromomethyl)benzoate (20.0 g, 64.9 mmol, 1.00 eq), 3-aminopiperidine-2,6-dione (12.8 g, 78.0 mmol, 1.20 eq) and N,N-diisopropylethylamine (33.6 g, 259 mmol, 45.2 mL, 4.00 eq) in dimethylsulfoxide (200 mL) was stirred at 100° C. for 12 h. The reaction mixture was poured into water (300 mL) and stirred for 10 min, filtered to give a filter cake, the filter cake was concentrated to give a residue. The residue was triturated with ethyl acetate (100 mL) at 20° C. for 10 min to afford 3-(5-bromo-1-oxoisoindolin-2-yl)piperidine-2,6-dione, Int 1a, (17.0 g, 52.6 mmol, 81% yield) as a purple solid. MS (ESI) m/z 325.0 [M+H+2]⁺

### Step B. Preparation of Int 1b

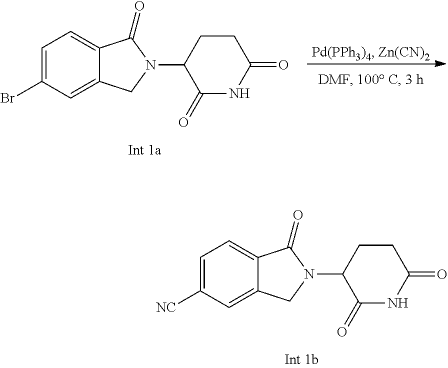

A mixture of 3-(5-bromo-1-oxoisoindolin-2-yl)piperidine-2,6-dione, Int 1a, (500 mg, 1.55 mmol, 1.00 eq), zinc cyanide (110 mg, 936 umol, 59.0 uL, 0.600 eq) and tetrakis[triphenylphosphine]palladium(O) (178 mg, 154 umol, 0.100 eq) in dimethylformamide (10.0 mL) was stirred at 100° C. for 3 h under nitrogen atmosphere. The reaction mixture was cooled to 25° C. and then poured into water (100 mL) and extracted with ethyl acetate (3×50.0 mL). The combined organic phase was washed with brine (100 mL), dried over anhydrous sodium sulfate, filtered and concentrated in vacuum to give a residue. The residue was triturated with ethyl acetate (20.0 mL) at 25° C. for 30 min to afford 2-(2,6-dioxopiperidin-3-yl)-1-oxoisoindoline-5-carbonitrile, Int 1b, (400 mg, crude) as a gray solid. ¹H NMR (400 MHz, DMSO-d₆) δ=11.03 (s, 1H), 8.17 (s, 1H), 8.03-7.97 (m, 1H), 7.95-7.89 (m, 1H), 5.16 (dd, J=5.1, 13.3 Hz, 1H), 4.61-4.52 (m, 1H), 4.49-4.38 (m, 1H), 2.98-2.86 (m, 1H), 2.68-2.59 (m, 1H), 2.46-2.37 (m, 1H), 2.09-1.98 (m, 1H). MS (ESI) m/z 270.2 [M+H]+

### Step C. Preparation of Int AA

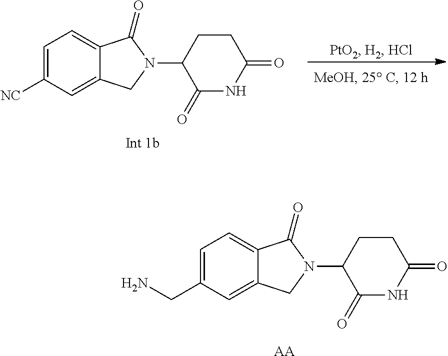

To a solution of 2-(2,6-dioxopiperidin-3-yl)-1-oxoisoindoline-5-carbonitrile, Int 1b, (400 mg, 1.49 mmol, 1.00 eq) in hydrochloric acid (12 M, 1.00 mL, 8.08 eq) and methanol (10.0 mL) was added platinum dioxide (170 mg, 748 umol, 0.500 eq) under nitrogen atmosphere, the mixture was stirred at 25° C. for 12 h under hydrogen atmosphere. The reaction was filtered to give a filtrate, the filtrate was concentrated to give a residue. The residue was triturated with ethyl acetate (30.0 mL) at 25° C. for 10 min to afford 3-(5-(aminomethyl)-1-oxoisoindolin-2-yl)piperidine-2,6-dione, Int AA, (250 mg, 914 umol, 62% yield) as a yellow solid. ¹H NMR (400 MHz, DMSO-d₆) δ=11.00 (br s, 1H), 8.66-8.45 (m, 2H), 7.78 (br d, J=6.4 Hz, 1H), 7.74 (br s, 1H), 7.64 (br d, J=7.7 Hz, 1H), 5.21-5.06 (m, 1H), 4.54-4.46 (m, 1H), 4.39-4.31 (m, 1H), 4.16 (br d, J=1.7 Hz, 2H), 2.90 (br s, 1H), 2.68-2.64 (m, 1H), 2.42 (br d, J=12.1 Hz, 1H), 2.10-1.96 (m, 1H).

### Step D. Preparation of Compound IG-1

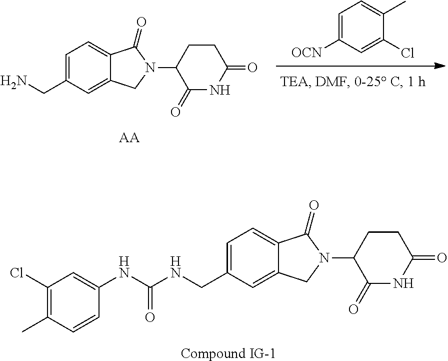

To a solution of 3-(5-(aminomethyl)-1-oxoisoindolin-2-yl)piperidine-2,6-dione, Compound AA, (250 mg, 914 umol, 1.00 eq) and triethylamine (277 mg, 2.74 mmol, 382 uL, 3.00 eq) in dimethylformamide (2.00 mL) was added 2-chloro-4-isocyanato-1-methylbenzene (168 mg, 1.01 mmol, 1.10 eq) at 0° C., the mixture was stirred at 25° C. for 1 h. The reaction mixture was poured into water (50.0 mL) and stirred for 10 min, then filtered to give a filter cake, the filter cake was concentrated to give a residue. The residue was purified by Prep-HPLC (column: Waters Xbridge 150*25 mm*5 um; mobile phase: [water(FA)-ACN]; B %: 11%-41%, 10 min) to afford 1-(3-chloro-4-methylphenyl)-3-((2-(2,6-dioxopiperidin-3-yl)-1-oxoisoindolin-5-yl)methyl)urea (119.12 mg, 267 umol, 29% yield, 99% purity) as a yellow solid. ¹H NMR (400 MHz, DMSO-d₆) δ=10.98 (br s, 1H), 8.77 (s, 1H), 7.73-7.64 (m, 2H), 7.52 (s, 1H), 7.45 (d, J=8.1 Hz, 1H), 7.20-7.17 (m, 1H), 7.16-7.11 (m, 1H), 6.82 (t, J=5.9 Hz, 1H), 5.11 (dd, J=5.1, 13.3 Hz, 1H), 4.49-4.39 (m, 3H), 4.36-4.26 (m, 1H), 2.98-2.87 (m, 1H), 2.64-2.61 (m, 1H), 2.43-2.34 (m, 1H), 2.23 (s, 3H), 2.05-1.96 (m, 1H). MS (ESI) m/z 441.1 [M+H]+

### Example 2 Synthesis of 1-(3-chloro-4-(2-(2-(methylamino)ethoxy)ethyl)phenyl)-3-((2-(2,6-dioxopiperidin-3-yl)-1-oxoisoindolin-5-yl)methyl)urea (Compound IG-3)

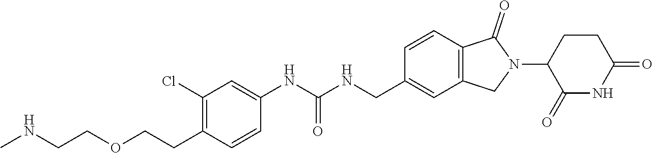 [SMILES: CNCCOCCc1ccc(NC(=O)NCc2ccc3c(c2)CN(C2CCC(=O)NC2=O)C3=O)cc1Cl]

### Step A. Preparation of Int 1c

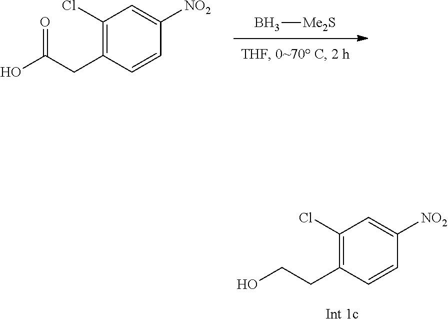

To a solution of 2-(2-chloro-4-nitrophenyl)acetic acid (5.00 g, 23.2 mmol, 1.00 eq) in tetrahydrofuran (75.0 mL) was added borane dimethyl sulfide complex (10.0 M, 5.80 mL, 2.50 eq) dropwise at 0° C. under nitrogen atmosphere, the mixture was stirred at 70° C. for 2 h under nitrogen atmosphere. The reaction was quenched with methanol (30.0 mL) at 10° C. and concentrated to give a residue. The residue was purified by flash silica gel chromatography (ISCO®; 80 g SepaFlash® Silica Flash Column, Eluent of 0~50% Ethylacetate/Petroleum ether gradient @100 mL/mint) to afford 2-(2-chloro-4-nitrophenyl)ethan-1-ol, Int 1c, (4.20 g, 20.8 mmol, 90% yield) as a yellow solid. ¹H NMR (400 MHz, CDCl₃-d) δ=8.27 (d, J=2.4 Hz, 1H), 8.08 (dd, J=2.4, 8.4 Hz, 1H), 7.51 (d, J=8.4 Hz, 1H), 3.96 (t, J=6.4 Hz, 2H), 3.12 (t, J=6.4 Hz, 2H).

### Step B. Preparation of Int 1d

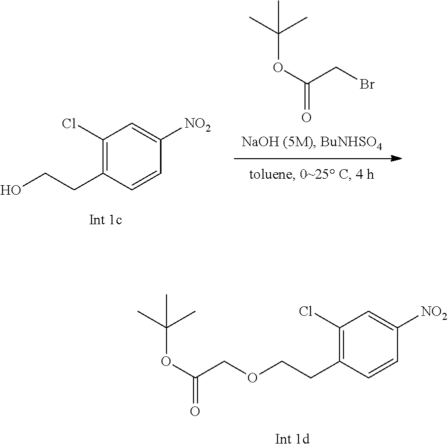

To a solution of 2-(2-chloro-4-nitrophenyl)ethan-1-ol, Int 1c, (200 mg, 992 umol, 1.00 eq) and tert-butyl 2-bromoacetate (1.50 g, 7.69 mmol, 1.14 mL, 7.75 eq) in toluene (10.0 mL) was added tetrabutylammonium hydrogensulfate (269 mg, 794 umol, 0.800 eq) at 0° C. To the above mixture was added sodium hydroxide (390 mg, 9.74 mmol, 9.82 eq) in water (2.40 mL) dropwise at 0° C. The mixture was stirred at 25° C. for 4 h. The mixture was diluted with water (10.0 mL) and extracted with ethyl acetate (3×15.0 mL). The combined organic layer was washed with brine (3×15.0 mL), dried over anhydrous sodium sulfate and filtered. The filtrate was concentrated to give a residue. The residue was purified by flash silica gel chromatography (ISCO®; 40 g SepaFlash® Silica Flash Column, Eluent of 0~16% Ethylacetate/Petroleum ether gradient @100 mL/mint) to afford tert-butyl 2-(2-chloro-4-nitrophenethoxy)acetate, Int 1d, (300 mg, 950 umol, 96% yield) as yellow oil. ¹H NMR (400 MHz, CDCl₃-d) δ=8.24 (br s, 1H), 8.18-8.01 (m, 1H), 7.60 (br d, J=7.0 Hz, 1H), 4.38-4.05 (m, 2H), 4.05-3.93 (m, 2H), 3.83 (br dd, J=1.2, 5.3 Hz, 2H), 1.48 (br dd, J=0.8, 2.8 Hz, 9H).

### Step C. Preparation of Int 1e

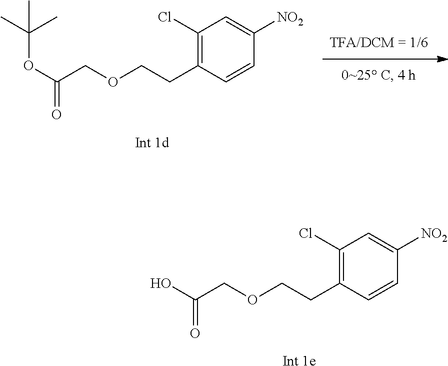 [SMILES: CC(C)(C)OC(=O)COCCc1ccc([N+](=O)[O-])cc1Cl ; "Int 1d" ; O=C(O)COCCc1ccc([N+](=O)[O-])cc1Cl ; "Int 1e"]

To a solution of tert-butyl 2-(2-chloro-4-nitrophenethoxy)acetate, Int 1d, (300 mg, 950 umol, 1.00 eq) in dichloromethane (3.00 mL) was added trifluoroacetic acid (924 mg, 8.10 mmol, 0.600 mL, 8.53 eq) at 0° C. The mixture was stirred at 25° C. for 4 h. The mixture was concentrated under reduced pressure to afford 2-(2-chloro-4-nitrophenethoxy)acetic acid, Int 1e, (240 mg, 924 umol, 97% yield) as a yellow solid. ¹H NMR (400 MHz, DMSO-d₆) δ=8.27 (d, J=2.4 Hz, 1H), 8.15 (dd, J=2.4, 8.5 Hz, 1H), 7.75 (d, J=8.6 Hz, 1H), 4.03 (s, 2H), 3.77 (t, J=6.4 Hz, 2H), 3.08 (t, J=6.4 Hz, 2H).

### Step D. Preparation of Int 1f

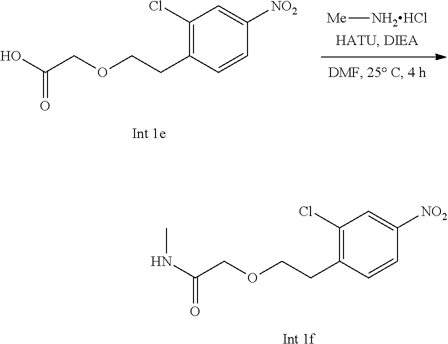

To a solution of 2-(2-chloro-4-nitrophenethoxy)acetic acid, Int 1e, (240 mg, 924 umol, 1.00 eq) and O-(7-azabenzotriazol-1-yl)-N,N,N′,N′-tetramethyluronium hexafluorophosphate (562 mg, 1.48 mmol, 1.60 eq) in dimethylformamide (5.00 mL) was added diisopropylethylamine (478 mg, 3.70 mmol, 644 uL, 4.00 eq) and methanamine;hydrochloride (74.9 mg, 1.11 mmol, 1.20 eq). The mixture was stirred at 25° C. for 4 h. The mixture was diluted with water (10.0 mL) and extracted with ethyl acetate (3×5.00 mL). The combined organic layer was washed with brine (3×15.0 mL), dried over anhydrous sodium sulfate and filtered. The filtrate was concentrated to give a residue. The residue was purified by reversed-phase HPLC (C18, 80 g; condition: water/acetonitrile=1/0-0/1, 0.1% formic acid) and lyophilized to afford 2-(2-chloro-4-nitrophenethoxy)-N-methylacetamide, Int 1f, (100 mg, 341 umol, 37% yield, 93% purity) as yellow oil. ¹H NMR (400 MHz, DMSO-d₆) δ=8.27 (d, J=2.4 Hz, 1H), 8.18-8.13 (m, 1H), 7.75 (d, J=8.6 Hz, 1H), 3.87 (s, 2H), 3.73 (t, J=6.8 Hz, 2H), 3.12 (t, J=6.6 Hz, 2H), 2.61 (d, J=4.8 Hz, 3H).

### Step E. Preparation of Int 1g

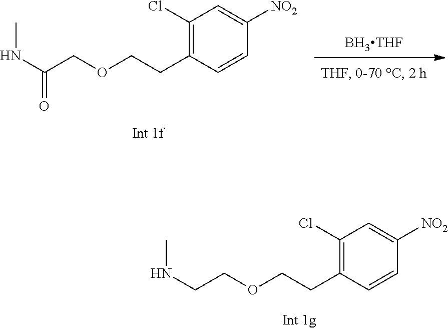 [SMILES: CNC(=O)COCCc1ccc([N+](=O)[O-])cc1Cl ; CNCCOCCc1ccc([N+](=O)[O-])cc1Cl ; "BH3·THF"]

To a solution of 2-(2-chloro-4-nitrophenethoxy)-N-methylacetamide, Int 1f, (3.00 g, 11.0 mmol, 1.00 eq) in tetrahydrofuran (30.0 mL) was added borane tetrahydrofuran (1.0 M, 55.0 mL, 5.00 eq) at 0° C. The mixture was stirred at 70° C. for 2 h. Three parallel reaction mixture was combined. The mixture was quenched with methanol (150 mL) and concentrated to give a residue. The residue was purified by flash silica gel chromatography (ISCO®; 80 g SepaFlash® Silica Flash Column, Eluent of 0~66% Ethylacetate/Petroleum ether gradient @100 mL/mint) to afford 2-(2-chloro-4-nitrophenethoxy)-N-methylethan-1-amine, Int 1g, (5.40 g, 20.9 mmol, 63% yield) as yellow oil. ¹H NMR (400 MHz, DMSO-d₆) δ=8.20 (d, J=2.0 Hz, 1H), 8.10 (dd, J=2.4, 8.5 Hz, 1H), 7.68 (d, J=8.4 Hz, 1H), 5.78 (br s, 1H), 3.67 (t, J=6.8 Hz, 2H), 3.61 (dt, J=2.0, 5.6 Hz, 2H), 3.05 (t, J=6.8 Hz, 2H), 2.77-2.56 (m, 2H), 2.29-2.22 (m, 3H).

### Step F. Preparation of Int 1h

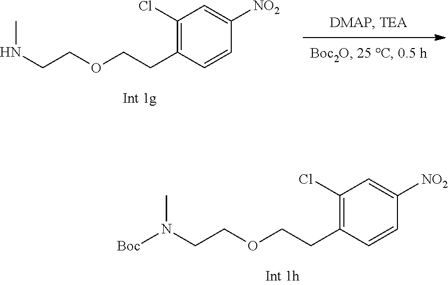 [SMILES: CNCCOCCc1ccc([N+](=O)[O-])cc1Cl ; "DMAP, TEA" ; CN(CCOCCc1ccc([N+](=O)[O-])cc1Cl)C(=O)OC(C)(C)C]

To a solution of 2-(2-chloro-4-nitrophenethoxy)-N-methylethan-1-amine, Int 1g, (5.40 g, 20.9 mmol, 1.00 eq) in di-tert-butyl dicarbonate (50.0 mL) was added triethylamine (6.54 g, 64.7 mmol, 9.00 mL, 3.10 eq) and 4-dimethylaminopyridin (510 mg, 4.17 mmol, 0.200 eq). The mixture was stirred at 25° C. for 0.5 h. The mixture was concentrated to give a residue. The residue was purified by flash silica gel chromatography (ISCO®; 120 g SepaFlash® Silica Flash Column, Eluent of 0~20% ethyl acetate/Petroleum ether gradient @100 mL/mint) to afford tert-butyl (2-(2-chloro-4-nitrophenethoxy)ethyl)(methyl)carbamate, Int 1h, (5.50 g, 15.3 mmol, 73% yield) as a yellow solid. ¹H NMR (400 MHz, DMSO-d₆) δ=8.26 (d, J=2.4 Hz, 1H), 8.13 (dd, J=2.4, 8.5 Hz, 1H), 7.67 (d, J=8.6 Hz, 1H), 3.68 (t, J=6.4 Hz, 2H), 3.48 (t, J=5.6 Hz, 2H), 3.30-3.24 (m, 2H), 3.06 (t, J=6.4 Hz, 2H), 2.73 (s, 3H), 1.36 (s, 9H).

### Step G. Preparation of Int 1i

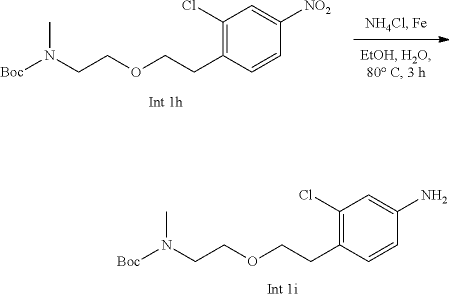

To a solution of tert-butyl (2-(2-chloro-4-nitrophenethoxy)ethyl)(methyl)carbamate, Int 1h, (6.00 g, 16.7 mmol, 1.00 eq) in ethyl alcohol (110 mL) were added saturated ammonium chloride (2.68 g, 50.2 mmol, 3.00 eq) in water (13.0 mL) and iron (4.67 g, 83.6 mmol, 5.00 eq). The mixture was stirred at 80° C. for 3 h. The mixture was diluted with water (100 mL) and extracted with ethyl acetate (3×50.0 mL). The combined organic layer was washed with brine (3×100 mL), dried over anhydrous sodium sulfate and filtered. The filtrate was concentrated to afford tert-butyl (2-(4-amino-2-chlorophenethoxy)ethyl)(methyl)carbamate, Int 1i, (5.00 g, 15.2 mmol, 91% yield) as yellow oil. ¹H NMR (400 MHz, DMSO-d₆) δ=6.96 (d, J=8.2 Hz, 1H), 6.58 (d, J=2.4 Hz, 1H), 6.44 (dd, J=2.4, 8.3 Hz, 1H), 5.28-5.14 (m, 2H), 3.54-3.42 (m, 4H), 3.33 (s, 1H), 3.31-3.25 (m, 2H), 2.80-2.75 (m, 3H), 2.75-2.68 (m, 2H), 1.39 (s, 9H).

### Step H. Preparation of Int 1j

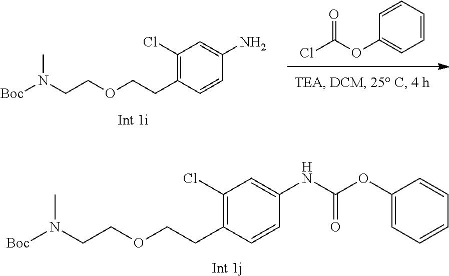

To a solution of tert-butyl (2-(4-amino-2-chlorophenethoxy)ethyl)(methyl)carbamate, Int 1i, (2.50 g, 7.60 mmol, 1.00 eq) in dichloromethane (25.0 mL) was added triethylamine (2.31 g, 22.8 mmol, 3.17 mL, 3.00 eq) and phenyl carbonochloridate (1.79 g, 11.4 mmol, 1.43 mL, 1.50 eq). The mixture was stirred at 25° C. for 4 h. The mixture was concentrated to give a residue. The residue was purified by flash silica gel chromatography (ISCO®; 60 g SepaFlash® Silica Flash Column, Eluent of 0~66% Ethylacetate/Petroleum ether gradient @100 mL/mint) to afford tert-butyl (2-(2-chloro-4-((phenoxycarbonyl)amino)phenethoxy)ethyl)(methyl) carbamate, Int 1j, (2.60 g, 5.79 mmol, 76% yield) as yellow oil. ¹H NMR (400 MHz, DMSO-d₆) δ=7.94 (d, J=2.0 Hz, 1H), 7.45-7.41 (m, 3H), 7.32-7.27 (m, 2H), 7.21-7.19 (m, 3H), 3.69-3.56 (m, 2H), 3.48 (br t, J=5.4 Hz, 2H), 3.26 (q, J=5.6 Hz, 2H), 2.92-2.72 (m, 1H), 2.76 (br d, J=2.0 Hz, 1H), 2.68 (s, 3H), 1.39-1.33 (m, 9H).

### Step I. Preparation of Int 1k

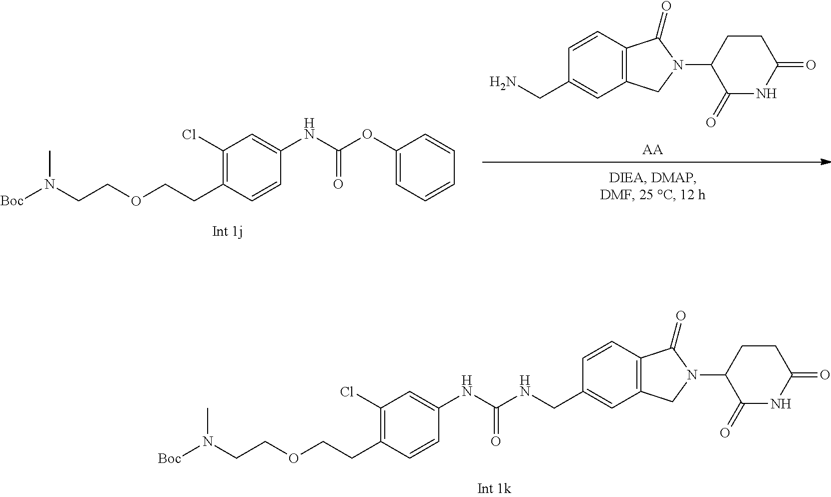 [SMILES: NCc1ccc2c(c1)CN(C1CCC(=O)NC1=O)C2=O ; CN(CCOCCc1ccc(NC(=O)Oc2ccccc2)cc1Cl)C(=O)OC(C)(C)C ; "AA" ; CN(CCOCCc1ccc(NC(=O)NCc2ccc3c(c2)CN(C2CCC(=O)NC2=O)C3=O)cc1Cl)C(=O)OC(C)(C)C]

To a solution of 3-(5-(aminomethyl)-1-oxoisoindolin-2-yl)piperidine-2,6-dione, Int 1j, (609 mg, 2.23 mmol, 1.00 eq) in N,N-dimethylformamide (10.0 ML) was added diisopropylethylamine (864 mg, 6.68 mmol, 1.16 mL, 3.00 eq), 4-dimethylaminopyridin (272 mg, 2.23 mmol, 1.00 eq) and tert-butyl (2-(2-chloro-4-((phenoxycarbonyl)amino)phenethoxy)ethyl)(methyl)carbamate, Int AA, (1.00 g, 2.23 mmol, 1.00 eq). The mixture was stirred at 25° C. for 12 h. The mixture was concentrated to give a residue. The residue was purified by reversed-phase HPLC (C18,120 g; condition: water/acetonitrile=1/0-0/1, 0.1% formic acid) and lyophilized to afford tert-butyl(2-(2-chloro-4-(3-((2-(2,6-dioxopiperidin-3-yl)-1-oxoisoindolin-5-yl)methyl)ureido) phenethoxy)ethyl)(methyl)carbamate, Int 1k, (300 mg, 444 umol, 20% yield, 93% purity) as a yellow solid. ¹H NMR (400 MHz, DMSO-d₆) δ=8.78 (s, 1H), 7.72-7.64 (m, 2H), 7.51 (s, 1H), 7.44 (d, J=7.6 Hz, 1H), 7.22-7.11 (m, 2H), 6.80 (t, J=5.6 Hz, 1H), 5.10 (dd, J=5.2, 13.3 Hz, 1H), 4.47-4.27 (m, 3H), 3.55 (br t, J=6.8 Hz, 2H), 3.47 (br t, J=5.2 Hz, 2H), 3.29-3.25 (m, 2H), 2.83 (br t, J=7.0 Hz, 2H), 2.75 (br s, 3H), 2.61 (br d, J=2.2 Hz, 1H), 2.57 (br s, 1H), 2.43-2.35 (m, 1H), 2.04-1.92 (m, 1H), 1.38 (s, 9H).

### Step J. Preparation of Compound IG-3

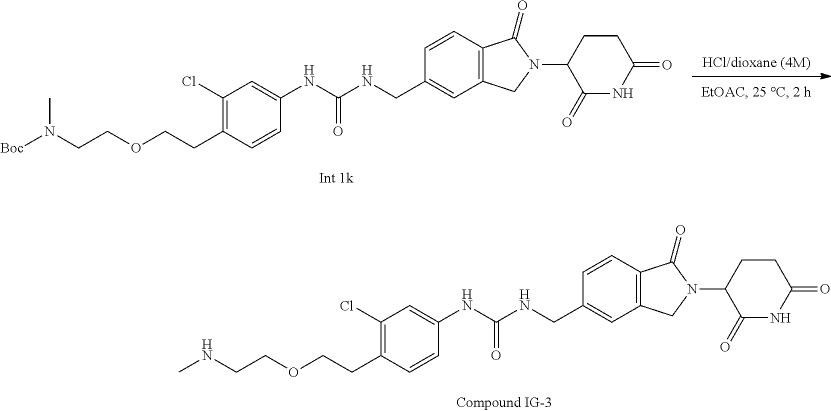 [SMILES: CN(CCOCCc1ccc(NC(=O)NCc2ccc3c(c2)CN(C2CCC(=O)NC2=O)C3=O)cc1Cl)C(=O)OC(C)(C)C ; CNCCOCCc1ccc(NC(=O)NCc2ccc3c(c2)CN(C2CCC(=O)NC2=O)C3=O)cc1Cl]

To a solution of tert-butyl (2-(2-chloro-4-(3-((2-(2,6-dioxopiperidin-3-yl)-1-oxoisoindolin-5-yl)methyl)ureido)phenethoxy)ethyl)(methyl)carbamate, Int 1k, (270 mg, 430 umol, 1.00 eq) in ethyl acetate (3.00 mL) was added hydrochloric acid/dioxane (4.00 M, 2.16 mL, 20.1 eq). The mixture was stirred at 25° C. for 2 h. The mixture was concentrated under reduced pressure to give a residue. The residue was triturated with ethyl acetate (10.0 mL) and filtered to afford 1-(3-chloro-4-(2-(2-(methylamino)ethoxy)ethyl)phenyl)-3-((2-(2,6-dioxopiperidin-3-yl)-1-oxoisoindolin-5-yl)methyl)urea, Compound IG-3, (81.8 mg, 153 umol, 36% yield, 99% purity, hydrochloride) as a yellow solid. ¹H NMR (400 MHz, DMSO-d₆) δ=10.98 (s, 1H), 9.12 (s, 1H), 8.61 (br s, 2H), 7.79-7.62 (m, 2H), 7.52 (s, 1H), 7.44 (br d, J=7.6 Hz, 1H), 7.31-7.11 (m, 2H), 7.02 (br t, J=5.6 Hz, 1H), 5.11 (br dd, J=4.8, 13.0 Hz, 1H), 4.50-4.37 (m, 3H), 4.35-4.28 (m, 1H), 3.66 (br t, J=4.8 Hz, 2H), 3.62 (br t, J=7.0 Hz, 2H), 3.08 (br s, 2H), 2.93-2.86 (m, 3H), 2.62 (br s, 1H), 2.54 (br s, 3H), 2.40 (br s, 1H), 2.01 (br dd, J=5.6, 10.7 Hz, 1H). MS (ESI) m/z 528.5 [M+H]⁺

The compounds in Table 4 were prepared in a manner similar to that described for Compound IG-3 using Int AA and the appropriately substituted phenyl carbamate.

TABLE 4

| IG No. | Structure | ¹H NMR/MS |
|---|---|---|
| IG-5 | 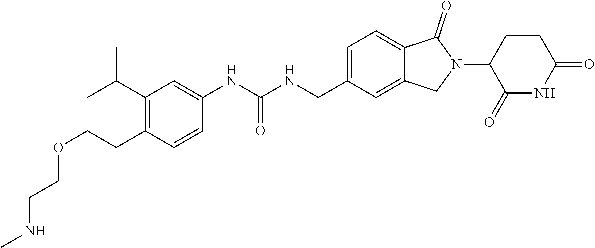 [SMILES: CNCCOCCc1ccc(NC(=O)NCc2ccc3c(c2)CN(C2CCC(=O)NC2=O)C3=O)cc1C(C)C] (2-(2,6-dioxopiperidin-3-yl)-1-oxoisoindolin-5-yl)methyl (3-chloro- 4-(2-(2-(methylamino)ethoxy)ethyl)phenyl)carbamate | ¹H NMR (400 MHz, DMSO-d₆) δ = 10.97 (s, 1H), 8.98-8.70 (m, 3H), 7.68 (d, J = 7.8 Hz, 1H), 7.51 (s, 1H), 7.44 (br d, J = 7.9 Hz, 1H), 7.30 (d, J = 1.7 Hz, 1H), 7.19 (dd, J = 1.7, 8.3 Hz, 1H), 7.02 (d, J = 8.3 Hz, 2H), 5.10 (dd, J = 5.1, 13.3 Hz, 1H), 4.47 (br s, 1H), 4.35-4.28 (m, 3H), 3.66 (br t, J = 5.1 Hz, 2H), 3.54 (br t, J = 7.4 Hz, 2H), 3.12-3.04 (m, 3H), 2.95-2.88 (m, 1H), 2.81 (br t, J = 7.4 Hz, 2H), 2.60-2.54 (m, 4H), 2.40 (br d, J = 4.4 Hz, 1H), 2.02-1.96 (m, 1H), 1.15 (d, J = 6.7 Hz, 6H). MS (ESI) m/z 536.3 [M + H]⁺ |
| IG-6 | 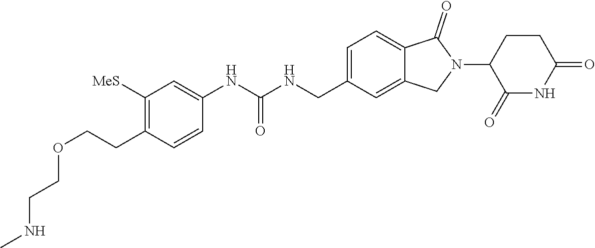 [SMILES: CNCCOCCc1ccc(NC(=O)NCc2ccc3c(c2)CN(C2CCC(=O)NC2=O)C3=O)cc1SC] 1-((2-(2,6-dioxopiperidin-3-yl)-1-oxoisoindolin-5-yl)methyl)-3-(4- (2-(2-(methylamino)ethoxy)ethyl)-3-(methylthio)phenyl)urea | ¹H NMR (400 MHz, DMSO-d₆) δ = 10.97 (s, 1H), 9.03 (s, 1H), 8.75 (br s, 2H), 7.69 (d, J = 7.8 Hz, 1H), 7.51 (s, 1H), 7.48-7.39 (m, 2H), 7.16-7.04 (m, 2H), 7.01 (br t, J = 5.5 Hz, 1H), 5.10 (dd, J = 5.0, 13.3 Hz, 1H), 4.50-4.36 (m, 3H), 4.35-4.26 (m, 1H), 3.66 (t, J = 5.1 Hz, 2H), 3.59 (t, J = 7.3 Hz, 2H), 3.13-3.01 (m, 2H), 2.97-2.80 (m, 3H), 2.59 (br d, J = 17.9 Hz, 1H), 2.53 (br s, 3H), 2.44-2.35 (m, 4H), 2.07-1.94 (m, 1H). MS (ESI) m/z 540.3 [M + H]⁺ |
| IG-7 | 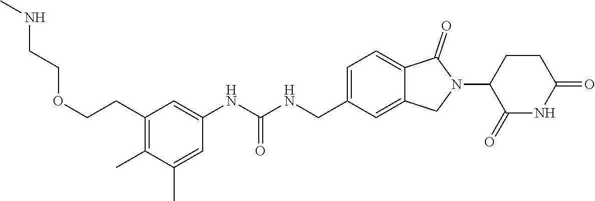 [SMILES: CNCCOCCc1cc(NC(=O)NCc2ccc3c(c2)CN(C2CCC(=O)NC2=O)C3=O)cc(C)c1C] 1-((2-(2,6-dioxopiperidin-3-yl)-1-oxoisoindolin-5-yl)methyl)-3-(3- (2-((2-methoxyethyl)amino)ethyl)-4,5-dimethylphenyl)urea | ¹H NMR (400 MHz, DMSO-d₆) δ = 10.97 (s, 1H), 8.66-8.39 (m, 2H), 7.69 (d, J = 8.0 Hz, 1H), 7.50 (s, 1H), 7.43 (d, J = 7.6 Hz, 1H), 7.19 (s, 1H), 6.99 (s, 1H), 6.88-6.75 (m, 1H), 5.17-5.04 (m, 1H), 4.53-4.22 (m, 4H), 3.65 (t, J = 5.2 Hz, 2H), 3.56 (t, J = 7.4 Hz, 2H), 3.09 (br s, 2H), 2.98-2.86 (m, 1H), 2.81 (br t, J = 7.2 Hz, 2H), 2.63-2.57 (m, 1H), 2.53 (br d, J = 4.8 Hz, 3H), 2.38 (br dd, J = 4.4, 13.2 Hz, 1H), 2.17 (s, 3H), 2.09 (s, 3H), 2.04- 1.96 (m, 1H) MS (ESI) m/z 522.3 [M + H]⁺ |
| IG-12 | 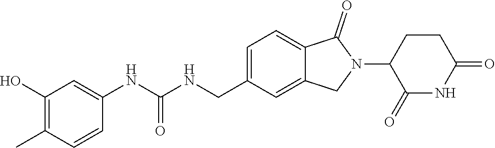 [SMILES: Cc1ccc(NC(=O)NCc2ccc3c(c2)CN(C2CCC(=O)NC2=O)C3=O)cc1O] 1-((2-(2,6-dioxopiperidin-3-yl)-1-oxoisoindolin-5-yl)methyl)-3-(3- hydroxy-4-methylphenyl)urea | ¹H NMR (400 MHz, DMSO-d₆) δ = 10.97 (br d, J = 2.4 Hz, 1H), 9.13 (s, 1H), 8.50-8.38 (m, 1H), 7.69 (d, J = 7.6 Hz, 1H), 7.51 (s, 1H), 7.44 (d, J = 8.0 Hz, 1H), 7.04 (d, J = 2.0 Hz, 1H), 6.85 (d, J = 8.4 Hz, 1H), 6.71-6.61 (m, 2H), 5.10 (dd, J = 5.1, 13.2 Hz, 1H), 4.50-4.25 (m, 4H), 2.91 (ddd, J = 5.4, 13.6, 17.6 Hz, 1H), 2.68-2.57 (m, 1H), 2.41-2.31 (m, 1H), 2.07 (s, 1H), 2.01 (s, 3H), 2.00-1.93 (m, 1H). MS (ESI) m/z 423.2 [M + H]⁺ |
| IG-14 | 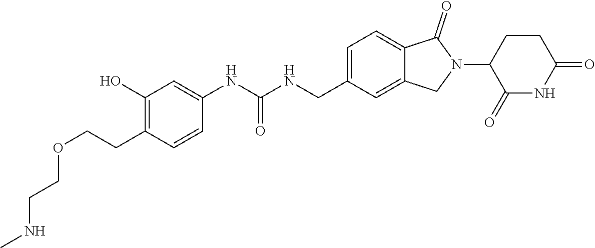 [SMILES: CNCCOCCc1ccc(NC(=O)NCc2ccc3c(c2)CN(C2CCC(=O)NC2=O)C3=O)cc1O] 1-((2-(2,6-dioxopiperidin-3-yl)-1-oxoisoindolin-5-yl)methyl)-3-(3- hydroxy-4-(2-(2-(methylamino)ethoxy)ethyl)phenyl)urea | ¹H NMR (400 MHz, DMSO-d₆) δ = 11.01 (s, 1H), 8.62 (br s, 1H), 8.30 (s, 1H), 7.69 (d, J = 7.8 Hz, 1H), 7.50 (s, 1H), 7.43 (d, J = 8.0 Hz, 1H), 7.06 (d, J = 1.6 Hz, 1H), 6.90 (d, J = 8.2 Hz, 1H), 6.86- 6.79 (m, 1H), 6.72-6.65 (m, 1H), 5.10 (dd, J = 5.0, 13.3 Hz, 1H), 4.53-4.24 (m, 4H), 3.56-3.49 (m, 5H), 2.87 (br t, J = 5.2 Hz, 2H), 2.68 (br d, J = 7.2 Hz, 2H), 2.64-2.53 (m, 1H), 2.43 (s, 3H), 2.38 (br dd, J = 4.6, 13.2 Hz, 2H), 2.03-1.96 (m, 1H). MS (ESI) m/z 510.1 [M + H]⁺ |
| IG-32 | 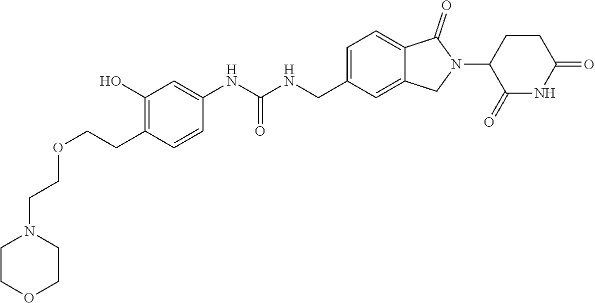 [SMILES: O=C1CCC(N2Cc3cc(CNC(=O)Nc4ccc(CCOCCN5CCOCC5)c(O)c4)ccc3C2=O)C(=O)N1] 1-((2-(2,6-dioxopiperidin-3-yl)-1-oxoisoindolin-5-yl)methyl)-3-(3- hydroxy-4-(2-(2-morpholinoethoxy)ethyl)phenyl)urea | ¹H NMR (400 MHz, DMSO-d₆) δ = 10.97 (s, 1H), 8.43 (s, 1H), 8.15 (s, 1H), 7.69 (d, J = 7.8 Hz, 1H), 7.50 (s, 1H), 7.43 (d, J = 8.0 Hz, 1H), 7.05 (d, J = 2.0 Hz, 1H), 6.89 (d, J = 8.0 Hz, 1H), 6.69- 6.56 (m, 2H), 5.10 (dd, J = 5.2, 13.3 Hz, 1H), 4.48-4.27 (m, 4H), 3.55-3.52 (m, 4H), 3.49-3.45 (m, 4H), 2.96-2.85 (m, 1H), 2.65 (br t, J = 7.2 Hz, 2H), 2.63-2.56 (m, 1H), 2.52 (br s, 1H), 2.45 (t, J = 5.6 Hz, 2H), 2.40-2.35 (m, 4H), 2.05-1.93 (m, 1H). MS (ESI) m/z 566.2 [M + H]⁺ |
| IG-54 | 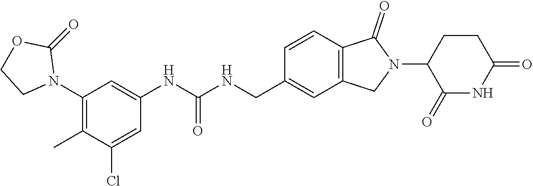 [SMILES: Cc1c(Cl)cc(NC(=O)NCc2ccc3c(c2)CN(C2CCC(=O)NC2=O)C3=O)cc1N1CCOC1=O] 1-(3-chloro-4-methyl-5-(2-oxooxazolidin-3-yl)phenyl)-3-((2-(2,6- dioxopiperidin-3-yl)-1-oxoisoindolin-5-yl)methyl)urea | ¹H NMR (400 MHz, DMSO-d₆) δ = 10.94 (br s, 1H), 9.23 (s, 1H), 8.50 (s, 1H), 7.69 (d, J = 7.6 Hz, 1H), 7.61 (d, J = 1.6 Hz, 1H), 7.51 (s, 1H), 7.44 (d, J = 8.0 Hz, 1H), 7.36 (d, J = 1.6 Hz, 1H), 7.26 (br s, 1H), 5.10 (dd, J = 5.2, 13.2 Hz, 1H), 4.49-4.28 (m, 6H), 3.89 (t, J = 7.6 Hz, 2H), 2.97-2.85 (m, 1H), 2.63-2.57 (m, 1H), 2.43-2.35 (m, 1H), 2.14 (s, 3H), 2.03-1.96 (m, 1H). MS (ESI) m/z 526.2 [M + H]⁺ |

### Example 3 Synthesis of 1-(3-chloro-4-methylphenyl)-3-((2-(2,6-dioxopiperidin-3-yl)-4-hydroxy-1-oxoisoindolin-5-yl)methyl)urea (Compound IG-15)

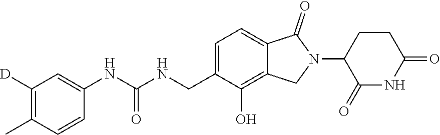 [SMILES: [2H]c1cc(NC(=O)NCc2ccc3c(c2O)CN(C2CCC(=O)NC2=O)C3=O)ccc1C]

### Step A. Preparation of Int 1l

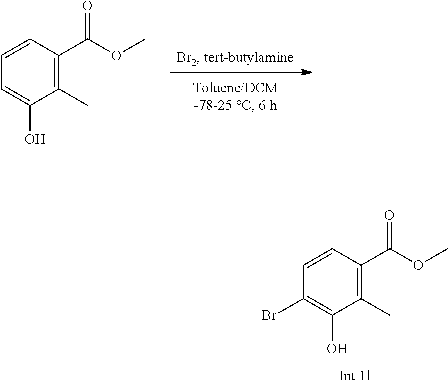

To a solution of 2-methylpropan-2-amine (4.18 g, 57.2 mmol, 6.01 mL, 1.90 eq) in toluene (50 mL) was added a mixture of dibromine (4.57 g, 28.6 mmol, 1.47 mL, 0.95 eq) in dichloromethane (10 mL) at −78° C. for 1 h, then methyl 3-hydroxy-2-methylbenzoate (5.00 g, 30.1 mmol, 1.00 eq) in dichloromethane (50 mL) was added dropwise, the mixture was stirred at 25° C. for 5 h. The mixture was concentrated to give a residue. The residue was purified by column chromatography (SiO₂, petroleum ether/ethyl acetate=10/1) to afford methyl 4-bromo-3-hydroxy-2-methylbenzoate, Int 11, (5.50 g, 22.4 mmol, 75% yield) as a white solid. ¹H NMR (400 MHz, DMSO-d₆) δ=9.63-9.16 (m, 1H), 7.46 (d, J=8.4 Hz, 1H), 7.17 (d, J=8.4 Hz, 1H), 3.80 (s, 3H), 2.38 (s, 3H)

### Step B. Preparation of Int 1m

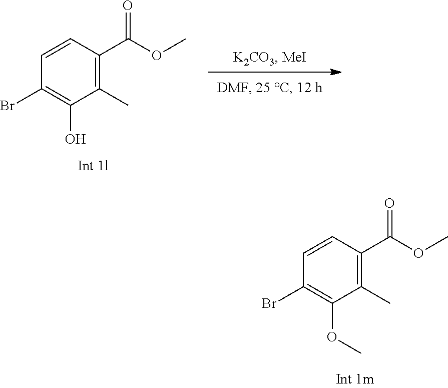

To a solution of methyl 4-bromo-3-hydroxy-2-methylbenzoate, Int 11, (5.50 g, 22.4 mmol, 1.00 eq) in NN-dimethylformamide (30 mL) was added potassium carbonate (6.20 g, 44.9 mmol, 2.00 eq) and methyl iodide (9.56 g, 67.3 mmol, 4.19 mL, 3.00 eq), the mixture was stirred at 25° C. for 12 h. The mixture was diluted with water (30 mL) and extracted with ethyl acetate (3×30 mL). The combined organic layers were washed with brine (2×20 mL), dried over sodium sulfate and filtered. The filtrate was concentrated under reduced pressure to give a residue. The residue was purified by column chromatography (SiO₂, petroleum ether/ethyl acetate=3/1) to afford methyl 4-bromo-3-methoxy-2-methylbenzoate, Int 1m, (5.70 g, 22.0 mmol, 98% yield) as yellow oil, ¹H NMR (400 MHz, CDCl₃-d) δ=7.56-7.49 (m, 1H), 7.47-7.41 (m, 1H), 3.89 (s, 3H), 3.81 (s, 3H), 2.56 (s, 3H).

### Step C. Preparation of Int 1n

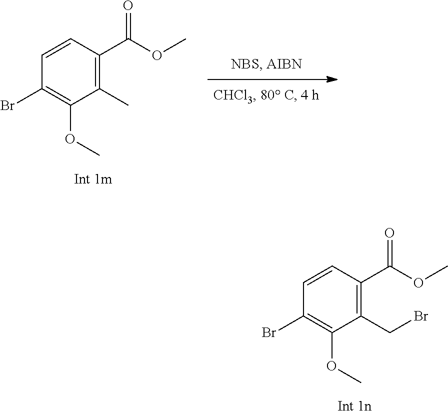

To a solution of methyl 4-bromo-3-methoxy-2-methylbenzoate, Int 1m, (5.70 g, 22.0 mmol, 1.00 eq) and N-bromosuccinimide (4.31 g, 24.2 mmol, 1.10 eq) in trichloromethane (2.00 mL) was added (E)-3,3′-(diazene-1,2-diyl)bis(2-methylpropanenitrile) (361 mg, 2.20 mmol, 0.10 eq), the mixture was stirred at 80° C. for 4 h. The mixture was filtered and the filtrate was concentrated to give a residue. The residue was purified by column chromatography (SiO₂, petroleum ether/ethyl acetate=10/1) to afford methyl 4-bromo-2-(bromomethyl)-3-methoxybenzoate, Int 1n, (6.50 g, 19.2 mmol, 87% yield) as yellow oil. ¹H NMR (400 MHz, CDCl₃) δ=7.67-7.58 (m, 2H), 5.09 (s, 2H), 4.03 (s, 3H), 3.95 (s, 3H).

### Step D. Preparation of Int 1o

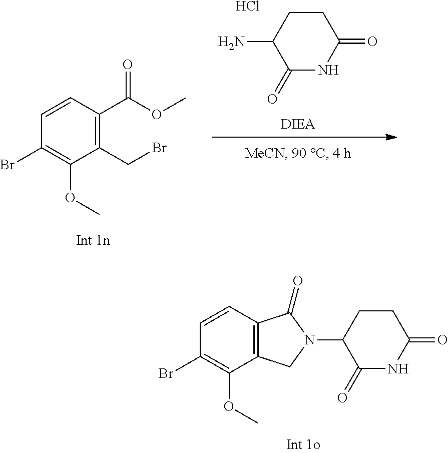

To a solution of methyl 4-bromo-2-(bromomethyl)-3-methoxybenzoate, Int 1n, (6.50 g, 19.2 mmol, 1.00 eq) and 3-aminopiperidine-2,6-dione (4.75 g, 28.9 mmol, 1.50 eq, hydrochloride) in acetonitrile (60 mL) was added diisopropylethylamine (7.46 g, 57.7 mmol, 10.1 mL, 3.00 eq), the mixture was stirred at 90° C. for 4 h. The mixture was triturated with ethyl acetate/water-2/1 (50 mL) and filtered, the filter cake was concentrated under reduced pressure to afford 3-(5-bromo-4-methoxy-1-oxoisoindolin-2-yl)piperidine-2,6-dione, Int to, (5.00 g, 14.2 mmol, 74% yield) as a brown solid. ¹H NMR (400 MHz, DMSO-d₆) δ=11.01 (s, 1H), 7.76 (d, J=8.0 Hz, 1H), 7.36 (d, J=8.0 Hz, 1H), 5.11 (dd, J=5.2, 13.6 Hz, 1H), 4.81-4.67 (m, 1H), 4.62-4.50 (m, 1H), 3.99 (s, 3H), 2.99-2.85 (m, 1H), 2.66-2.56 (m, 1H), 2.47-2.38 (m, 1H), 2.01 (tdd, J=2.8, 5.6, 12.6 Hz, 1H).

### Step E. Preparation of Int 1p

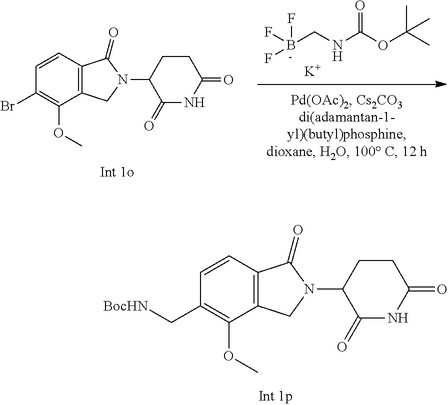

To a solution of 3-(5-bromo-4-methoxy-1-oxoisoindolin-2-yl)piperidine-2,6-dione, Int to, (1.00 g, 2.83 mmol, 1.00 eq) and (((tert-butoxycarbonyl)amino)methyl)trifluoroborate (873 mg, 3.68 mmol, 1.30 eq) in dioxane (10 mL) and water (1.00 mL) was added palladium(II) acetate (127 mg, 566 umol, 0.20 eq), cesium carbonate (1.85 g, 5.66 mmol, 2.00 eq) and di(adamantan-1-yl)(butyl)phosphane (406 mg, 1.13 mmol, 0.40 eq), the mixture was stirred at 100° C. for 12 h under nitrogen atmosphere. The mixture was filtered and the filtrate was concentrated to give a residue. The residue was purified by column chromatography (SiO₂, petroleum ether/ethyl acetate=3/1) to afford tert-butyl ((2-(2,6-dioxopiperidin-3-yl)-4-methoxy-1-oxoisoindolin-5-yl)methyl)carbamate, Int 1p, (500 mg, 1.24 mmol, 44% yield) as a yellow solid. ¹H NMR (400 MHz, DMSO-d₆) δ=11.00 (s, 1H), 7.43-7.38 (m, 1H), 7.38-7.29 (m, 2H), 5.11 (dd, J=5.2, 13.6 Hz, 1H), 4.68 (d, J=17.2 Hz, 1H), 4.50 (d, J=16.8 Hz, 1H), 4.20 (br d, J=5.9 Hz, 2H), 3.94 (s, 3H), 2.97-2.87 (m, 1H), 2.57 (br s, 1H), 2.36 (br s, 1H), 2.01 (br dd, J=2.4, 5.6 Hz, 1H), 1.40 (s, 9H). MS (ESI) m/z 404.2 [M+H]⁺

### Step F. Preparation of Int BB

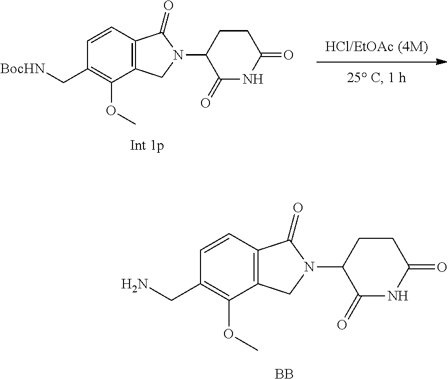

To a solution of tert-butyl ((2-(2,6-dioxopiperidin-3-yl)-4-methoxy-1-oxoisoindolin-5-yl)methyl)carbamate, Int 1p, (500 mg, 1.24 mmol, 1.00 eq) in ethyl acetate (10 mL) was added hydrochloric acid/ethyl acetate (4 M, 16.7 mL, 53.8 eq), the mixture was stirred at 25° C. for 1 h. The mixture was concentrated to afford 3-(5-(aminomethyl)-4-methoxy-1-oxoisoindolin-2-yl)piperidine-2,6-dione, Int BB, (250 mg, 824 umol, 66% yield) as a yellow solid.

### Step G. Preparation of Int 1q

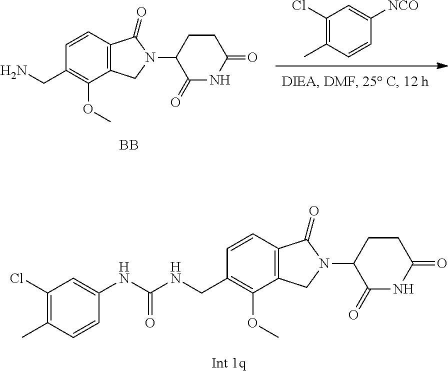

To a solution of 3-(5-(aminomethyl)-4-methoxy-1-oxoisoindolin-2-yl)piperidine-2,6-dione, Int BB, (150 mg, 495 umol, 1.00 eq) and 2-chloro-4-isocyanato-1-methylbenzene (166 mg, 989 umol, 2.00 eq) in N,N-dimethylformamide (2.00 mL) was added diisopropylethylamine (150 mg, 1.16 mmol, 202 uL, 2.35 eq), the mixture was stirred at 25° C. for 12 h. The mixture was diluted with water (2.00 mL), acetonitrile (2.00 mL) and filtered. The filtrate was purified by reversed-phase (C18, 12 g; condition: water/acetonitrile=1/0-0/1, 0.1% formic acid) and lyophilized to afford 1-(3-chloro-4-methylphenyl)-3-((2-(2,6-dioxopiperidin-3-yl)-4-methoxy-1-oxoisoindolin-5-yl)methyl)urea, Int 1q, (60.0 mg, 127 umol, 26% yield) as a white solid. MS (ESI) m/z 471.1 [M+H]⁺

### Step G. Preparation of Compound IG-15

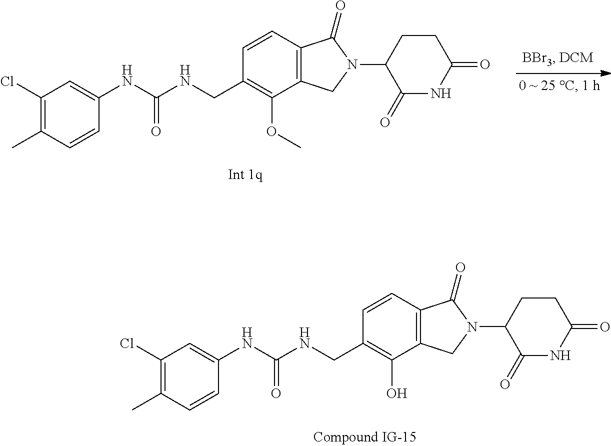 [SMILES: COc1c(CNC(=O)Nc2ccc(C)c(Cl)c2)ccc2c1CN(C1CCC(=O)NC1=O)C2=O ; Cc1ccc(NC(=O)NCc2ccc3c(c2O)CN(C2CCC(=O)NC2=O)C3=O)cc1Cl]

To a solution of 1-(3-chloro-4-methylphenyl)-3-((2-(2,6-dioxopiperidin-3-yl)-4-methoxy-1-oxoisoindolin-5-yl)methyl)urea, Int 1q, (60.0 mg, 127 umol, 1.00 eq) in dichloromethane (5.00 mL) was added tribromoborane (319 mg, 1.27 mmol, 123 uL, 10.0 eq) at 0° C., the mixture was stirred at 25° C. for 1 h. The mixture was quenched with water (5.00 mL) and concentrated under reduced pressure to removed dichloromethane (5.00 mL) to give a residue. The residue was purified by reversed-phase HPLC (C18, 12 g; condition: water/acetonitrile=1/0-0/1, 0.1% formic acid) and lyophilized to afford 1-(3-chloro-4-methylphenyl)-3-((2-(2,6-dioxopiperidin-3-yl)-4-hydroxy-1-oxoisoindolin-5-yl)methyl)urea, Compound IG-15, (27.72 mg, 59.5 umol, 47% yield, 98% purity) as a white solid. ¹H NMR (400 MHz, DMSO-d₆) δ=10.98 (s, 1H), 10.05 (s, 1H), 8.82 (s, 1H), 7.62 (d, J=1.6 Hz, 1H), 7.35 (d, J=7.6 Hz, 1H), 7.24-7.16 (m, 2H), 7.14-7.08 (m, 1H), 6.77 (br t, J=5.8 Hz, 1H), 5.09 (dd, J=5.0, 13.2 Hz, 1H), 4.41-4.19 (m, 4H), 2.97-2.84 (m, 1H), 2.60 (br d, J=18.2 Hz, 1H), 2.42-2.35 (m, 1H), 2.23 (s, 3H), 2.05-1.95 (m, 1H). MS (ESI) m/z 445.2[M+H]⁺

The compounds in Table 5 were prepared in a manner similar to that described for Compound IG-15 using Int BB and the appropriately substituted carboxylic acid, aryl isocyanate, or phenyl carbamate (cf. Example 2).

TABLE 5

| IG No. | Structure | ¹H NMR/MS |
|---|---|---|
| IG-25 | 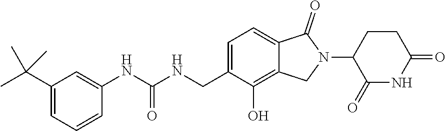 [SMILES: CC(C)(C)c1cccc(NC(=O)NCc2ccc3c(c2O)CN(C2CCC(=O)NC2=O)C3=O)c1] | ¹H NMR (400 MHz, DMSO-d₆) δ = 10.98 (s, 1H), 10.19 (br s, 1H), 8.73 (s, 1H), 7.38-7.34 (m, 2H), 7.25-7.22 (m, 1H), 7.20 (d, J = 7.6 Hz, 1H), 7.15 (t, J = 7.9 Hz, 1H), 6.96 (d, J = 7.9 Hz, 1H), 6.74 (br t, J = 6.0 Hz, 1H), 5.09 (dd, J = 5.1, 13.3 Hz, 1H), 4.38- 4.35 (m, 1H), 4.34 (s, 2H), 4.27-4.20 (m, 1H), 2.96- 2.85 (m, 1H), 2.62-2.56 (m, 1H), 2.38 (br dd, J = 4.4, 12.9 Hz, 1H), 2.04-1.97 (m, 1H), 1.25 (s, 9H). |
| | 1-(3-(tert-butyl)phenyl)-3-((2-(2,6-dioxopiperidin-3-yl)-4-hydroxy-1- | MS (ESI) m/z 465.4 [M + H]⁺ |
| | oxoisoindolin-5-y1)methyl)urea | |
| IG-26 | 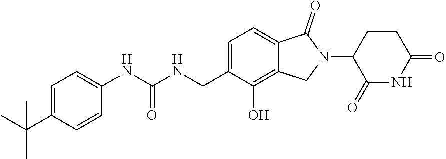 [SMILES: CC(C)(C)c1ccc(NC(=O)NCc2ccc3c(c2O)CN(C2CCC(=O)NC2=O)C3=O)cc1] | |
| | 1-(4-(tert-butyl)phenyl)-3-((2-(2,6-dioxopiperidin-3-yl)-4-hydroxy-1- | |
| | oxoisoindolin-5-y1)methyl)urea | |
| IG-27 | 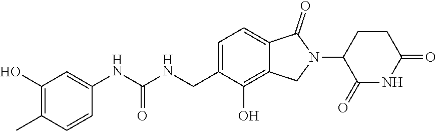 [SMILES: Cc1ccc(NC(=O)NCc2ccc3c(c2O)CN(C2CCC(=O)NC2=O)C3=O)cc1O] | ¹H NMR (400 MHz, DMSO-d₆) δ = 10.98 (s, 1H), 10.25 (s, 1H), 9.16 (s, 1H), 8.53 (s, IH), 7.35 (d, J = 8.0 Hz, 1H), 7.19 (d, J = 7.6 Hz, 1H), 7.00 (s, 1H), 6.87 (d, J = 7.6 Hz, 1H), 6.67 (s, 1H), 6.63-6.59 (m, 1H), 5.09 (dd, J = 5.2, 13.2 Hz, 1H), 4.40-4.18 (m, 4H), 2.91 (br s, 1H), 2.62 (br d, J = 2.8 Hz, 1H), 2.36 (br d, J = 4.4 Hz, 1H), 2.01 (s, 4H). MS (ESI) m/z 439.2 [M + H]⁺ |
| | 1-((2-(2,6-dioxopiperidin-3-yl)-4-hydroxy-1-oxoisoindolin-5-yl)methyl)-3-(3- | |
| | hydroxy-4-methylphenyl)urea | |
| IG-28 | 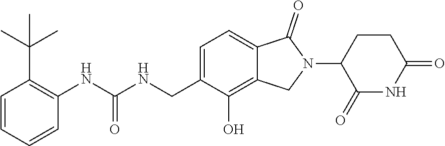 [SMILES: CC(C)(C)c1ccccc1NC(=O)NCc1ccc2c(c1O)CN(C1CCC(=O)NC1=O)C2=O] | ¹H NMR (400 MHz, DMSO-d₆) δ = 11.07-10.85 (m, 1H), 10.61-10.33 (m, 1H), 7.69 (s, 1H), 7.35 (dd, J = 3.8, 7.2 Hz, 2H), 7.20-7.16 (m, 3H), 7.15-7.09 (m, 2H), 5.09 (dd, J = 5.2, 13.4 Hz, 1H), 4.37-4.29 (m, 3H), 4.24-4.14 (m, 1H), 2.97-2.85 (m, 1H), 2.63- 2.58 (m, 1H), 2.46-2.40 (m, 1H), 2.03-1.97 (m, 1H), 1.31 (s, 9H). MS (ESI) m/z 465.4 [M + H]⁺ |
| | 1-(2-(tert-butyl)phenyl)-3-((2-(2,6-dioxopiperidin-3-yl)-4-hydroxy-1 - | |
| | oxoisoindolin-5-yl)methyl)urea | |
| IG-37 | 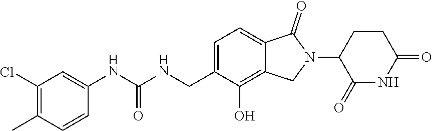 | ¹H NMR (400 MHz, DMSO-d₆) δ = 11.01 (s, 1H), 9.90 (s, 1H), 9.41 (t, J = 5.6 Hz, 1H), 7.99 (d, J = 1.6 Hz, 1H), 7.88 (dd, J = 1.6, 8.0 Hz, 1H), 7.59 (d, J = 8.0 Hz, 1H), 7.36 (d, J = 7.6 Hz, 1H), 7.23 (d, J = 7.6 Hz, 1H), 5.11 (dd, J = 4.8, 13.2 Hz, 1H), 4.52 (d, J = 6.0 Hz, 2H), 4.42-4.34 (m, 1H), 4.31-4.22 (m, 1H), 3.02-2.84 (m, 1H), 2.64-2.58 (m, 1H), 2.43 (s, 3H), 2.40-2.35 (m, 1H), 2.10-1.97 (m, 1H). MS (ESI) m/z 470.1 [M + H]⁺ |
| | 2-(3-chloro-4-methylphenyl)-N-((2-(2,6-dioxopiperidin-3-yl)-4-hydroxy-1- | |
| | oxoisoindolin-5-y1)methyl)-2-oxoacetamide | |
| IG-38 | 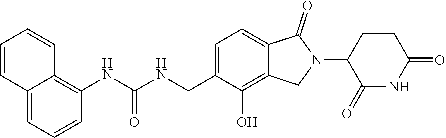 [SMILES: O=C1CCC(N2Cc3c(ccc(CNC(=O)Nc4cccc5ccccc45)c3O)C2=O)C(=O)N1] | ¹H NMR (400 MHz, DMSO-d6) δ = 10.98 (br s, 1H), 10.22 (br s, IH), 8.78 (s, 1H), 8.07 (br d, J = 8.0 Hz, 1H), 7.92 (1, J = 7.6 Hz, 2H), 7.60 (d, J = 8.0 Hz, 1H), 7.57-7.49 (m, 2H), 7.47-7.39 (m, 2H), 7.24-7.15 (m, 2H), 5.10 (dd, J = 4.8, 13.3 Hz, 1H), 4.41 (br d, J = 6.0 Hz, 2H), 4.39-4.34 (m, 1H), 4.28-4.21 (m, 1H), 2.98-2.85 (m, 1H), 2.60 (br d, J = 16.0 Hz, 1H), 2.38 (dq, J = 4.4, 13.2 Hz, 1H), 2.06 - 1.97 (m, 1H). MS (ESI) m/7. 459.2 [M + H]⁺ |
| | 1-((2-(2,6-dioxopiperidin-3-yl)-4-hydroxy-1-oxoisoindolin-5-yl)methyl)-3- | |
| | (naphthalen-1-yl)urea | |
| IG-39 | 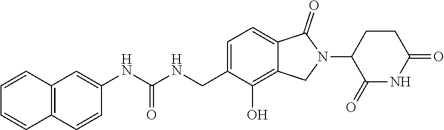 [SMILES: O=C1CCC(N2Cc3c(ccc(CNC(=O)Nc4ccc5ccccc5c4)c3O)C2=O)C(=O)N1] | ¹H NMR (400 MHz, DMSO-d₆) δ = 10.98 (s, 1H), 10.13 (s, 1H), 8.96 (s, 1H), 8.01 (s, 1H), 7.82-7.72 (m, 3H), 7.45-7.38 (m, 3H), 7.35-7.30 (m, 1H), 7.21 (d, J = 7.6 Hz, 1H), 6.85 (br t, J = 6.3 Hz, 1H), 5.13-5.06 (m, 1H), 4.42-4.33 (m, 3H), 4.29-4.21 (m, 1H), 2.95-2.85 (m, 1H), 2.62 (br d, J = 1.3 Hz, 1H), 2.40-2.34 (m, 1H), 2.04-1.98 (m, 1H). MS (ESI) m/z 459.3 [M + H]⁺ |
| | 1-((2-(2,6-dioxopiperidin-3-yl)-4-hydroxy-1-oxoisoindolin-5-yl)methyl)-3- | |
| | (naphthalen-2-yl)urea | |
| IG-40 | 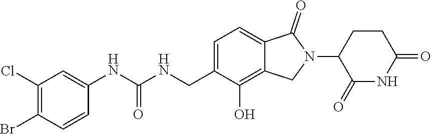 [SMILES: O=C1CCC(N2Cc3c(ccc(CNC(=O)Nc4ccc(Br)c(Cl)c4)c3O)C2=O)C(=O)N1] | ¹H NMR (400 MHz, DMSO-d₆) δ = 10.98 (br s, 1H), 10.12-9.89 (m, 1H), 9.14 (br s, 1H), 7.83 (d, J = 2.4 Hz, 1H), 7.58 (d, J = 8.8 Hz, 1H), 7.34 (d, J = 8.0 Hz, 1H), 7.23-7.17 (m, 2H), 6.94 (br t, J = 6.0 Hz, 1H), 5.10 (dd, J = 5.2, 13.2 Hz, 1H), 4.40-4.30 (m, 3H), 4.28-4.19 (m, IH), 2.97-2.83 (m, 1H), 2.60 (br d, J = 17.2 Hz, 1H), 2.36 (dt, J = 8.8, 13.0 Hz, 1H), 2.06- 1.95 (m, 1H). MS (ESI) m/z 522.9 [M + H]⁺ |
| | 1-(4-bromo-3-chlorophenyl)-3-((2-(2,6-dioxopiperidin-3-yl)-4-hydroxy-1- | |
| | oxoisoindolin-5-yl)methyl)urea | |
| IG-41 | 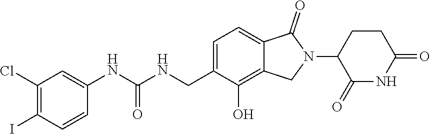 [SMILES: O=C1CCC(N2Cc3c(ccc(CNC(=O)Nc4ccc(I)c(Cl)c4)c3O)C2=O)C(=O)N1] | ¹H NMR (400 MHz, DMSO-d₆) δ = 10.98 (s, 1H), 9.98 (br s, 1H), 9.04 (br s, 1H), 7.80 (d, J = 2.4 Hz, 1H), 7.73 (d, J = 8.6 Hz, 1H), 7.34 (d, J = 7.6 Hz, 1H), 7.20 (d, J = 7.6 Hz, 1H), 7.03 (dd, J = 2.4, 8.6 Hz, 1H), 6.85 (br t, J = 5.6 Hz, IH), 5.09 (dd, J = 4.8, 13.4 Hz, 1H), 4.40-4.29 (m, 3H), 4.28-4.18 (m, 1H), 2.97-2.85 (m, 1H), 2.60 (br d, J = 16.0 Hz, 1H), 2.41-2.31 (m, IH), 2.05-1.97 (m, 1H). MS (ESI) m/z 569.0 [M + H]⁺ |
| | 1-(3-chloro-4-iodophenyl)-3-((2-(2,6-dioxopiperidin-3-yl)-4-hydroxy-1- | |
| | oxoisoindolin-5-yl)methyl)urea | |
| IG-42 | 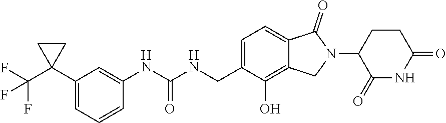 [SMILES: O=C1CCC(N2Cc3c(ccc(CNC(=O)Nc4cccc(C5(C(F)(F)F)CC5)c4)c3O)C2=O)C(=O)N1] | ¹H NMR (400 MHz, DMSO-d₆) δ = 10.97 (s, 1H), 10.06 (s, 1H), 8.84 (s, 1H), 7.56 (s, IH), 7.34 (t, J = 7.2 Hz, 2H), 7.26-7.18 (m, 2H), 7.00 (br d, J = 7.6 Hz, 1H), 6.74 (br t, J = 6.0 Hz, IH), 5.09 (dd, J = 5.2, 13.2 Hz, 1H), 4.39-4.32 (m, 3H), 4.27-4.19 (m, 1H), 2.96-2.84 (m, 1H), 2.60 (br d, J = 16.4 Hz, 1H), 2.42-2.29 (m, 1H), 2.06-1.95 (m, 1H), 1.34-1.26 (m, 2H), 1.07 (br s, 2H). |
| | 1-((2-(2,6-dioxopiperidin-3-yl)-4-hydroxy-1-oxoisoindolin-5-yl)methyl)-3-(3- | MS (ESI) m/z 517.3 [M + H]⁺ |
| | (1-(trifluoromethyl)cyclopropyl)phenyl)urea | |
| IG-43 | 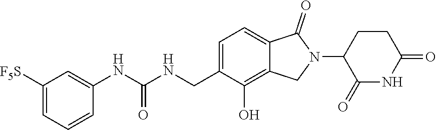 | ¹H NMR (400 MHz, DMSO-d₆) δ = 10.98 (br s, 1H), 10.07-9.89 (m, 1H), 9.31-9.20 (m, 1H), 8.27 (s, 1H), 7.52-7.43 (m, 2H), 7.43-7.38 (m, 1H), 7.35 (br d, J = 7.6 Hz, 1H), 7.20 (br d, J = 7.6 Hz, 1H), 6.94- 6.88 (m, 1H), 5.09 (br dd, J = 5.2, 13.2 Hz, 1H), 4.41- 4.31 (m, 3H), 4.29-4.20 (m, 1H), 2.97-2.86 (m, 1H), 2.67 (br s, 1H), 2.39-2.31 (m, 1H), 2.07-1.96 (m, 1H). |
| | 1-((2-(2,6-dioxopiperidin-3-yl)-4-hydroxy-1-oxoisoindolin-5-yl)methyl)-3-(3- | MS (ESI) m/7. 535.2 [M + H]⁺ |
| | (pentafluoro-l6-sulfaneyl)phenyl)urea | |
| IG-47 | 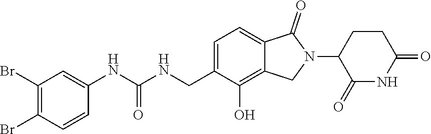 [SMILES: O=C1CCC(N2Cc3c(ccc(CNC(=O)Nc4ccc(Br)c(Br)c4)c3O)C2=O)C(=O)N1] | ¹H NMR (400 MHz, DMSO-d₆) δ = 10.98 (s, 1H), 9.96 (br s, 1H), 9.03 (s, 1H), 7.97 (d, J = 2.4 Hz, 1H), 7.57 (d, J = 8.8 Hz, 1H), 7.34 (d, J = 7.6 Hz, 1H), 7.26-7.16 (m, 2H), 6.88-6.80 (m, 1H), 5.13-5.06 (m, 1H), 4.40-4.33 (m, 3H), 4.27-4.20 (m, 1H), 2.96-2.85 (m, 1H), 2.62 (br s, 1H), 2.40-2.34 (m, 1H), 2.05-1.97 (m, 1H). MS (ESI) m/z 565.0, 567.0, 569.0 [M + H]⁺ |
| | 1-(3,4-dibromophenyl)-3-((2-(2,6-dioxopiperidin-3-yl)-4-hydroxy-1- | |
| | oxoisoindolin-5-yl)methyl)urea | |
| IG-48 | 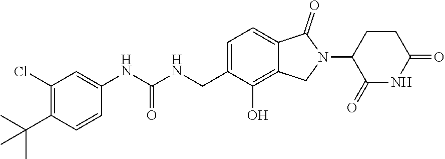 [SMILES: CC(C)(C)c1ccc(NC(=O)NCc2ccc3c(c2O)CN(C2CCC(=O)NC2=O)C3=O)cc1Cl] | ¹H NMR (400 MHz, DMSO-d₆) δ = 11.00 (s, 1H), 10.08 (br s, 1H), 8.89 (s, 1H), 7.60 (d, J = 2.4 Hz, 1H), 7.34 (d, J = 7.6 Hz, 1H), 7.31 (d, J = 8.8 Hz, 1H), 7.20 (d, J = 7.6 Hz, 1H), 7.16 (dd, J = 2.4, 8.8 Hz, 1H), 6.79 (br t, J = 5.2 Hz, 1H), 5.10 (dd, J = 5.2, 13.2 Hz, 1H), 4.40-4.30 (m, 3H), 4.26-4.20 (m, 1H), 2.96-2.86 (m, 1H), 2.60 (br dd, J = 2.4, 15.6 Hz, 1H), 2.37 (br dd, J = 4.4, 13.2 Hz, 1H), 2.04- 1.98 (m, 1H), 1.40 (s, 9H). MS (ESI) m/z 499.2 [M + H]⁺ |
| | 1-(4-(tert-butyl)-3-chlorophenyl)-3-((2-(2,6-dioxopiperidin-3-yl)-4-hydroxy- | |
| | 1-oxoisoindolin-5-yl)methyl)urea | |
| IG-50 | 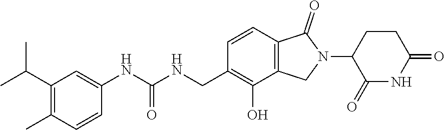 [SMILES: Cc1ccc(NC(=O)NCc2ccc3c(c2O)CN(C2CCC(=O)NC2=O)C3=O)cc1C(C)C] | ¹H NMR (400 MHz, DMSO-d₆) δ = 10.98 (s, 1H), 10.21 (s, 1H), 8.62 (s, 1H), 7.35 (d, J = 8.0 Hz, 1H), 7.20 (dd, J = 2.8, 4.9 Hz, 2H), 7.12 (dd, J = 2.6, 8.0 Hz, 1H), 6.97 (d, J = 8.0 Hz, 1H), 6.70-6.66 (m, 1H), 5.09 (dd, J = 5.2, 13.2 Hz, 1H), 4.38-4.31 (m, 3H), 4.25-4.19 (m, 1H), 3.03 (td, J = 7.2, 13.6 Hz, 1H), 2.96-2.85 (m, 1H), 2.59 (br d, J = 17.2 Hz, 1H), 2.37 (br s, IH), 2.20 (s, 3H), 2.04-1.97 (m, 1H), 1.14 |
| | 1-((2-(2,6-dioxopiperidin-3-yl)-4-hydroxy-1-oxoisoindolin-5-yl)methyl)-3-(3- | (d, J = 6.8 Hz, 6H). |
| | isopropyl-4-methylphenyl)urea | MS (ESI) m/z 465.1 [M + H]⁺ |
| IG-51 | 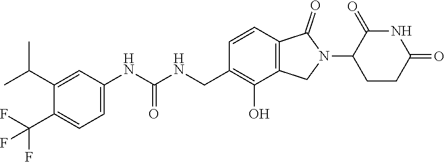 [SMILES: CC(C)c1cc(NC(=O)NCc2ccc3c(c2O)CN(C2CCC(=O)NC2=O)C3=O)ccc1C(F)(F)F] | |
| | 1-((2-(2,6-dioxopiperidin-3-yl)-4-hydroxy-1-oxoisoindolin-5-yl)methyl)-3-(3- | |
| | isopropyl-4-(trifluoromethyl)phenyl)urea | |
| IG-52 | 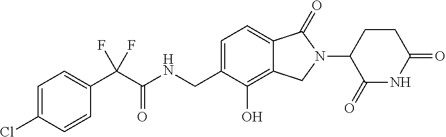 [SMILES: O=C1CCC(N2Cc3c(ccc(CNC(=O)C(F)(F)c4ccc(Cl)cc4)c3O)C2=O)C(=O)N1] | ¹H NMR (400 MHz, DMSO-d₆) δ = 10.99 (br s, 1H), 9.92-9.73 (m, 1H), 9.50 (br s, 1H), 7.63 (s, 4H), 7.20- 7.13 (m, 2H), 5.09 (dd, J = 5.2, 13.2 Hz, 1H), 4.41 (br d, J = 5.2 Hz, 2H), 4.38-4.32 (m, 1H), 4.28-4.20 (m, 1H), 2.96-2.86 (m, 1H), 2.64-2.57 (m, 1H), 2.35 (dq, J = 4.8, 13.2 Hz, 1H), 2.06-1.97 (m, 1H). MS (ESI) m/z 478.1 [M + H]⁺ |
| | 2-(4-chlorophenyl)-N-((2-(2,6-dioxopiperidin-3-yl)-4-hydroxy-1- | |
| | oxoisoindolin-5-yl)methyl)-2,2-difluoroacetamide | |
| IG-53 | 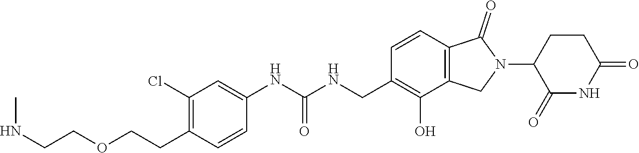 [SMILES: CNCCOCCc1ccc(NC(=O)NCc2ccc3c(c2O)CN(C2CCC(=O)NC2=O)C3=O)cc1Cl] | ¹H NMR (400 MHz, DMSO-d₆) δ = 10.98 (s, 1H), 10.07 (s, 1H), 9.19-9.14 (m, 1H), 8.68-8.53 (m, 2H), 7.63 (d, J = 2.0 Hz, 1H), 7.35 (d, J = 7.6 Hz, 1H), 7.26-7.23 (m, 1H), 7.21-7.16 (m, 2H), 6.98- 6.92 (m, 1H), 5.09 (dd, J = 5.2, 13.3 Hz, 1H), 4.39- 4.32 (m, 3H), 4.27-4.21 (m, 1H), 3.67-3.59 (m, 4H), 3.10-3.04 (m, 2H), 2.93-2.85 (m, 3H), 2.60 (br d, J = 17.6 Hz, 1H), 2.53 (t, J = 5.2 Hz, 3H), 2.41- 2.32 (m, 1H), 2.05-1.97 (m, 1H). LC-MS (ESI) m/z 544.2 [M + H]⁺ |
| | 1-(3-chloro-4-(2-(2-(methy lamino)ethoxy)ethyl)phenyl)-3-((2-(2,6- | |
| | dioxopiperidin-3-yl)-4-hydroxy-1-oxoisoindolin-5-yl)methyl)urea | |
| IG-55 | 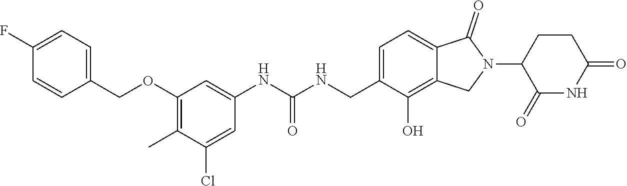 [SMILES: Cc1c(Cl)cc(NC(=O)NCc2ccc3c(c2O)CN(C2CCC(=O)NC2=O)C3=O)cc1OCc1ccc(F)cc1] | ¹H NMR (400 MHz, DMSO-d₆) δ −11.06-10.91 (m, 1H), 10.16-9.95 (m, 1H), 8.94-8.77 (m, 1H), 7.51 (dd, J = 5.6, 8.4 Hz, 2H), 7.34 (d, J = 7.6 Hz, 1H), 7.27-7.16 (m, 4H), 7.07 (s, 1H), 6.80 (br t, J = 5.6 Hz, 1H), 5.13-5.04 (m, 3H), 4.40-4.30 (m, 3H), 4.28-4.20 (m, 1H), 2.96-2.86 (m, 1H), 2.65-2.56 (m, 1H), 2.43-2.31 (m, 1H), 2.14 (s, 3H), 2.05-1.98 (m, 1H). LC-MS (ESI) m/z 581.3 [M + H]⁺ |
| | 1-(3-chloro-5-((4-fluorobenzyl)oxy)-4-methylphenyl)-3-((2-(2,6- | |
| | dioxopiperidin-3-yl)-4-hydroxy-1-oxoisoindolin-5-yl)methyl)urea | |
| IG-56 | 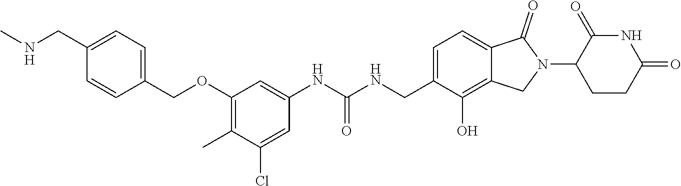 [SMILES: CNCc1ccc(COc2cc(NC(=O)NCc3ccc4c(c3O)CN(C3CCC(=O)NC3=O)C4=O)cc(Cl)c2C)cc1] | ¹H NMR (400 MHz, DMSO-d₆) δ −8.95-8.82 (m, 1H), 7.41-7.31 (m, 5H), 7.23-7.14 (m, 2H), 7.06 (s, 1H), 6.85- 6.76 (m, 1H), 5.09 (dd, J = 5.2, 13.1 Hz, 1H), 5.04 (s, 2H), 4.40-4.32 (m, 3H), 4.27-4.19 (m, 1H), 3.64 (s, 2H), 2.98- 2.85 (m, 1H), 2.68-2.57 (m, 1H), 2.39-2.33 (m, 1H), 2.26 (s, 3H), 2.14 (s, 3H), 2.04-1.98 (m, 1H). LC-MS (ESI) m/z 606.3 [M + H]⁺ |
| | 1-(3-chloro-4-methyl-5-((4-((methylamino)methyl)benzyl)oxy)phenyl)-3-((2- | |
| | (2,6-dioxopiperidin-3-yl)-4-hydroxy-1-oxoisoindolin-5-yl)methyl)urea | |

### Example 4 Synthesis of N-(3-chloro-4-(2-(2-(methylamino)ethoxy)ethyl)phenyl)-3-(2-(2,6-dioxopiperidin-3-yl)-1-oxoisoindolin-5-yl)propanamide (Compound IG-2)

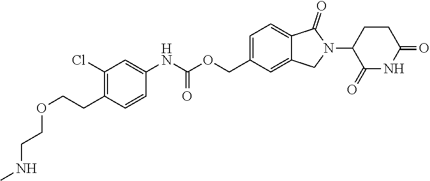 [SMILES: CNCCOCCc1ccc(NC(=O)OCc2ccc3c(c2)CN(C2CCC(=O)NC2=O)C3=O)cc1Cl]

### Step A. Preparation of Int 1r

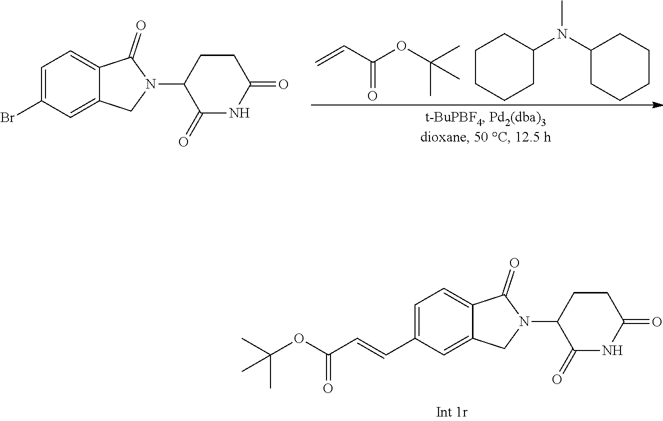

A mixture of N-cyclohexyl-N-methylcyclohexanamine (1.93 g, 9.90 mmol, 2.10 mL, 1.60 eq), tri-tert-butylphosphonium;tetrafluoroborate (900 mg, 3.10 mmol, 5.00 eq) and tris(dibenzylideneacetone)dipalladium(O) (566 mg, 618 umol, 0.100 eq) in dioxane (30.0 mL) was stirred at 25° C. for 0.5 h, then 3-(5-bromo-1-oxoisoindolin-2-yl)piperidine-2,6-dione (2.00 g, 6.19 mmol, 1.00 eq) and tert-butyl acrylate (1.98 g, 15.4 mmol, 2.25 mL, 2.50 eq) was added. The mixture was stirred at 50° C. for 12 h. The reaction was diluted with water (100 mL) and extracted with ethyl acetate (3×100 mL). The combined organic layers were washed with brine (100 mL), dried over anhydrous sodium sulfate, filtered and concentrated under reduced pressure to give a residue. The residue was purified by column chromatography (SiO₂, Petroleum ether/Ethyl acetate=1/1 to 0/1) to afford tert-butyl 3-(2-(2,6-dioxopiperidin-3-yl)-1-oxoisoindolin-5-yl)acrylate, Int 1r, (1.80 g, 4.86 mmol, 79% yield) as a brown solid. ¹H NMR (400 MHz, DMSO-d₆) δ=11.01 (br d, J=1.6 Hz, 1H), 7.97 (s, 1H), 7.84 (d, J=7.6 Hz, 1H), 7.74 (d, J=7.6 Hz, 1H), 7.67 (d, J=16.0 Hz, 1H), 6.67 (d, J=16.0 Hz, 1H), 5.14 (dd, J=5.2, 13.3 Hz, 1H), 4.55-4.44 (m, 1H), 4.41-4.28 (m, 1H), 2.92 (ddd, J=5.2, 13.6, 17.4 Hz, 1H), 2.62 (br s, 1H), 2.46-2.41 (m, 1H), 2.07-2.00 (m, 1H), 1.50 (s, 9H).

### Step B. Preparation of Int 1s

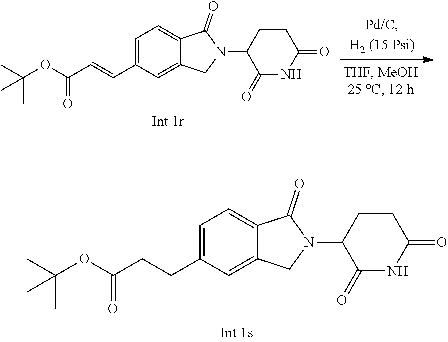 [SMILES: CC(C)(C)OC(=O)/C=C/c1ccc2c(c1)CN(C1CCC(=O)NC1=O)C2=O ; CC(C)(C)OC(=O)CCc1ccc2c(c1)CN(C1CCC(=O)NC1=O)C2=O]

To a solution of tert-butyl 3-(2-(2,6-dioxopiperidin-3-yl)-1-oxoisoindolin-5-yl)acrylate, Int 1r, (1.80 g, 4.86 mmol, 1.00 eq) in tetrahydrofuran (100 mL) and methanol (10.0 mL) was added palladium carbon (180 mg, 10% purity) at nitrogen atmosphere. The mixture was stirred under hydrogen atmosphere at 25° C. for 12 h. The reaction was filtered to give a filter cake. The filter cake was triturated with ethyl acetate (100 mL) for 10 min to afford tert-butyl 3-(2-(2,6-dioxopiperidin-3-yl)-1-oxoisoindolin-5-yl)propanoate, Int 1s, (1.20 g, 3.22 mmol, 66% yield) as a white solid. ¹H NMR (400 MHz, DMSO-d₆) δ=11.00 (br s, 1H), 7.64 (d, J=7.6 Hz, 1H), 7.46 (s, 1H), 7.38 (d, J=7.6 Hz, 1H), 5.11 (dd, J=4.8, 13.2 Hz, 1H), 4.47-4.36 (m, 1H), 4.35-4.21 (m, 1H), 2.93 (br t, J=7.2 Hz, 3H), 2.62 (br s, 1H), 2.59-2.56 (m, 2H), 2.40 (br dd, J=4.0, 13.3 Hz, 1H), 2.05-1.97 (m, 1H), 1.37 (d, J=1.2 Hz, 9H).

### Step C. Preparation of Int 1t

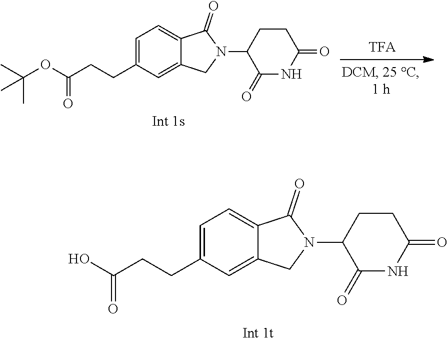 [SMILES: CC(C)(C)OC(=O)CCc1ccc2c(c1)CN(C1CCC(=O)NC1=O)C2=O ; O=C(O)CCc1ccc2c(c1)CN(C1CCC(=O)NC1=O)C2=O]

A mixture of tert-butyl 3-(2-(2,6-dioxopiperidin-3-yl)-1-oxoisoindolin-5-yl)propanoate, Int 1s, (1.20 g, 3.22 mmol, 1.00 eq) in dichloromethane (15.0 mL) and trifluoroacetic acid (3.00 mL) was stirred at 25° C. for 1 h. The reaction was concentrated under reduced pressure to afford 3-(2-(2,6-dioxopiperidin-3-yl)-1-oxoisoindolin-5-yl)propanoic acid, Int 1t, (1.00 g, 3.16 mmol, 98% yield) as a white solid. ¹H NMR (400 MHz, DMSO-d₆) δ=10.99 (s, 1H), 7.64 (d, J=7.6 Hz, 1H), 7.48 (s, 1H), 7.39 (d, J=7.6 Hz, 1H), 5.11 (dd, J=5.2, 13.2 Hz, 1H), 4.48-4.38 (m, 1H), 4.33-4.23 (m, 1H), 2.95 (br t, J=7.2 Hz, 3H), 2.63-2.58 (m, 3H), 2.40 (br dd, J=4.4, 13.2 Hz, 1H), 2.09-1.92 (m, 1H).

### Step D. Preparation of Int 1s

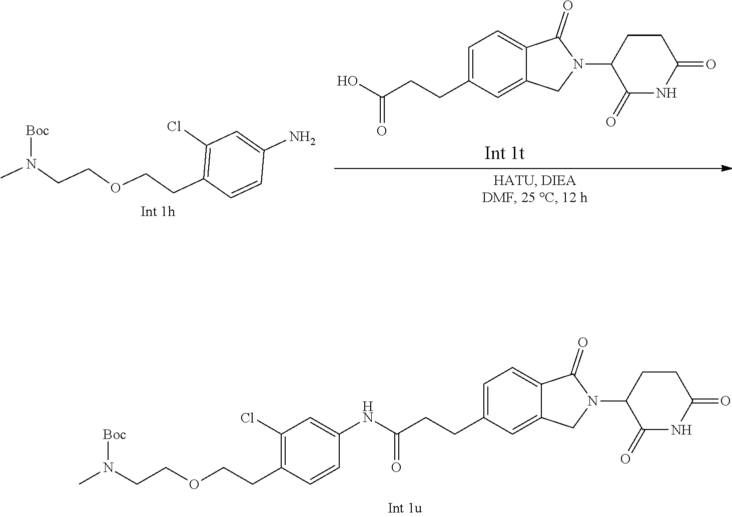 [SMILES: CN(CCOCCc1ccc(N)cc1Cl)C(=O)OC(C)(C)C ; O=C(O)CCc1ccc2c(c1)CN(C1CCC(=O)NC1=O)C2=O ; CN(CCOCCc1ccc(NC(=O)CCc2ccc3c(c2)CN(C2CCC(=O)NC2=O)C3=O)cc1Cl)C(=O)OC(C)(C)C]

To a solution of tert-butyl (2-(4-amino-2-chlorophenethoxy)ethyl)(methyl)carbamate, Int 1h, (1.14 g, 3.48 mmol, 1.10 eq) in dimethylformamide (10.0 mL) was added 3-(2-(2,6-dioxopiperidin-3-yl)-1-oxoisoindolin-5-yl)propanoic acid, Int 1t, (1.00 g, 3.16 mmol, 1.00 eq), 0-(7-azabenzotriazol-1-yl)-N,N,N,N-tetramethyluroniumhexafluorophosphate (1.20 g, 3.16 mmol, 1.00 eq) and N,N-diisopropylethylamine (1.23 g, 9.48 mmol, 1.65 mL, 3.00 eq), the mixture was stirred at 25° C. for 12 h. The reaction was diluted with water (50.0 mL) and extracted with ethyl acetate (3×50.0 mL). The combined organic layer was washed with brine (50.0 mL) and dried over anhydrous sodium sulfate, filtered and concentrated to give a residue. The residue was purified by column chromatography (SiO₂, Petroleum ether/Ethyl acetate=1/1 to 0/1) to afford tert-butyl (2-(2-chloro-4-(3-(2-(2,6-dioxopiperidin-3-yl)-1-oxoisoindolin-5-yl)propanamido)phenethoxy) ethyl)(methyl)carbamate, Int 1u, (1.50 g, 2.39 mmol, 76% yield) as a yellow solid. ¹H NMR (400 MHz, DMSO-d₆) δ=10.97 (s, 1H), 10.05 (s, 1H), 7.79 (s, 1H), 7.64 (d, J=8.0 Hz, 1H), 7.48 (s, 1H), 7.40 (d, J=8.0 Hz, 1H), 7.37-7.31 (m, 1H), 7.30-7.22 (m, 1H), 5.10 (dd, J=5.2, 13.4 Hz, 1H), 4.47-4.36 (m, 1H), 4.33-4.24 (m, 1H), 3.57 (br t, J=6.6 Hz, 2H), 3.47 (br t, J=5.6 Hz, 2H), 3.29-3.24 (m, 2H), 3.02 (br t, J=7.4 Hz, 2H), 2.88-2.83 (m, 2H), 2.70-2.69 (m, 1H), 2.68-2.65 (m, 2H), 2.61 (br s, 1H), 2.58-2.58 (m, 1H), 2.58-2.58 (m, 1H), 2.58-2.57 (m, 1H), 2.38 (br dd, J=4.4, 13.6 Hz, 1H), 1.37 (s, 9H).

### Step D. Preparation of Compound IG-2

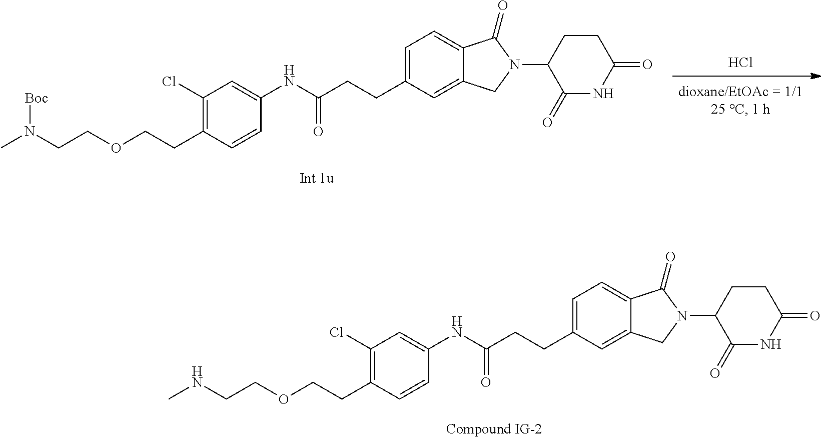 [SMILES: CN(CCOCCc1ccc(NC(=O)CCc2ccc3c(c2)CN(C2CCC(=O)NC2=O)C3=O)cc1Cl)C(=O)OC(C)(C)C ; Cl ; CNCCOCCc1ccc(NC(=O)CCc2ccc3c(c2)CN(C2CCC(=O)NC2=O)C3=O)cc1Cl]

To a solution of tert-butyl (2-(2-chloro-4-(3-(2-(2,6-dioxopiperidin-3-yl)-1-oxoisoindolin-5-yl)propanamido)phenethoxy)ethyl)(methyl)carbamate, Int 1u, (700 mg, 1.12 mmol, 1.00 eq) in ethyl acetate (100 mL) was added hydrochloric acid/dioxane (4 M, 10.0 mL, 35.8 eq), the mixture was stirred at 25° C. for 1 h. Two parallel reactions was combined. The mixture was concentrated to give a residue. The residue was trituration with methanol (2.00 mL) and filtered to give a residue, the residue was diluted with water (20.0 mL) and lyophilized to afford N-(3-chloro-4-(2-(2-(methylamino)ethoxy)ethyl)phenyl)-3-(2-(2,6-dioxopiperidin-3-yl)-1-oxoisoindolin-5-yl)propenamide, Compound IG-2, (813.28 mg, 1.40 mmol, 63% yield, 97% purity, hydrochloride) as a white solid. ¹H NMR (400 MHz, DMSO-d₆) δ=10.98 (br s, 1H), 10.21 (s, 1H), 8.60 (br s, 1H), 7.80 (d, J=1.6 Hz, 1H), 7.64 (d, J=8.0 Hz, 1H), 7.49 (s, 1H), 7.43-7.35 (m, 2H), 7.34-7.26 (m, 1H), 5.09 (dd, J=5.2, 13.6 Hz, 1H), 4.48-4.36 (m, 1H), 4.33-4.23 (m, 1H), 3.70-3.58 (m, 4H), 3.10-2.98 (m, 4H), 2.97-2.84 (m, 3H), 2.69 (br t, J=7.6 Hz, 2H), 2.63-2.56 (m, 1H), 2.52 (br s, 3H), 2.41-2.32 (m, 1H), 2.05-1.91 (m, 1H). MS (ESI) m/z 527.3 [M+H]⁺

The compounds in Table 6 were prepared in a manner similar to that described for Compound IG-2 using Int 1t and the appropriate aniline (c.f. Example 2).

TABLE 6

| IG No. | Structure | ¹H NMR/MS |
|---|---|---|
| IG-10 | 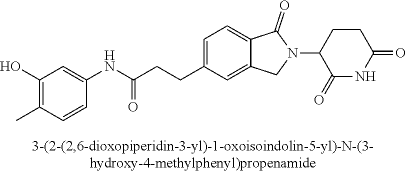 [SMILES: Cc1ccc(NC(=O)CCc2ccc3c(c2)CN(C2CCC(=O)NC2=O)C3=O)cc1O] | ¹H NMR (400 MHz, DMSO-d₆) δ = 10.97 (s, 1H), 9.70 (s, 1H), 9.24 (s, 1H), 7.64 (d, J = 7.6 Hz, 1H), 7.47 (s, 1H), 7.40 (d, J = 7.6 Hz, 1H), 7.21 (s, 1H), 6.91 (d, J = 8.0 Hz, 1H), 6.85-6.77 (m, 1H), 5.09 (dd, J = 5.1, 13.1 Hz, 1H), 4.44-4.25 (m, 2H), 3.01 (br t, J = 7.6 Hz, 2H), 2.92-2.87 (m, 1H), 2.62 (br d, J = 7.3 Hz, 3H), 2.39-2.33 (m, 1H), 2.03 (s, |
| | | 3H), 2.01-1.92 (m, 1H). MS |
| | | (ESI) m/z 422.2[M + H]⁺ |

### Example 5

### Synthesis of (2-(2,6-dioxopiperidin-3-yl)-1-oxoisoindolin-5-yl)methyl (3-chloro-4-(2-(2-(methylamino)ethoxy)ethyl)phenyl)carbamate (Compound IG-4)

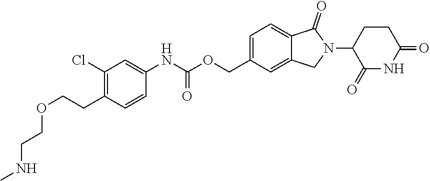 [SMILES: CNCCOCCc1ccc(NC(=O)OCc2ccc3c(c2)CN(C2CCC(=O)NC2=O)C3=O)cc1Cl]

### Step A. Preparation of Int 1v

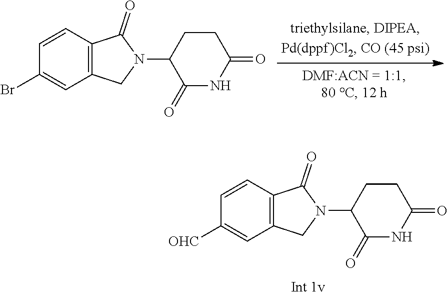 [SMILES: O=C1CCC(N2Cc3cc(Br)ccc3C2=O)C(=O)N1 ; O=Cc1ccc2c(c1)CN(C1CCC(=O)NC1=O)C2=O]

A mixture of 3-(5-bromo-1-oxo-isoindolin-2-yl)piperidine-2,6-dione (500 mg, 1.55 mmol, 1.00 eq), [1,1-bis(diphenylphosphino)ferrocene]dichloropalladium(II) (340 mg, 464 umol, 0.300 eq), N,N-diisopropylethylamine (1.00 g, 7.74 mmol, 1.35 mL, 5.00 eq), triethylsilane (1.80 g, 15.47 mmol, 2.47 mL, 10.0 eq) in dimethylformamide (5.00 mL) and acetonitrile (5.00 mL), the mixture was stirred at 80° C. for 12 h under carbonic oxide (45 psi) atmosphere. The mixture was filtered to give a filtrate. The filtrate was purified by reversed phase-HPLC (column: Shim-pack C18 150*25*10 um; mobile phase: [water(0.1% formic acid)-acetonitrile]) to afford 2-(2,6-dioxopiperidin-3-yl)-1-oxoisoindoline-5-carbaldehyde, Int 1v, (120 mg, 441 umol, 28% yield) as a yellow solid. ¹H NMR (400 MHz, DMSO-d₆) δ=11.04 (s, 1H), 10.16 (s, 1H), 8.16 (s, 1H), 8.07 (d, J=7.8 Hz, 1H), 7.95 (d, J=7.8 Hz, 1H), 5.17 (br dd, J=5.0, 13.4 Hz, 1H), 4.64-4.54 (m, 1H), 4.50-4.41 (m, 1H), 2.98-2.87 (m, 1H), 2.64 (br s, 1H), 2.46-2.35 (m, 1H), 2.05 (br dd, J=4.8, 10.8 Hz, 1H).

### Step B. Preparation of Int CC

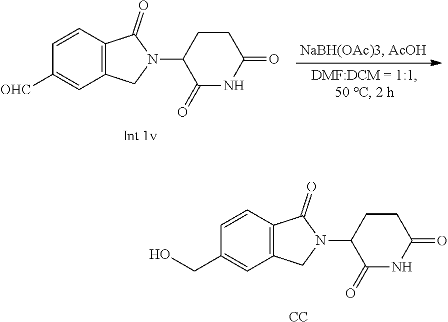 [SMILES: O=Cc1ccc2c(c1)CN(C1CCC(=O)NC1=O)C2=O ; O=C1CCC(N2Cc3cc(CO)ccc3C2=O)C(=O)N1 ; "CC"]

To a solution of 2-(2,6-dioxopiperidin-3-yl)-1-oxoisoindoline-5-carbaldehyde, Int 1v, (115 mg, 422 umol, 1.00 eq) in dimethylformamide (1.00 mL) and dichloromethane (1.00 mL) was added sodium borohydride acetate (448 mg, 2.11 mmol, 5.00 eq) and acetic acid (127 mg, 2.11 mmol, 120 uL, 5.00 eq), the mixture was stirred at 50° C. for 2 h. The reaction mixture was concentrated under reduced pressure to give a residue. The residue was purified by Prep-HPLC (column: Waters Atlantis T3 150*30 mm*5 um; mobile phase: [water(formic acid)-acetonitrile]; B %: 5%-35%, 10 min) to afford 3-(5-(hydroxymethyl)-1-oxoisoindolin-2-yl)piperidine-2,6-dione, Int CC, (60.0 mg, 219 umol, 52% yield) as a white solid. ¹H NMR (400 MHz, DMSO-d₆) δ=11.00 (s, 1H), 7.68 (d, J=7.8 Hz, 1H), 7.55 (s, 1H), 7.45 (d, J=7.8 Hz, 1H), 5.41 (t, J=5.6 Hz, 1H), 5.12 (dd, J=5.2, 13.2 Hz, 1H), 4.65-4.60 (m, 2H), 4.49-4.42 (m, 1H), 4.35-4.28 (m, 1H), 2.99-2.87 (m, 1H), 2.60 (br d, J=17.8 Hz, 1H), 2.40 (br dd, J=4.2, 13.2 Hz, 1H), 2.06-1.96 (m, 1H)

### Step C. Preparation of Int 1w

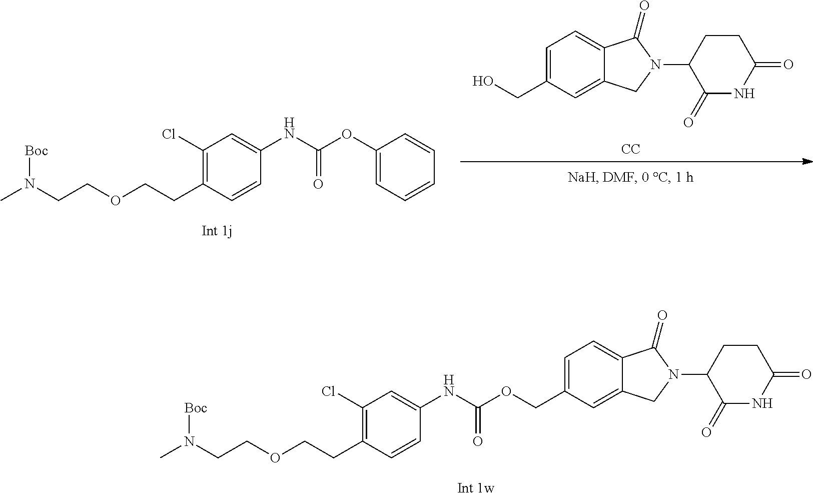 [SMILES: O=C1CCC(N2Cc3cc(CO)ccc3C2=O)C(=O)N1 ; CN(CCOCCc1ccc(NC(=O)Oc2ccccc2)cc1Cl)C(=O)OC(C)(C)C ; "CC" ; CN(CCOCCc1ccc(NC(=O)OCc2ccc3c(c2)CN(C2CCC(=O)NC2=O)C3=O)cc1Cl)C(=O)OC(C)(C)C]

To a solution of 3-(5-(hydroxymethyl)-1-oxoisoindolin-2-yl)piperidine-2,6-dione, Int 1j, (397 mg, 1.45 mmol, 1.00 eq) in N,N-dimethylformamide (8.00 mL) was added sodium hydride (116 mg, 2.90 mmol, 60% purity, 2.00 eq) were stirred at 0° C. for 5 min, followed by addition of tert-butyl (2-(2-chloro-4-((phenoxycarbonyl)amino)phenethoxy)ethyl)(methyl)carbamate, Int CC, (650 mg, 1.45 mmol, 1.00 eq). The mixture was stirred at 0° C. for 1 h under nitrogen atmosphere. The mixture was quenched with formic acid (10.0 mL), diluted with water (20.0 mL) and extracted with ethyl acetate (3×20.0 mL). The combined organic layers were washed with brine (2×10.0 mL), dried over sodium sulfate and filtered. The filtrate was concentrated under reduced pressure to give a residue. The residue was purified by reversed-phase HPLC (C18, 12 g; condition: water/acetonitrile=1/0-0/1, 0.1% formic acid) and lyophilized to afford tert-butyl (2-(2-chloro-4-((((2-(2,6-dioxopiperidin-3-yl)-1-oxoisoindolin-5-yl)methoxy)carbonyl)amino)phenethoxy)ethyl)(methyl)carbamate, Int 1w, (410 mg, 450 umol, 31% yield, 69% purity) as a yellow solid. MS (ESI) m/z 529.0 [M-99]⁺

### Step D. Preparation of Compound IG-4

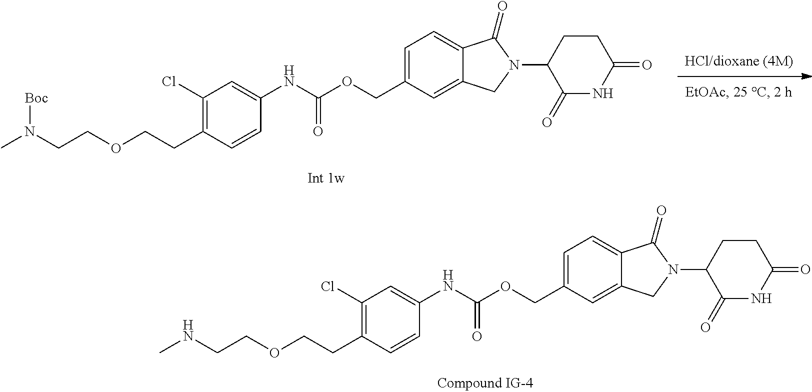 [SMILES: CN(CCOCCc1ccc(NC(=O)OCc2ccc3c(c2)CN(C2CCC(=O)NC2=O)C3=O)cc1Cl)C(=O)OC(C)(C)C ; "HCl/dioxane (4M)" ; CNCCOCCc1ccc(NC(=O)OCc2ccc3c(c2)CN(C2CCC(=O)NC2=O)C3=O)cc1Cl]

To a solution of tert-butyl (2-(2-chloro-4-((((2-(2,6-dioxopiperidin-3-yl)-1-oxoisoindolin-5-yl)methoxy)carbonyl)amino)phenethoxy)ethyl)(methyl)carbamate, Int 1w, (500 mg, 795 umol, 1.00 eq) in ethyl acetate (5.00 mL) was added hydrochloric acid/dioxane (4.00 M, 5.00 mL, 25.2 eq). The mixture was stirred at 25° C. for 2 h. The mixture was concentrated under reduced pressure to give a residue. The residue was triturated with ethyl acetate (10.0 mL) and filtered to give a residue, the residue was purified by Prep-HPLC (column: 3_Phenomenex Luna C18 75*30 mm*3 um; mobile phase: [water(HCl)-ACN]; B %: 12%-32%, 8 min) and lyophilized to afford (2-(2,6-dioxopiperidin-3-yl)-1-oxoisoindolin-5-yl)methyl (3-chloro-4-(2-(2-(methylamino)ethoxy)ethyl)phenyl)carbamate, Compound IG-4, (242 mg, 452 umol, 57% yield, 99% purity) as a yellow solid. ¹H NMR (400 MHz, DMSO+D₂O) δ=7.74 (d, J=7.6 Hz, 1H), 7.61 (s, 1H), 7.57-7.51 (m, 2H), 7.27 (s, 2H), 5.23 (s, 2H), 5.02 (dd, J=5.2, 13.3 Hz, 1H), 4.53-4.41 (m, 1H), 4.38-4.29 (m, 1H), 3.66-3.55 (m, 4H), 3.03 (t, J=5.0 Hz, 2H), 2.94-2.77 (m, 3H), 2.62 (br dd, J=2.4, 15.6 Hz, 1H), 2.52 (br s, 3H), 2.44-2.29 (m, 1H), 2.08-1.96 (m, 1H). MS (ESI) m/z 529.1 [M+H]⁺

The compounds in Table 7 were prepared in a manner similar to that described for Compound IG-4 using Int CC and the appropriately substituted aryl isocyanate, or phenyl carbamate (c.f. Example 2).

TABLE 7

| IG No. | Structure | ¹H NMR/MS |
|---|---|---|
| IG-13 | 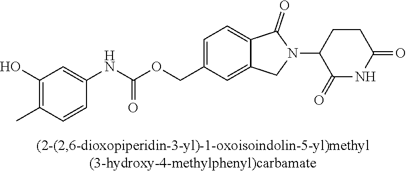 [SMILES: Cc1ccc(NC(=O)OCc2ccc3c(c2)CN(C2CCC(=O)NC2=O)C3=O)cc1O] | ¹H NMR (400 MHz, DMSO-d₆) δ = 10.98 (s, 1H), 9.59 (br s, 1H), 9.24 (s, 1H), 7.75 (d, J = 7.6 Hz, 1H), 7.64 (s, 1H), 7.55 (d, J = 7.6 Hz, 1H), 7.06 (s, 1H), 6.91 (d, J = 8.0 Hz, 1H), 6.75 (dd, J = 1.8, 8.0 Hz, 1H), 5.25 (s, 2H), 5.12 (dd, J = 5.1, 13.2 Hz, 1H), 4.53-4.29 (m, 2H), 2.91 (ddd, J = 5.3, 13.5, 17.6 Hz, 1H), 2.64- 2.55 (m, 1H), 2.45-2.35 (m, 1H), |
| | | 2.03 (s, 3H), 2.02-1.96 (m, 1H). |
| | | MS (ESI) m/z 424.2 [M + H]⁺ |

### Example 6

### Synthesis of 1-(3-chloro-4-(2-(2-(methylamino)ethoxy)ethyl)phenyl)-3-((2-(1-methyl-2,6-dioxopiperidin-3-yl)-1-oxoisoindolin-5-yl)methyl)urea (Compound IG-8)

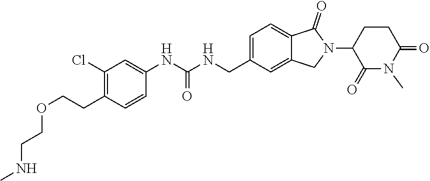 [SMILES: CNCCOCCc1ccc(NC(=O)NCc2ccc3c(c2)CN(C2CCC(=O)N(C)C2=O)C3=O)cc1Cl]

### Step A. Preparation of Int 1x

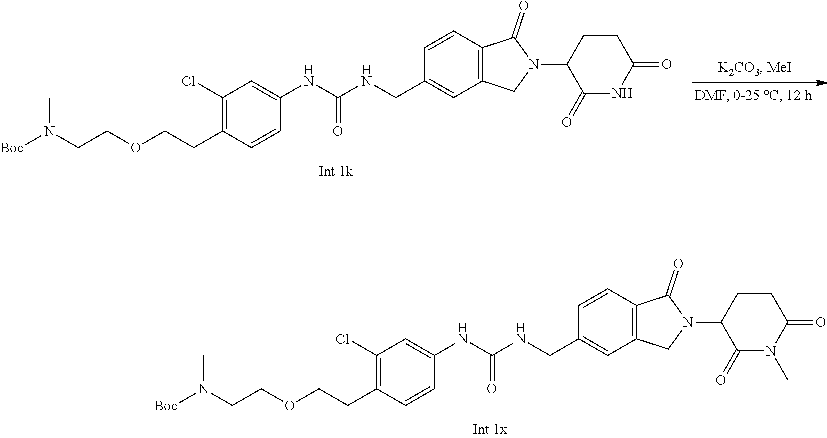 [SMILES: CN(CCOCCc1ccc(NC(=O)NCc2ccc3c(c2)CN(C2CCC(=O)NC2=O)C3=O)cc1Cl)C(=O)OC(C)(C)C ; CN(CCOCCc1ccc(NC(=O)NCc2ccc3c(c2)CN(C2CCC(=O)N(C)C2=O)C3=O)cc1Cl)C(=O)OC(C)(C)C]

To a solution of tert-butyl (2-(2-chloro-4-(3-((2-(2,6-dioxopiperidin-3-yl)-1-oxoisoindolin-5-yl)methyl)ureido)phenethoxy)ethyl)(methyl)carbamate, Int 1k, (100 mg, 159 umol, 1.00 eq) in N,N-dimethylformamide (1.00 mL) was added potassium carbonate (44.0 mg, 318 umol, 2.00 eq) and methyl iodide (22.6 mg, 159 umol, 9.91 uL, 1.00 eq) at 0° C. The mixture was stirred at 25° C. for 12 h. The mixture was quenched with saturated ammonium chloride solution (5.00 mL) and concentrated to give a residue. The residue was purified by Prep-HPLC (column: Phenomenex Luna C18 150*25 mm*10 um; mobile phase: [water(FA)-ACN]; B %: 43%-73%, 10 min) and lyophilized to afford tert-butyl (2-(2-chloro-4-(3-((2-(1-methyl-2,6-dioxopiperidin-3-yl)-1-oxoisoindolin-5-yl)methyl)ureido)phenethoxy)ethyl)(methyl)carbamate, Int 1x, (60.0 mg, 93.4 umol, 58% yield) as a yellow solid. ¹H NMR (400 MHz, DMSO-d₆) δ=8.83 (s, 1H), 7.73-7.65 (m, 2H), 7.51 (s, 1H), 7.44 (d, J=8.0 Hz, 1H), 7.23-7.12 (m, 2H), 6.86 (t, J=6.0 Hz, 1H), 5.17 (dd, J=4.8, 13.4 Hz, 1H), 4.50-4.25 (m, 4H), 3.55 (br t, J=6.8 Hz, 2H), 3.47 (t, J=5.6 Hz, 2H), 3.30-3.25 (m, 4H), 2.99-2.93 (m, 1H), 2.76 (br d, J=5.2 Hz, 5H), 2.52 (br s, 1H), 2.47-2.39 (m, 1H), 2.38-2.31 (m, 1H), 2.06-1.96 (m, 1H), 1.37 (s, 9H).

MS (ESI) m/z 542.2 [M-100+H]⁺

### Step B. Preparation of Compound IG-8

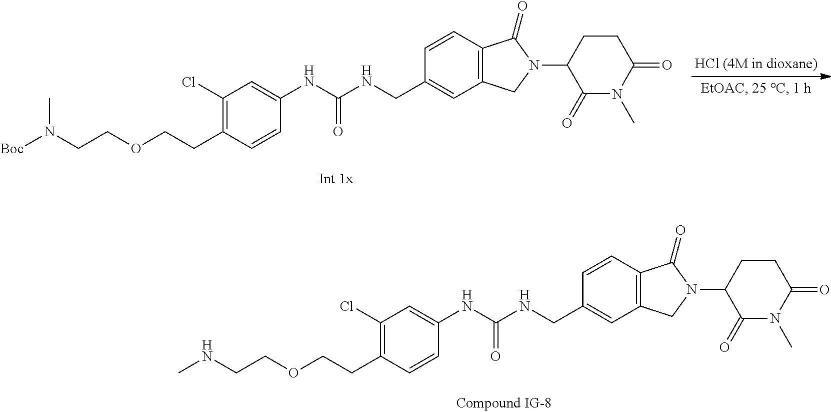 [SMILES: CN(CCOCCc1ccc(NC(=O)NCc2ccc3c(c2)CN(C2CCC(=O)N(C)C2=O)C3=O)cc1Cl)C(=O)OC(C)(C)C ; CNCCOCCc1ccc(NC(=O)NCc2ccc3c(c2)CN(C2CCC(=O)N(C)C2=O)C3=O)cc1Cl]

To a solution of tert-butyl (2-(2-chloro-4-(3-((2-(1-methyl-2,6-dioxopiperidin-3-yl)-1-oxoisoindolin-5-yl)methyl)ureido)phenethoxy)ethyl)(methyl)carbamate, Int 1x, (60.0 mg, 93.4 umol, 1.00 eq) in ethyl acetate (1.00 mL) was added hydrochloric acid/dioxane (4.00 M, 23.4 uL, 1.00 eq). The mixture was stirred at 25° C. for 1 h. The mixture was concentrated to afford 1-(3-chloro-4-(2-(2-(methylamino)ethoxy)ethyl)phenyl)-3-((2-(1-methyl-2,6-dioxopiperidin-3-yl)-1-oxoisoindolin-5-yl)methyl)urea, Compound IG-8, (34.1 mg, 62.9 umol, 67% yield) as a yellow solid. ¹H NMR (400 MHz, DMSO-d₆) δ=9.18-8.96 (m, 1H), 8.81-8.37 (m, 2H), 7.75-7.62 (m, 2H), 7.51 (s, 11H), 7.44 (d, J=7.6 Hz, 1H), 7.28-7.16 (m, 2H), 7.05-6.92 (m, 1H), 5.17 (dd, J=5.2, 13.5 Hz, 1H), 4.51-4.25 (m, 4H), 3.69-3.56 (m, 4H), 3.08 (br s, 2H), 3.04-2.93 (m, 4H), 2.89 (br t, J=6.8 Hz, 2H), 2.81-2.69 (m, 1H), 2.54 (s, 3H), 2.39 (br dd, J=4.0, 13.3 Hz, 1H), 207-1.94 (m, 1H). MS (ESI) m/z 542.2 [M+H]⁺

### Example 7

### Synthesis of N-(2-(2,6-dioxopiperidin-3-yl)-1,3-dioxoisoindolin-5-yl)-2-(trifluoromethoxy)benzenesulfonamide (Compound IG-9)

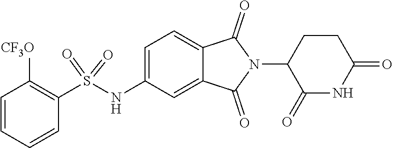

### Step A. Preparation of Compound IG-9

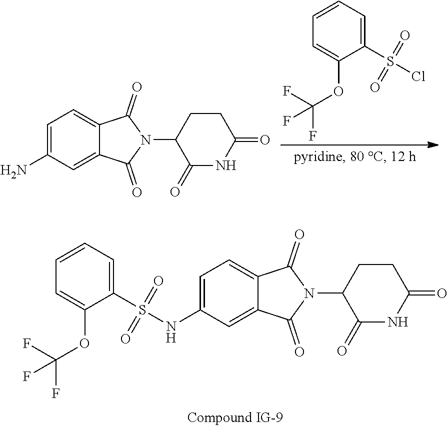

A mixture of 5-amino-2-(2,6-dioxopiperidin-3-yl)isoindoline-1,3-dione (500 mg, 183 mmol, 1.00 eq) and 2-(trifluoromethoxy)benzene-1-sulfonyl chloride (954 mg, 3.66 mmol, 2.00 eq) in pyridine (8 mL) was stirred at 80° C. for 12 h. The mixture was concentrated to give a residue. The residue was purified by Prep-HPLC (column: Phenomenex luna C18 150*25 mm*10 um; mobile phase: [water(FA)-ACN]; B %: 36%-66%, 10 min) and lyophilized to afford N-(2-(2,6-dioxopiperidin-3-yl)-1,3-dioxoisoindolin-5-yl)-2-(trifluoromethoxy)benzenesulfonamide, Compound IG-9, (409.01 mg, 814 umol, 44% yield, 99% purity) as an off-white solid. ¹H NMR (400 MHz, DMSO-d₆) δ=11.60 (s, 1H), 11.10 (s, 1H), 8.10 (dd, J=1.6, 8.0 Hz, 1H), 7.91-7.76 (m, 2H), 7.70-7.56 (m, 2H), 7.55-7.46 (m, 2H), 5.09 (dd, J=5.6, 12.8 Hz, 1H), 2.93-2.80 (m, IH), 2.60 (br d, J=2.4 Hz, 1H), 2.43 (br d, J=4.4 Hz, 1H), 2.07-1.96 (m, 1H) MS (ESI) m/z 498.2 [M+H]+

The compounds in Table 8 were prepared in a manner similar to that described for Compound IG-9 using 5-amino-2-(2,6-dioxopiperidin-3-yl)isoindoline-1,3-dione and the appropriately substituted aryl sulfonyl chloride (c.f. Example 2).

TABLE 8

| IG No. | Structure | ¹H NMR/MS |
|---|---|---|
| IG-11 | 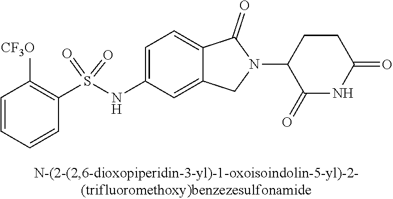 | ¹H NMR (400 MHz, DMSO-d₆) δ = 11.04 (s, 1H), 10.93 (s, 1H), 8.01 (dd, J = 1.6, 8.0 Hz, 1H), 7.75 (dt, J = 1.6, 7.9 Hz, 1H), 7.63-7.49 (m, 3H), 7.29 (s, 1H), 7.18 (dd, J = 1.6, 8.2 Hz, 1H), 5.02 (dd, J = 4.8, 13.4 Hz, 1H), 4.40-4.28 (m, 1H), 4.25- 4.12 (m, 1H), 3.27 (br s, 2H), 2.92- 2.80 (m, 1H), 2.57 (br d, J = 2.2 Hz, 1H), 2.31-2.22 (m, 1H), 1.97- 1.89 (m, 1H). MS (ESI) m/z 484.1 [M + H]⁺ |

### Example 8

### Synthesis of 1-(3-chloro-4-methylphenyl)-3-((2-(2,6-dioxopiperidin-3-yl)-3-oxoisoindolin-5-yl)methyl)urea (Compound IG-16)

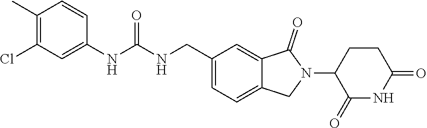 [SMILES: Cc1ccc(NC(=O)NCc2ccc3c(c2)C(=O)N(C2CCC(=O)NC2=O)C3)cc1Cl]

### Step A. Preparation of Int 1y

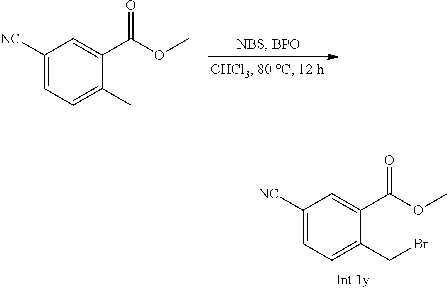

A solution of methyl 5-cyano-2-methylbenzoate (5.00 g, 28.5 mmol, 1.00 eq) and benzoyl peroxide (BPO) (691 mg, 2.85 mmol, 0.10 eq) in trichloromethane (50.0 mL) was added N-bromosuccinimide (6.10 g, 34.3 mmol, 1.20 eq), the mixture was stirred at 80° C. for 12 h. The reaction mixture was poured into water (50.0 m1) and extracted with ethyl acetate (3×50 mL), the combined organic phase was washed with brine (50.0 mL), dried with anhydrous sodium sulfate, filtered and concentrated in vacuum to afford methyl 2-(bromomethyl)-5-cyano-benzoate, Int 1y, (7.20 g, crude) as yellow oil.

### Step B. Preparation of Int 1z

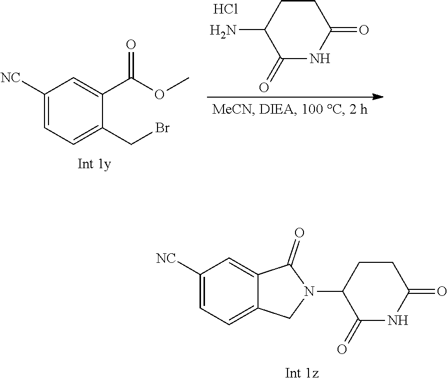 [SMILES: COC(=O)c1cc(C#N)ccc1CBr ; Cl ; NC1CCC(=O)NC1=O ; N#Cc1ccc2c(c1)C(=O)N(C1CCC(=O)NC1=O)C2]

To a solution of methyl 2-(bromomethyl)-5-cyanobenzoate, Int 1y, (7.20 g, 28.3 mmol, 1.00 eq) and 3-aminopiperidine-2,6-dione;hydrochloride (4.66 g, 28.3 mmol, 1.00 eq)) in acetonitrile (100 mL) was added N,N-diisopropylethylamine (11.0 g, 85.0 mmol, 14.8 mL, 3.00 eq), the mixture was stirred at 100° C. for 12 h. The mixture was filtered to give a residue. The residue was wash with citric acid (20 mL) to afford 2-(2,6-dioxopiperidin-3-yl)-3-oxoisoindoline-5-carbonitrile, Int 1z, (5.50 g, 20.4 mmol, 72% yield) as a purple solid. ¹H NMR (400 MHz, DMSO-d₆) δ=11.02 (s, 1H), 8.21 (s, 1H), 8.10 (dd, J=1.5, 7.9 Hz, 1H), 7.85 (d, J=7.9 Hz, 1H), 5.15 (dd, J=5.1, 13.3 Hz, 1H), 4.73-4.38 (m, 2H), 2.97-2.85 (m, 1H), 2.64-2.56 (m, 1H), 2.41 (br dd, J=4.5, 13.1 Hz, 1H), 2.09-1.97 (m, 1H). MS (ESI) m/z 270.2 [M+H]⁺

### Step C. Preparation of Int DD

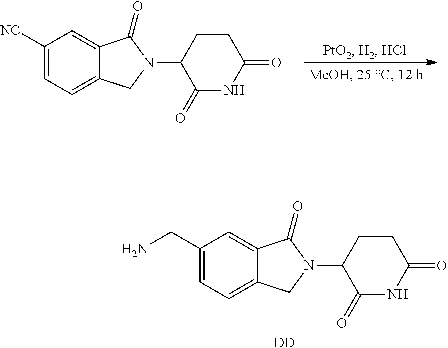

A solution of 2-(2,6-dioxopiperidin-3-yl)-3-oxoisoindoline-5-carbonitrile, Int 1z, (500 mg, 1.86 mmol, 1.00 eq) in methanol (100 mL) was added hydrochloric acid (28.3 g, 280 mmol, 10 mL, 36.0% purity, 54.3 eq) slowly and was degassed with nitrogen for 3 times. After that, platinum(IV) oxide (211 mg, 928 umol, 0.50 eq) was added under nitrogen atmosphere. The mixture was degassed with hydrogen for 3 times, and stirred at 25° C. for 12 h under hydrogen atmosphere (45 psi). The mixture was filtered to give a filter cake. The filter cake was dissolved by dimethyl formamide (10.0 mL). The solution was filtered to give a filtrate. The filtrate was concentrated in vacuum to afford 3-(6-(aminomethyl)-1-oxoisoindolin-2-yl)piperidine-2,6-dione, Int DD, (250 mg, crude) as a brown solid. ¹H NMR (400 MHz, DMSO-d₆) δ=11.01 (s, 1H), 7.90 (s, 1H), 7.75 (d, J=7.8 Hz, 1H), 7.70-7.57 (m, 1H), 5.12 (dd, J=5.1, 13.3 Hz, 1H), 4.55-4.44 (m, 1H), 4.40-4.29 (m, 1H), 4.15 (br s, 2H), 2.98-2.90 (m, 1H), 2.66-2.57 (m, 1H), 2.42 (dq, J=4.4, 13.2 Hz, 1H), 2.09-1.97 (m, 1H).

### Step D. Preparation of Compound IG-16

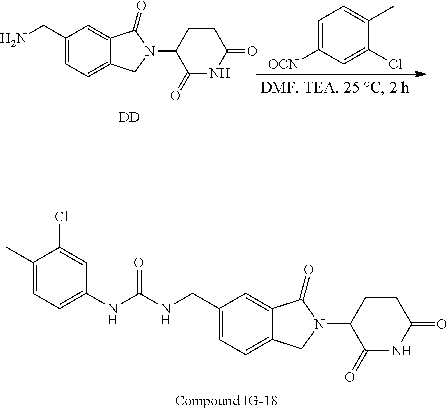

To a solution of 3-(6-(aminomethyl)-1-oxoisoindolin-2-yl)piperidine-2,6-dione, Int DD, (100 mg, 366 umol, 1.00 eq) in dimethyl formamide (3.00 mL) was added triethylamine (55.5 mg, 549 umol, 76.4 uL, 1.50 eq) and 2-chloro-4-isocyanato-1-methylbenzene (73.6 mg, 439 umol, 1.20 eq). The mixture was stirred at 25° C. for 2 h. The mixture was concentrated in vacuum to give a residue. The residue was purified by column chromatography (SiO2, Petroleum ether/Ethyl acetate=5/1 to 0/1) to afford the crude product. The crude product was purified by Prep-HPLC (column Phenomenex luna C18 150*25 mm*10 um; mobile phase: [water(FA)-ACN]; B %: 34%-54%, 9 min) and lyophilized to afford 1-(3-chloro-4-methylphenyl)-3-((2-(2,6-dioxopiperidin-3-yl)-3-oxoisoindolin-5-yl)methyl)urea, Compound IG-16, (31.4 mg, 71.2 umol, 10% yield, 99% purity) as a white solid. ¹H NMR (400 MHz, DMSO-d₆) δ=11.27-10.77 (m, 1H), 8.73 (s, 1H), 7.66 (s, 2H), 7.57 (s, 2H), 7.23-7.10 (m, 2H), 6.80 (t, J=6.2 Hz, 1H), 5.12 (dd, J=5.0, 13.0 Hz, 1H), 4.47-4.28 (m, 4H), 3.07-2.83 (m, 1H), 2.68-2.61 (m, 1H), 2.40-2.33 (m, 1H), 2.24 (s, 3H), 2.05-1.97 (m, 1H). MS (ESI) m/z 441.2 [M+H]⁺

The compounds in Table 9 were prepared in a manner similar to that described for Compound IG-16 using Int DD and the appropriately substituted aryl isocyanate, or phenyl carbamate (c.f. Example 2).

TABLE 9

| IG No. | Structure | ¹H NMR/MS |
|---|---|---|
| 20 | 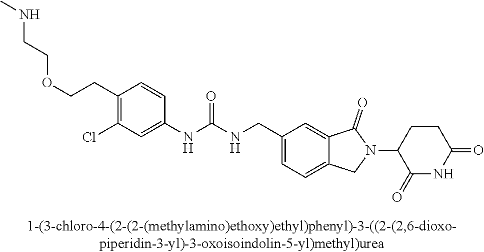 [SMILES: CNCCOCCc1ccc(NC(=O)NCc2ccc3c(c2)C(=O)N(C2CCC(=O)NC2=O)C3)cc1Cl] | ¹H NMR (400 MHz, DMSO-d₆) δ = 10.95 (s, 1H), 9.51 (br s, 1H), 8.94 (br s, 2H), 8.13 (s, 1H), 7.64-7.58 (m, 2H), 7.53 (s, 2H), 7.24-7.15 (m, 2H), 5.06 (dd, J = 5.2, 13.2 Hz, 1H), 4.43-4.24 (m, 4H), 3.65 (t, J = 5.2 Hz, 2H), 3.57 (br t, J = 7.2 Hz, 2H), 3.06-2.97 (m, 2H), 2.92-2.79 (m, 3H), 2.57 (br d, J = 18.0 Hz, 2H), 2.48-2.46 (m, 2H), 2.42-2.32 (m, 1H), 2.02-1.92 (m, 1H). MS (ESI) m/z 528.2 [M + H]⁺ |

### Example 8

### Synthesis of 4-amino-2-(2,6-dioxopiperidin-3-yl)-1,3-dioxoisoindolin-5-yl 2-(trifluoromethoxy)benzenesulfonate (Compound IG-17) and 6-amino-2-(2,6-dioxopiperidin-3-yl)-1,3-dioxoisoindolin-5-yl 2-(trifluoromethoxy)benzenesulfonate (Compound IG-18)

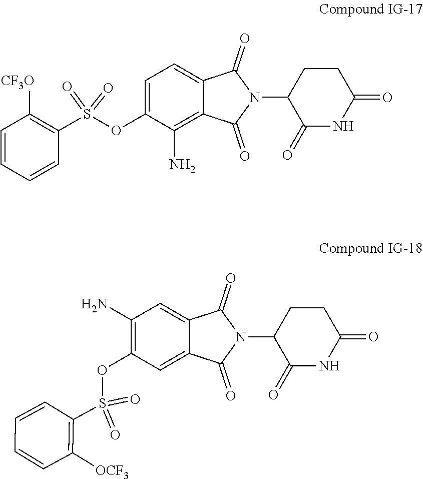

### Step A. Preparation of Int 2a and Int 2b

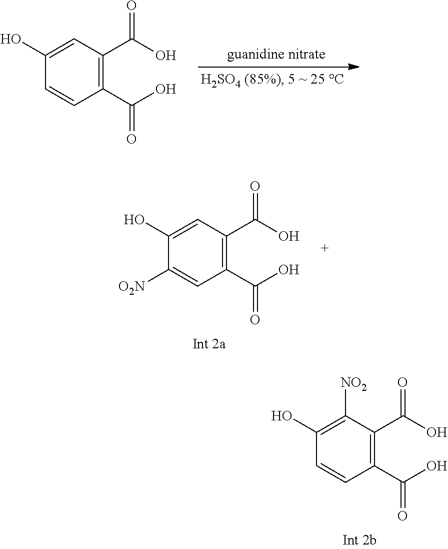 [SMILES: O=C(O)c1ccc(O)cc1C(=O)O ; O=C(O)c1cc(O)c([N+](=O)[O-])cc1C(=O)O ; O=C(O)c1ccc(O)c([N+](=O)[O-])c1C(=O)O]

4-hydroxyphthalic acid (10.0 g, 54.9 mmol, 1.00 eq) was suspension in sulfuric acid (200 mL, 85% purity) at 5° C. Guanidine nitrate (6.57 g, 53.8 mmol, 0.980 eq) was added to the mixture slowly at 5° C. Then the mixture was stirred at 25° C. for another 2 h. The reaction solution was poured into ice-water (500 mL) and extracted with ethyl acetate (500 mL). The organic layer was separated and concentrated under reduced pressure to afford a mixture of 4-hydroxy-5-nitrophthalic acid, Int 2a, and 4-hydroxy-3-nitrophthalic acid, Int 2b, (11.1 g, 29.3 mmol, 53% yield) as a yellow solid. ¹H NMR (400 MHz, DMSO-d₆) δ=13.18 (s, 1H), 12.09 (br s, 2H), 8.28 (s, 1H), 7.91 (d, J=8.8 Hz, 1H), 7.21-7.18 (m, 2H)

### Step B. Preparation of Int 2c and Int 2d

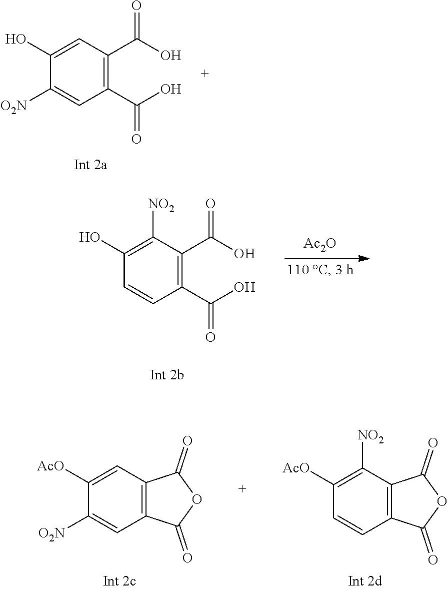 [SMILES: "Ac2O" ; O=C(O)c1cc(O)c([N+](=O)[O-])cc1C(=O)O ; O=C(O)c1ccc(O)c([N+](=O)[O-])c1C(=O)O ; "110 °C, 3 h" ; CC(=O)Oc1cc2c(cc1[N+](=O)[O-])C(=O)OC2=O ; CC(=O)Oc1ccc2c(c1[N+](=O)[O-])C(=O)OC2=O]

A mixture of 4-hydroxy-5-nitrophthalic acid, Int 2a, and 4-hydroxy-3-nitrophthalic acid, Int 2b, (11.1 g, 29.3 mmol, 60% purity, 1.00 eq) in acetic anhydride (50.0 mL) was degassed and purged with nitrogen for 3 times, and then the mixture was stirred at 110° C. for 3 h under nitrogen atmosphere. The reaction mixture was concentrated under reduced pressure to afford a mixture of 6-nitro-1,3-dioxo-1,3-dihydroisobenzofuran-5-yl acetate, Int 2c, and 4-nitro-1,3-dioxo-1,3-dihydroisobenzofuran-5-yl acetate, Int 2d, (6.00 g, 14.4 mmol, 49% yield) as black oil. ¹H NMR (400 MHz, DMSO-d₆) δ=8.79 (s, 1H), 8.42 (d, J=8.4 Hz, 1H), 8.35 (s, 1H), 8.15 (d, J=8.4 Hz, 1H), 2.38 (s, 3H), 2.22 (s, 3H).

### Step C. Preparation of Int 2e and Int 2f

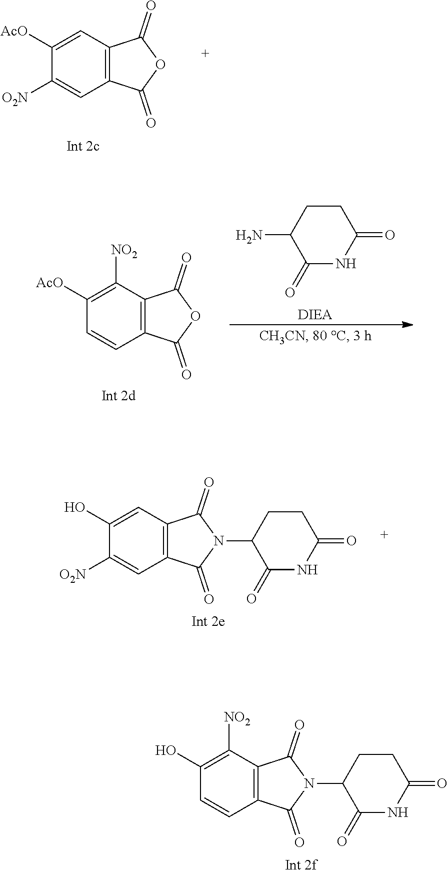

To a solution of a mixture of 6-nitro-1,3-dioxo-1,3-dihydroisobenzofuran-5-yl acetate, Int 2c, and 4-nitro-1,3-dioxo-1,3-dihydroisobenzofuran-5-yl acetate, Int 2d, (1.00 g, 2.39 mmol, 1.00 eq) in acetonitrile (20.0 mL) was added NN-diisopropylethylamine (618 mg, 4.78 mmol, 833 uL, 2.00 eq) and 3-aminopiperidine-2,6-dione (306 mg, 2.39 mmol, hydrochloric acid, 1.00 eq). The mixture was stirred at 80° C. for 3 h. The reaction mixture was concentrated under reduced pressure to give a residue. The residue was purified by reverse-phase HPLC (0.1% FA condition). The desired fraction was lyophilized to afford a mixture of 2-(2,6-dioxopiperidin-3-yl)-5-hydroxy-6-nitroisoindoline-1,3-dione, Int 2e, and 2-(2,6-dioxopiperidin-3-yl)-5-hydroxy-4-nitroisoindoline-1,3-dione, Int 2f, (400 mg, 1.25 mmol, 52% yield) as a white solid. ¹H NMR (400 MHz, DMSO-d₆) δ=11.14 (s, 1H), 8.36 (s, 1H), 7.96 (d, J=8.4 Hz, 1H), 7.50 (s, 1H), 7.48 (s, 1H), 7.48-7.46 (m, 11H), 5.16-5.12 (m, 1H), 2.95-2.81 (m, 2H), 2.62 (br s, 1H), 2.06 (br s, 1H).

### Step D. Preparation of Int 2g and Int 2h

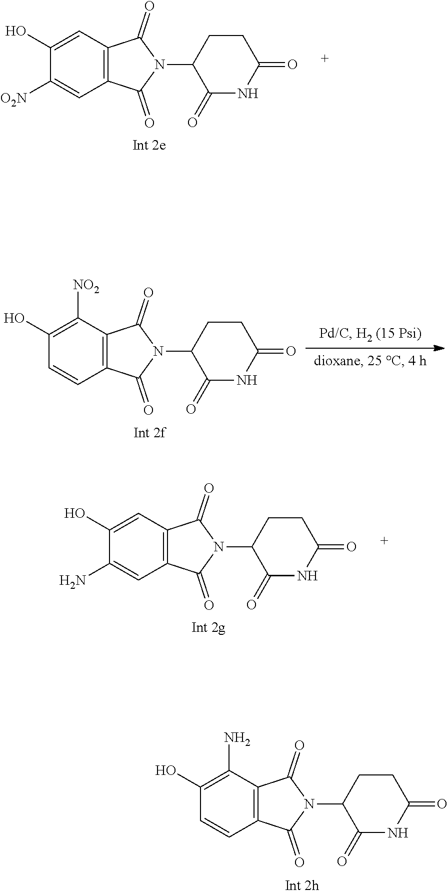 [SMILES: O=C1CCC(N2C(=O)c3cc(O)c([N+](=O)[O-])cc3C2=O)C(=O)N1 ; O=C1CCC(N2C(=O)c3ccc(O)c([N+](=O)[O-])c3C2=O)C(=O)N1 ; Nc1cc2c(cc1O)C(=O)N(C1CCC(=O)NC1=O)C2=O ; Nc1c(O)ccc2c1C(=O)N(C1CCC(=O)NC1=O)C2=O]

To a solution of a mixture of 2-(2,6-dioxopiperidin-3-yl)-5-hydroxy-6-nitroisoindoline-1,3-dione, Int 2e, and 2-(2,6-dioxopiperidin-3-yl)-5-hydroxy-4-nitroisoindoline-1,3-dione, Int 2f, (350 mg, 1.10 mmol, 1.00 eq) in dioxane (5.00 mL) was added palladium/carbon (100 mg, 10.0% purity) under nitrogen atmosphere. The suspension was degassed and purged with hydrogen for 3 times. The mixture was stirred under hydrogen atmosphere (15 Psi) at 25° C. for 3 h. The filter cake was diluted with water (10.0 mL). The residue was purified by reversed-phase HPLC(column: Phenomenex luna C18 150*25 mm*10 um; mobile phase: [water(FA)-ACN]; B %: 7%-37%, 10 min), and the fraction was lyophilized to afford a mixture of 5-amino-2-(2,6-dioxopiperidin-3-yl)-6-hydroxyisoindoline-1,3-dione, Int 2g, and 4-amino-2-(2,6-dioxopiperidin-3-yl)-5-hydroxyisoindoline-1,3-dione, Int 2h, (200 mg, 318.07 umol,) as a yellow solid. ¹H NMR (400 MHz, DMSO-d₆) δ=11.06 (s, 1H), 10.83 (br s, 1H), 6.97 (s, 1H), 6.93-6.90 (m, 1H), 5.95 (s, 2H), 4.99 (br d, J=5.4 Hz, 1H), 2.93-2.81 (m, 2H), 2.07-1.87 (m, 2H). MS (ESI) m/z 290.0 [M+H]⁺

### Step E. Preparation of Compound IG-17 and Compound IG-18

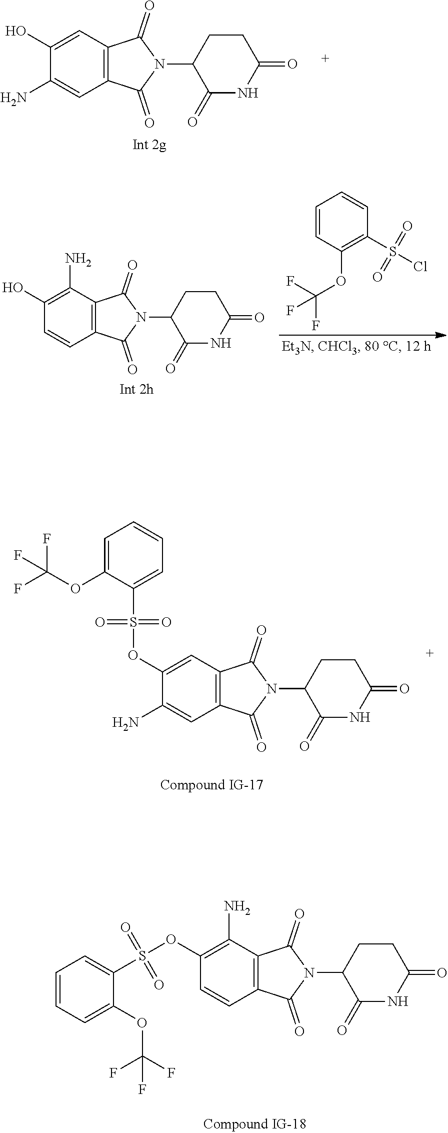

A mixture of 5-amino-2-(2,6-dioxopiperidin-3-yl)-6-hydroxyisoindoline-1,3-dione, Int 2g, and 4-amino-2-(2,6-dioxopiperidin-3-yl)-5-hydroxyisoindoline-1,3-dione, Int 2h, (60.0 mg, 104 umol, 1.00 eq), 2-(trifluoromethoxy)benzenesulfonyl chloride (54.0 mg, 207 umol, 2.00 eq) and triethylamine (31.5 mg, 311 umol, 43.3 uL, 3.00 eq) in trichloromethane (5.00 mL) was degassed and purged with nitrogen atmosphere for 3 times, and then the mixture was stirred at 80° C. for 12 h under nitrogen atmosphere. The mixture was filtered and the filtrate was concentrated to give a residue. The residue was purified by reverse-phase HPLC(column: Phenomenex luna C18 150*25 mm*10 um; mobile phase: [water(FA)-ACN]; B %: 38%-58%, 10 min), and the fraction was lyophilized to afford 6-amino-2-(2,6-dioxopiperidin-3-yl)-1,3-dioxoisoindolin-5-yl 2-(trifluoromethoxy)benzenesulfonate, Compound IG-17, (8.08 mg, 15.4 umol, 14.9% yield, 98% purity) as a yellow solid and 4-amino-2-(2,6-dioxopiperidin-3-yl)-1,3-dioxoisoindolin-5-yl 2-(trifluoromethoxy)benzenesulfonate, Compound IG-18, (20.85 mg, 39.4 umol, 38% yield, 97% purity) as a yellow solid.

Compound IG-17 ¹H NMR (400 MHz, DMSO-d₆) δ=11.08 (br s, 1H), 8.11 (dd, J=1.7, 7.9 Hz, 1H), 8.06-7.99 (m, 1H), 7.79 (br d, J=8.2 Hz, 1H), 7.69 (t, J=7.7 Hz, 1H), 7.18 (s, 1H), 7.11 (s, IH), 6.61 (br s, 2H), 5.03 (dd, J=5.3, 12.9 Hz, 1H), 2.93-2.79 (m, 1H), 2.59 (br d, J=2.8 Hz, 1H), 2.47-2.41 (m, 1H), 2.04-1.94 (m, 1H) MS (ESI) m/z 514.1 [M+H]⁺

Compound IG-18 ¹H NMR (400 MHz, DMSO-d₆) δ=11.12 (br s, 1H), 8.09 (d, J=7.6 Hz, 1H), 8.05-8.00 (m, 1H), 7.80 (br d, J=8.4 Hz, 1H), 7.71-7.66 (m, 1H), 7.11 (d, J=8.0 Hz, 1H), 7.01 (d, J=8.0 Hz, 1H), 6.41 (s, 2H), 5.07 (dd, J=5.6, 12.7 Hz, 1H), 2.90-2.82 (m, 1H), 2.61 (br s, 1H), 2.58-2.56 (m, 1H), 2.03 (br dd, J=4.8, 11.6 Hz, 1H) MS (ESI) m/z 514.1 [M+H]⁺

### Example 9

### Synthesis of 3-(4-((2-((2,3-dihydro-1H-inden-5-yl)methyl)pyrimidin-4-yl)amino)-1-oxoisoindolin-2-yl)piperidine-2,6-dione (Compound IG-19)

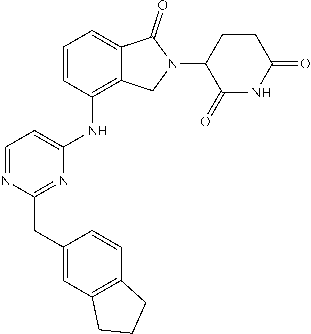 [SMILES: O=C1CCC(N2Cc3c(Nc4ccnc(Cc5ccc6c(c5)CCC6)n4)cccc3C2=O)C(=O)N1]

### Step A. Preparation of Int 2i

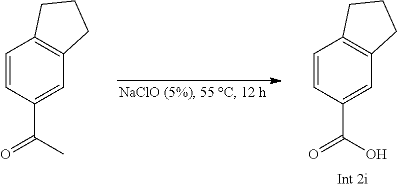

A solution of 1-(2,3-dihydro-1H-inden-5-yl)ethanone (24.0 g, 149 mmol, 1.00 eq) in sodium hypochlorite (500 mL, 5% purity) was stirred at 55° C. for 12 h. The reaction was adjusted to pH=9 with sodium hydroxide and extracted with ethyl acetate (3×50 mL). The organic layer was discarded. The water phase was adjusted to pH=4 with hydrochloric acid (36%) and extracted with ethyl acetate (3×50 mL). The organic layer was concentrated to give 2,3-dihydro-1H-indene-5-carboxylic acid, Int 2i, (20.0 g, 123 mmol, 82% yield) as a yellow solid. ¹H NMR (400 MHz, CDCl₃) δ=7.97 (s, 1H), 7.92 (d, J=7.8 Hz, 1H), 7.31 (d, J=7.8 Hz, 1H), 2.97 (br t, J=7.4 Hz, 4H), 2.17-2.10 (m, 2H). MS (ESI) m/z 163.2 [M+H]⁺

### Step B. Preparation of Int 2j

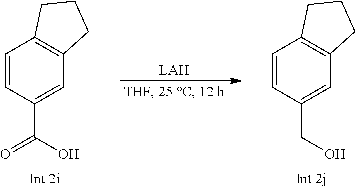 [SMILES: O=C(O)c1ccc2c(c1)CCC2 ; "LAH" ; "THF, 25 °C, 12 h" ; OCc1ccc2c(c1)CCC2]

To a solution of 2,3-dihydro-1H-indene-5-carboxylic acid, Int 2i, (10.0 g, 61.7 mmol, 1.00 eq) in tetrahydrofuran (100 mL) was added lithium aluminum hydride, LAH (7.02 g, 185 mmol, 3.00 eq) in four portions at 0° C. Then the mixture was stirred at 25° C. for 12 h. The reaction mixture was treated with water (7 mL), sodium hydroxide (15%, 7 mL) and water (7 mL) again. Then the mixture was dried over anhydrous sodium sulfate and filtered. The filtrate was concentrated to give (2,3-dihydro-1H-inden-5-yl)methanol, Int 2j, (8.80 g, crude) as a white solid. ¹H NMR (400 MHz, CDCl₃) δ=7.26 (s, 1H), 7.23 (d, J=7.8 Hz, 1H), 7.17-7.11 (m, 1H), 4.66 (d, J=5.2 Hz, 2H), 2.92 (dt, J=3.2, 7.2 Hz, 4H), 2.16-2.07 (m, 2H).

### Step C. Preparation of Int 2k

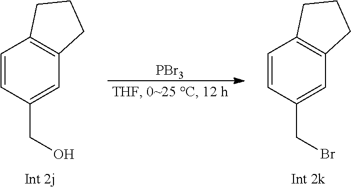

To a solution of (2,3-dihydro-1H-inden-5-yl)methanol, Int 2j, (7.80 g, 52.6 mmol, 1.00 eq) in dimethyl formamide (80.0 mL) was added phosphorus tribromide, PBr3 (7.12 g, 26.3 mmol, 0.500 eq) at 0° C. Then the mixture was stirred at 25° C. for 12 h. The reaction mixture was poured into sodium bicarbonate (Sat., 100 mL) at 0° C. and extracted with ethyl acetate (3×20 mL). The organic layer was dried over sodium sulfate and filtered. The filtrate was concentrated to give a residue. The residue was purified by flash silica gel chromatography (ISCO®; 120 g SepaFlash® Silica Flash Column, Eluent of 0~10% ethyl acetate/petroleum ether gradient @100 mL/min) to afford 5-(bromomethyl)-2,3-dihydro-1H-indene, Int 2k, (8.00 g, 37.9 mmol, 72% yield) as yellow oil. ¹H NMR (400 MHz, CDCl₃) δ=7.27 (s, 1H), 7.23-7.12 (m, 2H), 4.52 (s, 2H), 2.90 (dt, J=2.6, 7.4 Hz, 4H), 2.15-2.06 (m, 2H).

### Step D. Preparation of Int 21

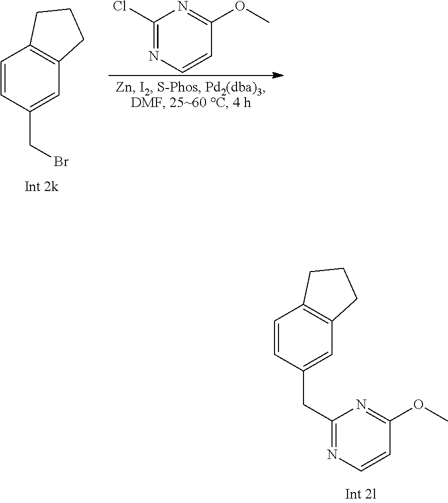

A mixture of 5-(bromomethyl)-2,3-dihydro-1H-indene, Int 2k, (100 mg, 474 umol, 1.00 eq), iodine (24.0 mg, 94.7 umol, 19.1 uL, 0.200 eq) and zinc (92.9 mg, 1.42 mmol, 3.00 eq) in dimethyl formamide (2 mL) was stirred at 25° C. for 1 h. Then 2-chloro-4-methoxypyrimidine (68.5 mg, 474 umol, 1.00 eq), dicyclohexyl-(2-(2,6-dimethoxyphenyl)phenyl)phosphane (S-Phos) (38.9 mg, 94.7 umol, 0.20 eq) and tris(dibenzylidenethyl acetoacetate)dipalladium(O) (43.38 mg, 47.37 umol, 0.1 eq) were added and the mixture was stirred at 60° C. for 3 h. The mixture was diluted with ethyl acetate (5 mL) and washed with brine (3×2 mL). The organic layer was concentrated to give a residue. The residue was purified by prep-TLC (SiO₂, petroleum ether/ethyl acetate=5/1) to afford 2-((2,3-dihydro-1H-inden-5-yl)methyl)-4-methoxypyrimidine, Int 21, (60.0 mg, 249 umol, 52% yield) as yellow oil. ¹H NMR (400 MHz, DMSO-d₆) δ=8.41 (d, J=6.0 Hz, 1H), 7.15 (s, 1H), 7.13-7.10 (m, 1H), 7.07-7.03 (m, 1H), 6.75 (d, J=6.0 Hz, 1H), 4.02 (s, 2H), 3.89 (s, 3H), 2.85-2.72 (m, 4H), 1.97 (quin, J=7.2 Hz, 2H). MS (ESI) m/z 241.2 [M+H]⁺

### Step E. Preparation of Int 2m

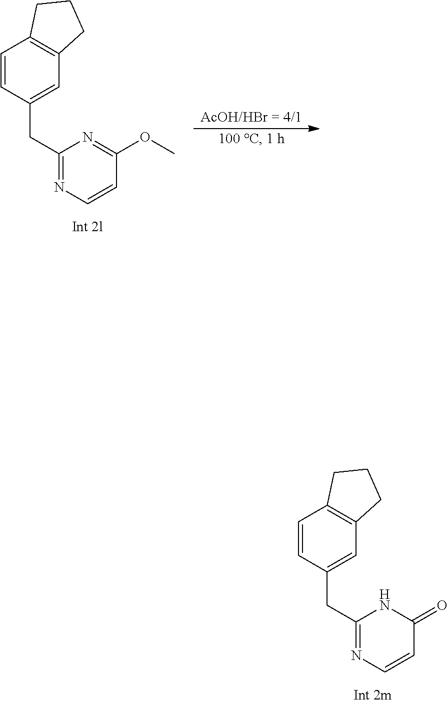

2-((2,3-dihydro-1H-inden-5-yl)methyl)-4-methoxypyrimidine, Int 21, (60.0 mg, 250 umol, 1.00 eq) in acetic acid (1.00 mL) and hydrogen bromide (0.200 mL, 48% purity) was stirred at 100° C. for 2 h. The reaction mixture was concentrated to give a residue. The residue was diluted with saturated sodium sulfate (2 mL) and extracted with ethyl acetate (3×1 mL). The organic layer was concentrated to give 2-((2,3-dihydro-1H-inden-5-yl)methyl)pyrimidin-4(3H)-one, Int 2m, (20.0 mg, crude) as a yellow solid. ¹H NMR (400 MHz, DMSO-d₆) δ=7.81 (d, J=6.8 Hz, 1H), 7.18-7.12 (m, 2H), 7.08-7.03 (m, 1H), 6.14 (d, J=6.8 Hz, 1H), 3.78 (s, 2H), 2.80 (q, J=7.2 Hz, 4H), 2.04-1.93 (m, 2H). MS (ESI) m/z 227.1 [M+H]⁺

### Step F. Preparation of Int 2n

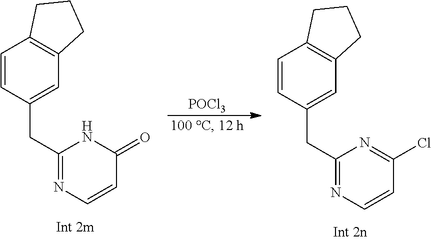 [SMILES: O=c1ccnc(Cc2ccc3c(c2)CCC3)[nH]1 ; "POCl3" ; "100 °C, 12 h" ; Clc1ccnc(Cc2ccc3c(c2)CCC3)n1]

2-((2,3-dihydro-1H-inden-5-yl)methyl)pyrimidin-4(3H)-one, Int 2m, (20.0 mg, 88.4 umol, 1 eq) in phosphorus oxychloride (1.00 mL) was stirred at 90° C. for 1 h. The mixture was cooled to 25° C., poured into saturated sodium bicarbonate (5 mL) and extracted with ethyl acetate (3×1 mL). The organic layer was concentrated to give 4-chloro-2-((2,3-dihydro-1H-inden-5-yl)methyl)pyrimidine, Int 2n, (20 mg, crude) as a yellow solid. MS (ESI) m/z 245.1 [M+H]⁺

### Step G. Preparation of Compound IG-19

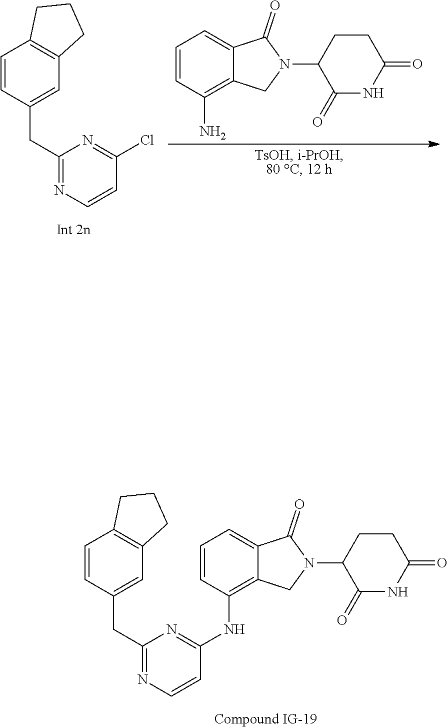

To a solution of 4-chloro-2-((2,3-dihydro-1H-inden-5-yl)methyl)pyrimidine, Int 2n, (20.0 mg, 81.7 umol, 1.00 eq) in isopropanol (1.0 mL) was added 3-(4-amino-1-oxoisoindolin-2-yl)piperidine-2,6-dione (21.1 mg, 81.7 umol, 1 eq) and p-toluenesulfonic acid (3.05 mg, 106 umol, 0.2 eq). Then the mixture was stirred at 90° C. for 2 h. The reaction mixture was concentrated to give a residue. The residue was triturated with sodium bicarbonate (2 mL) and filtered. The filter cake was purified by prep-HPLC (column: YMC-Actus Triart C₁₈ 150*30 mm*7 um; mobile phase: [water (FA)-ACN]; B %: 18%-38%, 10 min) to afford 3-(4-((2-((2,3-dihydro-1H-inden-5-yl)methyl)pyrimidin-4-yl)amino)-1-oxoisoindolin-2-yl)piperidine-2,6-dione, Compound IG-19, (31.36 mg, 62.3 umol, 76% yield, 93% purity, formate) as a white solid. ¹H NMR (400 MHz, DMSO-d₆) δ=11.00 (s, 1H), 9.31 (s, 1H), 8.23 (d, J=6.0 Hz, 1H), 7.97 (dd, J=1.6, 7.2 Hz, 1H), 7.51-7.41 (m, 2H), 7.15-7.08 (m, 2H), 7.01 (d, J=8.4 Hz, 1H), 6.67 (d, J=6.0 Hz, 1H), 5.13 (dd, J=5.2, 13.2 Hz, 1H), 4.50-4.32 (m, 2H), 3.93 (s, 2H), 2.99-2.85 (m, 1H), 2.79 (dt, J=3.2, 7.2 Hz, 4H), 2.61-2.56 (m, 1H), 2.29 (br dd, J=4.4, 13.2 Hz, 1H), 2.04-1.92 (m, 3H). MS (ESI) m/z 245.1 [M+H]⁺

### Example 10

### Synthesis of 3-(4-((2-((2,3-dihydro-1H-inden-5-yl)methyl)pyrimidin-4-yl)amino)-5-hydroxy-1-oxoisoindolin-2-yl)piperidine-2,6-dione (Compound IG-21)

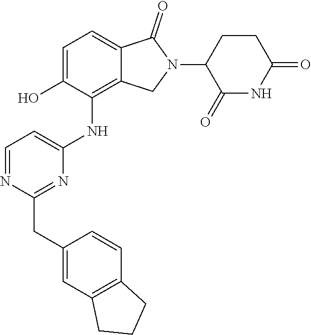 [SMILES: O=C1CCC(N2Cc3c(ccc(O)c3Nc3ccnc(Cc4ccc5c(c4)CCC5)n3)C2=O)C(=O)N1]

### Step A. Preparation of Int 20

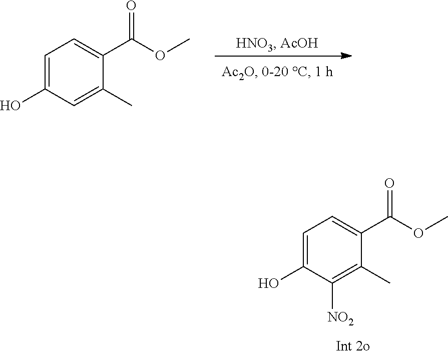

A solution of nitric acid (3.50 g, 36.1 mmol, 2.50 mL, 65% purity, 1.20 eq) and acetic acid (2.71 g, 45.1 mmol, 2.58 mL, 1.50 eq) was slowly added to a well-stirred solution of methyl 4-hydroxy-2-methylbenzoate (5.00 g, 30.1 mmol, 0.00 eq) in acetic oxide (50.0 mL) over the course of 0.5 h at 0° C. The reaction was stirred at 20° C. for 0.5 h. The reaction mixture are added to the ice water and filtered to give the filter cake. The filter cake was purified by flash silica gel chromatography (ISCO®; 80 g SepaFlash® Silica Flash Column, Eluent of 0~30% ethyl acetate/petroleum ether gradient @100 mL/min) to give methyl 4-hydroxy-2-methyl-3-nitrobenzoate, Int 2o, (1.50 g, 7.10 mmol, 24% yield) as a yellow solid. ¹H NMR (400 MHz, DMSO-d₆) δ=11.78 (s, 1H), 7.89 (d, J=8.8 Hz, 1H), 7.00 (d, J=8.8 Hz, 1H), 3.80 (s, 3H), 2.37 (s, 3H).

### Step B. Preparation of Int 2p

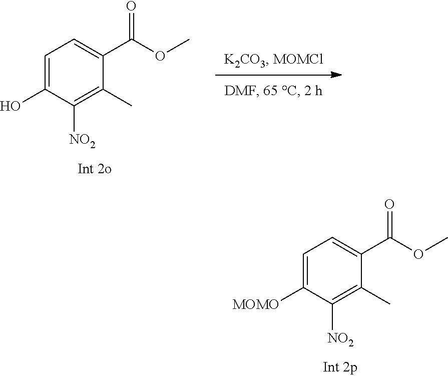

To a solution of methyl 4-hydroxy-2-methyl-3-nitrobenzoate, Int 2n, (1.35 g, 6.39 mmol, 1.00 eq) in dimethyl formamide (20.0 mL) was added potassium carbonate (3.53 g, 25.6 mmol, 4.00 eq) and chloro(methoxy)methane (1.03 g, 12.8 mmol, 971 uL, 2.00 eq). The mixture was stirred at 65° C. for 2 h. The reaction solution was poured into sodium bicarbonate solution (20.0 mL) and extracted with ethyl acetate (3×20.0 mL). After washed with an aqueous solution of sodium chloride, it was dried over anhydrous sodium sulfate and filtered. The filtrate was concentrated to give a residue. The residue was purified by flash silica gel chromatography (ISCO®; 20 g SepaFlash® Silica Flash Column, Eluent of 0~30% ethyl acetate/petroleum ether gradient @100 mL/min) to give methyl 4-(methoxymethoxy)-2-methyl-3-nitrobenzoate, Int 2p, (1.30 g, 5.09 mmol, 80% yield) as a white solid. ¹H NMR (400 MHz, DMSO-d₆) δ=8.01 (d, J=8.9 Hz, 1H), 7.34 (d, J=8.9 Hz, 1H), 5.40 (s, 2H), 3.83 (s, 3H), 3.36 (s, 3H), 2.39 (s, 3H).

### Step C. Preparation of Int 2q

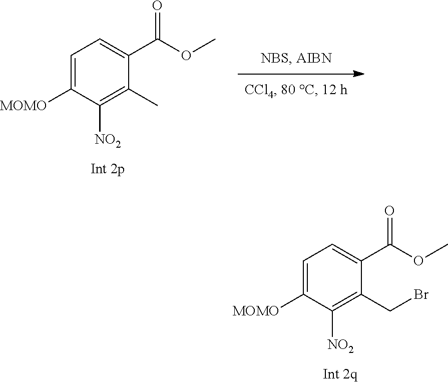

To a solution of methyl 4-(methoxymethoxy)-2-methyl-3-nitrobenzoate, Int 2p, (1.20 g, 4.70 mmol, 1.00 eq) in perchloromethane (20.0 mL) was added N-bromosuccinimide (1.00 g, 5.64 mmol, 1.20 eq) and 2,2-azobisisobutyronitrile (386 mg, 2.35 mmol, 0.50 eq). The mixture was stirred at 80° C. for 12 h. The reaction mixture was concentrated under reduced pressure to give a residue. The residue was purified by column chromatography (SiO₂, petroleum ether/ethyl acetate=30/1 to 20/1) to give methyl 2-(bromomethyl)-4-(methoxymethoxy)-3-nitrobenzoate, Int 2q, (500 mg, 1.50 mmol, 32% yield) as a yellow solid. ¹H NMR (400 MHz, DMSO-d₆) δ=8.12 (d, J=9.2 Hz, 1H), 7.52 (d, J=9.2 Hz, 1H), 5.44 (s, 2H), 4.83 (s, 2H), 3.88 (s, 3H), 3.38 (s, 3H).

### Step D. Preparation of Int 2r

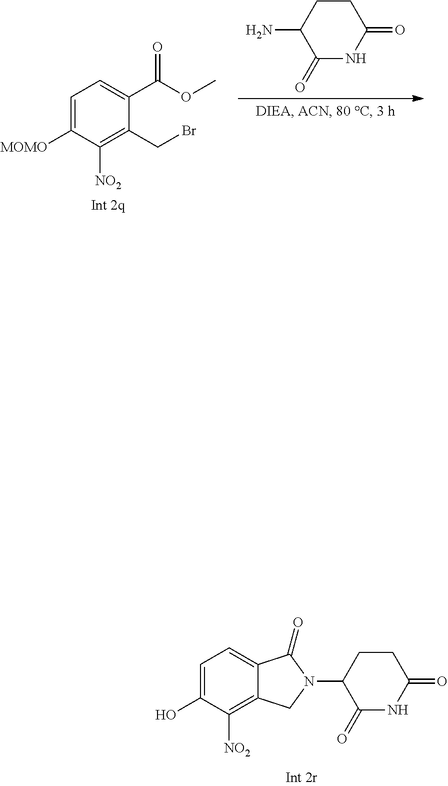

To a solution of methyl 2-(bromomethyl)-4-(methoxymethoxy)-3-nitrobenzoate, Int 2q, (500 mg, 1.50 mmol, 1.00 eq) in acetonitrile (10.0 mL) was added N,N-diisopropylethylamine (387 mg, 2.99 mmol, 521 uL, 2.00 eq) and 3-aminopiperidine-2,6-dione (211 mg, 1.65 mmol, 1.10 eq). The mixture was stirred at 80° C. for 3 h. The reaction mixture was concentrated under reduced pressure to give a residue. The residue was purified by flash silica gel chromatography (ISCO®; 12 g SepaFlash® Silica Flash Column, eluent of 0~80% ethyl acetate/petroleum ether gradient @100 mL/min) to give 3-(5-hydroxy-4-nitro-1-oxoisoindolin-2-yl)piperidine-2,6-dione, Int 2r, (120 mg, 393 umol, 26% yield) as a blue solid. ¹H NMR (400 MHz, DMSO-d₆) δ=10.98 (s, 1H), 7.87 (d, J=8.4 Hz, 1H), 7.28 (d, J=8.4 Hz, 1H), 5.08 (dd, J=5.0, 13.3 Hz, 1H), 4.73-4.67 (m, 1H), 4.62-4.55 (m, 1H), 2.95-2.83 (m, 1H), 2.58 (br d, J=15.8 Hz, 1H), 2.46-2.42 (m, 1H), 1.99-1.97 (m, 1H).

### Step E. Preparation of Int 2s

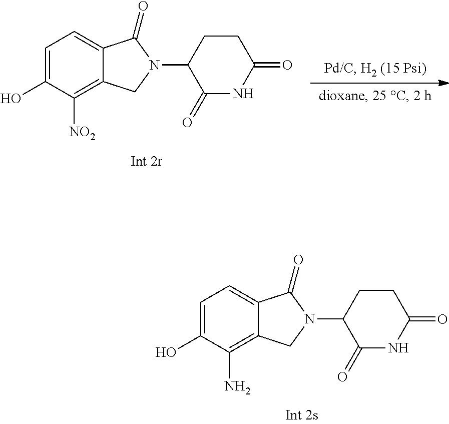 [SMILES: O=C1CCC(N2Cc3c(ccc(O)c3[N+](=O)[O-])C2=O)C(=O)N1 ; Nc1c(O)ccc2c1CN(C1CCC(=O)NC1=O)C2=O]

To a solution of 3-(5-hydroxy-4-nitro-1-oxoisoindolin-2-yl)piperidine-2,6-dione, Int 2r, (120 mg, 393 umol, 1.00 eq) in dioxane (10.0 mL) was added palladium on carbon (10%, 0.05 g) under nitrogen atmosphere. The suspension was degassed and purged with hydrogen for 3 times. The mixture was stirred under hydrogen (15 Psi) at 25° C. for 2 h. The reaction mixture was filtered. The filtrate was concentrated to give a residue. The residue was triturated with acetonitrile (5 mL) at 25° C. for 10 min and filtered to give 3-(4-amino-5-hydroxy-1-oxoisoindolin-2-yl)piperidine-2,6-dione, Int 2s, (50.0 mg, 182 umol, 46% yield) as a blue solid. ¹H NMR (400 MHz, DMSO-d₆) δ=10.96 (br s, 1H), 9.99-9.53 (m, 1H), 6.89-6.83 (m, 1H), 6.82-6.77 (m, 1H), 5.05 (br dd, J=5.2, 13.3 Hz, 1H), 4.77 (br s, 2H), 4.22-4.03 (m, 2H), 2.95-2.84 (m, 1H), 2.60 (br d, J=17.2 Hz, 1H), 2.32-2.19 (m, 1H), 2.03-1.96 (m, 1H).

### Step F. Preparation of Compound IG-21

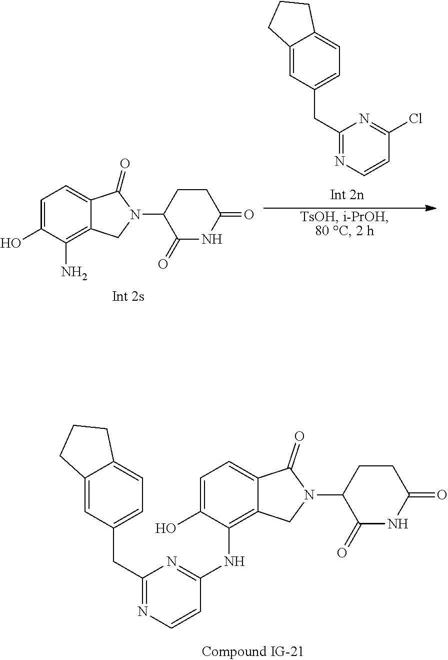 [SMILES: Nc1c(O)ccc2c1CN(C1CCC(=O)NC1=O)C2=O ; Clc1ccnc(Cc2ccc3c(c2)CCC3)n1 ; "TsOH, i-PrOH,80 °C, 2 h" ; O=C1CCC(N2Cc3c(ccc(O)c3Nc3ccnc(Cc4ccc5c(c4)CCC5)n3)C2=O)C(=O)N1]

To a solution of 4-chloro-2-((2,3-dihydro-1H-inden-5-yl)methyl)pyrimidine, Int 2n, (35.6 mg, 145 umol, 1.00 eq) in isopropanol (1.00 mL) was added para-toluenesulfonic acid (5.00 mg, 29.1 umol, 0.20 eq) and 3-(4-amino-5-hydroxy-1-oxoisoindolin-2-yl) piperidine-2,6-dione, Int 2s, (40.0 mg, 145 umol, 1.00 eq). The mixture was stirred at 80° C. for 2 h. The reaction mixture was concentrated under reduced pressure to give a residue. The residue was diluted with sodium bicarbonate (5.00m L) and filtered to give a residue. The residue was purified by prep-HPLC(column: Phenomenex luna C18 150*25 mm*10 um; mobile phase: [water(FA)-ACN]; B %: 5%-35%, 10 min) and further purified by prep-HPLC (column: Phenomenex luna C18 150*25 mm*10 um; mobile phase: [water(FA)-ACN]; B %: 5%-35%, 10 min) to give 3-(4-((2-((2,3-dihydro-1H-inden-5-yl)methyl)pyrimidin-4-yl)amino)-5-hydroxy-1-oxoisoindolin-2-yl)piperidine-2,6-dione, Compound IG-21, (3.91 mg, 7.68 umol, 7.4% yield, 95% purity) as a yellow solid. ¹H NMR (400 MHz, DMSO-d₆) δ=10.91 (s, 1H), 10.64-10.33 (m, 1H), 8.92 (s, 1H), 8.12 (d, J=5.9 Hz, 1H), 7.48 (d, J=8.1 Hz, 1H), 7.09-7.04 (m, 3H), 6.96 (br d, J=7.4 Hz, 1H), 6.45-6.25 (m, IH), 5.04 (dd, J=5.2, 13.3 Hz, 1H), 4.23 (s, 2H), 3.84 (s, 2H), 2.92-2.82 (m, 1H), 2.80-2.74 (m, 4H), 2.59 (br d, J=7.3 Hz, 1H), 2.28-2.20 (m, 1H), 2.00-1.93 (m, 2H), 1.90-1.83 (in, 1H). MS (ESI) m/z 534.3 [M+H]⁺

### Example 11

### Synthesis of 3-(4-((2-((2,3-dihydro-1H-inden-5-yl)methyl)pyrimidin-4-yl)amino)-6-hydroxy-1-oxoisoindolin-2-yl)piperidine-2,6-dione (Compound IG-22)

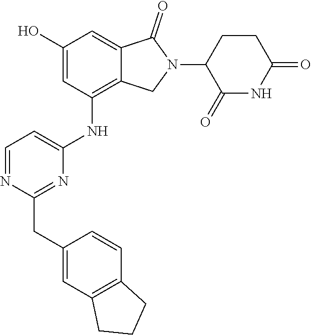 [SMILES: O=C1CCC(N2Cc3c(Nc4ccnc(Cc5ccc6c(c5)CCC6)n4)cc(O)cc3C2=O)C(=O)N1]

### Step A. Preparation of Int 2t

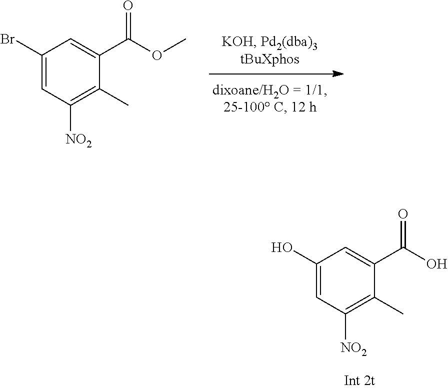

A mixture of methyl 5-bromo-2-methyl-3-nitrobenzoate (5.00 g, 18.2 mmol, 5.00 mL, 1.00 eq) and potassium hydroxide (4.09 g, 73.0 mmol, 4.00 eq) in dioxane (30.0 mL) and water (30.0 mL) was stirred at 25° C. for 5 min. Then tris(dibenzylideneacetone)dipalladium(O) (1.67 g, 1.82 mmol, 0.100 eq) and ditert-butyl-(2-(2,4,6-triisopropylphenyl)phenyl)phosphane (775 mg, 1.82 mmol, 0.100 eq) were added to the reaction mixture. The mixture was stirred at 100° C. for 12 h under nitrogen atmosphere. The reaction mixture was concentrated under reduced pressure to give a residue. The residue was purified by column chromatography (SiO₂, petroleum ether/ethyl acetate=100/1 to 10/1) to afford 5-hydroxy-2-methyl-3-nitrobenzoic acid, Int 2t, (1.80 g, 9.13 mmol, 50% yield) as a yellow solid. ¹H NMR (400 MHz, DMSO-d₆) δ=13.27 (br s, 1H), 10.48 (br s, 1H), 7.40 (d, J=2.4 Hz, 1H), 7.34 (d, J=2.4 Hz, 1H), 2.37 (s, 3H).

### Step B. Preparation of Int 2u

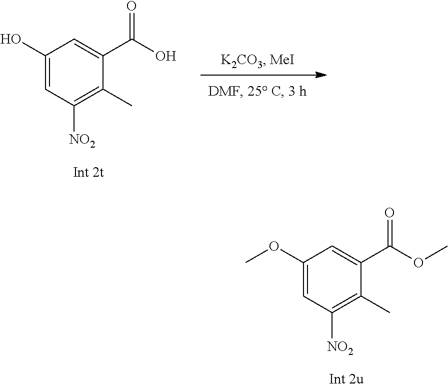

To a solution of 5-hydroxy-2-methyl-3-nitrobenzoic acid, Int 2t, (1.80 g, 9.13 mmol, 1.00 eq) in dimethylformamide (20.0 mL) was added iodomethane (5.18 g, 36.5 mmol, 2.27 mL, 4.00 eq) and potassium carbonate (5.05 g, 36.5 mmol, 4.00 eq). The mixture was stirred at 25° C. for 3 h. The reaction mixture was diluted with water (100 mL) and extracted with ethyl acetate (200 mL). The combined organic layers were washed with brine (50.0 mL) dried over anhydrous sodium sulfate, filtered and concentrated under reduced pressure to give a residue. The residue was purified by column chromatography (SiO₂, petroleum ether/ethyl acetate=100/1 to 10/1) to afford 3-methyl 5-methoxy-2-methyl-3-nitrobenzoate, Int 2u, (1.80 g, 7.59 mmol, 83% yield, 95% purity) as a white solid. ¹H NMR (400 MHz, DMSO-d₆) δ=7.66 (d, J=2.8 Hz, 1H), 7.54 (d, J=2.8 Hz, 1H), 3.88 (s, 3H), 3.86 (s, 3H), 2.37 (s, 3H).

### Step C. Preparation of Int 2v

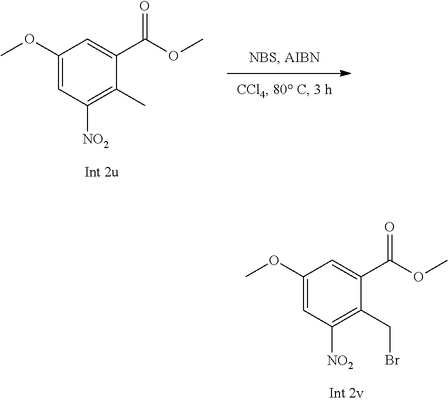

To a solution of methyl 5-methoxy-2-methyl-3-nitro-benzoate, Int 2u, (1.00 g, 4.44 mmol, 1.00 eq) in carbon tetrachloride (15.0 mL) was added N-bromosuccinimide (948 mg, 5.33 mmol, 1.20 eq) and 2,2-azobisisobutyronitrile (72.9 mg, 444 umol, 0.100 eq). The mixture was stirred at 80° C. for 3 h. The reaction mixture was concentrated under reduced pressure to give a residue. The residue was purified by column chromatography (SiO₂, petroleum ether/ethyl acetate=100/1 to 5/1) to afford methyl 2-(bromomethyl)-5-methoxy-3-nitrobenzoate, Int 2v, (1.20 g, 3.95 mmol, 88% yield) as a white solid. ¹H NMR (400 MHz, DMSO-d₆) δ=7.76 (d, J=2.8 Hz, 1H), 7.63 (d, J=2.8 Hz, 1H), 4.95 (s, 2H), 3.92 (s, 3H), 3.91 (s, 3H).

### Step D. Preparation of Int 2w

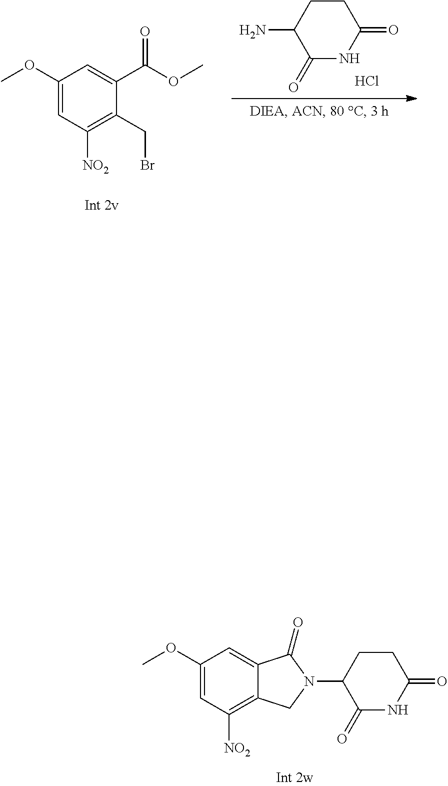

A mixture of methyl 2-(bromomethyl)-5-methoxy-3-nitrobenzoate, Int 2v, (1.20 g, 3.95 mmol, 1.00 eq), 3-aminopiperidine-2,6-dione;hydrochloride (714 mg, 4.34 mmol, 1.10 eq, hydrochloric acid), diisopropylethylamine (1.02 g, 7.89 mmol, 1.37 mL, 2.00 eq) in acetonitrile (15.0 mL) was degassed and purged with nitrogen for 3 times, and then the mixture was stirred at 80° C. for 3 h under nitrogen atmosphere. The reaction mixture was concentrated under reduced pressure to give a residue. The crude product was triturated with water (50.0 mL) and filtered to afford 3-(6-methoxy-4-nitro-1-oxoisoindolin-2-yl)piperidine-2,6-dione, Int 2w, (1.20 g, 3.76 mmol, 95% yield) as a purple solid. ¹H NMR (400 MHz, DMSO-d₆) δ=11.03 (s, 1H), 7.94 (d, J=2.4 Hz, 1H), 7.72 (d, J=2.4 Hz, 1H), 5.18 (dd, J=5.1, 13.2 Hz, 1H), 4.85-4.67 (m, 2H), 3.98 (s, 3H), 2.98-2.86 (m, 1H), 2.61 (br d, J=18.8 Hz, 1H), 2.56-2.52 (m, 1H), 2.07-1.98 (m, 1H).

### Step E. Preparation of Int 2x

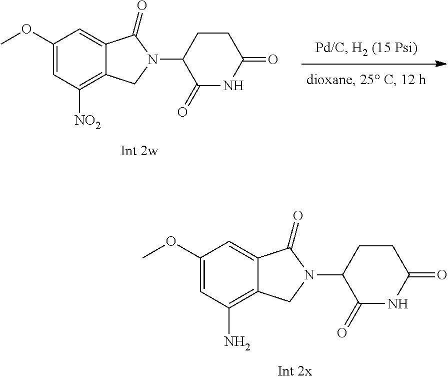 [SMILES: COc1cc2c(c([N+](=O)[O-])c1)CN(C1CCC(=O)NC1=O)C2=O ; COc1cc(N)c2c(c1)C(=O)N(C1CCC(=O)NC1=O)C2]

To a solution of 3-(6-methoxy-4-nitro-1-oxo-isoindolin-2-yl)piperidine-2,6-dione, Int 2w, (1.60 g, 5.01 mmol, 1.00 eq) in dioxane (20.0 mL) was added palladium/carbon (200 mg, 10.0% purity) under nitrogen atmosphere. The suspension was degassed and purged with hydrogen for 3 times. The mixture was stirred under hydrogen atmosphere (15 Psi) at 25° C. for 12 h. The mixture was filtered and the filtrated was concentrated under reduced pressure to afford 3-(4-amino-6-methoxy-1-oxoisoindolin-2-yl)piperidine-2,6-dione, Int 2x, (1.30 g, 2.70 mmol, 53% yield, 60% purity) as a green solid. ¹H NMR (400 MHz, DMSO-d₆) δ=10.98 (br s, 1H), 6.47 (d, J=2.0 Hz, 1H), 6.37 (d, J=2.4 Hz, 1H), 5.45 (s, 2H), 5.11-5.09 (m, 1H), 4.20 (d, J=15.6 Hz, 2H), 3.78 (s, 3H), 2.91 (br d, J=4.0 Hz, 1H), 2.62 (br s, 1H), 2.34-2.32 (m, 1H), 2.01 (br d, J=7.2 Hz, 1H).

### Step F. Preparation of Int 2y

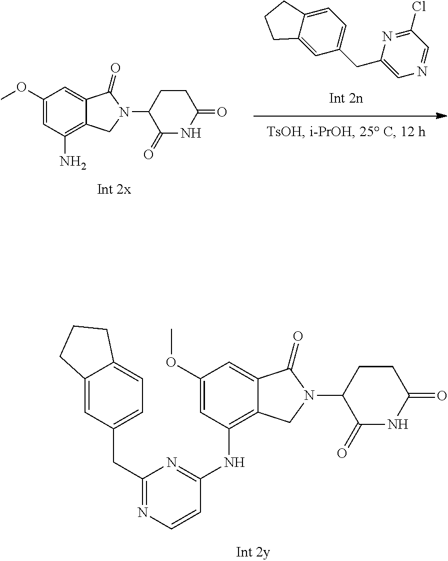 [SMILES: COc1cc(N)c2c(c1)C(=O)N(C1CCC(=O)NC1=O)C2 ; Clc1cncc(Cc2ccc3c(c2)CCC3)n1 ; COc1cc(Nc2ccnc(Cc3ccc4c(c3)CCC4)n2)c2c(c1)C(=O)N(C1CCC(=O)NC1=O)C2]

To a solution of 3-(4-amino-6-methoxy-1-oxo-isoindolin-2-yl)piperidine-2,6-dione, Int 2x, (700 mg, 2.42 mmol, 1.00 eq) in isopropyl alcohol (5.00 mL) was added p-toluene sulfonic acid (83.3 mg, 484 umol, 0.200 eq) and 4-chloro-2-(indan-5-ylmethyl)pyrimidine, Int 2n, (665 mg, 2.72 mmol, 1.12 eq). The mixture was stirred at 25° C. for 12 h. The reaction mixture was concentrated under reduced pressure to give a residue. The residue was purified by reversed-phase HPLC(column: Waters Xbridge 150*25 mm*5 um; mobile phase: [water(NH₄HCO₃)-ACN]; B %: 32%-62%, 9 min). The desired fraction was lyophilized to afford 3-(4-amino-6-methoxy-1-oxoisoindolin-2-yl)piperidine-2,6-dione, Int 2y, (100 mg, 200 umol, 8% yield) as a white solid. ¹H NMR (400 MHz, DMSO-d₆) δ=11.01 (s, 1H), 9.27 (s, 1H), 8.26 (d, J=6.0 Hz, 1H), 7.80 (d, J=2.0 Hz, 1H), 7.15-7.09 (m, 2H), 7.04-6.99 (m, 2H), 6.72 (d, J=6.0 Hz, 1H), 5.18-5.10 (m, 1H), 4.40-4.28 (m, 2H), 3.95 (s, 2H), 3.82 (s, 3H), 2.79 (br t, J=7.2 Hz, 4H), 2.39 (br s, 3H), 2.02-1.95 (m, 3H). MS (ESI) m/z 498.2 [M+H]⁺

### Step G. Preparation of Compound IG-22

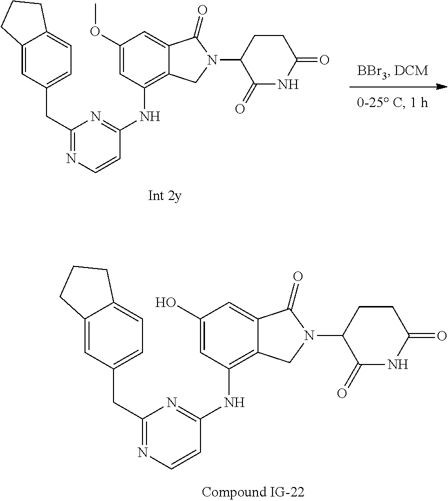 [SMILES: COc1cc(Nc2ccnc(Cc3ccc4c(c3)CCC4)n2)c2c(c1)C(=O)N(C1CCC(=O)NC1=O)C2 ; O=C1CCC(N2Cc3c(Nc4ccnc(Cc5ccc6c(c5)CCC6)n4)cc(O)cc3C2=O)C(=O)N1]

To a solution of 3-(4-amino-6-methoxy-1-oxoisoindolin-2-yl)piperidine-2,6-dione, Int 2y, (100 mg, 201 umol, 1.00 eq) in dichloromethane (3.00 mL) was added boron tribromide (504 mg, 2.01 mmol, 194 uL, 10.0 eq) at 0° C. The mixture was stirred at 25° C. for 1 h. The reaction mixture was concentrated under reduced pressure to give a residue. The residue was purified by reversed-phase HPLC(column: Waters Xbridge 150*25 mm*5 um; mobile phase: [water(NH₄HCO₃)-ACN]; B %: 20%-50%, 5 min) to afford 3-(4-((2-((2,3-dihydro-1H-inden-5-yl)methyl)pyrimidin-4-yl)amino)-6-hydroxy-1-oxoisoindolin-2-yl)piperidine-2,6-dione, Compound IG-22, (14.4 mg, 28.0 umol, 13% yield, 94% purity) as a white solid. ¹H NMR (400 MHz, DMSO-d₆) δ=10.99 (s, 1H), 9.86 (s, 1H), 9.21 (s, 1H), 8.23 (d, J=6.0 Hz, 1H), 7.56 (d, J=2.0 Hz, 1H), 7.14 (s, 1H), 7.12-7.08 (m, 1H), 7.06-7.01 (m, 1H), 6.85 (d, J=2.0 Hz, 1H), 6.66 (d, J=6.0 Hz, 1H), 5.09 (dd, J=5.2, 13.2 Hz, 1H), 4.34-4.23 (m, 2H), 3.94 (s, 2H), 2.95-2.86 (m, 1H), 2.79 (dt, J=3.2, 7.2 Hz, 4H), 2.59 (br d, J=2.8 Hz, 1H), 2.26 (dq, J=4.2, 13.2 Hz, 1H), 2.01-1.93 (m, 3H). MS (ESI) m/z 484.2 [M+H]⁺

### Example 12

### Synthesis of 3-(4-((2-((2,3-dihydro-1H-inden-5-yl)methyl)pyrimidin-4-yl)amino)-7-hydroxy-1-oxoisoindolin-2-yl)piperidine-2,6-dione (Compound IG-23)

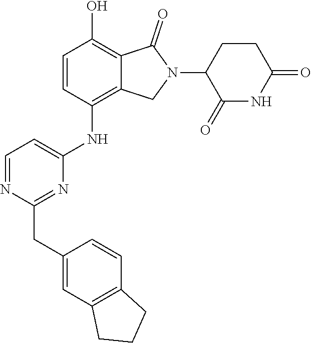 [SMILES: O=C1CCC(N2Cc3c(Nc4ccnc(Cc5ccc6c(c5)CCC6)n4)ccc(O)c3C2=O)C(=O)N1]

### Step A. Preparation of Int 2z

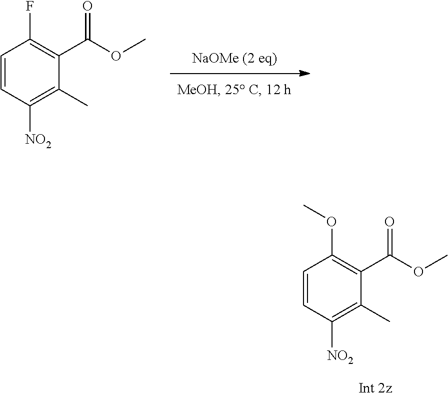 [SMILES: COC(=O)c1c(F)ccc([N+](=O)[O-])c1C ; "NaOMe (2 eq)" ; COC(=O)c1c(OC)ccc([N+](=O)[O-])c1C]

Methyl 6-fluoro-2-methyl-3-nitro-benzoate (1.00 g, 4.69 mmol, 1.00 eq) was added into a solution of sodium methoxide (507 mg, 9.38 mmol, 2.00 eq) in methanol (10.0 mL). The mixture was stirred at 25° C. for 12 h. The mixture was concentrated to give a residue. The mixture was concentrated to afford methyl 6-methoxy-2-methyl-3-nitro-benzoate, Int 2z, (980 mg, 4.35 mmol, 93% yield) as a yellow oily substance. ¹H NMR (400 MHz, DMSO-d₆) δ=8.15 (d, J=92 Hz, 1H), 7.19 (d, J=9.2 Hz, 1H), 3.88 (d, J=14.8 Hz, 6H), 2.36 (s, 3H).

### Step B. Preparation of Int 3a

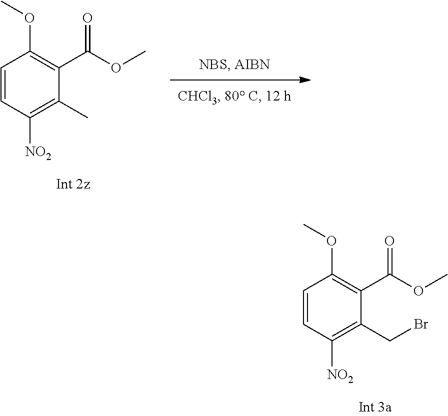

To a solution of methyl 6-methoxy-2-methyl-3-nitro-benzoate, Int 2z, (700 mg, 3.11 mmol, 1.00 eq) in trichloromethane (10.0 mL) was added 2,2-azobisisobutyronitrile (153 mg, 933 umol, 0.300 eq) and N-bromosuccinimide (1.11 g, 6.22 mmol, 2.00 eq). The mixture was stirred at 80° C. for 12 h. The mixture was concentrated to give a residue. The residue was purified by column chromatography (SiO2, Petroleum ether/ethyl acetate=10/1). The desired fraction was collected and concentrated to afford methyl 2-(bromomethyl)-6-methoxy-3-nitro-benzoate, Int 3a, (600 mg, 1.97 mmol, 63% yield) as a yellow solid. ¹H NMR (400 MHz, DMSO-d₆) δ=8.26 (d, J=9.2 Hz, 1H), 7.35 (d, J=9.2 Hz, 1H), 4.70 (s, 2H), 3.93 (s, 3H), 3.89 (s, 3H).

### Step C. Preparation of Int 3b

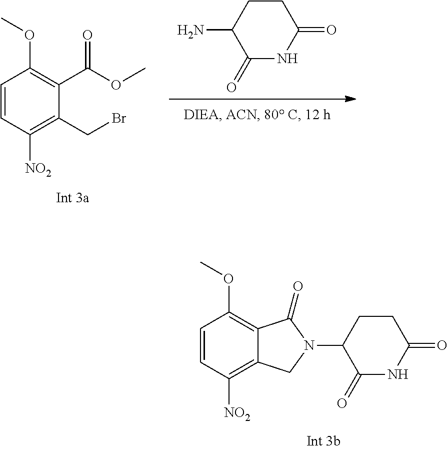

To a solution of methyl 2-(bromomethyl)-6-methoxy-3-nitro-benzoate, Int 3a, (1.00 g, 3.29 mmol, 1.00 eq) in acetonitrile (10.0 mL) was added diisopropylethylamine (1.28 g, 9.87 mmol, 1.72 mL, 3.00 eq) and 3-aminopiperidine-2,6-dione;hydrochloride (541 mg, 3.29 mmol, 1.00 eq). The mixture was stirred at 80° C. for 12 h. The mixture was concentrated to give a residue. The residue was triturated with ethyl acetate (3.00 mL) and water (1.00 mL) at 25° C. for 1 h. The solid was collected by filtration and dried in vacuo to afford 3-(7-methoxy-4-nitro-1-oxo-isoindolin-2-yl)piperidine-2,6-dione, Int 3b, (920 mg, crude) as a purple solid. ¹H NMR (400 MHz, DMSO-d₆) δ=10.99 (s, 1H), 8.46 (d, J=9.2 Hz, 1H), 7.35 (d, J=9.2 Hz, 1H), 5.08 (dd, J=5.2, 13.2 Hz, 1H), 4.88-4.61 (m, 2H), 4.03 (s, 3H), 2.96-2.85 (m, 1H), 2.58 (br d, J=18.0 Hz, 1H), 2.47-2.41 (m, 1H), 2.03-1.93 (m, 1H). MS (ESI) m/z 320.1 [M+H]⁺

### Step D. Preparation of Int 3c

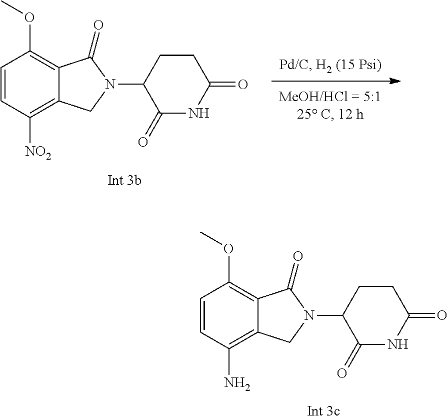 [SMILES: COc1ccc([N+](=O)[O-])c2c1C(=O)N(C1CCC(=O)NC1=O)C2 ; COc1ccc(N)c2c1C(=O)N(C1CCC(=O)NC1=O)C2]

To a solution of 3-(7-methoxy-4-nitro-1-oxo-isoindolin-2-yl)piperidine-2,6-dione, Int 3b, (0.920 g, 2.88 mmol, 1.00 eq) in methanol (10.0 mL) and hydrochloric acid (2.00 mL) was added wet palladium on carbon (100 mg, 10% purity) under nitrogen. The mixture was stirred at 25° C. for 12 h under hydrogen (15 Psi). The mixture was filtered to give filtrate that was concentrated to afford 3-(4-amino-7-methoxy-1-oxo-isoindolin-2-yl)piperidine-2,6-dione, Int 3c, (0.820 g, crude) as a black gum. ¹H NM R (400 MHz, DMSO-d₆) δ=11.01 (s, 1H), 9.26-9.08 (m, 1H), 7.62 (d, J=8.8 Hz, 1H), 7.17 (d, J=8.8 Hz, 1H), 5.07 (dd, J=5.2, 13.3 Hz, 1H), 4.55-4.48 (m, 1H), 4.43-4.36 (m, 1H), 3.88 (s, 3H), 2.96-2.87 (m, 1H), 2.62-2.57 (m, 1H), 2.48-2.40 (m, 1H), 2.03-1.96 (m, 1H). MS (ESI) m/z 290.2 [M+H]⁺

### Step E. Preparation of Int 3d

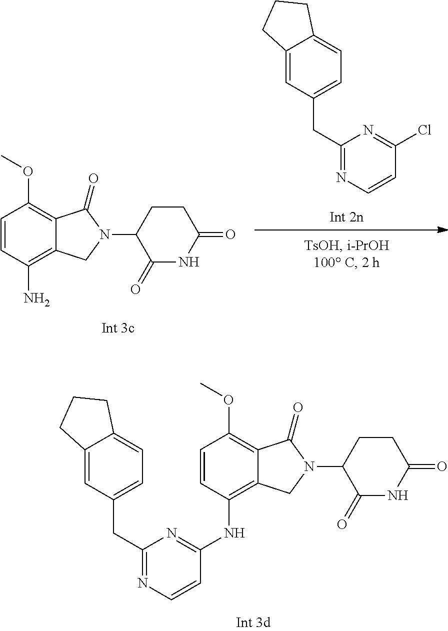

To a solution of 3-(4-amino-7-methoxy-1-oxo-isoindolin-2-yl)piperidine-2,6-dione, Int 3c, (380 mg, 1.31 mmol, 1.00 eq) and 4-chloro-2-(indan-5-ylmethyl)pyrimidine, Int 2n, (354 mg, 1.44 mmol, 1.10 eq) in isopropanol (10.0 mL) was added p-toluenesulfonic acid (45.2 mg, 263 umol, 0.200 eq). The mixture was stirred at 100° C. for 2 h. The mixture was concentrated to give a residue. The residue was purified by prep-HPLC (0.1% FA). The desired fraction was collected and lyophilized to afford 3-(4-((2-((2,3-dihydro-1H-inden-5-yl)methyl) pyrimidin-4-yl)amino)-7-methoxy-1-oxoisoindolin-2-yl)piperidine-2,6-dione, Int 3d, (266 mg, 535 umol, 41% yield) as a black solid. ¹H NMR (400 MHz, DMSO-d₆) δ=10.97 (s, 1H), 8.31 (d, J=7.2 Hz, 1H), 7.71-7.54 (m, 1H), 7.29-6.80 (m, 6H), 5.05 (dd, J=5.0, 13.4 Hz, 1H), 4.38-4.18 (m, 2H), 4.09 (br s, 2H), 3.91 (s, 3H), 2.97-2.87 (m, 1H), 2.80 (q, J=7.3 Hz, 4H), 2.57 (br s, 1H), 2.28-2.18 (m, 1H), 2.04-1.95 (m, 2H), 1.95-1.88 (m, 1H).

MS (ESI) m/z 498.2 [M+H]⁺

### Step F. Preparation of Compound IG-22

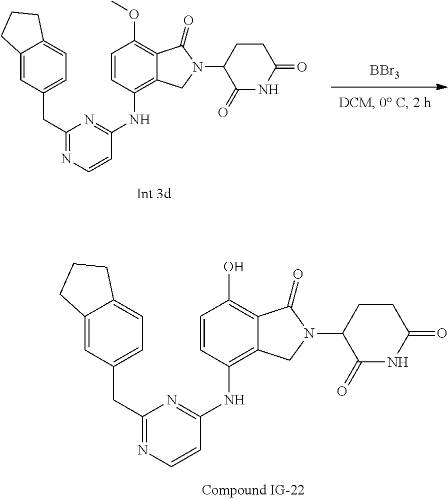 [SMILES: COc1ccc(Nc2ccnc(Cc3ccc4c(c3)CCC4)n2)c2c1C(=O)N(C1CCC(=O)NC1=O)C2 ; "BBr3" ; O=C1CCC(N2Cc3c(Nc4ccnc(Cc5ccc6c(c5)CCC6)n4)ccc(O)c3C2=O)C(=O)N1]

To a solution of 3-(4-((2-((2,3-dihydro-1H-inden-5-yl)methyl)pyrimidin-4-yl)amino)-7-methoxy-1-oxoisoindolin-2-yl)piperidine-2,6-dione, Int 3d, (80.0 mg, 161 umol, 1.00 eq) in dichloromethane (2.00 mL) was added boron tribromide (806 mg, 3.22 mmol, 310 uL, 20.0 eq) at 0° C. The mixture was stirred at 0° C. for 2 h. The mixture was diluted with water (20.0 m1) at 0° C., and filtered to give filtered cake. The solid was purified by prep-HPLC (0.1% FA). The desired fraction was collected and lyophilized to afford 3-(4-((2-((2,3-dihydro-1H-inden-5-yl)methyl)pyrimidin-4-yl)amino)-7-hydroxy-1-oxoisoindolin-2-yl)piperidine-2,6-dione, Compound IG-22, (8.22 mg, 16.7 umol, 10% yield, 98% purity) as a gray solid. ¹H NMR (400 MHz, DMSO-d₆) δ=10.96 (s, 1H), 9.88-9.70 (m, 1H), 8.17 (d, J=6.0 Hz, 1H), 7.53-7.42 (m, 1H), 7.14-7.09 (m, 2H), 6.99 (br d, J=7.5 Hz, 1H), 6.85 (d, J=8.4 Hz, 1H), 6.58-6.48 (m, 1H), 5.04 (dd, J=5.2, 13.3 Hz, 1H), 4.24 (br d, J=5.6 Hz, 2H), 3.92 (s, 2H), 2.97-2.85 (m, 1H), 2.82-2.77 (m, 4H), 2.56 (br d, J=3.6 Hz, 1H), 2.30-2.24 (m, 1H), 2.01-1.96 (m, 2H), 1.94-1.88 (m, 1H). MS (ESI) m/z 484.2 [M+H]⁺

The compounds in Table 10 were prepared in a manner similar to that described for Compound IG-22 using Int 3c and the appropriately substituted chloro-pyrimidine (c.f. Example 2).

TABLE 10

| IG No. | Structure | ¹H NMR/MS |
|---|---|---|
| 44 | 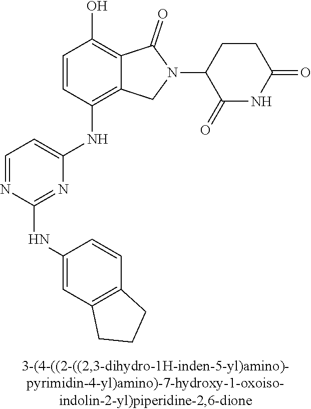 [SMILES: O=C1CCC(N2Cc3c(Nc4ccnc(Nc5ccc6c(c5)CCC6)n4)ccc(O)c3C2=O)C(=O)N1] | ¹H NMR (400 MHz, DMSO-d₆) δ = 10.93 (s, 1H), 9.77-9.56 (m, 1H), 8.93 (s, 1H), 8.85 (s, 1H), 8.20 (s, 1H), 7.94 (d, J = 5.6 Hz, 1H), 7.60-7.50 (m, 2H), 7.23 (br d, J = 8.4 Hz, 1H), 6.95 (d, J = 8.4 Hz, 1H), 6.88 (d, J = 8.8 Hz, 1H), 6.06 (d, J = 6.0 Hz, 1H), 5.01 (dd, J = 5.2, 13.1 Hz, 1H), 4.39-4.30 (m, 1H), 4.24-4.15 (m, 1H), 2.91-2.78 (m, 1H), 2.73 (br t, J = 7.2 Hz, 2H), 2.70-2.65 (m, 2H), 2.54-2.52 (m, 1H), 2.30-2.16 (m, 1H), 1.95 (quin, J = 7.2 Hz, 2H), 1.80-1.70 (m, 1H). MS (ESI) m/z 485.3 [M + H]⁺ |

### Example 13

### Synthesis of 1-(3-chloro-4-methylphenyl)-3-((2-(2,6-dioxopiperidin-3-yl)-7-hydroxy-1-oxoisoindolin-5-yl)methyl)urea (Compound IG-24)

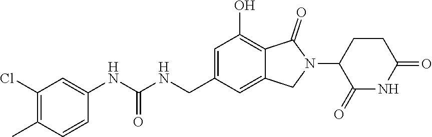 [SMILES: Cc1ccc(NC(=O)NCc2cc(O)c3c(c2)CN(C2CCC(=O)NC2=O)C3=O)cc1Cl]

### Step A. Preparation of Int 3d

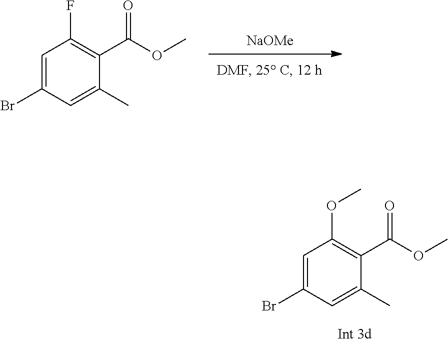

To a solution of methyl 4-bromo-2-fluoro-6-methylbenzoate (10.0 g, 40.5 mmol, 1.00 eq) in N,N-dimethylformamide (100 mL) was added sodium methanolate (10.9 g, 202 mmol, 5.00 eq), the mixture was stirred at 25° C. for 12 h. The mixture was diluted with water (200 mL) and extracted with ethyl acetate (3×100 mL). The combined organic layers were washed with brine (2×500 mL), dried over sodium sulfate and filtered. The filtrate was concentrated under reduced pressure to give a residue. The residue was purified by flash silica gel chromatography (ISCO®; 120 g SepaFlash® Silica Flash Column, Eluent of 0~10% ethyl acetate/petroleum ether @100 mL/min) to afford methyl 4-bromo-2-methoxy-6-methylbenzoate, Int 3d, (9.90 g, 38.2 mmol, 94% yield) as a white solid. ¹H NMR (400 MHz, CDCl₃) δ=6.98 (d, J=1.2 Hz, 1H), 6.91 (d, J=1.2 Hz, 1H), 3.91 (s, 3H), 3.82 (s, 3H), 2.26 (s, 3H).

### Step B. Preparation of Int 3f

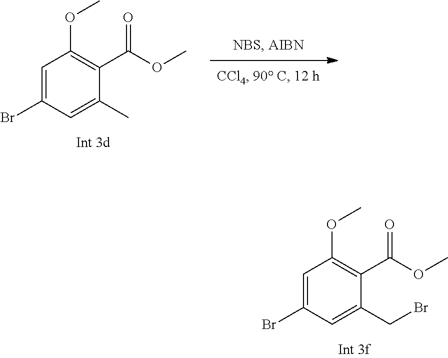

To a solution of methyl 4-bromo-2-methoxy-6-methylbenzoate, Int 3d, (9.90 g, 38.2 mmol, 1.00 eq) in perchloromethane (200 mL) was added 1-bromopyrrolidine-2,5-dione (8.16 g, 45.9 mmol, 1.20 eq) and (E)-3,3′-(diazene-1,2-diyl)bis(2-methylpropanenitrile) (627 mg, 3.82 mmol, 0.100 eq), the mixture was stirred at 90° C. for 12 h. The mixture was filtered and the filtrate was concentrated to give a residue. The residue was purified by flash silica gel chromatography (ISCO®; 120 g SepaFlash® Silica Flash Column, Eluent of 0~10% ethyl acetate/petroleum ether at 100 mL/min) to afford methyl 4-bromo-2-(bromomethyl)-6-methoxybenzoate, Int 3f, (13.0 g, crude) as colorless oil. ¹H NMR (400 MHz, CDCl₃) δ=7.18 (d, J=1.6 Hz, 1H), 7.04 (d, J=1.6 Hz, 1H), 4.43 (s, 2H), 3.95 (s, 3H), 3.85 (s, 3H).

### Step C. Preparation of Int 3g

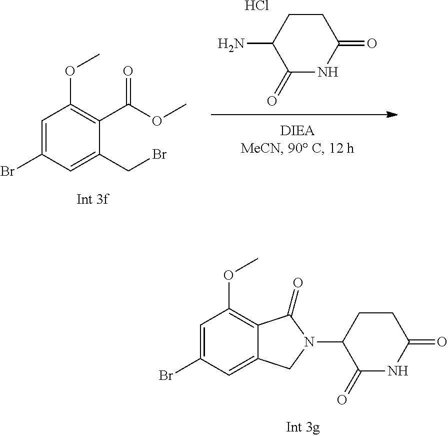 [SMILES: Cl ; COC(=O)c1c(CBr)cc(Br)cc1OC ; NC1CCC(=O)NC1=O ; COc1cc(Br)cc2c1C(=O)N(C1CCC(=O)NC1=O)C2]

To a solution of methyl 4-bromo-2-(bromomethyl)-6-methoxybenzoate, Int 3f, (17.0 g, 50.3 mmol, 1.00 eq) and 3-aminopiperidine-2,6-dione (12.4 g, 75.5 mmol, 1.50 eq, hydrochloride) in acetonitrile (150 mL) was added diisopropylethylamine (19.5 g, 151 mmol, 26.3 mL, 3.00 eq), the mixture was stirred at 90° C. for 12 h. The mixture was concentrated to give a residue. The residue was triturated with ethyl acetate/water=2/1 (50.0 mL) and filtered to afford 3-(5-bromo-7-methoxy-1-oxoisoindolin-2-yl)piperidine-2,6-dione, Int 3g, (15.0 g, 42.5 mmol, 84% yield) as a brown solid. ¹H NMR (400 MHz, DMSO-d₆) δ=10.96 (s, 1H), 7.38 (s, 1H), 7.26 (s, 1H), 5.02 (dd, J=5.2, 13.2 Hz, 1H), 4.45-4.31 (m, 1H), 4.29-4.18 (m, 1H), 3.89 (s, 3H), 2.96-2.82 (m, 1H), 2.61-2.54 (m, 1H), 2.39-2.26 (m, 1H), 2.02-1.88 (m, 1H).

### Step D. Preparation of Int 3h

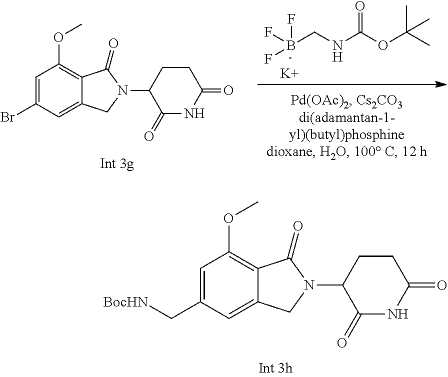

To a solution of 3-(5-bromo-7-methoxy-1-oxoisoindolin-2-yl)piperidine-2,6-dione, Int 3g, (1.00 g, 2.83 mmol, 1.00 eq) and potassium;(((tert-butoxycarbonyl)amino)methyl)trifluoroborate (872 mg, 3.68 mmol, 1.30 eq) in dioxane (20.0 mL) and water (2.00 mL) was added cesium carbonate (1.85 g, 5.66 mmol, 2.00 eq), bis(1-adamantyl)-butyl-phosphane (406 mg, 1.13 mmol, 0.400 eq) and palladium(II) acetate (127 mg, 566 umol, 0.200 eq). The mixture was stirred at 100° C. for 12 h under nitrogen atmosphere. The mixture was concentrated under reduced pressure to give a residue. The residue was purified by flash silica gel chromatography (ISCO®; 12 g SepaFlash® Silica Flash Column, Eluent of 0~100% ethyl acetate/petroleum ether @100 mL/min) to afford tert-butyl ((2-(2,6-dioxopiperidin-3-yl)-7-methoxy-1-oxoisoindolin-5-yl)methyl)carbamate, Int 3h, (650 mg, 1.27 mmol, 15% yield, 79% purity) as a yellow solid. ¹H NMR (400 MHz, DMSO-d₆) δ=10.94 (br s, 1H), 7.47 (brt, J=5.6 Hz, 1H), 6.94 (d, J=15.2 Hz, 2H), 5.01 (dd, J=5.2, 13.3 Hz, 1H), 4.37-4.17 (m, 4H), 3.84 (s, 3H), 2.90 (ddd, J=5.2, 13.8, 17.4 Hz, 1H), 2.62-2.54 (m, 2H), 2.45-2.34 (m, 1H), 1.40 (s, 9H). MS (ESI) m/z 404.1 [M+H]⁺

### Step E. Preparation of Int 3i

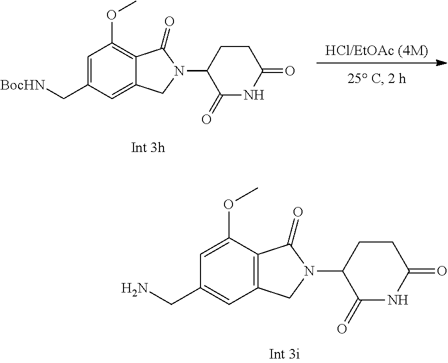 [SMILES: COc1cc(CNC(=O)OC(C)(C)C)cc2c1C(=O)N(C1CCC(=O)NC1=O)C2 ; COc1cc(CN)cc2c1C(=O)N(C1CCC(=O)NC1=O)C2]

To a solution of 3-(5-(aminomethyl)-7-methoxy-1-oxoisoindolin-2-yl)piperidine-2,6-dione, Int 3h, (600 mg, 1.29 mmol, 87% purity, 1.00 eq) in ethyl acetate (5.00 mL) was added hydrochloric acid /ethyl acetate (4.00 M, 323 uL, 1.00 eq). The mixture was stirred at 25° C. for 2 h. The mixture was concentrated under reduced pressure to give a residue. The residue was purified by triturated with ethyl acetate (10.0 mL) and lyophilized to afford 3-(5-(aminomethyl)-7-methoxy-1-oxoisoindolin-2-yl)piperidine-2,6-dione, Int 3i, (370 mg, 1.22 mmol, 94% yield) as a yellow solid. ¹H NMR (400 MHz, DMSO-d₆) δ=10.93 (s, 1H), 8.48 (br s, 2H), 7.27 (s, 1H), 7.19 (s, 1H), 5.01 (dd, J=5.2, 13.3 Hz, 1H), 4.38-4.19 (m, 2H), 4.12-4.06 (m, 2H), 3.87 (s, 3H), 2.97-2.80 (m, 1H), 2.56 (br d, J=17.2 Hz, 1H), 2.37-2.29 (m, 1H), 1.97-1.92 (m, 1H). MS (ESI) m/z 304.3 [M+H]⁺

### Step F. Preparation of Int 3j

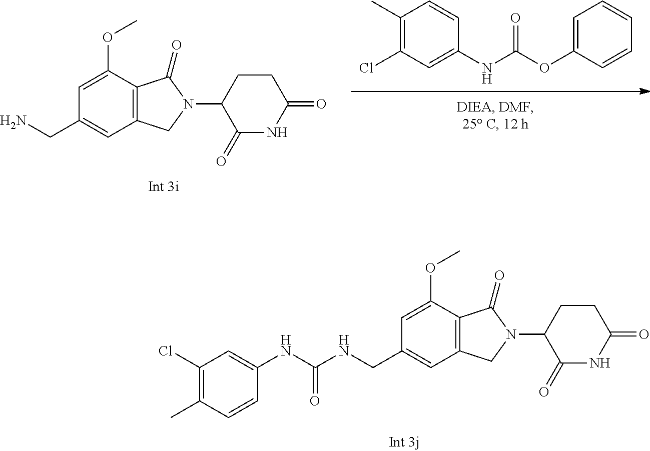 [SMILES: COc1cc(CN)cc2c1C(=O)N(C1CCC(=O)NC1=O)C2 ; Cc1ccc(NC(=O)Oc2ccccc2)cc1Cl ; COc1cc(CNC(=O)Nc2ccc(C)c(Cl)c2)cc2c1C(=O)N(C1CCC(=O)NC1=O)C2]

To a solution of 3-(5-(aminomethyl)-7-methoxy-1-oxoisoindolin-2-yl)piperidine-2,6-dione, Int 3i, (370 mg, 1.22 mmol, 1.00 eq) in N,N-dimethylformamide (4.00 mL) was added phenyl (3-chloro-4-methylphenyl)carbamate (245 mg, 1.46 mmol, 201 uL, 1.20 eq) and diisopropylethylamine (472 mg, 3.66 mmol, 637 uL, 3.00 eq). The mixture was stirred at 25° C. for 12 h. The mixture was concentrated under reduced pressure to give a residue. The residue was purified by reversed-phase HPLC (C18, 80 g; condition: water/acetonitrile=1/0-0/1, 0.1% formic acid) and lyophilized to afford 1-(3-chloro-4-methylphenyl)-3-((2-(2,6-dioxopiperidin-3-yl)-7-methoxy-1-oxoisoindolin-5-yl)methyl)urea, Int 3j, (110 mg, 203 umol, 16% yield, 87% purity) as a white solid. ¹H NMR (400 MHz, DMSO-d₆) δ=10.95 (s, 1H), 8.79-8.72 (m, 1H), 7.66 (d, J=1.6 Hz, 1H), 7.20-7.10 (m, 2H), 7.00 (d, J=18.8 Hz, 2H), 6.82-6.75 (m, 1H), 5.01 (dd, J=5.2, 13.3 Hz, 1H), 4.42-4.29 (m, 3H), 4.25-4.16 (m, 1H), 3.85 (s, 3H), 2.96-2.84 (m, 1H), 2.59-2.54 (m, 1H), 2.35-2.30 (m, 1H), 2.22 (s, 3H), 1.95 (br dd, J=5.2, 10.9 Hz, 1H). MS (ESI) m/z 471.3 [M+H]⁻

### Step G. Preparation of Compound IG-24

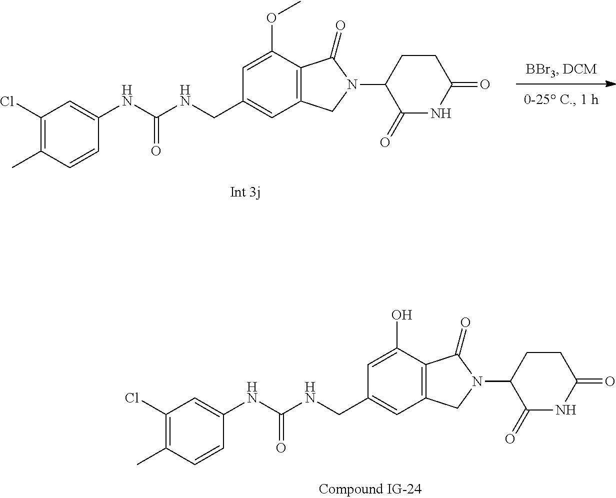 [SMILES: COc1cc(CNC(=O)Nc2ccc(C)c(Cl)c2)cc2c1C(=O)N(C1CCC(=O)NC1=O)C2 ; Cc1ccc(NC(=O)NCc2cc(O)c3c(c2)CN(C2CCC(=O)NC2=O)C3=O)cc1Cl]

To a solution of 1-(3-chloro-4-methylphenyl)-3-((2-(2,6-dioxopiperidin-3-yl)-7-methoxy-1-oxoisoindolin-5-yl)methyl)urea, Int 3j, (50.0 mg, 106 umol, 1.00 eq) in dichloromethane (5.00 mL) was added Boron tribromide (266 mg, 1.06 mmol, 102 uL, 10.0 eq) at 0° C. The mixture was stirred at 25° C. for 1 h. The mixture was quenched with water (5.00 mL) and concentrated under reduced pressure to removed dichloromethane (5.00 mL) to give a residue. The residue was purified by reversed-phase HPLC (C18, 40 g; condition: water/acetonitrile=1/0-0/1, 0.1% formic acid) and lyophilized to afford 1-(3-chloro-4-methylphenyl)-3-((2-(2,6-dioxopiperidin-3-yl)-7-hydroxy-1-oxoisoindolin-5-yl)methyl)urea, Compound IG-24, (5.86 mg, 12.5 umol, 12% yield, 98% purity) as a white solid. ¹H NMR (400 MHz, DMSO-d₆) δ=10.95 (s, 1H), 9.82-9.67 (m, IH), 8.79 (s, 1H), 7.66 (d, J=2.0 Hz, 1H), 7.19-7.11 (m, 2H), 6.88 (br s, 1H), 6.84-6.71 (m, 2H), 5.01 (dd, J=5.2, 13.0 Hz, 1H), 4.36-4.17 (m, 4H), 2.96-2.83 (m, 1H), 2.60 (br s, 1H), 2.44-2.34 (m, 1H), 2.23 (s, 3H), 1.99-1.92 (m, 1H). MS (ESI) m/z 457.3 [M+H]⁺

### Example 14

### Synthesis of 5-amino-2-(2,6-dioxopiperidin-3-yl)-1,3-dioxoisoindolin-4-yl 2-(trifluoromethoxy)benzenesulfonate (Compound IG-29) and 7-amino-2-(2,6-dioxopiperidin-3-yl)-1,3-dioxoisoindolin-4-yl 2-(trifluoromethoxy)benzenesulfonate (Compound IG-30)

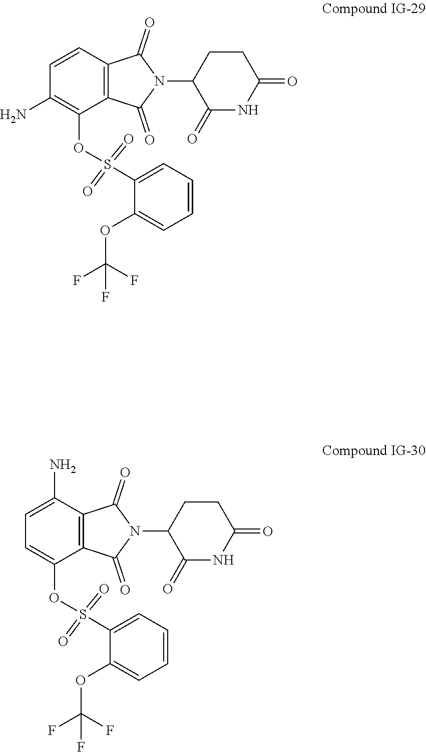 [SMILES: Nc1ccc2c(c1OS(=O)(=O)c1ccccc1OC(F)(F)F)C(=O)N(C1CCC(=O)NC1=O)C2=O ; Nc1ccc(OS(=O)(=O)c2ccccc2OC(F)(F)F)c2c1C(=O)N(C1CCC(=O)NC1=O)C2=O]

### Step A. Preparation of Int 3k and 3l

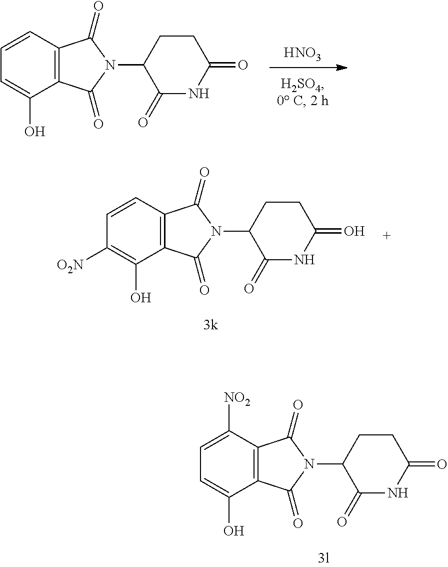 [SMILES: O=C1CCC(N2C(=O)c3cccc(O)c3C2=O)C(=O)N1 ; "HNO3" ; O=C1CCC(N2C(=O)c3ccc([N+](=O)[O-])c(O)c3C2=O)C(=O)N1 ; O=C1CCC(N2C(=O)c3c(O)ccc([N+](=O)[O-])c3C2=O)C(=O)N1]

To a solution of 2-(2,6-dioxopiperidin-3-yl)-4-hydroxyisoindoline-1,3-dione (1.00 g, 3.65 mmol, 1.00 eq) in sulfuric acid (10.0 mL) was added a mixture of sulfuric acid (10.0 mL) and nitric acid (389 mg, 4.01 mmol, 278 uL, 65% purity, 1.10 eq). The mixture was stirred at 0° C. for 2 h. The reaction are added to the ice water (1000 mL) and filtered to give residue. The residue was purified by flash silica gel chromatography (ISCO®; 20 g SepaFlash® Silica Flash Column, Eluent of 0~80% ethyl acetate/petroleum ether gradient @100 mL/min) to give the mixture of 2-(2,6-dioxopiperidin-3-yl)-4-hydroxy-5-nitroisoindoline-1,3-dione, Int 3k, and 2-(2,6-dioxopiperidin-3-yl)-4-hydroxy-7-nitroisoindoline-1,3-dione, Int 31, (1.20 g, crude) as a yellow solid. MS (ESI) m/z 320.0 [M+H]⁺

### Step B. Preparation of Int 3m and 3n

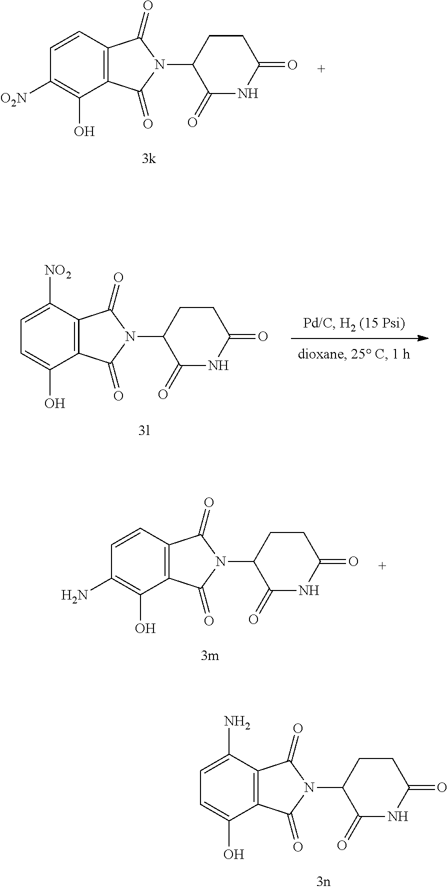 [SMILES: O=C1CCC(N2C(=O)c3ccc([N+](=O)[O-])c(O)c3C2=O)C(=O)N1 ; O=C1CCC(N2C(=O)c3c(O)ccc([N+](=O)[O-])c3C2=O)C(=O)N1 ; "Pd/C, H2 (15 Psi)" ; Nc1ccc2c(c1O)C(=O)N(C1CCC(=O)NC1=O)C2=O ; Nc1ccc(O)c2c1C(=O)N(C1CCC(=O)NC1=O)C2=O]

To a solution of the mixture of 2-(2,6-dioxopiperidin-3-yl)-4-hydroxy-5-nitroisoindoline-1,3-dione, Int 3k, and 2-(2,6-dioxopiperidin-3-yl)-4-hydroxy-7-nitroisoindoline-1,3-dione, Int 31, (1.10 g, 3.45 mmol, 1.00 eq) in dioxane (20.0 mL) was added palladium on carbon (10%, 10 mg) under nitrogen atmosphere. The suspension was degassed and purged with hydrogen for 3 times. The mixture was stirred under hydrogen (15 Psi) at 25° C. for 1 h. The reaction mixture was filtered to give filtrate. The filtrate was concentrated to give a mixture of 5-amino-2-(2,6-dioxopiperidin-3-yl)-4-hydroxyisoindoline-1,3-dione, Int 3m, and 4-amino-2-(2,6-dioxopiperidin-3-yl)-7-hydroxyisoindoline-1,3-dione, Int 3n, (1.00 g, crude) as blue oil. (LCMS and ¹H NMR were from pilot run)¹H NMR (400 MHz, DMSO-d₆) δ=10.97 (br s, 1H), 7.05 (br d, J=8.0 Hz, 1H), 6.72 (d, J=7.6 Hz, 1H), 5.98 (br s, 2H), 4.91 (dd, J=5.6, 12.8 Hz, 2H), 2.85-2.73 (m, 2H), 2.52 (br d, J=2.4 Hz, 1H), 1.96-1.87 (m, 2H). MS (ESI) m/z 290.1 [M+H]⁺

### Step C. Preparation of Compound IG-29 and Compound IG-30

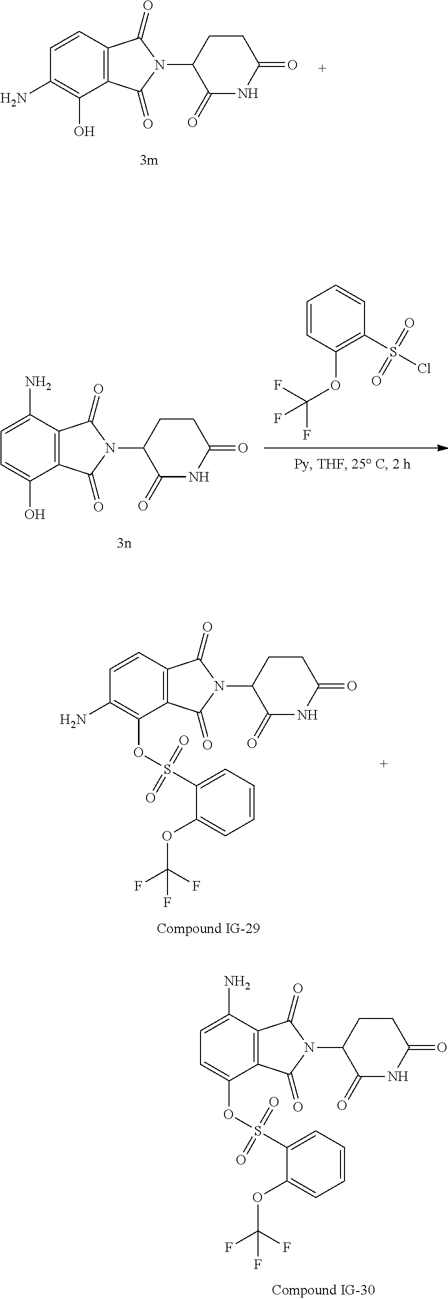

To a solution of the mixture of 5-amino-2-(2,6-dioxopiperidin-3-yl)-4-hydroxyisoindoline-1,3-dione, Int 3m, and -4-amino-2-(2,6-dioxopiperidin-3-yl)-7-hydroxyisoindoline-1,3-dione, Int 3n, (200 mg, 691 mol, 1.00 eq) in tetrahydrofuran (5.00 mL) was added pyridine (140 mg, 1.38 mmol, 192 uL, 2.00 eq) and 2-(trifluoromethoxy)benzenesulfonyl chloride (180 mg, 691 mol, 1.00 eq). The mixture was stirred at 25° C. for 2 h. The reaction mixture was concentrated under reduced pressure to give a residue. The residue was purified by prep-HPLC (column: Phenomenex luna Cis 150*25 mm*10 um; mobile phase: [water (FA)-ACN]; B %: 35%-65%, 10 min) to give 5-amino-2-(2,6-dioxopiperidin-3-yl)-1,3-dioxoisoindolin-4-yl 2-(trifluoromethoxy)benzenesulfonate, Compound IG-29, (69.9 mg, 133 μmol, 19% yield, 98% purity) as a yellow solid and 7-amino-2-(2,6-dioxopiperidin-3-yl)-1,3-dioxoisoindolin-4-yl 2-(trifluoromethoxy)benzenesulfonate, Compound IG-30, (15.1 mg, 28.4 gmol, 4% yield, 97% purity) as a yellow solid.

Compound IG-29 ¹H NMR (400 MHz, DMSO-d₆) δ=11.03 (s, 1H), 8.02 (dd, J=1.2, 7.9 Hz, 1H), 7.97-7.90 (m, 1H), 7.69 (br d, J=8.0 Hz, 1H), 7.62 (t, J=7.7 Hz, 1H), 7.55 (d, J=8.0 Hz, 1H), 7.10 (d, J=8.4 Hz, 1H), 6.30 (s, 2H), 4.90 (dd, J=5.6, 12.9 Hz, 1H), 2.89-2.76 (m, 1H), 2.61-2.53 (m, 1H), 2.36-2.27 (m, 1H), 2.07 (s, 1H), 1.93-1.81 (m, 1H). MS (ESI) m/z 514.0 [M+H]⁺.

Compound IG-30 ¹H NIR (400 MHz, DMSO-d)=11.08 (s, 1H), 7.94 (br dd, J=3.6, 7.8 Hz, 2H), 7.72 (br d, J=8.4 Hz, 1H), 7.65-7.57 (m, 1H), 7.01 (q, J=9.2 Hz, 2H), 6.72 (br s, 2H), 4.96 (br dd, J=5.6, 12.6 Hz, 1H), 2.95-2.78 (m, 1H), 2.58 (br s, 1H), 2.41-2.36 (m, 1H), 1.93 (br dd, J=4.4, 10.4 Hz, 1H). MS (ESI) m/z 514.0 [M+H]⁺.

### Example 15

### Synthesis of 1-(3-chloro-4-methylphenyl)-3-((2-(2,6-dioxopiperidin-3-yl)-4-(methylamino)-1-oxoisoindolin-5-yl)methyl)urea (Compound IG-31)

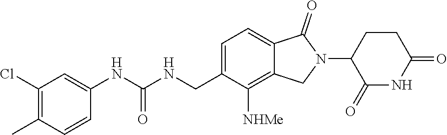 [SMILES: CNc1c(CNC(=O)Nc2ccc(C)c(Cl)c2)ccc2c1CN(C1CCC(=O)NC1=O)C2=O]

### Step A. Preparation of Int 3o

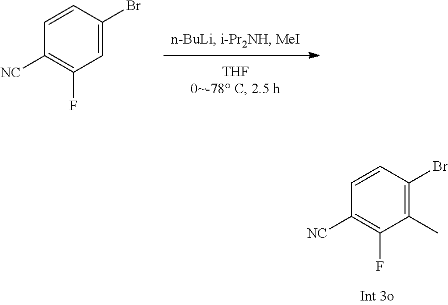

To a solution of diisopropylamine (6.07 g, 60.00 mmol, 8.48 mL, 1.20 eq) in tetrahydrofuran (50.0 mL) was added n-butyllithium (2.5 M, 22.00 mL, 1.10 eq) dropwise at −78° C., the mixture was stirred at −78° C. for 0.5 h. Then a solution of 4 4-bromo-2-fluorobenzonitrile (10.0 g, 50.0 mmol, 1.00 eq) in tetrahydrofuran (50.0 mL) was added at −78° C., then iodomethane (7.81 g, 55.0 mmol, 3.42 mL, 1.10 eq) was added to, the mixture was stirred at −78° C. for 2 h under nitrogen atmosphere. The mixture was quenched with saturated ammonium chloride (100 mL) and extracted with ethyl acetate (3×100 mL). The combined organic layers were washed with brine (20.0 mL), dried over sodium sulfate and filtered. The filtrate was concentrated under reduced pressure to give a residue. The residue was purified by column chromatography (SiO2, petroleum ether/ethyl acetate=10/1) to give 4-bromo-2-fluoro-3-methylbenzonitrile, Int 3o, (3.50 g, 14.7 mmol, 29% yield, 90% purity) as a yellow solid. ¹H NMR (400 MHz, DMSO-d₆) δ=7.40 (d, 1H), 7.25 (m, 1H), 2.31 (t, 3H).

### Step B. Preparation of Int 3p

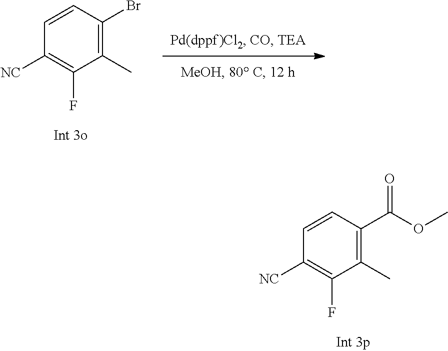

To a solution of 4-bromo-2-fluoro-3-methylbenzonitrile, Int 3o, (1.00 g, 4.67 mmol, 1.00 eq) in methanol (10.0 mL) were added triethylamine (473 mg, 4.67 mmol, 650 uL, 1.00 eq) and [1, 1-bis(diphenylphosphino)ferrocene]dichloropalladium(II) (342 mg, 467 umol, 0.100 eq). The mixture was stirred at 80° C. for 12 h under carbon monoxide atmosphere (50 Psi). The mixture was concentrated under reduced pressure to give a residue. The residue was purified by column chromatography on silica gel (petroleum ether/ethyl acetate=4/1) to give methyl 4-cyano-3-fluoro-2-methylbenzoate, Int 3p, (810 mg, 4.19 mmol, 89% yield) as a white solid. 11H NMR (400 MHz, DMSO-d₆) δ=7.89 (t, J=7.2 Hz, 1H), 7.74 (d, J=8.0 Hz, 1H), 3.88 (s, 3H), 2.42 (s, 3H).

### Step C. Preparation of Int 3q

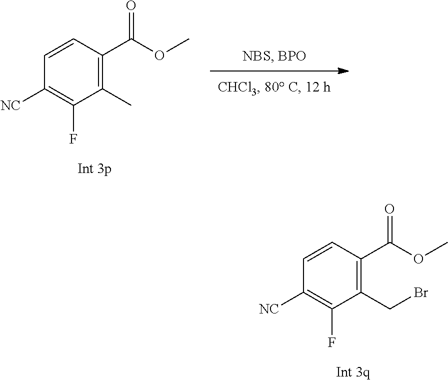

To a solution of methyl 4-cyano-3-fluoro-2-methyl-benzoate, Int 3p, (760 mg, 3.93 mmol, 1.00 eq) in chloroform (15.0 mL) was added N-bromosuccinimide (770 mg, 4.33 mmol, 1.10 eq) and benzoyl peroxide (95.3 mg, 393 umol, 0.100 eq). The mixture was stirred at 80° C. for 12 h under nitrogen atmosphere. The mixture was concentrated under reduced pressure to give a residue. The residue was purified by column chromatography on silica gel (petroleum ether/ethyl acetate=5/1) to give methyl 2-(bromomethyl)-4-cyano-3-fluorobenzoate, Int 3q, (500 mg, 1.84 mmol, 46% yield) as a white solid. ¹H NMR (400 MHz, DMSO-d₆) δ=8.08 (dd, J=6.4, 8.1 Hz, 1H), 7.85 (d, J=8.4 Hz, 1H), 4.92 (d, J=2.0 Hz, 2H), 3.93 (s, 3H).

### Step D. Preparation of Int 3r

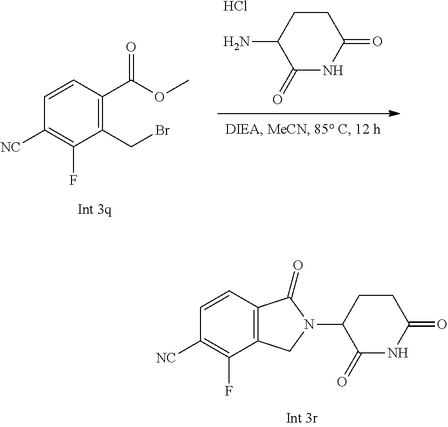 [SMILES: Cl ; COC(=O)c1ccc(C#N)c(F)c1CBr ; NC1CCC(=O)NC1=O ; N#Cc1ccc2c(c1F)CN(C1CCC(=O)NC1=O)C2=O]

To a solution of methyl 2-(bromomethyl)-4-cyano-3-fluorobenzoate, Int 3q, (650 mg, 2.39 mmol, 1.00 eq) in acetonitrile (10.0 mL) was added N,N-diisopropylethylamine (926 mg, 7.17 mmol, 1.25 mL, 3.00 eq) and 3-aminopiperidine-2,6-dione (393 mg, 2.39 mmol, 1.00 eq, hydrogen chloride). The mixture was stirred at 85° C. for 12 h. The mixture was concentrated under reduced pressure to give a residue. The residue was triturated with water (25.0 mL) at 25° C. for 15 min and collected by filtration to give 2-(2,6-dioxopiperidin-3-yl)-4-fluoro-1-oxoisoindoline-5-carbonitrile, Int 3r, (650 mg, 2.26 mmol, 94% yield) as a white solid. ¹H NMR (400 MHz, DMSO-d₆) δ=11.05 (s, 1H), 8.10 (dd, J=5.6, 7.6 Hz, 1H), 7.78 (d, J=8.0 Hz, 1H), 5.16 (dd, J=5.2, 13.2 Hz, 1H), 4.72-4.62 (m, 1H), 4.58-4.43 (m, 1H), 2.99-2.84 (m, 1H), 2.61 (br d, J=17.2 Hz, 1H), 2.44-2.41 (m, 1H), 2.05-1.98 (m, 1H).

### Step E. Preparation of Int 3s

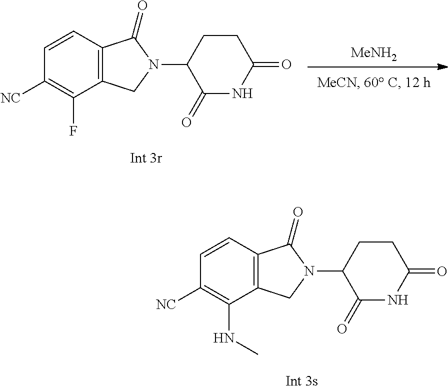 [SMILES: "MeNH2" ; N#Cc1ccc2c(c1F)CN(C1CCC(=O)NC1=O)C2=O ; CNc1c(C#N)ccc2c1CN(C1CCC(=O)NC1=O)C2=O]

To a solution of 2-(2,6-dioxopiperidin-3-yl)-4-fluoro-1-oxoisoindoline-5-carbonitrile, Int 3r, (500 mg, 1.74 mmol, 1.00 eq) in acetonitrile (10.0 mL) was added methylamine (2 M, 8.70 mL, 10 eq). The mixture was stirred at 60° C. for 12 h. Ethyl acetate (40.0 mL) and water (40.0 mL) were added and layers were separated. The aqueous phase was extracted with ethyl acetate (2×30.0 mL). Combined extracts were washed with brine (40.0 mL), dried over sodium sulfate and filtered. The filtrate was concentrated under reduced pressure to give a residue. The residue was purified by prep-HPLC (0.1% FA conditions) to give crude product. The crude product was triturated with ethyl acetate (5.00 mL) at 25° C. for 15 min and collected by filtration to give 2-(2,6-dioxopiperidin-3-yl)-4-(methylamino)-1-oxoisoindoline-5-carbonitrile, Int 3s, (100 mg, 335 μmol, 19% yield) as a white solid. ¹H NMR (400 MHz, DMSO-d₆) δ=11.02 (br s, 1H), 7.56 (d, J=8.0 Hz, 1H), 6.94 (d, J=7.9 Hz, 1H), 6.42 (br d, J=5.2 Hz, 1H), 5.10 (dd, J=5.2, 13.3 Hz, 1H), 4.58-4.37 (m, 2H), 3.15-3.09 (m, 3H), 2.96-2.86 (m, 1H), 2.63-2.58 (m, 1H), 2.39-2.32 (m, 1H), 2.06-1.98 (m, 1H).

### Step F. Preparation of Int 3t

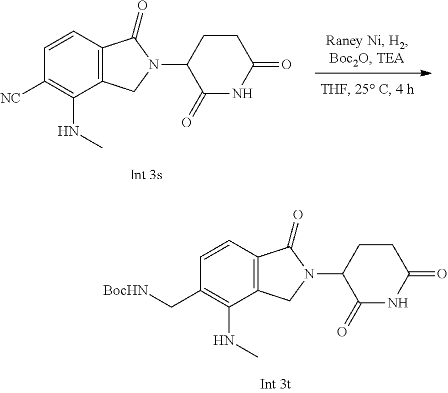 [SMILES: CNc1c(C#N)ccc2c1CN(C1CCC(=O)NC1=O)C2=O ; "Raney Ni, H2,Boc2O, TEA" ; CNc1c(CNC(=O)OC(C)(C)C)ccc2c1CN(C1CCC(=O)NC1=O)C2=O]

To a solution of 2-(2,6-dioxopiperidin-3-yl)-4-(methylamino)-1-oxoisoindoline-5-carbonitrile, Int 3s, (100 mg, 335 μmol, 1.00 eq) in tetrahydrofuran (3.00 mL) was added triethylamine (204 mg, 2.01 mmol, 280 μL, 6.00 eq), di-tert-butyl dicarbonate (219 mg, 1.01 mmol, 231 μL, 3.00 eq) and Raney-Ni (50.0 mg) under nitrogen atmosphere. The mixture was stirred at 25° C. for 4 h under hydrogen atmosphere (15Psi). The resulting mixture was filtered over Celite and the filtrate was concentrated under reduced pressure to give crude product. The crude product was triturated with ethyl acetate (3.00 mL) at 25° C. for 10 minutes and collected by filtration to give tert-butyl ((2-(2,6-dioxopiperidin-3-yl)-4-(methylamino)-1-oxoisoindolin-5-yl)methyl)carbamate, Int 3t, (56 mg, 139 mol, 41% yield) as a white solid. ¹H NMR (400 MHz, DMSO-d₆) δ=10.97 (s, 1H), 7.36 (br t, J=6.0 Hz, 1H), 7.13 (d, J=7.6 Hz, 1H), 6.95 (d, J=7.6 Hz, 1H), 5.22 (br d, J=4.8 Hz, 1H), 5.08 (dd, J=5.2, 13.2 Hz, 1H), 4.72-4.49 (m, 2H), 4.04 (br d, J=6.0 Hz, 2H), 2.97 (d, J=5.2 Hz, 3H), 2.94-2.84 (m, 1H), 2.64-2.53 (m, 1H), 2.46-2.38 (m, 1H), 2.01-1.94 (m, 1H), 1.40 (s, 9H).

### Step G. Preparation of Int 3u

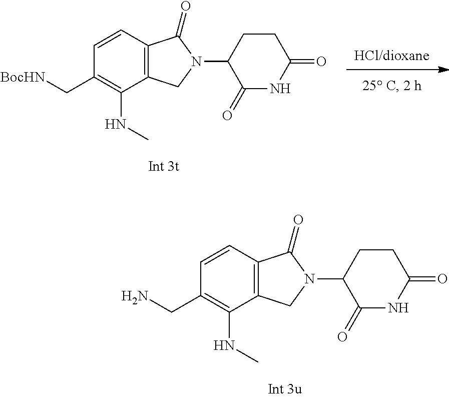 [SMILES: CNc1c(CNC(=O)OC(C)(C)C)ccc2c1CN(C1CCC(=O)NC1=O)C2=O ; "HCl/dioxane" ; CNc1c(CN)ccc2c1CN(C1CCC(=O)NC1=O)C2=O]

A solution of tert-butyl ((2-(2,6-dioxopiperidin-3-yl)-4-(methylamino)-1-oxoisoindolin-5-yl)methyl) carbamate, Int 3t, (56.0 mg, 139 μmol, 1.00 eq) in hydrochloric acid/dioxane (4M, 1.00 mL) was stirred at 25° C. for 2 h. The mixture was concentrated under reduced pressure to give 3-(5-(aminomethyl)-4-(methylamino)-1-oxoisoindolin-2-yl)piperidine-2,6-dione, Int 3u, (45 mg, crude) as a white solid. MS (ESI) m/z 286.2 [M+H]⁺

### Step H. Preparation of Compound IG-31

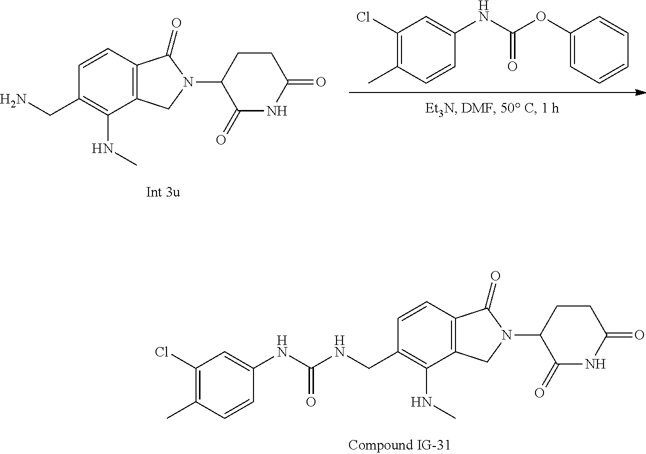 [SMILES: CNc1c(CN)ccc2c1CN(C1CCC(=O)NC1=O)C2=O ; Cc1ccc(NC(=O)Oc2ccccc2)cc1Cl ; CNc1c(CNC(=O)Nc2ccc(C)c(Cl)c2)ccc2c1CN(C1CCC(=O)NC1=O)C2=O]

To a solution of 3-(5-(aminomethyl)-4-(methylamino)-1-oxoisoindolin-2-yl)piperidine-2,6-dione, Int 3u, (45.0 mg, 149 μmol, 1.00 eq) in dimethyl formamide (2.00 mL) was added triethylamine (60.3 mg, 595 μmol, 82.9 μL, 4.00 eq) and phenyl (3-chloro-4-methylphenyl)carbamate (42.9 mg, 164 gmol, 1.10 eq). The mixture was stirred at 50° C. for 1 h. The mixture was concentrated under reduced pressure to give a residue. The residue was purified by reversed-phase HPLC (column: Phenomenex luna C18 150*25 mm*10 um; mobile phase: [water(FA)-ACN]; B %: 25%-55%, 10 min) and lyophilized to afford 1-(3-chloro-4-methylphenyl)-3-((2-(2,6-dioxopiperidin-3-yl)-4-(methylamino)-1-oxoisoindolin-5-yl)methyl)urea, Compound IG-31, (39.83 mg, 83.9 μmol, 56% yield, 99% purity) as a white solid. ¹H NMR (400 MHz, DMSO-d₆) δ=10.95 (br s, 1H), 8.81-8.75 (m, 1H), 7.63 (d, J=2.0 Hz, 1H), 7.22 (d, J=7.6 Hz, 1H), 7.20-7.16 (m, 1H), 7.15-7.10 (m, 1H), 6.96 (d, J=7.6 Hz, 1H), 6.67 (br d, J=5.6 Hz, 1H), 5.44 (q, J=5.2 Hz, 1H), 5.08 (dd, J=5.2, 13.3 Hz, 1H), 4.74-4.50 (m, 2H), 4.23 (d, J=6.0 Hz, 2H), 2.99 (d, J=5.1 Hz, 3H), 2.96-2.86 (m, 1H), 2.63-2.55 (m, 1H), 2.48-2.39 (m, 1H), 2.23 (s, 3H), 2.04-1.92 (m, 1H). MS (ESI) m/z 470.1 [M+H]⁺

### Example 16

### Synthesis of (2-(2,6-dioxopiperidin-3-yl)-4-hydroxy-1-oxoisoindolin-5-yl)methyl (3-chloro-4-methylphenyl)carbamate (Compound IG-33)

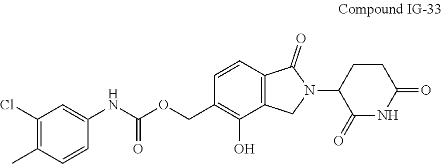 [SMILES: Cc1ccc(NC(=O)OCc2ccc3c(c2O)CN(C2CCC(=O)NC2=O)C3=O)cc1Cl]

### Step A. Preparation of Int 3v

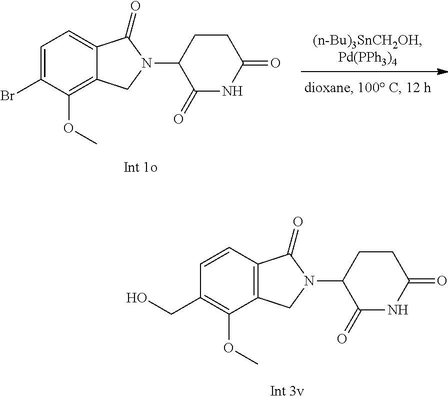

To a solution of 3-(5-bromo-4-methoxy-1-oxoisoindolin-2-yl)piperidine-2,6-dione, Int 1o, (600 mg, 1.70 mmol, 1.00 eq) in dioxane (20.0 mL) was added tetrakis[triphenylphosphine]palladium(0) (255 mg, 220 gmol, 0.130 eq) and tributylstannylmethanol (2.73 g, 8.49 mmol, 5.00 eq). The mixture was stirred at 100° C. for 12 h under nitrogen atmosphere. The reaction mixture was concentrated under reduced pressure to give a residue. The residue was purified by column chromatography on silica gel (petroleum ether/ethyl acetate=0/1) to give 3-(5-(hydroxymethyl)-4-methoxy-1-oxoisoindolin-2-yl)piperidine-2,6-dione, Int 3v, (250 mg, 821 gmol, 48% yield) as a green solid. ¹H NMR (400 MHz, DMSO-d₆) δ=10.99 (s, 1H), 7.56 (d, J=7.6 Hz, 1H), 7.42 (d, J=7.6 Hz, 1H), 5.24 (t, J=5.6 Hz, 1H), 5.10 (dd, J=5.1, 13.2 Hz, 1H), 4.71-4.64 (m, 1H), 4.58 (d, J=5.4 Hz, 2H), 4.53-4.46 (m, 1H), 3.92 (s, 3H), 3.00-2.86 (m, 1H), 2.60 (br d, J=17.8 Hz, 1H), 2.47-2.38 (m, 1H), 2.05-1.99 (m, 1H).

### Step B. Preparation of Int 3w

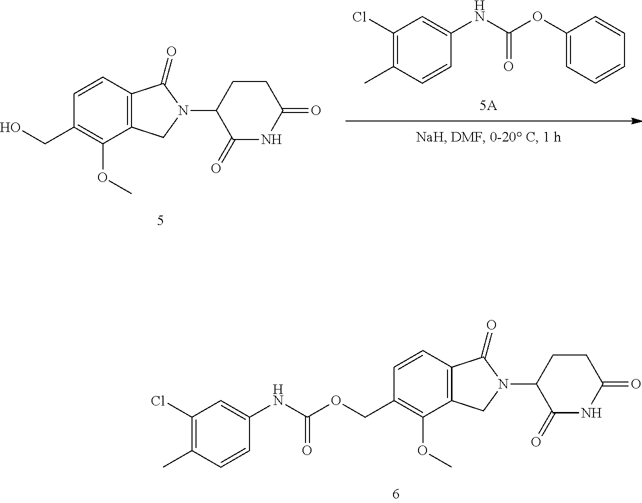 [SMILES: COc1c(CO)ccc2c1CN(C1CCC(=O)NC1=O)C2=O ; Cc1ccc(NC(=O)Oc2ccccc2)cc1Cl ; COc1c(COC(=O)Nc2ccc(C)c(Cl)c2)ccc2c1CN(C1CCC(=O)NC1=O)C2=O]

To a solution of 3-(5-(hydroxymethyl)-4-methoxy-1-oxoisoindolin-2-yl)piperidine-2,6-dione, Int 3v, (250 mg, 821 gmol, 1.00 eq) in dimethylformamide (6.00 mL) was added phenyl (3-chloro-4-methylphenyl)carbamate (236 mg, 903 gmol, 1.10 eq) and sodium hydride (65.7 mg, 1.64 mmol, 60% purity, 2.00 eq). The mixture was stirred at 0-25° C. for 1 h. The reaction mixture was quenched by addition water (20.0 mL) at 25° C., collect the crystalline solid by suction filtration, washed with 2 mL portions of cold water. The solid was dry in vacuo to give crude product. The crude product was triturated with ethyl acetate (10.0 ml) at 25° C. for 5 min and collected by filtration to give (2-(2,6-dioxopiperidin-3-yl)-4-methoxy-1-oxoisoindolin-5-yl)methyl (3-chloro-4-methylphenyl)carbamate, Int 3w, (140 mg, 272 gmol, 33% yield, 92% purity) as a white solid ¹H NMR (400 MHz, DMSO-d₆) δ=11.00 (s, 1H), 9.90 (s, 1H), 7.59 (d, J=1.2 Hz, 1H), 755 (d, J=7.2 Hz, 1H), 7.45 (d, J=7.6 Hz, 1H), 7.30-7.23 (m, 2H), 5.23 (s, 2H), 5.12 (dd, J=4.6, 13.2 Hz, 1H), 4.75 (d, J=17.2 Hz, 1H), 4.57 (d, J=17.2 Hz, 1H), 4.00 (s, 3H), 2.94-2.87 (m, 1H), 2.58 (br d, J=1.2 Hz, 1H), 2.45-2.43 (m, 1H), 2.25 (s, 3H), 2.03-1.98 (m, 1H).

### Step C. Preparation of Compound IG-33

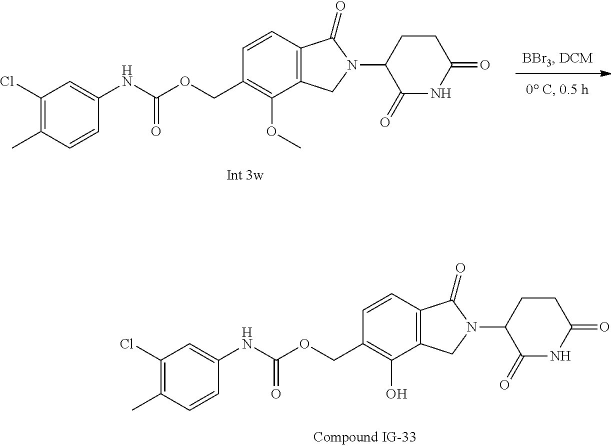 [SMILES: COc1c(COC(=O)Nc2ccc(C)c(Cl)c2)ccc2c1CN(C1CCC(=O)NC1=O)C2=O ; Cc1ccc(NC(=O)OCc2ccc3c(c2O)CN(C2CCC(=O)NC2=O)C3=O)cc1Cl]

To a solution of (2-(2,6-dioxopiperidin-3-yl)-4-methoxy-1-oxoisoindolin-5-yl)methyl (3-chloro-4-methylphenyl)carbamate, Int 3w, (140 mg, 296 μmol, 1.00 eq) in dichloromethane (5.00 mL) was added boron tribromide (743 mg, 2.97 mmol, 286 μL, 10.0 eq). The mixture was stirred at 0° C. for 0.5 h. The reaction mixture was quenched by addition of ice water (20.0 ml) and then concentrated under reduced pressure to give a residue. The residue was purified by column chromatography on silica gel (petroleum ether/ethyl acetate=0/1) to give crude product. The crude product was triturated with ethyl acetate (10.0 ml) at 25° C. for 5 min and collected by filtration to afford (2-(2,6-dioxopiperidin-3-yl)-4-hydroxy-1-oxoisoindolin-5-yl)methyl (3-chloro-4-methylphenyl)carbamate, Compound IG-33, (41.32 mg, 88.4 μmol, 29% yield, 98% purity) as a white solid. ¹H NMR (400 MHz, DMSO-d₆) δ=11.00 (s, 1H), 10.00 (br s, 1H), 9.88 (br s, 1H), 7.60 (s, 1H), 7.49 (d, J=7.6 Hz, 1H), 7.32-7.27 (m, 1H), 7.27-7.24 (m, 2H), 5.24 (s, 2H), 5.11 (dd, J=5.2, 13.2 Hz, 1H), 4.42-4.36 (m, 1H), 4.30-4.24 (m, 1H), 2.96-2.87 (m, 1H), 2.64-2.59 (m, 1H), 2.39-2.33 (m, 1H), 2.26 (s, 3H), 2.07-2.01 (m, 1H). MS (ESI) m/z 458.1 [M+H]⁺

### Example 17 Synthesis of N-(2-(2,6-dioxopiperidin-3-yl)-7-hydroxy-1,3-dioxoisoindolin-5-yl)-2-(trifluoromethoxy)benzenesulfonamide (Compound IG-34)

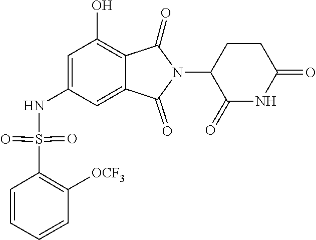 [SMILES: O=C1CCC(N2C(=O)c3cc(NS(=O)(=O)c4ccccc4OC(F)(F)F)cc(O)c3C2=O)C(=O)N1]

### Step A. Preparation of Int 3x

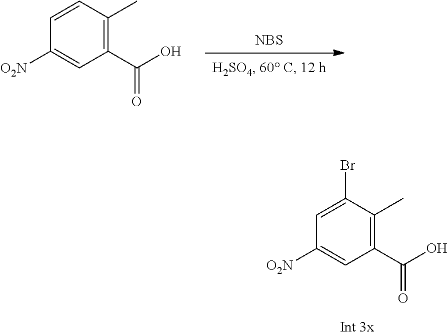

To a solution of 2-methyl-5-nitrobenzoic acid (5.00 g, 27.6 mmol, 1.00 eq) in sulfuric acid (40.0 mL) was slowly added N-bromosuccinimide (5.90 g, 33.1 mmol, 1.20 eq). The mixture was stirred at 60° C. for 12 h. After being cooled to 25° C., the mixture was added into ice-water (100 mL). The mixture was filtered to give filtered cake that was concentrated to give 3-bromo-2-methyl-5-nitrobenzoic acid, Int 3x, (4.70 g, 18.1 mmol, 65% yield) as a yellow solid. ¹H NMR (400 MHz, DMSO-d₆) δ=13.88 (br s, 1H), 8.52 (d, J=1.6 Hz, 1H), 8.43 (d, J=1.6 Hz, 1H), 2.64 (s, 3H).

### Step B. Preparation of Int 3y

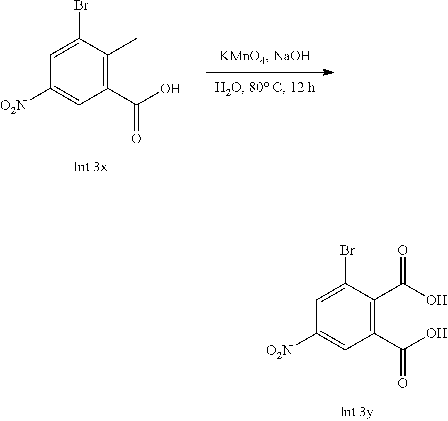

To a solution of 3-bromo-2-methyl-5-nitrobenzoic acid, Int 3x, (4.70 g, 18.1 mmol, 1.00 eq) in water (100 mL) was added sodium hydroxide (2.17 g, 54.2 mmol, 3.00 eq), then was slowly added potassium permanganate (12.2 g, 77.3 mmol, 4.27 eq). The mixture was stirred at 80° C. for 12 h. The mixture was filtered to give filtrate. The filtrate was adjusted pH=1 with hydrochloric acid and extracted with water/ethyl acetate (100 mL/200 mL). The organic layer was collected and concentrated to give 3-bromo-5-nitrophthalic acid, Int 3y, (3.60 g, 11.8 mmol, 65% yield, 95% purity) as a yellow solid. ¹H NMR (400 MHz, DMSO-d₆) δ=14.71-13.32 (m, 2H), 8.69 (d, J=2.4 Hz, 1H), 8.58 (d, J=2.0 Hz, 1H).

### Step C. Preparation of Int 3z

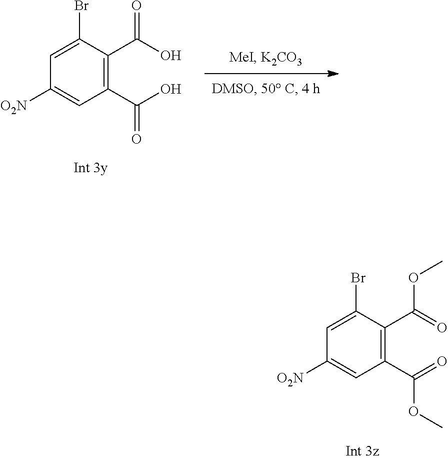

To a solution of 3-bromo-5-nitrophthalic acid, Int 3y, (1.00 g, 3.45 mmol, 1.00 eq) in dimethylsulfoxide (10.0 mL) was added potassium carbonate (953 mg, 6.90 mmol, 2.00 eq) and methyl iodide (4.89 g, 34.5 mmol, 2.15 mL, 10.0 eq). The mixture was stirred at 50° C. for 4 h. The mixture was extracted with water/ethyl acetate (40.0 mL/100 mL). The organic layer was collected and concentrated to give dimethyl 3-bromo-5-nitrophthalate, Int 3z, (1.00 g, 2.67 mmol, 77% yield, 85% purity) as a yellow solid. ¹H NMR (400 MHz, DMSO-d₆) δ=8.80 (d, J=2.0 Hz, 1H), 8.62 (d, J=2.0 Hz, 1H), 3.92 (d, J=1.6 Hz, 6H).

### Step D. Preparation of Int 4a

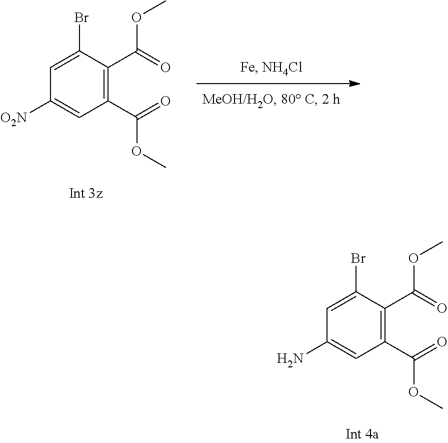 [SMILES: COC(=O)c1cc([N+](=O)[O-])cc(Br)c1C(=O)OC ; COC(=O)c1cc(N)cc(Br)c1C(=O)OC]

To a solution of dimethyl 3-bromo-5-nitrophthalate, Int 3z, (1.00 g, 3.14 mmol, 1.00 eq) in methanol (20.0 mL) and water (5.00 mL) was added iron power (527 mg, 9.43 mmol, 3.00 eq) and ammonium chloride (841 mg, 15.7 mmol, 5.00 eq). The mixture was stirred at 80° C. for 2 h. The mixture was filtered to give a filtrate that was concentrated to give a residue. The residue was extracted with water/ethyl acetate (50.0 mL/100 mL). The organic layer was collected and concentrated to give a residue. The residue was purified by flash silica gel chromatography (ISCO®; 40 g SepaFlash® Silica Flash Column, Fluent of 0~60% Ethyl acetate/Petroleum ether gradient @100 mL/min) to give dimethyl 5-amino-3-bromophthalate, Int 4a, (450 mg, 1.48 mmol, 47% yield, 95% purity) as a yellow solid. ¹H NMR (400 MHz, DMSO-d₆) δ=7.08 (d, J=2.0 Hz, 1H), 6.99 (d, J=2.4 Hz, 1H), 6.03 (s, 2H), 3.78 (s, 3H), 3.74 (s, 3H).

### Step E. Preparation of Int 4b

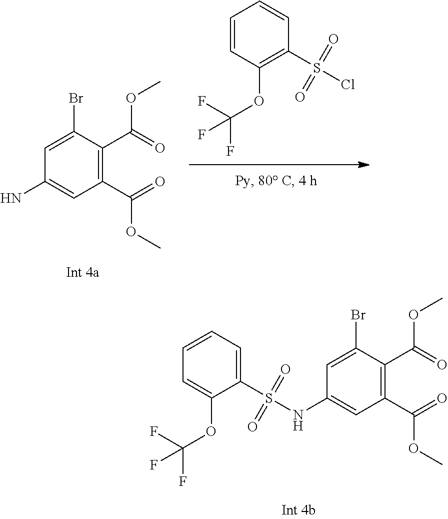

To a solution of dimethyl 5-amino-3-bromophthalate, Int 4a, (410 mg, 1.42 mmol, 1.00 eq) in pyridine (5.00 mL) was added 2-(trifluoromethoxy)benzenesulfonyl chloride (408 mg, 1.57 mmol, 1.10 eq). The mixture was stirred at 80° C. for 4 h. The mixture was concentrated to give a residue. The residue was purified by flash silica gel chromatography (ISCO®; 4 g SepaFlash® Silica Flash Column, Fluent of 40-50% Ethyl acetate/Petroleum ether gradient @80 mL/min) to give dimethyl 3-bromo-5-((2-(trifluoromethoxy)phenyl) sulfonamido)phthalate, Int 4b, (0.400 g, 702 μmol, 49% yield, 90% purity) as a yellow solid. ¹H NMR (400 MHz, DMSO-d₆) δ=11.33 (s, 1H), 8.05 (dd, J=1.4, 8.0 Hz, 1H), 7.82 (dt, J=1.6, 8.0 Hz, 1H), 7.71 (d, J=2.0 Hz, 1H), 7.65-7.57 (m, 3H), 3.81 (s, 3H), 3.80 (s, 3H).

### Step F. Preparation of Int 4c

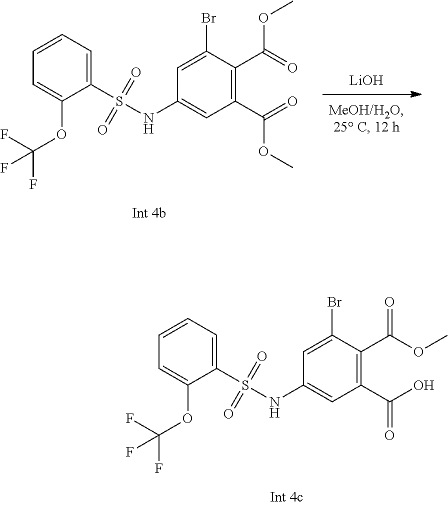 [SMILES: COC(=O)c1cc(NS(=O)(=O)c2ccccc2OC(F)(F)F)cc(Br)c1C(=O)OC ; "LiOH" ; COC(=O)c1c(Br)cc(NS(=O)(=O)c2ccccc2OC(F)(F)F)cc1C(=O)O]

To a solution of dimethyl 3-bromo-5-((2-(trifluoromethoxy)phenyl)sulfonamido)phthalate, Int 4b, (0.400 g, 781 μmol, 1.00 eq) in methanol (1.00 mL) and water (0.300 mL) was added lithium hydroxide (65.5 mg, 1.56 mmol, 2.00 eq). The mixture was stirred at 25° C. for 12 h. The mixture was adjusted pH=3 with hydrochloric acid and concentrated to give a residue. The residue was extracted with water/ethyl acetate (20.0 mL/80.0 mL). The organic layer was collected and concentrated to give 3-bromo-2-(methoxycarbonyl)-5-((2-(trifluoromethoxy) phenyl)sulfonamido)benzoic acid, Int 4c, (360 mg, 722 μmol, 92% yield) as a yellow solid. ¹H NMR (400 MHz, DMSO-d₆) δ=13.5 (s, 1H), 11.28 (s, 1H), 8.04 (dd, J=1.6, 8.0 Hz, 1H), 7.81 (dt, =1.6, 8.0 Hz, 1H), 7.69 (d, J=2.0 Hz, 1H), 7.65-7.53 (m, 3H), 3.76 (s, 3H).

### Step G. Preparation of Int 4d

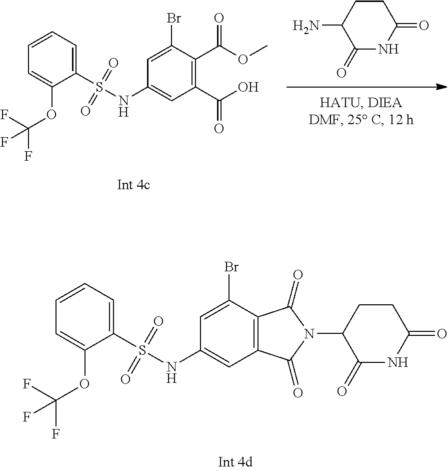 [SMILES: COC(=O)c1c(Br)cc(NS(=O)(=O)c2ccccc2OC(F)(F)F)cc1C(=O)O ; NC1CCC(=O)NC1=O ; O=C1CCC(N2C(=O)c3cc(NS(=O)(=O)c4ccccc4OC(F)(F)F)cc(Br)c3C2=O)C(=O)N1]

To a solution of 3-bromo-2-(methoxycarbonyl)-5-((2-(trifluoromethoxy)phenyl)sulfonamido)benzoic acid, Int 4c, (0.250 g, 502 gmol, 1.00 eq) in dimethyl formamide (10.0 mL) was added O-(7-azabenzotriazol-1-yl)-N,N,N′,N′-tetramethyluronium hexafluorophosphate (382 mg, 1.00 mmol, 2.00 eq), diisopropylethylamine (64.8 mg, 502 μmol, 87.4 μL, 1.00 eq) and 3-aminopiperidine-2,6-dione;hydrochloride (99.1 mg, 602 μmol, 1.20 eq). The mixture was stirred at 25° C. for 12 h. The mixture was filtered to give filtrate. The filtrate was purified by prep-HPLC (0.1% FA). The desired fraction was concentrated and extracted with water/ethyl acetate (20.0 mL/50.0 mL). The organic layer was collected and concentrated to give N-(7-bromo-2-(2,6-dioxopiperidin-3-yl)-1,3-dioxoisoindolin-5-yl)-2-(trifluoromethoxy)benzenesulfonamide, Int 4d, (230 mg, 379 μmol, 75% yield, 95% purity) as a yellow solid. ¹H NMR (400 MHz, DMSO-d₆) δ=11.93 (s, 1H), 11.11 (s, 1H), 8.15-8.09 (m, 1H), 7.84 (dt, J=1.6, 8.0 Hz, 1H), 7.68-7.58 (m, 3H), 7.49 (d, J=1.6 Hz, 1H), 5.09 (dd, J=5.6, 12.8 Hz, 1H), 2.89-2.80 (m, 1H), 2.59 (br d, J=2.4 Hz, 1H), 2.49-2.42 (m, 1H), 2.02 (dt, J=2.4, 5.2 Hz, 1H). MS (ESI) m/z 576.0 [M+H]⁺

### Step H. Preparation of Int 4e

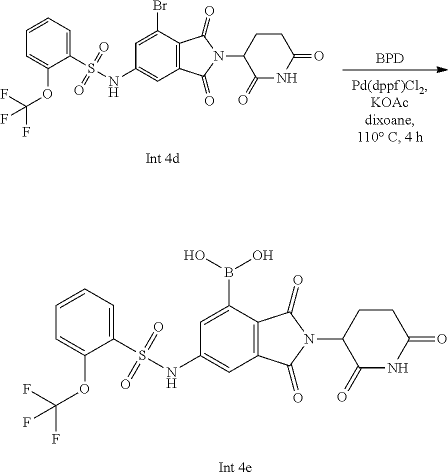

To a solution of N-(7-bromo-2-(2,6-dioxopiperidin-3-yl)-1,3-dioxoisoindolin-5-yl)-2-(trifluoromethoxy) benzenesulfonamide, Int 4d, (200 mg, 347 μmol, 1.00 eq) in dioxane (10.0 mL) was added bis(pinacolato)diboron (132 mg, 521 μmol, 1.50 eq), potassium acetate (68.1 mg, 694 μmol, 2.00 eq) and [1,1′-bis(diphenylphosphino) ferrocene]dichloropalladium(II) (25.4 mg, 34.7 μmol, 0.100 eq) under nitrogen. The mixture was stirred at 110° C. for 4 h under nitrogen. The mixture was concentrated to give a residue. The residue was purified by prep-HPLC (0.1% FA). The desired fraction was collected and concentrated, extracted with water/ethyl acetate (20.0 mL/40.0 mL). The organic layer was collected and concentrated to give (2-(2,6-dioxopiperidin-3-yl)-1,3-dioxo-6-((2-(trifluoromethoxy)phenyl)sulfonamido)isoindolin-4-yl)boronic acid, Int 4e, (100 mg, 166 μmol, 48% yield, 90% purity) as a yellow solid. ¹H NMR (400 MHz, DMSO-d₆) δ=11.54 (s, 1H), 11.12 (s, 1H), 8.75 (br d, J=2.4 Hz, 1H), 8.21-7.99 (m, 2H), 7.86-7.75 (m, 2H), 7.63-7.56 (m, 3H), 5.12 (dd, J=5.6, 12.8 Hz, 1H), 2.91-2.82 (m, 1H), 2.61-2.55 (m, 1H), 2.48-2.41 (m, 1H), 2.08-1.99 (m, 1H). MS (ESI) m/z 542.3 [M+H]⁺

### Step I. Preparation of Compound IG-34

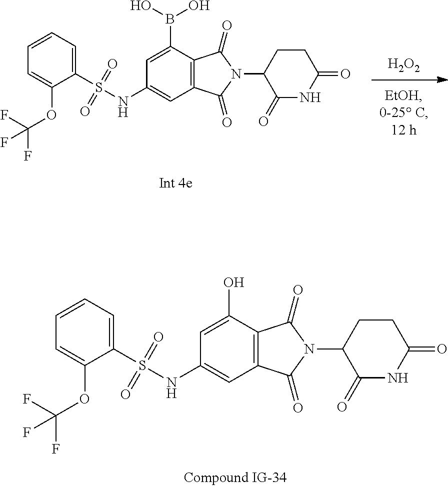 [SMILES: O=C1CCC(N2C(=O)c3cc(NS(=O)(=O)c4ccccc4OC(F)(F)F)cc(B(O)O)c3C2=O)C(=O)N1 ; "H2O2" ; O=C1CCC(N2C(=O)c3cc(NS(=O)(=O)c4ccccc4OC(F)(F)F)cc(O)c3C2=O)C(=O)N1]

To a solution of (2-(2,6-dioxopiperidin-3-yl)-1,3-dioxo-6-((2-(trifluoromethoxy)phenyl)sulfonamido) isoindolin-4-yl)boronic acid, Int 4e, (80.0 mg, 148 μmol, 1.00 eq) in ethanol (1.00 mL) was added hydrogen peroxide (1.74 g, 15.5 mmol, 1.47 mL, 30% purity, 104 eq) at 0° C. The mixture was stirred at 0° C. for 0.5 h. Then the mixture was stirred at 25° C. for 11.5 h. The mixture was concentrated and extracted with water/ethyl acetate (10.0 mL/20.0 mL). The organic layer was collected and concentrated to give a residue. The residue was purified by prep-HPLC (column: Phenomenex luna C18 150*25 mm*10 um; mobile phase: [water (FA)-ACN]; B %: 32%-62%, 7 min). The desired fraction was collected and lyophilized to give N-(2-(2,6-dioxopiperidin-3-yl)-7-hydroxy-1,3-dioxoisoindolin-5-yl)-2-(trifluoromethoxy)benzenesulfonamide, Compound IG-34, (29.05 mg, 54.3 μmol, 37% yield, 96% purity) as an off-white solid. ¹H NMR (400 MHz, DMSO-d₆) δ=11.44 (br s, 1H), 11.30 (br s, 1H), 11.05 (s, 1H), 8.05 (dd, J=1.4, 7.9 Hz, 1H), 7.90-7.74 (m, 1H), 7.66-7.55 (m, 2H), 6.99 (br d, J=4.4 Hz, 2H), 4.99 (dd, J=5.6, 12.8 Hz, 1H), 2.84 (ddd, J=4.8, 13.6, 17.6 Hz, 1H), 2.57 (br d, J=2.4 Hz, 1H), 2.43 (br d, J=4.0 Hz, 1H), 2.02-1.90 (m, 1H). MS (ESI) m/z 514.1 [M+H]⁺

### Example 17

### Synthesis of 1-((3-(2,6-dioxopiperidin-3-yl)-4-oxo-3,4-dihydrobenzo[d][1,2,3]triazin-6-yl)methyl)-3-(3-hydroxy-4-methylphenyl)urea (Compound IG-35)

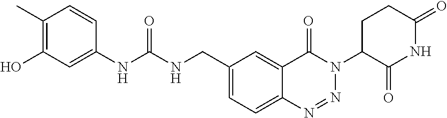 [SMILES: Cc1ccc(NC(=O)NCc2ccc3nnn(C4CCC(=O)NC4=O)c(=O)c3c2)cc1O]

### Step A. Preparation of Int 4f

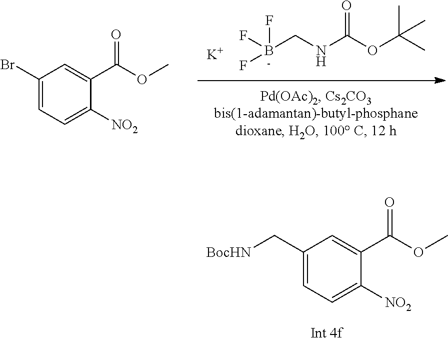

To a solution of methyl 5-bromo-2-nitrobenzoate (0.500 g, 1.92 mmol, 1.00 eq) in the mixture of dioxane (20.0 mL) and water (2.00 mL) was added potassium(I);(((tert-butoxycarbonyl)amino)methyl)trifluoroborate (456 mg, 1.92 mmol, 1.00 eq), cesium carbonate (1.25 g, 3.85 mmol, 2.00 eq), bis(1-adamantyl)-butyl-phosphane (276 mg, 769 umol, 0.400 eq) and palladium(II) acetate (86.3 mg, 385 umol, 0.200 eq). The mixture was stirred at 100° C. for 12 h under nitrogen. The reaction mixture was concentrated under reduced pressure to give a residue. The residue was purified by column chromatography (SiO₂, Petroleum ether/Ethyl acetate=1/0 to 3/1) to give methyl 5-(((tert-butoxycarbonyl)amino)methyl)-2-nitrobenzoate, Int 4f, (1.60 g, 5.16 mmol, 67% yield) as a yellow solid. ¹H NMR (400 MHz, DMSO-d₆) δ=8.05 (d, J=8.4 Hz, 1H), 7.68 (s, 1H), 7.64 (br d, J=8.4 Hz, 1H), 7.61-7.56 (m, 1H), 4.25 (br d, J=6.0 Hz, 2H), 3.85 (s, 3H), 1.39 (s, 9H).

### Step B. Preparation of Int 4g

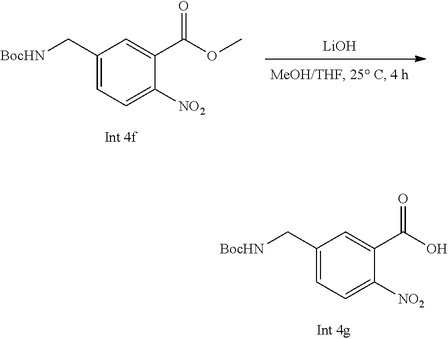 [SMILES: COC(=O)c1cc(CNC(=O)OC(C)(C)C)ccc1[N+](=O)[O-] ; "LiOH" ; CC(C)(C)OC(=O)NCc1ccc([N+](=O)[O-])c(C(=O)O)c1]

To a solution of methyl 5-[(tert-butoxycarbonylamino)methyl]-2-nitro-benzoate, Int 4f, (1.50 g, 4.83 mmol, 1.00 eq) in methanol (7.50 mL) was added a solution of lithium hydroxide (174 mg, 7.25 mmol, 1.50 eq) in tetrahydrofuran (30.0 mL) and water (7.50 mL). The mixture was stirred at 25° C. for 4 h. The reaction mixture was concentrated under reduced pressure to give a residue. The reaction mixture was concentrated and diluted with water. The aqueous solution was acidified with hydrochloric acid (1.00 M, 10.0 ml) to pH=6 and extracted with ethyl acetate (50 mL). The organic layer was dried over anhydrous sodium sulfate and concentrated under reduced pressure to give 5-(((tert-butoxycarbonyl)amino)methyl)-2-nitrobenzoic acid, Int 4g, (1.40 g, 4.73 mmol, 97% yield) as a yellow oil. ¹H NMR (400 MHz, DMSO-d₆) δ=7.96 (d, J=8.4 Hz, 1H), 7.66 (s, 1H), 7.62-7.53 (m, 2H), 4.24 (br d, J=6.0 Hz, 2H), 1.39 (s, 9H). MS (ESI) m/z 197.1 [M+H-100]⁺

### Step C. Preparation of Int 4h

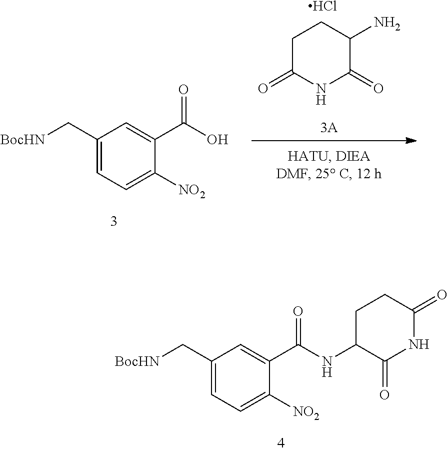

To a solution of 5-(((tert-butoxycarbonyl)amino)methyl)-2-nitrobenzoic acid, Int 4g, (0.700 g, 2.36 mmol, 1.00 eq) in dimethyl formamide (30.0 mL) was added N,N-diisopropylethylamine (1.22 g, 9.45 mmol, 1.65 mL, 4.00 eq), O-(7-Azabenzotriazol-1-yl)-N,N,N′,N′-tetramethyluronium Hexafluorophosphate (1.35 g, 3.54 mmol, 1.50 eq) and 3-aminopiperidine-2,6-dione (467 mg, 2.84 mmol, 1.20 eq, hydrochloric acid). The mixture was stirred at 25° C. for 12 h. The reaction mixture was concentrated under reduced pressure to give a residue. The residue was diluted with water (300 mL) and extracted with ethyl acetate (2×300 mL). The organic phase was dried over sodium sulfate, filtered and concentrated under reduced pressure to give a residue. The residue was purified by column chromatography (SiO₂, Petroleum ether/Ethyl acetate=5/1 to 0/1) to give tert-butyl (3-((2,6-dioxopiperidin-3-yl)carbamoyl)-4-nitrobenzyl)carbamate, Int 4h, (2.56 g, crude) as green oil. ¹H NMR (400 MHz, DMSO-d₆) δ=10.87 (s, 1H), 9.02 (br d, J=8.4 Hz, 1H), 8.06 (d, J=8.4 Hz, 1H), 7.60 (br t, J=6.0 Hz, 1H), 7.53 (dd, J=2.0, 8.4 Hz, 1H), 7.45 (s, 1H), 4.73 (ddd, J=6.0, 8.0, 11.6 Hz, 1H), 4.25 (br d, J=6.0 Hz, 2H), 3.62 (qd, J=6.4, 10.4 Hz, 1H), 3.20-3.08 (m, 1H), 2.84-2.75 (m, 1H), 2.57 (br t, J=3.6 Hz, 1H), 1.40 (s, 9H). MS (ESI) m/z 351.1 [M+H-56]⁺

### Step D. Preparation of Int 4i

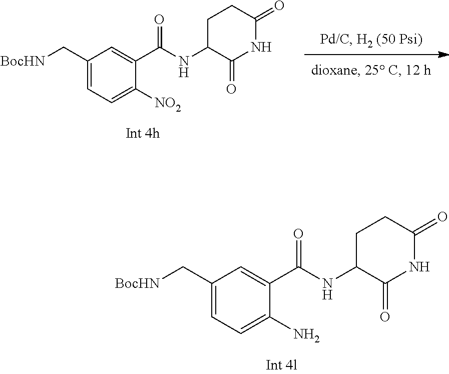 [SMILES: CC(C)(C)OC(=O)NCc1ccc([N+](=O)[O-])c(C(=O)NC2CCC(=O)NC2=O)c1 ; "Pd/C, H2 (50 Psi)" ; CC(C)(C)OC(=O)NCc1ccc(N)c(C(=O)NC2CCC(=O)NC2=O)c1]

To a solution of tert-butyl (3-((2,6-dioxopiperidin-3-yl)carbamoyl)-4-nitrobenzyl)carbamate, Int 4h, (2.20 g, 5.41 mmol, 1.00 eq) in dioxane (30.0 mL) was added palladium on carbon (200 mg, 10% purity). The mixture was stirred at 25° C. for 12 h under hydrogen (50 Psi). The mixture was filtered to give a filter liquor, then was concentrated under reduced pressure to give tert-butyl (4-amino-3-((2,6-dioxopiperidin-3-yl)carbamoyl) benzyl)carbamate, Int 4i, (1.00 g, crude) as a yellow solid. MS (ESI) m/z 321.1 [M+H-56]⁺

### Step E. Preparation of Int 4j

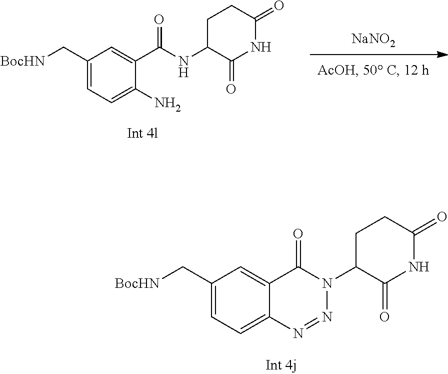 [SMILES: CC(C)(C)OC(=O)NCc1ccc(N)c(C(=O)NC2CCC(=O)NC2=O)c1 ; "NaNO2" ; CC(C)(C)OC(=O)NCc1ccc2nnn(C3CCC(=O)NC3=O)c(=O)c2c1]

To a solution of tert-butyl (4-amino-3-((2,6-dioxopiperidin-3-yl)carbamoyl)benzyl)carbamate, Int 4i, (350 mg, 930 μmol, 1.00 eq) in acetic acid (10.0 mL) was added sodium nitrite (128 mg, 1.86 mmol, 2.00 eq). The mixture was stirred at 50° C. 12 h. The mixture was filtered to give a filter cake, then was concentrated under reduced pressure to give tert-butyl ((3-(2,6-dioxopiperidin-3-yl)-4-oxo-3,4-dihydrobenzo[d][1,2,3]triazin-6-yl)methyl)carbamate, Int 4j, (290 mg, 749 μmol, 80% yield) as a yellow solid, ¹H NMR (400 MHz, DMSO-d₆) δ=11.20 (br s, 1H), 8.22 (d, J=8.4 Hz, 1H), 8.11 (s, 1H), 7.98 (dd, J=1.6, 8.4 Hz, 1H), 7.67 (br t, J=6.0 Hz, 1H), 5.99 (br dd, J=5.6, 12.4 Hz, 1H), 4.37 (br d, J=6.0 Hz, 2H), 2.98-2.92 (m, 1H), 2.71-2.66 (m, 2H), 2.31-2.25 (m, 1H), 1.40 (s, 9H). MS (ESI) m/z 388.3 [M+H]⁺

### Step F. Preparation of Int 4k

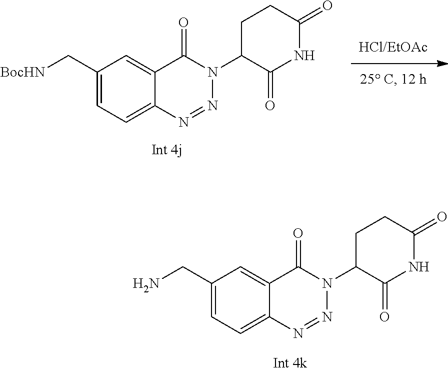 [SMILES: CC(C)(C)OC(=O)NCc1ccc2nnn(C3CCC(=O)NC3=O)c(=O)c2c1 ; NCc1ccc2nnn(C3CCC(=O)NC3=O)c(=O)c2c1]

A solution of tert-butyl ((3-(2,6-dioxopiperidin-3-yl)-4-oxo-3,4-dihydrobenzo[d][1,2,3]triazin-6-yl)methyl) carbamate, Int 4j, (290 mg, 749 μmol, 1.00 eq) in hydrochloric acid/ethyl acetate (4.00 M, 10 mL, 53.4 eq) was stirred at 25° C. for 12 h. The reaction mixture was concentrated under reduced pressure to give 3-(6-(aminomethyl)-4-oxobenzo[d][1,2,3]triazin-3(4H)-yl)piperidine-2,6-dione, Int 4k, (250 mg, crude) as a white solid. ¹H NMR (400 MHz, DMSO-d₆) δ=11.22-11.17 (m, 1H), 8.44 (d, J=1.6 Hz, 1H), 8.34-8.30 (m, 1H), 8.26 (s, 1H), 6.05-5.98 (m, 1H), 4.31 (s, 2H), 3.01-2.94 (m, 1H), 2.76 (br s, 2H), 2.30-2.24 (m, 1H).

### Step G. Preparation of Compound IG-35

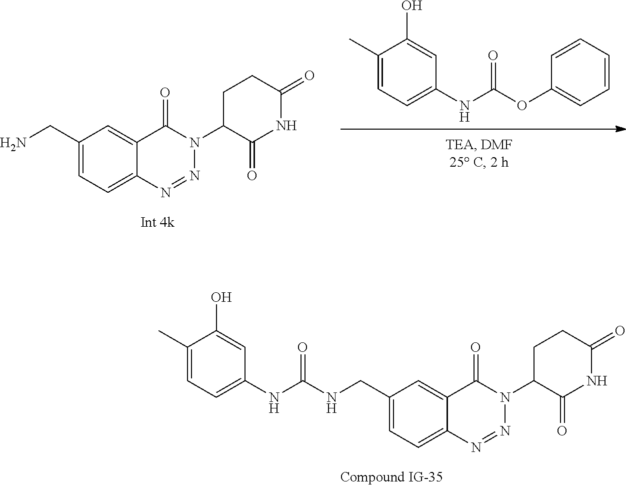 [SMILES: NCc1ccc2nnn(C3CCC(=O)NC3=O)c(=O)c2c1 ; Cc1ccc(NC(=O)Oc2ccccc2)cc1O ; Cc1ccc(NC(=O)NCc2ccc3nnn(C4CCC(=O)NC4=O)c(=O)c3c2)cc1O]

To a solution of 3-(6-(aminomethyl)-4-oxobenzo[d][1,2,3]triazin-3(4H)-yl)piperidine-2,6-dione, Int 4k, (60.0 mg, 209 μmol, 1.00 eq) in dimethyl formamide (1.00 mL) was added triethylamine (63.4 mg, 627 μmol, 87.2 μL, 3.00 eq) and phenyl (3-hydroxy-4-m ethylphenyl)carbamate (50.8 mg, 209 μmol, 1.00 eq). The mixture was stirred at 25° C. for 2 h. The reaction mixture was concentrated under reduced pressure to give a residue. The residue was purified by Prep-HPLC (column: YMC-Actus Triart C18 150*30 mm*7 um; mobile phase: [water(FA)-ACN];B %: 23%-53%, 10 min). Further purified by reversed-phase HPLC (column: spherical C18, 20-45 um, 100 Å, SW 40, mobile phase: [water(0.1% Formic Acid)-ACN]) to give 1-((3-(2,6-dioxopiperidin-3-yl)-4-oxo-3,4-dihydrobenzo[d][1,2,3]triazin-6-yl)methyl)-3-(3-hydroxy-4-methylphenyl) urea, Compound IG-35, (23.0 mg, 52.6 μmol, 25% yield) as a white solid. ¹H NMR (400 MHz, DMSO-d₆) δ=11.19 (br s, 1H), 9.13 (s, 1H), 8.53 (s, 1H), 8.23 (d, J=8.4 Hz, 1H), 8.17 (s, 1H), 8.08-8.02 (m, 1H), 7.04 (d, J=2.0 Hz, 1H), 6.86 (d, J=8.0 Hz, 1H), 6.80-6.75 (m, 1H), 6.64 (dd, J=2.0, 8.0 Hz, 1H), 6.03-5.94 (m, 1H), 4.51 (d, J=5.6 Hz, 2H), 2.99-2.90 (m, 1H), 2.70 (br d, J=5.2 Hz, 1H), 2.44 (br dd, J=3.6, 8.0 Hz, 1H), 2.29-2.24 (m, 1H), 2.01 (s, 3H). MS (ESI) m/z 437.2 [M+H]⁺

### Example 18

### Synthesis of 1-(3-chloro-4-methylphenyl)-3-((2-(2,6-dioxopiperidin-3-yl)-6-hydroxy-1-oxoisoindolin-5-yl)methyl)urea (Compound IG-36)

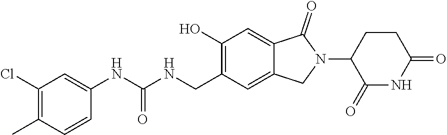 [SMILES: Cc1ccc(NC(=O)NCc2cc3c(cc2O)C(=O)N(C2CCC(=O)NC2=O)C3)cc1Cl]

### Step A. Preparation of Int 41

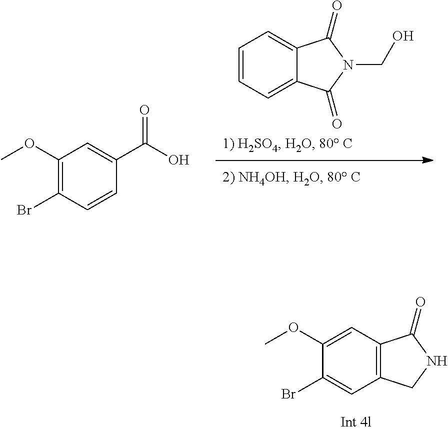

To a solution of 2-(hydroxymethyl)isoindoline-1,3-dione (4.60 g, 26.0 mmol, 1.20 eq) and 4-bromo-3-methoxybenzoic acid (5.00 g, 21.6 mmol, 1.00 eq) in sulfuric acid (20.0 mL) was heated to 80° C. and stirred for 3 h. The mixture was cooled to 25° C. and poured into ice water. The suspension was stirred for 15 min and filtered, the yellow solid was washed by water (20.0 mL×3). Then the solid was dissolved in ammonium hydroxide (30.0 mL, 28%) and ethyl alcohol (30.0 mL) and heated to 80° C. for 3 h. The mixture was concentrated under reduced pressure to give a residue. The residue was triturated with water (50.0 mL) at 25° C. for 15 min and collected by filtration to give crude product. Then further triturated with acetonitrile (50.0 mL) at 25° C. for 15 min. The solid was collected by filtration and dried in vacuo to give 5-bromo-6-methoxyisoindolin-1-one, Int 41, (4.00 g, 11.6 mmol, 53% yield, 70% purity) as a white solid. MS (ESI) m/z 244.0 [M+H]⁺

### Step B. Preparation of Int 4m

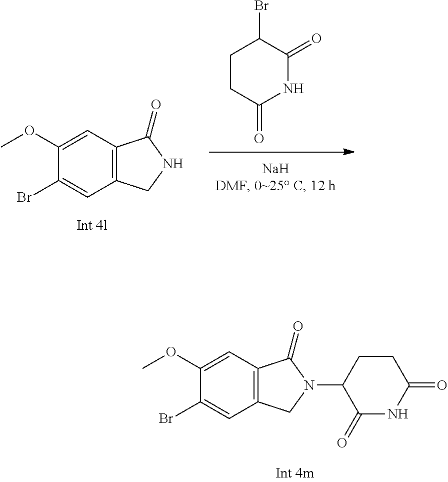 [SMILES: COc1cc2c(cc1Br)CNC2=O ; O=C1CCC(Br)C(=O)N1 ; COc1cc2c(cc1Br)CN(C1CCC(=O)NC1=O)C2=O]

To a solution of 5-bromo-6-methoxyisoindolin-1-one, Int 41, (1.00 g, 4.13 mmol, 1.00 eq) in dimethyl formamide (5.00 mL) was added 3-bromopiperidine-2,6-dione (3.97 g, 20.66 mmol, 5.00 eq) and sodium hydride (1.65 g, 41.3 mmol, 60% purity, 10.0 eq) under nitrogen atmosphere at 0° C. The mixture was stirred at 25° C. for 12 h. The reaction was quenched with formic acid (10% in water 100 mL). The mixture was extracted with ethyl acetate (3×100 mL). The combined organic extracts were washed with brine (200 mL), dried over sodium sulfate and filtered. The filtrate was concentrated under reduced pressure to give a residue. The residue was purified by prep-HPLC (0.1% formic acid conditions). The desired fraction was collected and lyophilized to afford 3-(5-bromo-6-methoxy-1-oxoisoindolin-2-yl)piperidine-2,6-dione, Int 4m, (1.50 g, 4.16 mmol, 33% yield, 98% purity) as a white solid. ¹H NMR (400 MHz, DMSO-d₆) δ=10.99 (s, 1H), 7.95 (s, 1H), 7.89 (s, 1H), 7.36 (s, 1H), 5.11 (dd, J=5.2, 13.4 Hz, 1H), 4.42-4.35 (m, 1H), 4.29-4.24 (m, 1H), 3.94 (s, 3H), 2.96-2.89 (m, 1H), 2.62-2.56 (m, 1H), 2.45-2.35 (m, 1H), 2.05-1.99 (m, 1H).

### Step C. Preparation of Int 4n

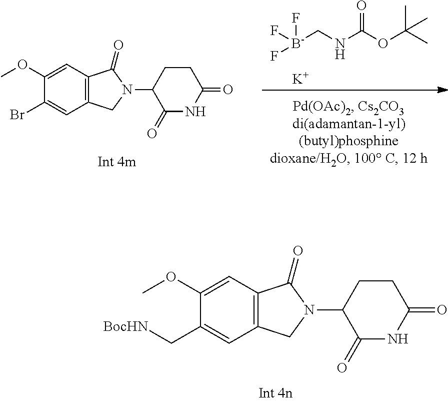 [SMILES: COc1cc2c(cc1Br)CN(C1CCC(=O)NC1=O)C2=O ; CC(C)(C)OC(=O)NC[B-](F)(F)F ; [K+] ; COc1cc2c(cc1CNC(=O)OC(C)(C)C)CN(C1CCC(=O)NC1=O)C2=O]

To a solution of 3-(5-bromo-6-methoxy-1-oxoisoindolin-2-yl)piperidine-2,6-dione, Int 4m, (400 mg, 1.13 mmol, 1.00 eq) in dioxane (10.0 mL) and water (2.00 mL) was added cesium carbonate (738 mg, 2.27 mmol, 2.00 eq), bis(1-adamantyl)-butyl-phosphane (162 mg, 453 μmol, 0.400 eq), potassium;(tert-butoxycarbonylamino)methyl-trifluoro-boranide (269 mg, 1.13 mmol, 1.00 eq) and palladium(II) acetate (51 mg, 227 μmol, 0.200 eq) under nitrogen atmosphere. The mixture was stirred at 100° C. for 12 h under nitrogen atmosphere. The reaction mixture was cooled to 25° C. Ethyl acetate (40.0 mL) and water (40.0 mL) were added and layers were separated. The aqueous phase was extracted with ethyl acetate (2×30.0 mL). Combined extracts were washed with brine (40.0 mL), dried over sodium sulfate and filtered. The filtrate was concentrated under reduced pressure to give a residue. The residue was purified by prep-HPLC (formic acid 0.1% conditions). The desired fraction was concentrated under reduced pressure to give tert-butyl ((2-(2,6-dioxopiperidin-3-yl)-6-methoxy-1-oxoisoindolin-5-yl)methyl)carbamate, Int 4n, (70.0 mg, 174 μmol, 7% yield) as a white solid. ¹H NMR (400 MHz, DMSO-d₆) δ=10.97 (s, 1H), 7.33 (s, 1H), 7.23 (s, 1H), 5.10 (dd, J=5.6, 13.4 Hz, 1H), 4.42-4.33 (m, 1H), 4.26-4.20 (m, 1H), 4.17 (br d, J=6.0 Hz, 2H), 3.88 (s, 3H), 2.98-2.85 (m, 1H), 2.59 (br dd, J=3.2, 16.9 Hz, 1H), 2.40-2.35 (m, 1H), 2.03-1.97 (m, 1H), 1.71 (br s, 1H), 1.40 (s, 9H).

### Step D. Preparation of Int 4o

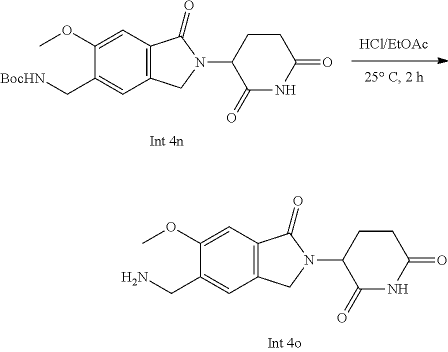 [SMILES: COc1cc2c(cc1CNC(=O)OC(C)(C)C)CN(C1CCC(=O)NC1=O)C2=O ; COc1cc2c(cc1CN)CN(C1CCC(=O)NC1=O)C2=O]

To a solution of tert-butyl ((2-(2,6-dioxopiperidin-3-yl)-6-methoxy-1-oxoisoindolin-5-yl)methyl)carbamate, Int 4n, (70.0 mg, 174 μmol, 1.00 eq) in hydrochloric acid/dioxane (4M, 2.00 mL)). The mixture was stirred at 25° C. for 1 h. The mixture was concentrated under reduced pressure to give 3-(5-(aminomethyl)-6-methoxy-1-oxoisoindolin-2-yl)piperidine-2,6-dione, Int 4o, (50.0 mg, 165 μmol, 95% yield) as a white solid. MS (ESI) m/z 287.2 [M+H]P

### Step E. Preparation of Int 4p

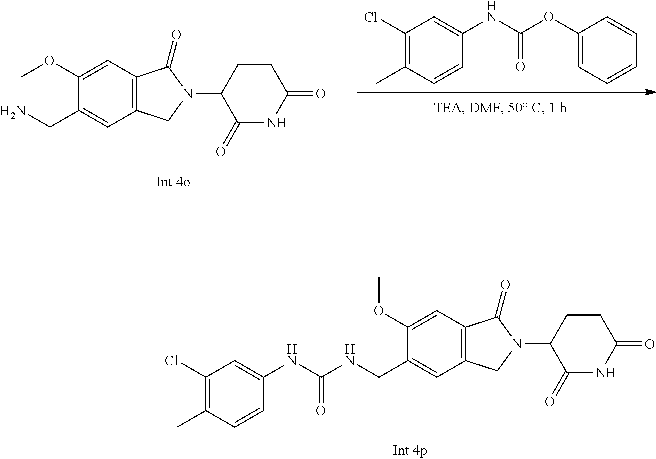 [SMILES: COc1cc2c(cc1CN)CN(C1CCC(=O)NC1=O)C2=O ; Cc1ccc(NC(=O)Oc2ccccc2)cc1Cl ; COc1cc2c(cc1CNC(=O)Nc1ccc(C)c(Cl)c1)CN(C1CCC(=O)NC1=O)C2=O]

To a solution of 3-(5-(aminomethyl)-6-methoxy-1-oxoisoindolin-2-yl)piperidine-2,6-dione, Int 4o, (45.0 mg, 148 gmol, 1.00 eq) in dimethyl formamide (2.00 mL) was added triethylamine (60.1 mg, 593 μmol, 82.6 μL, 4.00 eq) and phenyl (3-chloro-4-methylphenyl)carbamate (42.7 mg, 163 μmol, 1.10 eq). The mixture was stirred at 50° C. for 1 h. The mixture was concentrated under reduced pressure to give a residue. The residue was purified by prep-HPLC (0.1% formic acid conditions). The desired fraction was collected and lyophilized to afford 1-(3-chloro-4-methylphenyl)-3-((2-(2,6-dioxopiperidin-3-yl)-6-methoxy-1-oxoisoindolin-5-yl)methyl) urea, Int 4p, (25 mg, 53.1 μmol, 35% yield) as a white solid. MS (ESI) m/z 471.3 [M+H]⁺

### Step F. Preparation of Compound IG-36

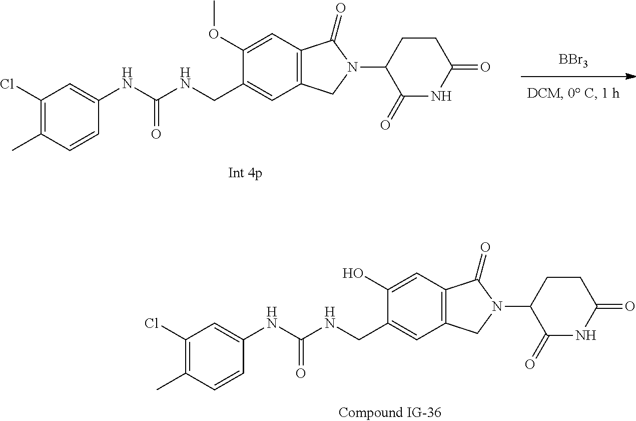 [SMILES: COc1cc2c(cc1CNC(=O)Nc1ccc(C)c(Cl)c1)CN(C1CCC(=O)NC1=O)C2=O ; "BBr3" ; Cc1ccc(NC(=O)NCc2cc3c(cc2O)C(=O)N(C2CCC(=O)NC2=O)C3)cc1Cl]

To a solution of 1-(3-chloro-4-methylphenyl)-3-((2-(2,6-dioxopiperidin-3-yl)-6-methoxy-1-oxoisoindolin-5-yl)methyl)urea, Int 4p, (22.0 mg, 46.7 μmol, 1.00 eq) in dichloromethane (1.00 mL) was added tribromoborane (117 mg, 467 μmol, 45.0 μL, 10.0 eq). The mixture was stirred at 0° C. for 1 h. The reaction was quenched with water (5.00 mL) and the reaction mixture was filtered to give filter cake. The filter cake was purified by prep-HPLC (column: Phenomenex luna C18 150*25 mm*10 um; mobile phase: [water(FA)-ACN];gradient: 25%-55% B over 10 min). The desired fraction was collected and lyophilized to afford 1-(3-chloro-4-methylphenyl)-3-((2-(2,6-dioxopiperidin-3-yl)-6-hydroxy-1-oxoisoindolin-5-yl)methyl)urea, Compound IG-36, (2.18 mg, 4.77 μmol, 10% yield) as a white solid. ¹H NMR (400 MHz, DMSO-d₆) δ=10.96 (s, 1H), 10.11 (s, 1H), 8.77 (s, 1H), 7.65 (d, J=2.0 Hz, 1H), 7.35 (s, 1H), 7.19-7.15 (m, 1H), 7.13-7.06 (m, 2H), 6.67-6.56 (m, 1H), 5.05 (dd, J=5.2, 13.7 Hz, 1H), 4.37-4.25 (m, 3H), 4.22-4.14 (m, 1H), 2.94-2.84 (m, 1H), 2.60 (br d, J=2.8 Hz, 1H), 2.37-2.32 (m, 1H), 2.22 (s, 3H), 2.01-1.95 (m, 1H). MS (ESI) m/z 457.3 [M+H]⁺

The compounds in Table 11 were prepared in a manner similar to that described for Compound IG-36 using Int 4o and the appropriately substituted aryl isocyanate, or phenyl carbamate (c.f. Example 2).

TABLE 11

| IG | | |
|---|---|---|
| No. | Structure | ¹H NMR/MS |
| 49 | 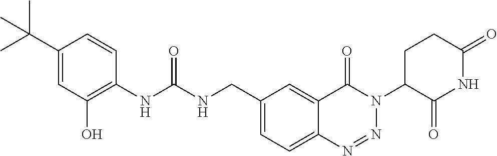 [SMILES: CC(C)(C)c1ccc(NC(=O)NCc2ccc3nnn(C4CCC(=O)NC4=O)c(=O)c3c2)c(O)c1] | |
| | 1-(4-(tert-butyl)-2-hydroxyphenyl)-3-((3-(2,6- | |
| | dioxopiperidin-3-yl)-4-oxo-3,4- | |
| | dihydrobenzo[d][1,2,3]triazin-6-yl)methyl)urea | |

### Example 19

### Synthesis of 1-(3-chloro-4-methylphenyl)-3-((2-(2,6-dioxopiperidin-3-yl)-6-(methylamino)-1-oxoisoindolin-5-yl)methyl)urea (Compound IG-45)

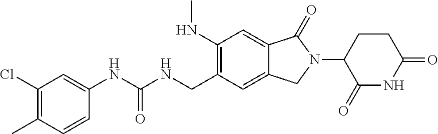 [SMILES: CNc1cc2c(cc1CNC(=O)Nc1ccc(C)c(Cl)c1)CN(C1CCC(=O)NC1=O)C2=O]

### Step A. Preparation of Int 5a

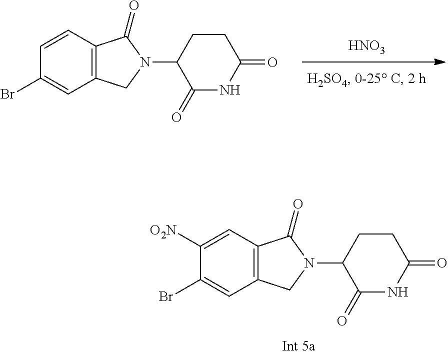 [SMILES: O=C1CCC(N2Cc3cc(Br)ccc3C2=O)C(=O)N1 ; "HNO3" ; O=C1CCC(N2Cc3cc(Br)c([N+](=O)[O-])cc3C2=O)C(=O)N1]

To a solution of 3-(5-bromo-1-oxo-isoindolin-2-yl)piperidine-2,6-dione (3.00 g, 9.28 mmol, 1.00 eq) in sulfuric acid (25.0 mL) was added a solution of nitric acid (12.5 g, 129 mmol, 8.93 mL, 65.0% purity, 13.9 eq) in sulfuric acid (16.0 mL) dropwise at 0° C. under nitrogen atmosphere. The mixture was stirred at 25° C. for 2 h. The mixture was added to ice water (50.0 mL) dropwise then filtered, the filter cake was washed with water (5.00 mL×3) and dried in vacuo. The solid was triturated with ethyl acetate (10.0 mL) at 25° C. to afford 3-((5-bromo-2-methoxyphenyl)amino)propanoic acid, Int 5a, (3.20 g, 8.69 mmol, 93% yield) as a brown solid. ¹H NMR (400 MHz, DMSO-d₆) δ=11.05 (s, 1H), 8.35 (s, 1H), 8.25 (s, 1H), 5.15 (dd, J=5.2, 13.2 Hz, 1H), 4.62-4.43 (m, 2H), 2.96-2.87 (m, 1H), 2.61 (br d, J=18.0 Hz, 1H), 2.47-2.38 (m, 1H), 2.08-2.01 (m, 1H).

### Step B. Preparation of Int 5b

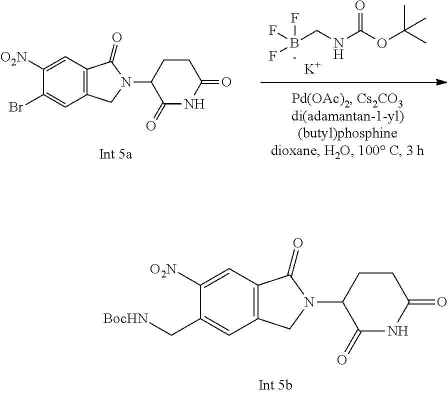

To a solution of 3-(5-bromo-6-nitro-1-oxoisoindolin-2-yl)piperidine-2,6-dione, Int 5a, (1.90 g, 5.16 mmol, 1.00 eq) in dioxane (100 mL) was added water (10.0 mL), potassium(I)(((tert-butoxycarbonyl) amino) methyl) trifluoroborate (1.35 g, 5.68 mmol, 1.10 eq), bis(1-adamantyl)-butyl-phosphane (740 mg, 2.06 mmol, 0.400 eq), cesium carbonate (3.36 g, 10.3 mmol, 2.00 eq) and palladium(II) acetate (463 mg, 2.06 mmol, 0.400 eq) under nitrogen atmosphere. The mixture was stirred at 100° C. for 3 h. The reaction mixture was concentrated under reduced pressure to remove solvent. The residue was diluted with water (500 mL) and extracted with ethyl acetate (500 mL×3). The combined organic layers were concentrated under reduced pressure to give a residue. The residue was purified by flash silica gel chromatography (ISCO®, 40 g SepaFlash® Silica Flash Column, Eluent of 0~80% Ethyl acetate/Petroleum ether gradient @100 mL/min) to give tert-butyl ((2-(2,6-dioxopiperidin-3-yl)-6-nitro-1-oxoisoindolin-5-yl)methyl)carbamate, Int 5b, (600 mg, 1.43 mmol, 28% yield) as a yellow solid. ¹H NMR (400 MHz, DMSO-d₆) δ=11.04 (s, 1H), 8.26 (s, 1H), 7.76 (s, 1H), 7.57 (br t, J=6.0 Hz, 1H), 5.16 (dd, J=5.2, 13.2 Hz, 1H), 4.67-4.59 (m, 1H), 4.53-4.44 (m, 3H), 2.95-2.88 (m, 1H), 2.64 (br s, 1H), 2.41 (br dd, J=4.0, 13.2 Hz, 1H), 2.07-2.03 (m, 1H), 1.40 (s, 9H).

### Step C. Preparation of Int 5c

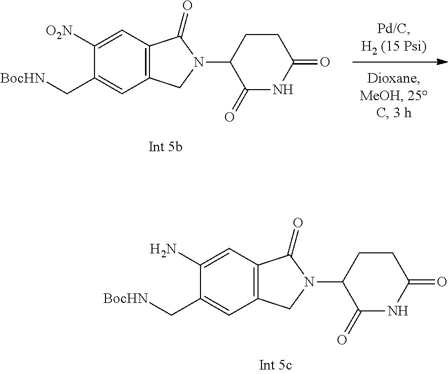 [SMILES: CC(C)(C)OC(=O)NCc1cc2c(cc1[N+](=O)[O-])C(=O)N(C1CCC(=O)NC1=O)C2 ; CC(C)(C)OC(=O)NCc1cc2c(cc1N)C(=O)N(C1CCC(=O)NC1=O)C2]

To a solution of tert-butyl ((2-(2,6-dioxopiperidin-3-yl)-6-nitro-1-oxoisoindolin-5-yl)methyl)carbamate, Int 5b, (600 mg, 1.43 mmol, 1.00 eq) in dioxane (20.0 mL) were added methanol (20.0 mL) and wet palladium on carbon (100 mg, 10% purity) under nitrogen atmosphere. The suspension was degassed under vacuum and purged with hydrogen several times. The mixture was stirred under hydrogen (15 psi) at 25° C. for 3 h. The reaction mixture was filtered. The filtrate was concentrated to give tert-butyl ((6-amino-2-(2,6-dioxopiperidin-3-yl)-1-oxoisoindolin-5-yl)methyl)carbamate, Int 5c, (300 mg, crude) as a yellow solid. MS (ESI) m/z 333.1 [M+H-56]⁺

### Step D. Preparation of Int 5d

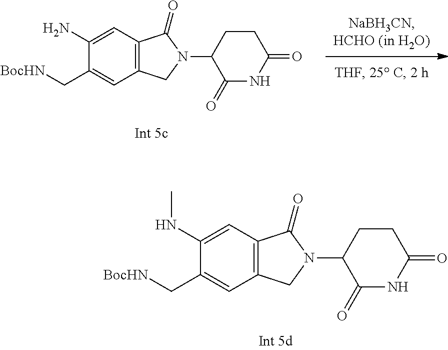 [SMILES: CC(C)(C)OC(=O)NCc1cc2c(cc1N)C(=O)N(C1CCC(=O)NC1=O)C2 ; CNc1cc2c(cc1CNC(=O)OC(C)(C)C)CN(C1CCC(=O)NC1=O)C2=O]

To a solution of tert-butyl ((6-amino-2-(2,6-dioxopiperidin-3-yl)-1-oxoisoindolin-5-yl)methyl)carbamate, Int 5c, (150 mg, 386 μmol, 1.00 eq) in tetrahydrofuran (5.00 mL) were added formaldehyde solution (313 mg, 3.86 mmol, 287 μL, 37% purity, 10.0 eq) and sodium cyanoborohydride (72.8 mg, 1.16 mmol, 3.00 eq). The mixture was stirred at 25° C. for 2 h. The reaction mixture was concentrated under reduced pressure to give a residue. The residue was purified by prep-HPLC (column: Phenomenex Luna C18 150*25 mm*10 um; mobile phase: [water(FA)-ACN]; gradient: 20%-50% B over 10 min) to give tert-butyl ((2-(2,6-dioxopiperidin-3-yl)-6-(methylamino)-1-oxoisoindolin-5-yl)methyl)carbamate, Int 5d, (100 mg, 156 μmol, 41% yield, 63% purity) as a white solid. MS (ESI) m/z 403.2 [M+H]⁺

### Step E. Preparation of Int 5e

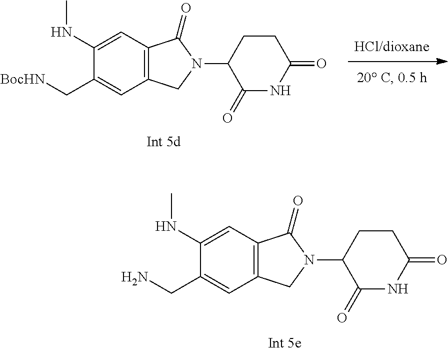 [SMILES: CNc1cc2c(cc1CNC(=O)OC(C)(C)C)CN(C1CCC(=O)NC1=O)C2=O ; CNc1cc2c(cc1CN)CN(C1CCC(=O)NC1=O)C2=O]

To a solution of tert-butyl ((2-(2,6-dioxopiperidin-3-yl)-6-(methylamino)-1-oxoisoindolin-5-yl)methyl) carbamate, Int 5d, (20.0 mg, 49.7 μmol, 1.00 eq) in ethyl acetate (2.00 mL) was added hydrochloric acid/ethyl acetate (4 M, 5.00 mL). The mixture was stirred at 20° C. for 0.5 h The reaction mixture was concentrated under reduced pressure to give 3-(5-(aminomethyl)-6-(methylamino)-1-oxoisoindolin-2-yl)piperidine-2,6-dione, Int 5e, (10.0 mg, crude) as a white solid. MS (ESI) m/z 286.1 [M+H-17]⁺

### Step F. Preparation of Compound IG-45

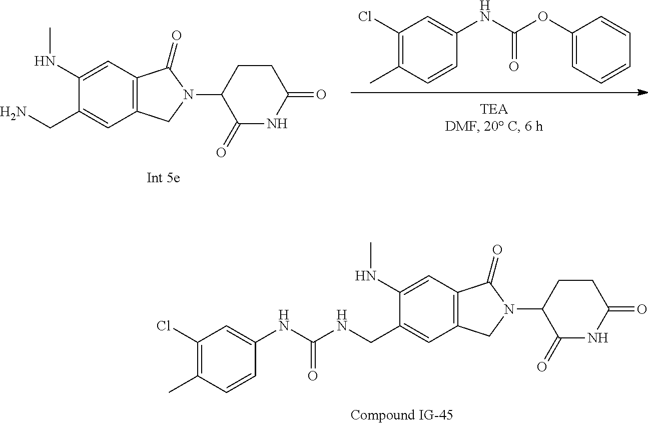 [SMILES: CNc1cc2c(cc1CN)CN(C1CCC(=O)NC1=O)C2=O ; Cc1ccc(NC(=O)Oc2ccccc2)cc1Cl ; CNc1cc2c(cc1CNC(=O)Nc1ccc(C)c(Cl)c1)CN(C1CCC(=O)NC1=O)C2=O]

To a solution of 3-(5-(aminomethyl)-6-(methylamino)-1-oxoisoindolin-2-yl)piperidine-2,6-dione, Int 5e, (10.0 mg, 33.1 μmol, 1.00 eq) in dimethyl formamide (1.00 mL) were added triethylamine (10.0 mg, 99.2 μmol, 13.8 μL, 3.00 eq), phenyl (3-chloro-4-methylphenyl)carbamate (10.4 mg, 39.7 μmol, 1.20 eq). The mixture was stirred at 20° C. for 6 h. The reaction mixture was concentrated under reduced pressure to give residue. The residue was purified by prep-HPLC (column: Phenomenex Luna C18 150*25 mm*10 um; mobile phase: [water(FA)-ACN];gradient: 32%-62% B over 10 min). The desired fraction was collected and lyophilized to afford 1-(3-chloro-4-methylphenyl)-3-((2-(2,6-dioxopiperidin-3-yl)-6-(methylamino)-1-oxoisoindolin-5-yl)methyl)urea, Compound IG-45, (4.40 mg, 9.27 μmol, 28% yield, 99% purity) as a white solid ¹H NMR (400 MHz, DMSO-d₆) δ=10.96 (s, 1H), 8.87 (s, 1H), 7.65 (d, J=2.0 Hz, 1H), 7.28 (s, 1H), 7.20-7.17 (m, 1H), 7.15-7.12 (m, 1H), 6.76 (s, 1H), 6.73 (br t, J=6.0 Hz, 1H), 5.68 (q, J=4.4 Hz, 1H), 5.08 (dd, J=4.8, 13.4 Hz, 1H), 4.32-4.27 (m, 1H), 4.24 (br d, J=5.6 Hz, 2H), 4.19-4.13 (m, 1H), 2.80 (d, J=4.8 Hz, 3H), 2.61-2.56 (m, 1H), 2.36 (br dd, J=4.4, 13.2 Hz, 1H), 2.23 (s, 4H), 2.01-1.98 (m, 1H). MS (ESI) m/z 470.3 [M+H]⁺

### Example 20

### Synthesis of N-(2-(2,6-dioxopiperidin-3-yl)-6-hydroxy-1,3-dioxoisoindolin-5-yl)-2-(trifluoromethoxy)benzenesulfonamide (Compound IG-46)

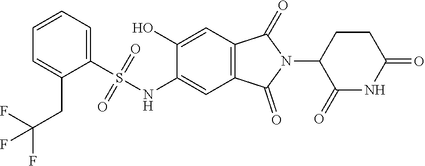 [SMILES: O=C1CCC(N2C(=O)c3cc(O)c(NS(=O)(=O)c4ccccc4CC(F)(F)F)cc3C2=O)C(=O)N1]

### Step A. Preparation of Int 4q

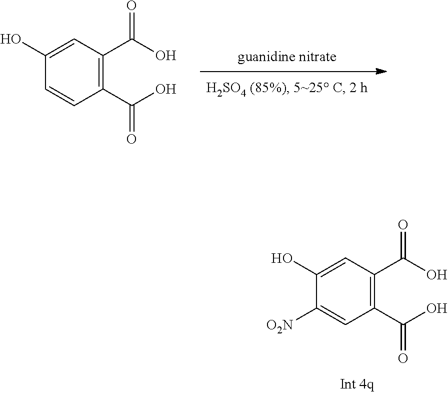 [SMILES: O=C(O)c1ccc(O)cc1C(=O)O ; O=C(O)c1cc(O)c([N+](=O)[O-])cc1C(=O)O]

To a suspension of 4-hydroxyphthalic acid (10.0 g, 54.9 mmol, 1.00 eq) in sulfuric acid (200 mL, 85% purity) was slowly added guanidine nitrate (6.57 g, 53.8 mmol, 0.980 eq) at 5° C. Then the mixture was stirred at 25° C. for another 2 h. Then the reaction solution was poured into ice-cold water (500 mL) and extracted with ethyl acetate (500 mL). The organic layer was separated and concentrated under reduced pressure to afford 4-hydroxy-5-nitro-phthalic acid, Int 4q, (11.1 g, 29.3 mmol, 53% yield, 60% purity) as a yellow solid. ¹H NMR (400 MHz, DMSO-d₆) δ=12.09 (br s, 2H), 8.28 (s, 1H), 7.20 (s, 1H).

### Step B. Preparation of Int 4r

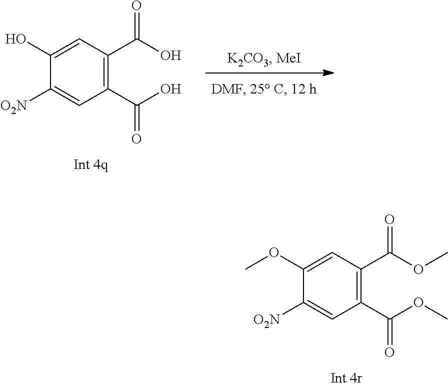

To a solution of 4-hydroxy-5-nitro-phthalic acid, Int 4q, (2.00 g, 8.81 mmol, 1.00 eq) in dimethyl formamide (20.0 mL) was added methyl iodide (7.50 g, 52.8 mmol, 3.29 mL, 6.00 eq) and potassium carbonate (3.65 g, 26.4 mmol, 3.00 eq). Then the mixture was stirred at 25° C. for 12 h. The reaction mixture was diluted with water (100 mL) and extracted with ethyl acetate (30.0 mL). The combined organic layers were washed with brine (20 mL), dried over anhydrous sodium sulfate, filtered and concentrated under reduced pressure to give a residue. The residue was purified by flash silica gel chromatography (ISCO®; 40 g SepaFlash® Silica Flash Column, Eluent of 0~40% ethyl acetate/petroleum ether gradient @60 mL/min) to give dimethyl 4-methoxy-5-nitro-benzene-1,2-dicarboxylate, Int 4r, (900 mg, 3.34 mmol, 37% yield) as a white solid. ¹H NMR (400 MHz, DMSO-d₆) δ=8.36 (s, 1H), 7.63 (s, 1H), 4.04 (s, 3H), 3.87 (s, 3H), 3.84 (s, 3H).

### Step C. Preparation of Int 4s

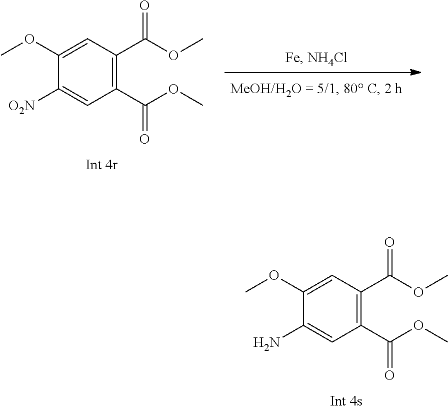

To a solution of dimethyl 4-methoxy-5-nitro-benzene-1,2-dicarboxylate, Int 4r, (900 mg, 3.34 mmol, 1.00 eq) in methyl alcohol (10.0 mL) and water (5.00 mL) was added iron (933 mg, 16.7 mmol, 5.00 eq) and ammonium chloride (894 mg, 16.7 mmol, 5.00 eq). The mixture was stirred at 80° C. for 2 h. After the reaction mixture was partitioned between saturated sodium bicarbonate aqueous solution (15.0 mL) and ethyl acetate (10.0 mL×3). The combined organic phase was separated, dried over anhydrous sodium sulfate filtered and concentrated under reduced pressure to afford dimethyl 4-amino-5-methoxy-benzene-1,2-dicarboxylate, Int 4s, (420 mg, 1.76 mmol, 52% yield) as a gray solid. ¹H NMR (400 MHz, DMSO-d₆) δ=7.14 (s, 1H), 6.77 (s, 1H), 5.72 (s, 2H), 3.84 (s, 3H), 3.73 (d, J=3.2 Hz, 6H).

### Step D. Preparation of Int 4t

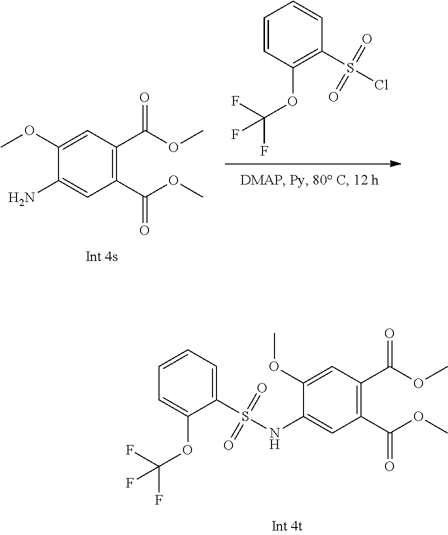

To a solution of dimethyl 4-amino-5-methoxy-benzene-1,2-dicarboxylate, Int 4s, (400 mg, 1.67 mmol, 1.00 eq) in pyridine (5.00 mL) was added dimethylaminopyridine (20.4 mg, 167 μmol, 0.10 eq) and 2-(trifluoromethoxy)benzenesulfonyl chloride (523 mg, 2.01 mmol, 1.20 eq). The mixture was stirred at 80° C. for 12 h. The reaction mixture was partitioned between water (15.0 mL) and ethyl acetate (10.0 mL×3). The organic phase was separated, dried over anhydrous sodium sulfate filtered and concentrated under reduced pressure to afford dimethyl 4-methoxy-5-((2-(trifluoromethoxy)phenyl)sulfonamido)phthalate, Int 4t, (590 mg, 1.27 mmol, 76% yield) as a white solid. ¹H NMR (400 MHz, DMSO-d₆) δ=10.14 (s, 1H), 7.86 (dd, J=1.6, 7.9 Hz, 1H), 7.79-7.74 (m, 1H), 7.67 (s, 1H), 7.57-7.48 (m, 2H), 7.18 (s, 1H), 3.78 (d, J=2.0 Hz, 6H), 3.62 (s, 3H).

### Step E. Preparation of Int 4u

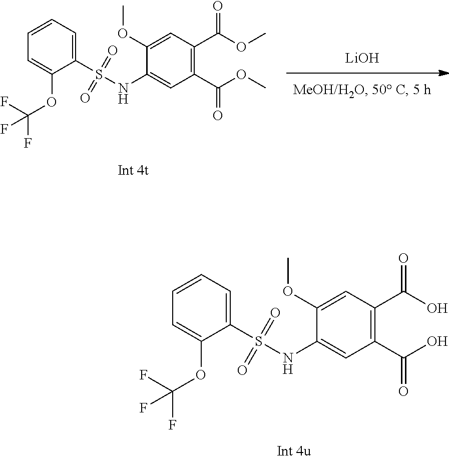 [SMILES: COC(=O)c1cc(NS(=O)(=O)c2ccccc2OC(F)(F)F)c(OC)cc1C(=O)OC ; "LiOH" ; COc1cc(C(=O)O)c(C(=O)O)cc1NS(=O)(=O)c1ccccc1OC(F)(F)F]

To a solution of dimethyl 4-methoxy-5-((2-(trifluoromethoxy)phenyl)sulfonamido)phthalate, Int 4t, (590 mg, 1.27 mmol, 1.00 eq) in methyl alcohol (5.00 mL) and water (5.00 mL) was added lithium hydroxide (244 mg, 10.2 mmol, 8.00 eq). The mixture was stirred at 50° C. for 5 h. After the reaction mixture was partitioned between water (15.0 mL) and ethyl acetate (10.0 mL×3). The combined organic phase was separated, dried over anhydrous sodium sulfate filtered and concentrated under reduced pressure to afford 4-methoxy-5-((2-(trifluoromethoxy)phenyl)sulfonamido)phthalic acid, Int 4u, (320 mg, 735 μmol, 57% yield) as a yellow solid. ¹H NMR (400 MHz, DMSO-d₆) δ=13.12 (br s, 2H), 10.02 (s, 1H), 7.83 (dd, J=1.2, 8.0 Hz, 1H), 7.79-7.73 (m, 1H), 7.62 (s, 1H), 7.57 (br d, J=8.0 Hz, 1H), 7.49 (t, J=7.6 Hz, 1H), 7.10 (s, 1H), 3.59 (s, 3H).

### Step F. Preparation of Int 4v

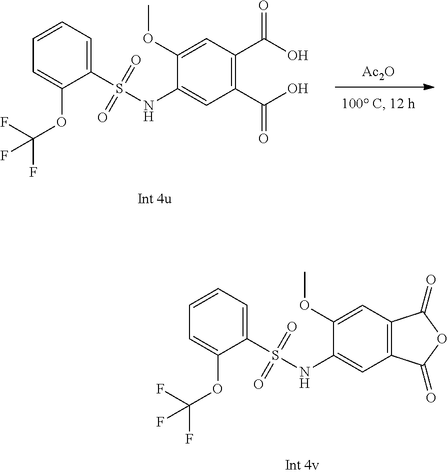 [SMILES: "Ac2O" ; COc1cc(C(=O)O)c(C(=O)O)cc1NS(=O)(=O)c1ccccc1OC(F)(F)F ; COc1cc2c(cc1NS(=O)(=O)c1ccccc1OC(F)(F)F)C(=O)OC2=O]

To a solution of 4-methoxy-5-((2-(trifluoromethoxy)phenyl)sulfonamido)phthalic acid (320 mg, 735 μmol, 1.00 eq) in acetic anhydride, Int 4u, (5.00 mL) was degassed and purged with nitrogen for 3 times and then the mixture was stirred at 100° C. for 12 h under nitrogen atmosphere. After the reaction mixture was concentrated under reduced pressure to give N-(6-methoxy-1,3-dioxo-1,3-dihydroisobenzofuran-5-yl)-2-(trifluoromethoxy) benzenesulfonamide, Int 4v, (460 mg, crude) as a white solid. MS (ESI) m/z 418.1 [M+H]⁺

### Step G. Preparation of Int 4w

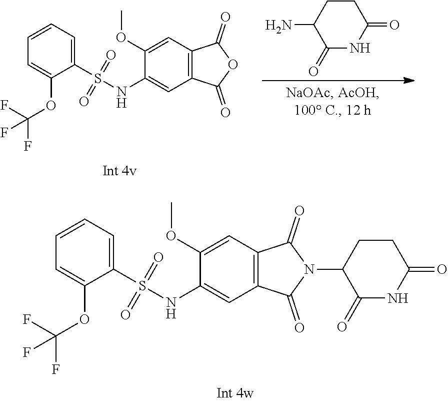 [SMILES: COc1cc2c(cc1NS(=O)(=O)c1ccccc1OC(F)(F)F)C(=O)OC2=O ; NC1CCC(=O)NC1=O ; COc1cc2c(cc1NS(=O)(=O)c1ccccc1OC(F)(F)F)C(=O)N(C1CCC(=O)NC1=O)C2=O]

To a solution of N-(6-methoxy-1,3-dioxo-isobenzofuran-5-yl)-2-(trifluoromethoxy)benzenesulfonamide, Int 4v, (306 mg, 733 μmol, 1.00 eq) in acetic acid (5.00 mL) was added sodium acetate (120 mg, 1.47 mmol, 2.00 eq), 3-aminopiperidine-2,6-dione;hydrochloride (121 mg, 733 μmol, 1.00 eq). The mixture was stirred at 100° C. for 12 h under nitrogen atmosphere. After the reaction mixture was concentrated under reduced pressure to give a residue. The residue was purified by reversed-phase HPLC (0.1% FA) and lyophilized to afford N-(2-(2,6-dioxopiperidin-3-yl)-6-methoxy-1,3-dioxoisoindolin-5-yl)-2-(trifluoromethoxy)benzenesulfonamide, Int 4w, (150 mg, 284 μmol, 38% yield) as a white solid. ¹H NMR (400 MHz, DMSO-d₆) δ=10.94 (br s, 1H), 8.21 (d, J=8.0 Hz, 1H), 8.01 (s, 1H), 7.90 (t, J=8.0 Hz, 1H), 7.73 (s, 1H), 7.64 (d, J=7.6 Hz, 1H), 7.53-7.44 (m, 1H), 5.15 (dd, J=5.6, 12.8 Hz, 1H), 3.98 (s, 3H), 2.93-2.84 (m, 1H), 2.67-2.57 (m, 2H), 2.15-2.09 (m, 1H).

### Step H. Preparation of Compound IG-46

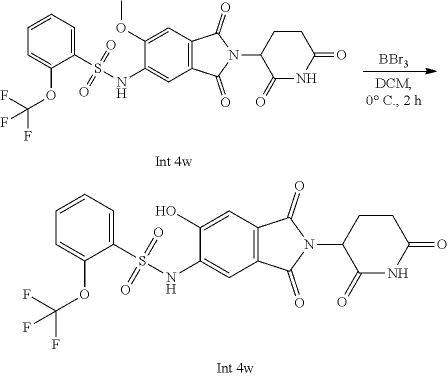

To a solution of N-(2-(2,6-dioxopiperidin-3-yl)-6-methoxy-1,3-dioxoisoindolin-5-yl)-2-(trifluoromethoxy) benzenesulfonamide, Int 4w, (130 mg, 246 μmol, 1.00 eq) in dichloromethane (2.00 mL) was added boron tribromide (617 mg, 2.46 mmol, 236 μL, 10.0 eq) at 0° C. Then the mixture was stirred at 0° C. for 2 h. The reaction mixture was poured into ice water and filtered. The filter cake was purified by prep-HPLC (column: Waters Xbridge 150*25 mm*5 um; mobile phase: [water (NH₄HCO₃)-ACN]; gradient: 18%-48% B over 9 min) and prep-HPLC (column: Phenomenex Luna C18 150*25 mm*10 um; mobile phase: [water (FA)-ACN]; gradient: 28%-58% B over 10 min) to afford N-(2-(2,6-dioxopiperidin-3-yl)-6-hydroxy-1,3-dioxoisoindolin-5-yl)-2-(trifluoromethoxy)benzenesulfonamide, Compound IG-46, (6.65 mg, 12.4 μmol, 5% yield, 96% purity) as a yellow solid. ¹H NMR (400 MHz, DMSO-d₆) δ=11.08 (s, 1H), 7.94 (dd, J=1.6, 7.9 Hz, 1H), 7.79-7.73 (m, 1H), 7.61 (s, 1H), 7.58-7.48 (m, 2H), 7.15 (s, 1H), 5.04 (dd, J=5.6, 12.8 Hz, 1H), 2.91-2.80 (m, 1H), 2.59 (br d, J=2.8 Hz, 1H), 2.46 (br d, J=4.4 Hz, 1H), 2.04-1.96 (m, 1H). MS (ESI) m/z 514.2 [M+H]⁺

### Example L-3: Synthesis of 4-((S)-2-((S)-2-(3-(2,5-dioxo-2,5-dihydro-1H-pyrrol-1-yl)propanamido)propanamido)propanamido)benzyl (2-(2-chloro-4-(3-(2-(2,6-dioxopiperidin-3-yl)-1-oxoisoindolin-5-yl)propanamido)phenethoxy)ethyl) (methyl)carbamate (IG-L-3)

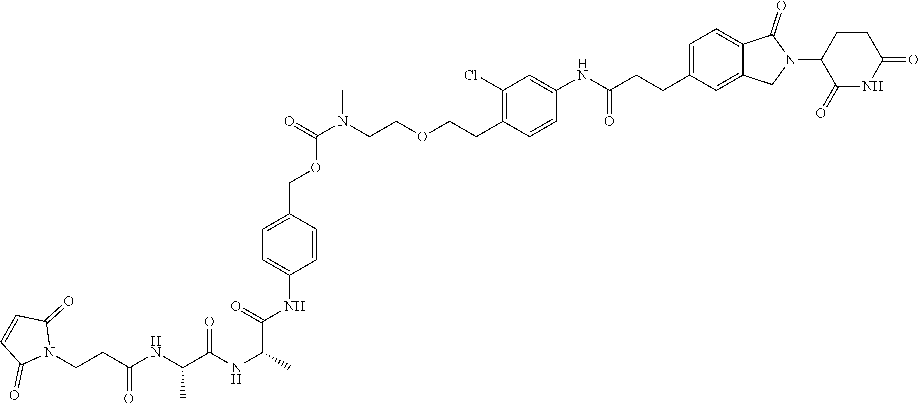 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)CCc3ccc4c(c3)CN(C3CCC(=O)NC3=O)C4=O)cc2Cl)cc1]

### Preparation of Compound IG-L-3

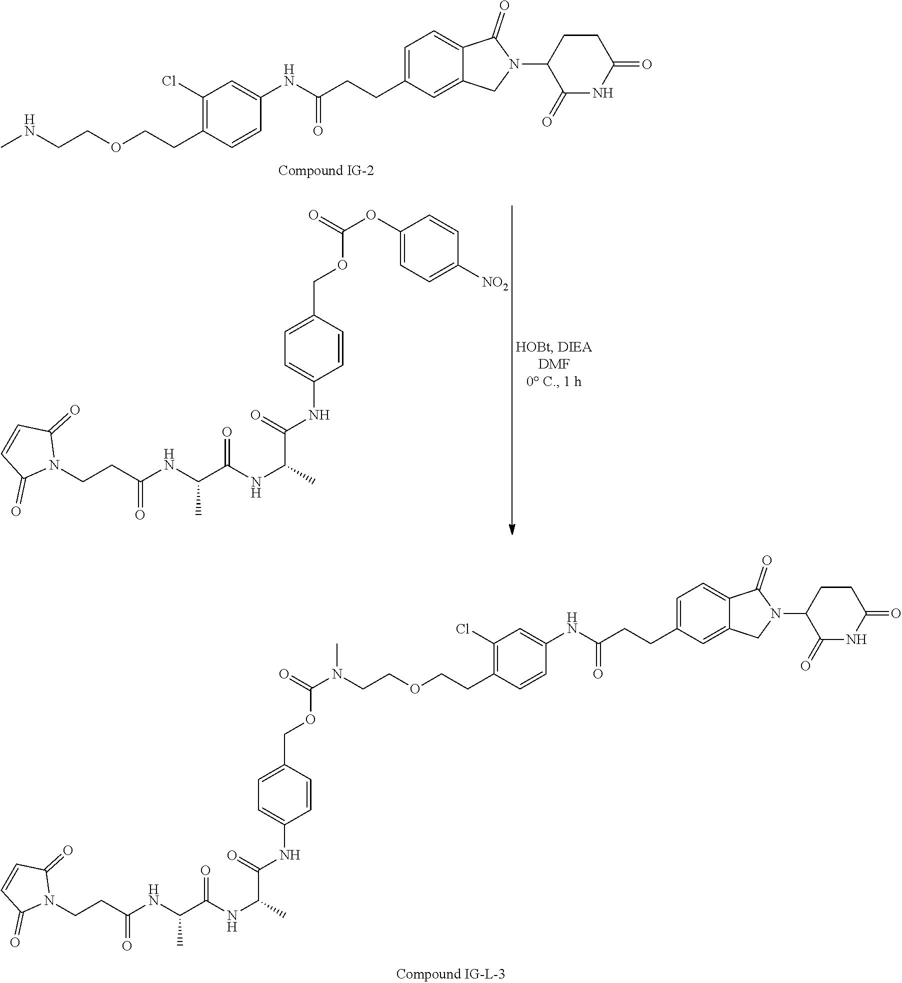 [SMILES: CNCCOCCc1ccc(NC(=O)CCc2ccc3c(c2)CN(C2CCC(=O)NC2=O)C3=O)cc1Cl ; C[C@H](NC(=O)CCN1C(=O)C=CC1=O)C(=O)N[C@@H](C)C(=O)Nc1ccc(COC(=O)Oc2ccc([N+](=O)[O-])cc2)cc1 ; C[C@H](NC(=O)CCN1C(=O)C=CC1=O)C(=O)N[C@@H](C)C(=O)Nc1ccc(COC(=O)N(C)CCOCCc2ccc(NC(=O)CCc3ccc4c(c3)CN(C3CCC(=O)NC3=O)C4=O)cc2Cl)cc1]

To a solution of N-(3-chloro-4-(2-(2-(methylamino)ethoxy)ethyl)phenyl)-3-(2-(2,6-dioxopiperidin-3-yl)-1-oxoisoindolin-5-yl)propenamide, Compound IG-2, (100 mg, 190 umol, 1.00 eq) and 4-((S)-2-((S)-2-(3-(2,5-dioxo-2,5-dihydro-1H-pyrrol-1-yl)propanamido)propanamido)propanamido)benzyl (4-nitrophenyl) carbonate (110 mg, 190 umol, 1.00 eq) in NN-dimethylformamide (0.5 mL) was added 1-hydroxybenzotriazole (25.6 mg, 190 umol, 1.00 eq) and diisopropylethylamine (73.6 mg, 569 umol, 99.2 uL, 3.00 eq), the mixture was stirred at 0° C. for 1 h. The mixture was filtered. The filtrate was purified by Prep-HPLC (column: Unisil 3-100 C18 Ultra 150*50 mm*3 um; mobile phase: [water(FA)-ACN];B %: 35%-65%, 7 min) and lyophilized to afford 4-((S)-2-((S)-2-(3-(2,5-dioxo-2,5-dihydro-1H-pyrrol-1-yl)propanamido)propanamido)propanamido)benzyl (2-(2-chloro-4-(3-(2-(2,6-dioxopiperidin-3-yl)-1-oxoisoindolin-5-yl)propanamido)phenethoxy)ethyl) (methyl)carbamate, Compound IG-L-3, (52.96 mg, 54.1 umol, 29% yield, 99% purity) as a white solid. ¹H NMR (400 MHz, DMSO-d₆) δ=10.97 (br s, 1H), 10.05 (s, 1H), 9.85 (s, 1H), 8.20 (d, J=7.2 Hz, 1H), 8.11 (d, J=7.1 Hz, 1H), 7.78 (d, J=1.8 Hz, 1H), 7.62 (dd, J=8.0, 16.2 Hz, 3H), 7.48 (s, 1H), 7.40 (d, J=8.0 Hz, 1H), 7.37-7.17 (m, 4H), 6.99 (s, 2H), 5.10 (dd, J=5.1, 13.2 Hz, 1H), 4.98 (s, 2H), 4.50-4.11 (m, 4H), 3.60 (t, J=7.3 Hz, 2H), 3.56-3.47 (m, 3H), 3.37-3.34 (m, 2H), 3.02 (br t, J=7.4 Hz, 2H), 2.96-2.86 (m, 2H), 2.82 (br d, J=8.4 Hz, 4H), 2.67 (br t, J=7.6 Hz, 3H), 2.62-2.60 (m, 1H), 2.45-2.35 (m, 3H), 2.03-1.93 (m, 1H), 1.30 (d, J=7.2 Hz, 3H), 1.17 (d, J=7.2 Hz, 3H)

The compounds in Table 12 were prepared in a manner similar to that described for Compound IG-L-3 using the appropriate compound as starting material.

TABLE 12

| IG-L | | |
|---|---|---|
| No. | Structure | ¹H NMR/MS |
| IG-L-1 | 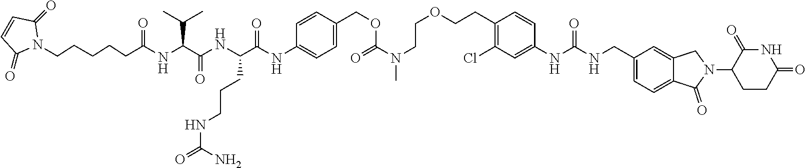 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1] | ¹H NMR (400 MHz, DMSO-d₆) δ = 10.97 (s, 1H), 9.99 (s, 1H), 8.89-8.76 (m, 1H), 8.08 (br d, J = 7.6 Hz, 1H), 7.80 (d, J = 8.8 Hz, 1H), 7.71-7.64 (m, 2H), 7.58 (br d, J = 8.8 Hz, 2H), 7.51 (s, 1H), 7.44 (d, J = 7.6 Hz, 1H), 7.28 (br d, J = 7.2 Hz, 2H), 7.20-7.09 (m, 2H), 6.99 (s, 2H), 6.90-6.74 (m, 1H), 5.10 (dd, J = 5.0, 13.4 Hz, 1H), 4.98 (s, 2H), 4.47-4.28 (m, 5H), 4.22-4.16 (m, 1H), 3.54 (br s, 2H), 3.49 (br dd, J = 4.6, 6.7 Hz, 3H), 3.40-3.31 (m, 4H), 3.09-2.87 (m, 3H), 2.83 (br d, J = 8.4 Hz, 5H), 2.63-2.55 (m, 1H), 2.54 (br s, 1H), 2.41-2.35 (m, 2H), 2.20-2.06 (m, 2H), 2.02-1.91 (m, 2H), 1.74-1.64 (m, 1H), 1.62- |
| | | 1.54 (m, 1H), 1.52-1.39 (m, 5H), 1.38-1.29 (m, |
| | | 1H), 1.23-1.11 (m, 2H), 0.83 (dd, J = 6.8, 12.3 Hz, |
| | | 6H). |
| | | MS (ESI) m/z 1128.6 [M + 3]⁺ |
| IG-L-2 | 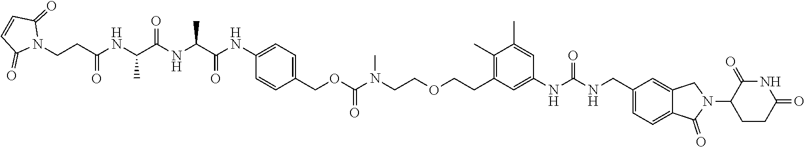 [SMILES: Cc1cc(NC(=O)NCc2ccc3c(c2)CN(C2CCC(=O)NC2=O)C3=O)cc(CCOCCN(C)C(=O)OCc2ccc(NC(=O)[C@H](C)NC(=O)[C@H](C)NC(=O)CCN3C(=O)C=CC3=O)cc2)c1C] | ¹H NMR (400 MHz, DMSO-d₆) δ = 10.97 (s, 1H), 9.84 (s, 1H), 8.35 (s, 1H), 8.20 (d, J = 7.0 Hz, 1H), 8.10 (d, J = 7.1 Hz, 1H), 7.68 (d, J = 7.8 Hz, 1H), 7.60 (br d, J = 8.3 Hz, 2H), 7.50 (s, 1H), 7.43 (d, J = 7.7 Hz, 1H), 7.29 (br d, J = 8.3 Hz, 2H), 7.11-7.02 (m, 2H), 6.99 (s, 2H), 6.66 (br t, J = 6.0 Hz, 1H), 5.10 (dd, J = 5.2, 13.3 Hz, 1H), 4.98 (s, 2H), 4.49-4.28 (m, 5H), 4.23 (br t, J = 7.1 Hz, 1H), 3.61 (br t, J = 7.3 Hz, 2H), 3.49 (br d, J = 6.1 Hz, 4H), 3.36 (br d, J = 5.4 Hz, 2H), 2.96-2.88 (m, 1H), 2.84 (br d, J = 5.1 |
| | | Hz, 3H), 2.74 (br s, 2H), 2.61 (br d, J = 2.4 Hz, 1H), |
| | | 2.45-2.34 (m, 3H), 2.15 (s, 3H), 2.10-2.02 (m, 3H), |
| | | 2.02-1.93 (m, 1H), 1.30 (d, J = 7.1 Hz, 3H), 1.17 (d, |
| | | J = 7.1 Hz, 3H). |
| | | MS (ESI) m/z 964.6 [M + H]⁺ |
| IG-L-4 | 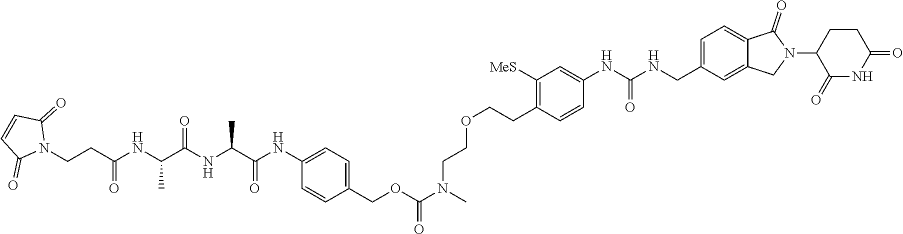 [SMILES: CSc1cc(NC(=O)NCc2ccc3c(c2)CN(C2CCC(=O)NC2=O)C3=O)ccc1CCOCCN(C)C(=O)OCc1ccc(NC(=O)[C@H](C)NC(=O)[C@H](C)NC(=O)CCN2C(=O)C=CC2=O)cc1] | ¹H NMR (400 MHz, DMSO-d₆) δ = 10.97 (s, 1H), 9.86 (s, 1H), 8.70 (s, 1H), 8.21 (d, J = 6.9 Hz, 1H), 8.13-8.08 (m, 1H), 7.69 (d, J = 7.9 Hz, 1H), 7.60 (br d, J = 8.3 Hz, 2H), 7.51 (s, 1H), 7.44 (d, J = 8.0 Hz, 1H), 7.39 (d, J = 1.8 Hz, 1H), 7.29 (br d, J = 7.3 Hz, 2H), 7.13-7.06 (m, 1H), 7.06-6.95 (m, 3H), 6.78 (br t, J = 5.9 Hz, 1H), 5.10 (dd, J = 5.1, 13.3 Hz, 1H), 4.99 (s, 2H), 4.48-4.40 (m, 3H), 4.37 (br t, J = 7.2 Hz, 1H), 4.34-4.28 (m, 1H), 4.23 (t, J = 7.0 Hz, 1H), 3.64-3.60 (m, 2H), 3.50 (br s, 4H), 3.39-3.32 (m, 2H), 2.96-2.87 (m, 1H), 2.84 (br d, J = 8.8 Hz, 3H), 2.77 (br s, 2H), 2.63-2.56 (m, 1H), 2.44-2.41 (m, 1H), 2.39 (s, 4H), 2.38-2.31 (m, 1H), 2.03-1.96 (m, 1H), 1.30 (d, J = 7.0 Hz, 3H), 1.17 (d, J = 7.1 Hz, 3H). MS (ESI) m/z 982.6 [M + H]⁺ |
| IG-L-5 | 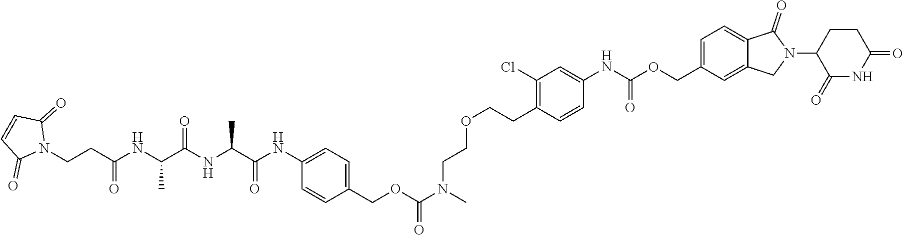 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)OCc3ccc4c(c3)CN(C3CCC(=O)NC3=O)C4=O)cc2Cl)cc1] | ¹H NMR (400 MHz, DMSO-d₆) δ = 10.99 (br s, 1H), 9.96 (s, 1H), 9.84 (s, 1H), 8.20 (d, J = 7.2 Hz, 1H), 8.10 (d, J = 7.6 Hz, 1H), 7.75 (d, J = 7.6 Hz, 1H), 7.65 (s, 1H), 7.62-7.54 (m, 4H), 7.43-7.12 (m, 5H), 6.99 (s, 1H), 5.28 (s, 2H), 5.12 (dd, J = 5.2, 13.3 Hz, 1H), 4.98 (s, 2H), 4.51-4.45 (m, 1H), 4.39-4.32 (m, 2H), 4.23 (t, J = 7.2 Hz, 1H), 3.60 (t, J = 7.4 Hz, 2H), 3.58-3.42 (m, 5H), 3.36-3.34 (m, 2H), 2.96-2.85 (m, 2H), 2.82 (br d, J = 7.0 Hz, 3H), 2.63-2.57 (m, 1H), 2.44-2.34 (m, 3H), 2.05-1.96 (m, 1H), 1.30 (d, J = 7.2 Hz, 3H), 1.17 (d, J = 7.2 Hz, 3H). MS (ESI) m/z 971.5 [M + H]⁺ |
| IG-L-6 | 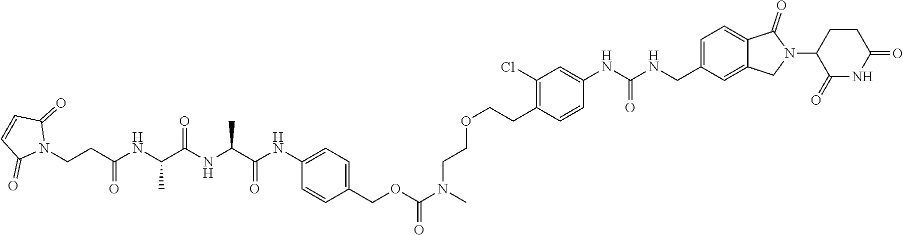 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)NCc3ccc4c(c3)CN(C3CCC(=O)NC3=O)C4=O)cc2Cl)cc1] | ¹H NMR (400 MHz, DMSO-d₆) δ = 10.97 (br d, J = 1.6 Hz, 1H), 9.85 (s, 1H), 8.84 (br d, J = 16.8 Hz, 1H), 8.20 (d, J = 6.8 Hz, 1H), 8.11 (d, J = 7.2 Hz, 1H), 7.71-7.65 (m, 2H), 7.60 (br d, J = 8.4 Hz, 2H), 7.51 (s, 1H), 7.44 (d, J = 7.6 Hz, 1H), 7.29 (br d, J = 7.6 Hz, 2H), 7.20-7.12 (m, 2H), 6.99 (s, 2H), 6.93- 6.83 (m, 1H), 5.10 (dd, J = 5.2, 13.3 Hz, 1H), 4.98 (s, 2H), 4.47-4.31 (m, 5H), 4.28-4.19 (m, 1H), 3.61 (t, J = 7.2 Hz, 2H), 3.51 (br dd, J = 6.4, 15.7 Hz, 4H), 3.37-3.34 (m, 2H), 2.98-2.85 (m, 2H), 2.83 (br d, J = 8.2 Hz, 4H), 2.62-2.56 (m, 1H), 2.45-2.34 (m, 3H), 2.04-1.95 (m, 1H), 1.30 (d, J = 7.2 Hz, 3H), 1.17 (d, J = 7.2 Hz, 3H). MS (ESI) m/z 970.5 [M + H]⁺ |
| IG-L-7 | 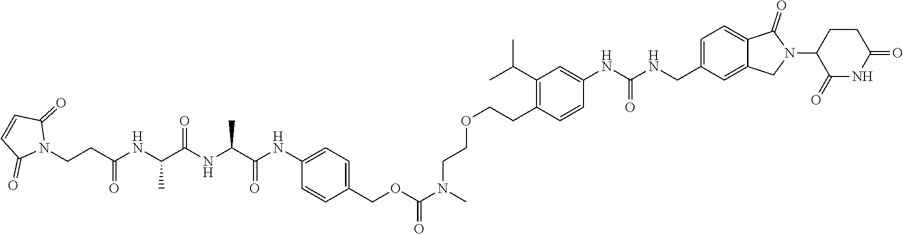 [SMILES: CC(C)c1cc(NC(=O)NCc2ccc3c(c2)CN(C2CCC(=O)NC2=O)C3=O)ccc1CCOCCN(C)C(=O)OCc1ccc(NC(=O)[C@H](C)NC(=O)[C@H](C)NC(=O)CCN2C(=O)C=CC2=O)cc1] | ¹H NMR (400 MHz, DMSO-d₆) δ = 10.98 (s, 1H), 9.85 (s, 1H), 8.52 (s, 1H), 8.20 (d, J = 7.2 Hz, 1H), 8.10 (d, J = 7.2 Hz, 1H), 7.69 (d, J = 7.6 Hz, 1H), 7.60 (br d, J = 8.4 Hz, 2H), 7.51 (s, 1H), 7.43 (d, J = 7.6 Hz, 1H), 7.32-7.25 (m, 3H), 7.15 (br d, J = 8.4 Hz, 1H), 7.01-6.93 (m, 3H), 6.64 (br t, J = 5.6 Hz, 1H), 5.10 (dd, J = 5.1, 13.2 Hz, 1H), 4.99 (s, 2H), 4.45-4.19 (m, 6H), 3.60 (t, J = 7.2 Hz, 2H), 3.51- 3.42 (m, 5H), 3.28-2.93 (m, 2H), 2.93-2.80 (m, 4H), 2.78-2.70 (m, 2H), 2.63-2.54 (m, 1H), 2.43- 2.34 (m, 3H), 2.05-1.94 (m, 1H), 1.30 (d, J = 7.2 Hz, 3H), 1.17 (d, J = 7.2 Hz, 3H), 1.13 (br d, J = 6.8 Hz, 6H). MS (ESI) m/z 978.3 [M + H]⁺ |
| IG-L-9 | 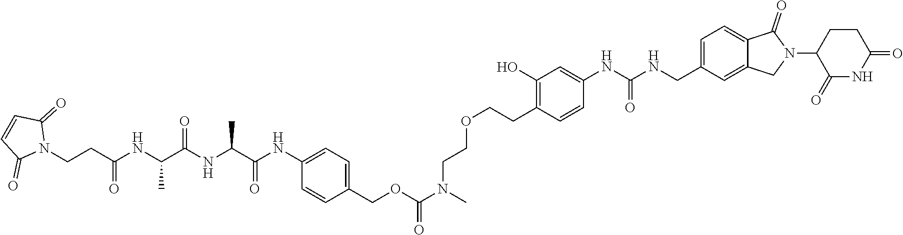 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)NCc3ccc4c(c3)CN(C3CCC(=O)NC3=O)C4=O)cc2O)cc1] | ¹H NMR (400 MHz, DMSO-d₆) δ = 10.98 (s, 1H), 9.85 (s, 1H), 9.20 (s, 1H), 8.49-8.42 (m, 1H), 8.21 (br d, J = 7.0 Hz, 1H), 8.11 (br d, J = 7.2 Hz, 1H), 7.69 (d, J = 8.0 Hz, 1H), 7.60 (br d, J = 8.0 Hz, 2H), 7.51 (s, 1H), 7.44 (d, J = 8.0 Hz, 1H), 7.30 (br d, J = 7.8 Hz, 2H), 7.06 (d, J = 1.6 Hz, 1H), 6.99 (s, 2H), 6.91-6.84 (m, 1H), 6.65 (br d, J = 7.6 Hz, 2H), 5.10 (dd, J = 5.0, 13.2 Hz, 1H), 4.99 (s, 2H), 4.51-4.31 (m, 5H), 4.29-4.21 (m, 1H), 3.61 (t, J = 7.2 Hz, 2H), 3.47 (br d, J = 1.2 Hz, 4H), 3.37-3.35 (m, 2H), 2.96- 2.83 (m, 4H), 2.64 (br d, J = 8.0 Hz, 2H), 2.60-2.54 (m, 1H), 2.43-2.36 (m, 3H), 2.07-1.93 (m, 1H), 1.30 (br d, J = 7.2 Hz, 3H), 1.18 (d, J = 7.2 Hz, 3H). MS (ESI) m/z 952.3 [M + H]⁺ |
| IG-L-10 | 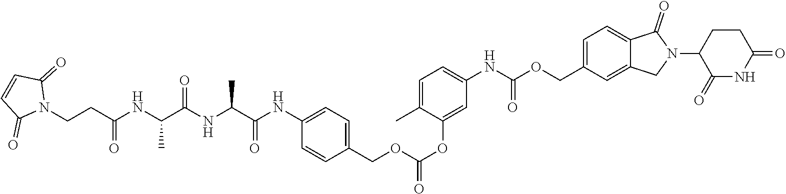 [SMILES: Cc1ccc(NC(=O)OCc2ccc3c(c2)CN(C2CCC(=O)NC2=O)C3=O)cc1OC(=O)OCc1ccc(NC(=O)[C@H](C)NC(=O)[C@H](C)NC(=O)CCN2C(=O)C=CC2=O)cc1] | ¹H NMR (400 MHz, DMSO-d6) δ = 10.99 (br s, 1H), 9.91 (s, 2H), 8.22 (d, J = 6.8 Hz, 1H), 8.13 (d, J = 7.2 Hz, 1H), 7.79 (s, 1H), 7.70-7.62 (m, 4H), 7.40 (d, J = 8.4 Hz, 2H), 7.34 (s, 1H), 7.27-7.22 (m, 1H), 7.21- 7.17 (m, 1H), 7.00 (s, 2H), 5.28-5.20 (m, 4H), 5.13 (dd, J = 5.2, 13.4 Hz, 1H), 4.50-4.32 (m, 3H), 4.28- 4.21 (m, 1H), 3.62 (t, J = 7.2 Hz, 2H), 2.65-2.56 (m, 2H), 2.44-2.38 (m, 3H), 2.04 (s, 4H), 1.32 (d, J = 7.2 Hz, 3H), 1.19 (d, J = 7.2 Hz, 3H). MS (ESI) m/z 866.4 [M + H]⁺ |
| IG-L-11 | 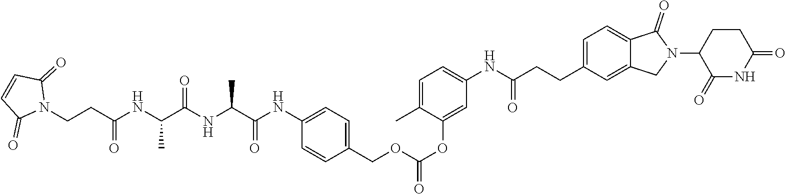 [SMILES: Cc1ccc(NC(=O)CCc2ccc3c(c2)CN(C2CCC(=O)NC2=O)C3=O)cc1OC(=O)OCc1ccc(NC(=O)[C@H](C)NC(=O)[C@H](C)NC(=O)CCN2C(=O)C=CC2=O)cc1] | ¹H NMR (400 MHz, DMSO-d₆) δ = 10.98 (s, 1H), 10.03 (s, 1H), 9.92 (s, 1H), 8.22 (d, J = 6.8 Hz, 1H), 8.13 (d, J = 6.8 Hz, 1H), 7.65 (t, J = 8.4 Hz, 3H), 7.52- 7.47 (m, 2H), 7.40 (d, J = 8.4 Hz, 3H), 7.29 (dd, J = 1.6, 8.0 Hz, 1H), 7.21-7.17 (m, 1H), 7.00 (s, 2H), 5.21 (s, 2H), 5.09 (dd, J = 5.6, 13.2 Hz, 1H), 4.45- 4.19 (m, 4H), 3.61 (t, J = 7.2 Hz, 2H), 3.02 (br t, J = 7.6 Hz, 2H), 2.97-2.85 (m, 1H), 2.66 (br t, J = 7.6 Hz, 2H), 2.64-2.55 (m, 1H), 2.46-2.36 (m, 3H), 2.04 (s, 3H), 2.01-1.95 (m, 1H), 1.32 (d, J = 6.8 Hz, 3H), 1.18 (d, J = 7.2 Hz, 3H). MS (ESI) m/z 864.4 [M + H]⁺ |
| IG-L-12 | 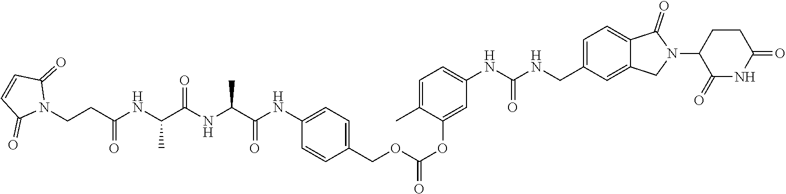 [SMILES: Cc1ccc(NC(=O)NCc2ccc3c(c2)CN(C2CCC(=O)NC2=O)C3=O)cc1OC(=O)OCc1ccc(NC(=O)[C@H](C)NC(=O)[C@H](C)NC(=O)CCN2C(=O)C=CC2=O)cc1] | ¹H NMR (400 MHz, DMSO-d₆) δ = 10.97 (s, 1H), 9.90 (s, 1H), 8.73 (s, 1H), 8.21 (d, J = 6.9 Hz, 1H), 8.11 (d, J = 7.1 Hz, 1H), 7.72-7.63 (m, 3H), 7.51 (s, 1H), 7.44 (d, J = 7.9 Hz, 1H), 7.41-7.36 (m, 3H), 7.15-7.07 (m, 2H), 7.00 (s, 2H), 6.76 (br t, J = 6.1 Hz, 1H), 5.20 (s, 2H), 5.10 (dd, J = 5.0, 13.3 Hz, 1H), 4.47-4.27 (m, 5H), 4.23 (br t, J = 7.2 Hz, 1H), 3.61 (t, J = 7.3 Hz, 2H), 2.96-2.84 (m, 1H), 2.60 (br d, J = 16.5 Hz, 1H), 2.41 (br d, J = 7.8 Hz, 2H), 2.36 (br d, J = 4.5 Hz, 1H), 2.01 (s, 3H), 2.00-1.96 (m, 1H), 1.31 (d, J = 7.1 Hz, 3H), 1.18 (d, J = 7.1 Hz, 3H). MS (ESI) m/z 865.3 [M + H]⁺ |
| IG-L-17 | 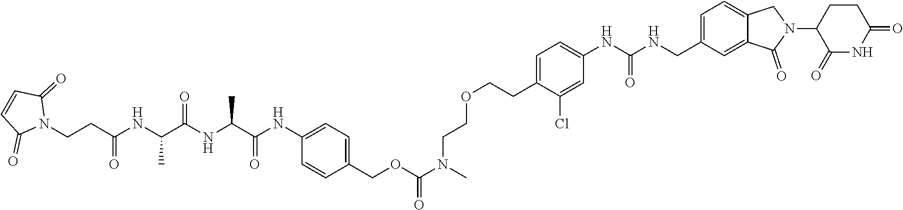 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)NCc3ccc4c(c3)C(=O)N(C3CCC(=O)NC3=O)C4)cc2Cl)cc1] | ¹H NMR (400 MHz, DMSO-d₆) δ = 10.97 (s, 1H), 9.84 (s, 1H), 8.77 (s, 1H), 8.20 (br d, J = 7.2 Hz, 1H), 8.10 (br d, J = 7.2 Hz, 1H), 7.66 (s, 2H), 7.60 (br d, J = 8.4 Hz, 2H), 7.56 (s, 2H), 7.29 (br d, J = 7.2 Hz, 2H), 7.20-7.11 (m, 2H), 6.99 (s, 2H), 6.82 (br t, J = 6.0 Hz, 1H), 5.11 (dd, J = 5.2, 13.3 Hz, 1H), 4.98 (s, 2H), 4.48-4.30 (m, 5H), 4.28-4.21 (m, 1H), 3.61 (t, J = 7.2 Hz, 2H), 3.56-3.46 (m, 4H), 3.35 (br t, J = 5.8 Hz, 2H), 2.96-2.87 (m, 1H), 2.83 (br d, J = 6.4 Hz, 5H), 2.59 (br d, J = 18.4 Hz, 1H), 2.40 (br t, J = 7.2 Hz, 3H), 2.03-1.96 (m, 1H), 1.30 (d, J = 7.2 Hz, 3H), 1.18 (d, J = 7.2 Hz, 3H). MS (ESI) m/z 970.1 [M + H]⁺ |
| | 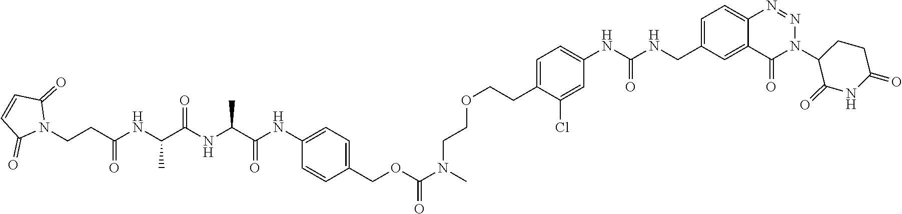 [SMILES: C[C@H](NC(=O)CCN1C(=O)C=CC1=O)C(=O)N[C@@H](C)C(=O)Nc1ccc(COC(=O)N(C)CCOCCc2ccc(NC(=O)NCc3ccc4nnn(C5CCC(=O)NC5=O)c(=O)c4c3)cc2Cl)cc1] | 984.4 |
| IG-L-13 | 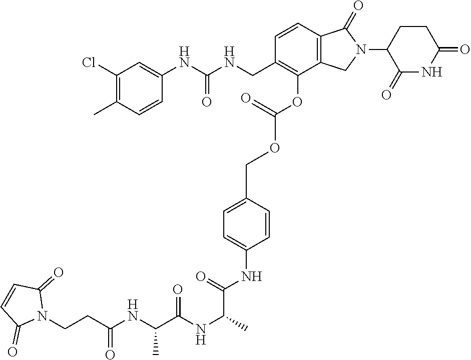 [SMILES: Cc1ccc(NC(=O)NCc2ccc3c(c2OC(=O)OCc2ccc(NC(=O)[C@H](C)NC(=O)[C@H](C)NC(=O)CCN4C(=O)C=CC4=O)cc2)CN(C2CCC(=O)NC2=O)C3=O)cc1Cl] | ¹H NMR (400 MHz, DMSO-d₆) δ = 10.99 (br s, 1H), 9.91 (s, 1H), 8.80 (s, 1H), 8.22 (br d, J = 6.4 Hz, 1H), 8.13 (br d, J = 7.2 Hz, 1H), 7.72-7.63 (m, 4H), 7.56 (br d, J = 7.6 Hz, 1H), 7.40 (br d, J = 8.4 Hz, 2H), 7.19-7.15 (m, 1H), 7.13-7.09 (m, 1H), 7.00 (s, 2H), 6.71 (br t, J = 5.6 Hz, 1H), 5.24 (br s, 2H), 5.15-5.08 (m, 1H), 4.42-4.34 (m, 4H), 4.31-4.21 (m, 2H), 3.61 (br t, J = 7.2 Hz, 2H), 2.96-2.83 (m, 1H), 2.61 (br d, J = 2.0 Hz, 1H), 2.40 (br t, J = 7.2 Hz, 3H), 2.22 (s, 3H), 2.04 - 1.95 (m, 1H), 1.31 (br d, J = 7.2 Hz, 3H), 1.18 (br d, J = 7.2 Hz, 3H). MS (ESI) m/z 899.2 [M + H]⁺ |
| IG-L-57 | 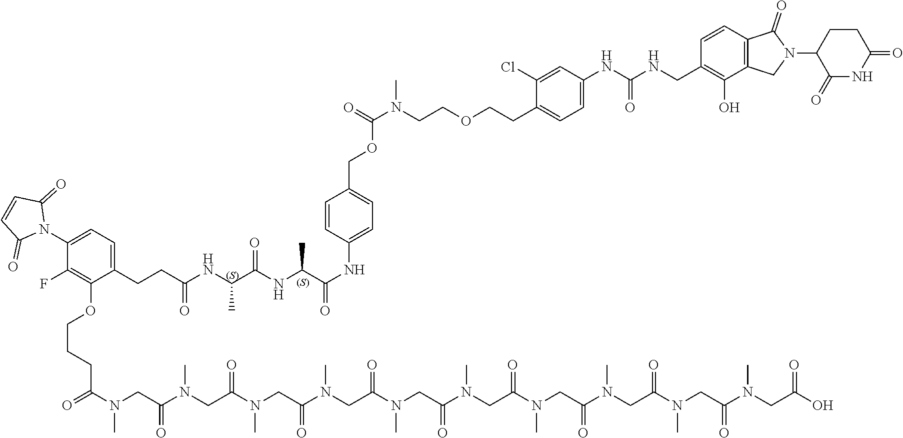 | 1H NMR (400 MHz, DMSO-d6) δ = 11.08-10.88 (m, 1H), 10.01-9.90 (m, 1H), 8.29-8.12 (m, 2H), 7.65 (br s, 1H), 7.60 (br d, J = 7.6 Hz, 2H), 7.35-7.25 (m, 4H), 7.24 (s, 2H), 7.20-7.10 (m, 4H), 7.06-6.99 (m, 1H), 5.08 (dd, J = 5.2, 13.0 Hz, 1H), 4.98 (br s, 2H), 4.39-3.88 (m, 28H), 3.74 (br s, 1H), 3.57-3.44 (m, 5H), 2.96-2.70 (m, 40H), 2.57 (br s, 1H), 2.48- 2.43 (m, 2H), 2.37 (br dd, J = 5.2, 13.2 Hz, 1H), 2.03- 1.96 (m, 1H), 1.96-1.85 (m, 2H), 1.29 (br d, J = 7.2 Hz, 3H), 1.19 (br d, J = 7.2 Hz, 3H). LC-MS (ESI) m/z 947.4[M/2 + H]⁺ |

### Example L-20: Synthesis of 34-(2-(3-(((2S)-1-(((2S)-1-((4-((((2-(2-chloro-4-(3-((2-(2,6-dioxopiperidin-3-yl)-1-oxoisoindolin-5-yl)methyl)ureido)phenethoxy)ethyl) (methyl)carbamoyl)oxy)methyl)phenyl)amino)-1-oxopropan-2-yl)amino)-1-oxopropan-2-yl) amino)-3-oxopropyl)-5-(2,5-dioxo-2,5-dihydro-1H-pyrrol-1-yl)phenoxy)-3,6,9,12,15,18,21,24,27,30-decamethyl-4,7,10,13,16,19,22,25,28,31-decaoxo-3,6,9,12,15,18,21, 24,27,30-decaazatetratriacontanoic acid (IG-L-20)

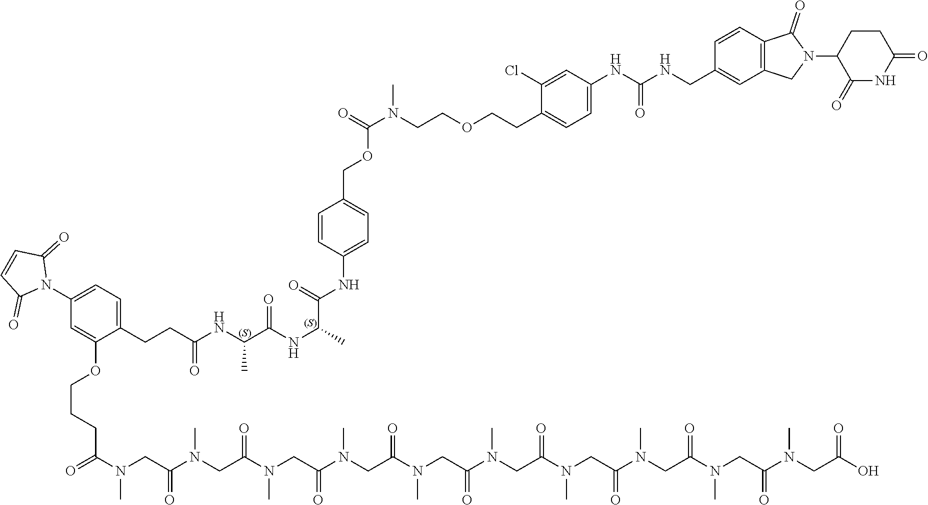

### Step A. Preparation of Int 10a

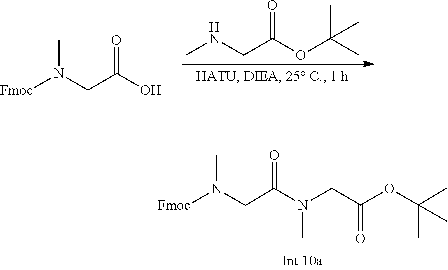

To a solution of tert-butyl methylglycinate (100 g, 550 mmol, 1.00 eq, hydrochloride) and N-(((9H-fluoren-9-yl)methoxy)carbonyl)-N-methylglycine (171 g, 550 mmol, 1.00 eq) in dimethyl formamide (400 mL) was added diisopropylethylamine (142 g, 1.10 μmol, 192 mL, 2.00 eq) and O-(7-azabenzotriazol-1-yl)-N,N,N′,N′-tetramethyluronium hexafluorophosphate (251 g, 661 mmol, 1.20 eq). Then the mixture was stirred at 25° C. for 1 h. The reaction mixture was poured into water (3 L) and extracted with ethyl acetate (3×200 mL). The organic layer was washed with brine (3×200 mL) and concentrated to give a residue. The residue was purified by flash silica gel chromatography (ISCO®; 330 g SepaFlash® Silica Flash Column, Eluent of 30~90% ethyl acetate/petroleum ether gradient @120 mL/min) to afford tert-butyl N′—(N-(((9H-fluoren-9-yl)methoxy)carbonyl)-N-methylglycyl)-N-methylglycinate, Int 10a, (230 g, 524 mmol, 95% yield) as yellow oil. ¹H NMR (400 MHz, DMSO-d₆) δ=7.89 (t, J=8.4 Hz, 2H), 7.69-7.56 (m, 2H), 7.41 (q, J=7.4 Hz, 2H), 7.37-7.25 (m, 2H), 4.33-4.08 (m, 4H), 4.04-3.93 (m, 3H), 2.97-2.79 (m, 6H), 1.45-1.35 (m, 9H).

### Step B. Preparation of Int 10b

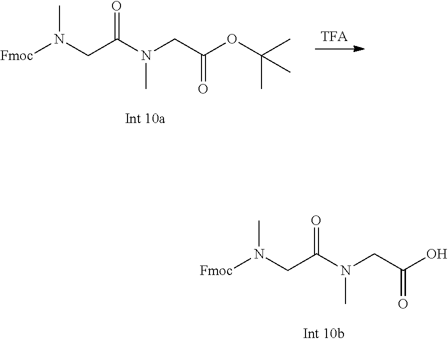

A solution of tert-butyl N—(N-(((9H-fluoren-9-yl)methoxy)carbonyl)-N-methylglycyl)-N-methylglycinate, Int 10a, (230 g, 524 mmol, 1.00 eq) in trifluoroacetic acid (500 mL) and dichloromethane (500 mL) was stirred at 25° C. for 3 h. The reaction mixture was concentrated to give a residue. The residue was purified by flash silica gel chromatography (ISCO®; 330 g SepaFlash® Silica Flash Column, Eluent of 50~100% ethyl acetate/petroleum ether gradient @120 mL/min) to afford N—(N-(((9H-fluoren-9-yl)methoxy)carbonyl)-N-methylglycyl)-N-methylglycine, Int 10b, (200 g, 523 mmol, 99% yield) as yellow oil. ¹H NMR (400 MHz, DMSO-d₆) δ=7.89 (br t, J=8.8 Hz, 2H), 7.72-7.56 (m, 2H), 7.41 (q, J=7.2 Hz, 2H), 7.37-7.25 (m, 2H), 4.35-4.23 (m, 2H), 4.23-4.08 (m, 2H), 4.07-3.97 (m, 3H), 3.01-2.77 (m, 6H).

### Step C. Preparation of Int 10d

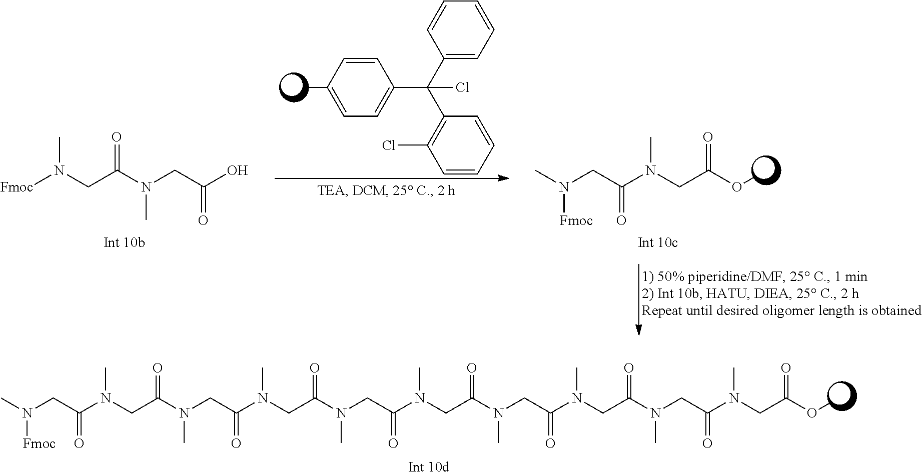

To a solution of N—(N-(((9H-fluoren-9-yl)methoxy)carbonyl)-N-methylglycyl)-N-methylglycine, Int 10b, (35.0 g, 91.5 mmol, 1.00 eq) in dichloromethane (600 mL) was added to 2-chlorotrityl chloride resin (175 g). Then diisopropylethylamine (29.6 g, 228 mmol, 39.9 mL, 2.50 eq) was added. The mixture was stirred at 25° C. for 2 h. The mixture was filtered and washed with dimethyl formamide (600 mL) and dichloromethane/methanol/diisopropylethylamine (600 mL, 80:15:5). The mixture was filtered and washed with dimethyl formamide (3×600 mL) and dichloromethane (3×600 mL). This reaction was in parallel twice. The resin, Int 10c, was stored at 0° C. for next step.

Elongation of the polysarcosine oligomer was performed until the desired length was obtained, by alternating remove Fmoc and amide coupling steps. In the first step, the resin, Int 10c, was treated with piperidine (50%) in dimethyl formamide at 25° C. for 1 min and washed with dimethyl formamide (4 times). For the amide coupling step, to the resin was added a solution of N—(N-(((9H-fluoren-9-yl)methoxy)carbonyl)-N-methylglycyl)-N-methylglycine (1.30 eq), O-(7-azabenzotriazol-1-yl)-N,N,N′,N′-tetramethyluronium hexafluorophosphate (1.50 eq) and diisopropylethylamine (2 eq) in dimethylformamide. The reaction vessel was stirred at 25° C. for 2 h and the resin was extensively washed with dimethyl formamide (4 times) and dichloromethane (3 times). The resin, Int 10d, was dried under vacuum and stored at 0° C.

### Step D. Preparation of Int 10e

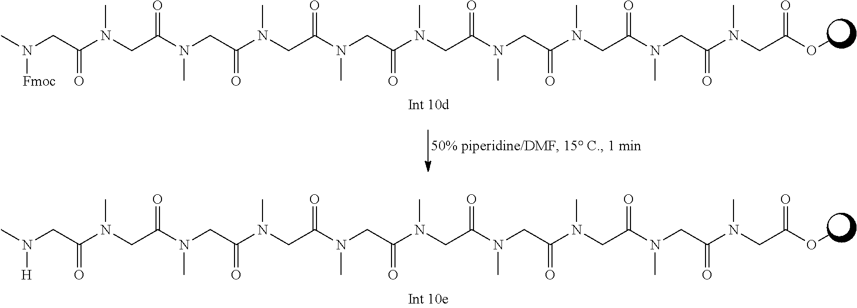

The resin, Int 10d, in piperidine (500 mL) and dimethyl formamide (500 mL) was agitated with nitrogen atmosphere at 15° C. for 1 min. The mixture was filtered and washed with dimethyl formamide (3×500 mL) and dichloromethane (500 mL), then the filter cake was concentrated in vacuum. The resin, Int 10e, (the equivalent of 50 g of Int 10f loaded on resin) was dried under vacuum and stored at 0° C.

### Step E. Preparation of Int 10f

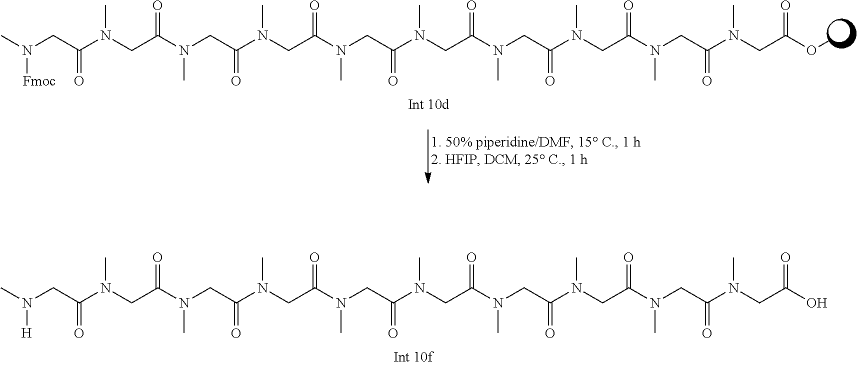

The resin, Int 10d, (42 g) in piperidine (40.0 mL) and dimethyl formamide (160 mL) was agitated with nitrogen atmosphere for 1 h at 25° C. Then the solution was drained and the resin was washed with dimethyl formamide (3×120 mL) and dichloromethane (3×120 mL). Then it was agitated in 1,1,1,3,3,3-Hexafluro-2-propanol (40 mL) and DCM (160 mL) with nitrogen atmosphere for 1 h at 25° C. and filtered. The filtrate was concentrated to give a residue, which was purified by prep-HPLC (column: Waters Atlantis T3 150*30 mm*5 um; mobile phase: [water(FA)-ACN];gradient: 1%-20% B over 10 min) to give 5,8,11,14,17,20,23,26,29-nonamethyl-4,7,10,13,16,19,22,25,28-nonaoxo-2,5,8,11,14,17,20,23,26,29-decaazahentriacontan-31-oic acid, Int 10f, (550 mg, 754 μmol) as a white solid. ¹H NMR (400 MHz, DMSO-d₆) δ=4.50-3.50 (m, 24H), 2.95-270 (m, 23H), 2.49-2.37 (m, 3H).

### Step F. Preparation of Int 10g

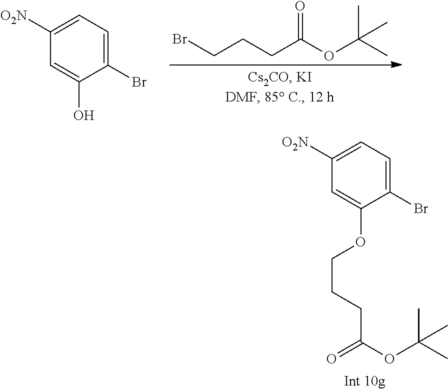 [SMILES: O=[N+]([O-])c1ccc(Br)c(O)c1 ; CC(C)(C)OC(=O)CCCBr ; CC(C)(C)OC(=O)CCCOc1cc([N+](=O)[O-])ccc1Br]

To a mixture of 2-bromo-5-nitrophenol (125 g, 573 mmol, 1.00 eq) and cesium carbonate (374 g, 1.15 μmol, 2.00 eq), potassium iodide (9.52 g, 57.3 mmol, 0.100 eq) in N,N-dimethyl formamide (500 mL) was added tert-butyl 4-bromobutanoate (141 g, 631 mmol, 1.10 eq). The mixture was stirred at 85° C. for 12 h. The reaction mixture was poured into ice water (5 L). After a large quantity of brown precipitate was formed. The precipitate was washed with water (300 mL). After filtration, the solid was concentrated under reduced pressure to afford tert-butyl 4-(2-bromo-5-nitrophenoxy)butanoate, Int 10g, (372 g, crude) as a yellow solid which was used in next step without purification. ¹H NMR (400 MHz, DMSO-d₆) δ=7.88 (d, J=8.4 Hz, 1H), 7.81 (d, J=2.0 Hz, 1H), 7.74 (dd, J=2.4, 8.8 Hz, 1H), 4.21 (t, J=6.4 Hz, 2H), 2.42 (t, J=7.2 Hz, 2H), 1.99 (quin, J=6.8 Hz, 2H), 1.39 (s, 9H)

### Step G. Preparation of Int 10h

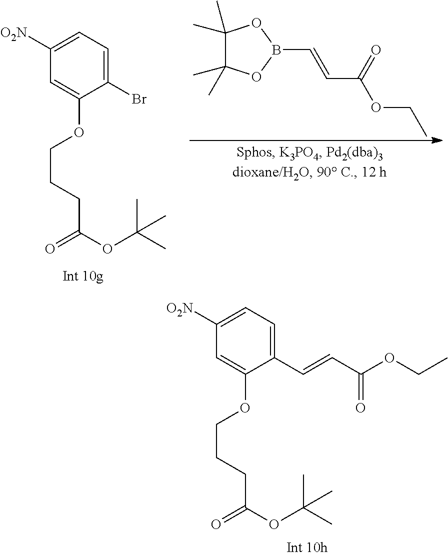 [SMILES: CC(C)(C)OC(=O)CCCOc1cc([N+](=O)[O-])ccc1Br ; CCOC(=O)/C=C/B1OC(C)(C)C(C)(C)O1 ; CCOC(=O)/C=C/c1ccc([N+](=O)[O-])cc1OCCCC(=O)OC(C)(C)C]

A mixture of tert-butyl 4-(2-bromo-5-nitrophenoxy)butanoate, Int 10g, (50.0 g, 139 mmol, 1.00 eq), ethyl (E)-3-(4,4,5,5-tetramethyl-1,3,2-dioxaborolan-2-yl)acrylate (78.5 g, 347 mmol, 2.50 eq), tris(dibenzylideneacetone) dipalladium(0) (6.36 g, 6.94 mmol, 0.0500 eq), dicyclohexyl(2′,6′-dimethoxy-[1,1′-biphenyl]-2-yl)phosphane (5.70 g, 13.9 mmol, 0.100 eq), potassium phosphate (30.0 g, 141 mmol, 1.02 eq) in water (100 mL) and dioxane (400 mL) was stirred at 90° C. for 12 h under nitrogen atmosphere. After being cooled to room temperature, the mixture was concentrated under reduced pressure to give a residue, which was purified by flash silica gel chromatography (ISCO®;330 g SepaFlash® Silica Flash Column, Eluent of 0~30% ethyl acetate/petroleum ether gradient @120 mL/min). The desired fraction collected and concentrated under reduced pressure to afford tert-butyl (E)-4-(2-(3-ethoxy-3-oxoprop-1-en-1-yl)-5-nitrophenoxy)butanoate, Int 10h, (50.0 g, 132 mmol, 94% yield) as a yellow solid. ¹H NMR (400 MHz, DMSO-d₆) δ=8.05-7.99 (m, 1H), 7.87 (d, J=16.4 Hz, 1H), 7.83 (br s, 2H), 6.82 (d, J=16.0 Hz, 1H), 4.28-4.16 (m, 4H), 2.41 (br t, J=7.2 Hz, 2H), 2.02 (quin, J=6.4 Hz, 2H), 1.27 (t, J=7.2 Hz, 3H).

### Step H. Preparation of Int 10i

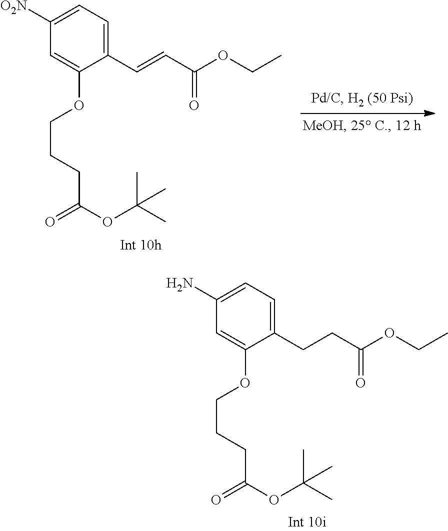 [SMILES: CCOC(=O)/C=C/c1ccc([N+](=O)[O-])cc1OCCCC(=O)OC(C)(C)C ; CCOC(=O)CCc1ccc(N)cc1OCCCC(=O)OC(C)(C)C]

To a solution of tert-butyl (E)-4-(2-(3-ethoxy-3-oxoprop-1-en-1-yl)-5-nitrophenoxy)butanoate, Int 10h, (50.0 g, 132 mmol, 1.00 eq) in methanol (500 mL) was added palladium on activated carbon (20.0 g, 10% purity) under hydrogen (50 Psi). The mixture was stirred at 25° C. for 12 h. The mixture was filtered by celite with diatomite to give the filtrate, and concentrated to afford tert-butyl 4-(5-amino-2-(3-ethoxy-3-oxopropyl)phenoxy) butanoate, Int 10i, (39.5 g, crude) as yellow oil which was used in next step without purification. ¹H NMR (400 MHz, DMSO-d₆) δ=6.74 (d, J=8.0 Hz, 1H), 6.17 (d, J=1.6 Hz, 1H), 6.05 (dd, J=1.6, 8.0 Hz, 1H), 4.89 (s, 2H), 4.04-4.00 (m, 2H), 3.86 (t, J=6.0 Hz, 2H), 2.69-2.59 (m, 2H), 2.43 (t, J=7.6 Hz, 2H), 2.39 (1, J=7.6 Hz, 2H), 1.93 (quin, J=6.8 Hz, 2H), 1.16 (t, J=7.2 Hz, 3H).

### Step I. Preparation of Int 10j

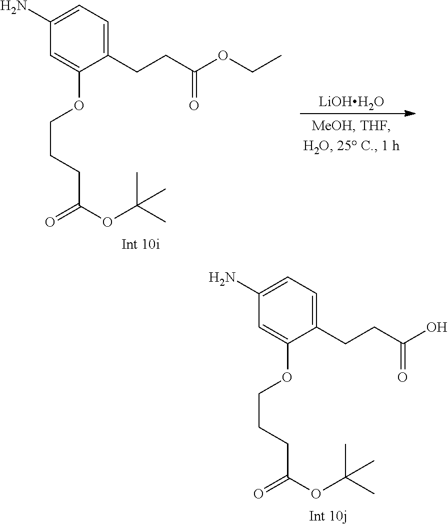 [SMILES: CCOC(=O)CCc1ccc(N)cc1OCCCC(=O)OC(C)(C)C ; "LiOH·H2O" ; CC(C)(C)OC(=O)CCCOc1cc(N)ccc1CCC(=O)O]

To a solution of tert-butyl 4-(5-amino-2-(3-ethoxy-3-oxopropyl)phenoxy)butanoate, Int 10i, (34.0 g, 96.8 mmol, 1.00 eq) in tetrahydrofuran (30.0 mL), methanol (30.0 mL) and water (30.0 mL) was added lithium hydroxide monohydrate (13.3 g, 318 mmol, 3.28 eq). The mixture was stirred at 25° C. for 1 h. The mixture was quenched with hydrochloric acid (1M) until pH=7. The mixture was concentrated to remove tetrahydrofuran and methanol. The mixture was diluted with water (300 mL) and extracted with ethyl acetate (3×150 mL). The organic layer was dried over anhydrous sodium sulfate, concentrated to afford 3-(4-amino-2-(4-(tert-butoxy)-4-oxobutoxy)phenyl)propanoic acid, Int 10j, (20.0 g, crude) as yellow oil which was used in next step without purification. ¹H NMR (400 MHz, DMSO-d₆) δ=6.73 (d, J=8.0 Hz, 1H), 6.15 (d, J=1.6 Hz, 1H), 6.03 (dd, J=1.6, 8.0 Hz, 1H), 5.13-4.51 (m, 2H), 3.85 (t, J=6.0 Hz, 2H), 2.59 (br t, J=7.6 Hz, 2H), 2.37 (t, J=7.6 Hz, 2H), 2.34-2.27 (m, 2H), 1.91 (quin, J=6.8 Hz, 2H), 1.39 (s, 9H). MS (ESI) m/z 324.0 [M+H]⁺

### Step J. Preparation of Int 10k

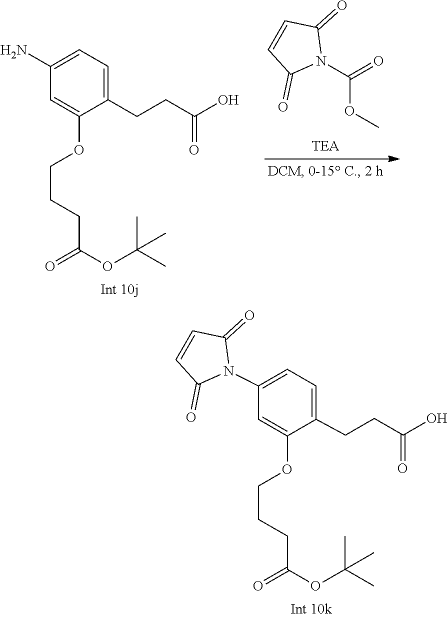 [SMILES: CC(C)(C)OC(=O)CCCOc1cc(N)ccc1CCC(=O)O ; COC(=O)N1C(=O)C=CC1=O ; CC(C)(C)OC(=O)CCCOc1cc(N2C(=O)C=CC2=O)ccc1CCC(=O)O]

To a solution of 3-(4-amino-2-(4-(tert-butoxy)-4-oxobutoxy)phenyl)propanoic acid, Int 10j, (19.5 g, 60.3 mmol, 1.00 eq) in dichloromethane (100 mL) was added triethylamine (18.3 g, 181 mmol, 25.2 mL, 3.00 eq) and methyl 2,5-dioxo-2,5-dihydro-1H-pyrrole-1-carboxylate (10.3 g, 66.3 mmol, 10.3 mL, 1.10 eq) at 0° C. The mixture was stirred at 15° C. for 2 h. The mixture was quenched with trifluoroacetic acid until pH=6, and then diluted with water (500 mL). The aqueous layer was extracted with ethyl acetate (3×300 mL). The combined organic layers were dried over anhydrous sodium sulfate, concentrated to afford 3-(2-(4-(tert-butoxy)-4-oxobutoxy)-4-(2,5-dioxo-2,5-dihydro-1H-pyrrol-1-yl)phenyl)propanoic acid, Int 10k, (33.0 g, crude) as yellow oil. MS (ESI) m/z 402.2 [M−H]⁺

### Step K. Preparation of Int 10l

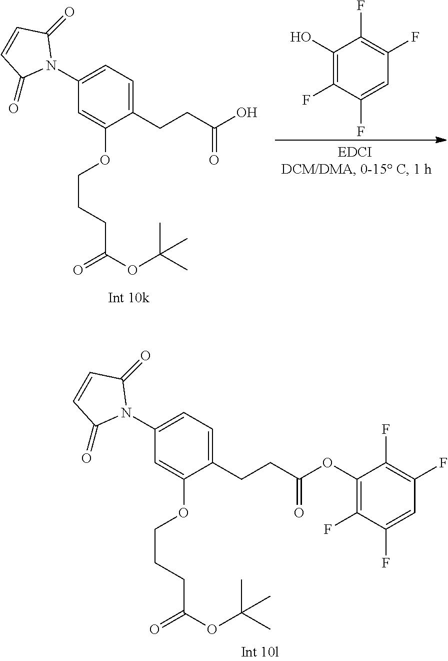 [SMILES: CC(C)(C)OC(=O)CCCOc1cc(N2C(=O)C=CC2=O)ccc1CCC(=O)O ; Oc1c(F)c(F)cc(F)c1F ; CC(C)(C)OC(=O)CCCOc1cc(N2C(=O)C=CC2=O)ccc1CCC(=O)Oc1c(F)c(F)cc(F)c1F]

To a mixture of 3-(2-(4-(tert-butoxy)-4-oxobutoxy)-4-(2,5-dioxo-2,5-dihydro-1H-pyrrol-1-yl)phenyl) propanoic acid, Int 10k, (32.0 g, 79.3 mmol, 1.00 eq) and 2,3,5,6-tetrafluorophenol (26.4 g, 159 mmol, 2.00 eq) in NN-dimethylacetamide (30.0 mL) and dichloromethane (300 mL) was added 3-(((ethylimino) methylene)amino)-N,N-dimethylpropan-1-aminium chloride, EDCI (30.4 g, 159 mmol, 2.00 eq) at 0° C., and then stirred at 15° C. for 1 h. The mixture was concentrated to give a residue, which was purified by flash silica gel chromatography (ISCO®;330 g SepaFlash® Silica Flash Column, Eluent of 0-16% ethylacetate/petroleum ether gradient @150 mL/min). The desired fraction was collected and concentrated under reduced pressure to afford tert-butyl 4-(5-(2,5-dioxo-2,5-dihydro-1H-pyrrol-1-yl)-2-(3-oxo-3-(2,3,5,6-tetrafluorophenoxy)propyl)phenoxy) butanoate, Int 10l, (29.0 g, 52.3 mmol, 66% yield) as a yellow solid. ¹H NMR (400 MHz, DMSO-d₆) δ=7.99-7.87 (m, 1H), 7.32 (d, J=8.0 Hz, 1H), 7.16 (s, 2H), 6.96 (d, J=1.2 Hz, 1H), 6.85 (dd, J=1.6, 8.0 Hz, 1H), 4.00-3.94 (m, 2H), 3.11-3.04 (m, 2H), 3.04-2.97 (m, 2H), 2.41 (t, J=7.6 Hz, 2H), 1.98-1.92 (m, 2H), 1.37 (s, 9H).

### Step L. Preparation of Int 10m

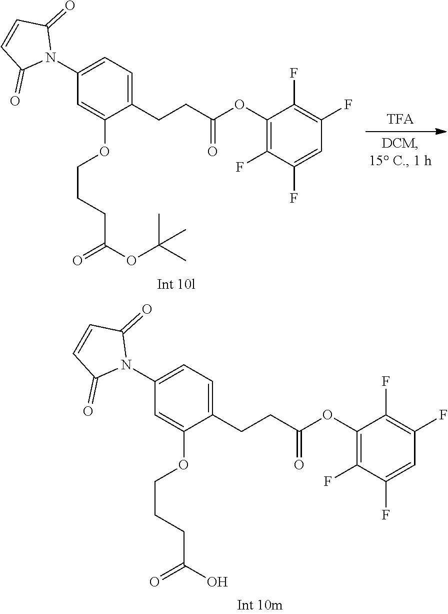 [SMILES: CC(C)(C)OC(=O)CCCOc1cc(N2C(=O)C=CC2=O)ccc1CCC(=O)Oc1c(F)c(F)cc(F)c1F ; O=C(O)CCCOc1cc(N2C(=O)C=CC2=O)ccc1CCC(=O)Oc1c(F)c(F)cc(F)c1F]

To a solution of tert-butyl 4-(5-(2,5-dioxo-2,5-dihydro-1H-pyrrol-1-yl)-2-(3-oxo-3-(2,3,5,6-tetrafluorophenoxy)propyl)phenoxy)butanoate, Int 10l, (14.5 g, 26.3 mmol, 1.00 eq) in dichloromethane (80.0 mL) was added trifluoroacetic acid (30.7 g, 269 mmol, 20.0 mL, 10.2 eq). The mixture was stirred at 15° C. for 1 h. The mixture was concentrated under reduced pressure to give a residue, which was diluted with water (300 mL) and the aqueous phase was extracted with ethyl acetate (3×200 mL). The combined organic layer was washed with brine (200 mL), dried with anhydrous sodium sulfate, filtered and concentrated in vacuum. The crude product was diluted with water (100 mL) and lyophilized, then the crude product was triturated with 2-methoxy-2-methylpropane (3×50 mL) to afford 4-(5-(2,5-dioxo-2,5-dihydro-1H-pyrrol-1-yl)-2-(3-oxo-3-(2,3,5,6-tetrafluorophenoxy)propyl)phenoxy)butanoic acid, Int 10m, (23.0 g, 46.4 mmol, 88% yield) as a white solid. ¹H NMR (400 MHz, DMSO-d₆) δ=12.11 (br s, 1H), 7.98-7.86 (m, 1H), 7.31 (d, J=8.0 Hz, 1H), 7.16 (s, 2H), 6.97 (d, J=2.0 Hz, 1H), 6.84 (dd, J=1.6, 7.6 Hz, 1H), 4.00 (t, J=6.0 Hz, 2H), 3.14-3.05 (m, 2H), 3.04-2.96 (m, 2H), 2.42 (t, J=7.6 Hz, 2H), 1.99 (quin, J=6.4 Hz, 2H)

### Step M. Preparation of Int 10n

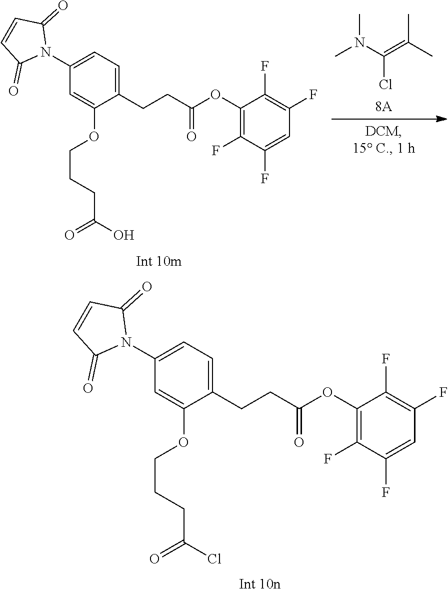

To a solution of 4-(5-(2,5-dioxo-2,5-dihydro-1H-pyrrol-1-yl)-2-(3-oxo-3-(2,3,5,6-tetrafluorophenoxy)propyl) phenoxy)butanoic acid, Int 10m, (2.00 g, 4.04 mmol, 1.00 eq) in dichloromethane (10.0 mL) was added 1-chloro-N,N,2-trimethylprop-1-en-1-amine (1.08 g, 8.07 mmol, 1.07 mL, 2.00 eq). The mixture was stirred at 15° C. for 1 h. The mixture was concentrated under reduced pressure to afford 2,3,5,6-tetrafluorophenyl 3-(2-(4-chloro-4-oxobutoxy)-4-(2,5-dioxo-2,5-dihydro-1H-pyrrol-1-yl)phenyl)propanoate, Int 10n, (2.00 g, crude) as yellow oil which was used in next step without purification.

### Step N. Preparation of Int 10o

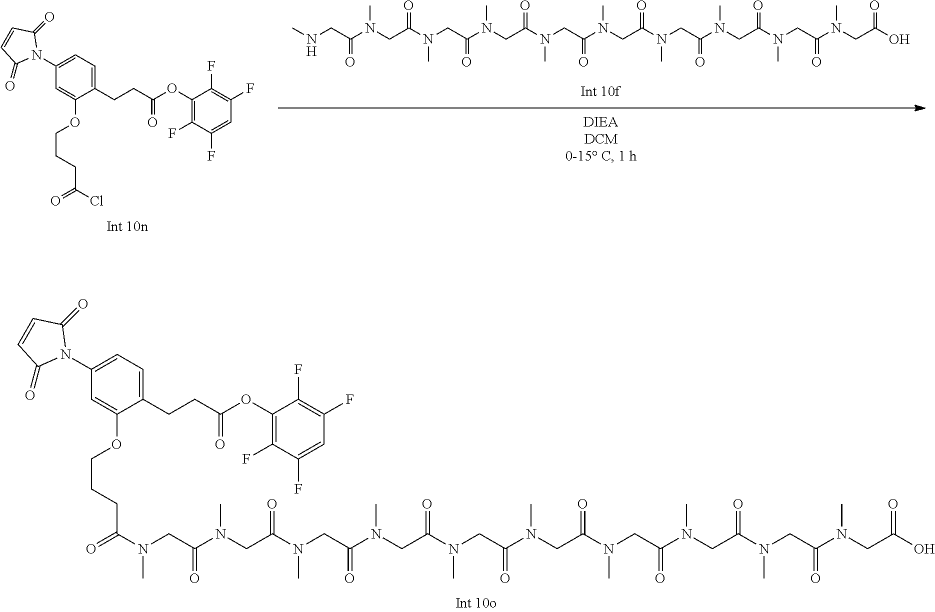 [SMILES: O=C(Cl)CCCOc1cc(N2C(=O)C=CC2=O)ccc1CCC(=O)Oc1c(F)c(F)cc(F)c1F ; CNCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O ; CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCCOc1cc(N2C(=O)C=CC2=O)ccc1CCC(=O)Oc1c(F)c(F)cc(F)c1F]

To a solution of 2,3,5,6-tetrafluorophenyl 3-(2-(4-chloro-4-oxobutoxy)-4-(2,5-dioxo-2,5-dihydro-1H-pyrrol-1-yl)phenyl)propanoate, Int 10n, (2.00 g, 3.89 mmol, 1.00 eq) in dichloromethane (20.0 mL) was added diisopropylethylamine (1.51 g, 11.7 mmol, 2.03 mL, 3.00 eq) and 5,8,11,14,17,20,23,26,29-nonamethyl-4,7,10,13,16,19,22,25,28-nonaoxo-2,5,8,11,14,17,20,23,26,29-decaazahentriacontan-31-oic acid, Int 10f, (2.84 g, 3.89 mmol, 1.00 eq) at 0° C. The mixture was stirred at 15° C. for 1 h. The mixture was concentrated under reduced pressure to give a residue, which was purified by reversed-phase HPLC (column: spherical C18, 20-45 um, 100 Å, SW 80, mobile phase: [water(0.1% Formic Acid)-ACN];B %: 10%-60%, 30 min). The desired fraction was collected and lyophilized to afford 34-(5-(2,5-dioxo-2,5-dihydro-1H-pyrrol-1-yl)-2-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, Int 10o, (1.30 g, 1.08 mmol, 27% yield) as a white solid. ¹H NMR (400 MHz, DMSO-d₆) δ=8.01-7.87 (m, 1H), 7.34-7.26 (m, 1H), 7.17 (s, 2H), 6.96 (br d, J=8.8 Hz, 1H), 6.86-6.79 (m, 1H), 4.35-3.94 (m, 22H), 2.98-2.71 (m, 34H), 2.53-2.52 (m, 1H), 2.34-2.23 (m, 1H), 2.01-1.90 (m, 2H)

### Step O. Preparation of Int 10p

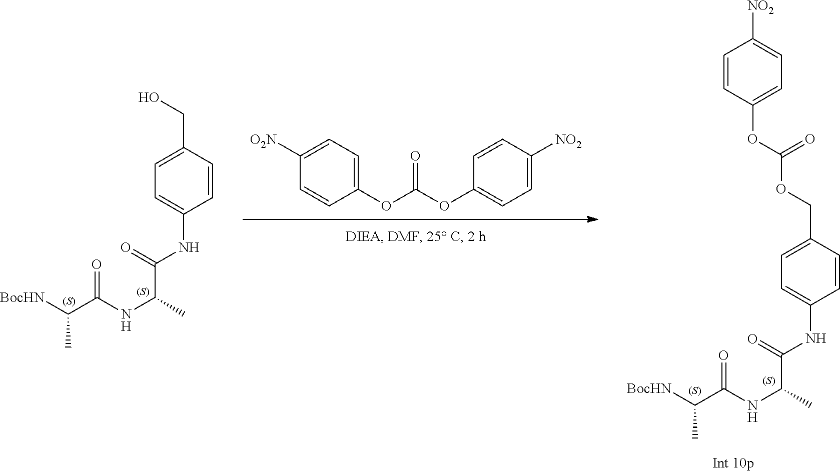

To a solution of tert-butyl ((S)-1-(((S)-1-((4-(hydroxymethyl)phenyl)amino)-1-oxopropan-2-yl)amino)-1-oxopropan-2-yl)carbamate (3.00 g, 8.21 mmol, 1.00 eq) in N,N-dimethylformamide (30.0 mL) was added bis(4-nitrophenyl) carbonate (2.50 g, 8.21 mmol, 1.00 eq) and diisopropylethylamine (1.59 g, 12.3 mmol, 2.14 mL, 1.50 eq). The mixture was stirred at 25° C. for 2 h. The mixture was diluted with water (200 mL) and extracted with ethyl acetate (3×400 mL). The combined organic layers were washed with brine (2×200 mL), dried over sodium sulfate and filtered. The filtrate was concentrated under reduced pressure to give a residue. The residue was purified by flash silica gel chromatography (ISCO®; 80 g SepaFlash® Silica Flash Column, Eluent of 0~60% ethyl acetate/petroleum ether @100 mL/min) to afford tert-butyl ((S)-1-(((S)-1-((4-((((4-nitrophenoxy)carbonyl)oxy)methyl)phenyl)amino)-1-oxopropan-2-yl)amino)-1-oxopropan-2-yl) carbamate, Int 10p, (2.70 g, 5.04 mmol, 61% yield, 99% purity) as a yellow solid. MS (ESI) m/z 531.1 [M+H]⁺

### Step P. Preparation of Int 10q

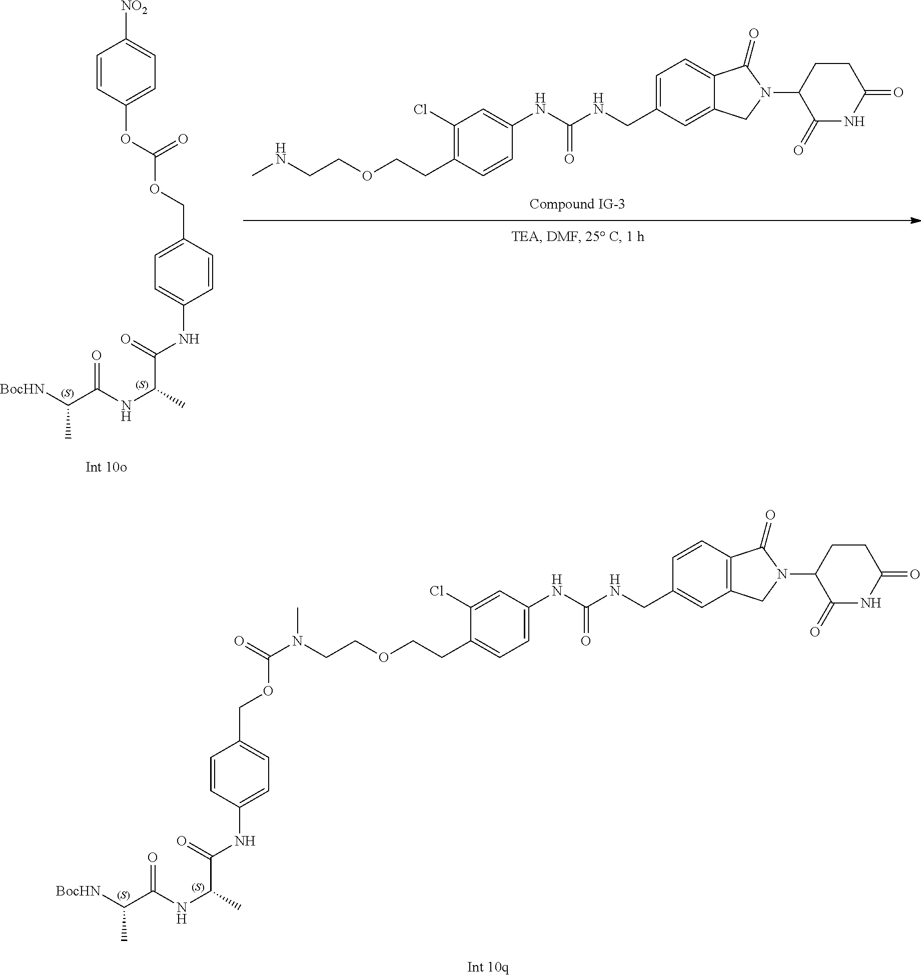

To a solution of tert-butyl ((S)-1-(((S)-1-((4-((((4-nitrophenoxy)carbonyl)oxy)methyl)phenyl)amino)-1-oxopropan-2-yl)amino)-1-oxopropan-2-yl)carbamate, Int 10p, (100 mg, 188 μmol, 1.00 eq) in NN-dimethylformamide (3.00 mL) was added 1-(3-chloro-4-(2-(2-(methylamino)ethoxy)ethyl)phenyl)-3-((2-(2,6-dioxopiperidin-3-yl)-1-oxoisoindolin-5-yl)methyl)urea, Compound IG-3, (99.5 mg, 188 μmol, 1.00 eq) and triethylamine (38.2 mg, 377 μmol, 52.5 μL, 2.00 eq). The mixture was stirred at 25° C. for 1 h. The mixture was concentrated under reduced pressure to give a residue. The residue was purified by reversed-phase HPLC (C18, 40 g; condition: water/acetonitrile=1/0-0/1, 0.1% formic acid) and lyophilized to afford 4-((S)-2-((S)-2-((tert-butoxycarbonyl)amino)propanamido)propanamido)benzyl (2-(2-chloro-4-(3-((2-(2,6-dioxopiperidin-3-yl)-1-oxoisoindolin-5-yl)methyl)ureido)phenethoxy)ethyl)(methyl)carbamate, Int 10q, (120 mg, 129 μmol, 69% yield, 99% purity) as a white solid. ¹H NMR (400 MHz, DMSO-d₆) δ=10.97 (s, 1H), 9.79 (br s, 1H), 8.79 (s, 1H), 8.21-7.96 (m, 1H), 7.71-7.65 (m, 2H), 7.63-7.56 (m, 2H), 7.51 (s, 1H), 7.44 (d, J=8.0 Hz, 1H), 7.29 (br d, J=7.2 Hz, 2H), 7.21-7.10 (m, 2H), 7.07-6.94 (m, 1H), 6.82 (t, J=6.0 Hz, 1H), 5.10 (dd, J=5.2, 13.2 Hz, 1H), 4.99 (s, 2H), 4.49-4.26 (m, 5H), 3.99 (quin, J=6.8 Hz, 1H), 3.56-3.46 (m, 4H), 3.37-3.34 (m, 2H), 2.97-2.86 (m, 1H), 2.83 (br d, J=8.4 Hz, 5H), 2.60 (br dd, J=1.6, 17.2 Hz, 1H), 2.46-2.34 (m, 1H), 2.04-1.94 (m, 1H), 1.40-1.32 (m, 9H), 1.29 (dd, J=2.4, 7.2 Hz, 3H), 1.17 (br d, J=7.2 Hz, 3H). MS (ESI) m/z 919.2 [M+H]⁺

### Step Q. Preparation of Int 10r

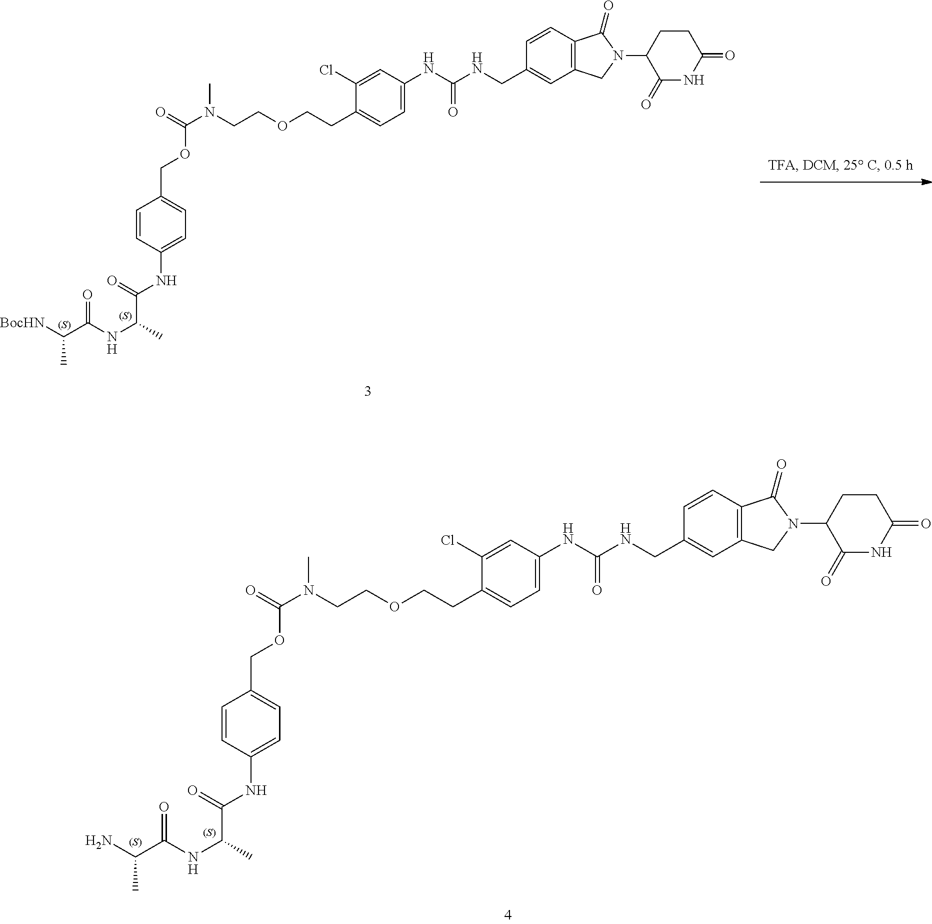

To a solution of 4-((S)-2-((S)-2-((tert-butoxycarbonyl)amino)propanamido)propanamido)benzyl (2-(2-chloro-4-(3-((2-(2,6-dioxopiperidin-3-yl)-1-oxoisoindolin-5-yl)methyl)ureido)phenethoxy)ethyl)(methyl)carbamate, Int 10q, (100 mg, 109 μmol, 1.00 eq) in dichloromethane (4.00 mL) was added trifluoroacetic acid (1.54 g, 13.5 mmol, 1.00 mL, 124 eq). The mixture was stirred at 25° C. for 0.5 h. The mixture was concentrated under reduced pressure to afford 4-((S)-2-((S)-2-aminopropanamido)propanamido)benzyl (2-(2-chloro-4-(3-((2-(2,6-dioxopiperidin-3-yl)-1-oxoisoindolin-5-yl)methyl)ureido)phenethoxy)ethyl)(methyl)carbamate, Int 10r, (80.0 mg, 68.4 mol, 63% yield, 70% purity) as a white solid. MS (ESI) m/z 819.4 [M+H]⁺

### Step R. Preparation of Compound IG-L-20

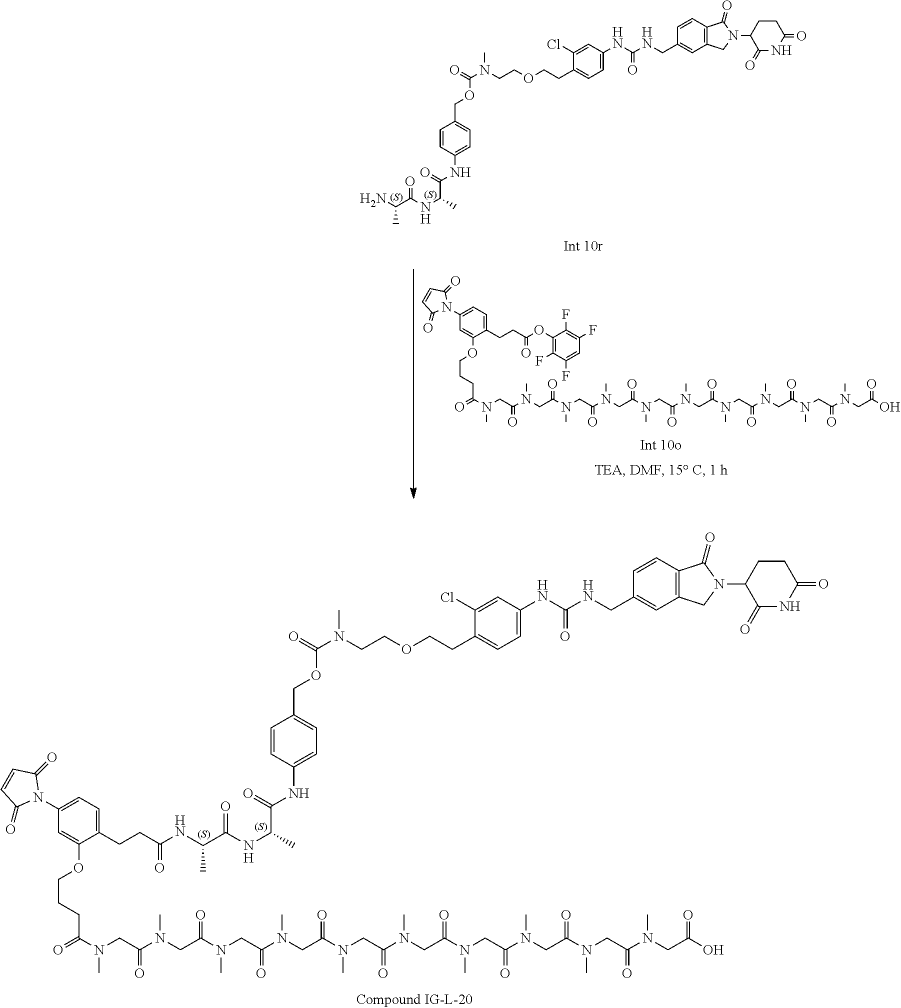

To a solution of 4-((S)-2-((S)-2-aminopropanamido)propanamido)benzyl (2-(2-chloro-4-(3-((2-(2,6-dioxopiperidin-3-yl)-1-oxoisoindolin-5-yl)methyl)ureido)phenethoxy)ethyl)(methyl)carbamate, Int 10r, (40.0 mg, 34.2 μmol, 1.00 eq) in NN-dimethylformamide (1.00 mL) was added triethylamine (6.92 mg, 68.4 μmol, 9.51 μL, 2.00 eq) and 34-(5-(2,5-dioxo-2,5-dihydro-1H-pyrrol-1-yl)-2-(3-oxo-3-(2,3,5,6-tetrafluorophenoxy) propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, Int 10o, (53.6 mg, 44.4 μmol, 1.30 eq). The mixture was stirred at 15° C. for 1 h. The residue was purified by Prep-HPLC (column: Phenomenex luna C18 150*25 mm*10 um; mobile phase: [water(FA)-ACN];gradient: 20%-50% B over 10 min), Prep-HPLC (column: Phenomenex luna C18 150*25 mm*10 um; mobile phase: [water(FA)-ACN];gradient: 20%-50% B over 10 min) and lyophilized to afford 34-(2-(3-(((2S)-1-(((2S)-1-((4-((((2-(2-chloro-4-(3-((2-(2,6-dioxopiperidin-3-yl)-1-oxoisoindolin-5-yl)methyl)ureido)phenethoxy)ethyl) (methyl)carbamoyl)oxy)methyl)phenyl)amino)-1-oxopropan-2-yl)amino)-1-oxopropan-2-yl) amino)-3-oxopropyl)-5-(2,5-dioxo-2,5-dihydro-1H-pyrrol-1-yl)phenoxy)-3,6,9,12,15,18,21,24,27,30-decamethyl-4,7,10,13,16,19,22,25,28,31-decaoxo-3,6,9,12,15,18,21, 24,27,30-decaazatetratriacontanoic acid, Compound IG-L-20, (19.3 mg, 9.87 μmol, 29% yield, 95% purity) as a white solid. ¹H NMR (400 MHz, DMSO-d₆) δ=10.96 (s, 1H), 9.92-9.71 (m, 1H), 8.81 (s, 1H), 8.45-8.27 (m, 1H), 8.22-8.02 (m, 1H), 7.72-7.58 (m, 4H), 7.51 (s, 1H), 7.43 (d, J=7.6 Hz, 1H), 7.29 (br d, J=8.4 Hz, 2H), 7.22-7.11 (m, 5H), 6.90 (br d, J=9.2 Hz, 1H), 6.79-6.73 (m, 1H), 5.10 (dd, J=4.8, 13.4 Hz, 1H), 4.98 (s, 2H), 4.47-4.18 (m, 15H), 4.09-3.88 (m, 12H), 3.55-3.45 (m, 4H), 3.36-3.33 (m, 2H), 3.00-2.70 (m, 42H), 2.62-2.57 (m, 1H), 2.40-2.36 (m, 1H), 2.01-1.89 (m, 3H), 1.30 (br d, J=7.2 Hz, 3H), 1.19 (br d, J=7.2 Hz, 3H). MS (ESI) m/z 930.8 [1/2M+H]⁺

TABLE 13
The following compounds were prepared in a manner similar to that described for Compound IG-L-20 using the
appropriate compound as starting material.

| IG-L | | |
|---|---|---|
| No. | Structure | ¹H NMR/MS |
| IG-L- 21 | 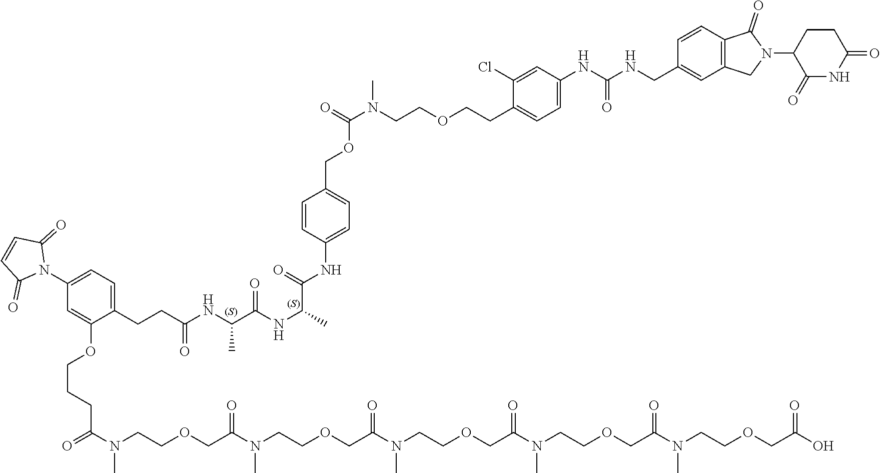 | ¹H NMR (400 MHz, DMSO-d₆) δ = 10.98 (s, 1H), 9.95- 9.76 (m, 1H), 8.79 (br s, 1H), 8.36-8.03 (m, 2H), 7.72- 7.56 (m, 4H), 7.51 (s, 1H), 7.43 (d, J = 8.0 Hz, 1H), 7.29 (br d, J = 7.2 Hz, 2H), 7.22-7.11 (m, 5H), 6.90 (br s, 1H), 6.86-6.73 (m, 2H), 5.10 (dd, J = 5.2, 13.2 Hz, 1H), 4.98 (s, 2H), 4.48-4.27 (m, 6H), 4.23-4.09 (m, 8H), 4.02-3.90 (m, 4H), 3.58-3.38 (m, 28H), 3.00- 2.89 (m, 8H), 2.85-2.77 (m, 15H), 2.61 (br d, J = 3.6 Hz, 1H), 2.45-2.35 (m, 3H), 2.04-1.88 (m, 3H), 1.30 (br d, J = 7.2 Hz, 3H), 1.19 (d, J = 7.2 Hz, 3H). MS (ESI) m/z 863.1 [1/2M + H]⁺ |
| IG-L- 22 | 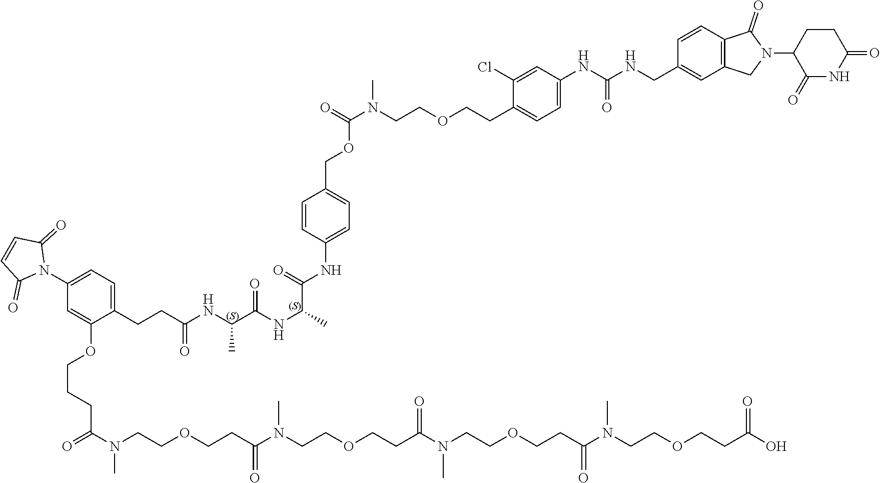 | ¹H NMR (400 MHz, DMSO-d₆) δ = 12.20 (s, 1H), 10.97 (br s, 1H), 10.04-9.75 (m, 1H), 9.20-8.87 (m, 1H), 8.43-8.06 (m, 2H), 7.70-7.59 (m, 4H), 7.51 (s, 1H), 7.43 (br d, J = 8.0 Hz, 1H), 7.28 (br d, J = 6.8 Hz, 2H), 7.24-7.10 (m, 6H), 6.90 (br s, 1H), 6.81-6.74 (m, 1H), 5.10 (br dd, J = 5.6, 13.2 Hz, 1H), 4.98 (br s, 2H), 4.49- 4.23 (m, 7H), 3.95 (br d, J = 5.6 Hz, 2H), 3.64-3.36 (m, 35H), 2.98-2.89 (m, 8H), 2.87-2.75 (m, 15H), 2.62- 2.56 (m, 2H), 2.41-2.35 (m, 3H), 2.02-1.92 (m, 3H), 1.30 (br d, J = 7.2 Hz, 3H), 1.19 (br d, J = 7.2 Hz, 3H). MS (ESI) m/z 833.6 [1/2M + H]⁺ |
| IG-L- 25 | 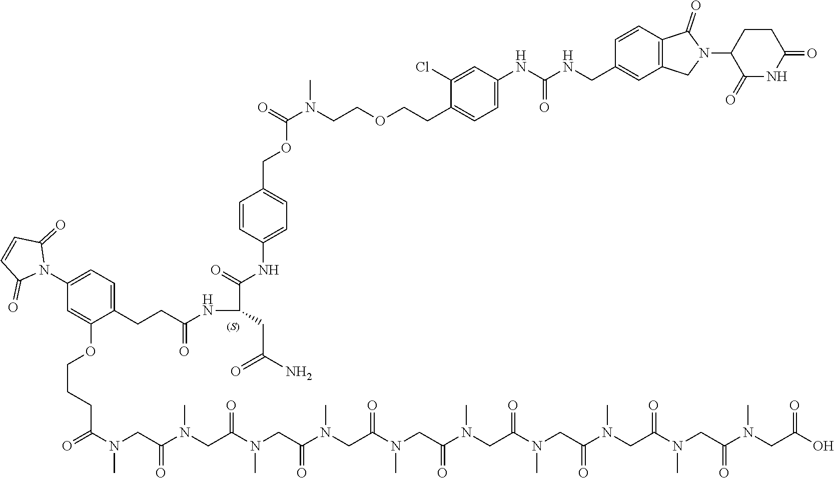 | ¹H NMR (400 MHz, DMSO-d₆) δ = 13.09-12.53 (m, 1H), 10.98 (s, 1H), 10.01 (br s, 1H), 8.78 (s, 1H), 8.20 (br d, J = 7.6 Hz, 1H), 7.72-7.65 (m, 2H), 7.59 (br d, J = 8.0 Hz, 2H), 7.51 (s, 1H), 7.44 (d, J = 8.0 Hz, 1H), 7.34-7.24 (m, 3H), 7.23-7.09 (m, 5H), 6.94-6.84 (m, 2H), 6.81 (br t, J = 6.0 Hz, 1H), 6.75 (br d, J = 8.0 Hz, 1H), 5.10 (dd, J = 13.2, 5.2 Hz, 1H), 4.98 (br s, 2H), 4.71 (q, J = 7.2 Hz, 1H), 4.48-3.88 (m, 26H), 3.57- 3.45 (m, 4H), 3.37-3.34 (m, 2H), 3.00-2.70 (m, 38H), 2.65-2.53 (m, 3H), 2.47-2.23 (m, 5H), 2.04-1.88 (m, 3H). MS (ESI) m/z 1833.2 [M + H]⁺ |
| IG-L- 57 | 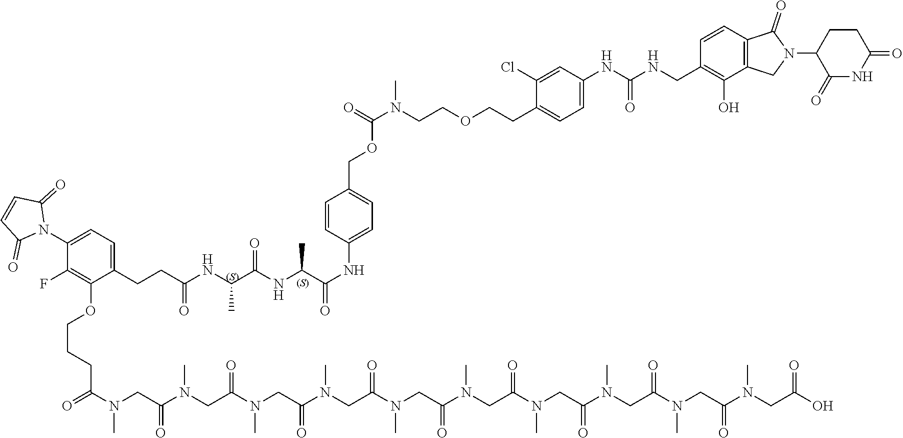 | 1H NMR (400 MHz, DMSO-d6) δ = 11.08-10.88 (m, 1H), 10.01-9.90 (m, 1H), 8.29-8.12 (m, 2H), 7.65 (br s, 1H), 7.60 (br d, J = 7.6 Hz, 2H), 7.35-7.25 (m, 4H), 7.24 (s, 2H), 7.20-7.10 (m, 4H), 7.06-6.99 (m, 1H), 5.08 (dd, J = 5.2, 13.0 Hz, 1H), 4.98 (br s, 2H), 4.39- 3.88 (m, 28H), 3.74 (br s, 1H), 3.57-3.44 (m, 5H), 2.96- 2.70 (m, 40H), 2.57 (br s, 1H), 2.48-2.43 (m, 2H), 2.37 (br dd, J = 5.2, 13.2 Hz, 1H), 2.03-1.96 (m, 1H), 1.96-1.85 (m, 2H), 1.29 (br d, J = 7.2 Hz, 3H), 1.19 (br d, J = 7.2 Hz, 3H). LC-MS (ESI) m/z 947.4[M/2 + H]⁻ |

### Example L-15: Synthesis of (2S)—N-(4-(((5-((3-(3-chloro-4-methylphenyl)ureido)methyl)-2-(2,6-dioxopiperidin-3-yl)-1-oxoisoindolin-4-yl)oxy)methyl)phenyl)-2-((S)-2-(3-(2,5-dioxo-2,5-dihydro-1H-pyrrol-1-yl)propanamido)propanamido)propenamide (IG-L-15)

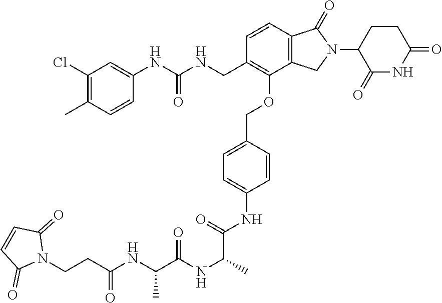 [SMILES: Cc1ccc(NC(=O)NCc2ccc3c(c2OCc2ccc(NC(=O)[C@H](C)NC(=O)[C@H](C)NC(=O)CCN4C(=O)C=CC4=O)cc2)CN(C2CCC(=O)NC2=O)C3=O)cc1Cl]

### Step A. Preparation of Int 10s

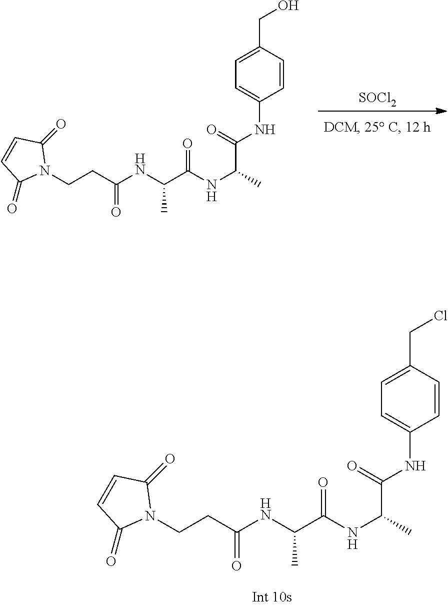 [SMILES: C[C@H](NC(=O)CCN1C(=O)C=CC1=O)C(=O)N[C@@H](C)C(=O)Nc1ccc(CO)cc1 ; "SOCl2" ; C[C@H](NC(=O)CCN1C(=O)C=CC1=O)C(=O)N[C@@H](C)C(=O)Nc1ccc(CCl)cc1]

To a solution of 3-(2,5-dioxo-2,5-dihydro-1H-pyrrol-1-yl)-N—((S)-1-(((S)-1-((4-(hydroxymethyl)phenyl) amino)-1-oxopropan-2-yl)amino)-1-oxopropan-2-yl)propanamide (3.00 g, 7.20 mmol, 1.00 eq) in dichloromethane (40.0 mL) was added thionyl chloride (1.71 g, 14.4 mmol, 1.05 mL, 2.00 eq). The mixture was stirred at 25° C. for 12 h. The mixture was concentrated under reduced pressure to give a residue. The residue was purified by triturated with ethyl acetate (30.0 mL) and filtered to afford (S)—N-(4-(chloromethyl)phenyl)-2-((S)-2-(3-(2,5-dioxo-2,5-dihydro-1H-pyrrol-1-yl) propanamido)propanamido) propenamide, Int 10s, (2.00 g, 3.22 mmol, 22% yield, 70% purity) as a yellow solid. ¹H NMR (400 MHz, DMSO-d₆) δ=9.93 (s, 1H), 8.22 (d, J=7.0 Hz, 1H), 8.13 (d, J=7.2 Hz, 1H), 7.63 (d, J=8.4 Hz, 2H), 7.36 (d, J=8.8 Hz, 2H), 7.00 (s, 2H), 4.74-4.68 (m, 3H), 4.37 (br t, J=7.2 Hz, 1H), 3.61 (t, J=7.2 Hz, 2H), 2.41 (t, J=7.2 Hz, 2H), 1.31 (d, J=7.2 Hz, 3H), 1.18 (d, J=7.2 Hz, 3H).

### Step B. Preparation of Compound IG-L-15

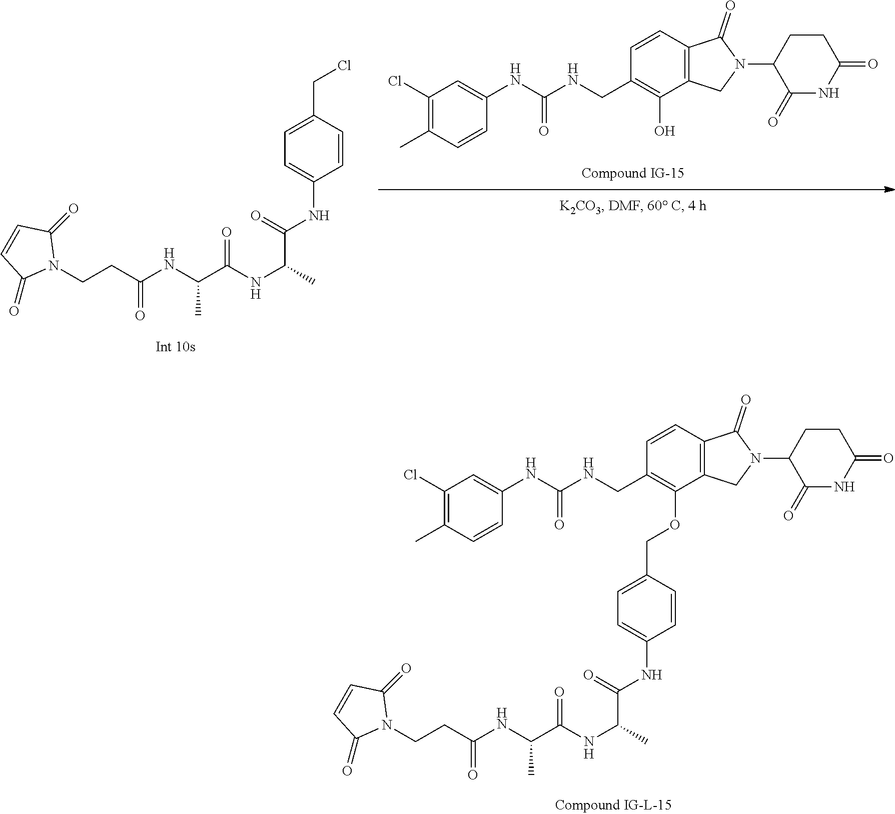 [SMILES: C[C@H](NC(=O)CCN1C(=O)C=CC1=O)C(=O)N[C@@H](C)C(=O)Nc1ccc(CCl)cc1 ; Cc1ccc(NC(=O)NCc2ccc3c(c2O)CN(C2CCC(=O)NC2=O)C3=O)cc1Cl ; "Compound IG-15" ; Cc1ccc(NC(=O)NCc2ccc3c(c2OCc2ccc(NC(=O)[C@H](C)NC(=O)[C@H](C)NC(=O)CCN4C(=O)C=CC4=O)cc2)CN(C2CCC(=O)NC2=O)C3=O)cc1Cl]

To a solution of 1-(3-chloro-4-methylphenyl)-3-((2-(2,6-dioxopiperidin-3-yl)-4-hydroxy-1-oxoisoindolin-5-yl)methyl)urea, Compound IG-15, (20.0 mg, 43.8 μmol, 1.00 eq) and (S)—N-(4-(chloromethyl)phenyl)-2-((S)-2-(3-(2,5-dioxo-2,5-dihydro-1H-pyrrol-1-yl)propanamido)propanamido)propanamide, Int 10s, (79.3 mg, 109 μmol, 2.50 eq) in N,N-dimethylformamide (1.00 mL) was added potassium carbonate (12.1 mg, 87.5 μmol, 2.00 eq). The mixture was stirred at 60° C. for 4 h under nitrogen atmosphere. The mixture was adjusted pH=4 with formic acid and filtered, the filtrate was purified by Prep-HPLC (column: Phenomenex Luna C18 150*25 mm*10 um; mobile phase: [water(TFA)-ACN]; gradient: 35%-65% B over 10 min), Prep-HPLC (column: Waters Xbridge 150*25 mm*5 um; mobile phase: [water(NH4HCO3)-ACN];gradient: 26%-56% B over 9 min), Prep-HPLC (column: Phenomenex Luna C18 150*25 mm*10 um; mobile phase: [water(TFA)-ACN];gradient: 35%-65% B over 10 min) and lyophilized to afford (2S)—N-(4-(((5-((3-(3-chloro-4-methylphenyl)ureido)methyl)-2-(2,6-dioxopiperidin-3-yl)-1-oxoisoindolin-4-yl)oxy)methyl)phenyl)-2-((S)-2-(3-(2,5-dioxo-2,5-dihydro-1H-pyrrol-1-yl)propanamido)propanamido)propenamide, Compound IG-L-15, (3.58 mg, 4.14 μmol, 2% yield, 99% purity) as a white solid. ¹H NMR (400 MHz, DMSO-d₆) δ=10.99 (s, 1H), 9.88 (s, 1H), 8.73 (s, 1H), 8.20 (d, J=6.8 Hz, 1H), 8.11 (d, J=6.8 Hz, 1H), 7.68-7.62 (m, 3H), 7.47-7.40 (m, 4H), 7.18-7.15 (m, 1H), 7.12-7.09 (m, 1H), 6.99 (s, 2H), 6.62 (br t, J=5.6 Hz, 1H), 5.20-5.06 (m, 3H), 4.64-4.55 (m, 1H), 4.54-4.46 (m, 1H), 4.42-4.35 (m, 3H), 4.23 (t, J=7.2 Hz, 1H), 3.61 (s, 2H), 2.92 (br s, 1H), 2.65-2.57 (m, 1H), 2.40 (br t, J=7.2 Hz, 2H), 2.33 (s, 1H), 2.22 (s, 3H), 1.98 (br s, 1H), 1.31 (d, J=7.2 Hz, 3H), 1.18 (d, J=7.2 Hz, 3H).

The following compounds were prepared in a manner similar to that described for Isoindolinone-glutarimide linker compound IG-L-15 of Table 2 using the appropriate compound as starting material.

TABLE 14

| IG-L No. | Structure | ¹H NMR/MS |
|---|---|---|
| | 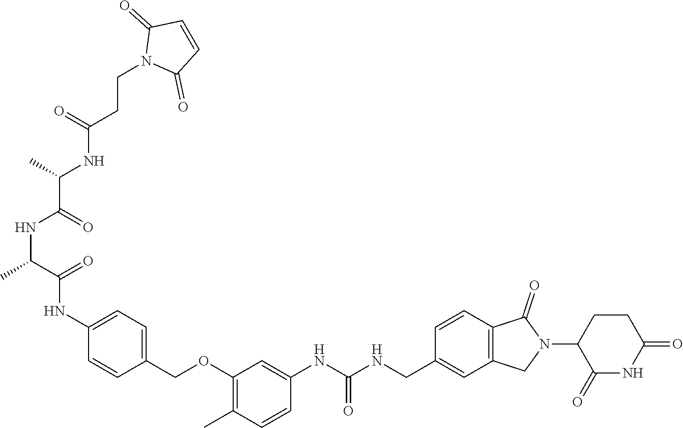 [SMILES: Cc1ccc(NC(=O)NCc2ccc3c(c2)CN(C2CCC(=O)NC2=O)C3=O)cc1OCc1ccc(NC(=O)[C@H](C)NC(=O)[C@H](C)NC(=O)CCN2C(=O)C=CC2=O)cc1] | 855.3 |
| IG-L-32 | 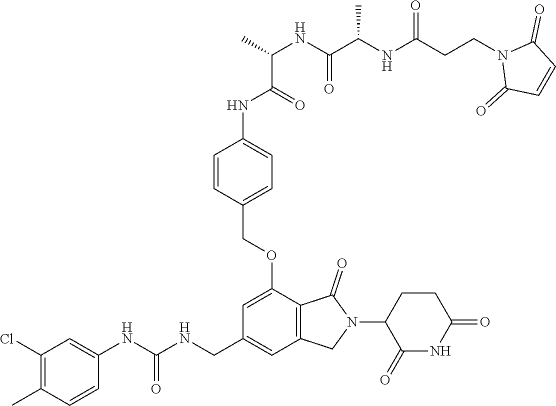 [SMILES: Cc1ccc(NC(=O)NCc2cc3c(c(OCc4ccc(NC(=O)[C@H](C)NC(=O)[C@H](C)NC(=O)CCN5C(=O)C=CC5=O)cc4)c2)C(=O)N(C2CCC(=O)NC2=O)C3)cc1Cl] | 855.3 |
| IG-L-33 | 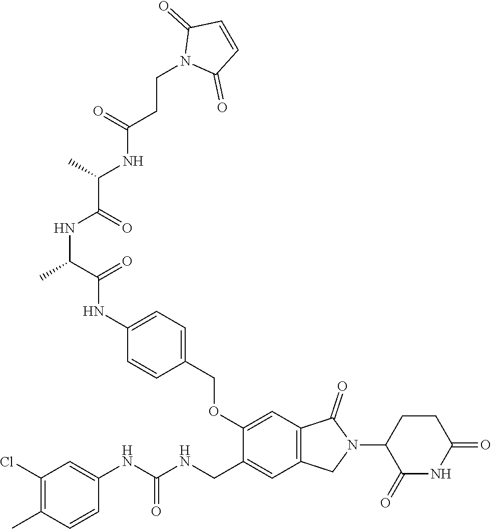 [SMILES: Cc1ccc(NC(=O)NCc2cc3c(cc2OCc2ccc(NC(=O)[C@H](C)NC(=O)[C@H](C)NC(=O)CCN4C(=O)C=CC4=O)cc2)C(=O)N(C2CCC(=O)NC2=O)C3)cc1Cl] | 855.3 |
| IG-L-23 | 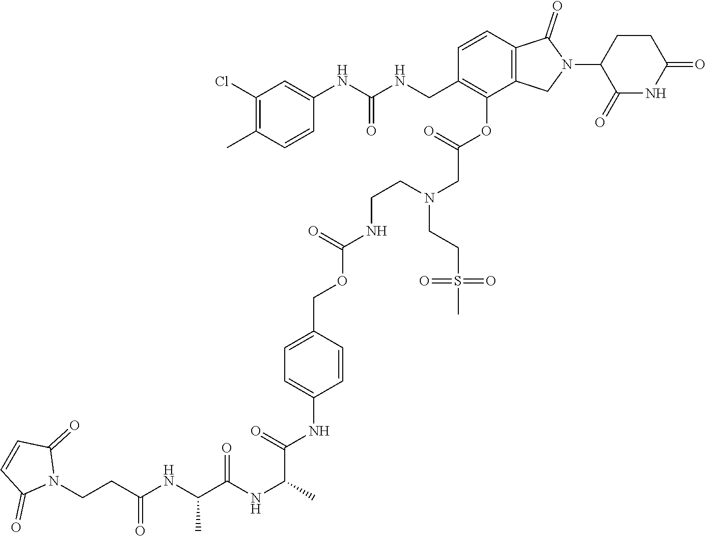 [SMILES: Cc1ccc(NC(=O)NCc2ccc3c(c2OC(=O)CN(CCNC(=O)OCc2ccc(NC(=O)[C@H](C)NC(=O)[C@H](C)NC(=O)CCN4C(=O)C=CC4=O)cc2)CCS(C)(=O)=O)CN(C2CCC(=O)NC2=O)C3=O)cc1Cl] | 1105.6 |
| IG-L-8 | 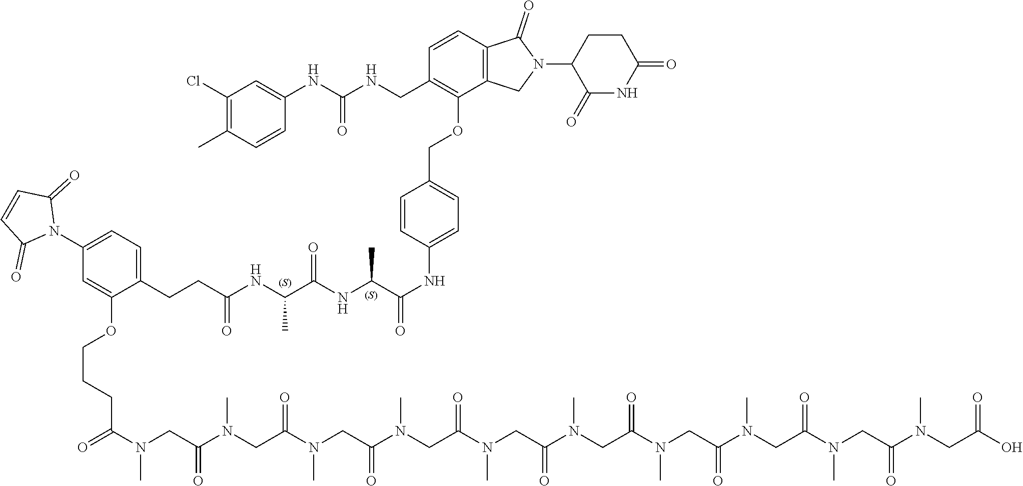 | ¹H NMR (400 MHz, DMSO-d₆) δ = 11.00 (br s, 1H), 9.96 (br d, J = 4.3 Hz, 1H), 8.87 (br s, 1H), 8.22-8.00 (m, 2H), 7.70-7.63 (m, 3H), 7.48-7.42 (m, 4H), 7.22- 7.10 (m, 5H), 6.90 (br d, J = 8.8 Hz, 1H), 6.78 (br dd, J = 1.6, 7.6 Hz, 1H), 5.19- 5.08 (m, 3H), 4.65-4.46 (m, 2H), 4.42-4.16 (m, 14H), 4.12-3.87 (m, 13H), 3.02-2.69 (m, 36H), 2.63- 2.58 (m, 1H), 2.45-2.42 (m, 1H), 2.22 (s, 3H), 2.03- 1.89 (m, 3H), 1.31 (d, J = 7.2 Hz, 3H), 1.20 (br d, J = 7.2 Hz, 3H). MS (ESI) m/z 1742.7 [M + 2H]⁺ |
| IG-L-59 | 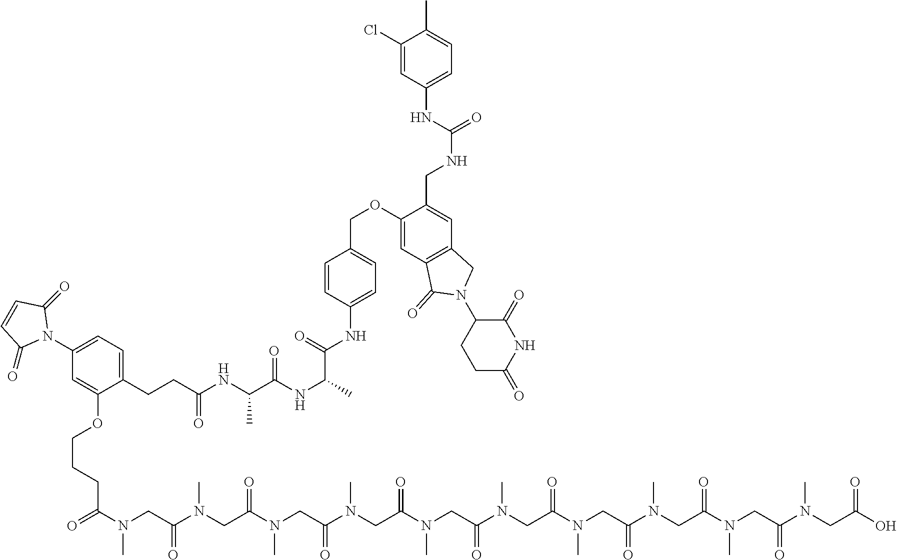 [SMILES: Cc1ccc(NC(=O)NCc2cc3c(cc2OCc2ccc(NC(=O)[C@H](C)NC(=O)[C@H](C)NC(=O)CCc4ccc(N5C(=O)C=CC5=O)cc4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc2)C(=O)N(C2CCC(=O)NC2=O)C3)cc1Cl] | ¹H NMR (400 MHz, DMSO-d₆) δ = 10.95 (br s, 1H), 10.11-9.88 (m, 1H), 8.26- 8.02 (m, 2H), 7.71-7.62 (m, 3H), 7.45 (br d, J = 5.6 Hz, 3H), 7.32 (br d, J = 3.2 Hz, 1H), 7.23-7.15 (m, 2H), 7.14 (s, 3H), 6.89 (br d, J = 9.6 Hz, 1H), 6.82-6.73 (m, 1H), 5.22 (br s, 2H), 5.08 (dd, J = 5.2, 13.1 Hz, 1H), 4.42-4.13 (m, 16H), 4.11-3.87 (m, 12H), 3.82- 3.72 (m, 1H), 3.00-2.67 (m, 36H), 2.61-2.56 (m, 1H), 2.43 (br d, J = 6.4 Hz, 1H), 2.22 (s, 3H), 2.01-1.89 (m, 3H), 1.31 (br d, J = 7.2 Hz, 3H), 1.19 (br d, J = 7.2 Hz, 3H). MS (ESI) m/z 872.8 [M/2 + H]⁺ |

### Example L-26: Synthesis of (2S)—N-(4-(((5-((3-(3-chloro-4-methylphenyl)ureido)methyl)-2-(2,6-dioxopiperidin-3-yl)-1-oxoisoindolin-4-yl)oxy)methyl)phenyl)-2-((S)-2-(3-(2,5-dioxo-2,5-dihydro-1H-pyrrol-1-yl)propanamido)propanamido)propenamide (IG-L-26)

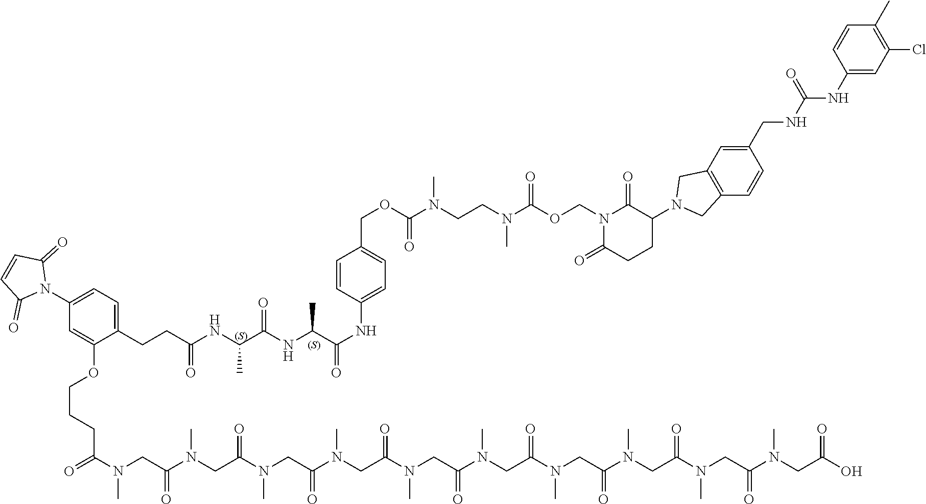

### Step A. Preparation of Int 11a

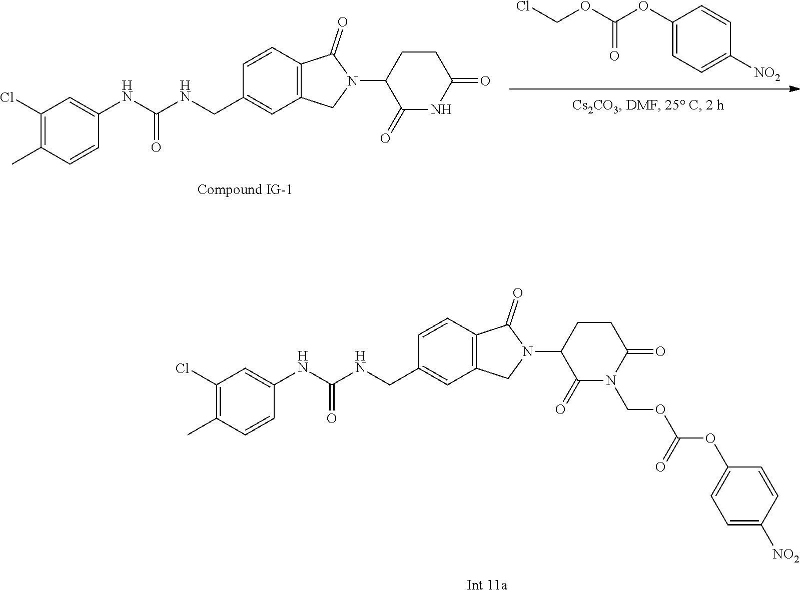 [SMILES: Cc1ccc(NC(=O)NCc2ccc3c(c2)CN(C2CCC(=O)NC2=O)C3=O)cc1Cl ; O=C(OCCl)Oc1ccc([N+](=O)[O-])cc1 ; "Compound IG-1" ; Cc1ccc(NC(=O)NCc2ccc3c(c2)CN(C2CCC(=O)N(COC(=O)Oc4ccc([N+](=O)[O-])cc4)C2=O)C3=O)cc1Cl ; "Int 11a"]

To a solution of 1-(3-chloro-4-methylphenyl)-3-((2-(2,6-dioxopiperidin-3-yl)-1-oxoisoindolin-5-yl)methyl)urea, Compound IG-1, (200 mg, 454 μmol, 1.00 eq) in dimethyl formamide (4.00 mL) was added cesium carbonate (222 mg, 681 μmol, 1.50 eq) and chloromethyl (4-nitrophenyl) carbonate (315 mg, 1.36 mmol, 3.00 eq). The mixture was stirred at 25° C. for 2 h. The mixture was filtered to give filtrate. The filtrate was purified by reversed-phase HPLC (0.1% FA condition). The desired fraction was collected and lyophilized to give was residue. The residue was purified by column chromatography (SiO2, Petroleum ether/Ethyl acetate=1/1 to 0/1). The desired fraction was collected and concentrated to give (3-(5-((3-(3-chloro-4-methylphenyl)ureido)methyl)-1-oxoisoindolin-2-yl)-2,6-dioxopiperidin-1-yl)methyl (4-nitrophenyl) carbonate, Int 11a, (250 mg, 334 μmol, 74% yield, 85% purity) as a white solid. ¹H NMR (400 MHz, DMSO-d₆) δ=8.75 (s, 1H), 8.34-8.30 (m, 1H), 7.72 (d, J=8.0 Hz, 1H), 7.66 (d, J=2.0 Hz, 1H), 7.59-7.53 (m, 2H), 7.45 (br d, J=8.0 Hz, 1H), 7.24-7.02 (m, 2H), 6.86-6.71 (m, 1H), 5.90-5.80 (m, 1H), 5.52-5.17 (m, 2H), 4.56-4.19 (m, 4H), 3.21-3.05 (m, 1H), 2.94-2.79 (m, 1H), 2.48-2.34 (m, 1H), 2.23 (s, 3H), 2.15-2.03 (m, 1H). LC-MS (ESI) m/z 636.1 [M+H]⁺

### Step B. Preparation of Int 11b

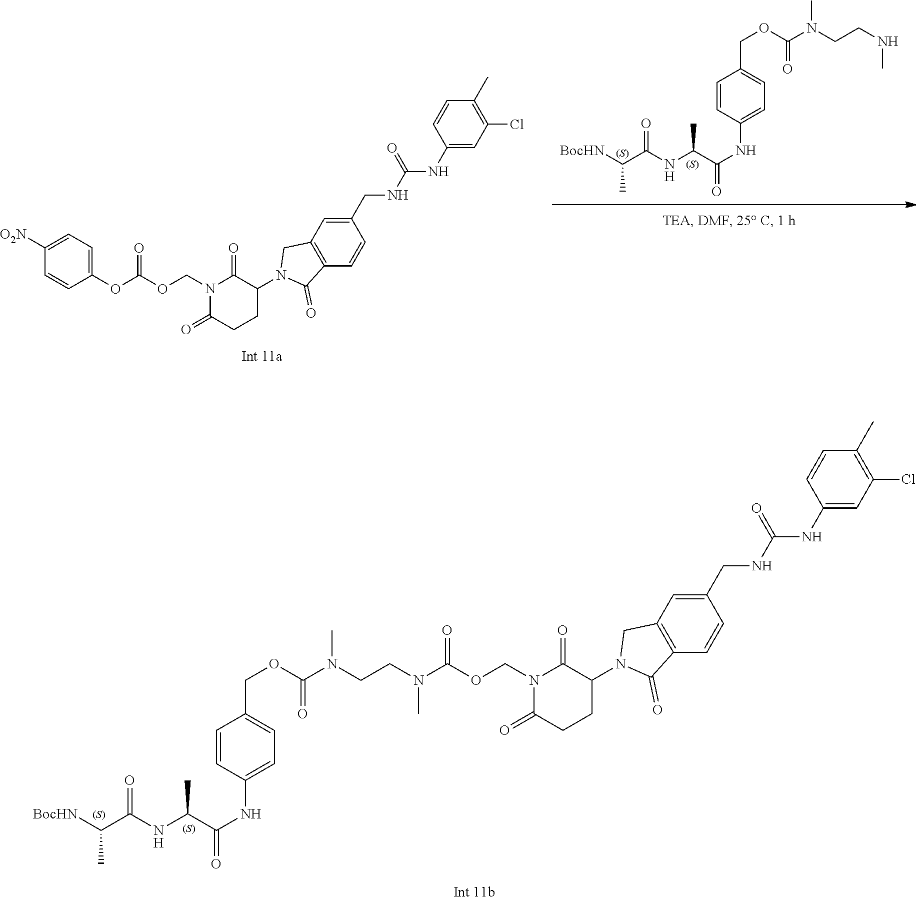

To a solution of (3-(5-((3-(3-chloro-4-methylphenyl)ureido)methyl)-1-oxoisoindolin-2-yl)-2,6-dioxopiperidin-1-yl)methyl (4-nitrophenyl) carbonate, Int 11a, (190 mg, 299 μmol, 1.00 eq) in dimethyl formamide (2.00 mL) was added triethylamine (90.7 mg, 896 μmol, 125 μL, 3.00 eq) and 4-((S)-2-((S)-2-((tert-butoxycarbonyl)amino)propanamido)propanamido)benzyl methyl(2-(methylamino)ethyl)carbamate (143 mg, 299 μmol, 1.00 eq). The mixture was stirred at 25° C. for 1 h. The mixture was filtered to give filtrate. The filtrate was purified by reversed-phase HPLC (0.1%/formic acid condition). The desired fraction was collected and lyophilized to give 4-((S)-2-((S)-2-((tert-butoxy carbonyl)amino)propanamido)propanamido) benzyl((3-(5-((3-(3-chloro-4-methylphenyl)ureido)methyl)-1-oxoisoindolin-2-yl)-2,6-dioxopiperidin-1-yl) methyl) ethane-1,2-diylbis(methylcarbamate), Int 11b, (130 mg, 113 μmol, 38% yield, 85% purity) as a white solid. ¹H NMR (400 MHz, DMSO-d₆) δ=9.98 (br s, 1H), 8.84 (s, 1H), 8.25-8.17 (m, 1H), 801 (br d, J=7.2 Hz, 1H), 7.70 (d, J=8.0 Hz, 1H), 7.57 (br d, J=8.8 Hz, 2H), 7.44 (br d, J=8.0 Hz, 1H), 7.31-7.24 (m, 3H), 7.19-7.15 (m, 1H), 7.08-6.96 (m, 1H), 6.93-6.84 (m, 1H), 5.89-5.48 (m, 2H), 5.36-5.14 (m, 1H), 5.03-4.87 (m, 2H), 4.52-4.24 (m, 4H), 4.04-3.95 (m, 1H), 3.52-3.34 (m, 3H), 3.31-3.24 (m, 2H), 3.14-3.00 (m, 1H), 2.92-2.66 (m, 8H), 2.45-2.34 (m, 1H), 2.22 (s, 2H), 2.07-1.99 (m, 2H), 1.38-1.34 (m, 9H), 1.29 (br dd, J=2.4, 6.8 Hz, 3H), 1.17 (br d, J=6.8 Hz, 3H). LC-MS (ESI) m/z 976.1 [M+H]⁺

### Step C. Preparation of Int 11c

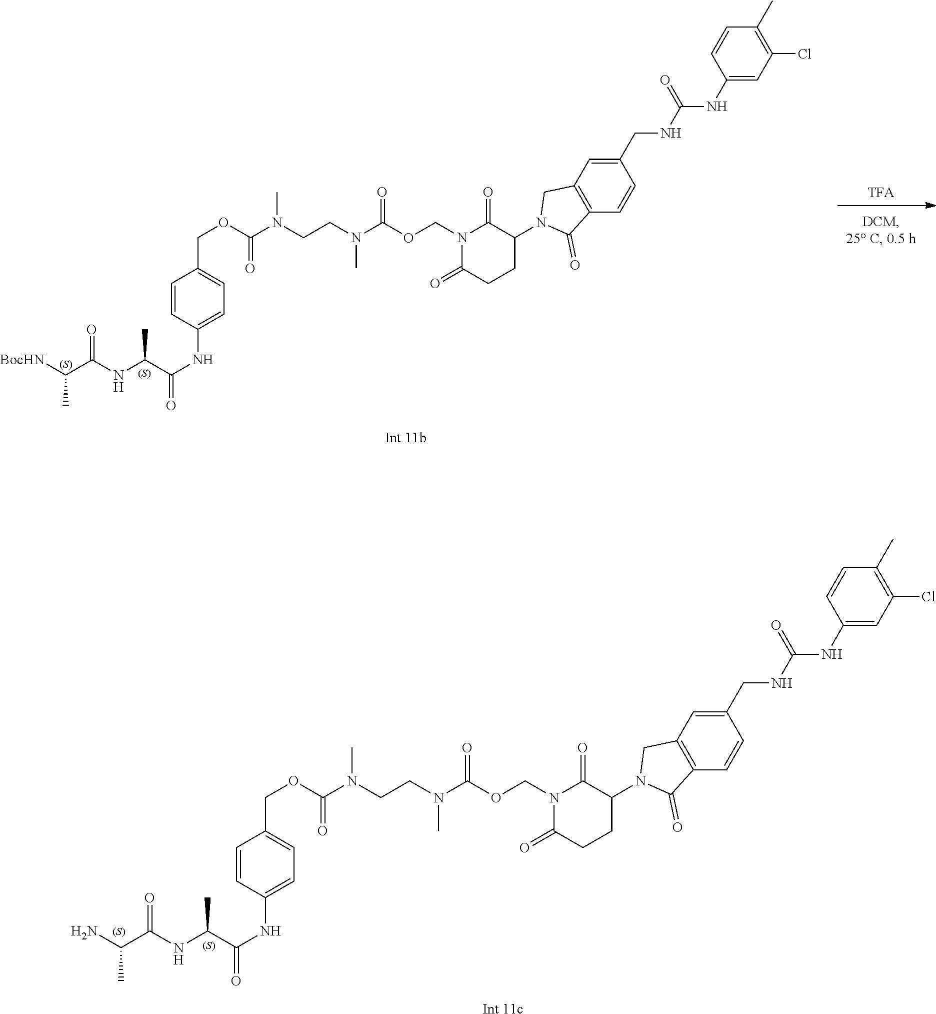

To a solution of 4-((S)-2-((S)-2-((tert-butoxycarbonyl)amino)propanamido)propanamido)benzyl ((3-(5-((3-(3-chloro-4-methylphenyl)ureido)methyl)-1-oxoisoindolin-2-yl)-2,6-dioxopiperidin-1-yl)methyl) ethane-1,2-diylbis(methylcarbamate), Int 11b, (80.0 mg, 82.0 μmol, 1.00 eq) in dichloromethane (1.00 mL) was added trifluoroacetic acid (9.34 mg, 82.0 μmol, 6.09 μL, 1.00 eq). The mixture was stirred at 25° C. for 0.5 h. The mixture was concentrated to give a residue. The residue was purified by prep-HPLC (column: Phenomenex luna C18 150*25 mm*10 um; mobile phase: [water (FA)-ACN]; gradient: 22%-52% B over 9 min). The desired fraction was collected and lyophilized to give 4-((S)-2-((S)-2-aminopropanamido)propanamido)benzyl ((3-(5-((3-(3-chloro-4-methylphenyl)ureido)methyl)-1-oxoisoindolin-2-yl)-2,6-dioxopiperidin-1-yl)methyl) ethane-1,2-diylbis(methylcarbamate), Int 11c, (30.0 mg, 32.5 μmol, 40% yield, 95% purity) as a white solid. LC-MS (ESI) m/z 876.7 [M+H]⁺

### Step D. Preparation of Compound IG-L-26

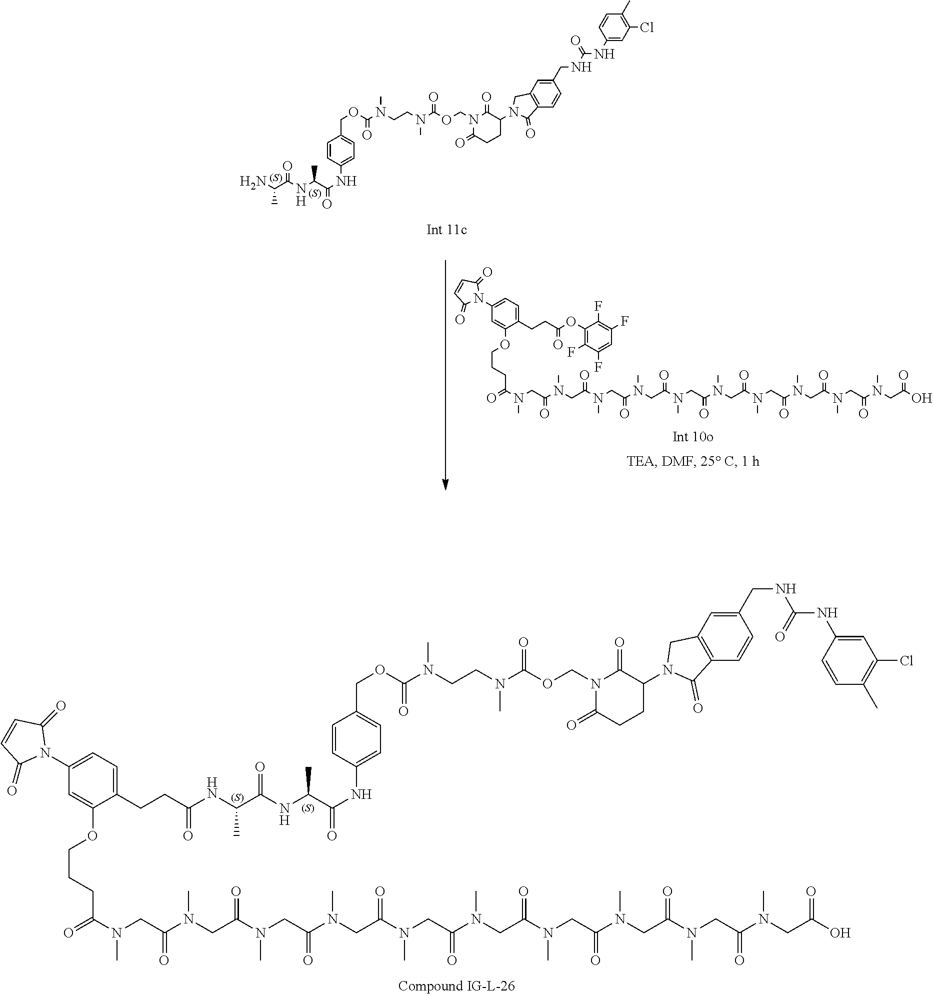

To a solution of 4-((S)-2-((S)-2-aminopropanamido)propanamido)benzyl ((3-(5-((3-(3-chloro-4-methylphenyl)ureido)methyl)-1-oxoisoindolin-2-yl)-2,6-dioxopiperidin-1-yl)methyl) ethane-1,2-diylbis (methylcarbamate), Int 11c, (20.0 mg, 22.8 μmol, 1.00 eq) in dimethyl formamide (1.00 mL) was added triethylamine (6.93 mg, 68.5 μmol, 9.53 μL, 3.00 eq) and 34-(5-(2,5-dioxo-2,5-dihydro-1H-pyrrol-1-yl)-2-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, Int 10o, (33.0 mg, 27.4 μmol, 1.20 eq). The mixture was stirred at 25° C. for 1 h. The mixture was filtered to give filtrate. The filtrate was purified by prep-HPLC (column: Phenomenex luna C18 150*25 mm*10 um; mobile phase: [water (FA)-ACN]; gradient: 35%-65% B over 9 min). The desired fraction was collected and lyophilized to give (2S)—N-(4-(((5-((3-(3-chloro-4-methylphenyl)ureido)methyl)-2-(2,6-dioxopiperidin-3-yl)-1-oxoisoindolin-4-yl)oxy)methyl)phenyl)-2-((S)-2-(3-(2,5-dioxo-2,5-dihydro-1H-pyrrol-1-yl)propanamido)propanamido)propenamide, Compound IG-L-26, (21.78 mg, 11.3 μmol, 49% yield, 99% purity) as a white solid. ¹H NMR (400 MHz, DMSO-d₆) δ=9.90-9.69 (m, 1H), 8.70 (br t, J=6.0 Hz, 1H), 8.39-7.92 (m, 2H), 7.65-7.50 (m, 4H), 7.46-7.34 (m, 2H), 7.21 (br d, J=6.8 Hz, 2H), 7.14-7.04 (m, 5H), 6.93-6.64 (m, 3H), 5.63-5.41 (m, 2H), 5.27-5.10 (m, 1H), 5.00-4.78 (m, 2H), 4.39-4.11 (m, 17H), 4.06-3.82 (m, 14H), 2.91-2.62 (m, 44H), 2.37-2.33 (m, 2H), 2.16 (s, 3H), 2.00-1.85 (m, 3H), 1.23 (br d, J=6.4 Hz, 3H), 1.12 (br d, J=6.8 Hz, 3H). LC-MS (ESI) m/z 1916.6[M+H]⁺

The compound in Table 15 was prepared in a manner similar to that described for Compound IG-L-26 using the appropriate compound as starting material.

TABLE 15

| IG- | | |
|---|---|---|
| L | | |
| No. | Structure | ¹H NMR/MS |
| IG- L- 24 | 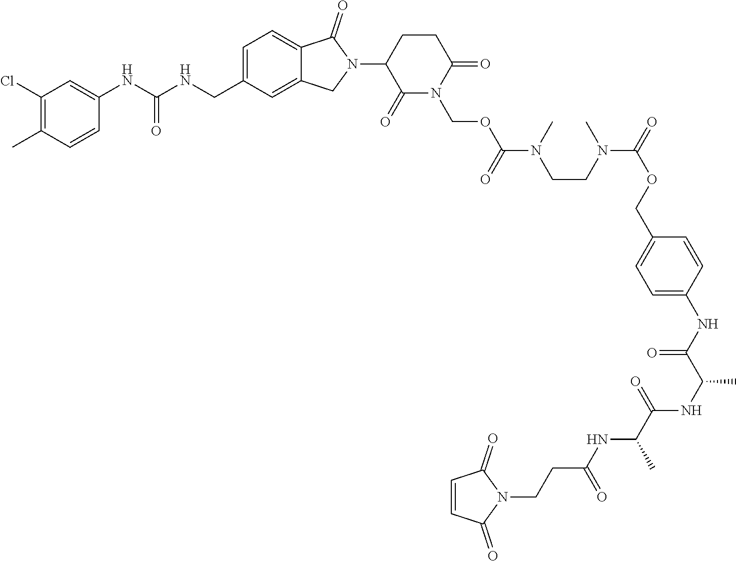 [SMILES: Cc1ccc(NC(=O)NCc2ccc3c(c2)CN(C2CCC(=O)N(COC(=O)N(C)CCN(C)C(=O)OCc4ccc(NC(=O)[C@H](C)NC(=O)[C@H](C)NC(=O)CCN5C(=O)C=CC5=O)cc4)C2=O)C3=O)cc1Cl] | ¹H NMR (400 MHz, DMSO-d₆) δ = 9.85 (s, 1H), 8.75 (s, 1H), 8.21 (d, J = 7.0 Hz, 1H), 8.12 (br d, J = 7.0 Hz, 1H), 7.70 (d, J = 7.8 Hz, 1H), 7.66 (d, J = 1.8 Hz, 1H), 7.60 (br d, J = 7.4 Hz, 2H), 7.54-7.47 (m, 1H), 7.44 (br d, J = 7.8 Hz, 1H), 7.28 (br d, J = 7.6 Hz, 2H), 7.21- 7.10 (m, 2H), 7.00 (s, 2H), 6.79 (br t, J = 5.8 Hz., 1H), 5.64-5.17 (m, 3H), 5.09-4.84 (m, 2H), 4.51- 4.19 (m, 6H), 3.61 (t, J = 7.4 Hz, 2H), 3.33- 3.21 (m, 4H), 3.15-2.98 (m, 1H), 2.86- 2.67 (m, 7H), 2.40 (br t, J = 7.2 Hz, 3H), 2.23 (s, 3H), 2.11- |
| | | 2.00 (m, 1H), |
| | | 1.31 (br d, J = |
| | | 7.0 Hz, 3H), |
| | | 1.18 (d, J = 7.0 |
| | | Hz, 3H). |
| | | LC-MS (ESI) m/z |
| | | 1027.5[M + H]⁺ |

### Example L-16: Synthesis of 1-(3-chloro-4-(2-(2-(2,5-dioxo-2,5-dihydro-1H-pyrrol-1-yl)ethoxy)ethyl)phenyl)-3-((2-(2,6-dioxopiperidin-3-yl)-1-oxoisoindolin-5-yl)methyl) urea (IG-L-16)

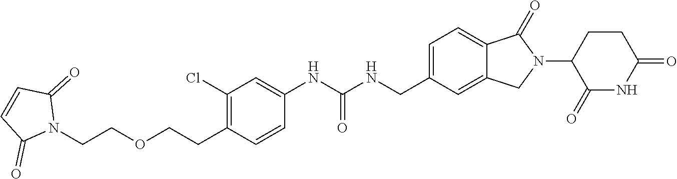 [SMILES: O=C1CCC(N2Cc3cc(CNC(=O)Nc4ccc(CCOCCN5C(=O)C=CC5=O)c(Cl)c4)ccc3C2=O)C(=O)N1]

### Step A. Preparation of Compound IG-L-16

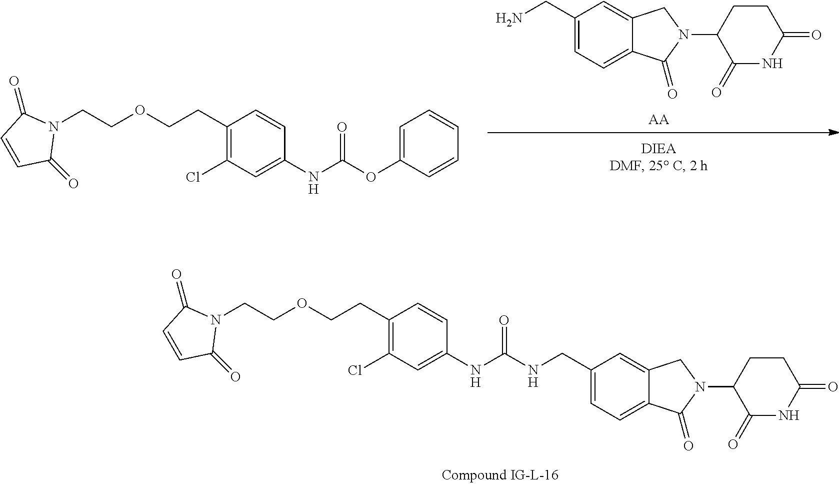

To a solution of phenyl (3-chloro-4-(2-(2-(2,5-dioxo-2,5-dihydro-1H-pyrrol-1-yl)ethoxy)ethyl)phenyl) carbamate (10.0 mg, 24.1 μmol, 1.00 eq) and 3-(5-(aminomethyl)-1-oxoisoindolin-2-yl)piperidine-2,6-dione, Int AA, (7.91 mg, 28.9 μmol, 1.20 eq) in dimethyl formamide (1.00 mL) was added N,N-diisopropylethylamine (6.23 mg, 48.2 μmol, 8.40 μL, 2.00 eq), the mixture was stirred at 25° C. for 2 h. The reaction mixture was purified by Prep-HPLC (column: Phenomenex Luna C18 150*25 mm*10 um; mobile phase: [water(formic acid)-acetonitrile];gradient: 23%-53% B over 10 min) and lyophilized to afford 1-(3-chloro-4-(2-(2-(2,5-dioxo-2,5-dihydro-1H-pyrrol-1-yl)ethoxy)ethyl)phenyl)-3-((2-(2,6-dioxopiperidin-3-yl)-1-oxoisoindolin-5-yl)methyl) urea IG-L-16 (2.38 mg, 3.81 μmol, 15% yield) as a white solid. ¹H NMR (400 MHz, DMSO-d₆) δ=11.13-10.78 (m, 1H), 8.82 (s, 1H), 7.69 (d, J=8.0 Hz, 1H), 7.64 (s, 1H), 7.51 (s, 1H), 7.44 (d, J=9.0 Hz, 1H), 7.12 (d, J=1.0 Hz, 2H), 6.99 (s, 2H), 6.89-6.83 (m, 1H), 5.15-5.05 (m, 1H), 4.48-4.39 (m, 3H), 4.34-4.28 (m, 1H), 3.55-3.49 (m, 6H), 2.93-2.87 (m, 1H), 2.76 (br t, J=7.0 Hz, 2H), 2.61 (br d, J=1.8 Hz, 1H), 2.47-2.35 (m, 1H), 2.05-1.95 (m, 1H). MS (ESI) m/z 594.3 [M+H]⁺

### Example L-18: Synthesis of 4-((37R,40S,43S)-37-(2-(2-(2-(3-(2,5-dioxo-2,5-dihydro-1H-pyrrol-1-yl)propanamido)ethoxy) ethoxy)acetamido)-40,43-dimethyl-38,41-dioxo-2,5,8,11,14,17,20,23,26,29,32-undecaoxa-35-thia-39,42-diazatetratetracontan-44-amido)benzyl (2-(2-chloro-4-(3-((2-(2,6-dioxopiperidin-3-yl)-1-oxoisoindolin-5-yl) methyl)ureido)phenethoxy)ethyl)(methyl) carbamate (IG-L-18)

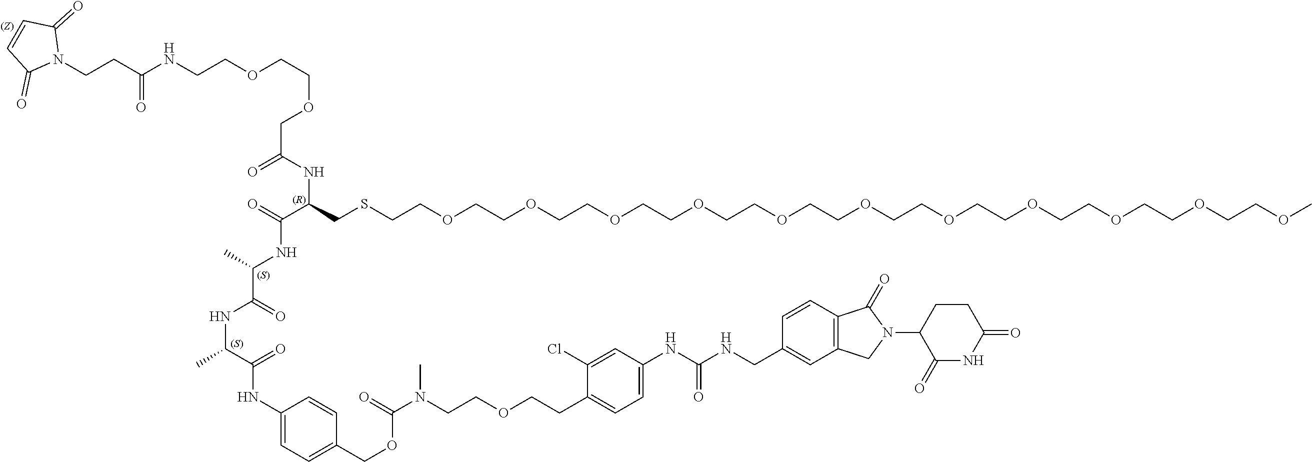

### Step A. Preparation of Int 12a

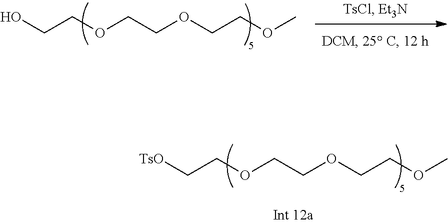

To a solution of 2,5,8,11,14,17,20,23,26,29,32-undecaoxatetratriacontan-34-ol (900 mg, 1.74 mmol, 1.00 eq) and triethylamine (353 mg, 3.48 mmol, 485 μL, 2.00 eq) in dichloromethane (10.0 mL) was added para-toluenesulfonyl chloride (664 mg, 3.48 mmol, 2.00 eq). Then the mixture was stirred at 25° C. for 12 h. The reaction mixture was concentrated to give a residue. The residue was purified by column chromatography (SiO2, Petroleum ether/Ethyl acetate=1/1 to 0/1) to afford 2,5,8,11,14,17,20,23,26-nonaoxaoctacosan-28-yl 4-methylbenzenesulfonate, Int 12a, (1.10 g, 1.64 mmol, 94% yield) as colorless oil. ¹H NMR (400 MHz, CDCl₃) δ=7.85-7.76 (m, 2H), 7.35 (d, J=8.0 Hz, 2H), 4.20-4.14 (m, 2H), 3.69-3.61 (m, 33H), 3.58 (s, 3H), 3.57-3.53 (m, 2H), 3.38 (s, 3H), 3.15-3.10 (m, 4H), 2.45 (s, 3H).

### Step B. Preparation of Int 12b

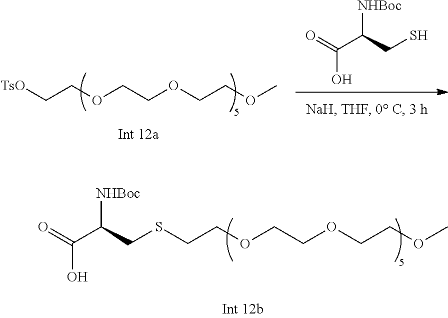

To a solution of 2,5,8,11,14,17,20,23,26-nonaoxaoctacosan-28-yl 4-methylbenzenesulfonate, Int 12a, (600 mg, 894 gmol, 1.00 eq) in tetrahydrofuran (5.00 mL) was added sodium hydride (143 mg, 3.58 mmol, 60% purity, 4.00 eq) and (tert-butoxycarbonyl)-L-cysteine (297 mg, 1.34 mmol, 1.50 eq). The mixture was stirred at 0° C. for 3 h. The mixture was concentrated under reduced pressure to give a residue. The residue was purified by reversed-phase HPLC (C18, 80 g; condition: water/acetonitrile=1/0-0/1, 0.1% formic acid) and lyophilized to afford (R)-37-((tert-butoxycarbonyl)amino)-2,5,8,11,14,17,20,23,26,29,32-undecaoxa-35-thiaoctatriacontan-38-oic acid, Int 12b, (500 mg, 695 μmol, 77% yield) as colorless oil. ¹H NMR (400 MHz, CDCl₃) δ=5.60 (br d, J=7.2 Hz, 1H), 4.53 (br d, J=6.4 Hz, 1H), 3.69-3.64 (m, 40H), 3.57-3.54 (m, 2H), 3.39 (s, 3H), 3.10 (br d, J=4.4 Hz, 2H), 2.84-2.68 (m, 2H), 1.45 (s, 9H).

### Step C. Preparation of Int 12c

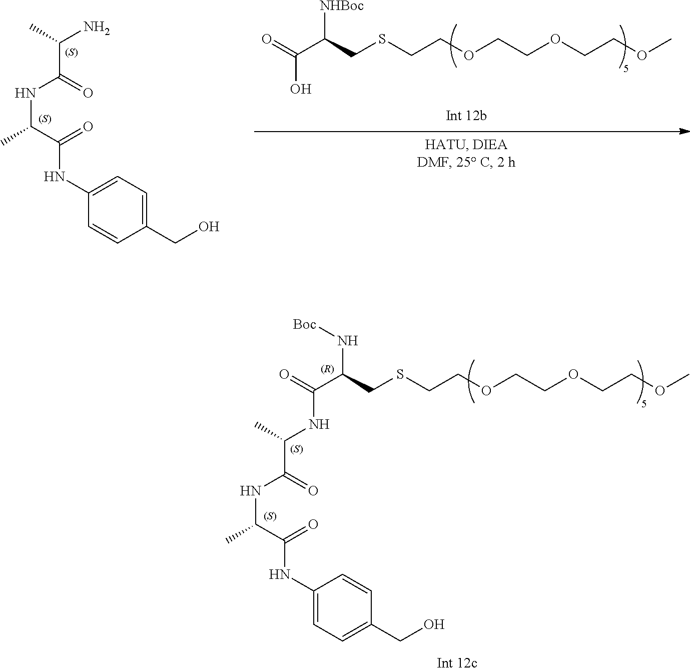

To a solution of(R)-37-((tert-butoxycarbonyl)amino)-2,5,8,11,14,17,20,23,26,29,32-undecaoxa-35-thiaoctatriacontan-38-oic acid, Int 12b, (500 mg, 695 μmol, 1.00 eq) and (S)-2-amino-N—((S)-1-((4-(hydroxymethyl)phenyl)amino)-1-oxopropan-2-yl)propanamide (184 mg, 695 μmol, 1.00 eq) in dimethyl formamide (5.00 mL) was added NN-diisopropylethylaamine (179.54 mg, 1.39 mmol, 242 μL, 2.00 eq) and O-(7-Azabenzotriazol-1-yl)-N,N,N′,N′-tetramethyluronium Hexafluorophosphate (343 mg, 902 μmol, 1.30 eq). Then the mixture was stirred at 25° C. for 2 h. The mixture was concentrated under reduced pressure to give a residue. The residue was purified by reversed-phase HPLC (C18, 80 g; condition: water/acetonitrile=1/0-0/1, 0.1% formic acid) and lyophilized to afford tert-butyl ((37R,40S,43S)-44-((4-(hydroxymethyl)phenyl) amino)-40,43-dimethyl-38,41,44-trioxo-2,5,8,11,14,17,20,23,26,29,32-undecaoxa-35-thia-39,42-diazatetratetracontan-37-yl)carbamate, Int 12c, (670 mg, 693 μmol, 99% yield) as colorless oil. ¹H NMR (400 MHz, CDCl₃) δ=8.62 (s, 1H), 7.76 (d, J=8.4 Hz, 2H), 7.61-7.46 (m, 2H), 7.31 (d, J=8.4 Hz, 2H), 5.71 (br d, J=6.8 Hz, 1H), 4.65-4.61 (m, 3H), 4.45 (q, J=6.8 Hz, 1H), 4.31 (br dd, J=4.8, 7.2 Hz, 1H), 3.67-3.60 (m, 40H), 3.56-3.54 (m, 2H), 3.38 (s, 3H), 2.92-2.87 (m, 2H), 2.84 (br d, J=4.8 Hz, 1H), 2.86-2.74 (m, 1H), 1.50 (dd, J=2.4, 7.3 Hz, 6H), 1.45 (s, 9H).

### Step D. Preparation of Int 12d

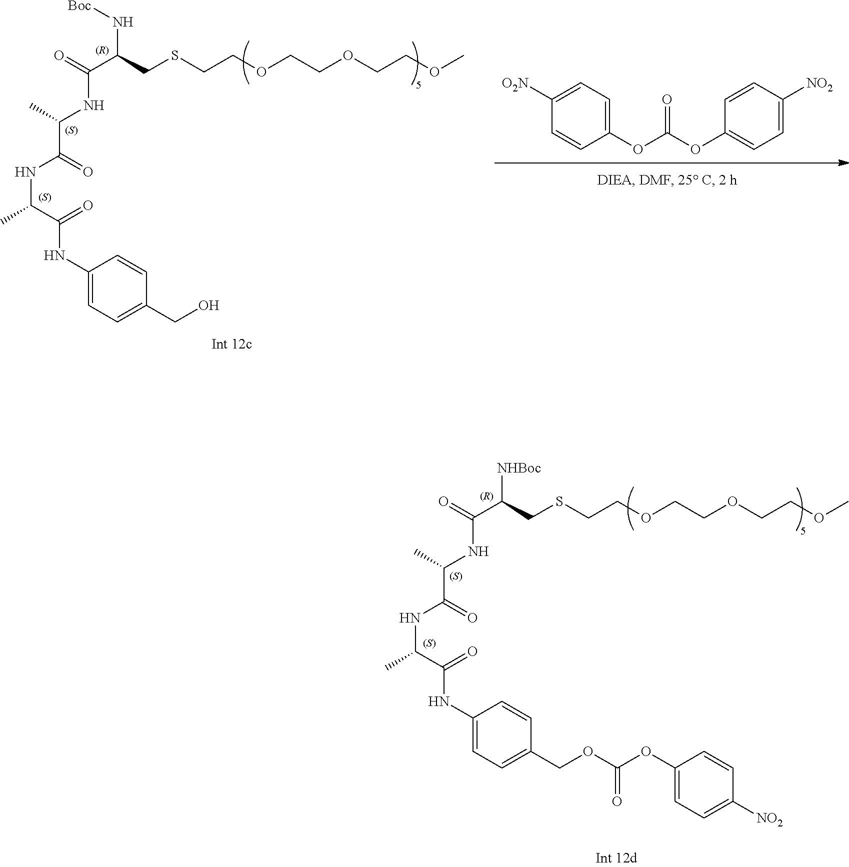

To a solution of tert-butyl ((37R,40S,43S)-44-((4-(hydroxymethyl)phenyl)amino)-40,43-dimethyl-38,41,44-trioxo-2,5,8,11,14,17,20,23,26,29,32-undecaoxa-35-thia-39,42-diazatetratetracontan-37-yl)carbamate, Int 12c, (670 mg, 693 μmol, 1.00 eq) in dimethyl formamide (3.00 mL) was added N,N-diisopropylethylamine (134 mg, 1.04 mmol, 181 IL, 1.50 eq) and bis(4-nitrophenyl) carbonate (422 mg, 1.39 mmol, 2.00 eq). The mixture was stirred at 25° C. for 2 h. The mixture was concentrated under reduced pressure to give a residue. The residue was purified by reversed-phase HPLC (C18, 80 g; condition: water/acetonitrile=1/0-0/1, 0.1% formic acid) and lyophilized to afford tert-butyl ((37R,40S,43S)-40,43-dimethyl-44-((4-((((4-nitrophenoxy)carbonyl) oxy)methyl)phenyl)amino)-38,41,44-trioxo-2,5,8,11,14,17,20,23,26,29,32-undecaoxa-35-thia-39,42-diazatetratetracontan-37-yl)carbamate, Int 12d, (700 mg, 618 μmol, 89% yield) as colorless oil. ¹H NMR (400 MHz, CDCl₃) δ=8.69 (s, 1H), 8.27 (d, J=9.2 Hz, 2H), 7.83 (br d, J=8.4 Hz, 2H), 7.65-7.48 (m, 2H), 7.38 (dd, J=2.8, 8.9 Hz, 4H), 5.71 (br d, J=6.0 Hz, 1H), 5.25 (s, 2H), 4.62 (quin, J=7.2 Hz, 1H), 4.45 (q, J=6.8 Hz, 1H), 4.35-4.27 (m, 1H), 3.68-3.60 (m, 40H), 3.57-3.54 (m, 2H), 3.38 (s, 3H), 2.91 (br d, J=6.8 Hz, 2H), 2.88-2.74 (m, 2H), 1.51 (dd, J=1.6, 7.3 Hz, 6H), 1.46 (s, 9H).

### Step E. Preparation of Int 12e

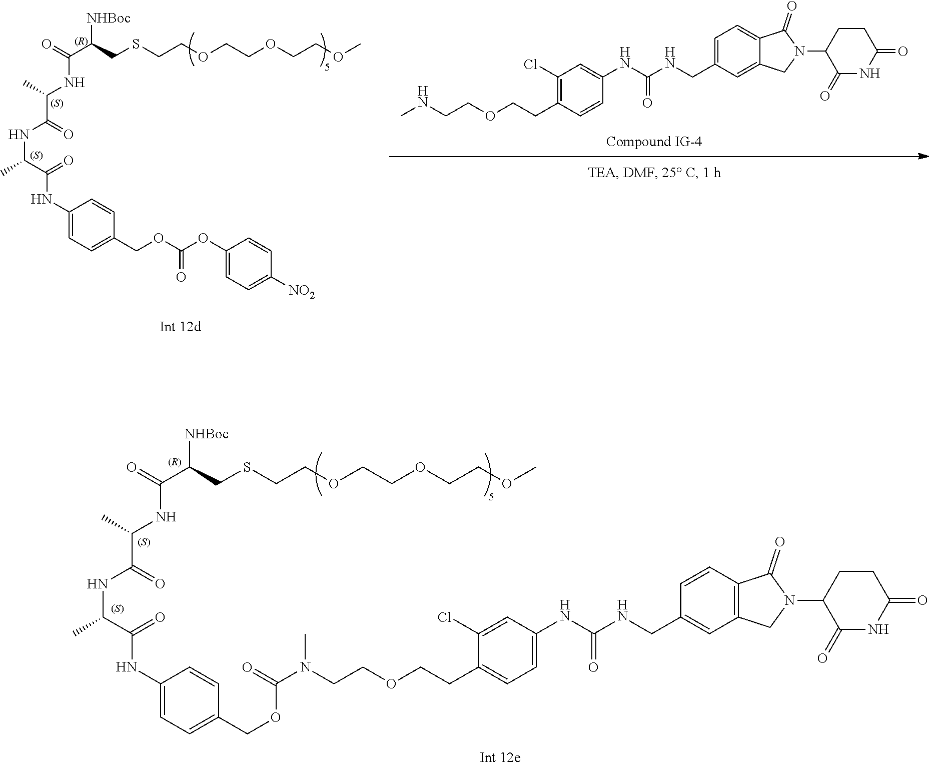

To a solution of tert-butyl ((37R,40S,43S)-40,43-dimethyl-44-((4-((((4-nitrophenoxy)carbonyl)oxy)methyl) phenyl)amino)-38,41,44-trioxo-2,5,8,11,14,17,20,23,26,29,32-undecaoxa-35-thia-39,42-diazatetratetracontan-37-yl)carbamate, Int 12d, (470 mg, 415 μmol, 1.00 eq) in dimethyl formamide (3.00 mL) was added triethylamine (84.0 mg, 830 μmol, 116 μL, 2.00 eq) and 1-(3-chloro-4-(2-(2-(methylamino)ethoxy) ethyl)phenyl)-3-((2-(2,6-dioxopiperidin-3-yl)-1-oxoisoindolin-5-yl)methyl)urea, Compound IG-4. The mixture was stirred at 25° C. for 1 h. The mixture was concentrated under reduced pressure to give a residue. The residue was purified by reversed-phase HPLC (C18, 40 g; condition: water/acetonitrile=1/0-0/1, 0.1% formic acid) and lyophilized to afford 4-((37R,40S,43S)-37-((tert-butoxycarbonyl)amino)-40,43-dimethyl-38,41-dioxo-2,5,8,11,14,17,20,23,26,29,32-undecaoxa-35-thia-39,42-diazatetratetracontan-44-amido)benzyl (2-(2-chloro-4-(3-((2-(2,6-dioxopiperidin-3-yl)-1-oxoisoindolin-5-yl)methyl)ureido)phenethoxy)ethyl)(methyl)carbamate, Int 12e, (600 mg, 390 μmol, 94% yield, 99% purity) as colorless oil. MS (ESI) m/z 1521.5 [M+H]⁺

### Step F. Preparation of Int 12

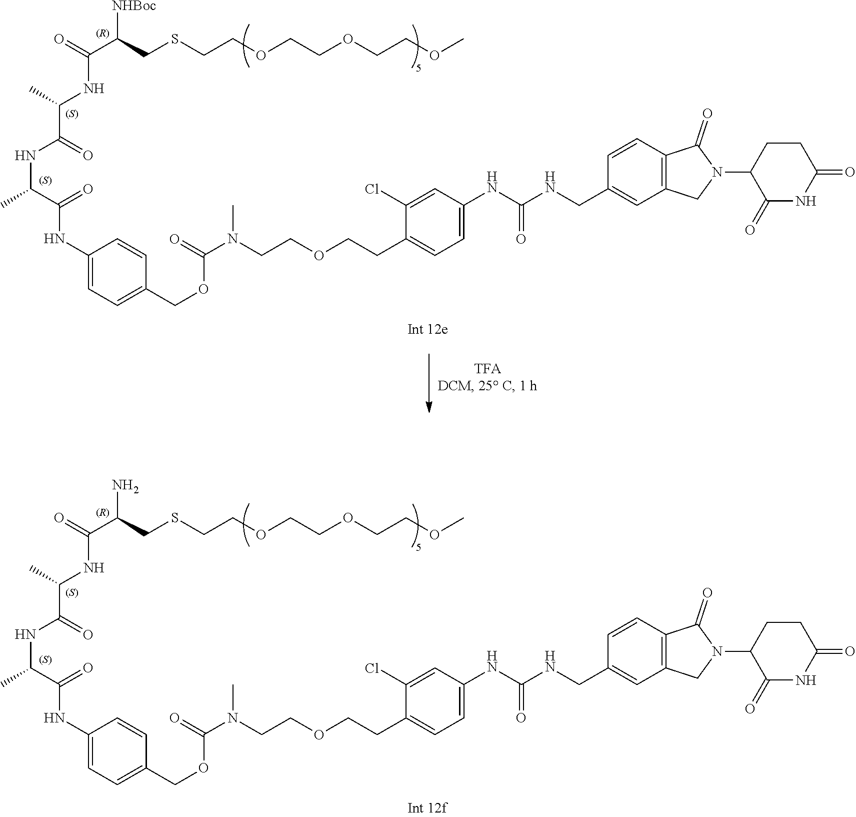

To a solution of 4-((37R,40S,43S)-37-((tert-butoxycarbonyl)amino)-40,43-dimethyl-38,41-dioxo-2,5,8,11,14,17,20,23,26,29,32-undecaoxa-35-thia-39,42-diazatetratetracontan-44-amido)benzyl (2-(2-chloro-4-(3-((2-(2,6-dioxopiperidin-3-yl)-1-oxoisoindolin-5-yl)methyl)ureido)phenethoxy)ethyl)(methyl)carbamate, Int 12e, (300 mg, 197 μmol, 1.00 eq) in dichloromethane (3.00 mL) was added trifluoroacetic acid (0.600 mL). The mixture was stirred at 25° C. for 1 h. The mixture was concentrated under reduced pressure to give a residue. The residue was purified by reversed-phase HPLC (C18, 40 g; condition: water/acetonitrile=1/0-0/1, 0.1% formic acid) and lyophilized to afford 4-((37R,40S,43S)-37-amino-40,43-dimethyl-38,41-dioxo-2,5,8,11,14,17,20,23,26,29,32-undecaoxa-35-thia-39,42-diazatetratetracontan-44-amido)benzyl (2-(2-chloro-4-(3-((E)-3-(1-(2,6-dioxopiperidin-3-yl)-4-methyl-5-oxo-2,5-dihydro-1H-pyrrol-3-yl) allyl)ureido) phenethoxy)ethyl)(methyl)carbamate, Int 12f, (220 mg, 155 μmol, 78% yield) as yellow oil. ¹H NMR (400 MHz, DMSO-d₆) δ=10.98 (br d, J=4.3 Hz, 1H), 9.94 (s, 1H), 8.78 (s, 1H), 833-822 (m, 1H), 8.17 (d, J=72 Hz, 1H), 7.71-7.65 (m, 2H), 7.59 (br d, J=8.0 Hz, 2H), 7.51 (s, 1H), 7.44 (d, J=7.6 Hz, 1H), 7.29 (br d, J=7.2 Hz, 2H), 7.21-7.11 (m, 2H), 6.81 (t, J=6.0 Hz, 1H), 5.10 (dd, J=5.2, 13.2 Hz, 1H), 4.98 (s, 2H), 4.47-4.28 (m, 7H), 3.50 (s, 48H), 3.43-3.42 (m, 2H), 3.37-3.34 (m, 2H), 3.23 (s, 3H), 2.96-2.87 (m, 1H), 2.83 (br d, J=8.4 Hz, 5H), 2.69-2.67 (m, 2H), 2.62-2.56 (m, 1H), 2.42-2.34 (m, 1H), 2.03-1.95 (m, 1H), 1.30 (d, J=7.2 Hz, 3H), 1.24 (d, J=7.0 Hz, 3H).

### Step G. Preparation of Compound IG-L-18

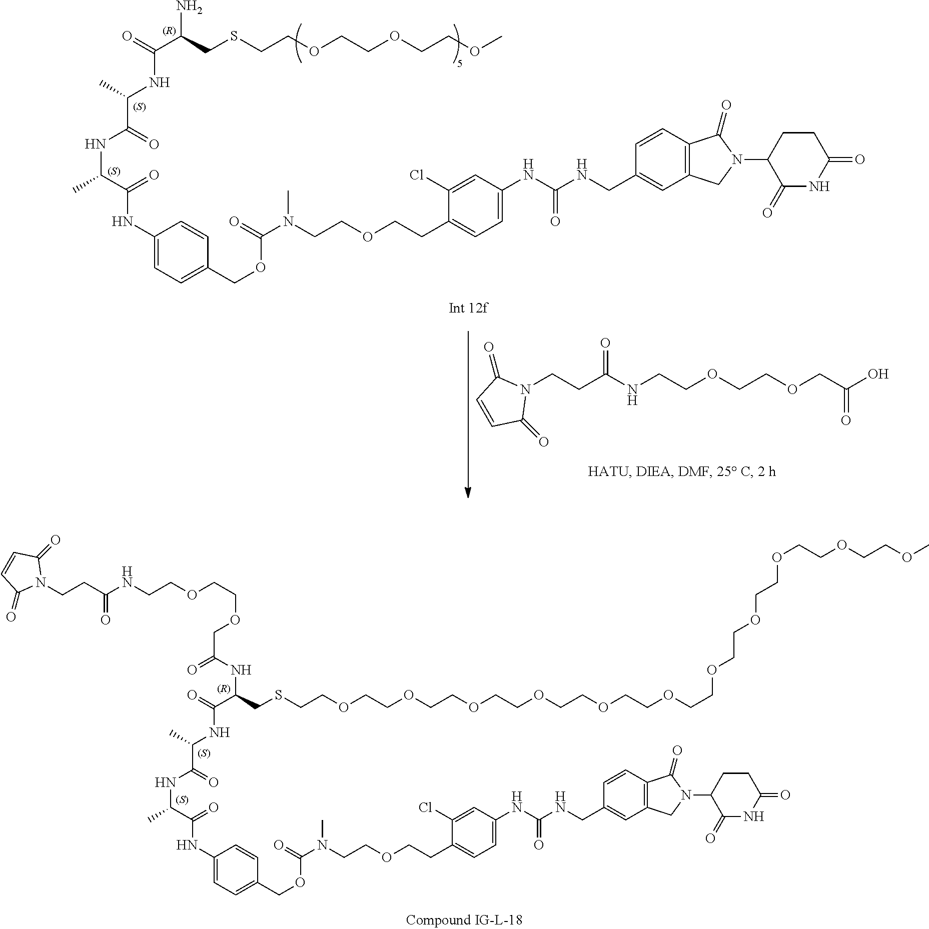

To a solution of 4-((37R,40S,43S)-37-amino-40,43-dimethyl-38,41-dioxo-2,5,8,11,14,17,20,23,26,29,32-undecaoxa-35-thia-39,42-diazatetratetracontan-44-amido)benzyl (2-(2-chloro-4-(3-((E)-3-(1-(2,6-dioxopiperidin-3-yl)-4-methyl-5-oxo-2,5-dihydro-1H-pyrrol-3-yl)allyl)ureido)phenethoxy)ethyl)(methyl) carbamate, Int 12f, (20.0 mg, 14.1 μmol, 1.00 eq) in dimethyl formamide (2.00 mL) was added O-(7-Azabenzotriazol-1-yl)-N,N,N′,N′-tetramethyluronium Hexafluorophosphate (5.89 mg, 15.5 μmol, 1.10 eq), 2-(2-(2-(3-(2,5-dioxo-2,5-dihydro-TH-pyrrol-1-yl)propanamido)ethoxy)ethoxy)acetic acid (5.31 mg, 16.9 μmol, 1.20 eq) and N,N-diisopropylethylamine (3.64 mg, 28.2 μmol, 4.90 μL, 2.00 eq). The mixture was stirred at 25° C. for 2 h. The mixture was concentrated under reduced pressure to give a residue. The residue was purified by prep-HPLC (column: Waters Xbridge 150*25 mm*5 um; mobile phase: [Water-ACN];gradient: 23%-53% B over 10 min). The desired fraction was collected and lyophilized to afford 4-((37R,40S,43S)-37-(2-(2-(2-(3-(2,5-dioxo-2,5-dihydro-1H-pyrrol-1-yl)propanamido)ethoxy) ethoxy)acetamido)-40,43-dimethyl-38,41-dioxo-2,5,8,11,14,17,20,23,26,29,32-undecaoxa-35-thia-39,42-diazatetratetracontan-44-amido)benzyl (2-(2-chloro-4-(3-((2-(2,6-dioxopiperidin-3-yl)-1-oxoisoindolin-5-yl) methyl)ureido)phenethoxy)ethyl)(methyl) carbamate, Compound IG-L-18, (5.30 mg, 2.96 mol, 21% yield, 96% purity) as yellow oil. ¹H NMR (400 MHz, DMSO-d₆) δ=10.98 (s, 1H), 9.95 (s, 1H), 8.78 (s, 1H), 8.42-8.32 (m, 1H), 8.08-7.98 (m, 2H), 7.77 (br d, J=8.8 Hz, 1H), 7.73-7.64 (m, 2H), 7.59 (br d, J=8.4 Hz, 2H), 7.52 (s, 1H), 7.44 (d, J=8.0 Hz, 1H), 7.29 (br d, J=7.6 Hz, 2H), 7.21-7.13 (m, 2H), 6.99 (s, 1H), 6.81 (t, J=6.0 Hz, 1H), 5.11 (dd, J=5.2, 13.3 Hz, 1H), 4.99 (s, 2H), 4.59-4.50 (m, 1H), 4.49-4.27 (m, 7H), 3.94 (d, J=1.4 Hz, 2H), 3.61-3.50 (m, 48H), 3.43 (dd, J=3.2, 5.8 Hz, 4H), 3.24 (s, 3H), 3.18 (br dd, J=5.2, 9.8 Hz, 2H), 2.94-2.79 (m, 8H), 2.77-2.59 (m, 7H), 2.40-2.31 (m, 3H), 2.03-1.97 (m, 1H), 1.30 (d, J=7.2 Hz, 3H), 1.24 (br d, J=7.2 Hz, 3H). MS (ESI) m/z 1718.1 [M+H]⁺

### Example L-19: Synthesis of 4-((37S,40S,43S)-37-(2-(2-(2-(3-(2,5-dioxo-2,5-dihydro-1H-pyrrol-1-yl)propanamido)ethoxy)ethoxy)acetamido)-40,43-dimethyl-31,38,41-trioxo-2,5,8,11,14,17,20,23,26,29-decaoxa-32,39,42-triazatetratetracontan-44-amido)benzyl (2-(2-chloro-4-(3-((2-(2,6-dioxopiperidin-3-yl)-1-oxoisoindolin-5-yl)methyl)ureido)phenethoxy)ethyl)(methyl)carbamate (IG-L-19)

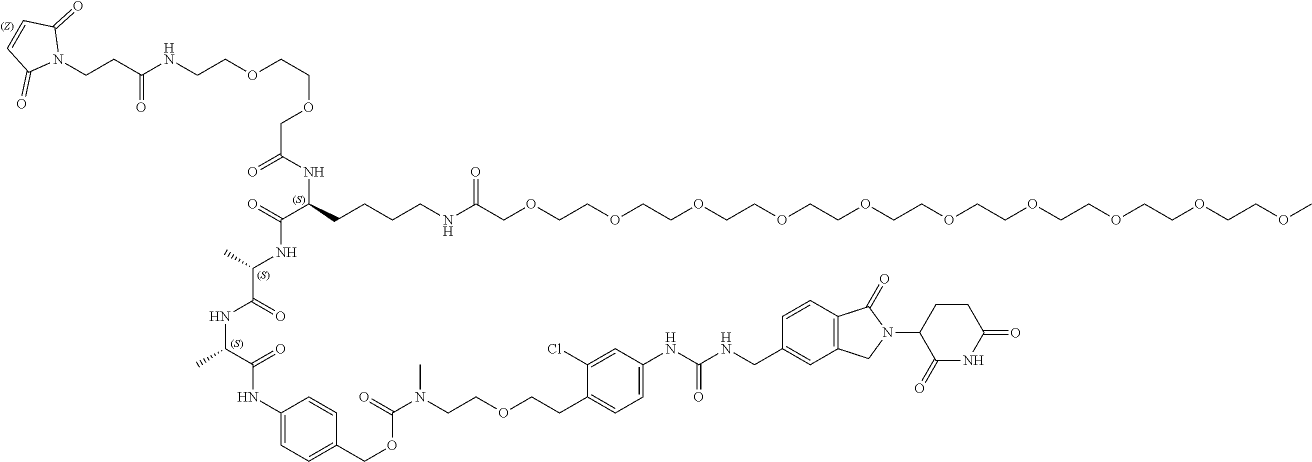

### Step A. Preparation of Int 13a

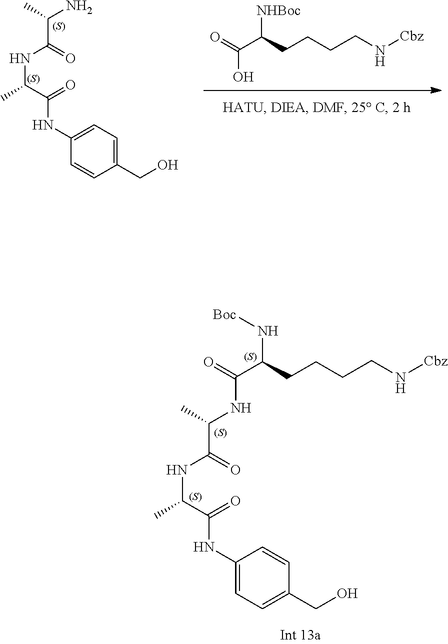

To a solution of (S)-2-amino-N—((S)-1-((4-(hydroxymethyl)phenyl)amino)-1-oxopropan-2-yl)propanamide (1.27 g, 4.79 mmol, 1.00 eq) in dimethyl formamide (8.00 mL) was added N⁶-((benzyloxy)carbonyl)-N²-(tert-butoxycarbonyl)-L-lysine (2.00 g, 5.27 mmol, 1.10 eq), 0-(7-Azabenzotriazol-1-yl)-N,N,N′,N′-tetramethyluronium Hexafluorophosphate (2.37 g, 6.22 mmol, 1.30 eq) and NN-diisopropylethylamine (1.24 g, 9.57 mmol, 1.67 mL, 2.00 eq). The mixture was stirred at 25° C. for 2 h. The reaction mixture was filtered to give the filtrate. The filtrate was purified by reversed-phase HPLC (0.1% FA) to give benzyl tert-butyl ((S)-6-(((S)-1-(((S)-1-((4-(hydroxymethyl)phenyl)amino)-1-oxopropan-2-yl)amino)-1-oxopropan-2-yl)amino)-6-oxohexane-1,5-diyl)dicarbamate, Int 13a, (1.90 g, 3.03 mmol, 63% yield) as a white solid. ¹H NMR (400 MHz, DMSO-d₆) δ=9.85 (s, 1H), 8.08 (br d, J=7.2 Hz, 1H), 7.89 (br d, J=7.2 Hz, 1H), 7.54 (br d, J=8.4 Hz, 2H), 7.38-7.30 (m, 5H), 7.23 (br d, J=8.4 Hz, 3H), 6.91 (br d, J=7.6 Hz, 1H), 5.75 (s, 2H), 5.09 (t, J=5.6 Hz, 1H), 4.99 (s, 2H), 4.42 (d, J=5.6 Hz, 2H), 4.37 (br t, J=7.2 Hz, 1H), 4.28 (br t, J=7.2 Hz, 1H), 3.91-3.79 (m, 1H), 2.96 (br d, J=5.6 Hz, 2H), 1.67-1.54 (m, 1H), 1.49-1.43 (m, 1H), 1.37 (s, 1H), 1.29 (br d, J=7.2 Hz, 3H), 1.21 (d, J=7.2 Hz, 3H). MS (ESI) m/z 510.3 [M+H-100-18]⁺

### Step B. Preparation of Int 13b

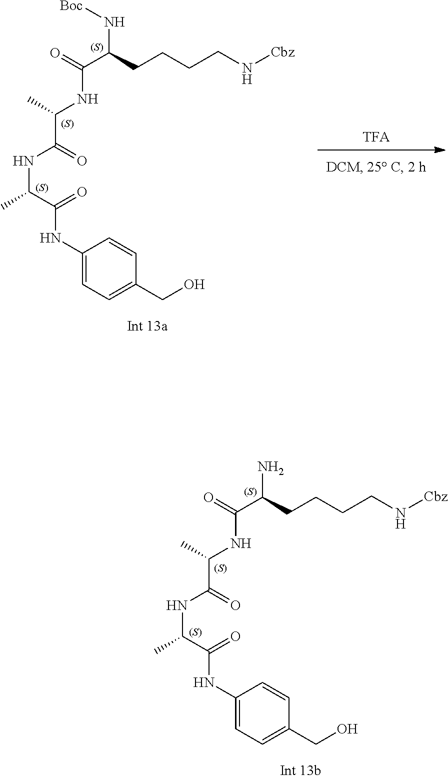

To a solution of benzyl tert-butyl ((S)-6-(((S)-1-(((S)-1-((4-(hydroxymethyl)phenyl)amino)-1-oxopropan-2-yl)amino)-1-oxopropan-2-yl)amino)-6-oxohexane-1,5-diyl)dicarbamate, Int 13a, (1.80 g, 2.87 mmol, 1.00 eq) in dichloromethane (20.0 mL) was added trifluoroacetic acid (2.00 mL). The mixture was stirred at 25° C. for 2 h. The reaction mixture was concentrated under reduced pressure to give a residue. The residue was purified by reversed-phase HPLC (0.1% FA) to give benzyl ((S)-5-amino-6-(((S)-1-(((S)-1-((4-(hydroxymethyl)phenyl)amino)-1-oxopropan-2-yl)amino)-1-oxopropan-2-yl)amino)-6-oxohexyl) carbamate, Int 13b, (1.10 g, 682 μmol, 66% yield, 90% purity) as a yellow solid. ¹H NMR (400 MHz, DMSO-d₆) δ=9.96 (s, 1H), 8.46 (br d, J=7.2 Hz, 1H), 8.22 (d, J=7.2 Hz, 1H), 7.54 (d, J=8.4 Hz, 2H), 7.39-7.29 (m, 5H), 7.27-7.19 (m, 3H), 5.00 (s, 3H), 4.47-4.35 (m, 4H), 3.58 (br t, J=6.4 Hz, 2H), 2.97 (q, J=6.4 Hz, 2H), 1.71-1.52 (m, 2H), 1.45-1.19 (m, 10H). MS (ESI) m/z 528.3 [M+H]⁺

### Step C. Preparation of Int 13c

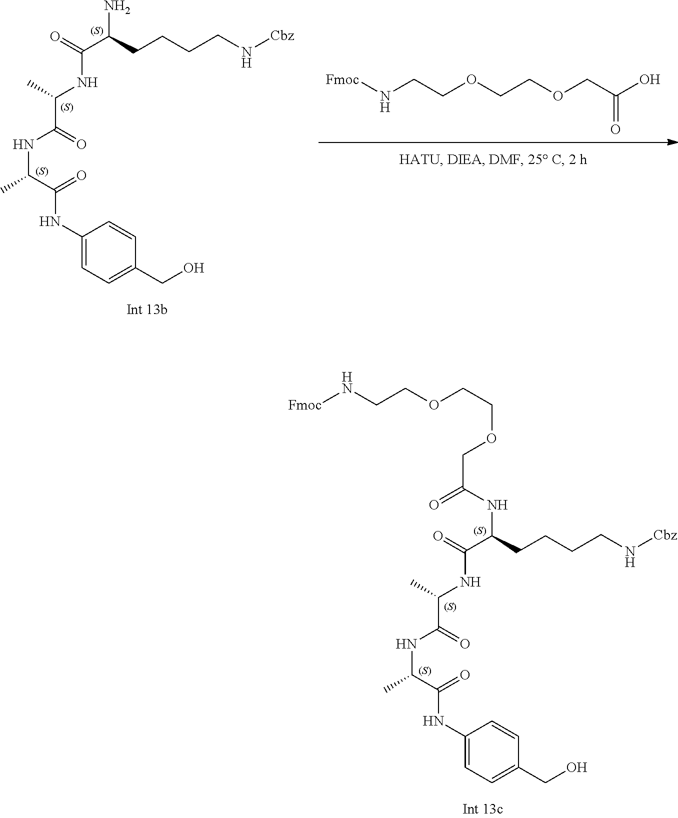

To a solution of benzyl ((S)-5-amino-6-(((S)-1-(((S)-1-((4-(hydroxymethyl)phenyl)amino)-1-oxopropan-2-yl)amino)-1-oxopropan-2-yl)amino)-6-oxohexyl)carbamate, Int 13b, (1.10 g, 2.08 mmol, 1.00 eq) in dimethyl formamide (3.00 mL) was added 1-(9H-fluoren-9-yl)-3-oxo-2,7,10-trioxa-4-azadodecan-12-oic acid (884 mg, 2.29 mmol, 1.10 eq), 0-(7-Azabenzotriazol-1-yl)-N,N,N′,N′-tetramethyluronium Hexafluorophosphate (1.19 g, 3.13 mmol, 1.50 eq) and N,N-diisopropylethylamine (808 mg, 6.25 mmol, 1.09 mL, 3.00 eq). The mixture was stirred at 25° C. for 2 h. The reaction mixture was filtered and the filtrate was purified by reversed-phase HPLC (0.1% FA) to give (9H-fluoren-9-yl)methyl ((S)-9-(((S)-1-(((S)-1-((4-(hydroxymethyl)phenyl)amino)-1-oxopropan-2-yl)amino)-1-oxopropan-2-yl) carbamoyl)-3,11-dioxo-1-phenyl-2,13,16-trioxa-4,10-diazaoctadecan-18-yl)carbamate, Int 13c, (1.05 g, 1.06 mmol, 51% yield, 90% purity) as a white solid. ¹H NMR (400 MHz, DMSO-d₆) δ=9.88 (s, 1H), 8.21 (br d, J=7.2 Hz, 1H), 8.04 (d, J=7.2 Hz, 1H), 7.88 (d, J=7.6 Hz, 2H), 7.68 (d, J=7.6 Hz, 2H), 7.62 (br d, J=7.6 Hz, 1H), 7.54 (d, J=8.4 Hz, 2H), 7.43-7.38 (m, 2H), 7.36-7.29 (m, 8H), 7.25-7.19 (m, 3H), 5.10 (t, J=5.6 Hz, 1H), 4.98 (s, 2H), 4.42 (d, J=5.6 Hz, 2H), 4.38-4.26 (m, 5H), 4.23-4.18 (m, 1H), 3.91 (s, 2H), 3.60-3.52 (m, 4H), 3.44-3.40 (m, 2H), 3.17-3.12 (m, 2H), 2.95 (br d, J=6.0 Hz, 2H), 1.70-1.62 (m, 1H), 1.58-1.50 (m, 1H), 1.37 (br d, J=7.2 Hz, 2H), 1.30-1.21 (m, 8H). MS (ESI) m/z 877.3 [M+H-18]⁺

### Step D. Preparation of Int 13d

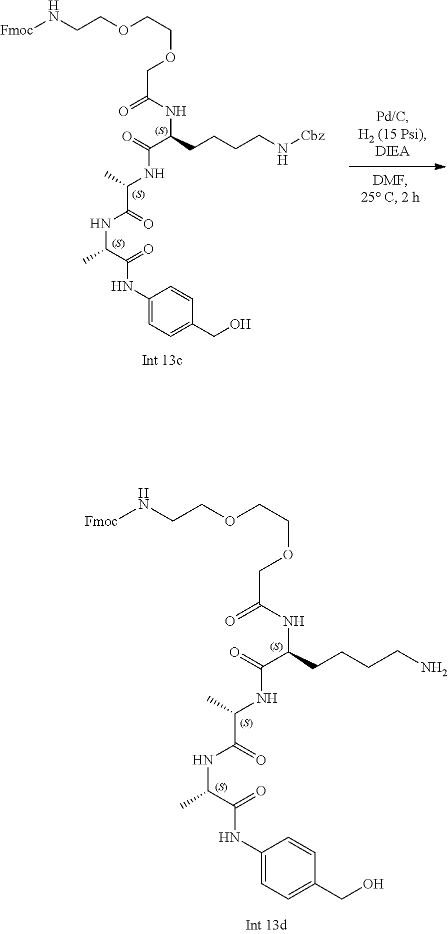

To a solution of (9H-fluoren-9-yl)methyl ((S)-9-(((S)-1-(((S)-1-((4-(hydroxymethyl)phenyl)amino)-1-oxopropan-2-yl)amino)-1-oxopropan-2-yl)carbamoyl)-3,11-dioxo-1-phenyl-2,13,16-trioxa-4,10-diazaoctadecan-18-yl)carbamate, Int 13c, (300 mg, 335 umol, 1.00 eq) in dimethyl formamide (5.00 mL) was added N,N-diisopropylethylamine (86.6 mg, 670 μmol, 117 μL, 2.00 eq) and palladium on carbon (100 mg, 10% purity) under nitrogen. The suspension was degassed under vacuum and purged with hydrogen several times. The mixture was stirred under hydrogen (15 psi) at 25° C. for 2 h. The reaction mixture was filtered and the filtrate was concentrated under vacuum to give a residue. The residue was purified by reversed-phase HPLC(H₂O/ACN) to give (9H-fluoren-9-yl)methyl((2S,5S,8S)-8-(4-aminobutyl)-1-((4-(hydroxymethyl)phenyl)amino)-2,5-dimethyl-1,4,7,10-tetraoxo-12,15-dioxa-3,6,9-triazaheptadecan-17-yl)carbamate, Int 13d, (250 mg, 328 μmol, 98% yield) as a yellow solid. ¹H NMR (400 MHz, DMSO-d₆) δ=9.90 (s, 1H), 8.22 (br d, J=7.2 Hz, 1H), 8.09 (br d, J=6.8 Hz, 1H), 7.89 (d, J=7.4 Hz, 2H), 7.70-7.64 (m, 3H), 7.53 (br d, J=8.4 Hz, 2H), 7.40 (br d, J=7.2 Hz, 2H), 7.36-7.30 (m, 3H), 7.24 (br d, J=8.4 Hz, 2H), 5.28-4.96 (m, 1H), 4.43 (s, 2H), 4.38-4.26 (m, 5H), 4.24-4.17 (m, 1H), 3.94-3.90 (m, 2H), 3.61-3.53 (m, 4H), 3.42 (br d, J=6.0 Hz, 2H), 3.18-3.12 (m, 2H), 2.73 (br t, J=7.2 Hz, 2H), 1.73-1.64 (m, 1H), 1.57-1.44 (m, 3H), 1.34-1.19 (m, 9H), 1.05 (br d, J=3.6 Hz, 1H). MS (ESI) m/z 761.3 [M+H]⁺

### Step E. Preparation of Int 13e

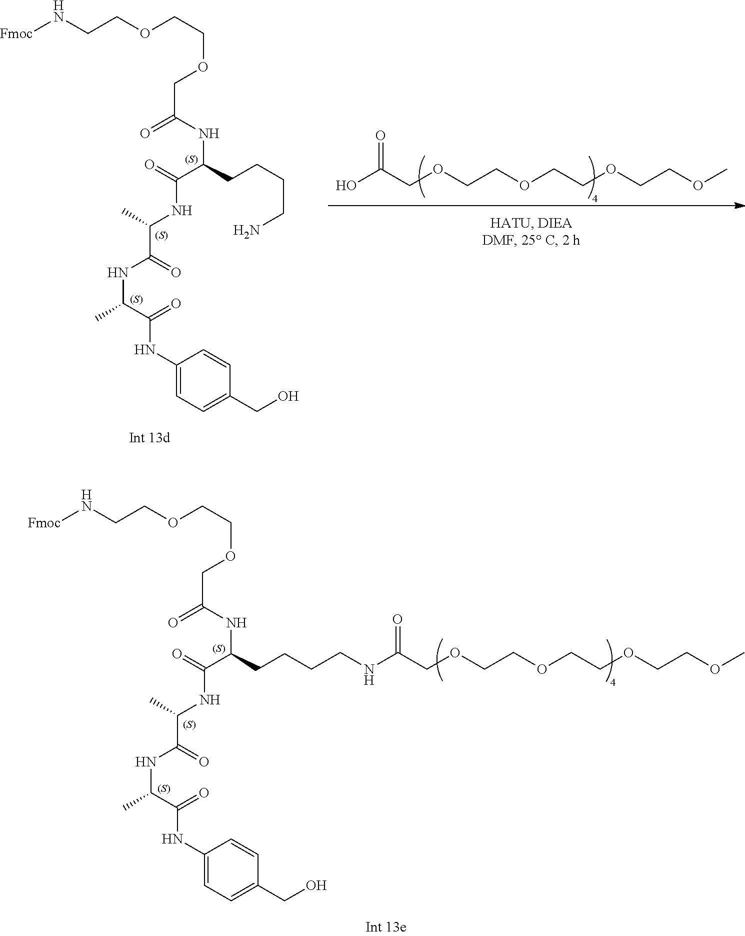

To a solution of2,5,8,11,14,17,20,23,26,29-decaoxahentriacontan-31-oic acid (160 mg, 329 μmol, 1.00 eq) in dimethyl formamide (3 mL) was added O-(7-Azabenzotriazol-1-yl)-N,N,N′,N′-tetramethyluronium Hexafluorophosphate (187 mg, 493 μmol, 1.50 eq), N,N-diisopropylethylamine (127 mg, 986 μmol, 172 μL, 3.00 eq) and (9H-fluoren-9-yl)methyl ((2S,5S,8S)-8-(4-aminobutyl)-1-((4-(hydroxymethyl)phenyl)amino)-2,5-dimethyl-1,4,7,10-tetraoxo-12,15-dioxa-3,6,9-triazaheptadecan-17-yl)carbamate, Int 13d, (250 mg, 328 μmol, 1.00 eq). The mixture was stirred at 25° C. for 2 h. The reaction mixture was filtered to give filtrate. The filtrate was purified by reversed-phase HPLC (0.1% FA) to give (9H-fluoren-9-yl)methyl ((S)-37-(((S)-1-(((S)-1-((4-(hydroxymethyl)phenyl)amino)-1-oxopropan-2-yl)amino)-1-oxopropan-2-yl)carbamoyl)-31,39-dioxo-2,5,8,11,14,17,20,23,26,29,41,44-dodecaoxa-32,38-diazahexatetracontan-46-yl)carbamate, Int 13e, (280 mg, 228 μmol, 69% yield) as yellow oil. ¹H NMR (400 MHz, DMSO-d₆) δ=9.88 (s, 1H), 8.20 (br d, J=7.2 Hz, 1H), 8.03 (d, J=7.2 Hz, 1H), 7.88 (d, J=7.6 Hz, 2H), 7.68 (br d, J=7.6 Hz, 2H), 7.65-7.58 (m, 2H), 7.54 (d, J=8.4 Hz, 2H), 7.44-7.38 (m, 2H), 7.37-7.28 (m, 3H), 7.23 (d, J=8.4 Hz, 2H), 5.09 (t, J=5.6 Hz, 1H), 4.44-4.26 (m, 7H), 4.24-4.16 (m, 1H), 3.91 (s, 2H), 3.87-3.77 (m, 2H), 3.58 (br d, J=4.0 Hz, 2H), 3.57-3.52 (m, 6H), 3.52-3.46 (m, 27H), 3.45-3.39 (m, 4H), 3.23 (s, 3H), 3.15 (q, J=6.0 Hz, 2H), 3.09-3.02 (m, 2H), 1.71-1.61 (m, 1H), 1.59-1.50 (m, 1H), 1.43-1.35 (m, 2H), 1.32-1.19 (m, 8H). MS (ESI) m/z 1211.5 [M+H-18]⁺

### Step F. Preparation of Int 13f

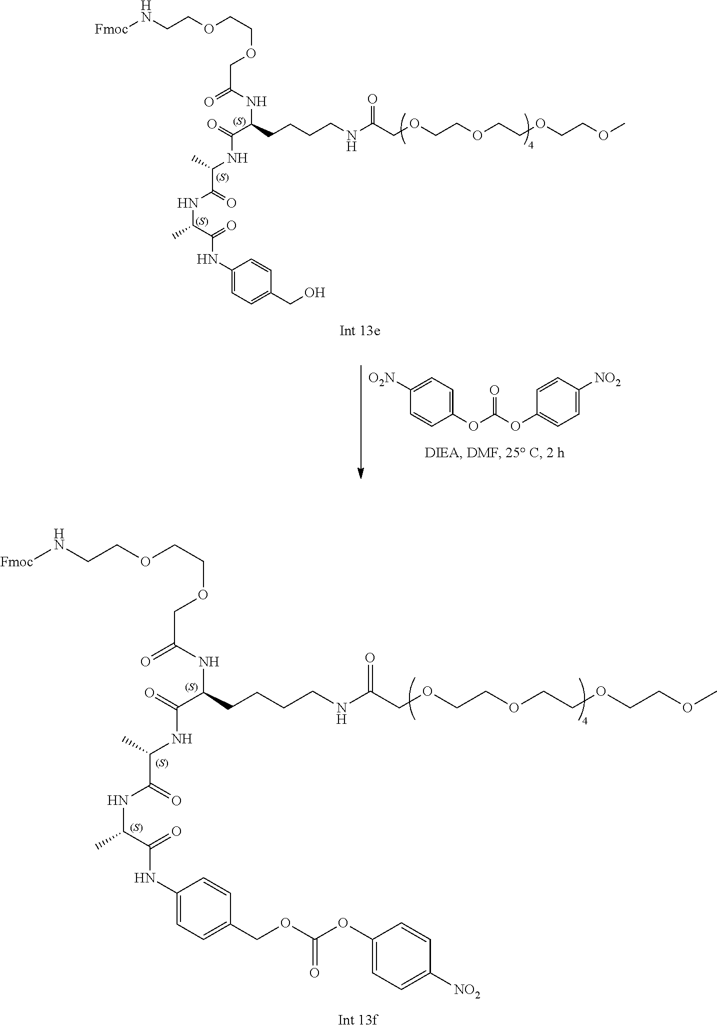

To a solution of (9H-fluoren-9-yl)methyl ((S)-37-(((S)-1-(((S)-1-((4-(hydroxymethyl)phenyl)amino)-1-oxopropan-2-yl)amino)-1-oxopropan-2-yl)carbamoyl)-31,39-dioxo-2,5,8,11,14,17,20,23,26,29,41,44-dodecaoxa-32,38-diazahexatetracontan-46-yl)carbamate, Int 13e, (180 mg, 146 μmol, 1.00 eq) in dimethyl formamide (2.00 mL) was added bis(4-nitrophenyl) carbonate (53.5 mg, 176 μmol, 1.20 eq) and N,N-diisopropylethylamine (28.4 mg, 220 μmol, 38.3 μL, 1.5 eq). The mixture was stirred at 25° C. for 2 h. The reaction mixture was filtered to give filtrate. The filtrate was purified by reversed-phase HPLC (0.1% FA) to (9H-fluoren-9-yl)methyl ((S)-37-(((S)-1-(((S)-1-((4-((((4-nitrophenoxy)carbonyl)oxy)methyl)phenyl) amino)-1-oxopropan-2-yl)amino)-1-oxopropan-2-yl)carbamoyl)-31,39-dioxo-2,5,8,11,14,17,20,23,26,29,41,44-dodecaoxa-32,38-diazahexatetracontan-46-yl)carbamate, Int 13f, (100 mg, 68.1 μmol, 47% yield, 95% purity) as colorless oil. MS (ESI) m/z 1394.6 [M+H]⁺

### Step G. Preparation of Int 13g

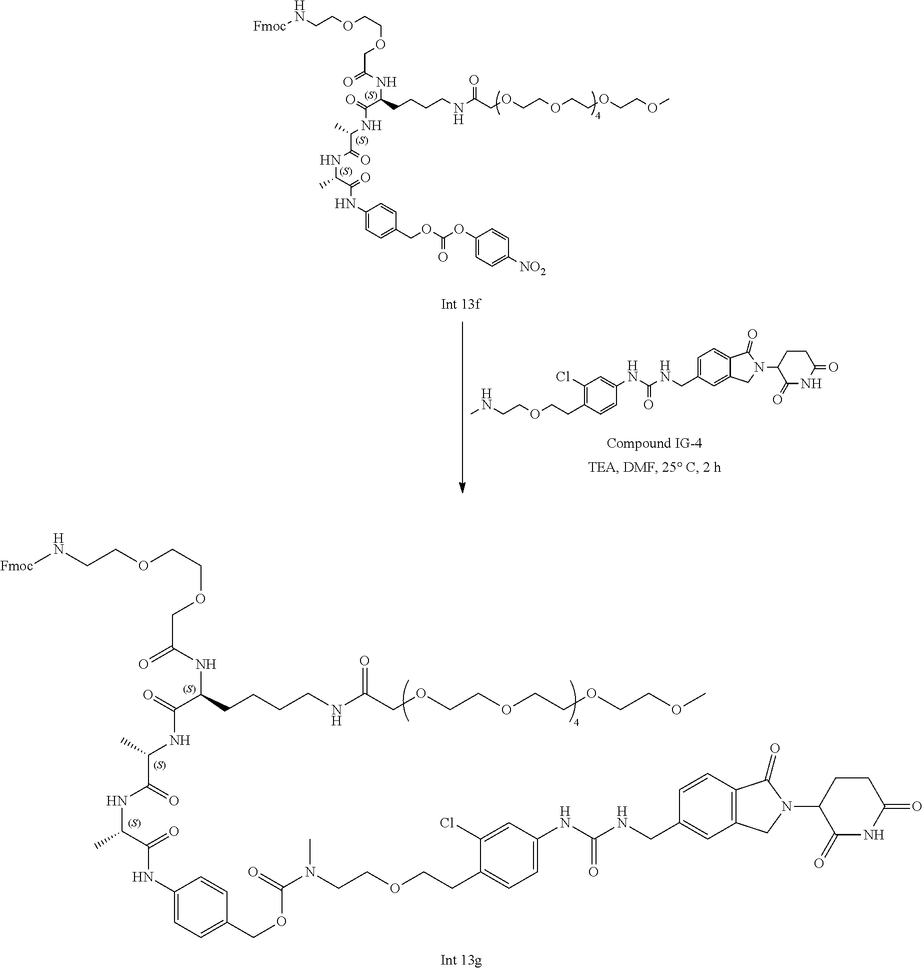

To a solution of (9H-fluoren-9-yl)methyl ((S)-37-(((S)-1-(((S)-1-((4-((((4-nitrophenoxy)carbonyl)oxy)methyl) phenyl)amino)-1-oxopropan-2-yl)amino)-1-oxopropan-2-yl)carbamoyl)-31,39-dioxo-2,5,8,11,14,17,20,23,26,29,41,44-dodecaoxa-32,38-diazahexatetracontan-46-yl)carbamate, Int 13f, (80.0 mg, 57.4 μmol, 1.00 eq) in dimethyl formamide (1.00 mL) was added 1-(3-chloro-4-(2-(2-(methylamino)ethoxy)ethyl)phenyl)-3-((2-(2,6-dioxopiperidin-3-yl)-1-oxoisoindolin-5-yl)methyl)urea, Compound IG-4, (30.3 mg, 57.4 μmol, 1.00 eq) and triethylamine (17.4 mg, 172 gmol, 24.0 μL, 3.00 eq). The mixture was stirred at 25° C. for 2 h. The reaction mixture was filtered to give filtrate. The crude product was purified by reversed-phase HPLC (0.1% FA) to give 4-((37S,40S,43S)-37-(1-(9H-fluoren-9-yl)-3-oxo-2,7,10-trioxa-4-azadodecan-12-amido)-40,43-dimethyl-31,38,41-trioxo-2,5,8,11,14,17,20,23,26,29-decaoxa-32,39,42-triazatetratetracontan-44-amido)benzyl (2-(2-chloro-4-(3-((2-(2,6-dioxopiperidin-3-yl)-1-oxoisoindolin-5-yl)methyl)ureido)phenethoxy)ethyl)(methyl)carbamate, Int 13g, (40.0 mg, 19.3 μmol, 34% yield, 86% purity) as a white solid. MS (ESI) m/z 1783.8 [M+H]⁺

### Step H. Preparation of Int 13h

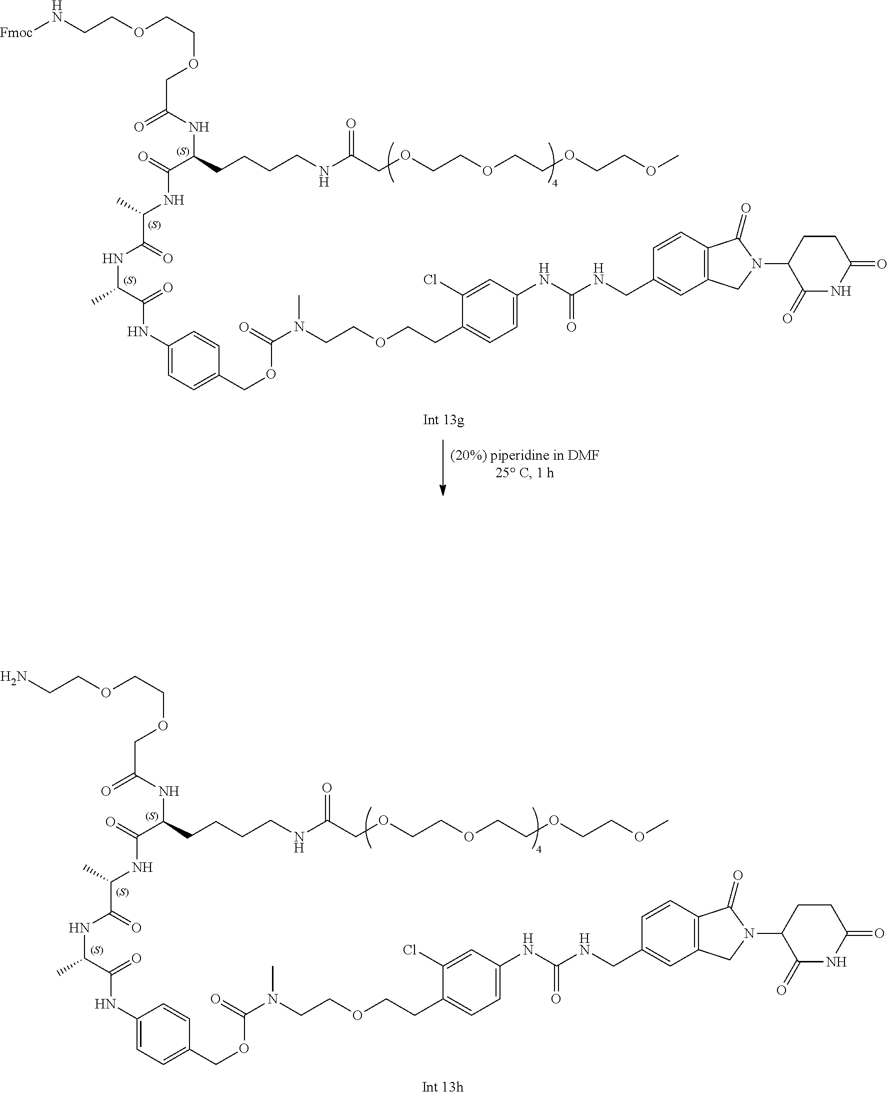

To a solution of4-((37S,40S,43S)-37-(1-(9H-fluoren-9-yl)-3-oxo-2,7,10-trioxa-4-azadodecan-12-amido)-40,43-dimethyl-31,38,41-trioxo-2,5,8,11,14,17,20,23,26,29-decaoxa-32,39,42-triazatetratetracontan-44-amido)benzyl(2-(2-chloro-4-(3-((2-(2,6-dioxopiperidin-3-yl)-1-oxoisoindolin-5-yl) methyl)ureido) phenethoxy)ethyl)(methyl)carbamate, Int 13g, (40.0 mg, 22.4 μmol, 1.00 eq) in dimethyl formamide (2.00 mL) was added piperidine (0.4 mL). The mixture was stirred at 25° C. for 1 h. The reaction mixture was filtered and the filtrate was purified by reversed-phase HPLC (H₂O/ACN) to give 4-((37S,40S,43S)-37-(2-(2-(2-aminoethoxy)ethoxy)acetamido)-40,43-dimethyl-31,38,41-trioxo-2,5,8,11,14,17,20,23,26,29-decaoxa-32,39,42-triazatetratetracontan-44-amido)benzyl (2-(2-chloro-4-(3-((2-(2,6-dioxopiperidin-3-yl)-1-oxoisoindolin-5-yl)methyl)ureido)phenethoxy)ethyl)(methyl)carbamate, Int 13h, (30.0 mg, 12.1 μmol, 54% yield, 63% purity) as a yellow solid. MS (ESI) m/z 1560.8 [M+H]⁺

### Step I. Preparation of Compound IG-L-19

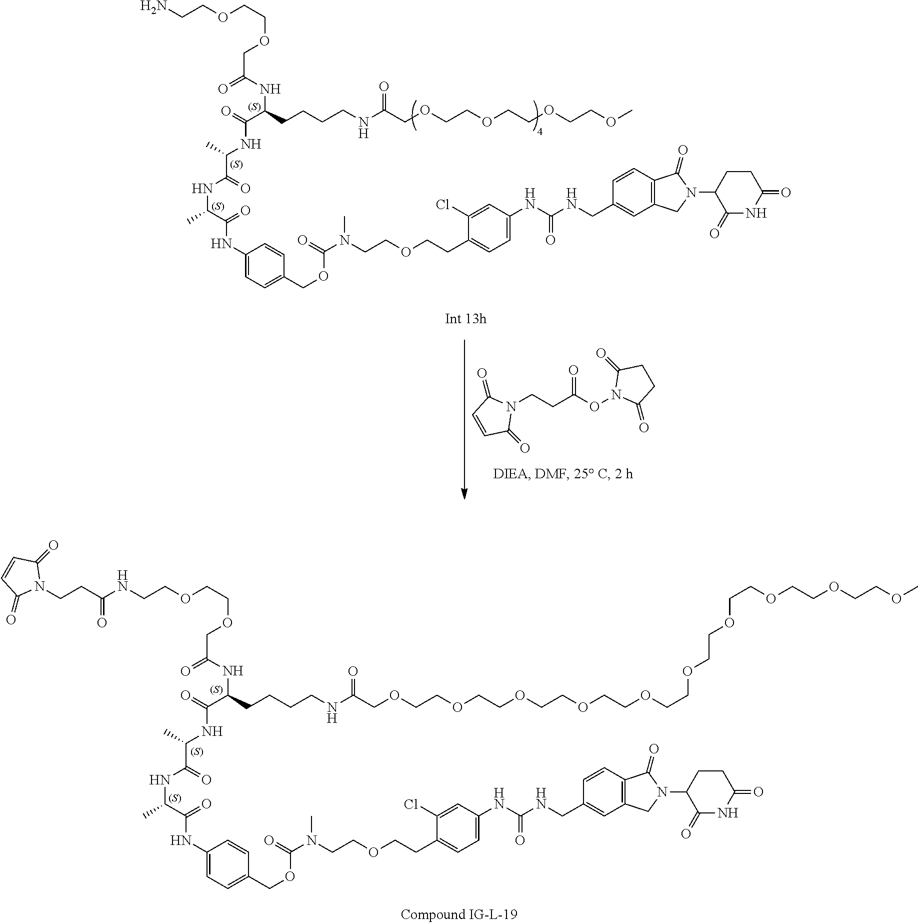

To a solution of 4-((37S,40S,43S)-37-(2-(2-(2-aminoethoxy)ethoxy)acetamido)-40,43-dimethyl-31,38,41-trioxo-2,5,8,11,14,17,20,23,26,29-decaoxa-32,39,42-triazatetratetracontan-44-amido)benzyl (2-(2-chloro-4-(3-((2-(2,6-dioxopiperidin-3-yl)-1-oxoisoindolin-5-yl) methyl)ureido)phenethoxy)ethyl)(methyl)carbamate, Int 13h, (30.0 mg, 19.2 μmol, 1.00 eq) in dimethyl formamide (0.50 mL) was added (2,5-dioxopyrrolidin-1-yl) 3-(2,5-dioxopyrrol-1-yl)propanoate (5.12 mg, 19.2 μmol, 1.00 eq) and N,N-diisopropylethylamine (4.97 mg, 38.4 mol, 6.69 μL, 2.00 eq). The mixture was stirred at 25° C. for 2 h. The reaction mixture was filtered to give filtrate. The residue was purified by prep-HPLC (column: Waters Xbridge 150*25 mm*5 um; mobile phase: [Water-ACN]; gradient: 25%-55% B over 9 min) to give 4-((37S,40S,43 S)-37-(2-(2-(2-(3-(2,5-dioxo-2,5-dihydro-1H-pyrrol-1-yl)propanamido)ethoxy)ethoxy)acetamido)-40,43-dimethyl-31,38,41-trioxo-2,5,8,11,14, 17,20,23,26,29-decaoxa-32,39,42-triazatetratetracontan-44-amido)benzyl (2-(2-chloro-4-(3-((2-(2,6-dioxopiperidin-3-yl)-1-oxoisoindolin-5-yl)methyl)ureido)phenethoxy)ethyl)(methyl)carbamate, Compound IG-L-19, (19.2 mg, 11.0 mol, 57% yield, 97% purity) as a yellow solid. ¹H NMR (400 MHz, DMSO-d₆) δ=10.96 (br s, 1H), 9.96 (s, 1H), 8.77 (s, 1H), 8.20 (br d, J=7.2 Hz, 1H), 8.07-7.97 (m, 2H), 7.72-7.65 (m, 2H), 7.64-7.55 (m, 4H), 7.51 (s, 1H), 7.44 (d, J=8.0 Hz, 1H), 7.28 (br d, J=7.6 Hz, 2H), 7.19-7.11 (m, 2H), 6.99 (s, 2H), 6.80 (br t, J=5.6 Hz, 1H), 5.10 (dd, J=5.2, 13.2 Hz, 1H), 4.98 (s, 2H), 4.47-4.39 (m, 3H), 4.38-4.25 (m, 4H), 3.91 (s, 2H), 3.84 (s, 2H), 3.59 (br d, J=6.8 Hz, 4H), 3.55-3.47 (m, 41H), 3.44-3.35 (m, 6H), 3.23 (s, 3H), 3.19-3.13 (m, 2H), 3.06 (q, J=6.8 Hz, 2H), 2.96-2.80 (m, 6H), 2.63-2.56 (m, 1H), 241-231 (m, 3H), 2.02-1.96 (m, 1H), 1.71-1.62 (m, 1H), 1.58-1.50 (m, 1H), 1.40 (br dd, J=4.4, 6.8 Hz, 2H), 1.29 (br d, J=7.2 Hz, 3H), 1.22 (br d, J=7.2 Hz, 5H). MS (ESI) m/z 1711.8 [M+H]⁺

### Example L-14: Synthesis of (32S)-32-(2-(4-(2-(4-((S)-2-((S)-2-acetamidopropanamido)propanamido)phenyl)-2-(((2-(2-chloro-4-(3-((2-(2,6-dioxopiperidin-3-yl)-1-oxoisoindolin-5-yl)methyl)ureido)phenethoxy)ethyl)(methyl)carbamoyl)oxy)ethyl)-1H-1,2,3-triazol-1-yl)ethyl)-45-(2,5-dioxo-2,5-dihydro-1H-pyrrol-1-yl)-6,12,18,24,30-pentamethyl-7,13,19,25,31,34,43-heptaoxo-3,9,15,21,27,36,39-heptaoxa-6,12,18,24,30,33,42-heptaazapentatetracontanoic acid (IG-L-14)

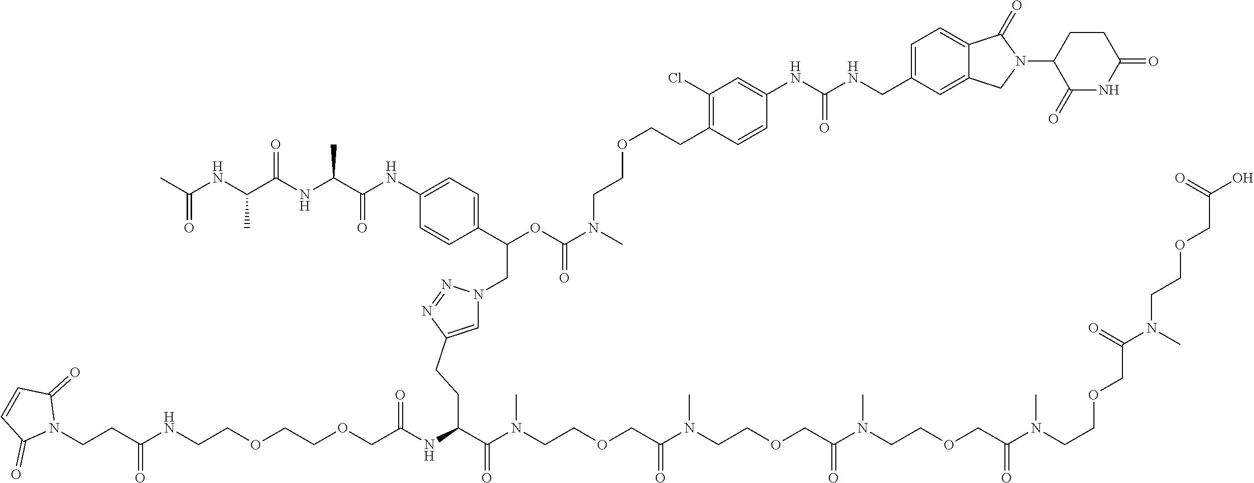 [SMILES: CC(=O)N[C@@H](C)C(=O)N[C@@H](C)C(=O)Nc1ccc(C(Cn2cc(CC[C@H](NC(=O)COCCOCCNC(=O)CCN3C(=O)C=CC3=O)C(=O)N(C)CCOCC(=O)N(C)CCOCC(=O)N(C)CCOCC(=O)N(C)CCOCC(=O)N(C)CCOCC(=O)O)nn2)OC(=O)N(C)CCOCCc2ccc(NC(=O)NCc3ccc4c(c3)CN(C3CCC(=O)NC3=O)C4=O)cc2Cl)cc1]

### Step A. Preparation of Int 14a

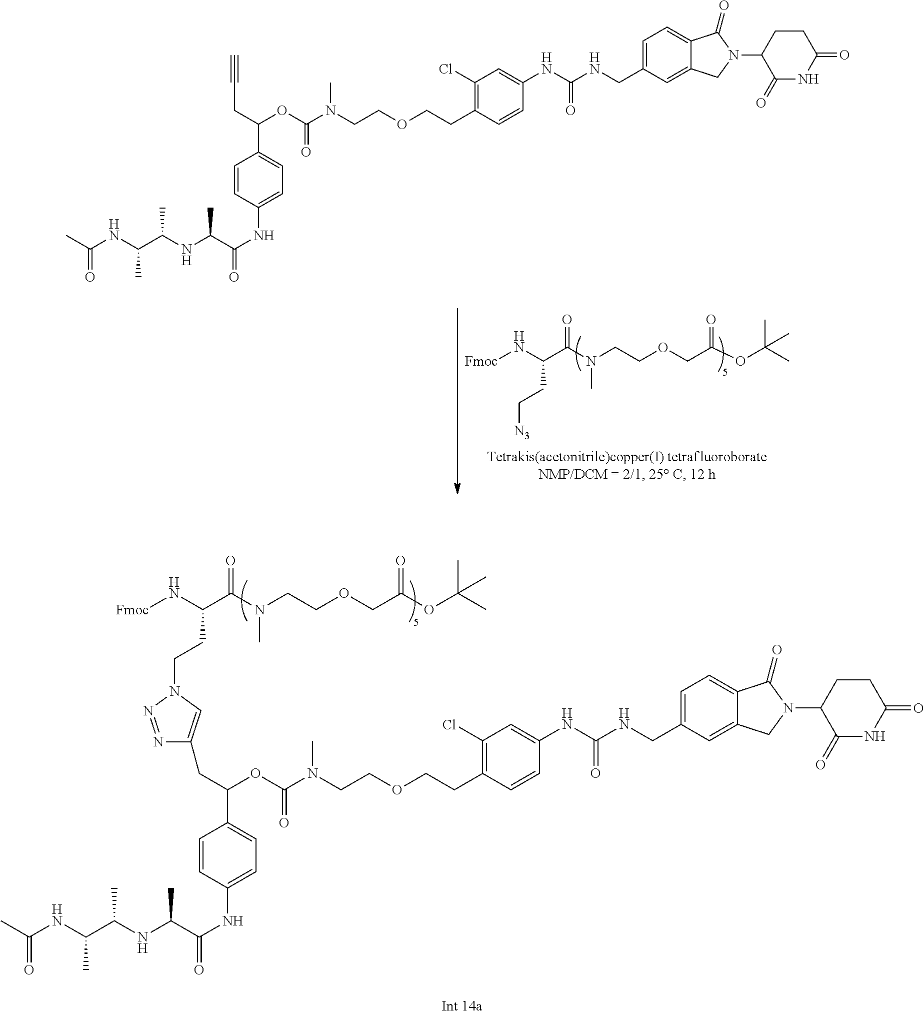

To a solution of 1-1-(4-((S)-2-((S)-2-acetamidopropanamido)propanamido)phenyl)but-3-yn-1-yl (2-(2-chloro-4-(3-((2-(2,6-dioxopiperidin-3-yl)-1-oxoisoindolin-5-yl)methyl)ureido)phenethoxy)ethyl)(methyl)carbamate (100 mg, 111 umol, 1.00 eq) in N-methyl pyrrolidone (1.50 mL) and dichloromethane (1.50 mL) was added tert-butyl (S)-5-(2-azidoethyl)-1-(9H-fluoren-9-yl)-7,13,19,25,31-pentamethyl-3,6,12,18,24,30-hexaoxo-2,10,16,22,28,34-hexaoxa-4,7,13,19,25,31-hexaazahexatriacontan-36-oate (122 mg, 122 umol, 1.10 eq) and cuprous;acetonitrile;tetrafluoroborate (105 mg, 334 umol, 3.00 eq). The mixture was stirred at 25° C. for 12 h. The reaction mixture was filtered and concentrated to remove the dichloromethane to afford tert-butyl (5S)-5-(2-(4-(2-(4-((S)-2-((S)-2-acetamidopropanamido)propanamido)phenyl)-2-(((2-(2-chloro-4-(3-((2-(2,6-dioxopiperidin-3-yl)-1-oxoisoindolin-5-yl)methyl)ureido)phenethoxy)ethyl) (methyl)carbamoyl)oxy)ethyl)-1H-1,2,3-triazol-1-yl)ethyl)-1-(9H-fluoren-9-yl)-7,13,19,25,31-pentamethyl-3,6,12,18,24,30-hexaoxo-2,10,16,22,28,34-hexaoxa-4,7,13,19,25,31-hexaazahexatriacontan-36-oate, Int 14a, (200 mg, crude) as yellow oil. MS (ESI) m/z 949.6 [1/2 M+H]⁺

### Step B. Preparation of Int 14b

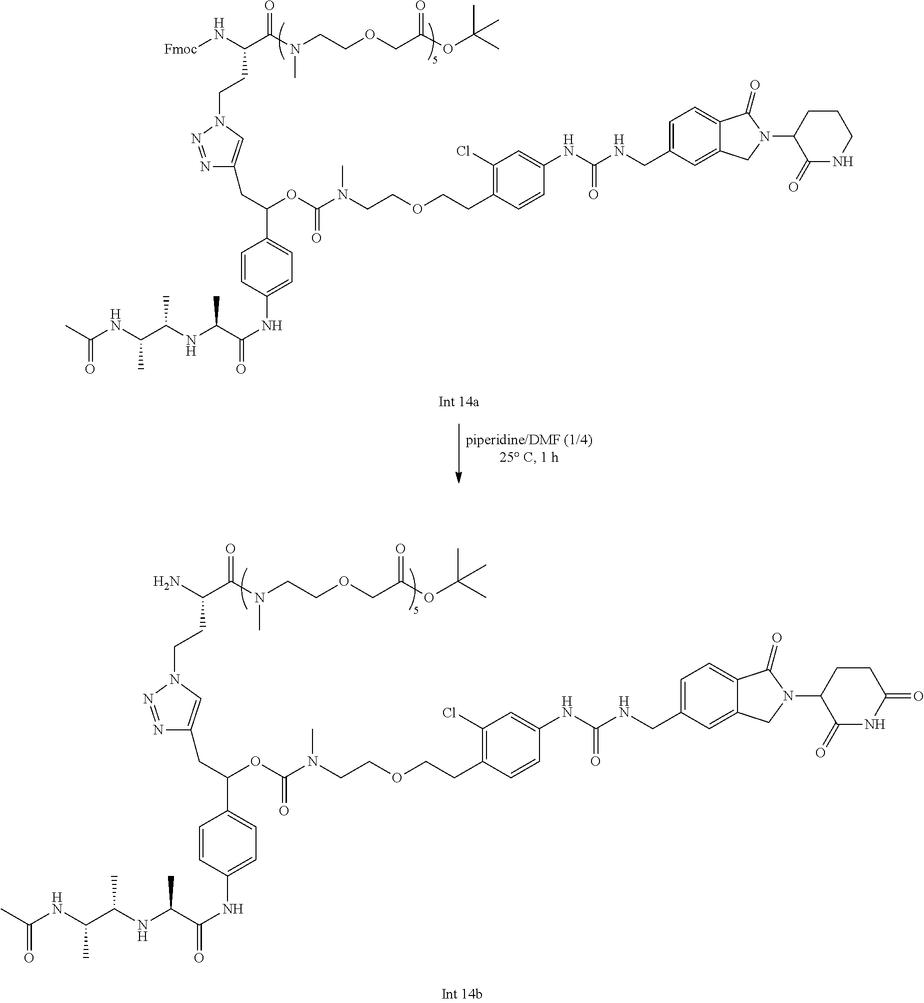

To a solution of tert-butyl (5S)-5-(2-(4-(2-(4-((S)-2-((S)-2-acetamidopropanamido)propanamido)phenyl)-2-(((2-(2-chloro-4-(3-((2-(2,6-dioxopiperidin-3-yl)-1-oxoisoindolin-5-yl)methyl)ureido)phenethoxy)ethyl) (methyl)carbamoyl)oxy)ethyl)-1H-1,2,3-triazol-1-yl)ethyl)-1-(9H-fluoren-9-yl)-7,13,19,25,31-pentamethyl-3,6,12,18,24,30-hexaoxo-2,10,16,22,28,34-hexaoxa-4,7,13,19,25,31-hexaazahexatriacontan-36-oate, Int 14a, (200 mg, 105 umol, 1.00 eq) in N-methyl pyrrolidone (1.50 mL) was added piperidine (0.30 mL). The mixture was stirred at 20° C. for 1 h. The reaction mixture was filtered to give filtrate. The filtrate was purified by reversed-phase HPLC (0.1% FA) to give tert-butyl (32S)-34-(4-(2-(4-((S)-2-((S)-2-acetamidopropanamido) propanamido)phenyl)-2-(((2-(2-chloro-4-(3-((2-(2,6-dioxopiperidin-3-yl)-1-oxoisoindolin-5-yl)methyl) ureido)phenethoxy)ethyl)(methyl)carbamoyl)oxy)ethyl)-1H-1,2,3-triazol-1-yl)-32-amino-6,12,18,24,30-pentamethyl-7,13,19,25,31-pentaoxo-3,9,15,21,27-pentaoxa-6,12,18,24,30-pentaazatetratriacontanoate, Int 14b, (100 mg, 59.7 umol, 57% yield) as a black solid. MS (ESI) m/z 838.3 [1/2 M+H]⁺

### Step C. Preparation of Int 14c

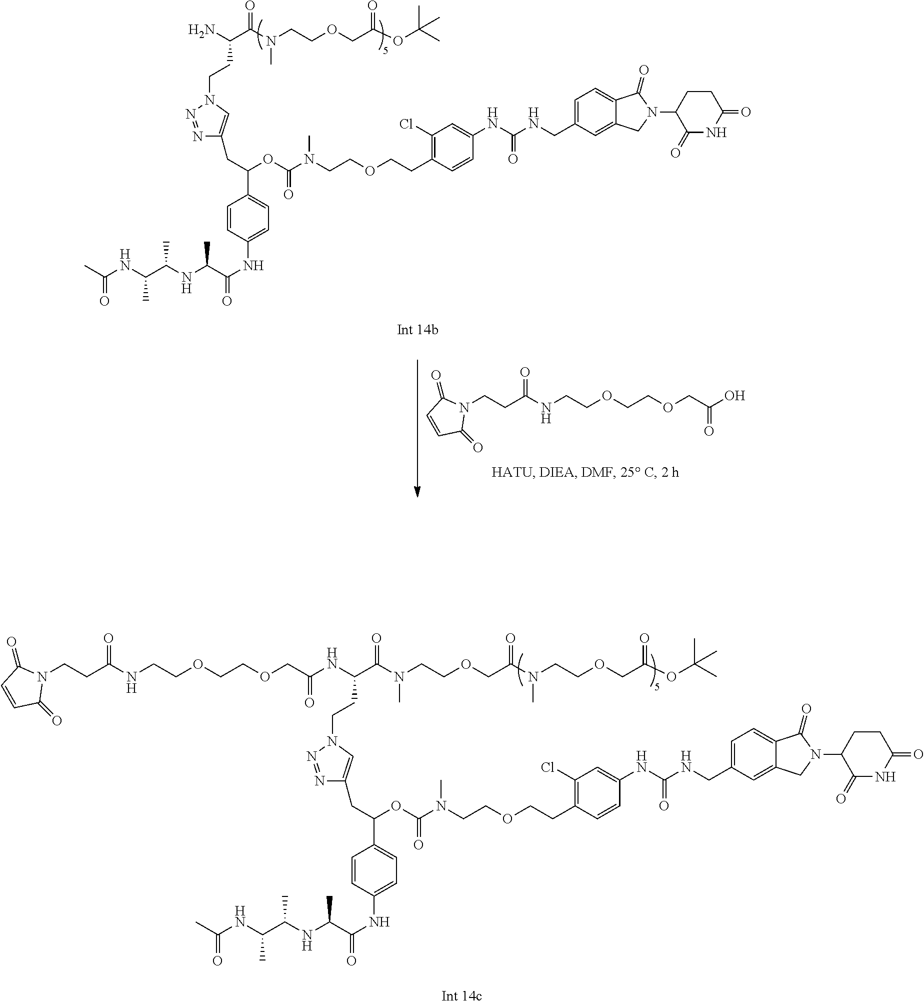

To a solution of 2-(2-(2-(3-(2,5-dioxo-2,5-dihydro-1H-pyrrol-1-yl)propanamido)ethoxy)ethoxy)acetic acid (22.5 mg, 71.6 umol, 1.20 eq) in dimethyl formamide (2.00 mL) was added tert-butyl (32S)-34-(4-(2-(4-((S)-2-((S)-2-acetamidopropanamido)propanamido)phenyl)-2-(((2-(2-chloro-4-(3-((2-(2,6-dioxopiperidin-3-yl)-1-oxoisoindolin-5-yl)methyl)ureido)phenethoxy)ethyl) (methyl)carbamoyl)oxy)ethyl)-1H-1,2,3-triazol-1-yl)-32-amino-6,12,18,24,30-pentamethyl-7,13,19,25,31-pentaoxo-3,9,15,21,27-pentaoxa-6,12,18,24,30-pentaazatetratriacontanoate, Int 14b, (100 mg, 59.7 umol, 1.00 eq), 0-(7-Azabenzotriazol-1-yl)-N,N,N′,N′-tetramethyluronium hexafluorophosphate (29.5 mg, 77.6 umol, 1.30 eq) and N,N-diisopropylethylamine (15.4 mg, 119 umol, 20.8 uL, 2.00 eq). The mixture was stirred at 25° C. for 2 h. The reaction mixture was filtered. The filtrate was purified by reversed-phase HPLC (0.1% FA) to give tert-butyl (32S)-32-(2-(4-(2-(4-((S)-2-((S)-2-acetamidopropanamido)propanamido)phenyl)-2-(((2-(2-chloro-4-(3-((2-(2,6-dioxopiperidin-3-yl)-1-oxoisoindolin-5-yl)methyl)ureido)phenethoxy)ethyl)(methyl)carbamoyl)oxy)ethyl)-1H-1,2,3-triazol-1-yl) ethyl)-45-(2,5-dioxo-2,5-dihydro-1H-pyrrol-1-yl)-6,12,18,24,30-pentamethyl-7,13,19,25,31,34,43-heptaoxo-3,9,15,21,27,36,39-heptaoxa-6,12,18,24,30,33,42-heptaazapentatetracontanoate, Int 14c (40.0 mg, 20.3 μmol, 34% yield) as a yellow solid. ¹H NMR (400 MHz, DMSO-d₆) δ=11.02-10.95 (m, 1H), 9.89-9.83 (m, 1H), 8.80-8.75 (m, 1H), 8.13 (d, J=7.6 Hz, 1H), 8.08 (d, J=7.6 Hz, 1H), 7.94-7.86 (m, 1H), 7.81-7.75 (m, 1H), 7.70-7.65 (m, 2H), 7.58-7.49 (m, 3H), 7.43 (d, J=7.6 Hz, 1H), 7.28-7.20 (m, 3H), 7.16-7.11 (m, 2H), 6.99 (s, 1H), 6.83-6.77 (m, 1H), 6.74-6.62 (m, 1H), 5.79-5.70 (m, 1H), 5.10 (dd, J=4.8, 13.6 Hz, 1H), 4.42-4.10 (m, 18H), 4.00-3.91 (m, 4H), 3.58-3.45 (m, 20H), 3.24-3.04 (m, 7H), 2.94-2.77 (m, 24H), 2.03-1.97 (m, 2H), 1.84 (s, 3H), 1.75 (s, 3H), 1.40 (s, 9H), 1.29 (br d, J=7.2 Hz, 3H), 1.19 (d, J=7.2 Hz, 3H). MS (ESI) m/z 986.5 [1/2 M+H]⁺

### Step E. Preparation of Compound IG-L-14

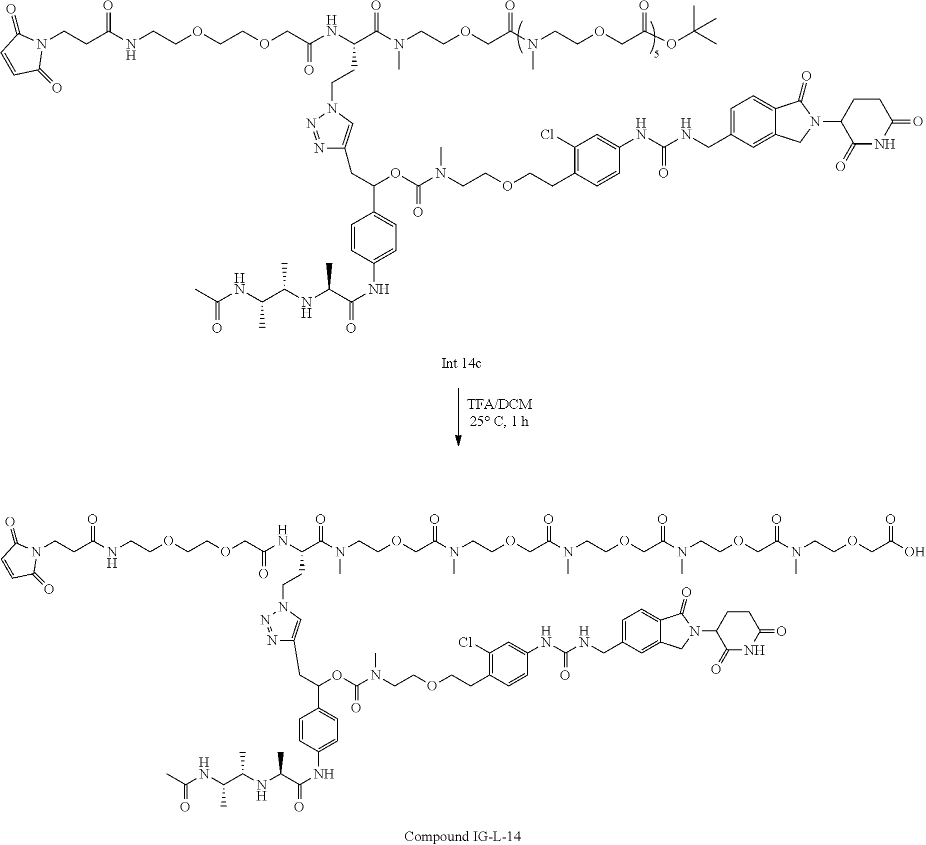

To a solution of tert-butyl (32S)-32-(2-(4-(2-(4-((S)-2-((S)-2-acetamidopropanamido)propanamido)phenyl)-2-(((2-(2-chloro-4-(3-((2-(2,6-dioxopiperidin-3-yl)-1-oxoisoindolin-5-yl)methyl)ureido)phenethoxy)ethyl) (methyl)carbamoyl)oxy)ethyl)-1H-1,2,3-triazol-1-yl)ethyl)-45-(2,5-dioxo-2,5-dihydro-1H-pyrrol-1-yl)-6,12,18,24,30-pentamethyl-7,13,19,25,31,34,43-heptaoxo-3,9,15,21,27,36,39-heptaoxa-6,12,18,24,30,33,42-heptaazapentatetracontanoate, Int 14c, (20.0 mg, 10.1 μmol, 1.00 eq) in dichloromethane (2.00 mL) was added trifluoroacetic acid (0.40 mL). The mixture was stirred at 25° C. for 1 h. The reaction mixture was concentrated under reduced pressure to give a residue. The residue was purified by prep-HPLC (column: Waters Xbridge 150*25 mm*5 um; mobile phase: [Water-ACN]; B %: 10%-40%, 9 min) to give (32S)-32-(2-(4-(2-(4-((S)-2-((S)-2-acetamidopropanamido)propanamido)phenyl)-2-(((2-(2-chloro-4-(3-((2-(2,6-dioxopiperidin-3-yl)-1-oxoisoindolin-5-yl)methyl)ureido)phenethoxy)ethyl)(methyl)carbamoyl)oxy)ethyl)-1H-1,2,3-triazol-1-yl)ethyl)-45-(2,5-dioxo-2,5-dihydro-1H-pyrrol-1-yl)-6,12,18,24,30-pentamethyl-7,13,19,25,31,34,43-heptaoxo-3,9,15,21,27,36,39-heptaoxa-6,12,18,24,30,33,42-heptaazapentatetracontanoic acid, Compound IG-L-14, (7.48 mg, 3.67 μmol, 36% yield, 94% purity) as a yellow solid. 1H NMR (400 MHz, DMSO-d₆) δ=9.91 (br s, 1H), 823-8.15 (m, 1H), 8.09 (d, J=7.2 Hz, 1H), 8.06-7.98 (m, 1H), 7.96-7.85 (m, 1H), 7.82-7.76 (m, 1H), 7.73-7.66 (m, 2H), 7.63-7.54 (m, 2H), 7.51 (s, 1H), 7.48-7.40 (m, 1H), 7.25 (br d, J=8.1 Hz, 2H), 7.21-7.10 (m, 2H), 6.99 (s, 1H), 5.76 (br t, J=6.4 Hz, 1H), 5.14-5.03 (m, 1H), 4.92-4.65 (m, 1H), 4.48-4.02 (m, 17H), 3.94 (br d, J=5.6 Hz, 2H), 3.85 (br s, 2H), 3.63-3.48 (m, 20H), 3.43 (br s, 12H), 3.23-3.06 (m, 7H), 2.94-2.87 (m, 9H), 2.84-2.79 (m, 9H), 2.74-2.67 (m, 2H), 2.64-2.54 (m, 2H), 2.41-2.34 (m, 2H), 2.32-2.11 (m, 2H), 2.06-1.96 (m, 2H), 1.85 (s, 3H), 1.30 (d, J=7.2 Hz, 3H), 1.20 (d, J=7.2 Hz, 3H). MS (ESI) m/z 958.5 [1/2 M+H]⁺

### Example L-35: Synthesis of 34-(2-(3-(((2S)-1-(((2S)-1-((4-(10-(3-(5-((3-(3-chloro-4-methylphenyl)ureido)methyl)-1-oxoisoindolin-2-yl)-2,6-dioxopiperidin-1-yl)-4-methoxy-3,8-dioxo-2,9-dioxa-4,7-diazadecyl)phenyl)amino)-1-oxopropan-2-yl)amino)-1-oxopropan-2-yl)amino)-3-oxopropyl)-5-(2,5-dioxo-2,5-dihydro-1H-pyrrol-1-yl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid (Compound IG-L-35)

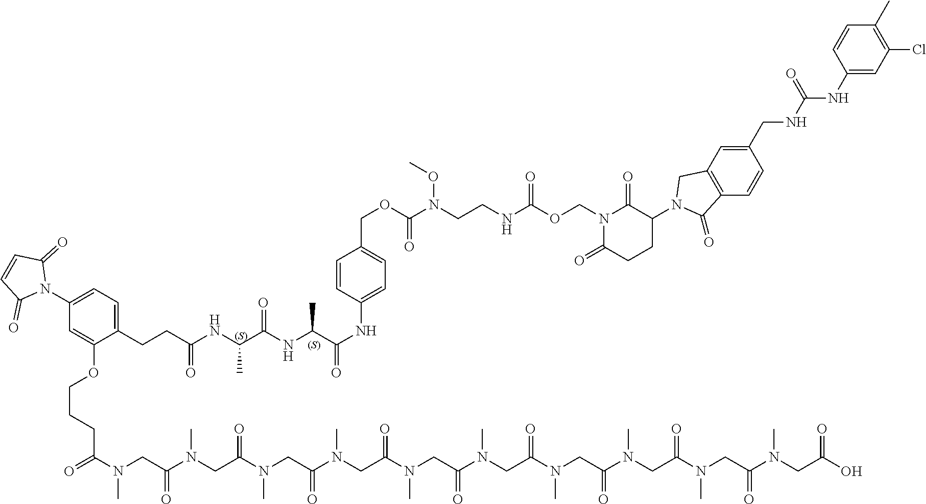

### Step A. Preparation of Int 35a

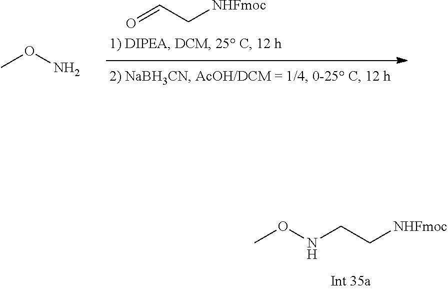

To a solution of O-methylhydroxylamine (891 mg, 10.7 mmol, 809 μL, 1.50 eq) in dichloromethane (20.0 mL) was added N,N-diisopropylethylamine (2.76 g, 21.3 mmol, 3.72 mL, 3.00 eq) and (9H-fluoren-9-yl)methyl (2-oxoethyl)carbamate (2.00 g, 7.11 mmol, 1.00 eq). The mixture was stirred at 25° C. for 12 h. Then the mixture was concentrated to afford a residue. The residue was dissolved in dichloromethane (20 mL) and acetic acid (5.00 mL), then sodium cyanoborohydride (670 mg, 10.7 mmol, 1.50 eq) was added to the mixture at 0° C. The mixture was stirred at 25° C. for 12 h. The mixture was filtered and the filtrate was concentrated to give a residue. The residue was purified by reversed-phase column (C18, 120 g; condition: water/acetonitrile=1/0-0/1, 0.1% formic acid) and lyophilized to afford (9H-fluoren-9-yl)methyl (2-(methoxyamino)ethyl)carbamate Int 35a (900 mg, 2.88 mmol, 40% yield) as a white solid. ¹H NMR (400 MHz, DMSO-d₆) δ=7.89 (d, J=7.6 Hz, 2H), 7.69 (d, J=7.6 Hz, 2H), 7.45-7.37 (m, 2H), 7.36-7.29 (m, 2H), 7.22 (br t, J=5.6 Hz, 1H), 6.57 (br t, J=5.6 Hz, 1H), 4.34-4.27 (m, 2H), 4.25-4.18 (m, 1H), 3.37 (s, 3H), 3.10 (q, J=6.4 Hz, 2H), 2.80 (q, J=6.4 Hz, 2H). MS (ESI) m/z 313.1 [M+H]⁺

### Step B. Preparation of Int 35b

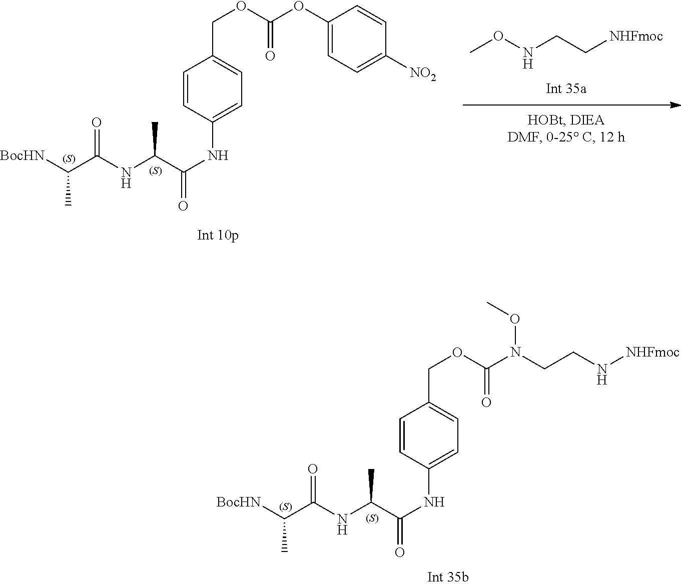

To a solution of (9H-fluoren-9-yl)methyl (2-(methoxyamino)ethyl)carbamate Int 35a (147 mg, 471 μmol, 1.00 eq) and tert-butyl ((S)-1-(((S)-1-((4-((((4-nitrophenoxy)carbonyl)oxy)methyl)phenyl)amino)-1-oxopropan-2-yl)amino)-1-oxopropan-2-yl)carbamate Int 10p (250 mg, 471 μmol, 1.00 eq) in N,N-dimethylformamide (2.00 mL) were added 1-hydroxybenzotriazole (127 mg, 942 gmol, 2.00 eq) and diisopropylethylamine (183 mg, 1.41 mmol, 246 μL, 3.00 eq) at 0° C. The mixture was stirred at 25° C. for 12 h. Six parallel reactions was combined and concentrated under reduced pressure to give a residue. The residue was purified by reversed-phase column (C18, 120 g; condition: water/acetonitrile=1/0-0/1, 0.1% formic acid) and lyophilized to afford 4-((S)-2-((S)-2-((tert-butoxycarbonyl)amino)propanamido)propanamido)benzyl (2-((((9H-fluoren-9-yl)methoxy)carbonyl)amino)ethyl)(methoxy)carbamate Int 35b (720 mg, 1.02 mmol, 36% yield) as a white solid. ¹H NMR (400 MHz, DMSO-d₆) δ=10.08-9.91 (m, 1H), 8.00 (d, J=7.2 Hz, 1H), 7.88 (d, J=7.6 Hz, 2H), 7.67 (d, J=7.6 Hz, 2H), 7.59 (d, J=8.4 Hz, 2H), 7.46-7.38 (m, 3H), 7.33-7.29 (m, 4H), 6.97 (br d, J=7.2 Hz, 1H), 5.03 (s, 2H), 4.45-4.34 (m, 1H), 4.28 (d, J=6.8 Hz, 2H), 4.23-4.16 (m, 1H), 3.99 (quin, J=7.2 Hz, 1H), 3.59 (s, 3H), 3.51 (br t, J=6.4 Hz, 2H), 3.16 (q, J=5.6 Hz, 2H), 1.37 (s, 9H), 1.29 (d, J=7.2 Hz, 3H), 1.18 (br d, J=7.2 Hz, 3H). MS (ESI) m/z 704.3 [M+H]⁺

### Step C. Preparation of Int 35c

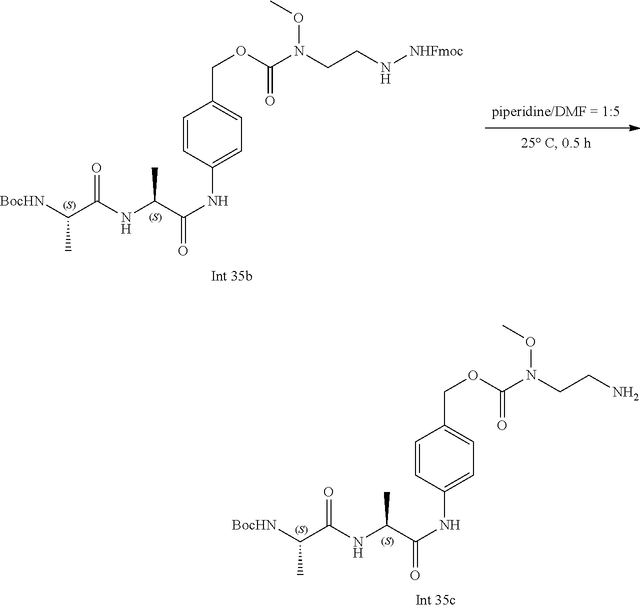

To a solution of 4-((S)-2-((S)-2-((tert-butoxycarbonyl)amino)propanamido)propanamido)benzyl (2-((((9H-fluoren-9-yl)methoxy)carbonyl)amino)ethyl)(methoxy)carbamate Int 35b (600 mg, 852 μmol, 1.00 eq) in N,N-dimethylformamide (1.00 mL) was added piperidine (1.03 g, 12.2 mmol, 1.20 mL, 14.3 eq). The mixture was stirred at 25° C. for 0.5 h. The mixture was adjusted with formic acid (50%) to pH=7 and concentrated to give a residue. The residue was purified by reversed-phase column (acetonitrile-water) and lyophilized to afford 4-((S)-2-((S)-2-((tert-butoxycarbonyl)amino)propanamido)propanamido)benzyl (2-aminoethyl)(methoxy)carbamate Int 35c (320 mg, 664 μmol, 78% yield) as a white solid. ¹H NMR (400 MHz, DMSO-d₆) δ=10.06 (s, 1H), 8.08 (d, J=7.2 Hz, 1H), 7.61 (d, J=8.4 Hz, 2H), 7.33 (d, J=8.4 Hz, 2H), 6.98 (br d, J=7.2 Hz, 1H), 5.07 (s, 2H), 4.41-4.36 (m, 1H), 4.03-3.95 (m, 1H), 3.61 (s, 3H), 3.57 (t, J=6.4 Hz, 2H), 2.83 (t, J=6.4 Hz, 2H), 1.37 (s, 9H), 130 (d, J=7.2 Hz, 3H), 1.18 (br d, J=7.2 Hz, 3H). MS (ESI) m/z 482.2 [M+H]⁺

### Step D. Preparation of Int 35d

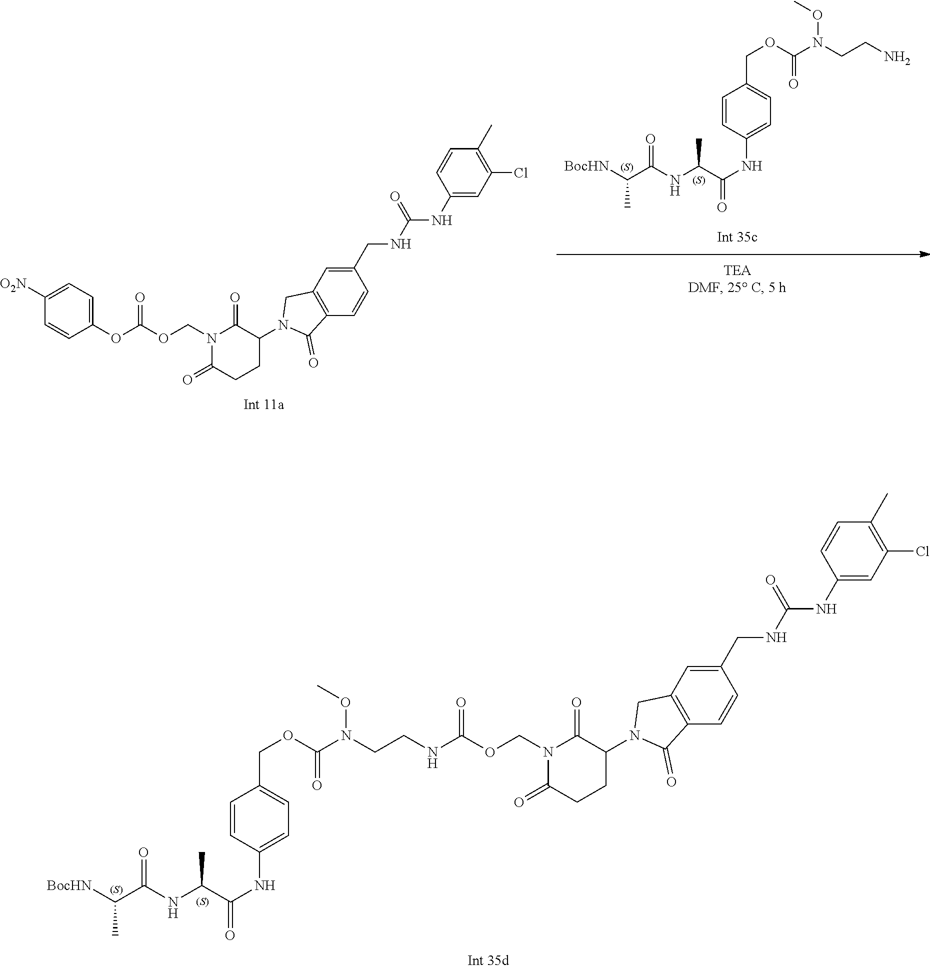

To a solution of 4-((S)-2-((S)-2-((tert-butoxycarbonyl)amino)propanamido)propanamido)benzyl (2-aminoethyl)(methoxy)carbamate Int 35c (363 mg, 755 gmol, 1.00 eq) in NN-dimethylformamide (4.00 mL) was added triethylamine (229 mg, 2.26 mmol, 315 μL, 3.00 eq) and (3-(5-((3-(3-chloro-4-methylphenyl)ureido)methyl)-1-oxoisoindolin-2-yl)-2,6-dioxopiperidin-1-yl)methyl (4-nitrophenyl) carbonate (480 mg, 758 μmol, 1.00 eq). The mixture was stirred at 25° C. for 5 h. The mixture was concentrated under reduced pressure to give a residue. The residue was purified by reversed-phase column (C18, 80 g; condition: water/acetonitrile=1/0-0/1, 0.1% formic acid) and lyophilized to afford 4-((S)-2-((S)-2-((tert-butoxycarbonyl)amino)propanamido)propanamido)benzyl (2-((((3-(5-((3-(3-chloro-4-methylphenyl) ureido)methyl)-1-oxoisoindolin-2-yl)-2,6-dioxopiperidin-1-yl)methoxy)carbonyl)amino)ethyl) (methoxy) carbamate Int 35d (510 mg, 521 μmol, 69% yield) as a white solid. ¹H NMR (400 MHz, DMSO-d₆) δ=9.99 (s, 1H), 8.75 (s, 1H), 8.00 (d, J 15=7.2 Hz, 1H), 7.70 (d, J=7.6 Hz, 1H), 7.66 (d, J=1.6 Hz, 1H), 7.58 (d, J=8.4 Hz, 2H), 7.52 (s, 1H), 7.48-7.38 (m, 2H), 7.31 (d, J=8.8 Hz, 2H), 7.20-7.15 (m, 1H), 7.15-7.10 (m, 1H), 6.97 (br d, J=7.2 Hz, 1H), 6.80 (t, J=6.0 Hz, 1H), 5.64-5.51 (m, 2H), 5.22 (br dd, J=5.2, 13.5 Hz, 1H), 5.05 (s, 2H), 4.49-4.25 (m, 5H), 4.03-3.93 (m, 1H), 3.60-3.56 (m, 3H), 3.53-3.45 (m, 2H), 3.19-3.04 (m, 3H), 2.88-2.77 (m, 1H), 2.38 (br dd, J=3.6, 14.1 Hz, 1H), 2.23 (s, 3H), 2.13-2.08 (m, 1H), 1.37 (s, 9H), 1.30 (d, J=7.2 Hz, 3H), 1.18 (br d, J=7.2 Hz, 3H). MS (ESI) m/z 978.5 [M+H]⁺

### Step E. Preparation of Int 35e

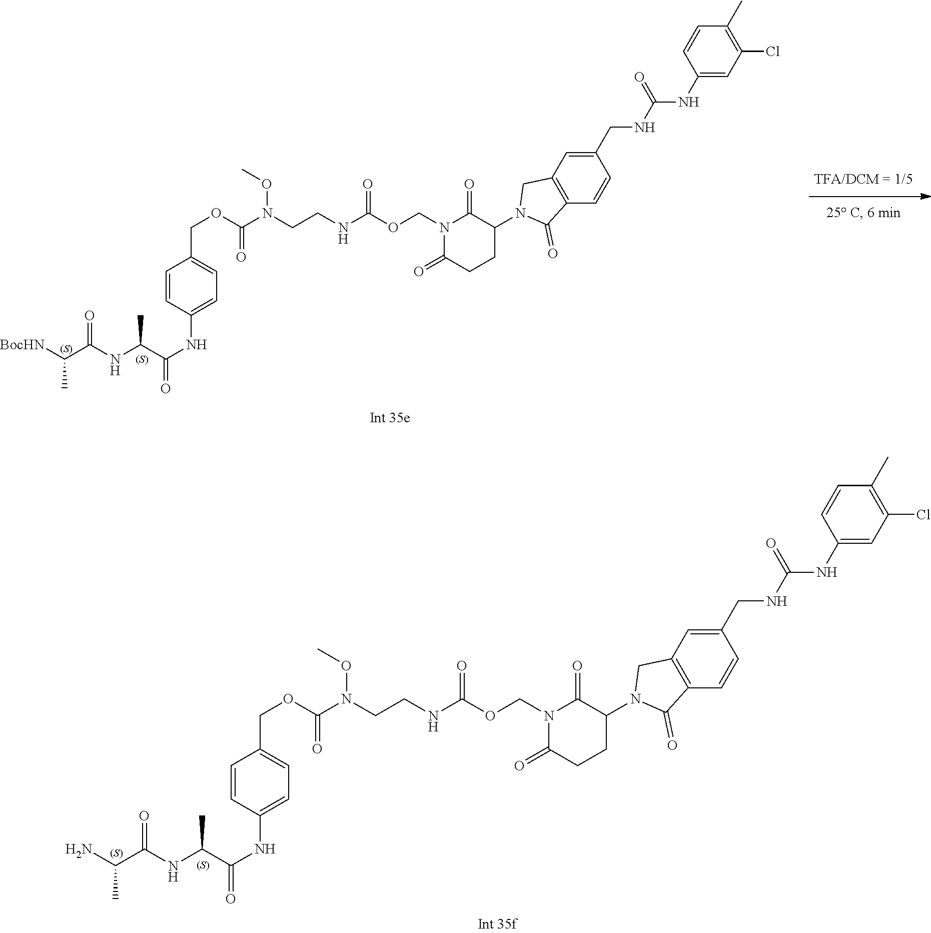

To a solution of 4-((S)-2-((S)-2-((tert-butoxycarbonyl)amino)propanamido)propanamido)benzyl (2-((((3-(5-((3-(3-chloro-4-methylphenyl)ureido)methyl)-1-oxoisoindolin-2-yl)-2,6-dioxopiperidin-1-yl)methoxy)carbonyl)amino)ethyl)(methoxy)carbamate Int 35d (510 mg, 521 μmol, 1.00 eq) in dichloromethane (5.00 mL) was added trifluoroacetic acid (1.54 g, 13.5 mmol, 1.00 mL, 25.8 eq). The mixture was stirred at 25° C. for 6 min. The mixture was concentrated under reduced pressure to give a residue. The residue was purified by reversed-phase column (C18, 12 g; condition: water/acetonitrile=1/0-0/1, 0.1% formic acid) and lyophilized to afford 4-((S)-2-((S)-2-aminopropanamido)propanamido)benzyl (2-((((3-(5-((3-(3-chloro-4-methylphenyl)ureido)methyl)-1-oxoisoindolin-2-yl)-2,6-dioxopiperidin-1-yl)methoxy)carbonyl)amino)ethyl)(methoxy)carbamate Int 35e (90.0 mg, 99.4 μmol, 19% yield, 97% purity) as a white solid. ¹H NMR (400 MHz, DMSO-d₆) δ=10.14 (s, 1H), 9.04-8.89 (m, 1H), 8.51-8.33 (m, 1H), 7.72-7.65 (m, 2H), 7.59 (d, J=8.4 Hz, 2H), 7.52 (s, 1H), 7.47-7.39 (m, 2H), 7.32 (d, J=8.4 Hz, 2H), 7.20-7.12 (m, 2H), 7.08-6.97 (m, 1H), 5.57 (q, J=9.2 Hz, 2H), 5.22 (br dd, J=5.2, 13.2 Hz, 1H), 5.05 (s, 2H), 4.56-4.18 (m, 6H), 3.58 (s, 3H), 3.49 (br s, 3H), 3.14-3.05 (m, 3H), 2.86-2.79 (m, 1H), 2.54 (br s, 1H), 2.47-2.35 (m, 1H), 2.22 (s, 3H), 2.13-1.97 (m, 1H), 1.32 (d, J=7.2 Hz, 3H), 1.25 (d, J=6.8 Hz, 3H). MS (ESI) m/z 878.4 [M+H]⁺

### Step F. Preparation of Compound IG-L-35

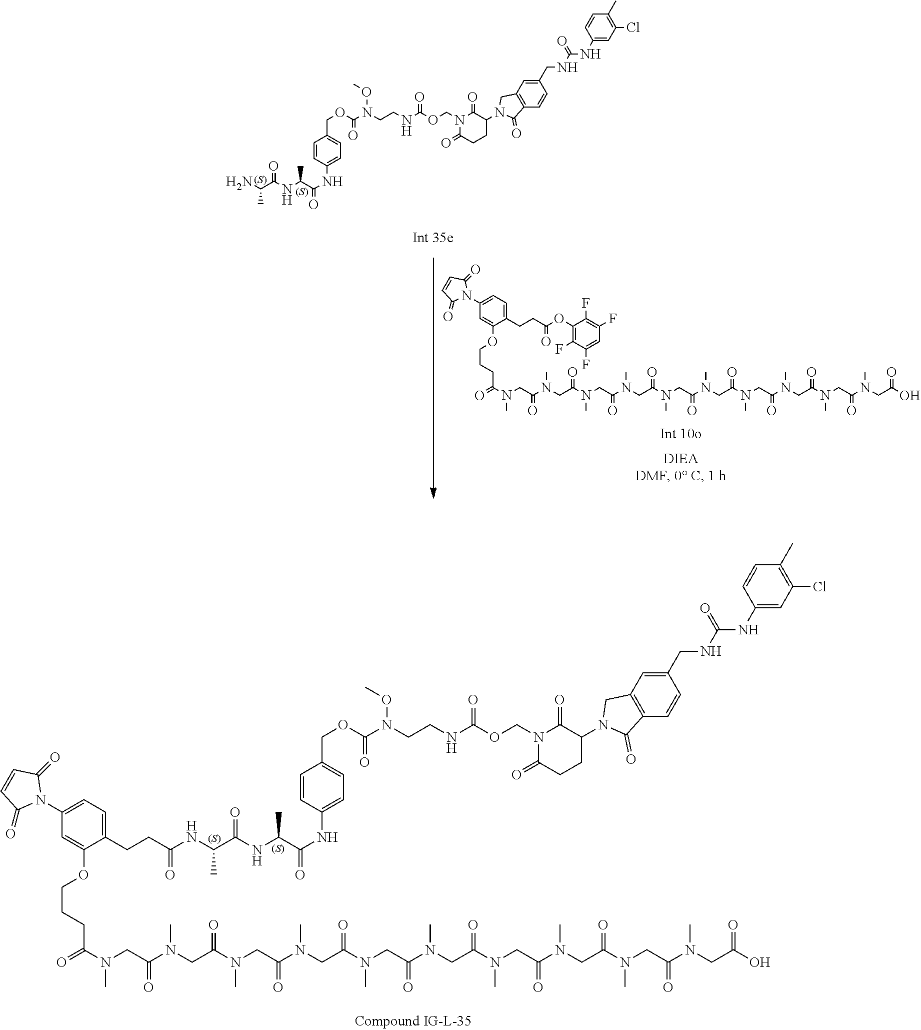

To a solution of 4-((S)-2-((S)-2-aminopropanamido)propanamido)benzyl (2-((((3-(5-((3-(3-chloro-4-methylphenyl)ureido)methyl)-1-oxoisoindolin-2-yl)-2,6-dioxopiperidin-1-yl)methoxy)carbonyl)amino) ethyl)(methoxy)carbamate Int 35e (20.0 mg, 22.8 gmol, 1.00 eq) and 34-(5-(2,5-dioxo-2,5-dihydro-1H-pyrrol-1-yl)-2-(3-oxo-3-(2,3,5,6-tetrafluorophenoxy)propyl)phenoxy)-3,6,9,12,15,18,21,24,27,30-decamethyl-4,7,10,13, 16,19,22,25,28,31-decaoxo-3,6,9,12,15,18,21,24,27,30-decaazatetratriacontanoic acid (24.7 mg, 20.5 μmol, 0.900 eq) in N,N-dimethylformamide (1.00 mL) was added diisopropylethylamine (8.83 mg, 68.3 μmol, 11.9 μL, 3.00 eq). The mixture was stirred at 0° C. for 1 h. The mixture was concentrated under reduced pressure to give a residue. The residue was purified by reversed-phase column (C18, 40 g; condition: water/acetonitrile=1/0-0/1, 0.1% formic acid), reversed-phase column (acetonitrile-water) and lyophilized to afford 34-(2-(3-(((2S)-1-(((2S)-1-((4-(10-(3-(5-((3-(3-chloro-4-methylphenyl)ureido)methyl)-1-oxoisoindolin-2-yl)-2,6-dioxopiperidin-1-yl)-4-methoxy-3,8-dioxo-2,9-dioxa-4,7-diazadecyl)phenyl)amino)-1-oxopropan-2-yl)amino)-1-oxopropan-2-yl)amino)-3-oxopropyl)-5-(2,5-dioxo-2,5-dihydro-1H-pyrrol-1-yl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid Compound IG-L-35 (4.34 mg, 2.19 gmol, 9% yield, 97% purity) as a yellow solid. ¹H NMR (400 MHz, DMSO-d₆) δ=10.06-9.90 (m, 1H), 8.34-8.00 (m, 2H), 7.72-7.65 (m, 2H), 7.62 (br d, J=8.4 Hz, 2H), 7.51 (br s, 1H), 7.47-7.38 (m, 2H), 7.31 (br d, J=8.4 Hz, 2H), 7.23-7.11 (m, 5H), 6.89 (br d, J=8.8 Hz, 1H), 6.77 (br t, J=4.8 Hz, 1H), 5.64-5.51 (m, 2H), 5.26-5.17 (m, 1H), 5.10-4.98 (m, 2H), 4.50-4.13 (m, 16H), 4.12-3.85 (m, 12H), 3.58 (s, 3H), 3.52-3.45 (m, 2H), 3.17-3.10 (m, 2H), 3.10-2.69 (m, 36H), 2.46-2.41 (m, 2H), 2.22 (s, 3H), 2.11-1.84 (m, 4H), 1.30 (br d, J=7.2 Hz, 3H), 1.19 (br d, J=7.2 Hz, 3H). MS (ESI) m/z 960.2 [M/2+2H]⁺

The compounds in Table 16 were prepared in a manner similar to that described for Compound IG-L-35 using the appropriate compound as starting material.

TABLE 16

| IG-L-No. | Structure | ¹H NMR/MS |
|---|---|---|
| IG-L-34 | 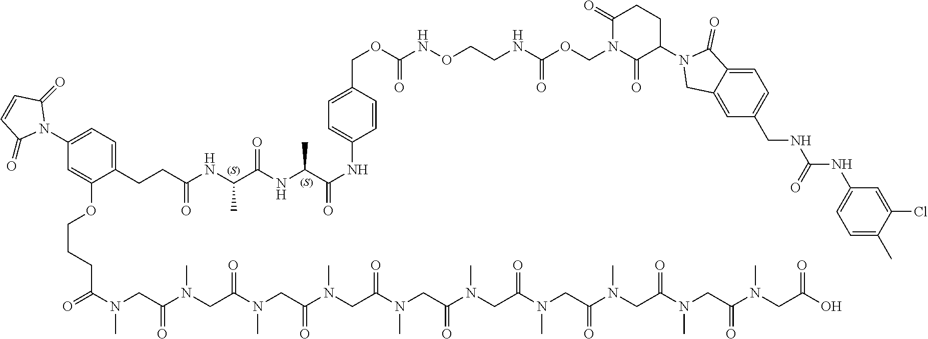 | ¹H NMR (400 MHz, DMSO-d₆) δ = 10.45- 10.30 (m, 1H), 10.04-9.91 (m, 1H), 8.31- 8.01 (m, 2H), 7.68 (br s, 2H), 7.65-7.59 (m, 2H), 7.52 (br s, 1H), 7.44 (br d, J = 7.6 Hz, 1H), 7.29 (br d, J = 7.6 Hz, 2H), 7.21 (br d, J = 8.0 Hz, 2H), 7.14 (br s, 4H), 6.90 (br d, J = 8.8 Hz, 1H), 6.82-6.74 (m, 1H), 5.62- 5.53 (m, 1H), 5.32-5.07 (m, 2H), 5.01 (br s, 2H), 4.48-4.16 (m, 16H), 4.12-3.85 (m, 12H), 3.74-3.69 (m, 2H), 3.44 (br dd, J = 1.2, 6.8 Hz, 2H), 3.20-3.14 (m, 2H), 2.97- 2.71 (m, 34H), 2.44 (br s, 1H), 2.42 (br s, 1H), 2.22 (br s, 3H), 2.09-2.02 (m, 1H), 1.99-1.89 (m, 2H), 1.30 (br d, J = 6.0 Hz, 3H), 1.19 (br d, J = 6.6 Hz, 3H). MS (ESI) m/z 952.8 [M/2 + H]⁺ |
| IG-L-55 | 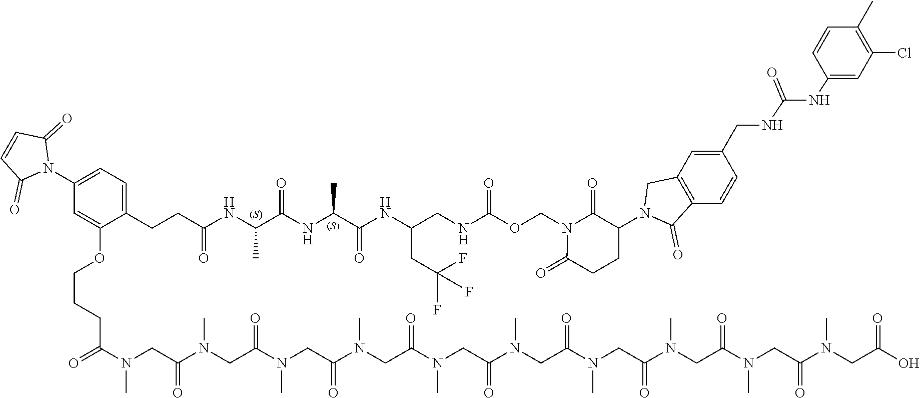 | ¹H NMR (400 MHz, DMSO-d₆) δ = 8.78 (br s, 1H), 8.13-8.00 (m, 1H), 7.98-7.88 (m, 1H), 7.86-7.76 (m, 1H), 7.75-7.62 (m, 2H), 7.52 (br s, 1H), 7.44 (br d, J = 8.0 Hz, 1H), 7.36 (br s, 1H), 7.23-7.10 (m, 5H), 6.95- 6.68 (m, 3H), 5.72-5.42 (m, 2H), 5.29-5.14 (m, 1H), 4.48-3.87 (m, 30H), 3.07-2.70 (m, 37H), 2.45-2.31 (m, 5H), 2.22 (s, 3H), 2.11- 1.88 (m, 3H), 1.17 (br d, J = 3.6 Hz, 6H). MS (ESI) m/z 911.5 [M/2 + 2H]⁺, 1821.9 [M + 2H]⁺ |
| IG-L-56 | 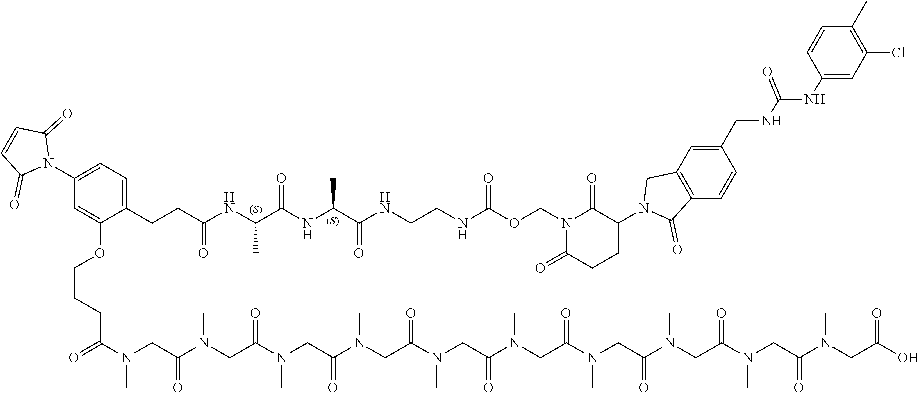 | ¹H NMR (400 MHz, DMSO-d₆) δ = 8.79 (br s, 1H), 8.09-7.99 (m, 1H), 7.90 (br d, J = 6.4 Hz, 1H), 7.86-7.77 (m, 1H), 7.70 (d, J = 7.6 Hz, 1H), 7.66 (d, J = 2.0 Hz, 1H), 7.52 (s, 1H), 7.44 (d, J = 7.6 Hz, 1H), 7.29-7.23 (m, 1H), 7.22-7.09 (m, 6H), 6.91-6.87 (m, 1H), 6.78 (br d, J = 7.6 Hz, 1H), 5.67-5.47 (m, 2H), 5.26-5.15 (m, 1H), 4.49-4.14 (m, 17H), 4.11-3.86 (m, 13H), 3.02-2.71 (m, 38H), 2.41 (br s, 2H), 2.22 (s, 3H), 2.09-2.03 (m, 1H), 1.98-1.88 (m, 2H), 1.17 (br t, J = 6.4 Hz, 6H). MS (ESI) m/z 1739.7 [M + 2H]⁺ |
| IG-L-60 | 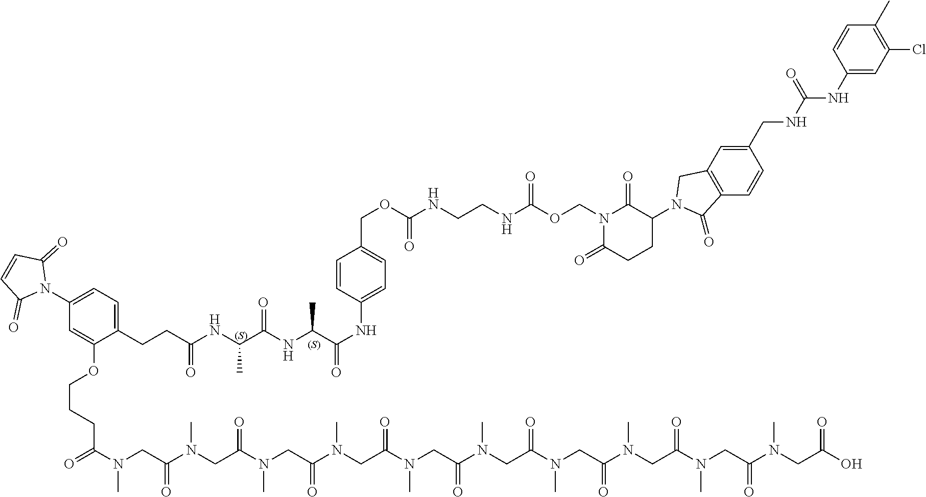 | ¹H NMR (400 MHz, DMSO-d₆) δ = 12.76 (br s, 1H), 9.93 (br s, 1H), 8.82 (s, 1H), 8.23-8.02 (m, 2H), 7.74-7.65 (m, 2H), 7.60 (br d, J = 7.6 Hz, 2H), 7.52 (s, 1H), 7.44 (d, J = 8.0 Hz, 1H), 7.34-7.12 (m, 9H), 6.93- 6.76 (m, 3H), 5.65-5.50 (m, 2H), 5.23 (br dd, J = 5.2, 13.4 Hz, 1H), 4.92 (s, 2H), 4.47-3.90 (m, 29H), 3.09-2.72 (m, 40H), 2.45-2.39 (m, 2H), 2.22 (s, 3H), 2.08-1.91 (m, 3H), 1.30 (br d, J = 7.2 Hz, 3H), 1.19 (br d, J = 7.2 Hz, 3H). MS (ESI) m/z 1889.7 [M+3H]+ |
| IG-L-61 | 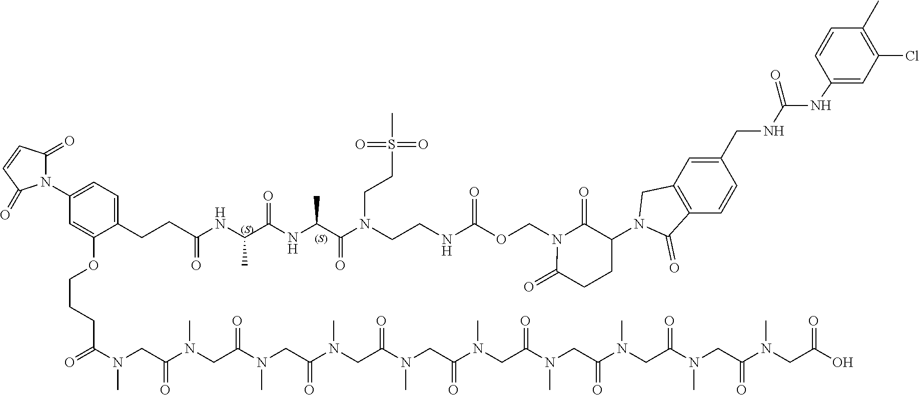 | ¹H NMR (400 MHz, DMSO-d₆) δ = 9.09- 8.85 (m, 1H), 8.26-8.08 (m, 1H), 8.03-7.95 (m, 1H), 7.71-7.66 (m, 2H), 7.55-7.41 (m, 3H), 7.31 (br d, J = 14.8 Hz, 1H), 7.20-7.13 (m, 5H), 6.89 (br d, J = 8.8 Hz, 1H), 6.78 (br d, J = 7.2 Hz, 1H), 5.65-5.51 (m, 2H), 5.27-5.17 (m, 1H), 4.61-4.54 (m, 1H), 4.50-4.16 (m, 17H), 4.10-3.88 (m, 13H), 3.74-3.62 (m, 2H), 3.57-3.52 (m, 2H), 3.02-2.71 (m, 40H), 2.42-2.31 (m, 3H), 2.22 (s, 3H), 2.10-1.99 (m, 1H), 1.99-1.86 (m, 2H), 1.19-1.10 (m, 6H). MS (ESI) m/z 1845.5 [M + 2H]⁺ |

### Example L-36: Synthesis of 34-(6-((6S,9S)-1-((3-chloro-4-methylphenyl)amino)-2-((2-(2,6-dioxopiperidin-3-yl)-1-oxoisoindolin-5-yl)methyl)-6,9-dimethyl-1,5,8,11-tetraoxo-2,4,7,10-tetraazatridecan-13-yl)-3-(2,5-dioxo-2,5-dihydro-1H-pyrrol-1-yl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, IG-L-36

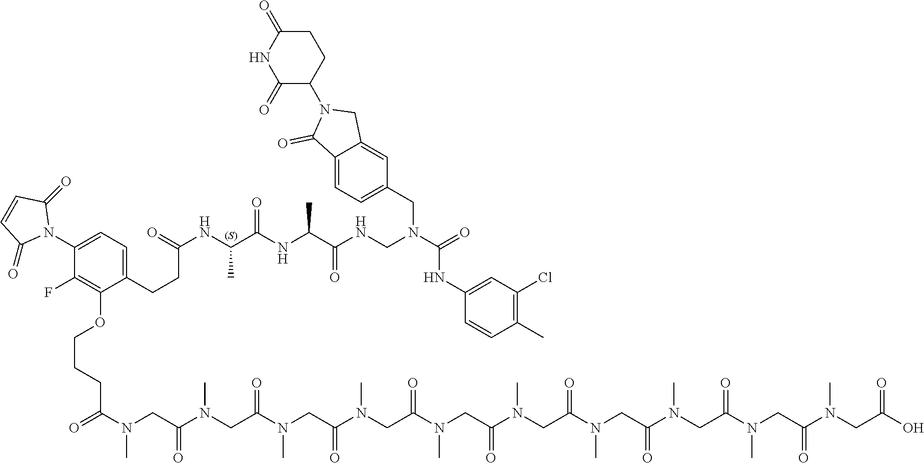

### Step A. Preparation of Int 36a

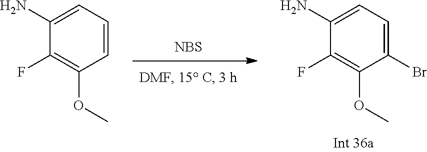

To a solution of 2-fluoro-3-methoxyaniline (11.0 g, 77.9 mmol, 1.00 equiv) in N,N-dimethyl formamide (40.0 mL) was added a solution of 1-bromopyrrolidine-2, 5-dione (13.9 g, 78.0 mmol, 1.00 equiv) dropwise. The mixture was stirred at 15° C. for 3 h. The mixture was diluted with brine (300 mL) and extracted with ethyl acetate (3×300 mL). The organic layers were collected and dried over anhydrous sodium sulfate. After filtration, the filtrate was concentrated under reduced pressure to give a crude product, which was purified by column chromatography on silica gel (SiO₂, petroleum ether/ethyl acetate=1/0 to 3/1) and concentrated to afford 4-bromo-2-fluoro-3-methoxyaniline, Int. 36a, (26.0 g, 118 mmol, 75% yield) as yellow oil. ¹H NMR (400 MHz, DMSO-d₆) δ=7.03 (dd, J=8.8, 1.2 Hz, 1H), 6.47 (t, J=8.8 Hz, 1H), 5.38 (s, 2H), 3.86-3.78 (m, 3H).

### Step B. Preparation of Int 36b

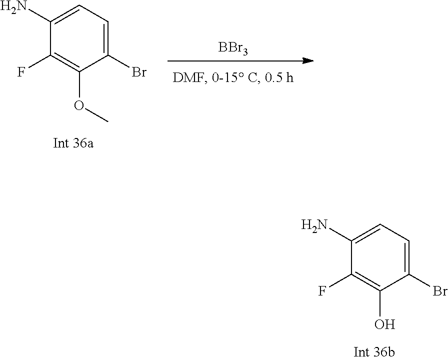

To a solution of 4-bromo-2-fluoro-3-methoxyaniline, Int. 36a, (10.0 g, 45.5 mmol, 1.00 equiv) in dichloromethane (100 mL) was added borontribromide (171 g, 682 mmol, 65.7 mL, 15.0 equiv) at 0° C. The mixture was stirred at 15° C. for 0.5 h. The reaction mixture was quenched by addition water (300 mL) at 0° C. After filtration, the filtrate was extracted with ethyl acetate (3×200 mL). The organic layers were collected and dried over anhydrous sodium sulfate. After filtration, the filtrate was concentrated under reduced pressure to afford 3-amino-6-bromo-2-fluorophenol, Int. 36b, (22.5 g, crude) as yellow oil. ¹H NMR (400 MHz, DMSO-d₆) δ=9.95-9.35 (m, 1H), 6.90 (dd, J=8.8, 1.6 Hz, 1H), 6.20-6.16 (m, 1H), 5.15 (br s, 2H). MS (ESI) m/z 205.9 [M+H]⁺

### Step C. Preparation of Int 36c

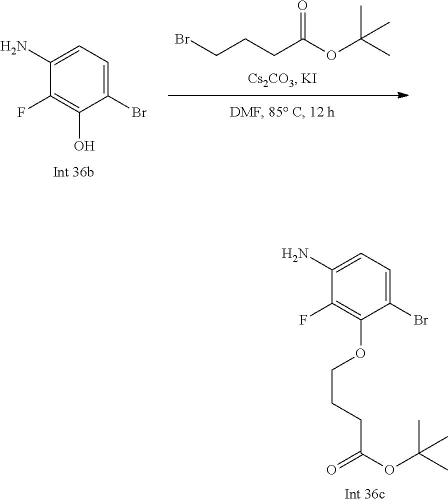

To a solution of 3-amino-6-bromo-2-fluorophenol, Int. 36b, (16.0 g, 77.7 mmol, 1.00 equiv), cesium carbonate (50.6 g, 155 mmol, 2.00 equiv) and potassium iodide (1.29 g, 7.77 mmol, 0.100 equiv) in N, N-dimethyl formamide (50.0 mL) was added tert-butyl 4-bromobutanoate (19.1 g, 85.4 mmol, 1.10 equiv). The mixture was stirred at 85° C. for 12 h. The mixture was diluted with brine (300 mL) and extracted with ethyl acetate (3×200 mL). The organic layers were collected and dried over anhydrous sodium sulfate. After filtration, the filtrate was concentrated under reduced pressure to give a crude product, which was purified by column chromatography on silica gel (SiO₂, petroleum ether/ethyl acetate=1/0 to 3/1) and concentrated to afford tert-butyl 4-(3-amino-6-bromo-2-fluorophenoxy)butanoate, Int. 36c, (17.0 g, 48.8 mmol, 62% yield) as brown oil. ¹H NMR (400 MHz, DMSO-d₆) δ=7.03 (dd, J=8.8, 1.6 Hz, 1H), 6.46 (t, J=8.8 Hz, 1H), 5.36 (s, 2H), 3.98-3.93 (m, 2H), 2.43 (t, J=7.2 Hz, 2H), 1.91 (br t, J=6.8 Hz, 2H), 1.40 (s, 9H).

### Step D. Preparation of Int 36d

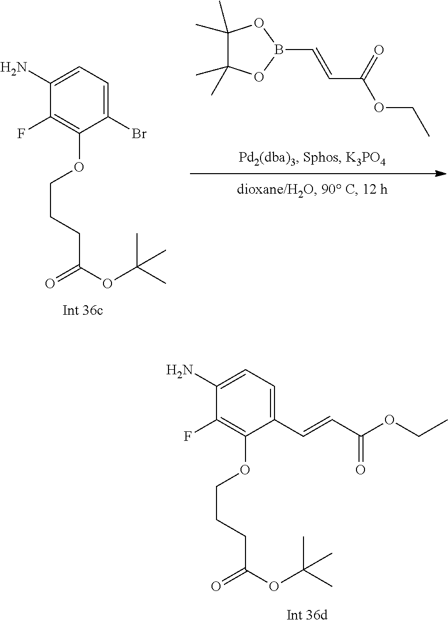 [SMILES: CC(C)(C)OC(=O)CCCOc1c(Br)ccc(N)c1F ; CCOC(=O)/C=C/B1OC(C)(C)C(C)(C)O1 ; CCOC(=O)/C=C/c1ccc(N)c(F)c1OCCCC(=O)OC(C)(C)C]

To a solution of tert-butyl 4-(3-amino-6-bromo-2-fluorophenoxy)butanoate, Int. 36c, (12.0 g, 34.5 mmol, 1.00 equiv) and ethyl (E)-3-(4,4,5,5-tetramethyl-1,3,2-dioxaborolan-2-yl)acrylate (19.5 g, 86.2 mmol, 2.50 equiv) in dioxane (100 mL) and water (10.0 mL) was added 2-dicyclohexylphosphino-2,6-dimethoxybiphenyl (1.41 g, 3.45 mmol, 0.100 equity), potassium phosphate (11.0 g, 51.7 mmol, 1.50 equiv) and bis(dibenzylideneacetone)-palladium(O) (1.58 g, 1.72 mmol, 0.050 equiv.). The mixture was stirred at 90° C. for 12 h under nitrogen atmosphere. The mixture was concentrated under reduced pressure to give a crude product, which was purified by column chromatography on silica gel (SiO₂, petroleum ether/ethyl acetate=1/0 to 3/1) and concentrated to afford tert-butyl (E)-4-(3-amino-6-(3-ethoxy-3-oxoprop-1-en-1-yl)-2-fluorophenoxy)butanoate, Int. 36d, (11.0 g, 30.0 mmol, 86% yield) as yellow solid. ¹H NMR (400 MHz, DMSO-d₆) δ=7.70 (d, J=16.0 Hz, 1H), 7.30 (dd, J=8.8, 1.2 Hz, 1H), 6.50 (t, J=8.4 Hz, 1H), 6.32 (d, J=16.0 Hz, 1H), 5.87 (s, 2H), 4.14 (q, J=7.2 Hz, 2H), 4.01 (t, J=6.4 Hz, 2H), 2.40 (t, J=7.2 Hz, 2H), 1.92 (quin, J=6.8 Hz, 2H), 1.38 (s, 9H), 1.25-1.22 (m, 3H).

### Step E. Preparation of Int 36e

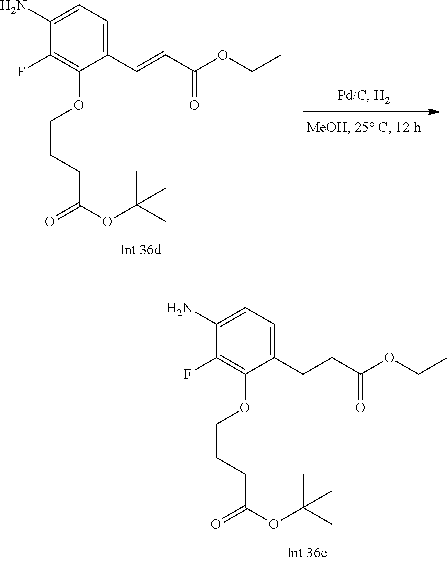 [SMILES: CCOC(=O)/C=C/c1ccc(N)c(F)c1OCCCC(=O)OC(C)(C)C ; CCOC(=O)CCc1ccc(N)c(F)c1OCCCC(=O)OC(C)(C)C]

A mixture of tert-butyl (E)-4-(3-amino-6-(3-ethoxy-3-oxoprop-1-en-1-yl)-2-fluorophenoxy)butanoate, Int. 36d, (5.00 g, 13.6 mmol, 1.00 equiv) in methanol (100 mL) was added palladium on activated carbon (5.00 g, 10% purity). The mixture was stirred at 25° C. for 12 h under hydrogen (50 Psi) atmosphere. The reaction mixture was filtered concentrated under reduced pressure to afford tert-butyl 4-(3-amino-6-(3-ethoxy-3-oxopropyl)-2-fluorophenoxy)butanoate, Int. 36e, (3.40 g, crude) as black oil. ¹H NMR (400 MHz, DMSO-d₆) δ=6.64 (dd, J=8.4, 1.6 Hz, 1H), 6.39 (t, J=8.4 Hz, 1H), 5.01-4.91 (m, 2H), 4.03 (q, J=6.8 Hz, 2H), 3.93 (t, J=6.8 Hz, 2H), 2.74-2.64 (m, 2H), 2.47-2.44 (m, 2H), 2.39 (t, J=6.8 Hz, 2H), 1.90 (quin, J=6.8 Hz, 2H), 1.40 (s, 9H), 1.15 (t, J=6.8 Hz, 3H).

### Step F. Preparation of Int 36f

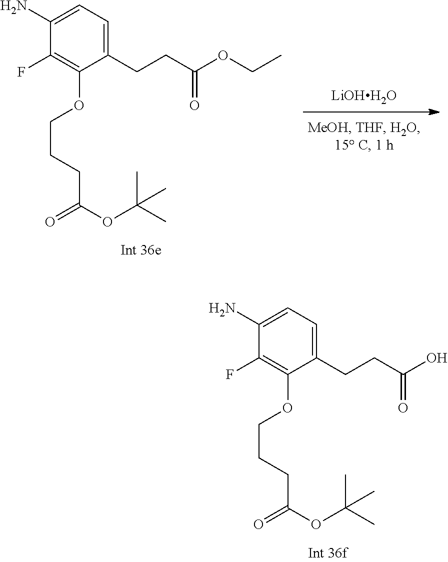 [SMILES: CCOC(=O)CCc1ccc(N)c(F)c1OCCCC(=O)OC(C)(C)C ; "LiOH·H2O" ; CC(C)(C)OC(=O)CCCOc1c(CCC(=O)O)ccc(N)c1F]

To a solution of tert-butyl 4-(3-amino-6-(3-ethoxy-3-oxopropyl)-2-fluorophenoxy)butanoate, Int. 36e, (5.00 g, 13.5 mmol, 1.00 equiv) in methanol (10.0 mL), tetrahydrofuran (10.0 mL) and water (10.0 mL) was added lithium hydroxide monohydrate (852 mg, 20.3 mmol, 1.50 equiv). The mixture was stirred at 15° C. for 1 h. The mixture was quenched with hydrochloric acid (1M) until pH=7. The mixture was concentrated to remove tetrahydrofuran and methanol. The mixture was diluted with water (150 mL) and extracted with ethyl acetate (3×150 mL). The organic layers were collected and dried over anhydrous sodium sulfate. After filtration, the filtrate was concentrated under reduced pressure to afford 3-(4-amino-2-(4-(tert-butoxy)-4-oxobutoxy)-3-fluorophenyl)propanoic acid, Int. 36f, (4.00 g, crude) as yellow oil. ¹H NMR (400 MHz, DMSO-d₆) δ=12.45-11.77 (m, 1H), 6.65 (dd, J=8.4, 1.2 Hz, 1H), 6.39 (t, J=8.4 Hz, 1H), 4.96 (s, 2H), 3.93 (t, J=6.4 Hz, 2H), 2.66 (t, J=7.2 Hz, 2H), 2.41-2.36 (m, 4H), 1.90 (quin, J=6.8 Hz, 2H), 1.40 (s, 9H).

### Step G. Preparation of Int 36g

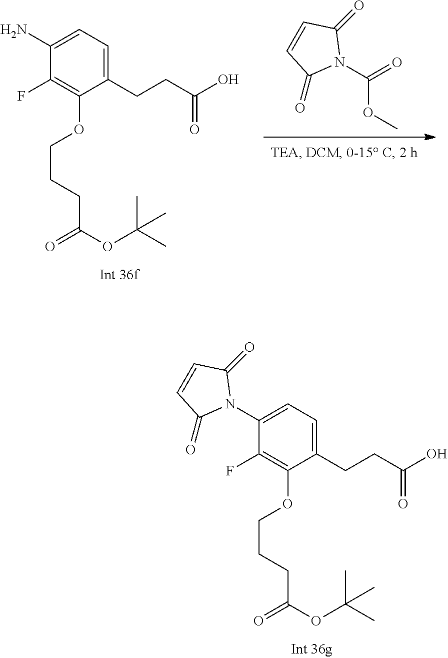 [SMILES: CC(C)(C)OC(=O)CCCOc1c(CCC(=O)O)ccc(N)c1F ; COC(=O)N1C(=O)C=CC1=O ; CC(C)(C)OC(=O)CCCOc1c(CCC(=O)O)ccc(N2C(=O)C=CC2=O)c1F]

To a solution of 3-(4-amino-2-(4-(tert-butoxy)-4-oxobutoxy)-3-fluorophenyl)propanoic acid, Int. 36f, (4.00 g, 11.7 mmol, 1.00 equiv) in dichloromethane (50.0 mL) was added triethylamine (3.56 g, 35.2 mmol, 4.89 mL, 3.00 equiv) and methyl 2,5-dioxo-2,5-dihydro-1H-pyrrole-1-carboxylate (2.00 g, 12.9 mmol, 2.00 mL, 1.10 equiv) at 0° C. The mixture was stirred at 15° C. for 2 h. The mixture was diluted with water (100 mL) and extracted with dichloromethane (3×80 mL). The organic layers were collected and dried over anhydrous sodium sulfate. After filtration, the filtrate was concentrated under reduced pressure to afford 3-(2-(4-(tert-butoxy)-4-oxobutoxy)-4-(2,5-dioxo-2,5-dihydro-1H-pyrrol-1-yl)-3-fluorophenyl)propanoic acid, Int. 36g, (4.50 g, crude) as brown oil. ¹H NMR (400 MHz, DMSO-d₆) δ=7.25 (s, 2H), 7.16 (d, J=8.8 Hz, 1H), 7.08-7.03 (m, 1H), 4.01 (br t, J=6.4 Hz, 2H), 2.87 (br t, J=7.6 Hz, 2H), 2.52-2.51 (m, 2H), 2.42-2.36 (m, 4H), 1.39 (s, 9H).

### Step H. Preparation of Int 36h

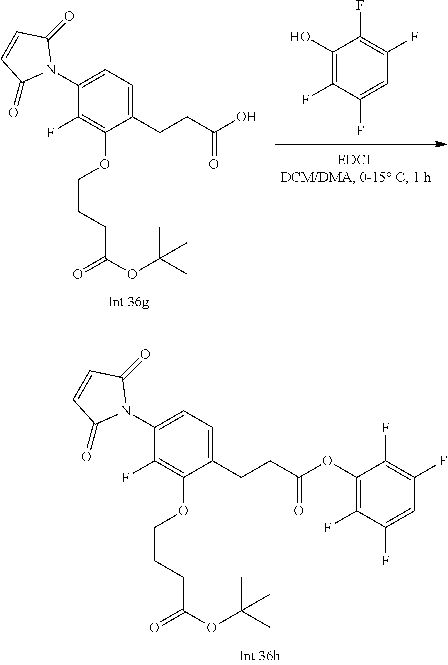 [SMILES: CC(C)(C)OC(=O)CCCOc1c(CCC(=O)O)ccc(N2C(=O)C=CC2=O)c1F ; Oc1c(F)c(F)cc(F)c1F ; CC(C)(C)OC(=O)CCCOc1c(CCC(=O)Oc2c(F)c(F)cc(F)c2F)ccc(N2C(=O)C=CC2=O)c1F]

To a solution of 3-(2-(4-(tert-butoxy)-4-oxobutoxy)-4-(2,5-dioxo-2,5-dihydro-1H-pyrrol-1-yl)-3-fluorophenyl)propanoic acid, Int. 36g, (4.50 g, 10.7 mmol, 1.00 equiv) and 2,3,5,6-tetrafluorophenol (3.55 g, 21.4 mmol, 2.00 equiv) in dichloromethane (50.0 mL) and N,N-dimethylacetamide (5.00 mL) was added 1-(3-dimethylaminopropyl)-3-ethylcarbodiimide hydrochloride (4.09 g, 21.4 mmol, 2.00 equiv) at 0° C. The mixture was stirred at 15° C. for 1 h. The mixture was concentrated under reduced pressure to give a crude product, which was purified by reversed-phase HPLC (column: spherical C18, 20-45 um, 100 Å, SW 330, mobile phase: [0.1% formic acid-acetonitrile]; B %: 85%-100%, 60 min). The desired fraction collected and lyophilized to afford tert-butyl 4-(3-(2, 5-dioxo-2,5-dihydro-1H-pyrrol-1-yl)-2-fluoro-6-(3-oxo-3-(2,3,5,6-tetrafluorophenoxy)propyl)phenoxy)butanoate, Int. 36h, (3.00 g, 5.27 mmol, 49% yield) as a brown solid. ¹H NMR (400 MHz, DMSO-d₆) δ=7.97-7.91 (m, 1H), 7.26 (s, 2H), 7.24 (s, 1H), 7.13-7.08 (m, 1H), 4.06 (t, J=6.4 Hz, 2H), 3.18-3.12 (m, 2H), 3.10-3.04 (m, 2H), 2.43-2.39 (m, 2H), 1.97-1.92 (m, 2H), 1.37 (s, 9H). MS (ESI) m/z 587.1 [M+H₂O]⁺

### Step I. Preparation of Int 36i

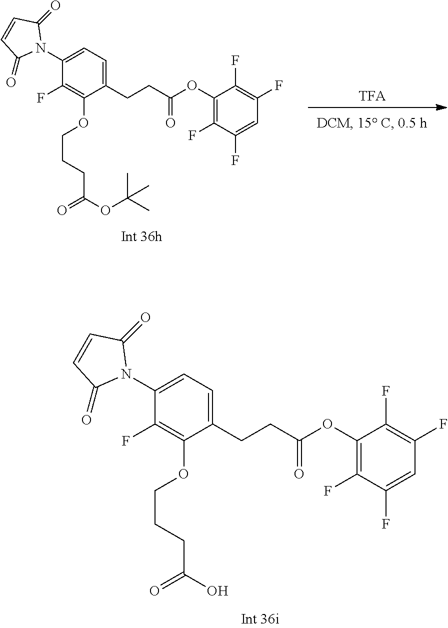 [SMILES: CC(C)(C)OC(=O)CCCOc1c(CCC(=O)Oc2c(F)c(F)cc(F)c2F)ccc(N2C(=O)C=CC2=O)c1F ; "TFA" ; O=C(O)CCCOc1c(CCC(=O)Oc2c(F)c(F)cc(F)c2F)ccc(N2C(=O)C=CC2=O)c1F]

To a solution of tert-butyl 4-(3-(2,5-dioxo-2,5-dihydro-1H-pyrrol-1-yl)-2-fluoro-6-(3-oxo-3-(2,3,5,6-tetrafluorophenoxy)propyl)phenoxy)butanoate, Int. 36h, (1.00 g, 1.76 mmol, 1.00 equiv) in dichloromethane (15.0 mL) was added trifluoroacetic acid (76.8 g, 673 mmol, 50.0 mL, 383 equiv). The mixture was stirred at 15° C. for 0.5 h. The reaction mixture was lyophilized to give a crude product, which was triturated with methyl tert-butyl ether (10 mL) at 25° C. Then filtered, the filter cake was dried in vacuum to afford 4-(3-(2,5-dioxo-2,5-dihydro-1H-pyrrol-1-yl)-2-fluoro-6-(3-oxo-3-(2,3,5,6-tetrafluorophenoxy)propyl)phenoxy)butanoic acid, Int. 36i, (300 mg, 584 μmol, 33% yield) as a pink solid. ¹H NMR (400 MHz, DMSO-d₆) δ=13.15-11.06 (m, 1H), 7.94 (tt, J=7.6, 10.8 Hz, 1H), 7.26 (s, 2H), 7.24 (s, 1H), 7.15-7.06 (m, 1H), 4.08 (t, J=6.0 Hz, 2H), 3.23-3.13 (m, 2H), 3.11-3.02 (m, 2H), 2.42 (t, J=7.4 Hz, 2H), 1.96 (quin, J=6.8 Hz, 2H).

### Step J. Preparation of Int 36j

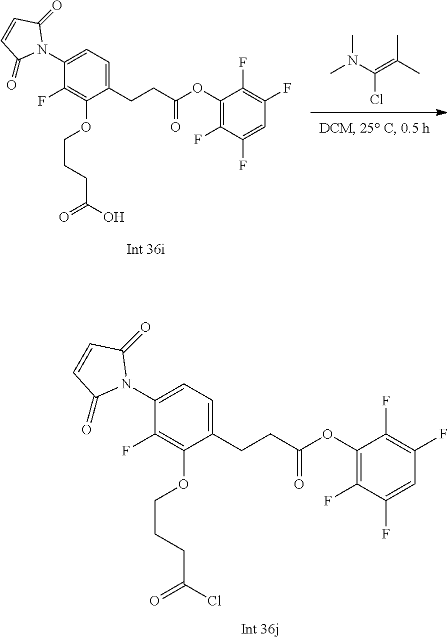

To a solution of 4-(3-(2,5-dioxo-2,5-dihydro-1H-pyrrol-1-yl)-2-fluoro-6-(3-oxo-3-(2,3,5,6-tetrafluorophenoxy)propyl)phenoxy)butanoic acid, Int. 36i, (100 mg, 195 μmol, 1.00 equiv) in dichloromethane (3.00 mL) was added 1-chloro-N,N,2-trimethylprop-1-en-1-amine, Int. 36j, (52.1 mg, 390 μmol, 51.5 μL, 2.00 equiv). The mixture was stirred at 25° C. for 0.5 h. The reaction mixture was used in next step directly.

### Step K. Preparation of Int 36k

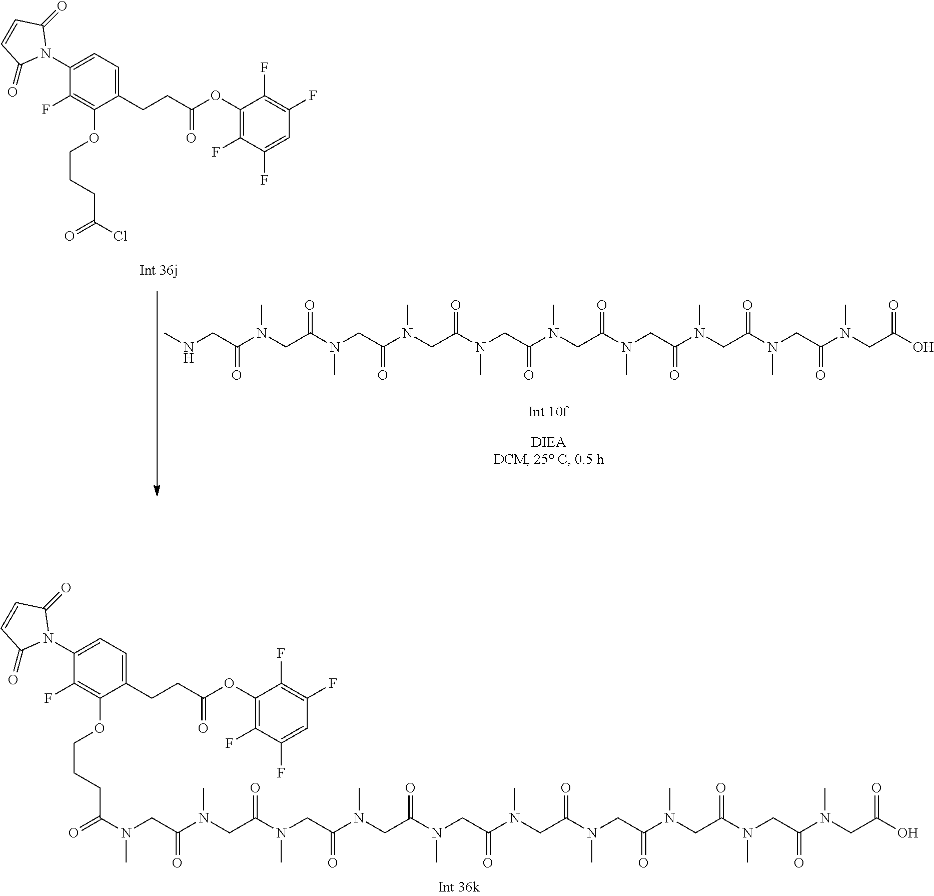 [SMILES: O=C(Cl)CCCOc1c(CCC(=O)Oc2c(F)c(F)cc(F)c2F)ccc(N2C(=O)C=CC2=O)c1F ; CNCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O ; CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCCOc1c(CCC(=O)Oc2c(F)c(F)cc(F)c2F)ccc(N2C(=O)C=CC2=O)c1F]

To a solution of 5,8,11,14,17,20,23,26,29-nonamethyl-4,7,10,13,16,19,22,25,28-nonaoxo-2,5,8,11,14,17,20,23,26,29-decaazahentriacontan-31-oic acid, Int 10f, (137 mg, 188 mol, 1.00 equiv) in dichloromethane (2.00 mL) and N,N-diisopropylethylamine (72.9 mg, 564 mol, 98.3 μL, 3.00 equiv). The mixture was stirred at 25° C. for 10 min. Then to the mixture was added 2,3,5,6-tetrafluorophenyl 3-(2-(4-chloro-4-oxobutoxy)-4-(2,5-dioxo-2,5-dihydro-1H-pyrrol-1-yl)-3-fluorophenyl)propanoate, Int. 36j, (100 mg, 188 μmol, 1.00 equiv) at 0° C. The mixture was stirred at 25° C. for 20 min. The reaction mixture was concentrated under reduced pressure to give a crude product, which was purified by prep-HPLC (column: UniSil 3-100 C18 Ultra (150*25 mm*3 um); mobile phase: [water (formic acid)-acetonitrile]; gradient: 20%-50% B over 40 min). The desired fraction collected and lyophilized to afford 34-(3-(2,5-dioxo-2,5-dihydro-1H-pyrrol-1-yl)-2-fluoro-6-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, Int. 36k, (30.0 mg, 24.3 gmol, 12% yield, 99% purity) as a white solid. ¹H NMR (400 MHz, DMSO-d₆) δ=13.48-12.07 (m, 1H), 8.00-7.87 (m, 1H), 7.26 (s, 2H), 7.25-7.20 (m, 1H), 7.13-7.06 (m, 1H), 4.34-4.19 (m, 8H), 4.15-3.98 (m, 1H), 3.93 (br d, J=4.4 Hz, 2H), 3.16 (br d, J=6.0 Hz, 2H), 3.11-3.06 (m, 2H), 2.95-2.71 (m, 31H), 2.35-2.29 (m, 2H), 1.99-1.89 (m, 2H). MS (ESI) m/z 1246.4 [M+Na]⁺

### Step L. Preparation of Int 36l

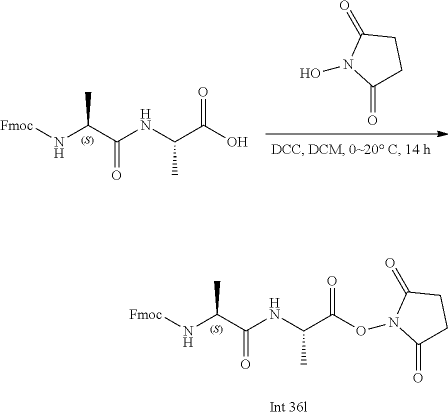

To a solution of (2S)-2-[[(2S)-2-(9H-fluoren-9-ylmethoxycarbonylamino)propanoyl]amino]propanoic acid (10 g, 26.2 mmol, 1 equiv) in DCM (100 mL) was added 1-hydroxypyrrolidine-2,5-dione (9.03 g, 78.5 mmol, 3 equiv) and DCC (16.2 g, 78.5 mmol, 15.9 mL, 3 equiv) at 0° C. The mixture was allowed to warm to 20° C. and stirred for 14 hours. The reaction solution was filtered and the filtrate was concentrated under reduced pressure to give a residue. Compound (2,5-dioxopyrrolidin-1-yl) (2S)-2-[[(2S)-2-(9H-fluoren-9-ylmethoxycarbonylamino)propanoyl]amino]propanoate, Int. 361, (22.5 g, crude) was obtained as a white solid.

### Step M. Preparation of Int 50m

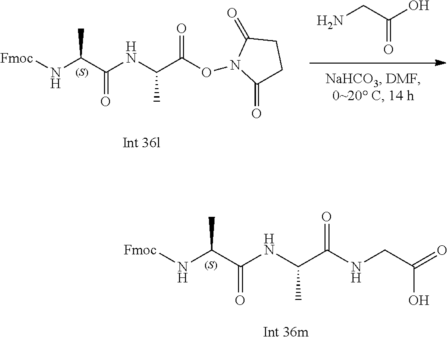

To a solution of (2,5-dioxopyrrolidin-1-yl) (2S)-2-[[(2S)-2-(9H-fluoren-9-ylmethoxycarbonylamino)propanoyl]amino]propanoate, Int. 361, (45 g, 93.9 mmol, 1 equiv) and 2-aminoacetic acid (7.05 g, 93.9 mmol, 1 equiv) in DMF (250 mL) was added the solution of NaHCO₃ (23.7 g, 282 mmol, 11.0 mL, 3 equiv) in H₂O (250 mL) at 0° C. The mixture was stirred at 20° C. for 12 hours. The reaction mixture was quenched with HCl (1 M) until pH=~2, and diluted with H₂O (500 mL). The aqueous was extracted with EtOAc (400 ml*3). The organic s were combined, and washed with brine (800 mL), dried over Na₂SO₄, filtered and concentrated under reduced pressure to give a residue. The residue was purified by flash silica gel chromatography (ISCO®; 220 g SepaFlash® Silica Flash Column, Eluent of 0~100% Ethyl acetate/Petroleum ether then 0-25% MeOH/Ethyl acetate gradient @100 mL/min). Compound 2-[[(2S)-2-[[(2S)-2-(9H-fluoren-9-ylmethoxycarbonylamino)propanoyl]amino]propanoyl]amino]acetic acid, Int. 36m, (12.6 g, 28.7 mmol, 30.6% yield) was obtained as a white solid. ¹H NMR (400 MHz, DMSO-d₆) δ 8.19-7.98 (m, 2H), 7.89 (d, J=7.6 Hz, 2H), 7.72 (t, J=7.2 Hz, 2H), 7.62-7.48 (m, 1H), 7.44-7.38 (m, 2H), 7.36-7.29 (m, 2H), 4.36-4.18 (m, 4H), 4.12-4.02 (m, 1H), 3.78-3.69 (m, 2H), 1.25-1.17 (m, 6H).

### Step N. Preparation of Int 36n

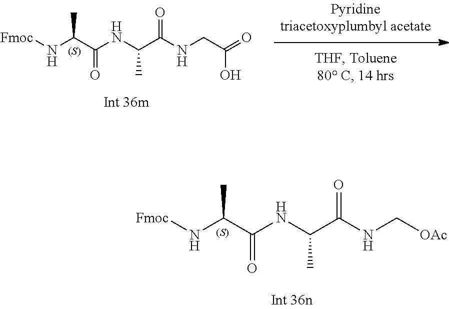

To a solution of 2-[[(2S)-2-[[(2S)-2-(9H-fluoren-9-ylmethoxycarbonylamino)propanoyl]amino]propanoyl]amino]acetic acid, Int. 36m, (7.60 g, 17.3 mmol, 1.0 equiv) in THF (90 mL) and toluene (30 mL) was added pyridine (1.64 g, 20.8 mmol, 1.68 mL, 1.2 equiv) and triacetoxyplumbyl acetate (9.20 g, 20.8 mmol, 1.2 equiv) at 25° C. under N₂. The reaction mixture was heated to 80° C. and stirred for 14 hours. The reaction mixture was filtered and the filtrate was concentrated under reduced pressure to give a residue. The residue was purified by column chromatography (SiO₂, Petroleum ether/Ethyl acetate=10/1 to 0/1). Compound [[(2S)-2-[[(2S)-2-(9H-fluoren-9-ylmethoxycarbonylamino)propanoyl]amino]propanoyl-]amino]methyl acetate, Int. 36m, (4.0 g, 8.82 mmol, 51.0% yield) was obtained as a white solid. ¹H NMR: (400 MHz, DMSO-d₆) δ 8.88-8.79 (m, 1H), 7.89 (d, J=7.6 Hz, 2H), 7.72 (t, J=7.2 Hz, 2H), 7.45-7.38 (m, 2H), 7.36-7.29 (m, 2H), 5.19-4.99 (m, 2H), 4.35-4.17 (m, 4H), 4.12-4.04 (m, 1H), 2.01-1.93 (m, 3H), 1.25-1.18 (m, 6H)

### Step O. Preparation of Int 36o

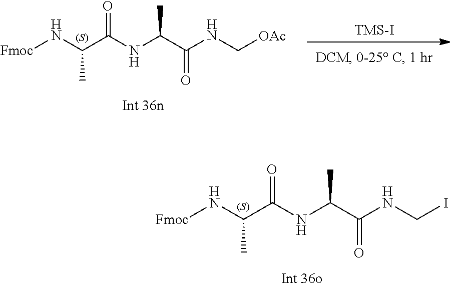

To a solution of [[(2S)-2-[[(2S)-2-(9H-fluoren-9-ylmethoxycarbonylamino)propanoyl]amino]propanoyl]amino]methyl acetate, Int. 36n, (0.6 g, 1.32 mmol, 1 equiv) in DCM (6 mL) was added TMSI (794 mg, 3.97 mmol, 540 μL, 3 equiv) at 0° C. The mixture was warmed to 25° C. and stirred for 1 hour. LC-MS showed Int 36n was consumed and ~80% of desired compound was detected (quenched with MeOH). The reaction mixture was concentrated under reduced pressure to give a residue. Compound 9H-fluoren-9-ylmethyl N-[(1S)-2-[[(1S)-2-(iodomethylamino)-1-methyl-2-oxo-ethyl]amino]-1-methyl-2-oxo-ethyl]carbamate, Int. 36o, (0.75 g, crude) was obtained as a red solid.

### Step P. Preparation of Int 36p

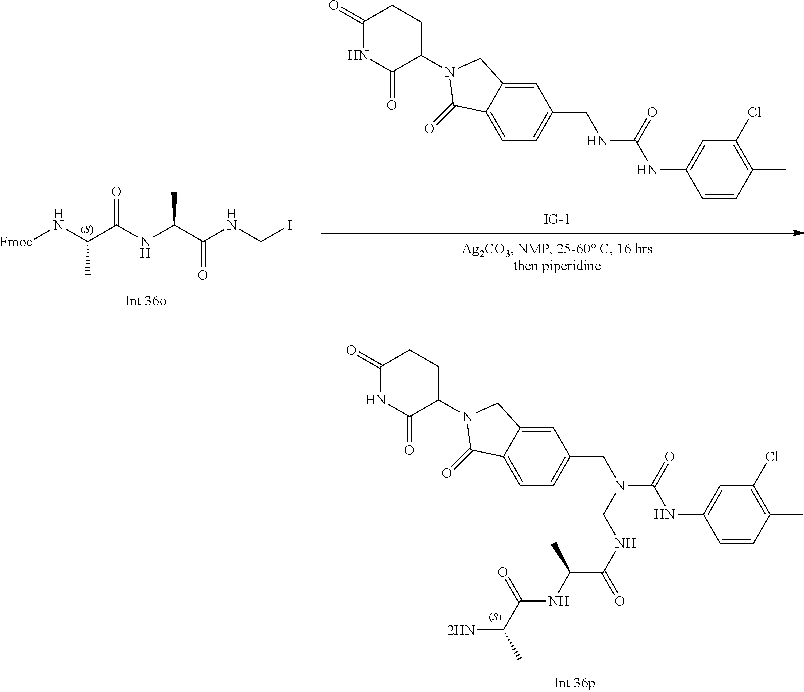

To a solution of 1-(3-chloro-4-methyl-phenyl)-3-[[2-(2,6-dioxo-3-piperidyl)-1-oxo-isoindolin-5-yl]methyl]urea, Compound IG-1, (0.15 g, 340 gmol, 1 equiv) in NMP (5 mL) was added Ag₂CO₃ (187 mg, 680 gmol, 30.9 μL, 2 equiv) and 9H-fluoren-9-ylmethyl N-[(1S)-2-[[(1S)-2-(iodomethylamino)-1-methyl-2-oxo-ethyl]amino]-1-methyl-2-oxo-ethyl]carbamate, Int. 50o, (709 mg, 1.36 mmol, 4 equiv) at 25° C. The mixture was heated to 60° C. and stirred for 16 hours under N₂ atmosphere. After that, the reaction mixture was cooled to 25° C., piperidine (127 mg, 1.50 mmol, 148 μL, 5 equiv) was added to the above mixture, and stirred at 25° C. for another 1 hour. The resulting mixture was filtered and purified by prep-HPLC (column: Phenomenex Luna C18 75×30 mm×3 um; mobile phase: [H₂O (0.1% TFA)-ACN]; gradient: 10%-40% B over 8.0 min). Compound (2S)-2-amino-N-((2S)-1-(((3-(3-chloro-4-methylphenyl)-1-((2-(2,6-dioxopiperidin-3-yl)-1-oxoisoindolin-5-yl)methyl)ureido)methyl)amino)-1-oxopropan-2-yl)propanamide, Int. 36p, (30 mg, 49.0 gmol, 16.4% yield) was obtained as a white solid. ¹H NMR (400 MHz, DMSO-d₆) δ 10.99 (s, 1H), 9.59 (d, J=16.4 Hz, 1H), 9.19-9.17 (m, 1H), 8.74-8.69 (m, 1H), 8.19-8.05 (m, 2H), 7.75-7.71 (m, 1H), 7.62 (d, J=2.0 Hz, 1H), 7.52-7.50 (m, 1H), 7.45-7.43 (m, 1H), 7.28-7.24 (m, 2H), 5.14-5.09 (m, 1H), 4.70-4.65 (m, 4H), 4.48-4.30 (m, 3H), 3.87-3.86 (m, 1H), 2.92-2.79 (m, 1H), 2.68-2.58 (m, 1H), 2.41-2.34 (m, 1H), 2.26 (s, 3H), 2.07-2.00 (m, 1H), 1.36-1.24 (m, 6H).

### Step Q. Preparation of Compound IG-L-36

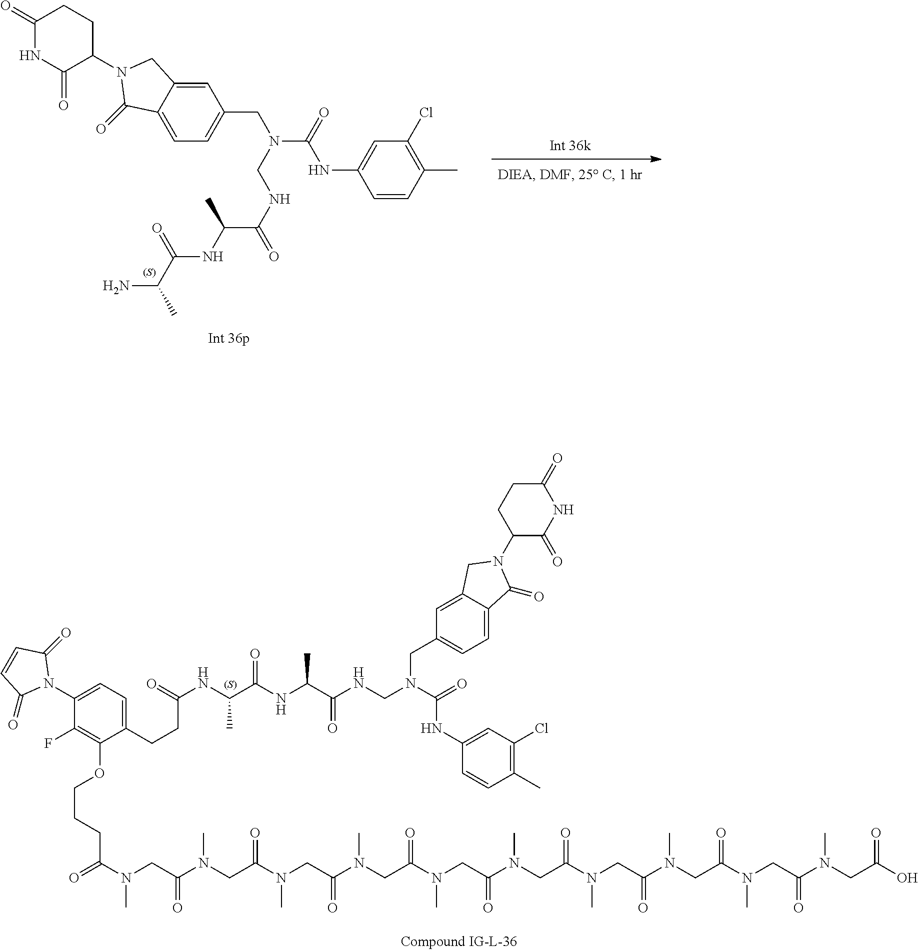

To a solution of (2S)-2-amino-N-((2S)-1-(((3-(3-chloro-4-methylphenyl)-1-((2-(2,6-dioxopiperidin-3-yl)-1-oxoisoindolin-5-yl)methyl)ureido)methyl)amino)-1-oxopropan-2-yl)propanamide, Int. 36p, (25 mg, 34.4 gmol, 1 equiv, TFA) in DMF (1 mL) was added DIEA (13.3 mg, 103 gmol, 18.00 μL, 3 equiv) and 2-[[2-[[2-[[2-[[2-[[2-[[2-[[2-[[2-[[2-[4-[3-(2,5-dioxopyrrol-1-yl)-2-fluoro-6-[3-oxo-3-(2,3,5,6-tetrafluorophenoxy)propyl]phenoxy]butanoyl-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetic acid, Int. 36k, (42.2 mg, 34.4 gmol, 1 equiv) at 0° C. The mixture was warmed to 25° C. and stirred for 1 hour. The reaction solution was quenched with TFA until pH=6. The resulting mixture was filtered and purified by prep-HPLC (column: Phenomenex Luna C18 75×30 mm×3 um; mobile phase: [H₂O (0.1% TFA)-ACN]; gradient: 20%-50% B over 8.0 min). Compound 34-(6-((6S,9S)-1-((3-chloro-4-methylphenyl)amino)-2-((2-(2,6-dioxopiperidin-3-yl)-1-oxoisoindolin-5-yl)methyl)-6,9-dimethyl-1,5,8,11-tetraoxo-2,4,7,10-tetraazatridecan-13-yl)-3-(2,5-dioxo-2,5-dihydro-1H-pyrrol-1-yl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, Compound IG-L-36, (31 mg, 18.6 μmol, 53.9% yield) was obtained as a white solid. ¹H NMR (400 MHz, DMSO-d₆) δ 10.98 (d, J=4.0 Hz, 1H), 9.80-9.64 (m, 1H), 9.12-8.93 (m, 1H), 8.50-8.07 (m, 2H), 7.76-7.67 (m, 1H), 7.65-7.59 (m, 1H), 7.55-7.49 (m, 1H), 7.45 (d, J=8.0 Hz, 1H), 7.29-7.18 (m, 3H), 7.14-6.98 (m, 2H), 5.17-5.04 (m, 1H), 4.74-4.50 (m, 4H), 4.47-3.84 (m, 26H), 3.10-2.50 (m, 35H), 2.45-2.30 (m, 3H), 2.22 (ks, 3H), 2.05-1.81 (m, 3H), 1.34-1.10 (m, 6H). MS (ESI): mass calcd. For C₇₆H₉₈ClFN₁₈O₂₂ 1668.68, m/z found 1669.6 [M+H]⁺.

### Example L-37: Synthesis of 34-(6-((9S,12S)-1-((3-chloro-4-methylphenyl)amino)-2-((2-(2,6-dioxopiperidin-3-yl)-1-oxoisoindolin-5-yl)methyl)-9,12-dimethyl-1,5,8,11,14-pentaoxo-2,4,7,10,13-pentaazahexadecan-16-yl)-3-(2,5-dioxo-2,5-dihydro-1H-pyrrol-1-yl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, IG-L-37)

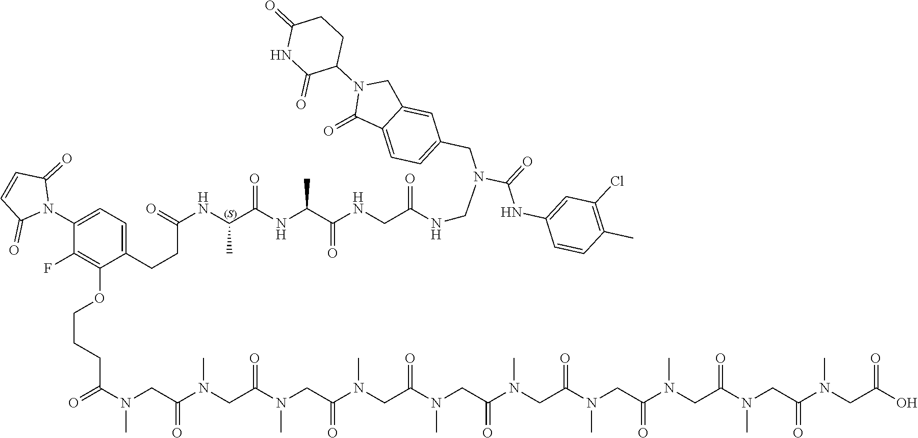

### Step A. Preparation of Int 37a

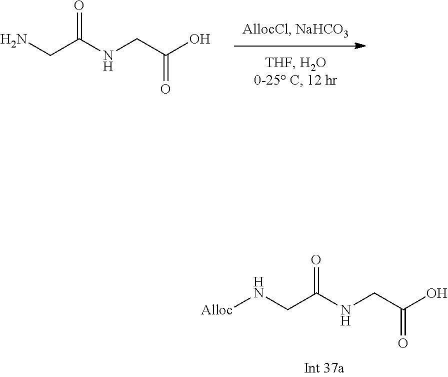

To a stirred reaction mixture of 2-[(2-aminoacetyl)amino]acetic acid (5.00 g, 37.9 mmol, 1.0 equiv) and NaHCO₃ (6.36 g, 75.7 mmol, 2.95 mL, 2.0 equiv) in H₂O (50 mL) was added allyl carbonochloridate (5.47 g, 45.4 mmol, 4.82 mL, 1.2 equiv) in THE (25 mL) dropwise at 0° C. The resulting mixture was warmed to 25° C. and stirred for 12 hours. The reaction mixture was concentrated to remove THF, the aqueous phase was acidified to pH=~5 with HCl (aq. 1 N). The precipitated solid was collected by filtration and washed with HCl (aq. 1 N) (50 mL×2). Compound 2-[[2-(allyloxycarbonylamino)acetyl]amino]acetic acid, Int. 37a, (3.00 g, 13.9 mmol, 36.7% yield) was obtained as a white solid. ¹H NMR (400 MHz, DMSO-d₆) δ 12.55 (s, 1H), 8.13 (t, J=5.6 Hz, 1H), 7.43 (t, J=6.0 Hz, 1H), 5.99-5.82 (m, 1H), 5.29 (dd, J=1.6, 17.2 Hz, 1H), 5.17 (dd, J=1.2, 10.4 Hz, 1H), 4.47 (td, J=1.6, 5.2 Hz, 2H), 3.75 (d, J=6.0 Hz, 2H), 3.63 (d, J=6.0 Hz, 2H)

### Step B. Preparation of Int 37b

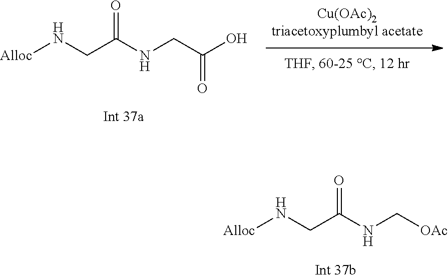

A mixture of 2-[[2-(allyloxycarbonylamino)acetyl]amino]acetic acid, Int. 37a, (2.80 g, 12.9 mmol, 1.0 equiv) and Cu(OAc)₂ (235 mg, 1.30 mmol, 0.1 equiv) in THF (30 mL) was stirred for 1 hour at 60° C. under nitrogen atmosphere. The mixture was allowed to cool down to 25° C. To the above mixture was added triacetoxyplumbyl acetate (6.89 g, 15.5 mmol, 1.2 equiv) at 25° C. The resulting mixture was stirred for an additional 12 hours at 25° C. The resulting mixture was filtered, the filter cake was washed with MeOH (50 mL×3). The filtrate was concentrated under reduced pressure. The residue was purified by silica gel column chromatography (SiO₂, Petroleum ether/Ethyl acetate=100/1 to 10/1). Compound [[2-(allyloxycarbonylamino)acetyl]amino]methyl acetate, Int. 37b, (2.40 g, 10.4 mmol, 80.5% yield) was obtained as a white solid. ¹H NMR (400 MHz, DMSO-d₆) δ 8.91 (t, J=6.8 Hz, 1H), 7.42 (t, J=6.0 Hz, 1H), 5.97-5.83 (m, 1H), 5.29 (dd, J=1.6, 17.2 Hz, 1H), 5.18 (dd, J=1.2, 10.4 Hz, 1H), 5.09 (d, J=7.2 Hz, 2H), 4.47 (td, J=1.2, 5.2 Hz, 2H), 3.63 (d, J=6.4 Hz, 2H), 1.99 (s, 3H)

### Step C. Preparation of Int 37c

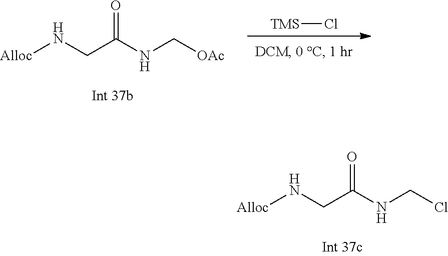

To a stirred mixture of [[2-(allyloxycarbonylamino)acetyl]amino]methyl acetate, Int. 37b, (0.9 g, 3.91 mmol, 1.0 equiv) in DCM (10 mL) was added TMSCl (1.70 g, 15.6 mmol, 1.98 mL, 4.0 equiv) dropwise at 0° C., and stirred for 1 hour, the reaction mixture was concentrated under reduced pressure. The crude product allyl N-[2-(chloromethylamino)-2-oxo-ethyl]carbamate, Int. 37c, (1 g crude) was used in the next step without further purification.

### Step D. Preparation of Int 37d

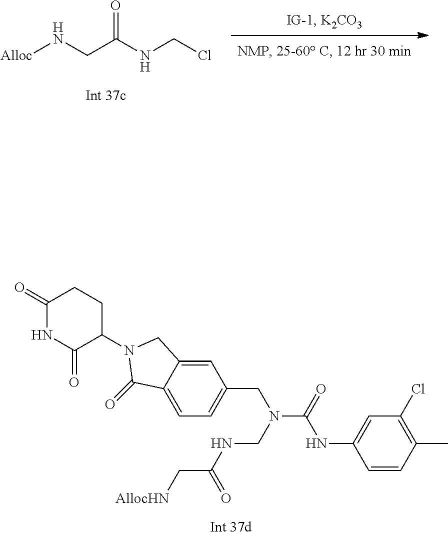

A mixture of 1-(3-chloro-4-methyl-phenyl)-3-[[2-(2,6-dioxo-3-piperidyl)-1-oxo-isoindolin-5-yl]methyl]urea, Compound IG-1, (0.15 g, 340 μmol, 1.0 equiv) and K₂CO₃ (94.0 mg, 680 μmol, 2.0 equiv) in NMP (5 mL) was stirred for 10 min at 25° C. To the above mixture was added allyl N-[2-(chloromethylamino)-2-oxo-ethyl]carbamate, Int. 37c, (422 mg, 2.04 mmol, 6.0 equiv) at 25° C. The resulting mixture was stirred for additional 12 hours at 60° C. The reaction mixture was poured into ice-water (w/w=1/1) (15 mL) and stirred for 2 min. The aqueous phase was extracted with ethyl acetate (20 mL×3). The combined organic phase was dried with anhydrous Na₂SO₄, filtered and concentrated in vacuum. The crude product allyl (2-(((3-(3-chloro-4-methylphenyl)-1-((2-(2,6-dioxopiperidin-3-yl)-1-oxoisoindolin-5-yl)methyl)ureido)methyl)amino)-2-oxoethyl)carbamate, Int. 37d, (200 mg, crude) was used into the next step without further purification.

### Step E. Preparation of Int 37e

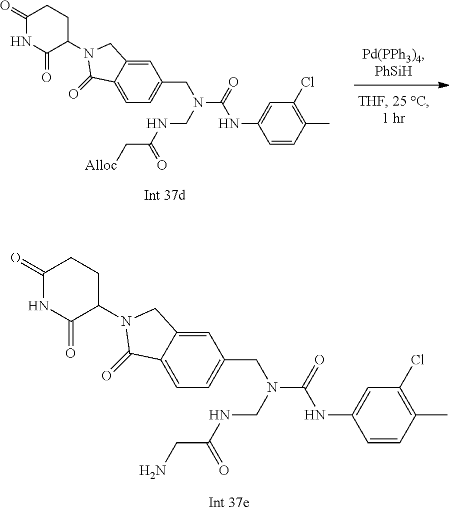

To a stirred mixture of allyl (2-(((3-(3-chloro-4-methylphenyl)-1-((2-(2,6-dioxopiperidin-3-yl)-1-oxoisoindolin-5-yl)methyl)ureido)methyl)amino)-2-oxoethyl)carbamate, Int. 37d, (250 mg, 409 μmol, 1.0 equiv) and Pd(PPh₃)₄ (47.3 mg, 40.9 μmol, 0.1 equiv) in THF (3 mL) was added phenylsilane (88.6 mg, 818 μmol, 101 μL, 2.0 equiv) dropwise at 25° C. under nitrogen atmosphere. The resulting mixture was stirred for 1 hour at 25° C. under nitrogen atmosphere. The residue was poured into ice-water (w/w=1/1) (4 mL) and stirred for 2 min. The aqueous phase was extracted with ethyl acetate (10 mL×3) and filtered. The aqueous phase was purified by prep-HPLC (column: Phenomenex Luna C18 75×30 mm×3 um; mobile phase: [H₂O (0.1% TFA)-ACN]; gradient: 5%-35% B over 8.0 min). Compound 2-amino-N-((3-(3-chloro-4-methylphenyl)-1-((2-(2,6-dioxopiperidin-3-yl)-1-oxoisoindolin-5-yl)methyl)ureido)methyl)acetamide, Int. 37e, (12.0 mg, 22.8 μmol, 5.57% yield) was obtained as a white solid. ¹H NMR (400 MHz, DMSO-d₆) δ 10.98 (s, 1H), 9.27 (s, 1H), 9.18 (s, 1H), 8.02 (s, 3H), 7.74 (d, J=8.0 Hz, 1H), 7.64 (d, J=2.0 Hz, 1H), 7.52 (s, 1H), 7.44 (d, J=8.0 Hz, 1H), 7.33 (dd, J=2.0, 8.4 Hz, 1H), 7.24 (d, J=8.4 Hz, 1H), 5.11 (dd, J=5.2, 13.2 Hz, 1H), 4.76 (d, J=6.4 Hz, 2H), 4.70 (s, 2H), 4.44 (s, 1H), 4.36-4.28 (m, 1H), 3.65 (s, 2H), 2.97-2.86 (m, 2H), 2.68-2.65 (m, 1H), 2.35-2.30 (m, 1H), 2.26 (s, 3H), 2.03-1.98 (m, 1H).

### Step F. Preparation of Int 37f

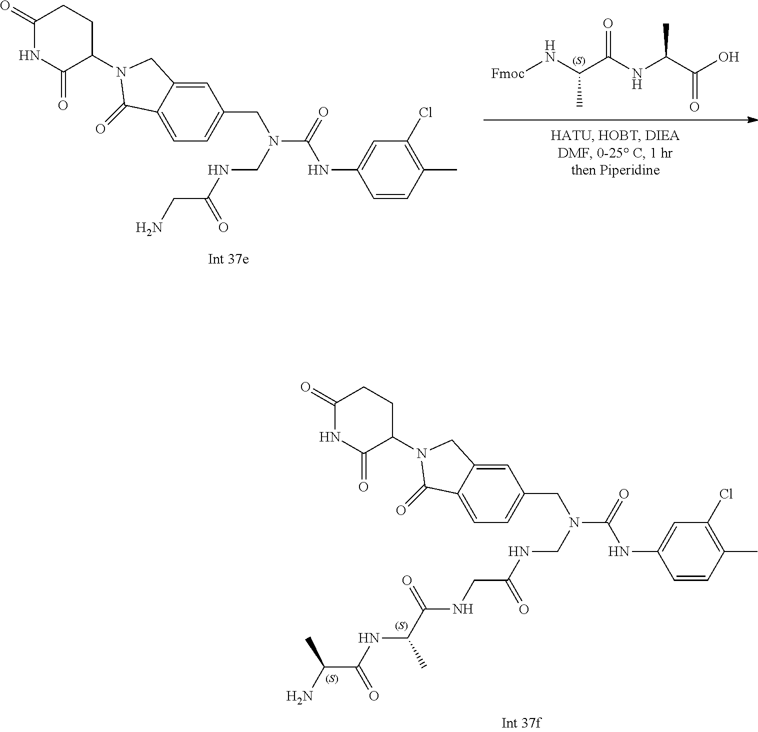

To a stirred mixture of (2S)-2-[[(2S)-2-(9H-fluoren-9-ylmethoxycarbonylamino)propanoyl]amino]propanoic acid (7.98 mg, 20.9 μmol, 1.0 equiv), HATU (9.52 mg, 25.1 μmol, 1.2 equiv) and HOBt (3.38 mg, 25.1 gmol, 1.2 equiv) in DMF (1 mL) was added 2-amino-N-((3-(3-chloro-4-methylphenyl)-1-((2-(2,6-dioxopiperidin-3-yl)-1-oxoisoindolin-5-yl)methyl) ureido) methyl)acetamide, Int. 37e, (11.0 mg, 20.9 μmol, 1.0 equiv), DIEA (8.09 mg, 62.6 μmol, 10.9 μL, 3.0 equiv) at 0° C. The resulting mixture was warm to 25° C. and stirred for 1 hour. After that, piperidine (7.64 mg, 89.8 μmol, 8.86 μL, 5.0 equiv) was added above mixture and stirred at 25° C. for 30 min. The mixture was quenched with TFA until pH=~6 at 0° C. The resulting mixture was filtered and purified by prep-HPLC (column: Phenomenex Luna C18 75×30 mm×3 um; mobile phase: [H₂O (0.1% TFA)-ACN]; gradient: 7%-37% B over 8.0 min). Compound (2S)-2-amino-N-((2S)-1-((2-(((3-(3-chloro-4-methylphenyl)-1-((2-(2,6-dioxopiperidin-3-yl)-1-oxoisoindolin-5-yl)methyl)ureido)methyl)amino)-2-oxoethyl)amino)-1-oxopropan-2-yl)propanamide, Int. 37f, (5.00 mg, 7.47 μmol, 41.6% yield) was obtained as a white solid.

### Step G. Preparation of Compound IG-L-37

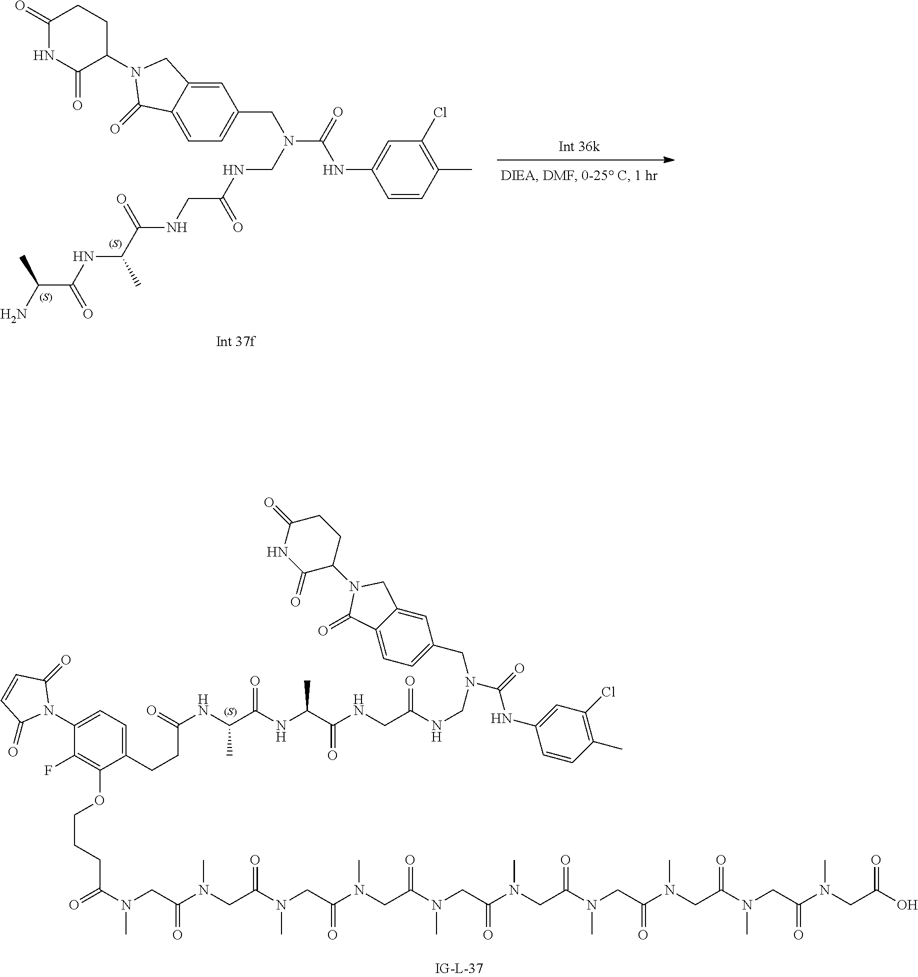

To a solution of (2S)-2-amino-N-((2S)-1-((2-(((3-(3-chloro-4-methylphenyl)-1-((2-(2,6-dioxopiperidin-3-yl)-1-oxoisoindolin-5-yl)methyl)ureido)methyl)amino)-2-oxoethyl)amino)-1-oxopropan-2-yl)propanamide, Int. 37f, (5.0 mg, 6.38 μmol, 1.0 equiv, TFA) in DMF (1 mL) was added DIEA (2.48 mg, 19.2 μmol, 3.34 μL, 3.0 equiv) and 2-[[2-[[2-[[2-[[2-[[2-[[2-[[2-[[2-[[2-[4-[3-(2,5-dioxopyrrol-1-yl)-2-fluoro-6-[3-oxo-3-(2,3,5,6-tetrafluorophenoxy)propyl]phenoxy]butanoyl-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetic acid, Int. 36k, (7.82 mg, 6.38 μmol, 1.0 equiv) at 0° C. The mixture was warm to 25° C. and stirred for 1 hour. The mixture was quenched with TFA until pH=~6 at 0° C. The resulting mixture was filtered and purified by prep-HPLC (column: Phenomenex Luna C18 75×30 mm×3 um; mobile phase: [H₂O (0.1% TFA)-ACN]; gradient: 15%-45% B over 8.0 min). Compound 34-(6-((9S,12S)-1-((3-chloro-4-methylphenyl)amino)-2-((2-(2,6-dioxopiperidin-3-yl)-1-oxoisoindolin-5-yl)methyl)-9,12-dimethyl-1,5,8,11,14-pentaoxo-2,4,7,10,13-pentaazahexadecan-16-yl)-3-(2,5-dioxo-2,5-dihydro-1H-pyrrol-1-yl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, Compound IG-L-37, (4.5 mg, 2.61 μmol, 40.8% yield) was obtained as a white solid. ¹H NMR (400 MHz, DMSO-d₆) δ=10.97 (s, 1H), 9.67-9.58 (m, 1H), 8.98-8.89 (m, 1H), 8.26-8.19 (m, 1H), 8.17-8.05 (m, 2H), 7.71 (d, J=8.0 Hz, 1H), 7.61 (d, J=2.0 Hz, 1H), 7.52 (s, 1H), 7.45 (d, J=8.4 Hz, 1H), 7.28-7.19 (m, 4H), 7.12-7.07 (m, 1H), 7.05-6.99 (m, 1H), 5.10 (dd, J=5.2, 13.2 Hz, 1H), 4.70-4.60 (m, 4H), 4.49-4.42 (m, 1H), 4.36-3.96 (m, 24H), 3.82-3.78 (m, 2H), 2.97-2.52 (m, 38H), 2.48-2.44 (m, 3H), 2.24 (s, 3H), 2.02-1.86 (m, 3H), 1.24 (d, J=7.2 Hz, 3H), 1.17 (d, J=7.2 Hz, 3H). MS (ESI): mass calcd. For C₇₈H₁₀₁ClFN₁₉O₂₃ 1725.70, m/z found 1726.7 [M+H]⁺.

### Example L-38: Synthesis of 34-(6-((9S)-9-benzyl-2-((2-(2,6-dioxopiperidin-3-yl)-4-hydroxy-1-oxoisoindolin-5-yl)methyl)-1-((3-isopropyl-4-methylphenyl)amino)-1,5,8,11,14,17-hexaoxo-2,4,7,10,13,16-hexaazanonadecan-19-yl)-3-(2,5-dioxo-2,5-dihydro-1H-pyrrol-1-yl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, IG-L-38

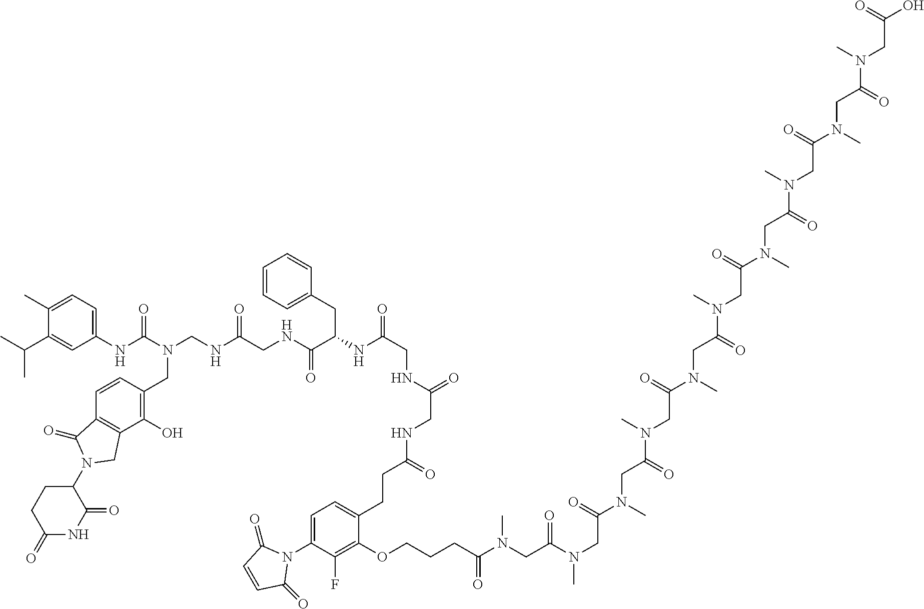 [SMILES: Cc1ccc(NC(=O)N(CNC(=O)CNC(=O)[C@H](Cc2ccccc2)NC(=O)CNC(=O)CNC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2ccc3c(c2O)CN(C2CCC(=O)NC2=O)C3=O)cc1C(C)C]

### Step A. Preparation of Int 38a

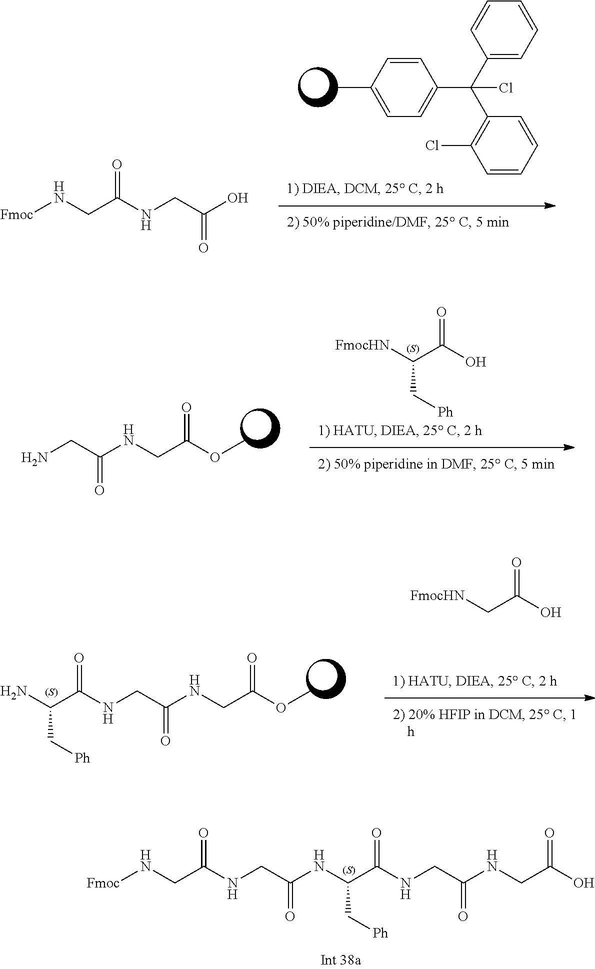

A solution of (((9H-fluoren-9-yl)methoxy)carbonyl)glycylglycine (10.0 g, 28.2 mmol, 1.00 eq) in dichloromethane (200 mL) was added to 2-chlorotrityl chloride resin, low crosslink, swellable polystyrene (60.0 g). Then diethylamine (9.12 g, 70.6 mmol, 12.3 mL, 2.50 eq) was added slowly and the mixture was agitated with nitrogen for 2 h at 25° C. The mixture was filtered and washed with dimethyl formamide (2×150 mL) and dichloromethane/methanol/diethylamine (150 mL, 80/15/5). Then the resin in piperidine (80.0 mL) and dimethyl formamide (80.0 mL) was agitated with nitrogen for 5 min at 25° C. The mixture was filtered and washed with dimethyl formamide (6×150 mL) to give product on the resin.

A solution of (((9H-fluoren-9-yl)methoxy)carbonyl)-L-phenylalanine (14.7 g, 37.9 mmol, 1.50 eq), diethylamine (6.54 g, 50.6 mmol, 8.82 mL, 2.00 eq) and O-(7-azabenzotriazol-1-yl)-N,N,N′,N′-tetramethyluronium hexafluorophosphate (17.3 g, 45.6 mmol, 1.80 eq) in dimethyl formamide (150 mL) was added to the resin. Then the mixture was agitated with nitrogen for 2 h at 25° C. The mixture was filtered and washed with dimethyl formamide (3×150 mL). The resin in piperidine (80.0 mL) and dimethyl formamide (80.0 mL) was agitated with nitrogen for 5 min at 25° C. The mixture was filtered and washed with dimethyl formamide (6×150 mL) to give product on the resin.

A solution of (((9H-fluoren-9-yl)methoxy)carbonyl)glycylglycine (13.4 g, 37.7 mmol, 1.50 eq), diethylamine (6.50 g, 50.3 mmol, 8.76 mL, 2.00 eq) and O-(7-Azabenzotriazol-1-yl)-N,N,N′,N′-tetramethyluronium hexafluorophosphate (17.2 g, 45.3 mmol, 1.80 eq) in dimethyl formamide (150 mL) was added to the resin. The mixture was agitated with nitrogen for 2 h at 25° C. The mixture was filtered and washed with dimethyl formamide (3×150 mL) and dichloromethane (3×150 mL). The resin in dichloromethane (150 mL) and hexafluoroisopropanol (30 mL) was agitated with nitrogen for 1 h at 25° C. The mixture was filtered. The filtrate was concentrated to give (((9H-fluoren-9-yl)methoxy)carbonyl)glycylglycyl-L-phenylalanylglycylglycine, Int-38a (15.0 g, crude) as an off-white solid. ¹H NMR (400 MHz, DMSO-d₆) δ=8.36 (br t, J=5.4 Hz, 1H), 8.17 (br d, J=8.0 Hz, 1H), 8.10-7.98 (m, 1H), 7.97-7.83 (m, 2H), 7.77-7.56 (m, 2H), 7.46-7.09 (m, 6H), 5.18 (td, J=6.8, 13.5 Hz, 1H), 4.63-4.44 (m, 1H), 4.35-4.11 (m, 2H), 3.87-3.51 (m, 6H), 3.20-2.89 (m, 2H), 2.84-2.72 (m, 2H). LCMS (ESI) m/z 616.3 [M+H]⁺

### Step B. Preparation of Int 38b

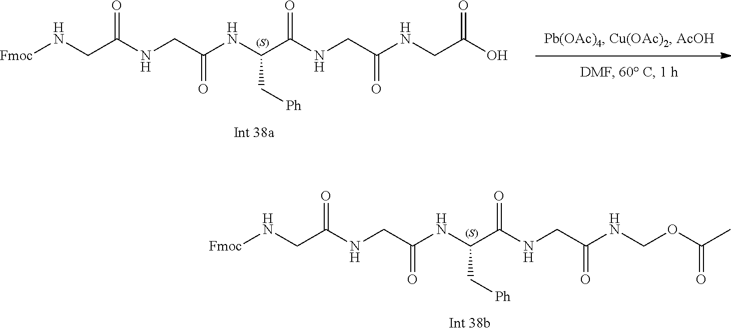

To a solution of (((9H-fluoren-9-yl)methoxy)carbonyl)glycylglycyl-L-phenylalanylglycylglycine (2.00 g, 3.25 mmol, 1.00 eq) and copper acetate (59.0 mg, 324 ∞mol, 0.10 eq) in dimethyl formamide (20.0 mL) were added acetic acid (390 mg, 6.50 mmol, 372 μL, 2.00 eq) and triacetoxyplumbyl acetate (2.16 g, 4.87 mmol, 1.5 eq). Then the mixture was stirred at 60° C. for 1 h. The reaction mixture was filtered. The filtrate was purified by reversed phase (C18, 330 g; condition: water/acetonitrile=100/0 to 0/100, 0.1% formic acid) and lyophilized to afford (S)-11-benzyl-1-(9H-fluoren-9-yl)-3,6,9,12,15-pentaoxo-2-oxa-4,7,10,13,16-pentaazaheptadecan-17-yl acetate, Int 38b (1.50 g, 2.38 mmol, 73% yield) as a white solid. ¹H NMR (400 MHz, DMSO-d₆) δ=8.86 (br t, J=6.8 Hz, 1H), 8.35 (br t, J=5.6 Hz, 1H), 8.17 (br d, J=8.4 Hz, 1H), 8.03 (br t, J=5.4 Hz, 1H), 7.89 (d, J=7.6 Hz, 2H), 7.71 (d, J=7.6 Hz, 2H), 7.61 (br t, J=6.0 Hz, 1H), 7.46-7.37 (m, 2H), 7.36-7.29 (m, 2H), 7.28-7.21 (m, 4H), 7.20-7.13 (m, 1H), 5.16-5.05 (m, 2H), 4.62-4.45 (m, 1H), 4.34-4.12 (m, 3H), 3.74 (br t, J=6.4 Hz, 3H), 3.70-3.51 (m, 3H), 3.05 (br dd, J=4.0, 13.8 Hz, 1H), 2.85-2.71 (m, 1H), 1.99 (s, 3H). LCMS (ESI) m/z 570.3 [M-OAc]⁺

### Step C. Preparation of Int 38c

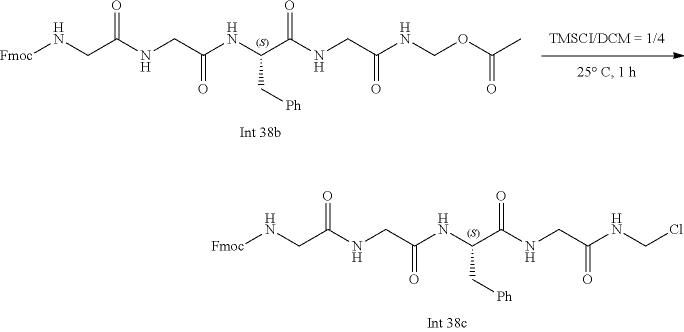

To a solution of (S)-11-benzyl-1-(9H-fluoren-9-yl)-3,6,9,12,15-pentaoxo-2-oxa-4,7,10,13,16-pentaazaheptadecan-17-yl acetate (200 mg, 318 μmol, 1.00 eq) in dichloromethane (4.00 mL) was added trimethylchlorosilane (1.00 mL). The mixture was stirred at 25° C. for 1 h. The reaction mixture was concentrated under reduced pressure to give (9H-fluoren-9-yl)methyl (S)-(7-benzyl-1-chloro-3,6,9,12-tetraoxo-2,5,8,11-tetraazatridecan-13-yl)carbamate, Int 38c (180 mg, 297 μmol, 94% yield) as white solid. MS (ESI) m/z 570.2 [M-Cl]⁺

### Step D. Preparation of Int 38d

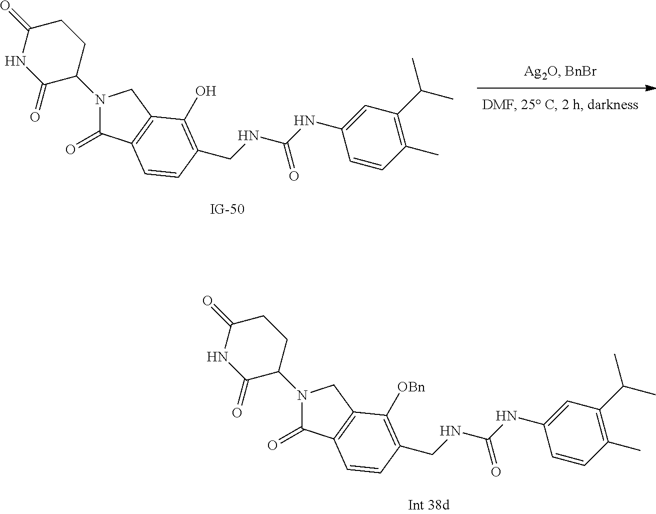 [SMILES: Cc1ccc(NC(=O)NCc2ccc3c(c2O)CN(C2CCC(=O)NC2=O)C3=O)cc1C(C)C ; Cc1ccc(NC(=O)NCc2ccc3c(c2OCc2ccccc2)CN(C2CCC(=O)NC2=O)C3=O)cc1C(C)C]

To a solution of 1-((2-(2,6-dioxopiperidin-3-yl)-4-hydroxy-1-oxoisoindolin-5-yl)methyl)-3-(3-isopropyl-4-methylphenyl)urea, IG-50, (250 mg, 538 μmol, 1.00 eq) in dimethyl formamide (5.00 mL) were added silver oxide (624 mg, 2.69 mmol, 5.00 eq) and benzyl bromide (276 mg, 1.61 mmol, 192 μL, 3.00 eq). The mixture was stirred at 25° C. for 2 h. The mixture was filtered. The filtrate was purified by reversed-phase HPLC (0.1% formic acid condition). The desired fraction was collected and lyophilized to give 1-((4-(benzyloxy)-2-(2,6-dioxopiperidin-3-yl)-1-oxoisoindolin-5-yl)methyl)-3-(3-isopropyl-4-methylphenyl)urea, Int 38d (165 mg, 297 mol, 27% yield) as a white solid. ¹H NMR (400 MHz, DMSO-d₆) δ=11.01 (br s, 1H), 8.64-8.57 (m, 1H), 7.54 (br d, J=7.2 Hz, 2H), 7.48-7.35 (m, 5H), 7.24 (d, J=2.0 Hz, 1H), 7.13 (dd, J=2.0, 8.4 Hz, 1H), 6.95 (d, J=8.4 Hz, 1H), 6.60 (br t, J=5.6 Hz, 1H), 5.22 (s, 2H), 5.17-5.06 (m, 1H), 4.67 (br d, J=17.2 Hz, 1H), 4.56-4.47 (m, 1H), 4.41 (br d, J=6.0 Hz, 2H), 3.02 (td, J=6.8, 13.2 Hz, 1H), 2.95-2.89 (m, 1H), 2.63 (br d, J=2.4 Hz, 1H), 2.47-2.42 (m, 1H), 2.19 (s, 3H), 2.05-1.98 (m, 1H), 1.13 (d, J=6.8 Hz, 6H). LC-MS (ESI) m/z 555.3 [M+H]⁺

### Step E. Preparation of Int 38e

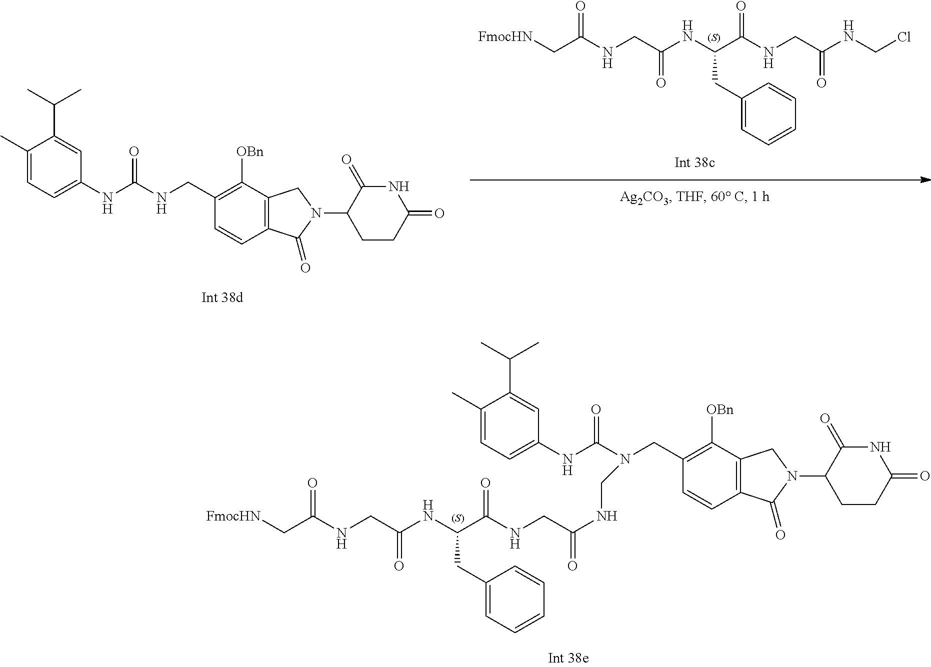

To a solution of 1-((4-(benzyloxy)-2-(2,6-dioxopiperidin-3-yl)-1-oxoisoindolin-5-yl)methyl)-3-(3-isopropyl-4-methylphenyl)urea, Int 38d (300 mg, 541 μmol, 1.00 eq) and (9H-fluoren-9-yl)methyl (S)-(7-benzyl-1-chloro-3,6,9,12-tetraoxo-2,5,8,11-tetraazatridecan-13-yl)carbamate (656 mg, 1.08 mmol, 2.00 eq) in tetrahydrofuran (10.0 mL) was added silver carbonate (447 mg, 1.62 mmol, 73.6 μL, 3.00 eq). The mixture was stirred at 60° C. for 1 h. The reaction mixture was concentrated under reduced pressure to give a residue. The residue was purified by reversed-phase HPLC (0.1% formic acid condition). The desired fraction was collected and lyophilized to give (9H-fluoren-9-yl) methyl ((9S)-9-benzyl-2-((4-(benzyloxy)-2-(2,6-dioxopiperidin-3-yl)-1-oxoisoindolin-5-yl)methyl)-1-((3-isopropyl-4-methylphenyl)amino)-1,5,8,11,14-pentaoxo-2,4,7,10,13-pentaazapentadecan-15-yl)carbamate, Int 38e (100 mg, 88.9 mol, 16% yield). ¹H NMR (400 MHz, DMSO-d₆) δ=11.00 (br s, 1H), 9.37-9.28 (m, 1H), 8.87 (br d, J=5.2 Hz, 1H), 8.43 (br d, J=6.4 Hz, 1H), 8.15 (br d, J=8.4 Hz, 1H), 8.02-7.97 (m, 1H), 7.88 (d, J=7.6 Hz, 2H), 7.69 (br d, J=7.2 Hz, 2H), 7.57-7.52 (m, 3H), 7.46 (d, J=7.6 Hz, 1H), 7.43-7.38 (m, 5H), 7.34-7.28 (m, 5H), 7.23-7.21 (m, 4H), 7.16 (br d, J=2.0 Hz, 1H), 6.96 (d, J=8.0 Hz, 1H), 5.23 (s, 2H), 5.13 (dd, J=5.2, 12.8 Hz, 1H), 4.74 (br d, J=6.4 Hz, 2H), 4.71 (br d, J=3.6 Hz, 2H), 4.55-4.49 (m, 2H), 4.29-4.26 (m, 2H), 4.22 (br d, J=6.4 Hz, 1H), 3.77 (br t, J=5.6 Hz, 2H), 3.72 (br d, J=5.2 Hz, 1H), 3.60 (br d, J=5.6 Hz, 2H), 3.09-2.89 (m, 4H), 2.76 (br s, 2H), 2.59 (br s, 1H), 2.45-2.41 (m, 1H), 2.19 (s, 3H), 2.02-1.96 (m, 1H), 1.09 (d, J=6.8 Hz, 6H). LC-MS (ESI) m/z 1124.5 [M+H]⁺

### Step F. Preparation of Int 38f

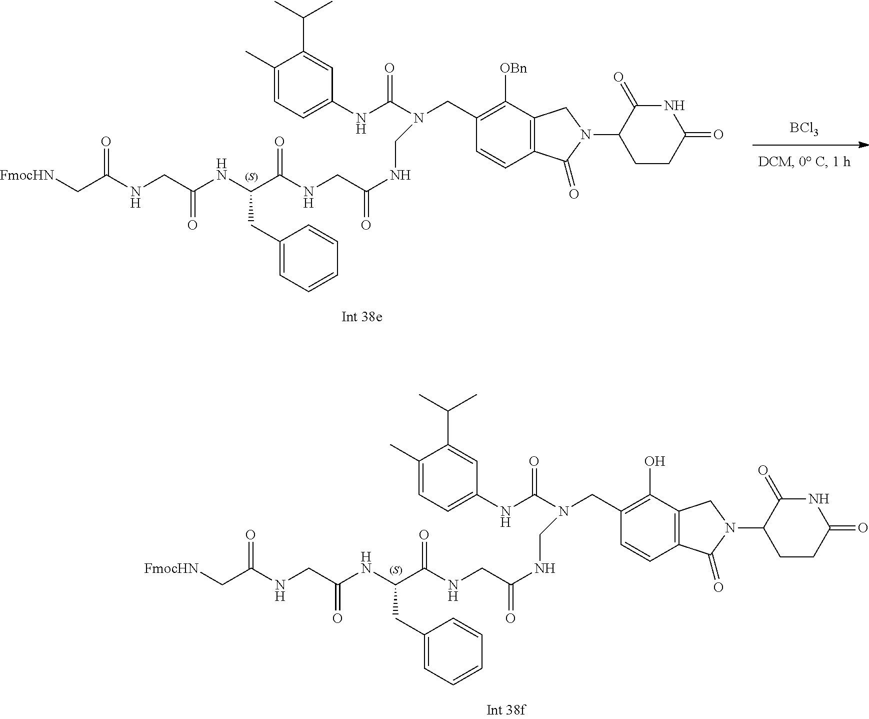

To a solution of (9H-fluoren-9-yl)methyl ((9S)-9-benzyl-2-((4-(benzyloxy)-2-(2,6-dioxopiperidin-3-yl)-1-oxoisoindolin-5-yl)methyl)-1-((3-isopropyl-4-methylphenyl)amino)-1,5,8,11,14-pentaoxo-2,4,7,10,13-pentaazapentadecan-15-yl)carbamate Int 38e (80.0 mg, 71.2 mol, 1.00 eq) in dichloromethane (5.00 mL) was added boron trichloride (1 M, 712 μL, 10.0 eq). The mixture was stirred at 0° C. for 1 h. The reaction mixture was diluted water (20.0 mL). The mixture was extracted with ethyl acetate (3×20 mL). The combined organic extracts were washed with brine (20.0 mL), dried over sodium sulfate and filtered. The filtrate was purified by reversed-phase HPLC (0.1% formic acid condition). The desired fraction was collected and lyophilized to give (9H-fluoren-9-yl)methyl ((9S)-9-benzyl-2-((2-(2,6-dioxopiperidin-3-yl)-4-hydroxy-1-oxoisoindolin-5-yl)methyl)-1-((3-isopropyl-4-methylphenyl)amino)-1,5,8,11,14-pentaoxo-2,4,7,10,13-pentaazapentadecan-15-yl)carbamate, Int 38f (30.0 mg, 29.0 μmol, 41% yield) as a white solid. LC-MS (ESI) m/z 1034.8 [M+H]⁺

### Step G. Preparation of Int 38g

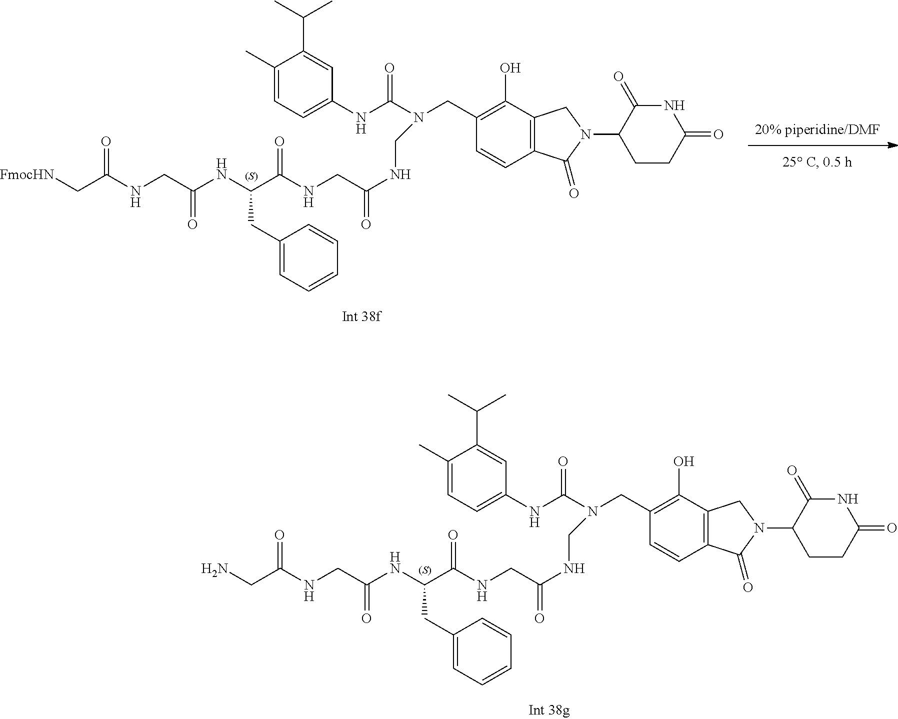

To a solution of (9H-fluoren-9-yl)methyl ((9S)-9-benzyl-2-((2-(2,6-dioxopiperidin-3-yl)-4-hydroxy-1-oxoisoindolin-5-yl)methyl)-1-((3-isopropyl-4-methylphenyl)amino)-1,5,8,11,14-pentaoxo-2,4,7,10,13-pentaazapentadecan-15-yl)carbamate Int 38f (50.0 mg, 48.4 μmol, 1.00 eq) in piperidine (0.500 mL) and dimethyl formamide (2.50 mL). The mixture was stirred at 25° C. for 0.5 h. The mixture was filtered. The filtrate was purified by prep-HPLC (column: Phenomenex luna C18 150*25 mm*10 um; mobile phase: [water (formic acid)-acetonitrile]; gradient: 24%-54% B over 10 min). The desired fraction was collected and lyophilized to give (2S)-2-(2-(2-aminoacetamido) acetamido)-N-(2-(((1-((2-(2,6-dioxopiperidin-3-yl)-4-hydroxy-1-oxoisoindolin-5-yl)methyl)-3-(3-isopropyl-4-methylphenyl)ureido)methyl)amino)-2-oxoethyl)-3-phenylpropanamide, Int 38g (20.0 mg, 24.6 μmol, 51% yield). LC-MS (ESI) m/z 812.6 [M+H]⁺

### Step H. Preparation of Compound IG-L-38

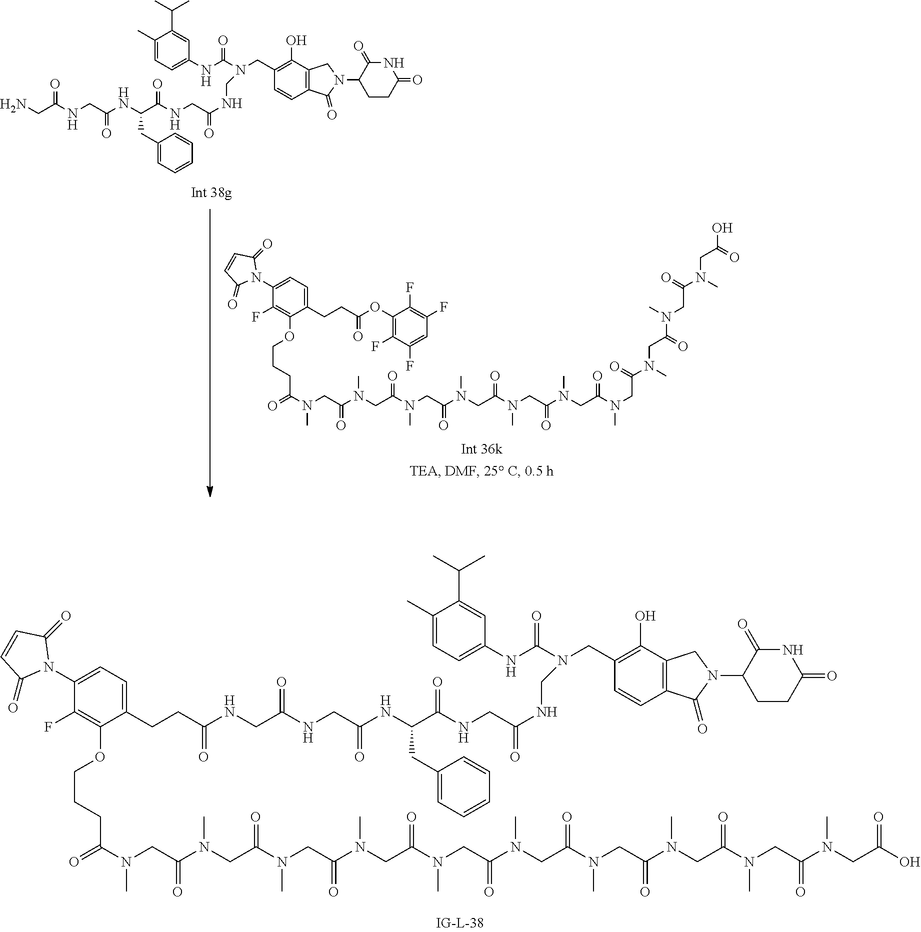

To a solution of (2S)-2-(2-(2-aminoacetamido)acetamido)-N-(2-(((1-((2-(2,6-dioxopiperidin-3-yl)-4-hydroxy-1-oxoisoindolin-5-yl)methyl)-3-(3-isopropyl-4-methylphenyl)ureido)methyl)amino)-2-oxoethyl)-3-phenylpropanamide, Int 38g (20.0 mg, 24.6 mol, 1.00 eq) and 34-(3-(2,5-dioxo-2,5-dihydro-1H-pyrrol-1-yl)-2-fluoro-6-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, Int 36k (30.2 mg, 24.6 μmol, 1.00 eq) in dimethyl formamide (2.00 mL) was added triethylamine (7.48 mg, 73.9 μmol, 10.3 μL, 3.00 eq). The mixture was stirred at 25° C. for 0.5 h. The mixture was filtered. The filtrate was purified by prep-HPLC (column: Phenomenex luna C18 150*25 mm*10 um; mobile phase: [water (formic acid)-acetonitrile]; gradient: 25%-55% B over 20 min). The desired fraction was collected and lyophilized to give 34-(6-((9S)-9-benzyl-2-((2-(2,6-dioxopiperidin-3-yl)-4-hydroxy-1-oxoisoindolin-5-yl)methyl)-1-((3-isopropyl-4-methylphenyl)amino)-1,5,8,11,14,17-hexaoxo-2,4,7,10,13,16-hexaazanonadecan-19-yl)-3-(2,5-dioxo-2,5-dihydro-TH-pyrrol-1-yl)-2-fluorophenoxy)-3,6,9,12,15,18,21,24,27,30-decamethyl-4,7,10,13,16, 19,22,25,28, 31-decaoxo-3,6,9,12,15,18,21,24,27,30-decaazatetratriacontanoic acid, Compound IG-L-38 (16.49 mg, 8.73 μmol, 35% yield, 99% purity) as a white solid. ¹H NMR (400 MHz, DMSO-d₆) δ=12.74 (s, 1H), 10.97 (s, 1H), 10.68 (br s, TH), 9.75-9.62 (m, 1H), 9.14-9.02 (m, TH), 8.55-8.44 (m, 1H), 8.25-8.03 (m, 3H), 7.44 (br d, J=7.6 Hz, 1H), 7.26-7.22 (m, 8H), 7.19-7.14 (m, 2H), 7.13-7.09 (m, 1H), 7.04-6.98 (m, 2H), 5.09 (dd, J=4.8, 13.2 Hz, 1H), 4.64 (br d, J=4.8 Hz, 2H), 4.51 (br s, 3H), 4.36-4.17 (m, 12H), 4.11-3.91 (m, 13H), 3.85-3.62 (m, 6H), 3.03 (br d, J=6.8 Hz, 1H), 2.96-2.71 (m, 37H), 2.61 (br d, J=2.8 Hz, 2H), 2.45 (br d, J=0.8 Hz, 1H), 2.20 (s, 3H), 2.03-1.87 (m, 3H), 1.10 (d, J=6.8 Hz, 6H). LC-MS (ESI) m/z 1870.7 [M+2H]⁺

The compounds in Table 17 were prepared in a manner similar to that described for Compound IG-L-38 using the appropriate compound as starting material.

TABLE 17

| IG-L No. | Structure | ¹H NMR/MS |
|---|---|---|
| IG-L-39 | 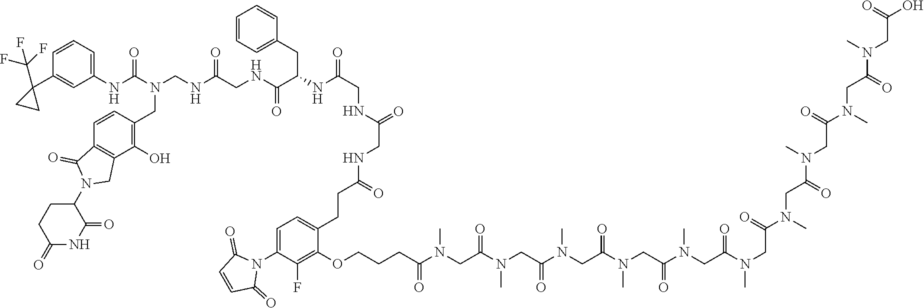 [SMILES: CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCCOc1c(CCC(=O)NCC(=O)NCC(=O)N[C@@H](Cc2ccccc2)C(=O)NCC(=O)NCN(Cc2ccc3c(c2O)CN(C2CCC(=O)NC2=O)C3=O)C(=O)Nc2cccc(C3(C(F)(F)F)CC3)c2)ccc(N2C(=O)C=CC2=O)c1F] | 1H NMR (400 MHz, DMSO-d6) δ = 12.77 (s, 1H), 10.98 (s, 1H), 10.50 (br s, 1H), 9.88 (br s, 1H), 9.08 (br s, 1H), 8.49 (br s, 1H), 8.34-8.13 (m, 2H), 8.07 (br d, J = 3.2 Hz, 1H), 7.58 (s, 1H), 7.43 (br d, J = 7.6 Hz, 2H), 7.31-7.22 (m, 8H), 7.18 (br dd, J = 3.6, 8.4 Hz, 1H), 7.14-7.00 (m, 3H), 5.10 (dd, J = 5.2, 13.2 Hz, 1H), 4.69 (br d, J = 6.0 Hz, 2H), 4.58-4.49 (m, 3H), 4.41-4.17 (m, 12H), 4.14-3.89 (m, 13H), 3.88-3.64 (m, 6H), 3.13-3.07 (m, 1H), 3.01-2.70 (m, 36H), 2.64-2.55 (m, 2H), 2.45-2.37 (m, 1H), 2.04-1.88 (m, 3H), 1.30-1.25 (m, 2H), 1.05 (br s, 2H). MS (ESI) m/z 1922.6 [M + 2H]⁺ |
| IG-L-43 | 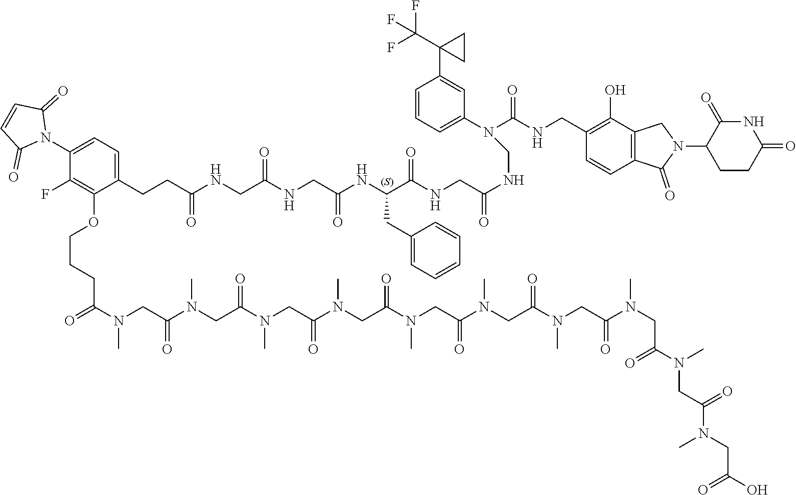 | ¹H NMR (400 MHz, DMSO-d₆) δ = 11.00 (s, 1H), 10.50-10.28 (m, 1H), 8.57-8.43 (m, 1H), 8.37-8.00 (m, 4H), 7.45-7.41 (m, 2H), 7.36 (s, 1H), 7.32-7.23 (m, 8H), 7.22-7.16 (m, 2H), 7.16-7.11 (m, 1H), 7.05 (t, J = 7.2 Hz, 1H), 7.00-6.91 (m, 1H), 5.10 (dd, J = 4.4, 13.2 Hz, 1H), 4.94 (br d, J = 5.2 Hz, 2H), 4.56-4.48 (m, 1H), 4.39-4.18 (m, 14H), 4.13-3.92 (m, 12H), 3.77-3.56 (m, 6H), 3.03-2.71 (m, 38H), 2.63-2.57 (m, 1H), 2.38-2.22 (m, 2H), 2.03-1.91 (m, 3H), 1.36-1.32 (m, 2H), 1.13 (br s, 2H). MS (ESI) m/z 961.8 [M/2 + H]+ |
| IG-L-76 | 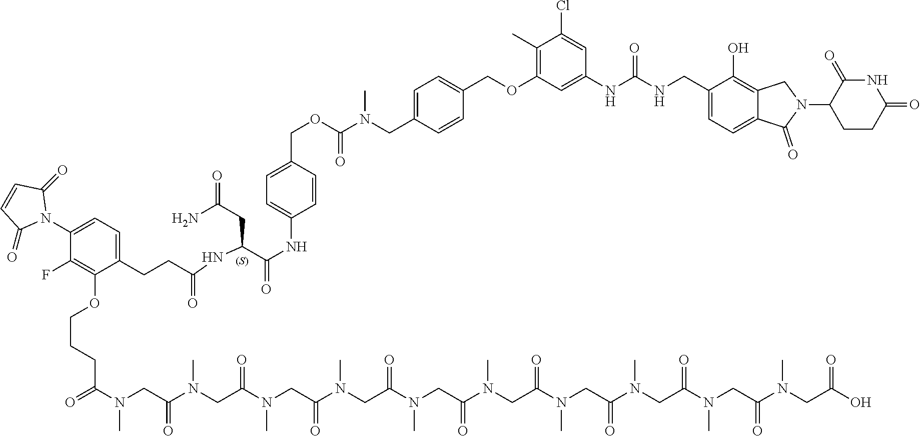 | ¹H NMR (400 MHz, DMSO-d₆) δ = 12.73 (s, 1H), 10.98 (s, 1H), 10.07-9.98 (m, 1H), 8.99-8.83 (m, 1H), 8.31-8.19 (m, 1H), 7.58 (br s, 2H), 7.46-7.40 (m, 2H), 7.37-7.29 (m, 3H), 7.29-7.17 (m, 7H), 7.15-7.09 (m, 1H), 7.04 (br s, 1H), 6.99 (br t, J = 7.2 Hz, 1H), 6.88 (br s, 1H), 5.13-5.00 (m, 5H), 4.71 (br d, J = 7.2 Hz, 1H), 4.44 (br s, 2H), 4.37- 4.17 (m, 13H), 4.11-3.91 (m, 12H), 3.04- 2.99 (m, 1H), 2.98-2.68 (m, 38H), 2.60 (br d, J = 16.4 Hz, 3H), 2.44-2.35 (m, 2H), 2.31-2.18 (m, 1H), 2.14 (s, 3H), 2.04-1.88 (m, 3H). LC-MS (ESI) m/z 964.2 [M/2 + 2H]⁺ |
| IG-L-77 | 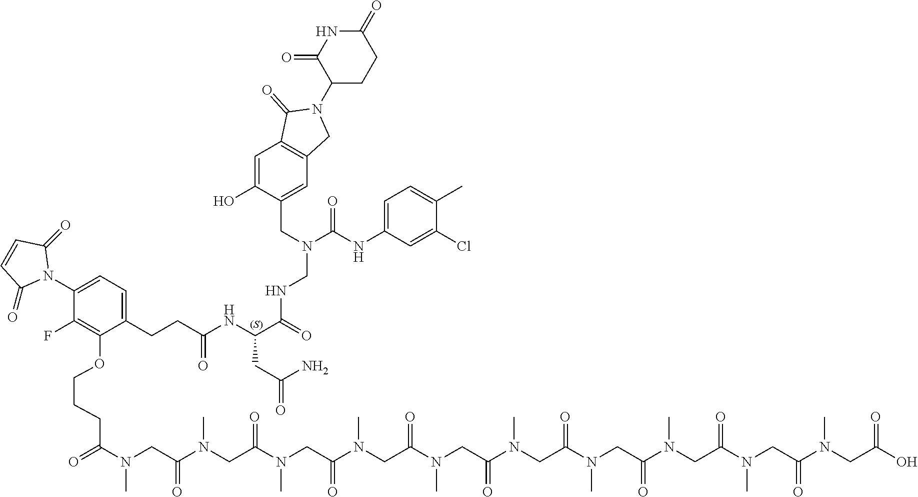 | ¹H NMR (400 MHz, DMSO-d₆) δ = 10.95 (s, 1H), 10.44-10.09 (m, 1H), 9.85-9.55 (m, 1H), 9.16-8.92 (m, 1H), 8.37-8.21 (m, 1H), 7.60 (s, 1H), 7.34-7.19 (m, 6H), 7.13-7.06 (m, 2H), 7.04-6.97 (m, 1H), 6.88 (br s, 1H), 5.05 (dd, J = 4.4, 12.8 Hz, 1H), 4.75-4.59 (m, 3H), 4.53 (br s, 2H), 4.36-4.16 (m, 12H), 4.11-3.89 (m, 14H), 2.99-2.69 (m, 38H), 2.24 (s, 3H), 2.00-1.87 (m, 4H). MS (ESI) m/z 1658.3 [M + 2H]⁺ |
| IG-L-78 | 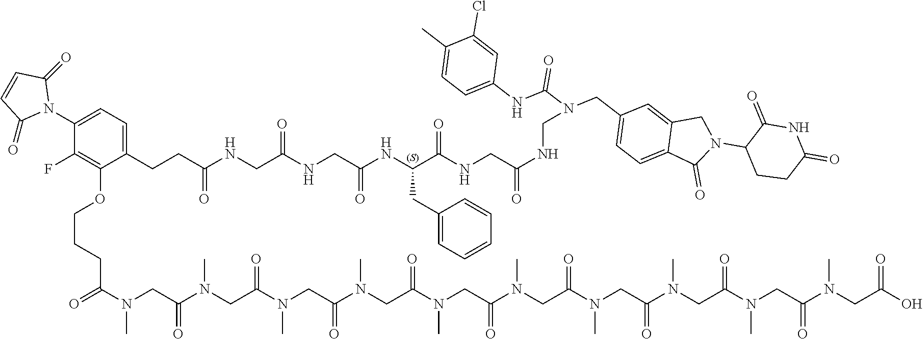 | ¹H NMR (400 MHz, DMSO-d₆) δ = 10.98 (s, 1H), 9.79-9.57 (m, 1H), 9.20-8.92 (m, 1H), 8.66-8.39 (m, 1H), 8.21 (br s, 2H), 8.07 (br d, J = 5.2 Hz, 1H), 7.71 (d, J = 8.0 Hz, 1H), 7.62 (d, J = 2.0 Hz, 1H), 7.53 (s, 1H), 7.46 (br d, J = 8.0 Hz, 1H), 7.29-7.16 (m, 9H), 7.13-7.09 (m, 1H), 7.06-6.99 (m, 1H), 5.10 (dd, J = 5.2, 13.2 Hz, 1H), 4.74- 4.58 (m, 4H), 4.53-4.41 (m, 2H), 4.37-4.16 (m, 10H), 4.13-3.87 (m, 13H), 3.80 (br d, J = 5.2 Hz, 2H), 3.74-3.57 (m, 4H), 3.08 (br dd, J = 4.0, 13.6 Hz, 1H), 3.01-2.68 (m, 36H), 2.61 (br s, 1H), 2.47-2.44 (m, 2H), 2.37 (br dd, J = 4.4, 13.2 Hz, 1H), 2.23 (s, 3H), 2.02-1.88 (m, 3H). MS (ESI) m/z 1848.1 [M + 3H]⁺ |
| IG-L-79 | 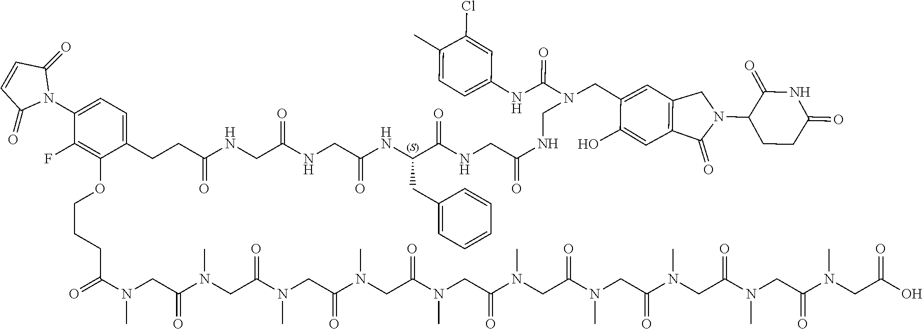 | ¹H NMR (400 MHz, DMSO-d₆) δ = 10.96 (s, 1H), 9.86-9.53 (m, 1H), 9.23-8.88(m, 1H), 8.59-8.39 (m, 1H), 8.28-8.12 (m, 2H), 8.11-8.02 (m, 1H), 7.61 (s, 1H), 7.33-7.14 (m, 10H), 7.12 (br s, 2H), 7.03 (br t, J = 7.6 Hz, 1H), 5.05 (br dd, J = 5.2, 12.8 Hz, 1H), 4.78-4.66 (m, 2H), 4.59-4.48 (m, 3H), 4.39- 3.88 (m, 26H), 3.82-3.68 (m, 5H), 3.63-3.57 (m, 1H), 3.17 (d, J = 4.8 Hz, 1H), 3.09-3.04 (m, 1H), 2.99-2.70 (m, 36H), 2.59 (br s, 1H), 2.46-2.44 (m, 1H), 2.23 (s, 3H), 2.00- 1.87 (m, 3H). LC-MS (ESI) m/z 931.7 [M/2 + H]⁺ |
| IG-L-80 | 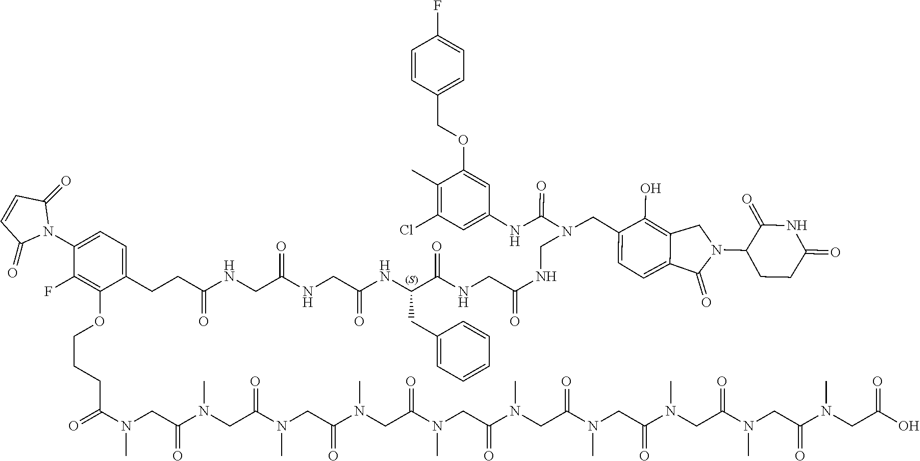 | ¹H NMR (400 MHz, DMSO-d₆) δ = 12.75 (s, 1H), 10.98 (s, 1H), 10.38 (br s, 1H), 9.83 (br s, 1H), 9.06 (br s, 1H), 8.48 (br s, 1H), 8.26-8.13 (m, 2H), 8.11-8.01 (m, 1H), 7.48 (dd, J = 5.8, 8.4 Hz, 2H), 7.38 (br d, J = 8.4 Hz, 1H), 7.25-7.15 (m, 12H), 7.13- 7.08 (m, 1H), 7.06-6.99 (m, 1H), 5.14-5.06 (m, 1H), 5.01 (s, 2H), 4.68 (br d, J = 4.0 Hz, 2H), 4.60-4.48 (m, 3H), 4.37-4.17 (m, 11H), 4.13-3.88 (m, 13H), 3.84 (br d, J = 4.4 Hz, 2H), 3.78-3.58 (m, 4H), 3.08 (br dd, J = 3.6, 13.7 Hz, 1H), 2.99-2.68 (m, 36H), 2.59 (br d, J = 15.6 Hz, 1H), 2.45- 2.35 (m, 2H), 2.29-2.18 (m, 1H), 2.13 (s, 3H), 2.03-1.97 (m, 1H), 1.95-1.86 (m, 2H). LC-MS (ESI) m/z 993.9 [M/2 + H]⁺ |

### Example L-40: Synthesis of 34-(6-((7S)-7-benzyl-1-(3-(4-hydroxy-1-oxo-5-((3-(3-(1-(trifluoromethyl)cyclopropyl)phenyl)ureido)methyl)isoindolin-2-yl)-2,6-dioxopiperidin-1-yl)-3,6,9,12,15-pentaoxo-2,5,8,11,14-pentaazaheptadecan-17-yl)-3-(2,5-dioxo-2,5-dihydro-1H-pyrrol-1-yl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, IG-L-40

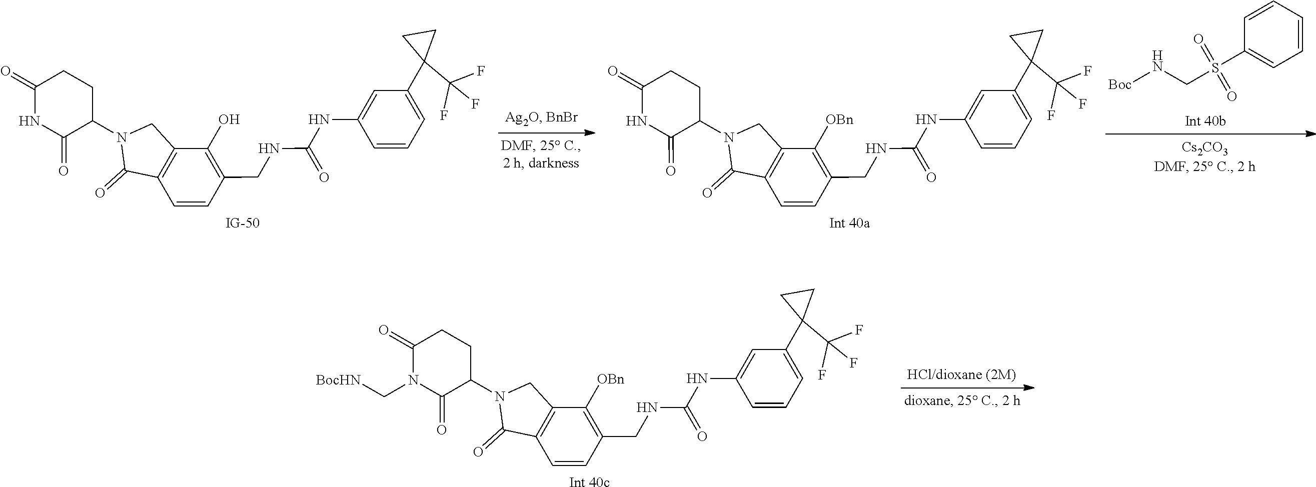

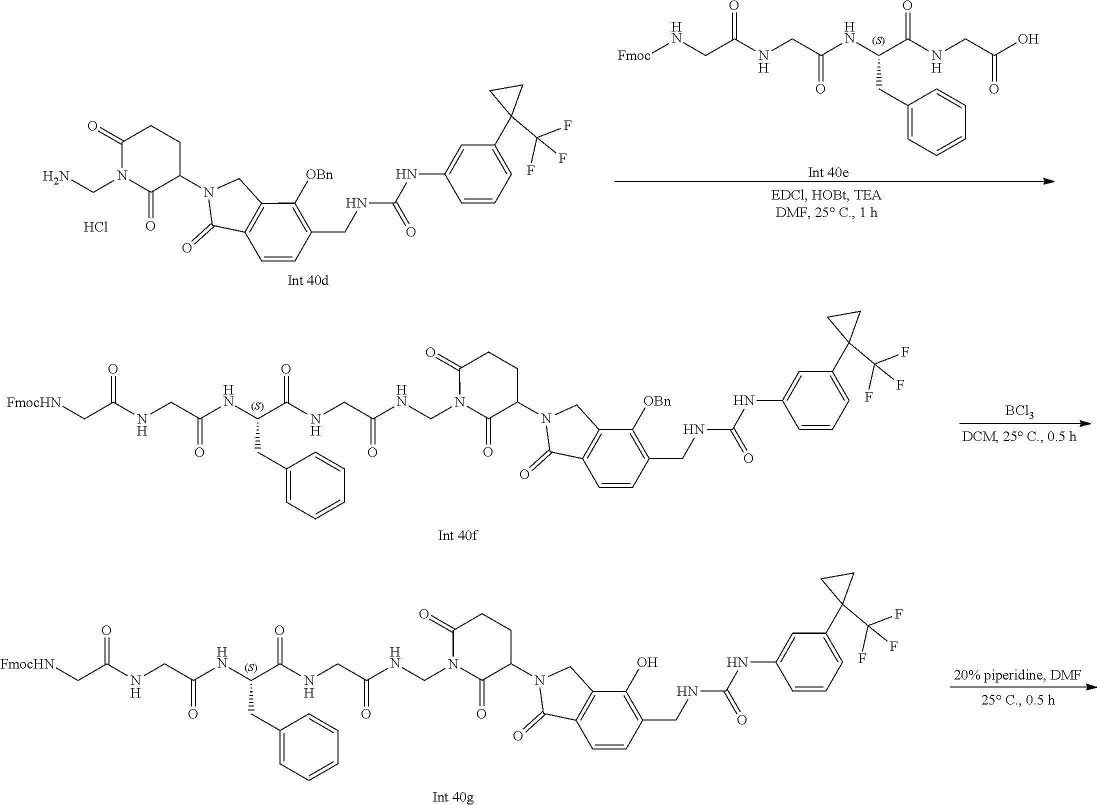

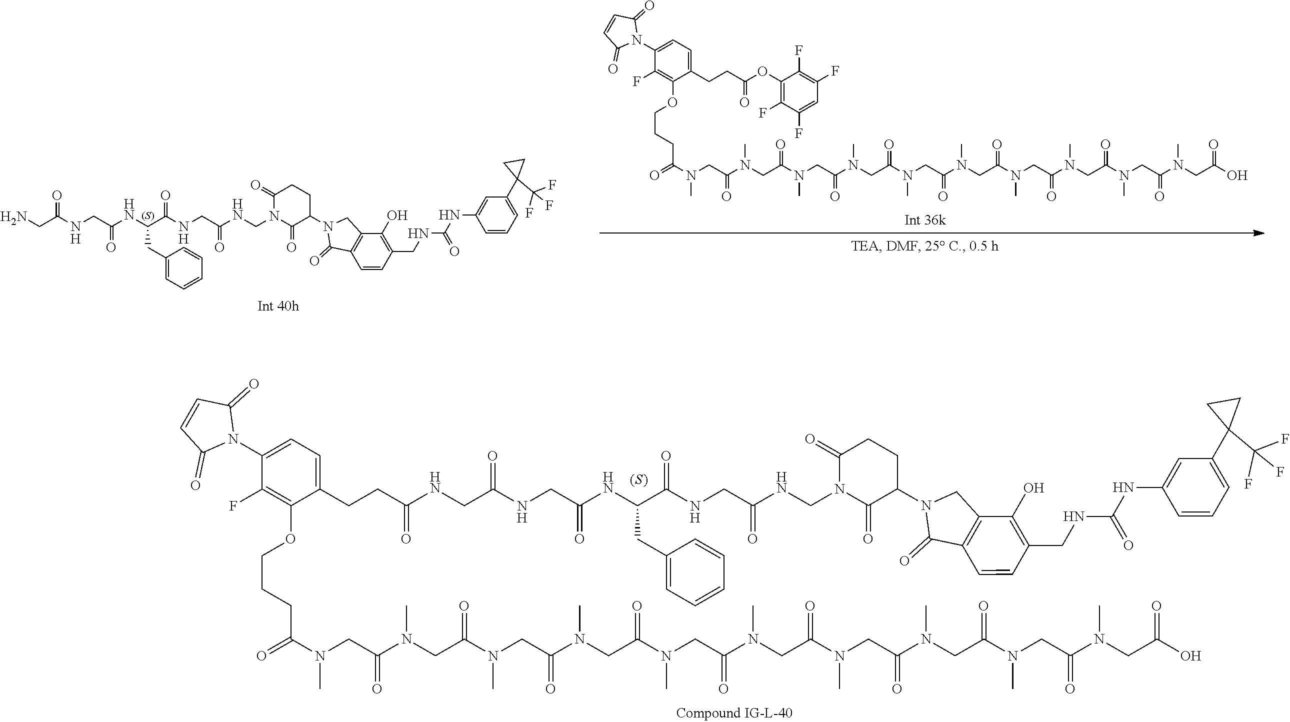

### Step A. Preparation of Int 40a

To a solution of 1-((2-(2,6-dioxopiperidin-3-yl)-4-hydroxy-1-oxoisoindolin-5-yl)methyl)-3-(3-(1-(trifluoromethyl)cyclopropyl)phenyl)urea, IG-50 (1.70 g, 3.29 mmol, 1.00 eq) in dimethyl formamide (20.0 mL) were added silver oxide (3.81 g, 16.5 mmol, 5.00 eq) and benzyl bromide (1.41 g, 8.23 mmol, 977 μL, 2.50 eq). The mixture was stirred at 25° C. for 2 h under darkness. The reaction mixture was filtered to give filtrate. The filtrate was purified by reversed-phase HPLC (0.1% formic acid) and lyophilized to give 1-((4-(benzyloxy)-2-(2,6-dioxopiperidin-3-yl)-1-oxoisoindolin-5-yl)methyl)-3-(3-(1-(trifluoromethyl)cyclopropyl)phenyl)urea, Int 40a (700 mg, 1.15 mmol, 35% yield) as a yellow solid. ¹H NMR (400 MHz, DMSO-d₆) δ=11.01 (s, 1H), 8.77 (s, 1H), 7.60 (s, 1H), 7.54 (br d, J=7.2 Hz, 2H), 7.48-7.35 (m, 6H), 7.23-7.20 (m, 1H), 6.99 (br d, J=7.6 Hz, 1H), 6.61 (br t, J=5.6 Hz, 1H), 5.23 (s, 2H), 5.15-5.10 (m, 1H), 4.70-4.65 (m, 1H), 4.56-4.49 (m, 1H), 4.47-4.37 (m, 2H), 2.98-2.90 (m, 1H), 2.62 (br d, J=17.2 Hz, 1H), 2.49-2.42 (m, 1H), 2.03-1.97 (m, 1H), 1.33-1.28 (m, 2H), 1.07 (br s, 2H). MS (ESI) m/z 607.2 [M+H]⁺

### Step B. Preparation of Int 40b

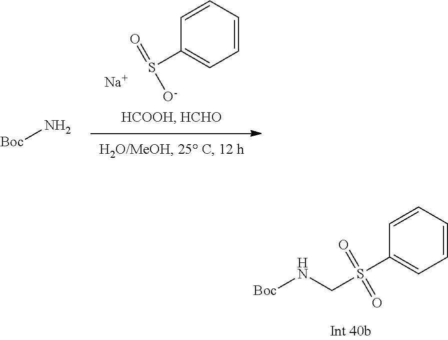 [SMILES: [Na+] ; O=S([O-])c1ccccc1 ; CC(C)(C)OC(N)=O ; CC(C)(C)OC(=O)NCS(=O)(=O)c1ccccc1]

To a solution of tert-butyl carbamate (3.00 g, 25.6 mmol, 1.00 eq) in water (40.0 mL) and methanol (40.0 mL) were added sodium benzene sulfinate (8.41 g, 51.2 mmol, 2.00 eq), carbinol (20.0 mL), formic acid (3.08 g, 64.0 mmol, 2.50 eq) and formaldehyde (1.54 g, 51.2 mmol, 1.41 mL, 2.00 eq). The mixture was stirred at 25° C. for 12 h. The reaction mixture was concentrated under reduced pressure to give a residue. The residue was purified by column chromatography on silica gel (petroleum ether/ethyl acetate=3/1). The desired fraction was concentrated under reduced pressure to give tert-butyl ((phenylsulfonyl)methyl)carbamate, Int 40b (4.50 g, 16.4 mmol, 64% yield, 99% purity) as a white solid. ¹H NMR (400 MHz, DMSO-d₆) δ=8.06 (br t, J=6.8 Hz, 1H), 7.82 (br d, J=7.6 Hz, 2H), 7.75-7.70 (m, 1H), 7.66-7.60 (m, 2H), 4.50 (d, J=6.8 Hz, 2H), 1.22 (s, 9H). MS (ESI) m/z 294.0 [M+Na]⁺

### Step C. Preparation of Int 40c

To a solution of 1-((4-(benzyloxy)-2-(2,6-dioxopiperidin-3-yl)-1-oxoisoindolin-5-yl)methyl)-3-(3-(1-(trifluoromethyl)cyclopropyl)phenyl)urea, Int 40a (450 mg, 742 μmol, 1.00 eq) in dimethyl formamide (10.0 mL) were added tert-butyl ((phenylsulfonyl)methyl)carbamate, Int 40b (221 mg, 816 μmol, 1.10 eq) and cesium carbonate (483 mg, 1.48 mmol, 2.00 eq). The mixture was stirred at 25° C. for 2 h. The reaction mixture was filtered to give filtrate. The filtrate was purified by reversed-phase HPLC (0.10% formic acid) and lyophilized to give tert-butyl ((3-(4-(benzyloxy)-1-oxo-5-((3-(3-(1-(trifluoromethyl)cyclopropyl)phenyl)ureido)methyl) isoindolin-2-yl)-2,6-dioxopiperidin-1-yl)methyl)carbamate, Int 40c (300 mg, 408 μmol, 55% yield) as a white solid. ¹H NMR (400 MHz, DMSO-d₆) δ=8.77 (s, 1H), 7.60 (br s, 1H), 7.52 (br d, J=7.2 Hz, 2H), 7.49-7.33 (m, 6H), 7.23 (br d, J=7.6 Hz, 1H), 6.99 (br d, J=7.2 Hz, 2H), 6.61 (br t, J=5.6 Hz, 1H), 5.27-5.16 (m, 3H), 4.98 (br dd, J=5.6, 12.4 Hz, 1H), 4.91-4.84 (m, 1H), 4.67 (br d, J=17.2 Hz, 1H), 4.53-4.37 (m, 3H), 3.05-2.94 (m, 1H), 2.78 (br d, J=17.2 Hz, 1H), 2.43-2.33 (m, 1H), 2.06-2.00 (m, 1H), 1.35 (s, 9H), 1.33-1.27 (m, 2H), 1.07 (br s, 2H). MS (ESI) m/z 680.2 [M+H-56]⁺

### Step D. Preparation of Int 40d

To a solution of tert-butyl ((3-(4-(benzyloxy)-1-oxo-5-((3-(3-(1-(trifluoromethyl)cyclopropyl)phenyl)ureido) methyl)isoindolin-2-yl)-2,6-dioxopiperidin-1-yl)methyl)carbamate, Int 40c (300 mg, 408 μmol, 1.00 eq) in dioxane (2.00 mL) was added hydrochloric acid/dioxane (10.0 mL). The mixture was stirred at 25° C. for 2 h. The reaction mixture was concentrated under reduced pressure to give 1-((2-(1-(aminomethyl)-2,6-dioxopiperidin-3-yl)-4-(benzyloxy)-1-oxoisoindolin-5-yl)methyl)-3-(3-(1-(trifluoromethyl)cyclopropyl)phenyl)urea, Int 40d (250 mg, crude) as white solid. MS (ESI) m/z 636.3 [M+H]⁺

### Step E. Preparation of Int 40e

To a solution of (((9H-fluoren-9-yl)methoxy)carbonyl)glycine (15.0 g, 50.5 mmol, 1.00 eq) in dichloromethane (300 mL) was added 2-Chlorotritylchlorideresin (60.0 g) and N,N-diisopropylethylamine (16.3 g, 126 mmol, 22.0 mL, 2.50 eq). Then deprotection and acid-amine condensation in turn by solid-phase synthesis. (((9H-fluoren-9-yl)methoxy)carbonyl)-L-phenylalanine (29.0 g, 74.9 mmol, 1.50 eq) and (((9H-fluoren-9-yl)methoxy)carbonyl)glycylglycine (9.50 g, 46.3 mmol, 1.00 eq) were be used for the acid-amine condensation. Condition of acid-amine condensation: 0-(7-Azabenzotriazol-1-yl)-N,N,N′,N′-tetramethyluronium Hexafluorophosphate (1.80 eq), N,N-diisopropylethylamine (3.00 eq), dimethyl formamide (150 mL), 25° C. for 2 h. Condition of deprotection: a. dimethyl formamide (100 mL)/piperidine (100 mL), 25° C. for 5 min; b. dichloromethane (160 mL)/hexafluoroisopropanol (40.0 mL), 25° C. for 1 h. to give (((9H-fluoren-9-yl)methoxy)carbonyl)glycylglycyl-L-phenylalanylglycine, Int 40e (13.0 g, 23.3 mmol, 50% yield) as a white solid. MS (ESI) m/z 559.2 [M+H]⁺

### Step F. Preparation of Int 40f

To a solution of 1-((2-(1-(aminomethyl)-2,6-dioxopiperidin-3-yl)-4-(benzyloxy)-1-oxoisoindolin-5-yl)methyl)-3-(3-(1-(trifluoromethyl)cyclopropyl)phenyl)urea, Int 40d (250 mg, 393 gmol, 1.00 eq) in dimethyl formamide (3.00 mL) were added (((9H-fluoren-9-yl)methoxy)carbonyl)glycylglycyl-L-phenylalanylglycine, Int 53e (220 mg, 393 gmol, 1.00 eq), triethylamine (79.6 mg, 787 gmol, 109 μL, 2.00 eq) 3-(ethyliminomethylideneamino)propyl-dimethylazanium;chloride (113 mg, 590 gmol, 1.50 eq) and 1H-benzo[d][1,2,3]triazol-1-ol (79.7 mg, 590 μmol, 1.50 eq). The mixture was stirred at 25° C. for 1 h. The reaction mixture was filtered to give a filtrate. The filtrate was purified by reversed-phase HPLC (0.1% formic acid) and lyophilized to give (9H-fluoren-9-yl)methyl ((7S)-7-benzyl-1-(3-(4-(benzyloxy)-1-oxo-5-((3-(3-(1-(trifluoromethyl)cyclopropyl)phenyl)ureido)methyl)isoindolin-2-yl)-2,6-dioxopiperidin-1-yl)-3,6,9,12-tetraoxo-2,5,8,11-tetraazatridecan-13-yl)carbamate, Int 40f (240 mg, 204 gmol, 52% yield) as a white solid. ¹H NMR (400 MHz, DMSO-d₆) δ=8.95 (s, 1H), 8.38 (br t, J=5.2 Hz, 1H), 8.32-8.21 (m, 2H), 8.13 (br d, J=4.4 Hz, 1H), 8.01 (d, J=7.6 Hz, 2H), 7.83 (br d, J=7.2 Hz, 2H), 7.74-7.69 (m, 2H), 7.65 (br d, J=7.2 Hz, 2H), 7.63-7.58 (m, 2H), 7.57-7.50 (m, 5H), 7.48-7.42 (m, 3H), 7.38-7.33 (m, 5H), 7.30 (br d, J=6.4 Hz, 1H), 7.12 (d, J=7.6 Hz, 1H), 6.78 (br t, J=5.6 Hz, 1H), 5.37-5.29 (m, 4H), 5.12 (td, J=6.0, 12.4 Hz, 1H), 4.85-4.78 (m, 1H), 4.69-4.60 (m, 2H), 4.55 (br d, J=5.2 Hz, 2H), 4.46-4.38 (m, 2H), 4.36 (br d, J=6.4 Hz, 1H), 3.90-3.72 (m, 6H), 3.19-3.11 (m, 2H), 2.97-2.86 (m, 2H), 2.60-2.50 (m, 1H), 2.21-2.13 (m, 1H), 1.47-1.39 (m, 2H), 1.20 (br s, 2H). MS (ESI) m/z 1176.4 [M+H]⁺

### Step G. Preparation of Int 40g

To a solution of (9H-fluoren-9-yl)methyl ((7S)-7-benzyl-1-(3-(4-(benzyloxy)-1-oxo-5-((3-(3-(1-(trifluoromethyl)cyclopropyl)phenyl) ureido)methyl)isoindolin-2-yl)-2,6-dioxopiperidin-1-yl)-3,6,9,12-tetraoxo-2,5,8,11-tetraazatridecan-13-yl)carbamate, Int 40f (240 mg, 204 gmol, 1.00 eq) in dichloromethane (5.00 mL) was added boron trichloride (2 M, 1.02 mL, 10.0 eq) at 0° C. The mixture was stirred at 25° C. for 0.5 h. The reaction mixture was quenched by addition water (5 mL) at 0° C. and filtered to give (9H-fluoren-9-yl)methyl ((7S)-7-benzyl-1-(3-(4-hydroxy-1-oxo-5-((3-(3-(1-(trifluoromethyl)cyclopropyl) phenyl)ureido) methyl)isoindolin-2-yl)-2,6-dioxopiperidin-1-yl)-3,6,9,12-tetraoxo-2,5,8,11-tetraazatridecan-13-yl)carbamate, Int 40g (200 mg, crude) as white solid. MS (ESI) m/z 1086.4 [M+H]⁺

### Step H. Preparation of Int 40h

To a solution of (9H-fluoren-9-yl)methyl ((7S)-7-benzyl-1-(3-(4-hydroxy-1-oxo-5-((3-(3-(1-(trifluoromethyl)cyclopropyl)phenyl)ureido)methyl)isoindolin-2-yl)-2,6-dioxopiperidin-1-yl)-3,6,9,12-tetraoxo-2,5,8,11-tetraazatridecan-13-yl)carbamate, Int 40g (200 mg, 184 μmol, 1.00 eq) in dimethyl formamide (2.00 mL) was added piperidine (0.500 mL). The mixture was stirred at 25° C. for 0.5 h. The reaction mixture was filtered. The filtrate was purified by prep-HPLC (column: Phenomenex luna C18 150*25 mm*10 um; mobile phase: [water(formic acid)-acetonitrile]; gradient: 18%-48% B over 9 min) and lyophilized to give (2S)-2-(2-(2-aminoacetamido)acetamido)-N-(2-(((3-(4-hydroxy-1-oxo-5-((3-(3-(1-(trifluoromethyl) cyclopropyl)phenyl)ureido)methyl)isoindolin-2-yl)-2,6-dioxopiperidin-1-yl)methyl)amino)-2-oxoethyl)-3-phenylpropanamide, Int 40h (60.0 mg, 69.5 gmol, 38% yield) as a white solid. ¹H NMR (400 MHz, DMSO-d₆) δ=9.09 (s, 1H), 8.39-8.28 (m, 2H), 8.28-8.11 (m, 3H), 7.62 (s, 1H), 7.40 (br d, J=7.2 Hz, 2H), 7.30-7.21 (m, 7H), 7.04 (br d, J=7.2 Hz, 2H), 5.25-5.15 (m, 2H), 5.07-4.97 (m, 1H), 4.59-4.50 (m, 1H), 4.45-4.36 (m, 3H), 4.32-4.25 (m, 1H), 3.85-3.65 (m, 6H), 3.24 (s, 2H), 3.09-3.02 (m, 2H), 2.85-2.72 (m, 2H), 2.38 (br d, J=8.8 Hz, 1H), 2.12-2.06 (m, 1H), 1.38-1.32 (m, 2H), 1.11 (br s, 2H). MS (ESI) m/z 864.3 [M+H]⁺

### Step I. Preparation of Compound IG-L-40

To a solution of (2S)-2-(2-(2-aminoacetamido)acetamido)-N-(2-(((3-(4-hydroxy-1-oxo-5-((3-(3-(1-(trifluoromethyl)cyclopropyl)phenyl)ureido)methyl)isoindolin-2-yl)-2,6-dioxopiperidin-1-yl)methyl)amino)-2-oxoethyl)-3-phenylpropanamide, Int 40h (30.0 mg, 34.7 gmol, 1.00 eq) in dimethyl formamide (1.00 mL) were added triethylamine (10.5 mg, 104 gmol, 14.5 μL, 3.00 eq) and 34-(3-(2,5-dioxo-2,5-dihydro-1H-pyrrol-1-yl)-2-fluoro-6-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, Int 36k (42.5 mg, 34.7 μmol, 1.00 eq). The mixture was stirred at 25° C. for 0.5 h. The reaction mixture was filtered. The filtrate was purified by prep-HPLC (column: UniSil 3-100 C18 Ultra (150*25 mm*3 um); mobile phase: [water(formic acid)-acetonitrile]; gradient: 20%-50% B over 40 min) and lyophilized to give 34-(6-((7S)-7-benzyl-1-(3-(4-hydroxy-1-oxo-5-((3-(3-(1-(trifluoromethyl)cyclopropyl)phenyl) ureido)methyl) isoindolin-2-yl)-2,6-dioxopiperidin-1-yl)-3,6,9,12,15-pentaoxo-2,5,8,11,14-pentaazaheptadecan-17-yl)-3-(2,5-dioxo-2,5-dihydro-1H-pyrrol-1-yl)-2-fluorophenoxy)-3,6,9,12,15,18,21,24,27,30-decamethyl-4,7,10,13, 16,19,22,25, 28,31-decaoxo-3,6,9,12,15,18,21,24,27,30-decaazatetratriacontanoic acid, Compound IG-L-40 (22.93 mg, 11.8 μmol, 34% yield, 99% purity) as a white solid. ¹H NMR (400 MHz, DMSO-d₆) δ=10.62-10.01 (m, 1H), 9.15-8.89 (m, 1H), 8.30-8.01 (m, 5H), 7.58 (br s, 1H), 7.38-7.33 (m, 2H), 7.27-7.11 (m, 10H), 7.06-6.99 (m, 2H), 6.95-6.84 (m, 1H), 5.23-5.11 (m, 2H), 4.99 (dt, J=5.2, 13.2 Hz, 1H), 4.53-4.46 (m, 1H), 4.39-3.87 (m, 27H), 3.79-3.67 (m, 5H), 3.59 (br dd, J=5.4, 16.8 Hz, 1H), 3.05-2.70 (m, 38H), 2.37-2.21 (m, 2H), 2.07-2.00 (m, 1H), 1.98-1.86 (m, 2H), 1.35-1.29 (m, 2H), 1.07 (br s, 2H). MS (ESI) m/z 1922.6 [M+2H]⁺

The compounds in Table 18 were prepared in a manner similar to that described for Compound IG-L-40 using the appropriate compound as starting material.

TABLE 18

| IG-L No. | Structure | ¹H NMR/MS |
|---|---|---|
| IG-L-41 | 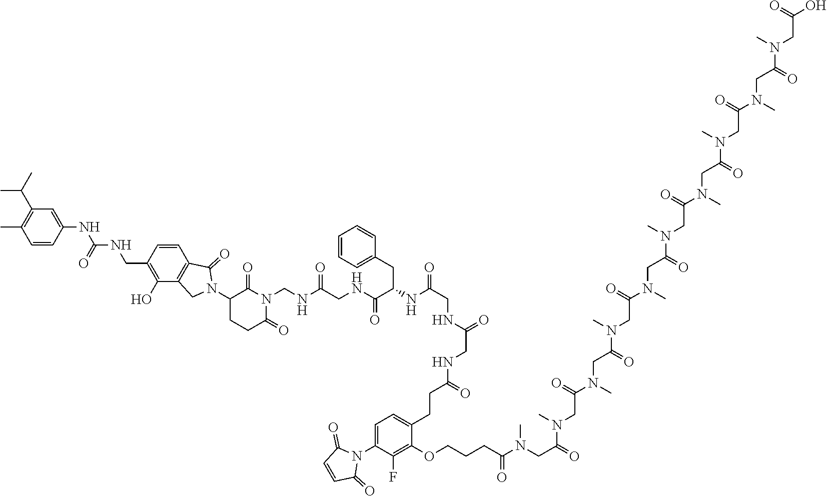 [SMILES: Cc1ccc(NC(=O)NCc2ccc3c(c2O)CN(C2CCC(=O)N(CNC(=O)CNC(=O)[C@H](Cc4ccccc4)NC(=O)CNC(=O)CNC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1C(C)C] | ¹H NMR (400 MHz, DMSO-d₆) δ = 10.39- 10.23 (m, 1H), 8.77-8.62 (m, 1H), 8.26- 8.01 (m, 5H), 7.36 (d, J = 7.6 Hz, 1H), 7.26-7.19 (m, 8H), 7.18-7.09 (m, 3H), 7.03 (br t, J = 7.2 Hz, 1H), 6.97 (d, J = 8.4 Hz, 1H), 6.81-6.68 (m, 1H), 5.23-5.09 (m, 2H), 5.04-4.92 (m, 1H), 4.53-4.46 (m, 1H), 4.37- 4.29 (m, 7H), 4.26-4.16 (m, 6H), 4.11-3.88 (m, 13H), 3.76-3.66 (m, 5H), 3.62-3.55 (m, 1H), 3.04-2.69 (m, 41H), 2.38-2.27 (m, 2H), 2.19 (s, 3H), 2.07-2.01 (m, 1H), 1.96-1.88 (m, 2H), 1.13 (d, J = 6.8 Hz, 6H). MS (ESI) m/z 1871.0 [M + H]+ |
| IG-L-46 | 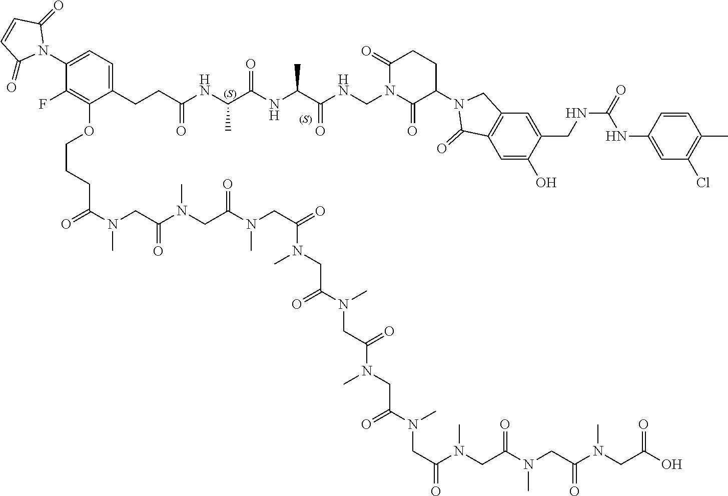 | ¹H NMR (400 MHz, DMSO-d₆) δ = 12.76 (s, 1H), 10.40-10.03 (m, 1H), 9.18-8.76 (m, 1H), 8.17-7.94 (m, 3H), 7.65 (d, J = 2.0 Hz, 1H), 7.36 (s, 1H), 7.24 (s, 2H), 7.18- 7.07 (m, 4H), 7.06-6.99 (m, 1H), 6.81-6.61 (m, 1H), 5.20-5.03 (m, 2H), 5.02-4.85 (m, 1H), 4.35-3.86 (m, 28H), 3.06-2.65 (m, 36H), 2.47-2.39 (m, 2H), 2.33-2.27 (m, 1H), 2.22 (s, 3H), 2.03-1.87 (m, 3H), 1.18- 1.10 (m, 6H). MS (ESI) m/z 1686.8, 844.1 [M + 2H, M/2 + 2H]⁺ |
| IG-L-47 | 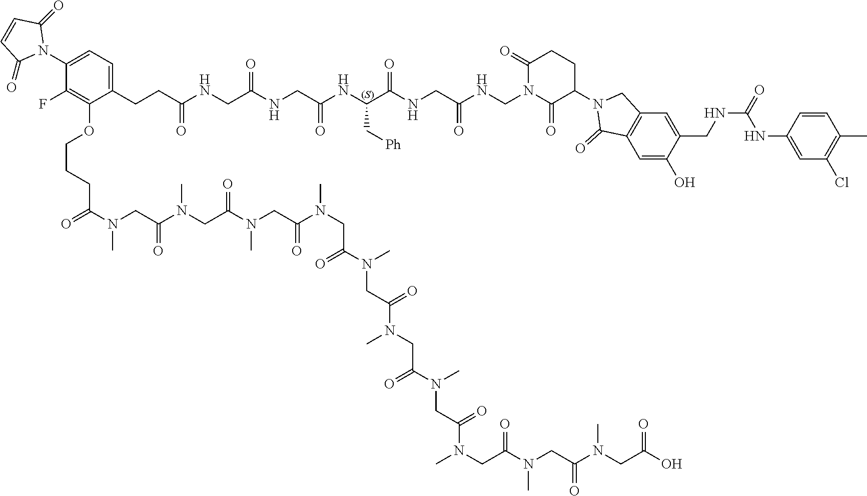 | ¹H NMR (400 MHz, DMSO-d₆) δ = 12.77 (s, 1H), 10.15 (br s, 1H), 8.81 (br s, 1H), 8.28-8.02 (m, 5H), 7.65 (d, J = 2.0 Hz, 1H), 7.35 (s, 1H), 7.26-7.21 (m, 6H), 7.18-7.08 (m, 5H), 7.03 (br t, J = 7.6 Hz, 1H), 6.74- 6.62 (m, 1H), 5.23-5.09 (m, 2H), 5.00-4.89 m, 1H), 4.53-4.42 (m, 1H), 4.38-4.14 (m, 14H), 4.13-3.87 (m, 13H), 3.78-3.72 (m, 1H), 3.69 (br d, J = 6.0 Hz, 4H), 3.62-3.55 (m, 1H), 3.04-2.70 (m, 38H), 2.47-2.43 (m, 1H), 2.33-2.29 (m, 1H), 2.22 (s, 3H), 2.03- 1.87 (m, 3H). MS (ESI) m/z 1863.1, 932.0 [M + 3H, M/2 + 2H]⁺ |
| IG-L-48 | 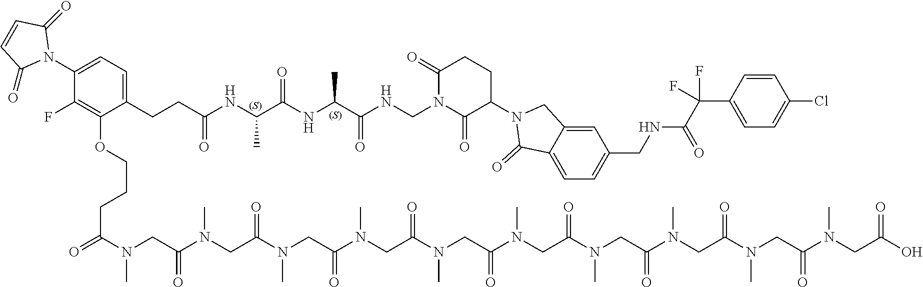 | ¹H NMR (400 MHz, DMSO-d₆) δ = 9.72 (br dd, J = 4.4 13.2 Hz, 1H), 8.22-8.01 (m, 3H), 7.69 (br d, J = 7.6 Hz, 1H), 7.62 (s, 4H), 7.42 (s, 1H), 7.36 (br d, J = 8.0 Hz, 1H), 7.24 (s, 2H), 7.15-7.07 (m, 1H), 7.02 (q, J = 6.8 Hz, 1H), 5.22-5.10 (m, 2H), 5.01-4.88 (m, 1H), 4.50-4.40 (m, 4H), 4.34-4.19 (m, 12H), 4.10-3.92 (m, 12H), 2.96-2.87 (m, 14H), 2.85-2.70 (m, 22H), 2.45 (br dd, J = 4.0, 6.4 Hz, 2H), 2.07-1.86 (m, 4H), 1.17-1.13 (m, 6H). MS (ESI) m/z 846.6 [M/2 + 2H]⁺ |
| IG-L-49 | 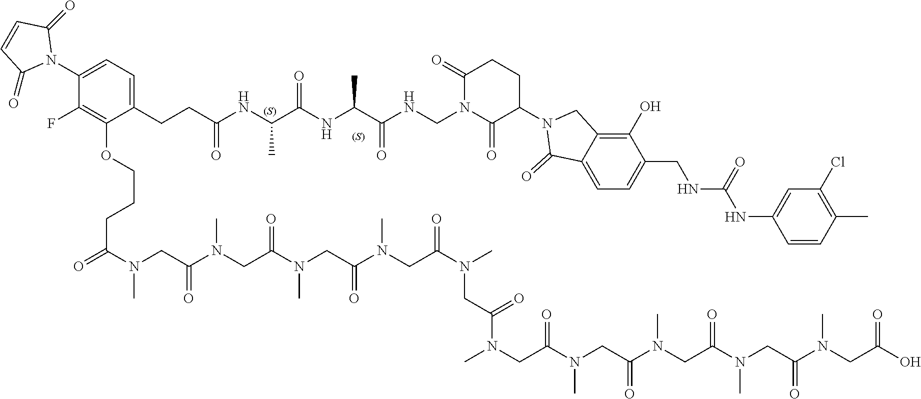 | ¹H NMR (400 MHz, DMSO-d₆) δ = 10.32- 10.03 (m, 1H), 9.02-8.86 (m, 1H), 8.18- 7.95 (m, 3H), 7.62 (s, 1H), 7.37-7.31 (m, 1H), 7.24 (s, 2H), 7.22-7.15 (m, 2H), 7.12 (br d, J = 7.6 Hz, 2H), 7.06-6.98 (m, 1H), 6.97-6.78 (m, 1H), 5.28-4.81 (m, 3H), 4.39- 4.14 (m, 16H), 4.11-3.89 (m, 12H), 3.02- 2.64 (m, 36H), 2.47-2.41 (m, 2H), 2.33- 2.30 (m, 1H), 2.23 (s, 3H), 2.06-2.00 (m, 1H), 1.96-1.87 (m, 2H), 1.15 (br d, J = 6.8 Hz, 6H). MS (ESI) m/z 1685.8 [M + H]⁺ |
| IG-L-50 | 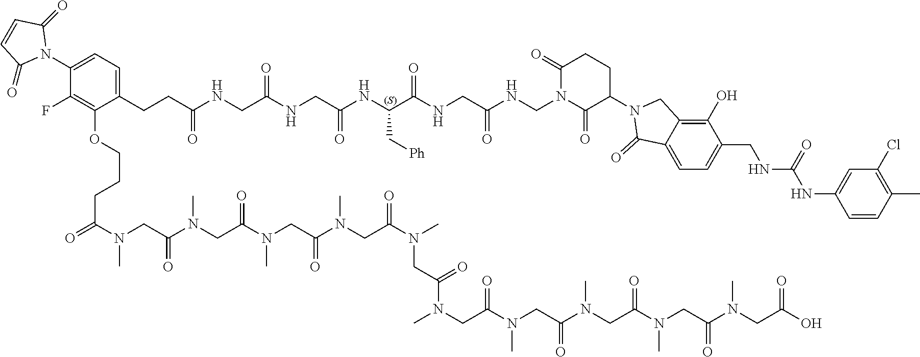 | ¹H NMR (400 MHz, DMSO-d₆) δ = 12.77 (s, 1H), 10.15 (br s, 1H), 8.90 (br s, 1H), 8.28-8.02 (m, 5H), 7.62 (s, 1H), 7.35 (br d, J = 7.6 Hz, 1H), 7.27-7.09 (m, 11H), 7.07- 7.00 (m, 1H), 6.86 (br s, 1H), 5.24-5.10 (m, 2H), 5.04-4.94 (m, 1H), 4.51-4.44 (m, 1H), 4.40-4.17 (m, 14H), 4.13-3.87 (m, 13H), 3.78-3.66 (m, 5H), 3.59 (br dd, J = 4.8, 16.8 Hz, 1H), 3.05-2.70 (m, 38H), 2.48-2.44 (m, 1H), 2.35-2.29 (m, 1H), 2.23 (s, 3H), 2.09- 2.01 (m, 1H), 2.00-1.84 (m, 2H). MS (ESI) m/z 1863.1 [M + H]⁺ |
| IG-L-54 | 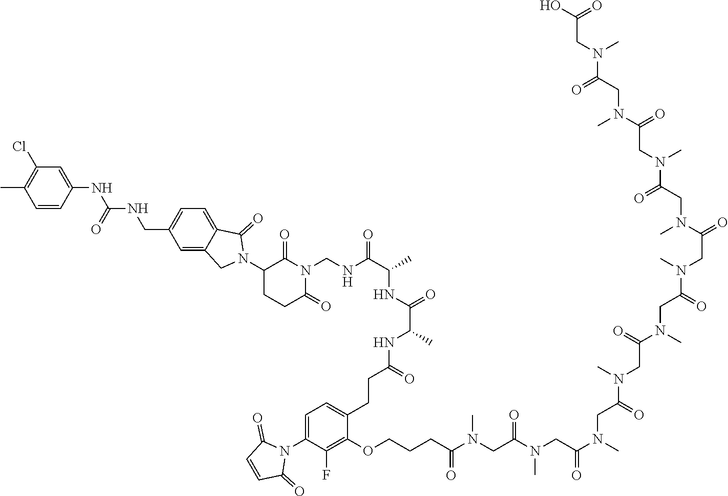 [SMILES: Cc1ccc(NC(=O)NCc2ccc3c(c2)CN(C2CCC(=O)N(CNC(=O)[C@H](C)NC(=O)[C@H](C)NC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl] | ¹H NMR (400 MHz, DMSO-d₆) δ = 8.73 (s, 1H), 8.15-7.93 (m, 3H), 7.73-7.68 (m, 1H), 7.65 (d, J = 2.0 Hz, 1H), 7.52 (s, 1H), 7.44 (d, J = 7.6 Hz, 1H), 7.24 (s, 2H), 7.20-707 (m, 3H), 7.06-6.96 (m, 1H), 6.78 (t, J = 6.0 Hz, 1H), 5.24-4.85 (m, 3H), 4.49-3.87 (m, 26H), 3.05-2.65 (m, 34H), 2.47-2.38 (m, 2H), 2.36-2.27 (m, 2H), 2.22 (s, 3H), 2.08- 1.85 (m, 3H), 1.15 (d, J = 6.4 Hz, 6H) MS (ESI) m/z 1669.7[M + H]⁺ |
| IG-L-58 | 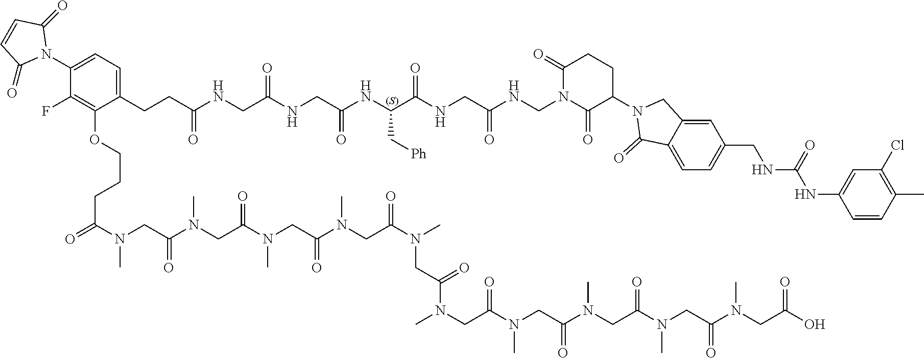 | ¹H NMR (400 MHz, DMSO-d₆) δ = 9.00- 8.76 (m, 1H), 8.27-8.21(m, 1H), 8.19-8.08 (m, 3H), 8.07-7.99 (m, 1H), 7.71-7.66 (m, 2H), 7.50 (s, 1H), 7.44 (br d, J = 8.0 Hz, 1H), 7.26-7.13 (m, 11H), 6.89 (br d, J = 8.0 Hz, 1H), 6.78 (br s, 1H), 5.25-5.11 (m, 2H), 5.02-4.90 (m, 1H), 4.48-4.20 (m, 14H), 4.10-3.88 (m, 13H), 3.76-3.67 (m, 5H), 3.63-3.56 (m, 1H), 3.04-2.73 (m, 38H), 2.43 (br d, J = 8.4 Hz, 2H), 2.32 (br d, J = 2.0 Hz, 1H), 2.22 (s, 3H), 2.06-1.90 (m, 3H). MS (ESI) m/z 1829.5 [M + 3H]⁺ |

### Example L-44: Synthesis of 34-(3-(2,5-dioxo-2,5-dihydro-1H-pyrrol-1-yl)-6-(3-(((2S)-1-(((2S)-1-((4-((((2-((((2-(2,6-dioxopiperidin-3-yl)-5-((3-(3-isopropyl-4-methylphenyl)ureido)methyl)-1-oxoisoindolin-4-yl)oxy)carbonyl)(methyl)amino)ethyl)(methoxy)carbamoyl)oxy)methyl)phenyl)amino)-1-oxopropan-2-yl)amino)-1-oxopropan-2-yl)amino)-3-oxopropyl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, IG-L-44

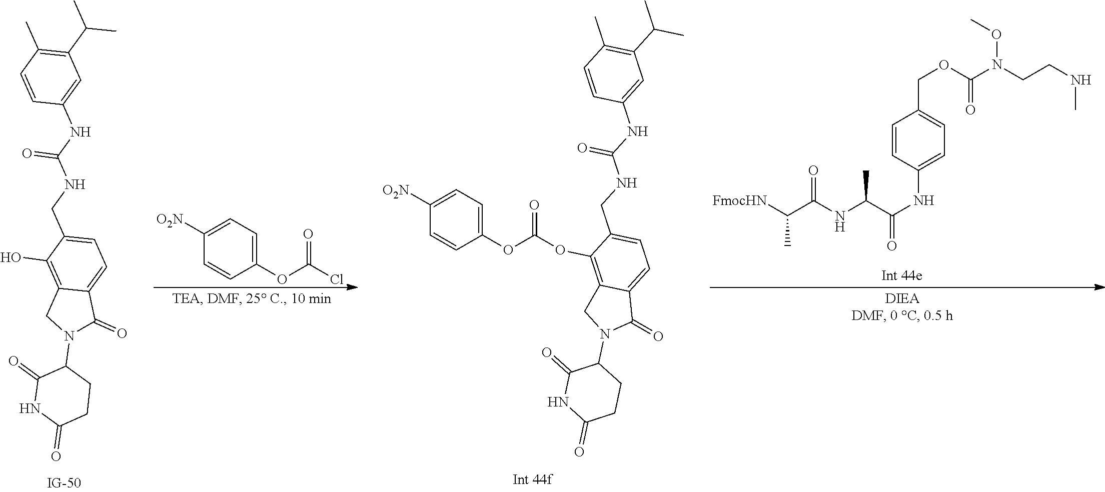

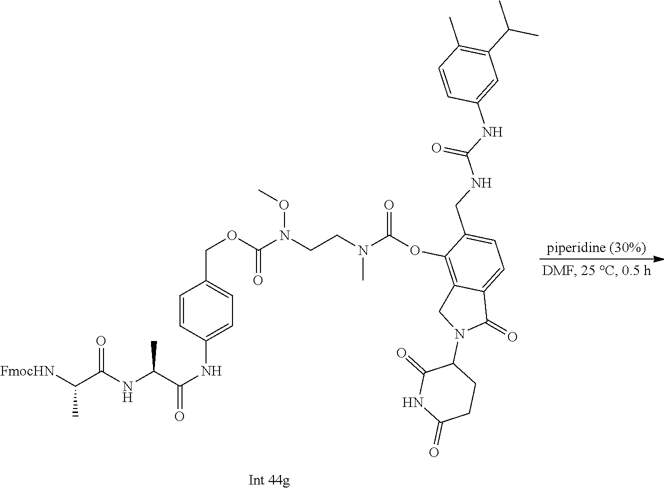

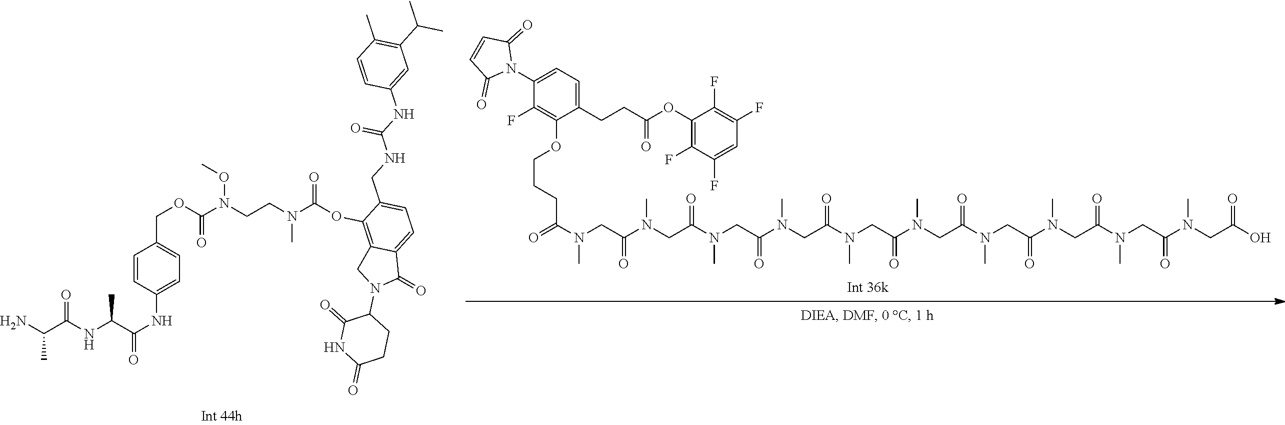 [SMILES: CON(CCN(C)C(=O)Oc1c(CNC(=O)Nc2ccc(C)c(C(C)C)c2)ccc2c1CN(C1CCC(=O)NC1=O)C2=O)C(=O)OCc1ccc(NC(=O)[C@H](C)NC(=O)[C@H](C)N)cc1 ; CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCCOc1c(CCC(=O)Oc2c(F)c(F)cc(F)c2F)ccc(N2C(=O)C=CC2=O)c1F]

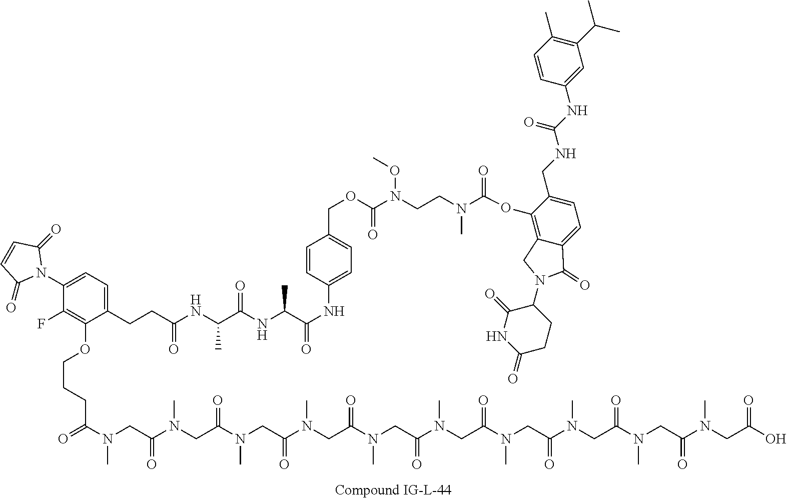 [SMILES: CON(CCN(C)C(=O)Oc1c(CNC(=O)Nc2ccc(C)c(C(C)C)c2)ccc2c1CN(C1CCC(=O)NC1=O)C2=O)C(=O)OCc1ccc(NC(=O)[C@H](C)NC(=O)[C@H](C)NC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc1]

### Step A. Preparation of Int 54a

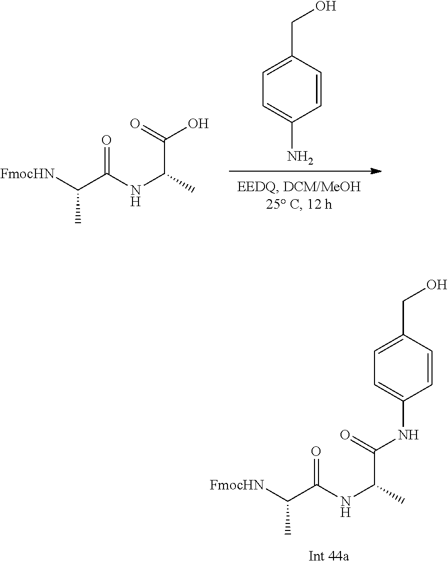

To a solution of (((9H-fluoren-9-yl)methoxy)carbonyl)-L-alanyl-L-alanine (20.0 g, 52.3 mmol, 1.00 eq) and (4-aminophenyl)methanol (7.73 g, 62.8 mmol, 1.20 eq) in dichloromethane (300 mL) and methanol (300 mL) was added (9H-fluoren-9-yl)methyl ((S)-3-methyl-1-(((S)-1-((4-((((4-nitrophenoxy)carbonyl)oxy) methyl)phenyl)amino)-1-oxo-5-ureidopentan-2-yl)amino)-1-oxobutan-2-yl)carbamate (38.8 g, 157 mmol, 3.00 eq), the mixture was stirred at 25° C. for 12 h. The mixture concentrated in vacuum to give a residue. The residue was triturated with tetrahydrofuran (300 mL) and filtered to afford (9H-fluoren-9-yl)methyl ((S)-1-(((S)-1-((4-(hydroxymethyl)phenyl)amino)-1-oxopropan-2-yl)amino)-1-oxopropan-2-yl)carbamate, Int 44a (18.0 g, 36.9 mmol, 71% yield) as a yellow solid. ¹H NMR (400 MHz, DMSO-d₆) δ=9.89 (s, 1H), 8.11 (br d, J=7.3 Hz, 1H), 7.90 (d, J=7.5 Hz, 2H), 7.73 (brt, J=8.1 Hz, 2H), 7.59-7.53 (m, 3H), 7.42 (br t, J=7.4 Hz, 2H), 7.37-7.31 (m, 2H), 7.24 (d, J=8.4 Hz, 2H), 5.11 (t, J=5.7 Hz, 1H), 4.48-4.39 (m, 3H), 4.30-4.21 (m, 3H), 4.15-4.06 (m, 1H), 1.31 (d, J=7.1 Hz, 3H), 1.25 (d, J=7.1 Hz, 3H).

### Step B. Preparation of Int 44b

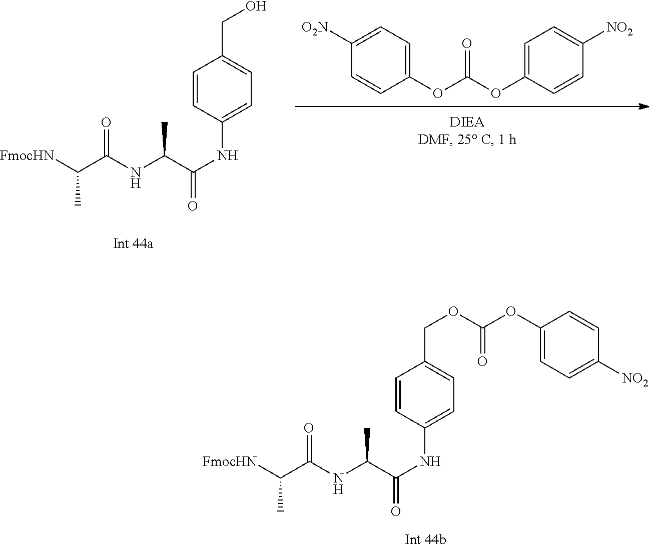

To a solution of (9H-fluoren-9-yl)methyl ((S)-1-(((S)-1-((4-(hydroxymethyl)phenyl)amino)-1-oxopropan-2-yl)amino)-1-oxopropan-2-yl)carbamate Int 44a (4.20 g, 8.61 mmol, 1.00 eq) and bis(4-nitrophenyl) carbonate (2.62 g, 8.61 mmol, 1.00 eq) in N,N-dimethylformamide (20.0 mL) was added diisopropylethylamine (3.34 g, 25.8 mmol, 4.50 mL, 3.00 eq). The mixture was stirred at 25° C. for 1 h. The mixture was concentrated under reduced pressure to give a residue. The residue was purified by reversed-phase column (C18, 220 g; condition: water/acetonitrile=1/0-0/1, 0.1% formic acid) and lyophilized to afford (9H-fluoren-9-yl)methyl ((S)-1-(((S)-1-((4-((((4-nitrophenoxy)carbonyl)oxy)methyl)phenyl)amino)-1-oxopropan-2-yl)amino)-1-oxopropan-2-yl)carbamate Int 44b (3.15 g, 4.83 mmol, 56% yield) as a white solid. ¹H NMR (400 MHz, DMSO-d₆) δ=10.04 (s, 1H), 8.38-8.24 (m, 2H), 8.13 (br d, J=7.2 Hz, 1H), 7.88 (d, J=7.2 Hz, 2H), 7.72 (br t, J=7.6 Hz, 2H), 7.64 (br d, J=8.4 Hz, 2H), 7.59-7.52 (m, 3H), 7.44-7.38 (m, 4H), 7.35-7.29 (m, 2H), 5.24 (s, 2H), 4.46-4.34 (m, 1H), 4.30-4.18 (m, 3H), 4.12-4.06 (m, 1H), 1.31 (d, J=7.2 Hz, 3H), 1.23 (br d, J=7.2 Hz, 3H).

### Step C. Preparation of Int 44c

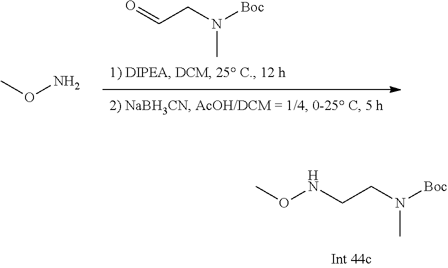

To a solution of O-methylhydroxylamine (1.45 g, 17.3 mmol, 1.32 mL, 1.50 eq) in dichloromethane (20.0 mL) were added diisopropylethylamine Int 44b (4.48 g, 34.6 mmol, 6.03 mL, 3.00 eq) and tert-butyl methyl(2-oxoethyl)carbamate (2.00 g, 11.6 mmol, 1.00 eq). The mixture was stirred at 25° C. for 12 h. Then the mixture was concentrated to afford a residue. The residue was dissolved in acetic acid (5.00 mL) and dichloromethane (20.0 mL), then sodium cyanoborohydride (1.09 g, 17.3 mmol, 1.50 eq) was added to the mixture at 0° C. The mixture was stirred at 25° C. for 5 h. The mixture was concentrated under reduced pressure to give a residue. The residue was purified by column chromatography (SiO₂, petroleum ether/ethyl acetate=1/0 to 2/1) to afford tert-butyl (2-(methoxyamino)ethyl)(methyl)carbamate Int 44c (1.40 g, 4.80 mmol, 41% yield, 70% purity) as yellow oil. ¹H NMR (400 MHz, DMSO-d₆) δ=6.77-6.20 (m, 1H), 3.38 (s, 3H), 3.24 (t, J=6.4 Hz, 2H), 2.85 (br t, J=6.4 Hz, 2H), 2.78 (br s, 3H), 1.38 (s, 9H).

### Step D. Preparation of Int 44d

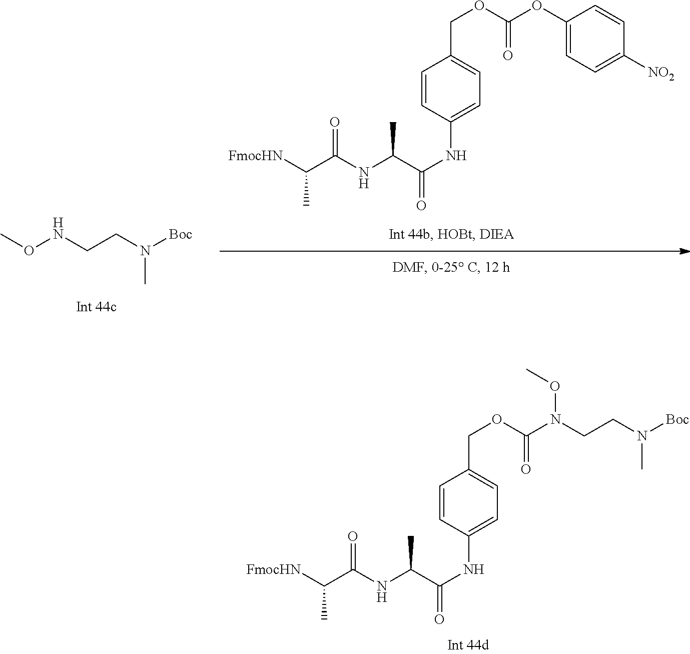

To a solution of tert-butyl (2-(methoxyamino)ethyl)(methyl)carbamate Int 44c (400 mg, 1.37 mmol, 1.00 eq) and (9H-fluoren-9-yl)methyl ((5)-1-(((5)-1-((4-((((4-nitrophenoxy)carbonyl)oxy)methyl)phenyl)amino)-1-oxopropan-2-yl)amino)-1-oxopropan-2-yl)carbamate Int 44b (895 mg, 1.37 mmol, 1.00 eq) in N,N-dimethylformamide (10.0 mL) were added 1-hydroxybenzotriazole (370 mg, 2.74 mmol, 2.00 eq) and diisopropylethylamine (531 mg, 4.11 mmol, 716 μL, 3.00 eq) at 0° C. The mixture was stirred at 25° C. for 12 h. The mixture was concentrated under reduced pressure to give a residue. The residue was purified by reversed-phase column (C18, 120 g; condition: water/acetonitrile=1/0-0/1, 0.1% formic acid) and lyophilized to afford 4-((S)-2-((S)-2-((((9H-fluoren-9-yl)methoxy)carbonyl)amino)propanamido) propanamido)benzyl (2-((tert-butoxycarbonyl)(methyl)amino)ethyl)(methoxy)carbamate Int 44d (593 mg, 496 μmol, 36% yield, 60% purity) as a white solid. MS (ESI) m/z 740.4 [M+Na]v

### Step E. Preparation of Int 44e

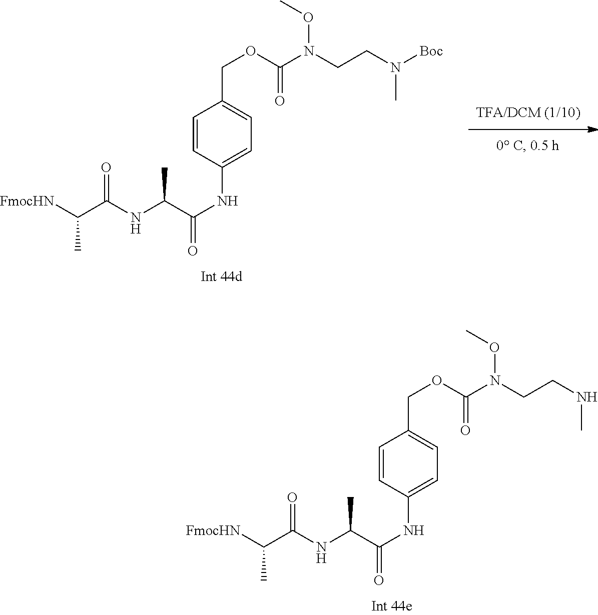

To a solution of 4-((S)-2-((S)-2-((((9H-fluoren-9-yl)methoxy)carbonyl)amino)propanamido)propanamido) benzyl (2-((tert-butoxycarbonyl)(methyl)amino)ethyl)(methoxy)carbamate Int 44d (593 mg, 496 μmol, 1.00 eq) in dichloromethane (10.0 mL) was added trifluoroacetic acid (1.54 g, 13.5 mmol, 1.00 mL, 27.2 eq) at 0° C. The mixture was stirred at 0° C. for 0.5 h. The mixture was concentrated under reduced pressure to give a residue. The residue was purified by reversed-phase column (C18, 80 g; condition: water/acetonitrile=1/0-0/1, 0.1% formic acid) and lyophilized to afford 4-((S)-2-((S)-2-((((9H-fluoren-9-yl)methoxy)carbonyl)amino)propanamido)propanamido)benzyl methoxy(2-(methylamino)ethyl)carbamate Int 44e (120 mg, 192 gmol, 39% yield, 99% purity) as a yellow solid. ¹H NMR (400 MHz, DMSO-d₆) δ=10.07-9.98 (m, 1H), 8.31 (br s, 1H), 8.16 (br d, J=6.8 Hz, 1H), 7.89 (d, J=7.6 Hz, 2H), 7.72 (br t, J=7.6 Hz, 2H), 7.60 (br d, J=8.4 Hz, 2H), 7.44-7.38 (m, 2H), 7.36-7.29 (m, 4H), 5.06 (s, 2H), 4.40 (br t, J=7.2 Hz, 1H), 4.29-4.19 (m, 3H), 4.12-4.06 (m, 1H), 3.60 (s, 3H), 3.57 (br t, J=6.4 Hz, 2H), 2.74 (br t, J=6.4 Hz, 2H), 2.32 (s, 3H), 1.31 (d, J=7.2 Hz, 3H), 1.23 (br d, J=7.2 Hz, 3H). MS (ESI) m/z 618.3 [M+H]⁺

### Step F. Preparation of Int 44f

To a solution of 1-((2-(2,6-dioxopiperidin-3-yl)-4-hydroxy-1-oxoisoindolin-5-yl)methyl)-3-(3-isopropyl-4-methylphenyl)urea IG-50 (100 mg, 215 μmol, 1.00 eq) in N,N-dimethylformamide (2.00 mL) was added triethylamine (174 mg, 1.72 mmol, 239 μL, 8.00 eq) and 4-nitrophenyl carbonochloridate (65.1 mg, 323 μmol, 1.50 eq). The mixture was stirred at 0° C. for 0.5 h. The mixture was concentrated under reduced pressure to give a residue. The residue was purified by reversed-phase column (C18, 80 g; condition: water/acetonitrile=1/0-0/1, 0.1% formic acid) and lyophilized to afford 2-(2,6-dioxopiperidin-3-yl)-5-((3-(3-isopropyl-4-methylphenyl)ureido)methyl)-1-oxoisoindolin-4-yl (4-nitrophenyl) carbonate Int 44f (100 mg, 79.4 μmol, 37% yield, 50% purity) as a white solid. MS (ESI) m/z 630.2 [M+H]⁺

### Step G. Preparation of Int 44g

To a solution of 2-(2,6-dioxopiperidin-3-yl)-5-((3-(3-isopropyl-4-methylphenyl)ureido)methyl)-1-oxoisoindolin-4-yl (4-nitrophenyl) carbonate Int 44f (80.0 mg, 63.5 μmol, 1.00 eq) and 4-((S)-2-((S)-2-((((9H-fluoren-9-yl)methoxy)carbonyl)amino)propanamido)propanamido)benzyl methoxy(2-(methylamino) ethyl)carbamate Int 44e (39.2 mg, 63.5 μmol, 1.00 eq) in NN-dimethylformamide (1.00 mL) was added diisopropylethylamine (16.4 mg, 127 μmol, 22.1 μL, 2.00 eq). The mixture was stirred at 0° C. for 0.5 h. The mixture was concentrated under reduced pressure to give a residue. The residue was purified by Prep-HPLC (column: Phenomenex Luna C18 150*25 mm*10 um; mobile phase: [water(formic acid)-acetonitrile];gradient: 50%-80% B over 10 min) and lyophilized to afford 4-((S)-2-((S)-2-((((9H-fluoren-9-yl)methoxy)carbonyl)amino)propanamido)propanamido)benzyl (2-((((2-(2,6-dioxopiperidin-3-yl)-5-((3-(3-isopropyl-4-methylphenyl)ureido)methyl)-1-oxoisoindolin-4-yl)oxy)carbonyl)(methyl)amino)ethyl) (methoxy)carbamate Int 44g (15.0 mg, 13.4 μmol, 21% yield, 99% purity) as a yellow solid. MS (ESI) m/z 1108.3 [M+H]⁺

### Step H. Preparation Int 44h

To a solution of 4-((S)-2-((S)-2-((((9H-fluoren-9-yl)methoxy)carbonyl)amino)propanamido)propanamido) benzyl(2-((((2-(2,6-dioxopiperidin-3-yl)-5-((3-(3-isopropyl-4-methylphenyl)ureido)methyl)-1-oxoisoindolin-4-yl)oxy)carbonyl)(methyl)amino)ethyl)(methoxy)carbamate Int 44g (15.0 mg, 13.5 μmol, 1.00 eq) in N,N-dimethylformamide (1.00 mL) was added piperidine (259 mg, 3.04 mmol, 0.300 mL, 224 eq). The mixture was stirred at 25° C. for 0.5 h. The mixture was adjusted with formic acid (50%) to pH=7 and concentrated to give a residue. The residue was purified by prep-HPLC (column: Waters xbridge 150*25 mm 10 um; mobile phase: [water (ammonium bicarbonate)-acetonitrile]; gradient: 37%-57% B over 8 min) and lyophilized to afford 4-((S)-2-((S)-2-((((9H-fluoren-9-yl)methoxy)carbonyl)amino)propanamido) propanamido)benzyl (2-((((2-(2,6-dioxopiperidin-3-yl)-5-((3-(3-isopropyl-4-methylphenyl)ureido)methyl)-1-oxoisoindolin-4-yl)oxy)carbonyl)(methyl)amino)ethyl)(methoxy)carbamate Int 44h (5.00 mg, 5.59 μmol, 41% yield, 99% purity) as a white solid. MS (ESI) m/z 886.4 [M+H]⁺

### Step I. Preparation of Compound IG-L-44

To a solution of 4-((S)-2-((S)-2-((((9H-fluoren-9-yl)methoxy)carbonyl)amino)propanamido)propanamido) benzyl (2-((((2-(2,6-dioxopiperidin-3-yl)-5-((3-(3-isopropyl-4-methylphenyl)ureido)methyl)-1-oxoisoindolin-4-yl)oxy)carbonyl)(methyl)amino)ethyl)(methoxy)carbamate Int 44h (5.00 mg, 5.64 μmol, 1.00 eq) and 34-(3-(2,5-dioxo-2,5-dihydro-1H-pyrrol-1-yl)-2-fluoro-6-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid (622 mg, 5.08 μmol, 0.900 eq) in N,N-dimethylformamide (0.500 mL) was added diisopropylethylamine (2.19 mg, 16.9 μmol, 2.95 μL, 3.00 eq). The mixture was stirred at 0° C. for 1 h. The mixture was concentrated under reduced pressure to give a residue. The residue was purified by prep-HPLC (column: Phenomenex luna C18 150*25 mm*10 um; mobile phase: [water (formic acid)-acetonitrile]; gradient: 25%-55% B over 20 min) and lyophilized to afford 34-(3-(2,5-dioxo-2,5-dihydro-1H-pyrrol-1-yl)-6-(3-(((2S)-1-(((2S)-1-((4-((((2-((((2-(2,6-dioxopiperidin-3-yl)-5-((3-(3-isopropyl-4-methylphenyl)ureido)methyl)-1-oxoisoindolin-4-yl)oxy)carbonyl)(methyl)amino)ethyl)(methoxy)carbamoyl)oxy)methyl)phenyl)amino)-1-oxopropan-2-yl)amino)-1-oxopropan-2-yl)amino)-3-oxopropyl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, compound IG-L-44 (1.29 mg, 0.61 mol, 11% yield, 92% purity) as a white solid. ¹H NMR (400 MHz, DMSO-d₆) δ=10.99 (br s, 1H), 10.08 (br s, 1H), 9.13 (br s, 1H), 8.49-8.08 (m, 3H), 7.63-7.48 (m, 4H), 7.29 (br d, J=7.6 Hz, 2H), 7.24 (s, 3H), 7.17-7.09 (m, 2H), 7.06-6.99 (m, 1H), 6.93 (br d, J=8.8 Hz, 1H), 5.14-5.01 (m, 3H), 4.39-3.88 (m, 30H), 3.69-3.58 (m, 6H), 3.17 (d, J=5.2 Hz, 4H), 3.06 (br s, 2H), 2.98-2.70 (m, 36H), 2.18 (s, 3H), 1.99-1.89 (m, 3H), 1.30 (br d, J=5.4 Hz, 3H), 1.19 (br d, J=7.2 Hz, 3H), 1.12 (br d, J=6.4 Hz, 6H). MS (ESI) m/z 972.8 [M/2+H]⁺

### Example L-45: Synthesis of 34-(6-(3-(((2S)-1-(((2S)-1-((4-((((2-(4-(3-((2-(1-(((((4-(((2S,3R,4S,5S,6S)-6-carboxy-3,4,5-trihydroxytetrahydro-2H-pyran-2-yl)oxy)benzyl)oxy)carbonyl)amino)methyl)-2,6-dioxopiperidin-3-yl)-1-oxoisoindolin-5-yl)methyl)ureido)-2-chlorophenethoxy)ethyl)(methyl)carbamoyl)oxy)methyl)phenyl)amino)-1-oxopropan-2-yl)amino)-1-oxopropan-2-yl)amino)-3-oxopropyl)-3-(2,5-dioxo-2,5-dihydro-1H-pyrrol-1-yl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, IG-L-45

[chemical structure]

### Step A. Preparation of Int 45a

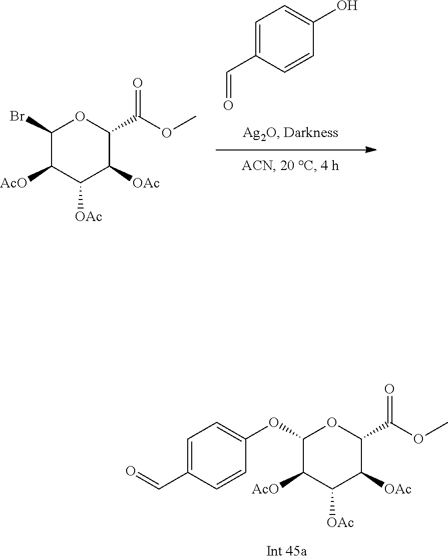

To a solution of (2R,3R,4S,5S,6S)-2-bromo-6-(methoxycarbonyl)tetrahydro-2H-pyran-3,4,5-triyl triacetate (30.0 g, 75.5 mmol, 1.00 eq) and 4-hydroxybenzaldehyde (10.2 g, 83.1 mmol, 1.10 eq) in acetonitrile (300 mL) was added silver oxide (26.3 g, 113 mmol, 1.50 eq), the mixture was stirred at 20° C. for 4 h under darkness. The mixture was concentrated to give a residue. The residue was purified by column chromatography (SiO₂, Petroleum ether/Ethyl acetate=5/1 to 1/1) and concentrated to afford (2S,3R,4S,5S,6S)-2-(4-formylphenoxy)-6-(methoxycarbonyl)tetrahydro-2H-pyran-3,4,5-triyl triacetate Int 45a (26.0 g, 59.3 mmol, 79% yield) as a yellow solid. ¹H NMR (400 MHz, DMSO-d₆) δ=9.92 (s, 1H), 7.92 (d, J=8.8 Hz, 2H), 7.20 (d, J=8.8 Hz, 2H), 5.86 (d, J=7.6 Hz, 1H), 5.52-5.46 (m, 1H), 5.21-5.04 (m, 2H), 4.77 (d, J=9.6 Hz, 1H), 3.64 (s, 3H), 2.04-2.00 (m, 9H).

### Step B. Preparation of Int 45b

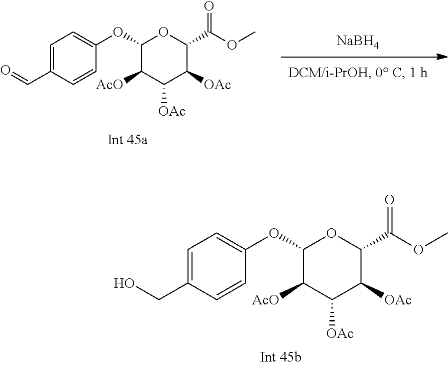 [SMILES: COC(=O)[C@H]1O[C@@H](Oc2ccc(C=O)cc2)[C@H](OC(C)=O)[C@@H](OC(C)=O)[C@@H]1OC(C)=O ; "NaBH4" ; COC(=O)[C@H]1O[C@@H](Oc2ccc(CO)cc2)[C@H](OC(C)=O)[C@@H](OC(C)=O)[C@@H]1OC(C)=O]

To a solution of (2S,3R,4S,5S,6S)-2-(4-formylphenoxy)-6-(methoxycarbonyl)tetrahydro-2H-pyran-3,4,5-triyl triacetate Int 45a (10.0 g, 22.8 mmol, 1.00 eq) in dichloromethane (100 mL) and isopropanol (20.0 mL) was added sodium borohydride (2.15 g, 56.8 mmol, 2.49 eq) at 0° C., the mixture was stirred at 0° C. for 1 h. The mixture was quenched with water (200 mL) and extracted with dichloromethane (100 mL×3). The organic phase was collected and concentrated to afford (2S,3R,4S,5S,6S)-2-(4-(hydroxymethyl)phenoxy)-6-(methoxycarbonyl)tetrahydro-2H-pyran-3,4,5-triyl triacetate Int 45b (10.0 g, crude) as yellow oil. ¹H NMR (400 MHz, DMSO-d₆) δ=7.27 (d, J=8.8 Hz, 2H), 6.95 (d, J=8.8 Hz, 2H), 5.62 (d, J=7.6 Hz, 1H), 5.47 (t, J=9.6 Hz, 1H), 5.16-5.03 (m, 3H), 4.70 (d, J=9.6 Hz, 1H), 4.43 (s, 2H), 3.64 (s, 3H), 2.03-1.99 (m, 9H).

### Step C. Preparation of Int 45c

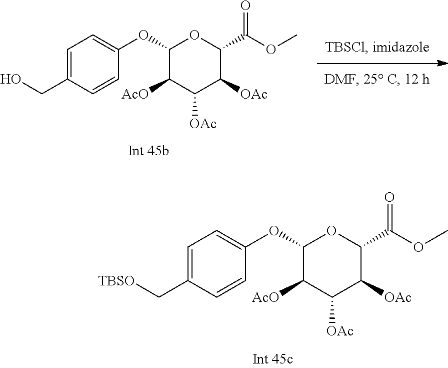 [SMILES: COC(=O)[C@H]1O[C@@H](Oc2ccc(CO)cc2)[C@H](OC(C)=O)[C@@H](OC(C)=O)[C@@H]1OC(C)=O ; "TBSCl, imidazole" ; COC(=O)[C@H]1O[C@@H](Oc2ccc(CO[Si](C)(C)C(C)(C)C)cc2)[C@H](OC(C)=O)[C@@H](OC(C)=O)[C@@H]1OC(C)=O]

To a solution of (2S,3R,4S,5S,6S)-2-(4-(hydroxymethyl)phenoxy)-6-(methoxycarbonyl)tetrahydro-2H-pyran-3,4,5-triyl triacetate Int 45b (10.0 g, 22.7 mmol, 1.00 eq) in dimethylformamide (100 mL) was added tert-butyl-chloro-dimethylsilane (5.13 g, 34.1 mmol, 4.19 mL, 1.50 eq) and imidazole (3.09 g, 45.4 mmol, 2.00 eq), the mixture was stirred at 25° C. for 12 h. The reaction mixture was diluted with water (300 mL) and exacted with ethyl acetate (3×100 mL). The organic phase was separated, washed with brine (2×100 mL), dried over sodium sulfate, filtered and concentrated under reduced pressure to give a residue. The residue was purified by column chromatography (SiO₂, petroleum ether/ethyl acetate=5/1 to 1/1) and concentrated to afford (2S,3R,4S,5S,6S)-2-(4-(((tert-butyldimethylsilyl)oxy)methyl)phenoxy)-6-(methoxycarbonyl)tetrahydro-2H-pyran-3,4,5-triyl triacetate Int 45c (9.00 g, 16.2 mmol, 71% yield) as yellow oil. ¹H NMR (400 MHz, DMSO-d₆) δ=7.26 (d, J=8.8 Hz, 2H), 6.96 (d, J=8.8 Hz, 2H), 5.62 (d, J=8.0 Hz, 1H), 5.46 (t, J=9.6 Hz, 1H), 5.11-5.01 (m, 2H), 4.69 (d, J=10.0 Hz, 1H), 4.64 (s, 2H), 3.63 (s, 3H), 2.02-1.98 (m, 9H), 0.89 (s, 9H), 0.06 (s, 6H).

### Step D. Preparation of Int 45d

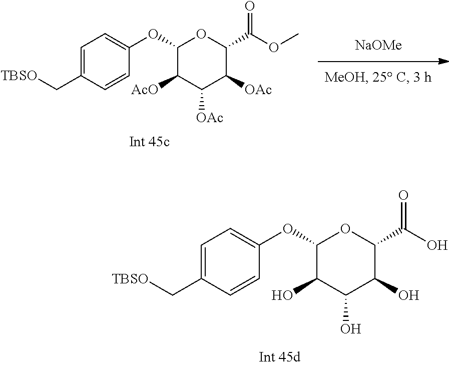 [SMILES: COC(=O)[C@H]1O[C@@H](Oc2ccc(CO[Si](C)(C)C(C)(C)C)cc2)[C@H](OC(C)=O)[C@@H](OC(C)=O)[C@@H]1OC(C)=O ; CC(C)(C)[Si](C)(C)OCc1ccc(O[C@@H]2O[C@H](C(=O)O)[C@@H](O)[C@H](O)[C@H]2O)cc1 ; "NaOMe"]

To a solution of (2S,3R,4S,5S,6S)-2-(4-(((tert-butyldimethylsilyl)oxy)methyl)phenoxy)-6-(methoxycarbonyl)tetrahydro-2H-pyran-3,4,5-triyl triacetate Int 45c (10.0 g, 18.0 mmol, 1.00 eq) in methanol (100 mL) was added sodium methanolate (1.95 g, 36.1 mmol, 2.00 eq), the mixture was stirred at 25° C. for 3 h. The mixture was filtered to give filtrate. The filtrate was purified by reversed-phase column (column: spherical C18, 20-45 um, 100 Å, SW 40, mobile phase: [water (0.1% ammonium hydrogen carbonate)-acetonitrile) and lyophilized to afford (2S,3S,4S,5R,6S)-6-(4-(((tert-butyldimethylsilyl)oxy)methyl)phenoxy)-3,4,5-trihydroxytetrahydro-2H-pyran-2-carboxylic acid Int 45d (6.00 g, 14.5 mmol, 80% yield) as yellow oil. MS (ESI) m/z 437.1 [M+Na]⁺

### Step E. Preparation of Int 45e

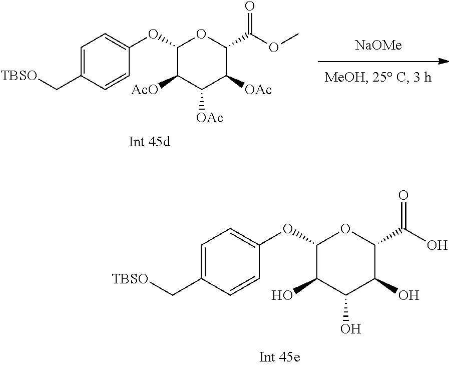 [SMILES: COC(=O)[C@H]1O[C@@H](Oc2ccc(CO[Si](C)(C)C(C)(C)C)cc2)[C@H](OC(C)=O)[C@@H](OC(C)=O)[C@@H]1OC(C)=O ; CC(C)(C)[Si](C)(C)OCc1ccc(O[C@@H]2O[C@H](C(=O)O)[C@@H](O)[C@H](O)[C@H]2O)cc1 ; "NaOMe"]

To a solution of (2S,3R,4S,5S,6S)-2-(4-(((tert-butyldimethylsilyl)oxy)methyl)phenoxy)-6-(methoxycarbonyl)tetrahydro-2H-pyran-3,4,5-triyl triacetate Int 45d (10.0 g, 18.0 mmol, 1.00 eq) in methanol (100 mL) was added sodium methanolate (1.95 g, 36.1 mmol, 2.00 eq), the mixture was stirred at 25° C. for 3 h. The mixture was filtered to give filtrate. The filtrate was purified by reversed-phase column (column: spherical C18, 20-45 um, 100 Å, SW 40, mobile phase: [water (0.1% ammonium hydrogen carbonate)-acetonitrile) and lyophilized to afford (2S,3S,4S,5R,6S)-6-(4-(((tert-butyldimethylsilyl)oxy)methyl)phenoxy)-3,4,5-trihydroxytetrahydro-2H-pyran-2-carboxylic acid Int 45e (6.00 g, 14.5 mmol, 80% yield) as yellow oil. MS (ESI) m/z 437.1 [M+Na]f

### Step F. Preparation of Int 45f

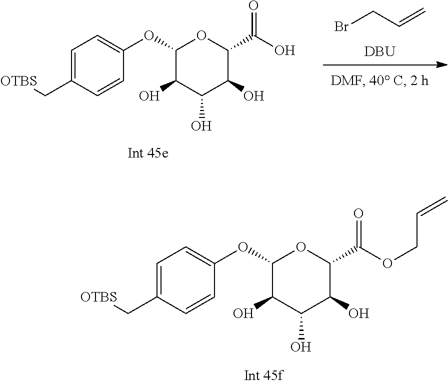

To a solution of (2S,3S,4S,5R,6S)-6-(4-(((tert-butyldimethylsilyl)oxy)methyl)phenoxy)-3,4,5-trihydroxytetrahydro-2H-pyran-2-carboxylic acid Int 45e (6.00 g, 14.5 mmol, 1.00 eq) in dimethylformamide (30.0 mL) was added 2,3,4,6,7,8,9,10-octahydropyrimido[1,2-a]azepine (4.41 g, 29.0 mmol, 4.36 mL, 2.00 eq) and 3-bromoprop-1-ene (5.25 g, 43.4 mmol, 3.00 eq), the mixture was stirred at 40° C. for 2 h under nitrogen atmosphere. The mixture was filtered to give a filtrate. The filtrate was purified by reversed-phase column (column: spherical C18, 20-45 um, 100 Å, SW 40, mobile phase: [water (0.10% ammonium acid carbonate))-acetonitrile) and lyophilized to afford (E)-prop-1-en-1-yl (2S,3S,4S,5R,6S)-6-(4-(((tert-butyldimethylsilyl)oxy) methyl) phenoxy)-3,4,5-trihydroxytetrahydro-2H-pyran-2-carboxylate Int 45f (4.00 g, 8.80 mmol, 61% yield) as yellow oil. ¹H NMR (400 MHz, DMSO-d₆) δ=7.16 (d, J=8.8 Hz, 2H), 6.92 (d, J=8.8 Hz, 2H), 5.84 (tdd, J=5.2, 10.4, 17.2 Hz, 1H), 5.42 (d, J=4.8 Hz, 1H), 5.38 (d, J=5.6 Hz, 1H), 5.29-5.23 (m, 1H), 5.21 (d, J=5.2 Hz, 1H), 5.13 (dd, J=1.6, 10.4 Hz, 1H), 5.01 (d, J=7.6 Hz, 1H), 4.64-4.50 (m, 4H), 4.01 (d, J=9.6 Hz, 1H), 3.36 (br d, J=5.6 Hz, 1H), 3.22 (br dd, J=5.2, 7.6 Hz, 2H), 0.83 (s, 9H), 0.00 (s, 6H).

### Step G. Preparation of Int 45g

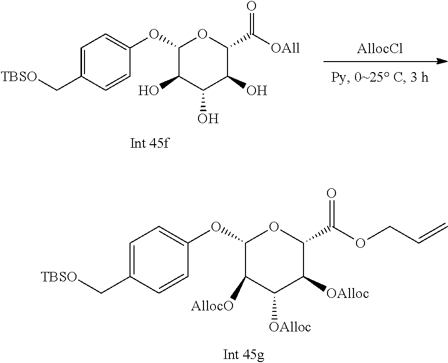

To a solution of (E)-prop-1-en-1-yl (2S,3S,4S,5R,6S)-6-(4-(((tert-butyldimethylsilyl)oxy)methyl)phenoxy)-3,4,5-trihydroxytetrahydro-2H-pyran-2-carboxylate Int 45f (4.00 g, 8.80 mmol, 1.00 eq) in pyridine (50.0 mL) was added allyl carbonochloridate (31.8 g, 264 mmol, 28.0 mL, 30.0 eq) at 0° C., the mixture was stirred at 25° C. for 3 h under nitrogen atmosphere. The reaction mixture was concentrated under reduced pressure to give a residue. The residue was purified by reversed-phase column (column: spherical C18, 20-45 um, 100 Å, SW 40, mobile phase: [water (0.1% ammonium hydrogen carbonate)-acetonitrile) and lyophilized to afford (E)-prop-1-en-1-yl (2S,3S,4S,5R,6S)-3,4,5-tris(((allyloxy)carbonyl)oxy)-6-(4-(((tert-butyldimethylsilyl) oxy)methyl)phenoxy)tetrahydro-2H-pyran-2-carboxylate Int 45g (3.50 g, 4.95 mmol, 56% yield) as yellow oil. ¹H NMR (400 MHz, DMSO-d₆) δ=7.21 (d, J=8.8 Hz, 2H), 6.90 (d, J=8.8 Hz, 2H), 5.89-5.76 (m, 4H), 5.61 (d, J=8.0 Hz, 1H), 5.38-5.32 (m, 1H), 5.28-5.25 (m, 1H), 5.22 (tdd, J=1.6, 3.2, 5.2 Hz, 3H), 5.19-5.17 (m, 3H), 5.16 (dd, J=12, 2.0 Hz, 1H), 5.07-4.99 (m, 2H), 4.74 (d, J=10.0 Hz, 1H), 4.61-4.44 (m, 10H), 0.83 (s, 9H), 0.00 (s, 6H). MS (ESI) m/z 724.3 [M+H₂O]⁺

### Step H. Preparation of Int 45h

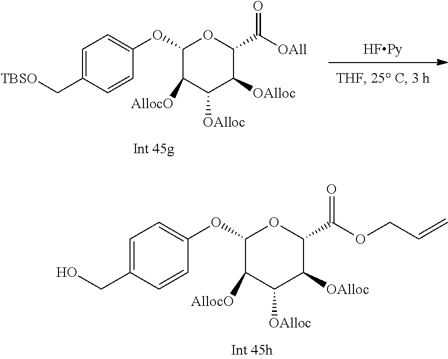

To a solution of (E)-prop-1-en-1-yl (2S,3S,4S,5R,6S)-3,4,5-tris(((allyloxy)carbonyl)oxy)-6-(4-(((tert-butyldimethylsilyl)oxy)methyl)phenoxy)tetrahydro-2H-pyran-2-carboxylate Int 45g (3.50 g, 4.95 mmol, 1.00 eq) in tetrahydrofuran (20.0 mL) was added pyridine;hydrofluoride (6.42 g, 45.3 mmol, 5.83 mL, 70% purity, 9.15 eq), the mixture was stirred at 25° C. for 3 h. The reaction mixture was concentrated under reduced pressure to give a residue. The residue was purified by reversed-phase column (column: spherical C18, 20-45 um, 100 Å, SW 40, mobile phase: [water (0.1% Formic Acid)-acetonitrile) and lyophilized to afford (E)-prop-1-en-1-yl (2S,3S,4S,5R,6S)-3,4,5-tris(((allyloxy)carbonyl)oxy)-6-(4-(hydroxymethyl)phenoxy)tetrahydro-2H-pyran-2-carboxylate Int 45h (2.30 g, 3.88 mmol, 78% yield) as yellow oil. ¹H NMR (400 MHz, DMSO-d₆) δ=7.28 (d, J=8.8 Hz, 2H), 6.95 (d, J=8.8 Hz, 2H), 5.97-5.82 (m, 4H), 5.67 (d, J=8.0 Hz, 1H), 5.46-5.39 (m, 1H), 5.36-5.32 (m, 1H), 5.30 (ddd, J=1.6, 3.2, 4.8 Hz, 2H), 5.28-5.21 (m, 5H), 5.18-5.04 (m, 3H), 4.81 (d, J=10.0 Hz, 1H), 4.71-4.58 (m, 7H), 4.56 (br d, J=5.6 Hz, 1H), 4.44 (d, J=5.6 Hz, 2H).

### Step I. Preparation of Int 45i

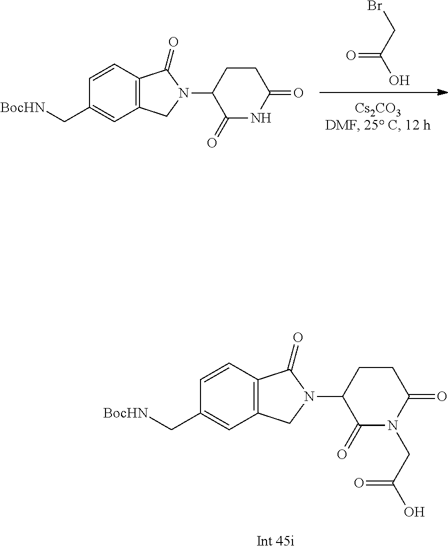 [SMILES: CC(C)(C)OC(=O)NCc1ccc2c(c1)CN(C1CCC(=O)NC1=O)C2=O ; O=C(O)CBr ; CC(C)(C)OC(=O)NCc1ccc2c(c1)CN(C1CCC(=O)N(CC(=O)O)C1=O)C2=O]

To a solution of tert-butyl ((2-(2,6-dioxopiperidin-3-yl)-1-oxoisoindolin-5-yl)methyl)carbamate (1.00 g, 2.68 mmol, 1.00 eq) and 2-bromoacetic acid (558 mg, 4.02 mmol, 289 μL, 1.50 eq) in dimethylformamide (10.0 mL) was added cesium carbonate (2.62 g, 8.03 mmol, 3.0 eq), the mixture was stirred at 25° C. for 12 h. The mixture was filtered to give filtrate. The filtrate was purified by reversed-phase column (column: spherical C18, 20-45 um, 100 Å, SW 40, mobile phase: [water (0.1% Formic Acid)-acetonitrile) and lyophilized to afford 2-(3-(5-(((tert-butoxycarbonyl)amino)methyl)-1-oxoisoindolin-2-yl)-2,6-dioxopiperidin-1-yl)acetic acid Int 45i (1.50 g, 3.48 mmol, 65% yield) as a yellow solid. ¹H NMR (400 MHz, DMSO-d₆) δ=13.25-12.68 (m, 1H), 7.70 (d, J=7.6 Hz, 1H), 7.51 (br t, J=5.6 Hz, 1H), 7.46 (s, 1H), 7.39 (br d, J=8.0 Hz, 1H), 5.27 (dd, J=5.2, 13.2 Hz, 1H), 4.54-4.46 (m, 1H), 4.44-4.18 (m, 5H), 3.19-3.04 (m, 1H), 2.98-2.80 (m, 1H), 2.44-2.31 (m, 1H), 2.15-2.06 (m, 1H), 1.40 (s, 9H). MS (ESI) m/z 432.2 [M+H]⁺

### Step J. Preparation of Int 45j

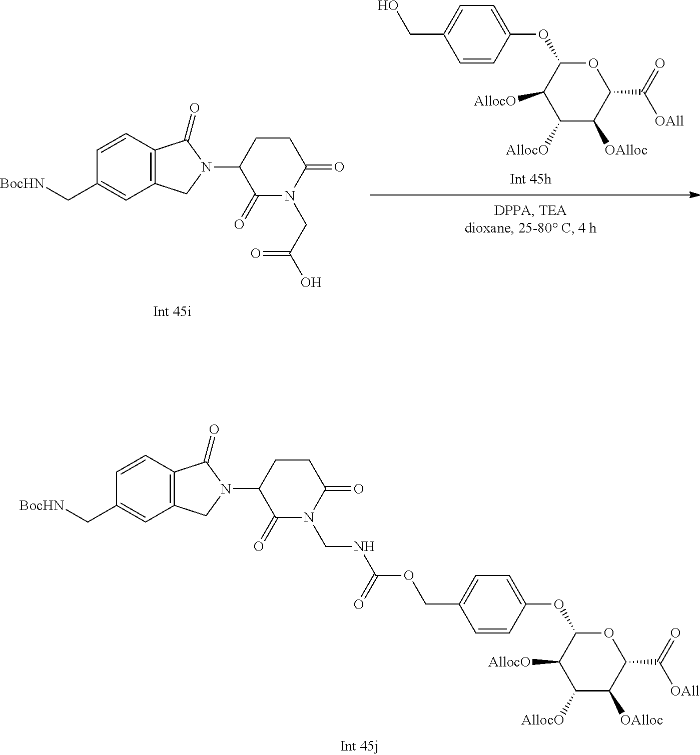

A mixture of 2-(3-(5-(((tert-butoxycarbonyl)amino)methyl)-1-oxoisoindolin-2-yl)-2,6-dioxopiperidin-1-yl)acetic acid Int 45i (500 mg, 1.16 mmol, 1.0 eq), [azido(phenoxy)phosphoryl]oxybenzene (478 mg, 1.74 mmol, 375 μL, 1.50 eq) and triethylamine (235 mg, 2.32 mmol, 323 μL, 2.00 eq) in dioxane (2.00 mL) was stirred at 25° C. for 1 h, then (E)-prop-1-en-1-yl (2S,3S,4S,5R,6S)-3,4,5-tris(((allyloxy)carbonyl)oxy)-6-(4-(hydroxymethyl) phenoxy)tetrahydro-2H-pyran-2-carboxylate Int 45h (687 mg, 1.16 mmol, 1.00 eq) was added, the mixture was stirred at 80° C. for 3 h. The reaction mixture was concentrated under reduced pressure to give a residue. The residue was purified by reversed-phase column (column: spherical C18, 20-45 um, 100 Å, SW 40, mobile phase: [water (0.1% Formic Acid)-acetonitrile) and lyophilized to afford (E)-prop-1-en-1-yl (2S,3S,4S,5R,6S)-3,4,5-tris(((allyloxy)carbonyl)oxy)-6-(4-(((((3-(5-(((tert-butoxycarbonyl)amino)methyl)-1-oxoisoindolin-2-yl)-2,6-dioxopiperidin-1-yl)methyl)carbamoyl)oxy)methyl)phenoxy)tetrahydro-2H-pyran-2-carboxylate Int 45j (600 mg, 588 μmol, 17% yield) as a white solid. (¹H NMR from the pilot). ¹H NMR (400 MHz, DMSO-d₆) δ=7.70 (d, J=7.6 Hz, 1H), 7.50 (br t, J=6.4 Hz, 1H), 7.45 (br s, 1H), 7.41-7.32 (m, 4H), 7.08 (br dd, J=5.6, 8.0 Hz, 1H), 6.99 (br dd, J=5.2, 8.4 Hz, 1H), 5.97-5.86 (m, 4H), 5.76-5.65 (m, 1H), 5.44-5.36 (m, 2H), 5.35-5.23 (m, 10H), 5.19-5.07 (m, 4H), 5.00-4.90 (m, 2H), 4.71-4.58 (m, 10H), 4.56-4.42 (m, 3H), 4.24 (br d, J=6.0 Hz, 2H), 1.40 (s, 9H). MS (ESI) m/z 1021.5 [M+H]⁺

### Step K. Preparation of Int 45k

A mixture of tert-butyl (2-(2-chloro-4-((phenoxycarbonyl)amino)phenethoxy)ethyl)(methyl)carbamate Int 1j (73.0 mg, 162 μmol, 1.00 eq), (E)-prop-1-en-1-yl (2S,3S,4S,5R,6S)-3,4,5-tris(((allyloxy)carbonyl)oxy)-6-(4-(((((3-(5-(aminomethyl)-1-oxoisoindolin-2-yl)-2,6-dioxopiperidin-1-yl)methyl)carbamoyl)oxy)methyl)phenoxy) tetrahydro-2H-pyran-2-carboxylate Int 45j (150 mg, 162 μmol, 1.00 eq) and triethylamine (49.5 mg, 488 μmol, 68.0 μL, 3.00 eq) in dimethyl formamide (2.00 mL) was stirred at 40° C. for 6 h. The reaction mixture was filtered to give a filtrate. The filtrate was purified by reversed phase (0.1% formic acid) and lyophilized afford (E)-prop-1-en-1-yl (2S,3S,4S,5R,6S)-3,4,5-tris(((allyloxy)carbonyl)oxy)-6-(4-(((((3-(5-((3-(4-(2-(2-((tert-butoxycarbonyl)(methyl)amino)ethoxy)ethyl)-3-chlorophenyl)ureido)methyl)-1-oxoisoindolin-2-yl)-2,6-dioxopiperidin-1-yl)methyl)carbamoyl)oxy)methyl)phenoxy)tetrahydro-2H-pyran-2-carboxylate Int 45k (160 mg, 125 μmol, 77% yield) as a white solid. MS (ESI) m/z 1175.3 [M-100+H]⁺

### Step L. Preparation of Int 45l

To a solution of (E)-prop-1-en-1-yl (2S,3S,4S,5R,6S)-3,4,5-tris(((allyloxy)carbonyl)oxy)-6-(4-(((((3-(5-((3-(4-(2-(2-((tert-butoxycarbonyl)(methyl)amino)ethoxy)ethyl)-3-chlorophenyl)ureido)methyl)-1-oxoisoindolin-2-yl)-2,6-dioxopiperidin-1-yl)methyl)carbamoyl)oxy)methyl)phenoxy)tetrahydro-2H-pyran-2-carboxylate Int 45k (160 mg, 125 μmol, 1.00 eq) in dichloromethane (1.50 mL) was added trifluoroacetic acid (160 L), the mixture was stirred at 25° C. for 0.5 h. The reaction mixture was concentrated to give a residue and lyophilized to afford (E)-prop-1-en-1-yl (2S,3S,4S,5R,6S)-3,4,5-tris(((allyloxy)carbonyl)oxy)-6-(4-(((((3-(5-((3-(3-chloro-4-(2-(2-(methylamino)ethoxy)ethyl)phenyl)ureido)methyl)-1-oxoisoindolin-2-yl)-2,6-dioxopiperidin-1-yl)methyl)carbamoyl)oxy)methyl)phenoxy)tetrahydro-2H-pyran-2-carboxylate Int 45l (140 mg, crude) as a white solid. MS (ESI) m/z 1175.6 [M+H]⁺

### Step M. Preparation of Int 45m

A mixture of (E)-prop-1-en-1-yl (2S,3S,4S,5R,6S)-3,4,5-tris(((allyloxy)carbonyl)oxy)-6-(4-(((((3-(5-((3-(3-chloro-4-(2-(2-(methylamino)ethoxy)ethyl)phenyl)ureido)methyl)-1-oxoisoindolin-2-yl)-2,6-dioxopiperidin-1-yl)methyl)carbamoyl)oxy)methyl)phenoxy)tetrahydro-2H-pyran-2-carboxylate Int 45l (60.0 mg, 51.0 μmol, 1.00 eq), (9H-fluoren-9-yl)methyl ((S)-1-(((S)-1-((4-((((4-nitrophenoxy)carbonyl)oxy)methyl)phenyl)amino)-1-oxopropan-2-yl)amino)-1-oxopropan-2-yl)carbamate Int 44b (36.6 mg, 56.2 μmol, 1.10 eq) and triethylamine (15.5 mg, 153 μmol, 21.3 μL, 3.00 eq) in dimethyl formamide (2.00 mL) was stirred at 25° C. for 2 h. The reaction mixture was filtered to give a filtrate. The filtrate was purified by prep-HPLC (column: Phenomenex luna C18 150*25 mm*10 um; mobile phase: [water(formic acid)-acetonitrile];gradient: 66%-86% B over 10 min) to afford (E)-prop-1-en-1-yl (2S,3S,4S,5R,6S)-6-(4-(((((3-(5-((3-(4-(2-(2-((((4-((S)-2-((S)-2-((((9H-fluoren-9-yl)methoxy)carbonyl)amino)propanamido)propanamido)benzyl)oxy)carbonyl)(methyl)amino)ethoxy)ethyl)-3-chlorophenyl)ureido)methyl)-1-oxoisoindolin-2-yl)-2,6-dioxopiperidin-1-yl)methyl)carbamoyl)oxy)methyl)phenoxy)-3,4,5-tris(((allyloxy)carbonyl)oxy)tetrahydro-2H-pyran-2-carboxylate Int 45m (50.0 mg, 29.6 μmol, 58% yield) as a white solid.

### Step N. Preparation of Int 45n

To a solution of (E)-prop-1-en-1-yl (2S,3S,4S,5R,6S)-6-(4-(((((3-(5-((3-(4-(2-(2-((((4-((S)-2-((S)-2-((((9H-fluoren-9-yl)methoxy)carbonyl)amino)propanamido)propanamido)benzyl)oxy)carbonyl)(methyl)amino) ethoxy)ethyl)-3-chlorophenyl)ureido)methyl)-1-oxoisoindolin-2-yl)-2,6-dioxopiperidin-1-yl)methyl) carbamoyl)oxy)methyl)phenoxy)-3,4,5-tris(((allyloxy)carbonyl)oxy)tetrahydro-2H-pyran-2-carboxylate Int 45m (25.0 mg, 14.8 μmol, 1.00 eq) in dimethyl formamide (2.00 mL) was added pyrrolidine (5.26 mg, 74.0 μmol, 6.18 μL, 5.00 eq) and tetrakis(triphenylphosphine)palladium(0) (1.71 mg, 1.48 μmol, 0.100 eq), the mixture was stirred at 25° C. for 1 h. The mixture was filtered to give a filtrate. The filtrate was purified by prep-HPLC (column: Phenomenex luna C18 150*25 mm*10 um; mobile phase: [water(formic acid)-acetonitrile];gradient: 11%-41% B over 10 min) and lyophilized to afford (2S,3S,4S,5R,6S)-6-(4-(((((3-(5-((3-(4-(2-(2-((((4-((S)-2-((S)-2-aminopropanamido)propanamido)benzyl)oxy)carbonyl)(methyl)amino)ethoxy) ethyl)-3-chlorophenyl)ureido)methyl)-1-oxoisoindolin-2-yl)-2,6-dioxopiperidin-1-yl)methyl)carbamoyl)oxy) methyl)phenoxy)-3,4,5-trihydroxytetrahydro-2H-pyran-2-carboxylic acid Int 45n (10.0 mg, 8.51 μmol, 57% yield) as a white solid.

### Step O. Preparation of Compound IG-L-45

To a solution of 34-(3-(2,5-dioxo-2,5-dihydro-1H-pyrrol-1-yl)-2-fluoro-6-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid Int 36k (10.4 mg, 8.51 μmol, 1.00 eq) and (2S,3S,4S,5R,6S)-6-(4-(((((3-(5-((3-(4-(2-(2-((((4-((S)-2-((S)-2-aminopropanamido)propanamido)benzyl) oxy)carbonyl)(methyl)amino)ethoxy)ethyl)-3-chlorophenyl)ureido)methyl)-1-oxoisoindolin-2-yl)-2,6-dioxopiperidin-1-yl)methyl)carbamoyl)oxy)methyl)phenoxy)-3,4,5-trihydroxytetrahydro-2H-pyran-2-carboxylic acid Int 45n (10.0 mg, 8.51 μmol, 1.00 eq) in dimethyl formamide (1.00 mL) was added triethylamine (2.58 mg, 25.5 μmol, 3.55 μL, 3.00 eq). The reaction was stirred at 25° C. for 2 h. The reaction was filtered. The filtrate was purified by prep-HPLC (column: Phenomenex luna C18 150*25 mm*10 um; mobile phase: [water (formic acid)-acetonitrile]; gradient: 18%-48% B over 20 min) and lyophilized to afford 34-(6-(3-(((2S)-1-(((2S)-1-((4-((((2-(4-(3-((2-(1-(((((4-(((2S,3R,4S,5S,6S)-6-carboxy-3,4,5-trihydroxytetrahydro-2H-pyran-2-yl)oxy)benzyl)oxy)carbonyl)amino)methyl)-2,6-dioxopiperidin-3-yl)-1-oxoisoindolin-5-yl)methyl)ureido)-2-chlorophenethoxy)ethyl)(methyl)carbamoyl)oxy)methyl)phenyl)amino)-1-oxopropan-2-yl)amino)-1-oxopropan-2-yl)amino)-3-oxopropyl)-3-(2,5-dioxo-2,5-dihydro-1H-pyrrol-1-yl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid Compound IG-L-45 (3.69 mg, 1.60 mol, 19% yield, 97% purity) as a white solid. ¹H NMR (400 MHz, DMSO-d₆) δ=9.99-9.93 (m, 1H), 8.23-8.08 (m, 2H), 7.72-7.66 (m, 2H), 7.61 (br d, J=8.0 Hz, 2H), 7.50-7.42 (m, 2H), 7.33-7.23 (m, 6H), 7.22-7.09 (m, 3H), 7.09-6.92 (m, 3H), 5.36-5.30 (m, 1H), 5.20 (td, J=3.6, 7.6 Hz, 1H), 5.13-5.08 (m, 1H), 5.02-4.78 (m, 6H), 4.53-4.26 (m, 10H), 4.14-3.92 (m, 12H), 4.25-3.83 (m, 4H), 3.68-3.62 (m, 2H), 3.58-3.45 (m, 8H), 3.17 (s, 3H), 3.07-2.64 (m, 40H), 2.28-2.22 (m, 1H), 2.05-1.88 (m, 4H), 1.30 (br d, J=7.2 Hz, 3H), 1.21-1.15 (m, 3H). MS (ESI) m/z 1117.4 [M/2+2H]⁺

The compounds in Table 19 were prepared in a manner similar to that described for Compound IG-L-45 using the appropriate compound as starting material.

TABLE 19

| IG-L No. | Structure | ¹H NMR/MS |
|---|---|---|
| IG-L-62 | 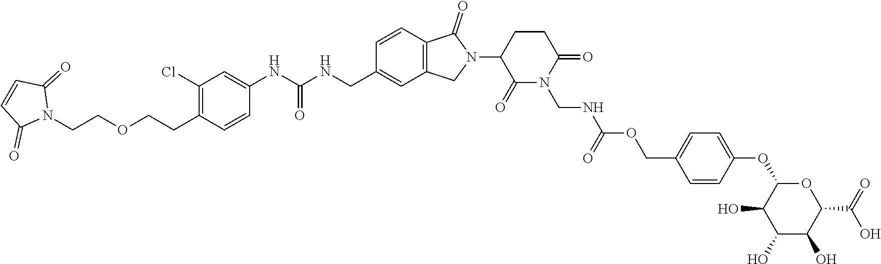 [SMILES: O=C(NCc1ccc2c(c1)CN(C1CCC(=O)N(CNC(=O)OCc3ccc(O[C@@H]4O[C@H](C(=O)O)[C@@H](O)[C@H](O)[C@H]4O)cc3)C1=O)C2=O)Nc1ccc(CCOCCN2C(=O)C=CC2=O)c(Cl)c1] | ¹H NMR (400 MHz, DMSO-d₆) δ = 9.73-9.60 (m, 1H), 7.99- 7.82 (m, 1H), 7.70-7.63 (m, 2H), 7.52 (s, 1H), 7.46 (t, J = 8.8 Hz, 1H), 7.30-7.24 (m, 2H), 7.24- 7.17 (m, 2H), 7.11-7.02 (m, 3H), 6.94 (d, J = 1.6 Hz, 2H), 5.18- 5.08 (m, 2H), 5.01-4.91 (m, 4H), 4.87-4.80 (m, 1H), 4.64 (br d, J = 2.8 Hz, 1H), 4.50-4.30 (m, 3H), 4.24-4.13 (m, 1H), 3.61- 3.50 (m, 8H), 3.35-3.28 (m, 3H), 2.96 (br d, J = 2.8 Hz, 1H), 2.81- 2.76 (m, 3H), 2.58 (br s, 1H), 2.28-2.18 (m, 1H), 2.05 (td, J = 2.4, 5.2 Hz, 1H). MS (ESI) m/z 971.4 [M + Na]⁺ |
| IG-L-81 | 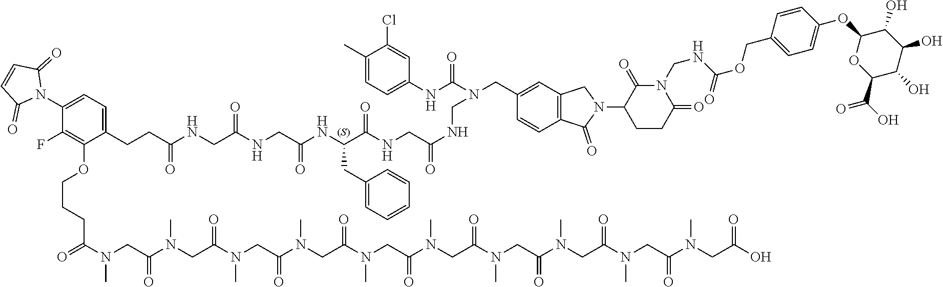 | ¹H NMR (400 MHz, DMSO-d₆) δ = 9.67 (br s, 1H), 9.04 (br s, 1H), 8.48 (br s, 1H), 8.21 (br s, 2H), 8.08 (br s, 1H), 7.72 (d, J = 8.0 Hz, 1H), 7.62 (d, J = 2.0 Hz, 1H), 7.59-7.50 (m, 2H), 7.46 (br d, J = 8.0 Hz, 1H), 7.35- 6.93 (m, 15H), 5.42 (br d, J = 2.4 Hz, 1H), 5.26-5.13 (m, 2H), 5.02 (br d, J = 7.2 Hz, 2H), 4.97-4.89 (m, 3H), 4.71-4.61 (m, 4H), 4.48-3.94 (m, 27H), 3.84-3.59 (m, 8H), 3.09-2.69 (m, 41H), 2.23-2.23 (m, 1H), 2.23 (s, 3H), 2.02-1.88 (m, 3H). LCMS (ESI) m/z 1101.9 [M/2 + 2H]⁺ |

### Example L-52: Synthesis of 34-(6-(3-(((2S)-1-(((2S)-1-((4-(((5-((3-(3-chloro-4-methylphenyl)ureido)methyl)-2-(2,6-dioxopiperidin-3-yl)-1-oxoisoindolin-4-yl)oxy)methyl)-3-methoxyphenyl)amino)-1-oxopropan-2-yl)amino)-1-oxopropan-2-yl)amino)-3-oxopropyl)-3-(2,5-dioxo-2,5-dihydro-1H-pyrrol-1-yl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, IG-L-52

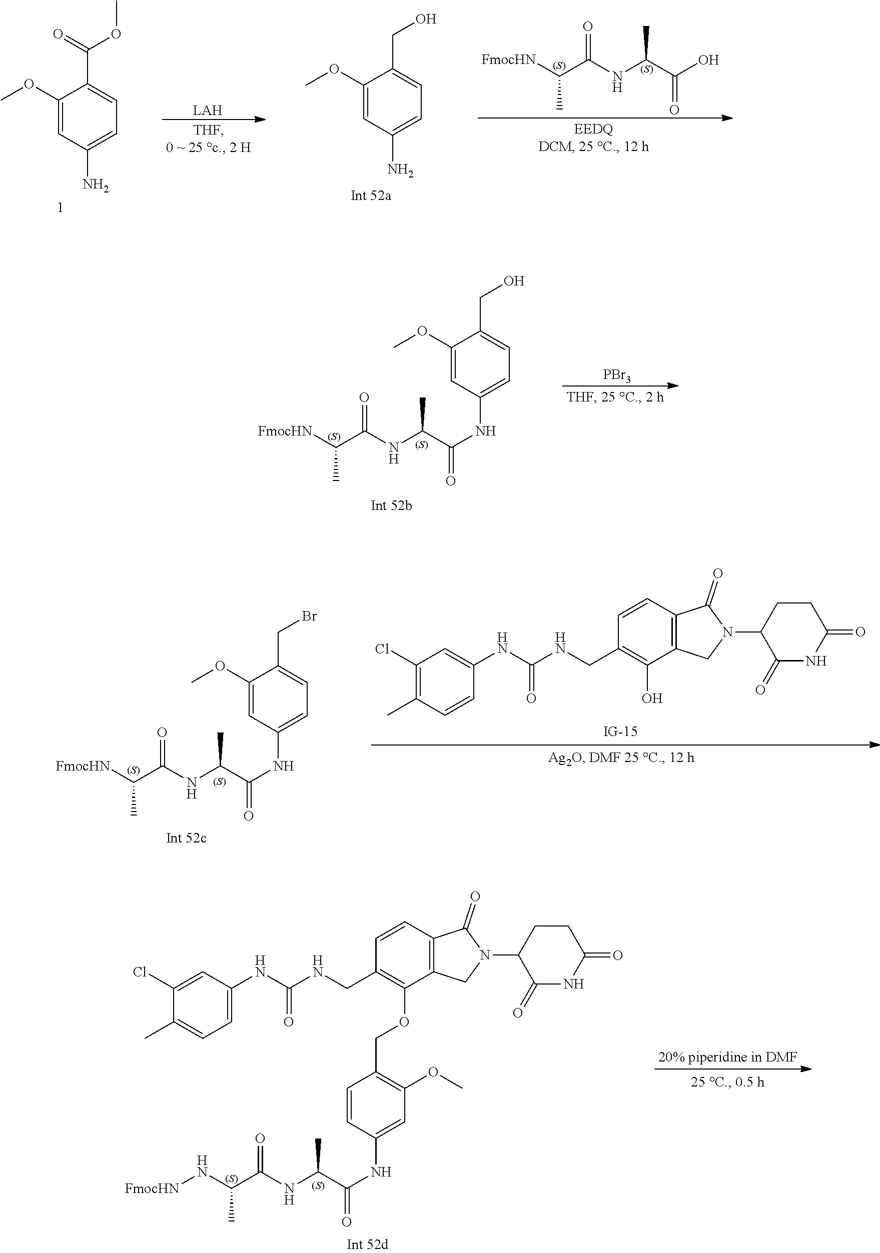

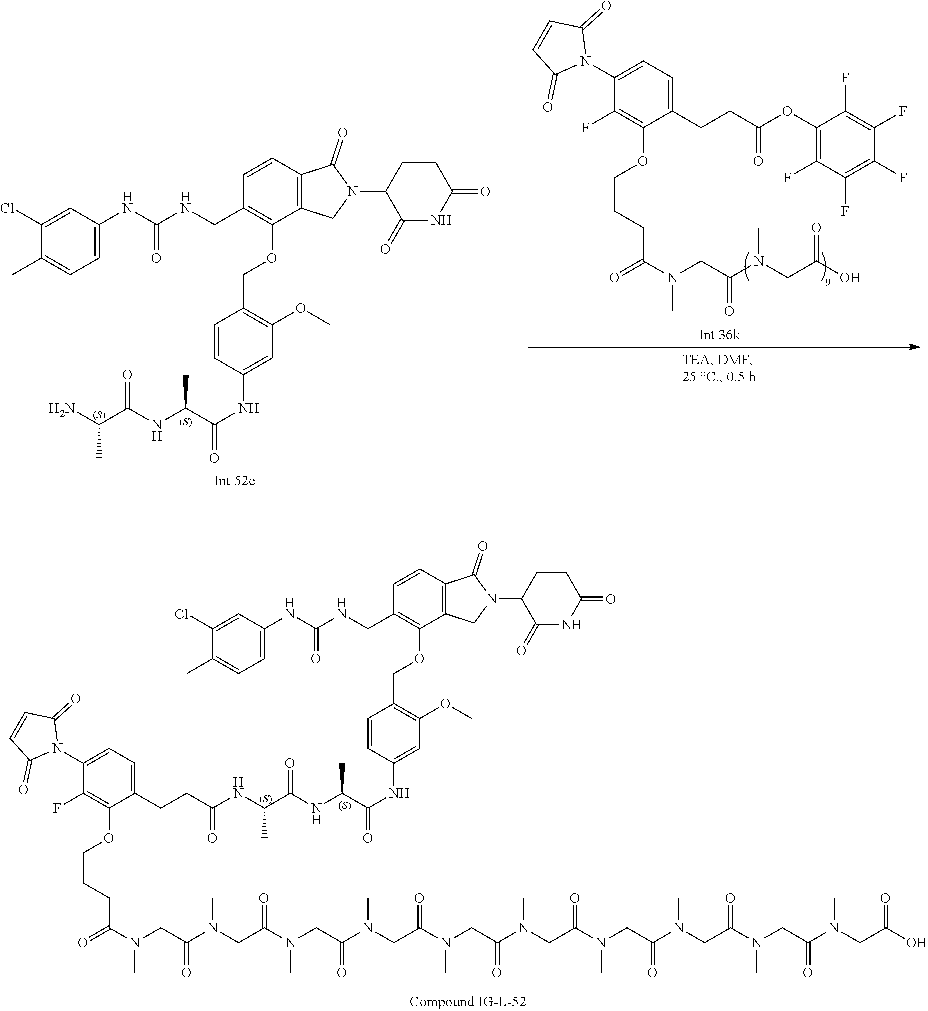

### Step A. Preparation of 52a

To a solution of methyl 4-amino-2-methoxybenzoate (10.0 g, 55.2 mmol, 1.00 eq) in tetrahydrofuran (200 mL) was added lithium aluminum hydride (4.19 g, 110 mmol, 2.00 eq) at 0° C. Then the mixture was stirred at 25° C. for 2 h. The mixture was quenched with water (4.19 mL), sodium hydroxide (15%, 4.19 mL) and water (8.40 mL) at 0° C. Then the mixture was dried over sodium sulfate and filtered. The filtrate was concentrated to give (4-amino-2-methoxy-phenyl)methanol Int 52a (8.00 g, crude) as yellow oil. ¹H NMR (400 MHz, DMSO-d₆) δ=6.92 (d, J=8.0 Hz, 1H), 6.18 (d, J=2.0 Hz, 1H), 6.10 (dd, J=2.0, 8.0 Hz, 1H), 4.95 (s, 2H), 4.54-4.45 (m, 1H), 4.29 (d, J=5.6 Hz, 2H), 3.66 (s, 3H).

### Step B. Preparation of 52b

To a solution of (4-amino-2-methoxyphenyl)methanol (8.00 g, 52.2 mmol, 1.00 eq) and (((9H-fluoren-9-yl)methoxy)carbonyl)-L-alanyl-L-alanine Int 52a (20.0 g, 52.2 mmol, 1.00 eq) in dichloromethane (20.0 mL) was added ethyl 2-ethoxy-2H-quinoline-1-carboxylate (38.8 g, 157 mmol, 3.00 eq). Then the mixture was stirred at 25° C. for 12 h. The mixture was concentrated to give a residue. The residue was triturated with ethyl acetate (100 mL) for twice. The filter cake was dried under reduced pressure to give (9H-fluoren-9-yl)methyl ((S)-1-(((S)-1-((4-(hydroxymethyl)-3-methoxyphenyl)amino)-1-oxopropan-2-yl)amino)-1-oxopropan-2-yl) carbamate Int 52b (21.0 g, crude) as a white solid. ¹H NMR (400 MHz, DMSO-d₆) δ=9.87 (s, 1H), 8.09 (br d, J=7.2 Hz, 1H), 7.89 (d, J=7.2 Hz, 2H), 7.72 (br t, J=8.4 Hz, 2H), 7.56 (br d, J=7.6 Hz, 1H), 7.45-7.38 (m, 2H), 7.36-7.29 (m, 3H), 7.25 (d, J=8.4 Hz, 1H), 7.14 (br d, J=9.2 Hz, 1H), 4.90 (t, J=5.6 Hz, 1H), 4.42 (d, J=5.6 Hz, 3H), 4.30-4.21 (m, 3H), 4.08 (br dd, J=7.2, 14.6 Hz, 1H), 3.70 (s, 3H), 1.31 (d, J=7.2 Hz, 3H), 1.25-1.22 (m, 3H). LCMS (ESI) m/z 540.2 [M+Na]f

### Step C. Preparation of Int 52c

To a solution of (9H-fluoren-9-yl)methyl ((S)-1-(((S)-1-((4-(hydroxymethyl)-3-methoxyphenyl)amino)-1-oxopropan-2-yl)amino)-1-oxopropan-2-yl)carbamate Int 52b (1.40 g, 2.70 mmol, 1.00 eq) in tetrahydrofuran (20.0 mL) was added phosphorus tribromide (1.46 g, 5.41 mmol, 2.00 eq). Then the mixture was stirred at 25° C. for 2 h. The mixture was poured into ice water (20.0 mL) and extracted with ethyl acetate (3×20 mL). The organic layer was dried over sodium sulfate and filtered. The filtrate was concentrated to give (9H-fluoren-9-yl)methyl ((S)-1-(((S)-1-((4-(bromomethyl)-3-methoxyphenyl)amino)-1-oxopropan-2-yl)amino)-1-oxopropan-2-yl)carbamate Int 52c (1.57 g, crude) as a yellow solid.

### Step D. Preparation of Int 52d

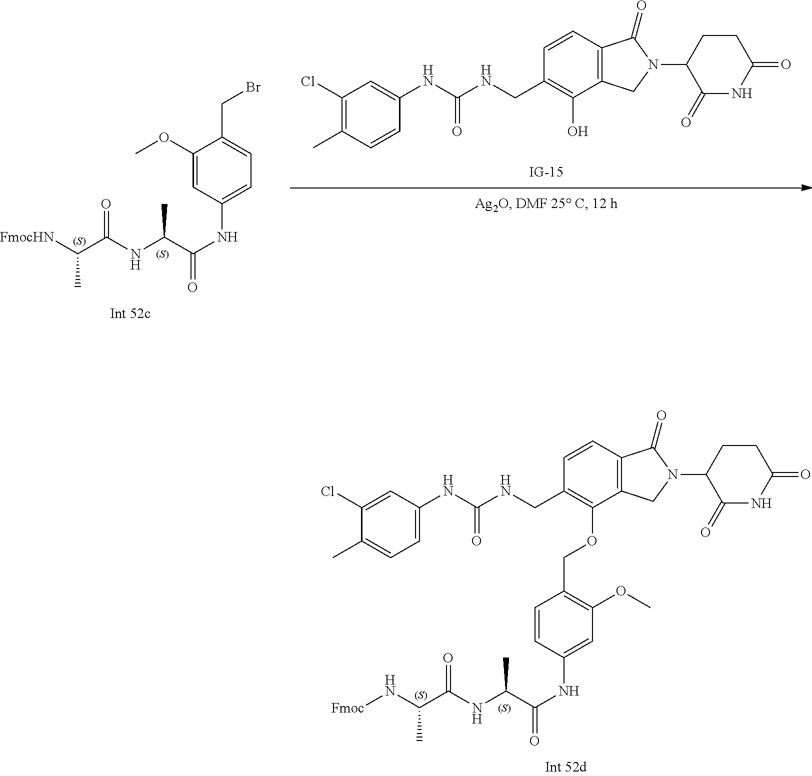

To a solution of 1-(3-chloro-4-methylphenyl)-3-((2-(2,6-dioxopiperidin-3-yl)-4-hydroxy-1-oxoisoindolin-5-yl)methyl)urea Int 52c (40.0 mg, 87.6 μmol, 1.00 eq) and (9H-fluoren-9-yl)methyl ((S)-1-(((S)-1-((4-(bromomethyl)-3-methoxyphenyl)amino)-1-oxopropan-2-yl)amino)-1-oxopropan-2-yl)carbamate (102 mg, 175 μmol, 2.00 eq) in dimethyl formamide (2.00 mL) was added silver oxide (101 mg, 438 μmol, 5.00 eq). Then the mixture was stirred at 25° C. for 12 h. The mixture was filtered. The filtrate was purified by reversed phase (C18, 330 g; condition: water/acetonitrile=100/0 to 0/100, 0.1% formic acid), further purified by reversed phase (C18, 80 g; condition: water/acetonitrile=100/0 to 0/100, 0.1% formic acid) and lyophilized to afford (9H-fluoren-9-yl)methyl ((2S)-1-(((2S)-1-((4-(((5-((3-(3-chloro-4-methylphenyl)ureido)methyl)-2-(2,6-dioxopiperidin-3-yl)-1-oxoisoindolin-4-yl)oxy)methyl)-3-methoxyphenyl)amino)-1-oxopropan-2-yl)amino)-1-oxopropan-2-yl)carbamate Int 52d (180 mg, 188 μmol, crude) as a yellow solid. LCMS (ESI) m/z 956.4 [M+H]⁺

### Step E. Preparation of Int 52e

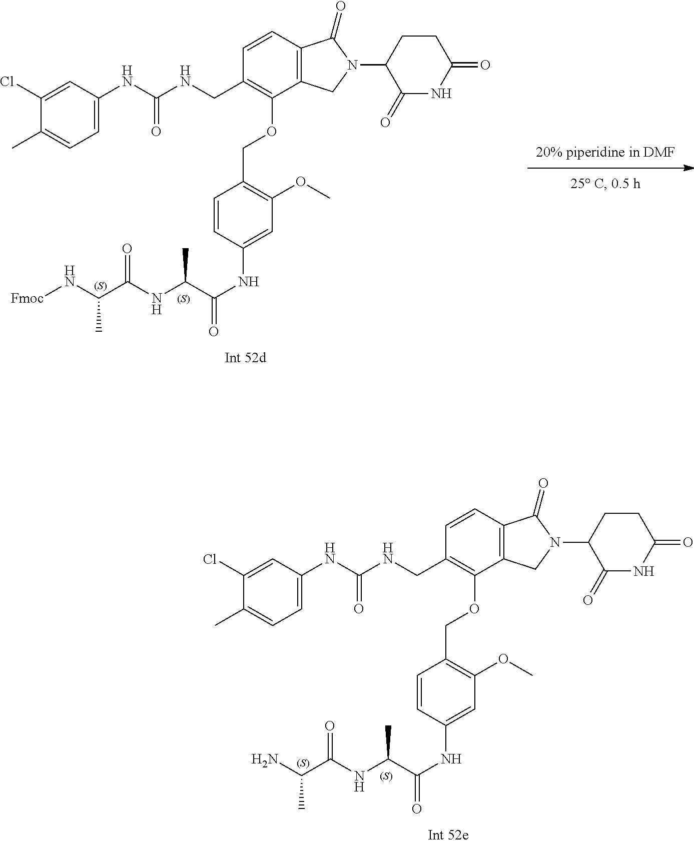

(9H-fluoren-9-yl)methyl ((2S)-1-(((2S)-1-((4-(((5-((3-(3-chloro-4-methylphenyl)ureido)methyl)-2-(2,6-dioxopiperidin-3-yl)-1-oxoisoindolin-4-yl)oxy)methyl)-3-methoxyphenyl)amino)-1-oxopropan-2-yl)amino)-1-oxopropan-2-yl)carbamate Int 52d (170 mg, 178 μmol, 1.00 eq) in piperidine (0.200 mL) and dimethyl formamide (0.800 mL) was stirred at 25° C. for 0.5 h. The mixture was filtered. The filtrate was purified by reversed phase (C18, 40 g; condition: water/acetonitrile=100/0 to 0/100, 0.1% formic acid), further purified by Prep-HPLC (column: Welch Ultimate C18 150*25 mm*5 um; mobile phase: [water (formic acid)-acetonitrile]; gradient: 20%-50% B over 40 min) and lyophilized to afford (2S)-2-amino-N-((2S)-1-((4-(((5-((3-(3-chloro-4-methylphenyl)ureido) methyl)-2-(2,6-dioxopiperidin-3-yl)-1-oxoisoindolin-4-yl)oxy)methyl)-3-methoxyphenyl)amino)-1-oxopropan-2-yl)propenamide Int 52e (15.0 mg, 20.4 μmol, 11% yield) as a white solid. ¹H NMR (400 MHz, DMSO-d₆) δ=10.95 (s, 1H), 10.12 (s, 1H), 8.28 (s, 2H), 7.67 (d, J=2.0 Hz, 1H), 7.43 (s, 1H), 7.35 (dd, J=2.0, 8.4 Hz, 1H), 7.19 (br d, J=8.8 Hz, 2H), 7.13-7.04 (m, 3H), 6.99 (br d, J=15.6 Hz, 1H), 5.08 (dd, J=4.8, 13.2 Hz, 1H), 4.47 (br d, J=5.6 Hz, 5H), 4.36-4.12 (m, 3H), 3.77 (s, 3H), 2.90 (br s, 1H), 2.64-2.55 (m, 1H), 2.40-2.31 (m, 1H), 2.24 (s, 3H), 2.03-1.95 (m, 1H), 1.31 (d, J=7.2 Hz, 3H), 1.20 (d, J=6.8 Hz, 3H). LCMS (ESI) m/z 734.3 [M+H]⁺

### Step F. Preparation of Compound IG-L-52

To a solution of ((2S)-2-amino-N-((2S)-1-((4-(((5-((3-(3-chloro-4-methylphenyl)ureido)methyl)-2-(2,6-dioxopiperidin-3-yl)-1-oxoisoindolin-4-yl)oxy) methyl)-3-methoxyphenyl)amino)-1-oxopropan-2-yl) propenamide Int 52e (14.0 mg, 19.1 μmol, 1.00 eq) and 34-(3-(2,5-dioxo-2,5-dihydro-1H-pyrrol-1-yl)-2-fluoro-6-(3-oxo-3-(2,3,5,6-tetrafluorophenoxy)propyl)phenoxy)-3,6,9,12,15,18,21,24,27,30-decamethyl-4,7,10,13,16, 19,22,25,28,31-decaoxo-3,6,9,12,15,18,21,24,27,30-decaazatetratriacontanoic acid Int 36k (23.3 mg, 19.1 μmol, 1.00 eq) in dimethyl formamide (1.00 mL) was added triethylamine (3.86 mg, 38.1 μmol, 5.31 μL, 2.00 eq). Then the mixture was stirred at 25° C. for 0.5 h. The mixture was filtered. The filtrate was purified by Prep-HPLC (column: UniSil 3-100 C18 Ultra (150*25 mm*3 um); mobile phase: [water (formic acid)-acetonitrile]; gradient: 20%-50% B over 40 min) and lyophilized to afford 34-(6-(3-(((2S)-1-(((2S)-1-((4-(((5-((3-(3-chloro-4-methylphenyl)ureido) methyl)-2-(2,6-dioxopiperidin-3-yl)-1-oxoisoindolin-4-yl)oxy) methyl)-3-methoxyphenyl)amino)-1-oxopropan-2-yl)amino)-1-oxopropan-2-yl)amino)-3-oxopropyl)-3-(2,5-dioxo-2,5-dihydro-11H-pyrrol-1-yl)-2-fluorophenoxy)-3,6,9,12,15,18,21,24,27,30-decamethyl-4,7,10,13,16, 19,22,25,28, 31-decaoxo-3,6,9,12,15,18,21,24,27,30-decaazatetratriacontanoic acid, Compound IG-L-52 (11.79 mg, 6.51 μmol, 34% yield, 99% purity) as a white solid. ¹H NMR (400 MHz, DMSO-d₆) δ=10.98 (s, 1H), 10.00-9.86 (m, 1H), 8.85-8.70 (m, 1H), 8.21-8.04 (m, 2H), 7.64 (d, J=2.0 Hz, 1H), 7.44 (s, 1H), 7.36-7.31 (m, 1H), 7.27-7.15 (m, 6H), 7.14-6.99 (m, 3H), 5.09 (dd, J=5.2, 13.1 Hz, 1H), 4.51 (br d, J=4.4 Hz, 3H), 4.42-4.14 (m, 15H), 4.13-3.89 (m, 13H), 3.75 (s, 3H), 3.04-2.69 (m, 36H), 2.57 (br d, J=2.0 Hz, 1H), 2.38 (br d, J=4.0 Hz, 1H), 2.24 (s, 3H), 2.08-1.88 (m, 3H), 1.30 (d, J=7.2 Hz, 3H), 1.19 (br d, J=6.8 Hz, 3H). LCMS (ESI) m/z 896.9 [M/2+2H]⁺

### Example L-53: Synthesis of34-(6-(3-(((2S)-1-(((2S)-1-((4-((((2-(3-(5-((3-(3-chloro-4-methylphenyl)ureido)methyl)-1-oxoisoindolin-2-yl)-2,6-dioxopiperidine-1-carbonyl)benzyl)(methyl)carbamoyl)oxy)methyl)phenyl)amino)-1-oxopropan-2-yl)amino)-1-oxopropan-2-yl)amino)-3-oxopropyl)-3-(2,5-dioxo-2,5-dihydro-1H-pyrrol-1-yl)-2-fluorophenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid (Compound IG-L-53)

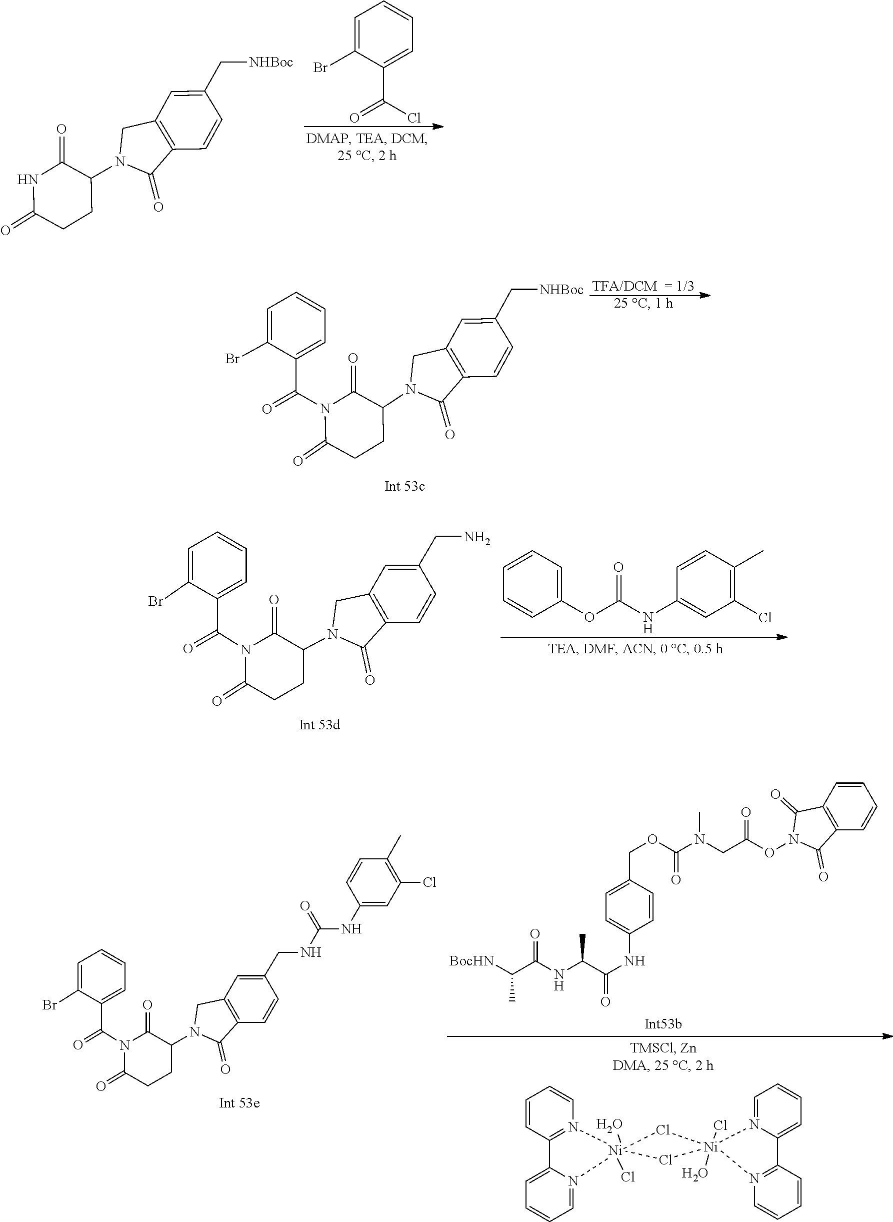

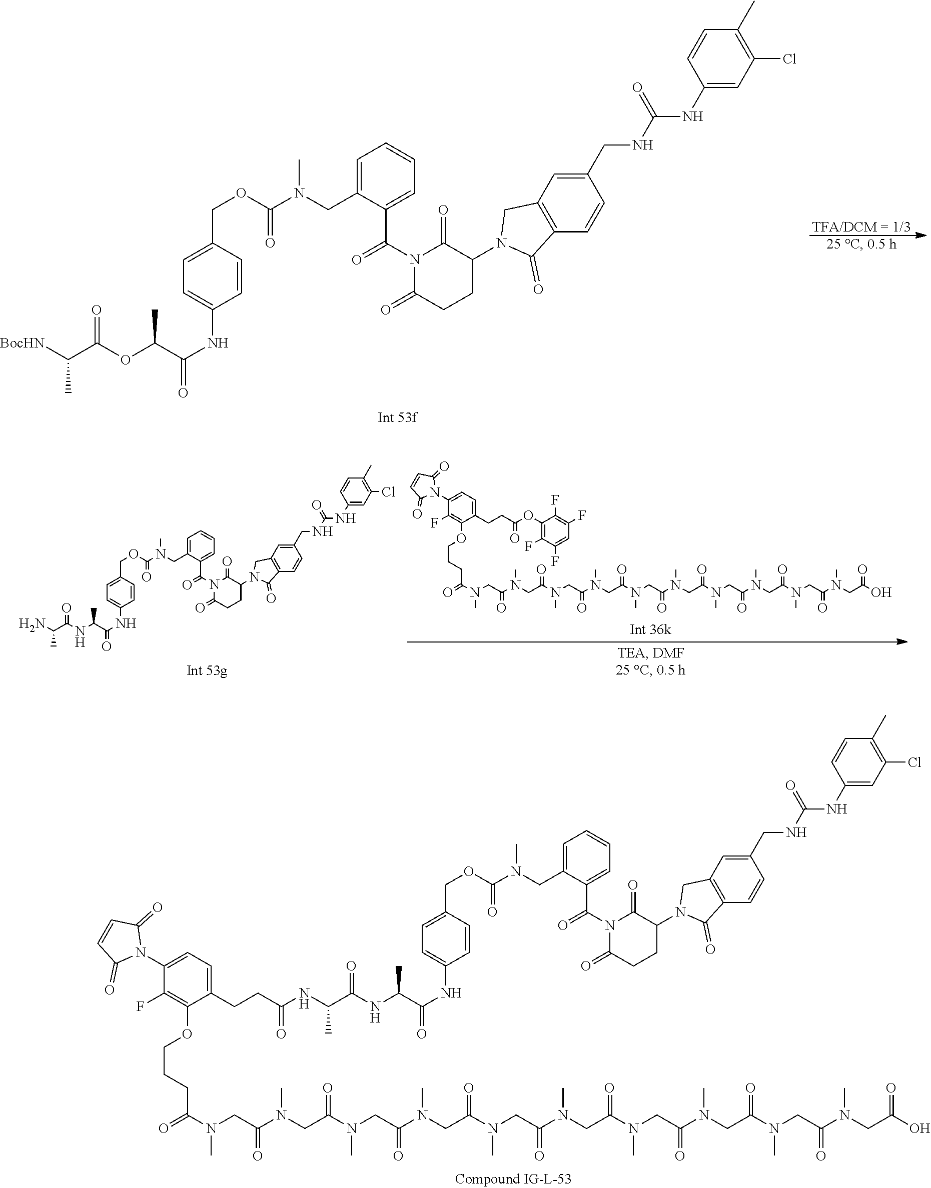

### Step A. Preparation of Int-53a

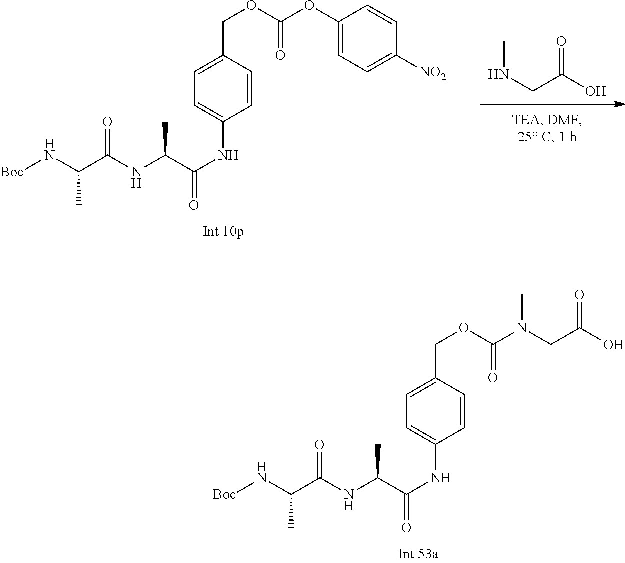

To a solution of tert-butyl ((S)-1-(((S)-1-((4-((((4-nitrophenoxy)carbonyl)oxy)methyl)phenyl)amino)-1-oxopropan-2-yl)amino)-1-oxopropan-2-yl)carbamate Int 10p (1.31 g, 2.47 mmol, 1.00 eq) and 2 methylglycine (264 mg, 2.96 mmol, 1.20 eq) in dimethyl formimade (10.0 mL) was added triethylamine (750 mg, 7.41 mmol, 1.03 mL, 3.00 eq). Then the mixture was stirred at 25° C. for 1 h. The mixture was filtered. The filtrate was purified by reversed phase chromatography (C18, 120 g, condition: water/acetonitrile=100/0 to 0/100, 0.1% formic acid) and lyophilized to afford N-(((4-((S)-2-((S)-2-((tert-butoxycarbonyl)amino)propanamido) propanamido)benzyl)oxy)carbonyl)-N-methylglycine Int 53a (1.10 g, 2.29 mmol, 92% yield) as a white solid. ¹H NMR (400 MHz, DMSO-d₆) δ=10.02 (br d, J=7.2 Hz, 1H), 8.21-8.10 (m, 2H), 8.04 (d, J=7.2 Hz, 1H), 7.33 (d, J=8.6 Hz, 1H), 6.95 (d, J=9.2 Hz, 2H), 5.02 (d, J=14.1 Hz, 2H), 4.48-4.34 (m, 1H), 4.01 (br t, J=7.2 Hz, 1H), 3.95 (d, J=5.9 Hz, 2H), 2.90 (d, J=10.3 Hz, 3H), 1.40 (s, 9H), 1.32 (d, J=7.1 Hz, 3H), 1.20 (br d, J=7.1 Hz, 3H).

### Step B. Preparation of Int 53b

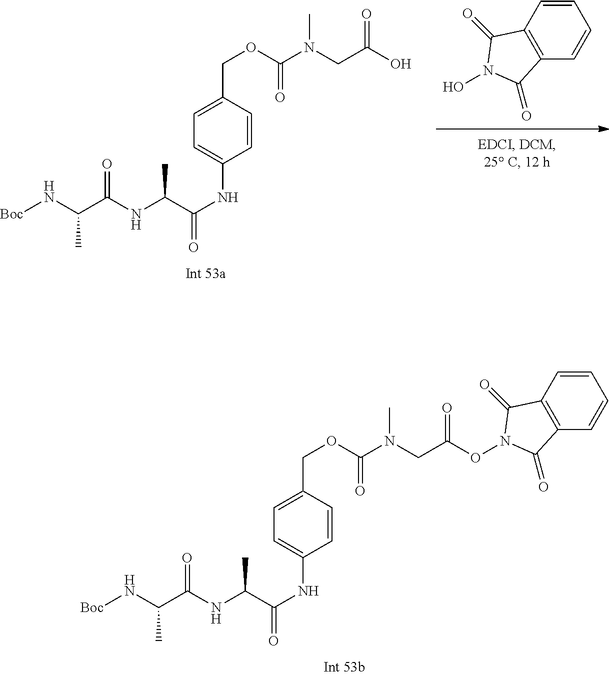 [SMILES: C[C@H](NC(=O)OC(C)(C)C)C(=O)N[C@@H](C)C(=O)Nc1ccc(COC(=O)N(C)CC(=O)O)cc1 ; O=C1c2ccccc2C(=O)N1O ; C[C@H](NC(=O)OC(C)(C)C)C(=O)N[C@@H](C)C(=O)Nc1ccc(COC(=O)N(C)CC(=O)ON2C(=O)c3ccccc3C2=O)cc1]

To a solution of N-(((4-((S)-2-((S)-2-((tert-butoxycarbonyl)amino)propanamido)propanamido)benzyl)oxy) carbonyl)-N-methylglycine Int 53a (1.10 g, 2.29 mmol, 1.00 eq) and 2-hydroxyisoindoline-1,3-dione (411 mg, 2.52 mmol, 1.10 eq) in dichloromethane (40.0 mL) was added 3-(ethyliminomethylideneamino)propyl-dimethylazanium;chloride (571 mg, 2.98 mmol, 1.30 eq) at 25° C. for 12 h. The mixture was filtered. The filtrate was purified by reversed phase chromatography (C18, 120 g; condition: water/acetonitrile=100/0 to 0/100, 0.1% formic acid) and lyophilized to afford 1,3-dioxoisoindolin-2-yl N-(((4-((S)-2-((S)-2-((tert-butoxycarbonyl)amino)propanamido)propanamido)benzyl)oxy)carbonyl)-N-methylglycinate Int 53b (1.35 g, 2.16 mmol, 94% yield) as a white solid. ¹H NMR (400 MHz, DMSO-d₆) δ=9.99 (br s, 1H), 8.04-7.90 (m, 4H), 7.57 (br d, J=7.6 Hz, 2H), 7.37-7.27 (m, 2H), 6.97 (br d, J=6.8 Hz, 1H), 5.05 (d, J=10.6 Hz, 2H), 4.61 (d, J=13.0 Hz, 1H), 4.46-4.31 (m, 2H), 3.98 (br t, J=7.0 Hz, 1H), 3.02-2.91 (m, 3H), 1.36 (s, 9H), 1.29 (br d, J=6.8 Hz, 3H), 1.17 (br d, J=6.8 Hz, 3H). LCMS (ESI) m/z 648.1 [M+Na]⁺

### Step C. Preparation of Int 53c

To a solution of tert-butyl ((2-(2,6-dioxopiperidin-3-yl)-1-oxoisoindolin-5-yl)methyl)carbamate (5.00 g, 13.4 mmol, 1.00 eq) in dichloromethane (100 mL) were added triethylamine (4.06 g, 40.2 mmol, 5.59 mL, 3.00 eq), dimethylaminopyridine (164 mg, 1.34 mmol, 0.100 eq) and 2-bromobenzoyl chloride (5.88 g, 26.8 mmol, 3.50 mL, 2.00 eq) at 25° C. Then the mixture was stirred at 25° C. for 12 h. The mixture was concentrated to give a residue. The residue was purified by reversed phase chromatography (C18, 330 g; condition: water/acetonitrile=100/0 to 0/100, 0.1% formic acid) and lyophilized to afford tert-butyl ((2-(1-(2-bromobenzoyl)-2,6-dioxopiperidin-3-yl)-1-oxoisoindolin-5-yl)methyl)carbamate Int 53c (5.00 g, 8.99 mmol, 67% yield) as a yellow solid. LCMS (ESI) m/z 556.1, 558.1 [M+H]⁺

### Step D. Preparation of Int 53d

To a solution of tert-butyl ((2-(1-(2-bromobenzoyl)-2,6-dioxopiperidin-3-yl)-1-oxoisoindolin-5-yl)methyl)carbamate Int 53c (3.00 g, 5.39 mmol, 1.00 eq) in dichloromethane (45.0 mL) was added trifluoroacetic acid (15.0 mL). Then the mixture was stirred at 25° C. for 1 h. The mixture was quenched with ice water (400 mL) and extracted with ethyl acetate (3×100 mL). The organic layer was dried over sodium sulfate and filtered. The filtrate was concentrated to give a residue. The residue was purified by reversed phase chromatography (C18, 330 g; condition: water/acetonitrile=100/0 to 0/100, 0.1% formic acid) and lyophilized to afford 3-(5-(aminomethyl)-1-oxoisoindolin-2-yl)-1-(2-bromobenzoyl)piperidine-2,6-dione Int 53d (2.20 g, 4.82 mmol, 89% yield) as a yellow solid. ¹H NMR (400 MHz, DMSO-d₆) δ=8.07-8.01 (m, 1H), 7.89-7.76 (m, 2H), 7.68 (s, 1H), 7.62-7.53 (m, 3H), 5.50 (dd, J=5.2, 13.2 Hz, 1H), 4.61-4.34 (m, 2H), 4.16 (s, 2H), 3.21 (br d, J=5.2 Hz, 1H), 2.97-2.82 (m, 1H), 2.71-2.61 (m, 1H), 2.22-2.13 (m, 1H). LCMS (ESI) m/z 456.0, 458.0 [M+Na]⁺

### Step E. Preparation of Int 53e

To a solution of 3-(5-(aminomethyl)-1-oxoisoindolin-2-yl)-1-(2-bromobenzoyl)piperidine-2,6-dione Int 53d (2.20 g, 4.82 mmol, 1.00 eq) and phenyl (3-chloro-4-methylphenyl)carbamate (1.89 g, 7.23 mmol, 1.50 eq) in dimethyl formamide (1.00 mL) and acetonitrile (5.00 mL) was added trimethylamine (1.46 g, 14.5 mmol, 2.01 mL, 3.00 eq) at 0° C. Then the mixture was stirred at 0° C. for 0.5 h. The mixture was diluted with ice water (200 mL) and extracted with ethyl acetate (3×100 mL). The organic layer was washed with brine (3×100 mL), dried over sodium sulfate and filtered. The filtrate was concentrated to give a residue. The residue was purified by reversed phase chromatography (C18, 330 g; condition: water/acetonitrile=100/0 to 0/100, 0.1% formic acid) and lyophilized to afford 1-((2-(1-(2-bromobenzoyl)-2,6-dioxopiperidin-3-yl)-1-oxoisoindolin-5-yl)methyl)-3-(3-chloro-4-methylphenyl)urea Int 53e (0.90 g, 1.44 mmol, 29% yield) as a white solid. ¹H NMR (400 MHz, DMSO-d₆) δ=8.74 (s, 1H), 8.10-7.99 (m, 1H), 7.91-7.81 (m, 1H), 7.71-7.63 (m, 2H), 7.57 (td, J=2.4, 4.6 Hz, 2H), 7.52 (s, 1H), 7.43 (br d, J=7.6 Hz, 1H), 7.22-7.09 (m, 2H), 6.78 (br t, J=6.0 Hz, 1H), 5.48 (dd, J=5.2, 13.2 Hz, 1H), 4.55-4.37 (m, 4H), 3.25-3.18 (m, 1H), 2.86 (br d, J=18.8 Hz, 1H), 2.64 (br dd, J=4.4, 13.4 Hz, 1H), 2.22 (s, 3H), 2.19-2.10 (m, 1H). LCMS (ESI) m/z 623.1, 625.1 [M+H]⁺

### Step F. Preparation of Int 53f

To a solution of 1-((2-(1-(2-bromobenzoyl)-2,6-dioxopiperidin-3-yl)-1-oxoisoindolin-5-yl)methyl)-3-(3-chloro-4-methylphenyl)urea Int 53e (400 mg, 641 gmol, 1.00 eq) and (1,3-dioxoisoindolin-2-yl N-(((4-((S)-2-((S)-2-((tert-butoxycarbonyl)amino)propanamido)propanamido)benzyl)oxy)carbonyl)-N-methylglycinate Int 53b (441 mg, 705 μmol, 1.10 eq) in dimethylacetamide (10.0 mL) were added dichloronickel; 2-(2-pyridyl)pyridine;dihydrate (38.9 mg, 64.1 μmol, 0.10 eq), zinc powder (380 mg, 5.81 mmol, 9.06 eq) and trimethylchlorosilane (209 mg, 1.92 mmol, 244 μL, 3.00 eq) at 25° C. Then the mixture was stirred at 25° C. for 2 h. The mixture was filtered. The filtrate was diluted with water (100 mL) and extracted with ethyl acetate (3×200 mL). The organic layer was concentrated to give a residue. The residue was purified by reversed phase chromatography (C18, 40 g; condition: water/acetonitrile=100/0 to 0/100, 0.1% formic acid) and lyophilized to afford 4-((S)-2-((S)-2-((tert-butoxycarbonyl)amino)propanamido)propanamido)benzyl (2-(3-(5-((3-(3-chloro-4-methylphenyl)ureido)methyl)-1-oxoisoindolin-2-yl)-2,6-dioxopiperidine-1-carbonyl) benzyl)(methyl)carbamate Int 53f (150 mg, 153 μmol, 23% yield) as a white solid. ¹H NMR (400 MHz, DMSO-d₆) δ=10.08-9.87 (m, 1H), 8.75 (s, 1H), 8.11-7.93 (m, 2H), 7.73-7.57 (m, 4H), 7.54-7.41 (m, 4H), 7.39-7.22 (m, 2H), 7.20-7.08 (m, 3H), 7.00-6.92 (m, 1H), 6.79 (t, J=5.6 Hz, 1H), 5.50 (br dd, J=5.2, 13.6 Hz, 1H), 5.12-4.95 (m, 2H), 4.91-4.79 (m, 1H), 4.78-4.68 (m, 1H), 4.57-4.33 (m, 5H), 4.04-3.94 (m, 1H), 2.93-2.82 (m, 4H), 2.67-2.60 (m, 1H), 2.47-2.38 (m, 1H), 2.23 (s, 3H), 2.20-2.13 (m, 1H), 1.43-1.13 (m, 15H). LCMS (ESI) m/z 879.5 [M+H-100]⁺

### Step G. Preparation of Int 53g

To a solution of 4-((S)-2-((S)-2-((tert-butoxycarbonyl)amino)propanamido)propanamido)benzyl(2-(3-(5-((3-(3-chloro-4-methylphenyl)ureido)methyl)-1-oxoisoindolin-2-yl)-2,6-dioxopiperidine-1-carbonyl)benzyl) (methyl)carbamate (150 mg, 153 μmol, 1.00 eq) in dichloromethane (0.900 mL) was added trifluoroacetic acid (0.300 mL). Then the mixture was stirred at 25° C. for 0.5 h. The mixture was filtered. The filtrate was purified by reversed phase chromatography (C18, 80 g; condition: water/acetonitrile=100/0 to 0/100, 0.1% formic acid) and prep-HPLC (column: Phenomenex luna C18 150*25 mm*10 um; mobile phase: [water (formic acid)-acetonitrile]; gradient: 20%-50% B over 15 min) and lyophilized to afford 4-((S)-2-((S)-2-aminopropanamido)propanamido)benzyl(2-(3-(5-((3-(3-chloro-4-methylphenyl)ureido)methyl)-1-oxoisoindolin-2-yl)-2,6-dioxopiperidine-1-carbonyl)benzyl)(methyl)carbamate (30.0 mg, 34.1 mol, 22% yield) as a white solid. LCMS (ESI) m/z 879.5 [M+H]⁺

### Step H. Preparation of Compound IG-L-53

To a solution of 4-((S)-2-((S)-2-aminopropanamido)propanamido)benzyl(2-(3-(5-((3-(3-chloro-4-methylphenyl)ureido)methyl)-1-oxoisoindolin-2-yl)-2,6-dioxopiperidine-1-carbonyl)benzyl)(methyl) carbamate Int 53g (20.0 mg, 22.7 μmol, 1.00 eq) and 34-(3-(2,5-dioxo-2,5-dihydro-1H-pyrrol-1-yl)-2-fluoro-6-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid Int 36k (27.8 mg, 22.7 μmol, 1.00 eq) in dimethyl formamid (1.00 mL) was added triethylamine (4.60 mg, 45.5 μmol, 6.33 μL, 2.00 eq). Then the mixture was stirred at 25° C. for 0.5 h. The mixture was filtered. The filtrate was purified by prep-HPLC (column: Welch Xtimate C18 150*25 mm*5 um; mobile phase: [water (formic acid)-acetonitrile]; gradient: 30%-60% B over 35 min) and lyophilized to afford 34-(6-(3-(((2S)-1-(((25)-1-((4-((((2-(3-(5-((3-(3-chloro-4-methylphenyl)ureido)methyl)-1-oxoisoindolin-2-yl)-2,6-dioxopiperidine-1-carbonyl)benzyl) (methyl) carbamoyl)oxy)methyl)phenyl)amino)-1-oxopropan-2-yl)amino)-1-oxopropan-2-yl)amino)-3-oxopropyl)-3-(2,5-dioxo-2,5-dihydro-1H-pyrrol-1-yl)-2-fluorophenoxy)-3,6,9,12,15,18,21,24,27,30-decamethyl-4,7,10,13, 16,19,22,25,28,31-decaoxo-3,6,9,12,15,18,21,24,27,30-decaazatetratriacontanoic acid, Compound IG-L-53 (9.00 mg, 4.60 mol, 20% yield, 96% purity) as a white solid. ¹H NMR (400 MHz, DMSO-d₆) δ=9.99-9.89 (m, 1H), 9.03-8.79 (m, 1H), 8.24-8.10 (m, 2H), 8.07 (br d, J=7.6 Hz, 1H), 7.72-7.61 (m, 4H), 7.53-7.45 (m, 3H), 7.43 (br d, J=8.0 Hz, 1H), 7.37-7.32 (m, 1H), 7.28-7.22 (m, 3H), 7.19-7.10 (m, 4H), 7.04 (br t, J=7.6 Hz, 1H), 6.93 (ddd, J=5.2, 8.4, 11.2 Hz, 1H), 5.56-5.45 (m, 1H), 5.10-4.97 (m, 2H), 4.91-4.81 (m, 1H), 4.78-4.68 (m, 1H), 4.58-4.48 (m, 1H), 4.40-4.28 (m, 10H), 4.25-4.16 (m, 5H), 4.10-3.89 (m, 13H), 2.95-2.68 (m, 40H), 2.22 (s, 3H), 2.20-2.11 (m, 2H), 1.96-1.88 (m, 2H), 1.32-1.27 (m, 3H), 1.18 (br s, 3H). LCMS (ESI) m/z 1938.1 [M+2H]⁺

### Example L-63: Synthesis of 1-(3-chloro-5-(2,5-dioxo-2,5-dihydro-1H-pyrrol-1-yl)-4-methylphenyl)-3-((2-(2,6-dioxopiperidin-3-yl)-1-oxoisoindolin-5-yl)methyl)urea, IG-L-63

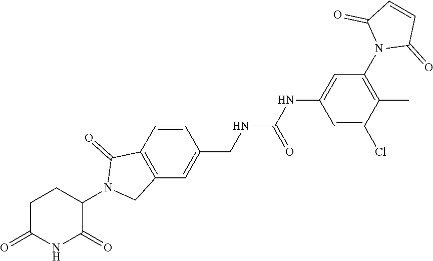 [SMILES: Cc1c(Cl)cc(NC(=O)NCc2ccc3c(c2)CN(C2CCC(=O)NC2=O)C3=O)cc1N1C(=O)C=CC1=O]

### Step A. Preparation of Int 63a

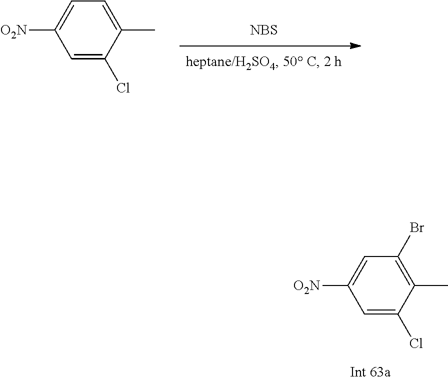

To a solution of 2-chloro-1-methyl-4-nitrobenzene (5.00 g, 29.1 mmol, 6.02 mL, 1.00 eq) in sulfuric acid (20.0 mL) and heptane (20.0 mL) was added N-bromosuccinimide (6.22 g, 34.9 mmol, 1.20 eq) in batches at 50° C. The mixture was stirred at 50° C. for 2 h. The mixture was dropwised into ice water (60 mL) and extracted with ethyl acetate (3×60 mL). The combined organic layers were dried over sodium sulfate and filtered. The filtrate was concentrated under reduced pressure to give a residue. The residue was purified by reversed-phase column (C18, 120 g; condition: water/acetonitrile=1/0-0/1, 0.1% formic acid) and lyophilized to afford 1-bromo-3-chloro-2-methyl-5-nitrobenzene Int 63a (1.80 g, 7.19 mmol, 24% yield) as a yellow solid. ¹H NMR (400 MHz, DMSO-d₆) δ=8.39 (d, J=2.4 Hz, 1H), 8.31 (d, J=2.4 Hz, 1H), 2.56 (s, 3H).

### Step B. Preparation of Int 63b

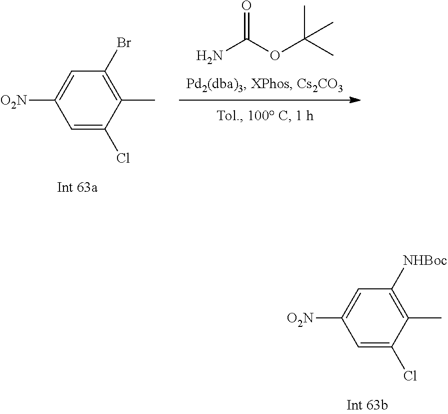

To a solution of 1-bromo-3-chloro-2-methyl-5-nitrobenzene Int 63a (1.00 g, 3.99 mmol, 1.00 eq) and tert-butyl carbamate (468 mg, 3.99 mmol, 1.00 eq) in toluene (10.0 mL) was added cesium carbonate (3.90 g, 12.0 mmol, 3.00 eq), 2-(dicyclohexylphosphino)-2,4,6-tri-i-propyl-1,1-biphenyl (381 mg, 798 μmol, 0.200 eq) and tris(dibenzylideneacetone)dipalladium(0) (366 mg, 399 μmol, 0.100 eq). The mixture was stirred at 100° C. for 1 h under nitrogen atmosphere. The mixture was concentrated under reduced pressure to give a residue. The residue was purified by reversed-phase column (C18, 120 g; condition: water/acetonitrile=1/0-0/1, 0.1% formic acid) and lyophilized to afford tert-butyl (3-chloro-2-methyl-5-nitrophenyl)carbamate Int 63b (420 mg, 1.17 mmol, 29% yield, 80% purity) as a yellow solid. ¹H NMR (400 MHz, DMSO-d₆) δ=9.23 (s, 1H), 8.32-8.30 (m, 1H), 8.05-8.03 (m, 1H), 2.34 (s, 3H), 1.49 (s, 9H).

### Step C. Preparation of Int 63c

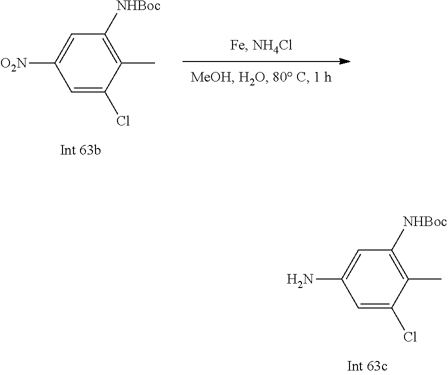

To a solution of tert-butyl (3-chloro-2-methyl-5-nitrophenyl)carbamate Int 63b (420 mg, 1.17 mmol, 1.00 eq) in methanol (20.0 mL) was added saturated ammonium chloride (188 mg, 3.52 mmol, 3.00 eq) in water (2.00 mL). Iron powder (327 mg, 5.86 mmol, 5.00 eq) was added to the mixture at 80° C. The mixture was stirred at 80° C. for 1 h. The mixture was filtered to give a filtrate and concentrated under reduced pressure to give a residue. The residue was purified by reversed-phase column (C18, 80 g; condition: water/acetonitrile=1/0-0/1, 0.1% formic acid) and lyophilized to afford tert-butyl (5-amino-3-chloro-2-methylphenyl)carbamate Int 63c (53.0 mg, 206 μmol, 17% yield) as a white solid. ¹H NMR (400 MHz, DMSO-d₆) δ=8.47 (s, 1H), 6.53 (d, J=2.0 Hz, 1H), 6.44 (d, J=2.0 Hz, 1H), 5.15 (s, 2H), 2.02 (s, 3H), 1.44 (s, 9H).

### Step D. Preparation of Int 63d

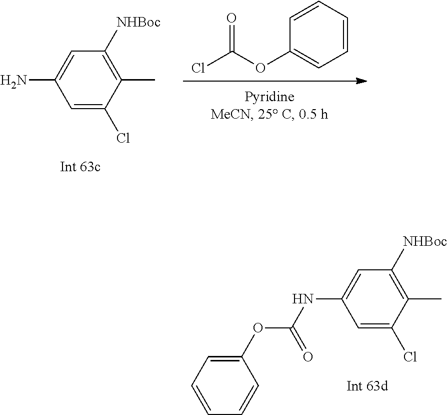 [SMILES: Cc1c(Cl)cc(N)cc1NC(=O)OC(C)(C)C ; O=C(Cl)Oc1ccccc1 ; Cc1c(Cl)cc(NC(=O)Oc2ccccc2)cc1NC(=O)OC(C)(C)C]

To a solution of tert-butyl (5-amino-3-chloro-2-methylphenyl)carbamate Int 63c (53.0 mg, 206 μmol, 1.00 eq) in acetonitrile (1.00 mL) was added phenyl carbonochloridate (32.3 mg, 206 μmol, 25.9 μL, 1.00 eq) and pyridine (32.7 mg, 413 μmol, 33.3 μL, 2.00 eq). The mixture was stirred at 25° C. for 0.5 h. The mixture was concentrated under reduced pressure to give a residue. The residue was purified by reversed-phase column (C18, 12 g; condition: water/acetonitrile=1/0-0/1, 0.1% formic acid) and lyophilized to afford tert-butyl phenyl (5-chloro-4-methyl-1,3-phenylene)dicarbamate Int 63d (55.0 mg, 144 μmol, 70% yield, 99% purity) as a white solid. MS (ESI) m/z 399.2 [M+Na]⁺

### Step E. Preparation of Int 63e

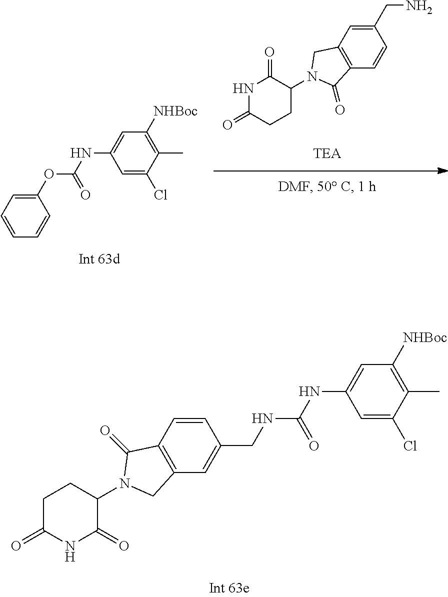 [SMILES: Cc1c(Cl)cc(NC(=O)Oc2ccccc2)cc1NC(=O)OC(C)(C)C ; NCc1ccc2c(c1)CN(C1CCC(=O)NC1=O)C2=O ; Cc1c(Cl)cc(NC(=O)NCc2ccc3c(c2)CN(C2CCC(=O)NC2=O)C3=O)cc1NC(=O)OC(C)(C)C]

To a solution of 3-(5-(aminomethyl)-1-oxoisoindolin-2-yl)piperidine-2,6-dione Int 63d (42.9 mg, 146 μmol, 1.00 eq) in N,N-dimethylformamide (1.00 mL) was added triethylamine (73.8 mg, 729 μmol, 102 μL, 5.00 eq) and tert-butyl phenyl (5-chloro-4-methyl-1,3-phenylene)dicarbamate (55.0 mg, 146 μmol, 1.00 eq). The mixture was stirred at 50° C. for 1 h. The mixture was concentrated under reduced pressure to give a residue. The residue was purified by reversed-phase column (C18, 40 g; condition: water/acetonitrile=1/0-0/1, 0.1% formic acid) and lyophilized to afford tert-butyl (3-chloro-5-(3-((2-(2,6-dioxopiperidin-3-yl)-1-oxoisoindolin-5-yl)methyl)ureido)-2-methylphenyl)carbamate Int 63e (45.0 mg, 80.9 gmol, 55% yield) as a white solid. ¹H NMR (400 MHz, DMSO-d₆) δ=10.97 (s, 1H), 8.81 (s, 1H), 8.71-8.68 (m, 1H), 7.69 (d, J=8.0 Hz, 1H), 7.52-7.50 (m, 2H), 7.43 (d, J=8.4 Hz, 1H), 7.26 (d, J=2.0 Hz, 1H), 6.73 (t, J=6.0 Hz, 1H), 5.10 (dd, J=5.2, 13.2 Hz, 1H), 4.49-4.28 (m, 4H), 2.97-2.85 (m, 1H), 2.57 (br s, 1H), 2.44-2.35 (m, 1H), 2.12-2.11 (m, 3H), 2.04-1.94 (m, 1H), 1.45 (s, 9H). MS (ESI) m/z 500.2 [M-56+H]⁺

### Step F. Preparation of Int 63f

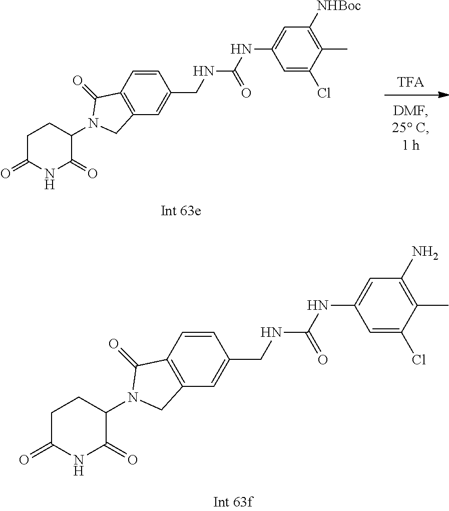 [SMILES: Cc1c(Cl)cc(NC(=O)NCc2ccc3c(c2)CN(C2CCC(=O)NC2=O)C3=O)cc1NC(=O)OC(C)(C)C ; "TFA" ; Cc1c(N)cc(NC(=O)NCc2ccc3c(c2)CN(C2CCC(=O)NC2=O)C3=O)cc1Cl]

To a solution of tert-butyl (3-chloro-5-(3-((2-(2,6-dioxopiperidin-3-yl)-1-oxoisoindolin-5-yl)methyl)ureido)-2-methylphenyl)carbamate Int 63e (45.0 mg, 80.9 μmol, 1.00 eq) in dichloromethane (1.00 mL) was added trifluoroacetic acid (307 mg, 2.69 mmol, 0.200 mL, 33.3 eq). The mixture was stirred at 25° C. for 1 h. The mixture was concentrated under reduced pressure to give a residue. The residue was purified by reversed-phase column (C18, 40 g; condition: water/acetonitrile=1/0-0/1, 0.1% formic acid) and lyophilized to afford 1-(3-amino-5-chloro-4-methylphenyl)-3-((2-(2,6-dioxopiperidin-3-yl)-1-oxoisoindolin-5-yl)methyl)urea Int 63f (27.0 mg, 56.3 μmol, 69% yield, 95% purity) as a white solid. MS (ESI) m/z 456.1 [M+H]⁺

### Step G. Preparation of Compound IG-L-63

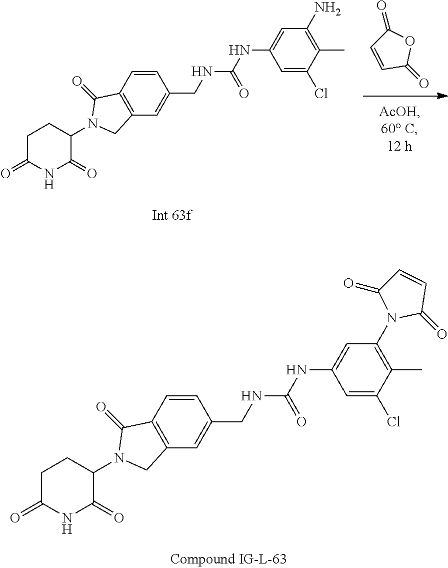

To a solution of 1-(3-amino-5-chloro-4-methylphenyl)-3-((2-(2,6-dioxopiperidin-3-yl)-1-oxoisoindolin-5-yl)methyl)urea Int 63f (17.0 mg, 37.3 μmol, 1.00 eq) in acetic acid (1.00 mL) was added furan-2,5-dione (3.66 mg, 37.3 μmol, 1.00 eq). The mixture was stirred at 60° C. for 12 h. The mixture was concentrated under reduced pressure to give a residue. The residue was purified by reversed-phase column (C18, 40 g; condition: water/acetonitrile=1/0-0/1, 0.1% formic acid) and lyophilized to afford 1-(3-chloro-5-(2,5-dioxo-2,5-dihydro-1H-pyrrol-1-yl)-4-methylphenyl)-3-((2-(2,6-dioxopiperidin-3-yl)-1-oxoisoindolin-5-yl)methyl)urea, Compound IG-L-63 (3.82 mg, 7.06 μmol, 19% yield, 99% purity) as a white solid. ¹H NMR (400 MHz, DMSO-d₆) δ=10.97 (br s, 1H), 9.15-8.97 (m, 1H), 7.75-7.64 (m, 2H), 7.51 (s, 1H), 7.43 (d, J=8.0 Hz, 1H), 7.25 (d, J=2.0 Hz, 1H), 7.21 (s, 2H), 7.03 (br d, J=4.8 Hz, 1H), 5.10 (dd, J=5.2, 13.3 Hz, 1H), 4.47-4.28 (m, 4H), 2.97-2.83 (m, 1H), 2.63-2.56 (m, 1H), 2.44-2.35 (m, JH), 1.99 (s, 4H). MS (ESI) m/z 536.3 [M+H]⁺

The compounds in Table 20 were prepared in a manner similar to that described for Compound IG-L-63 using the appropriate compound as starting material.

TABLE 20

| IG-L | | |
|---|---|---|
| No. | Structure | ¹H NMR/MS |
| IG-L- 64 | 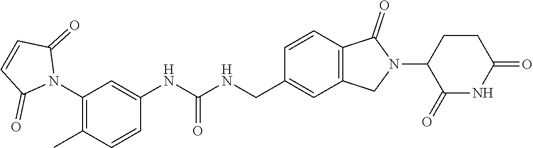 [SMILES: Cc1ccc(NC(=O)NCc2ccc3c(c2)CN(C2CCC(=O)NC2=O)C3=O)cc1N1C(=O)C=CC1=O] | ¹H NMR (400 MHz, DMSO-d₆) δ = 10.97 (s, 1H), 8.74 (s, 1H), 7.68 (d, J = 8.0 Hz, 1H), 7.51 (s, 1H), 7.43 (d, J = 8.0 Hz, 1H), 7.36 (d, J = 2.0 Hz, 1H), 7.33-7.29 (m, 1H), 7.23-7.18 (m, 3H), 6.77 (t, J = 6.0 Hz, 1H), 5.10 (dd, J = 5.2, 13.2 Hz, 1H), 4.47-4.42 (m, 1H), 4.40 (br d, J = 6.0 Hz, 2H), 4.34-4.26 (m, 1H), 2.96-2.86 (m, 1H), 2.59 (br d, J = 16.8 Hz, 1H), 2.38 (dq, J = 4.0, 13.2 Hz, 1H), 2.03-1.98 (m, 1H), 1.96 (s, 3H). MS (ESI) m/z 502.3 [M + H]⁺ |
| IG-L- 65 | 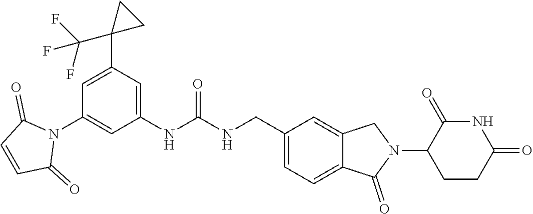 [SMILES: O=C1CCC(N2Cc3cc(CNC(=O)Nc4cc(N5C(=O)C=CC5=O)cc(C5(C(F)(F)F)CC5)c4)ccc3C2=O)C(=O)N1] | ¹H NMR (400 MHz, DMSO-d₆) δ = 10.97 (br s, 1H), 9.01 (s, 1H), 7.69 (d, J = 8.0 Hz, 1H), 7.60 (s, 1H), 7.51 (s, 1H), 7.46- 7.42 (m, 2H), 7.16 (s, 2H), 6.98 (s, 1H), 6.82 (t, J = 6.0 Hz, 1H), 5.10 (dd, J = 5.2, 13.6 Hz, 1H), 4.47-4.40 (m, 3H), 4.34- 4.27 (m, 1H), 2.96-2.86 (m, 1H), 2.61 (br d, J = 2.4 Hz, 1H), 2.40-2.33 (m, 1H), 2.02-1.95 (m, 1H), 1.36-1.31 (m, 2H), 1.09 (br s, 2H). MS (ESI) m/z 596.2 [M + H]⁺ |
| IG-L- 69 | 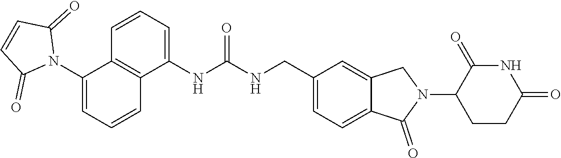 [SMILES: O=C1CCC(N2Cc3cc(CNC(=O)Nc4cccc5c(N6C(=O)C=CC6=O)cccc45)ccc3C2=O)C(=O)N1] | ¹H NMR (400 MHz, DMSO-d₆) δ = 11.01- 10.94 (m, 1H), 8.82 (s, 1H), 8.24 (d, J = 8.8 Hz, 1H), 8.02 (d, J = 7.6 Hz, 1H), 7.72 (d, J = 8.0 Hz, 1H), 7.68-7.63 (m, 1H), 7.58 (s, 1H), 7.56-7.42 (m, 2H), 7.32 (s, 1H), 7.29 (s, 2H), 7.18 (br t, J = 6.4 Hz, 1H), 5.16-5.05 (m, 1H), 4.50-4.35 (m, 3H), 2.95-2.88 (m, 1H), 2.69-2.61 (m, 1H), 2.38-2.31 (m, 1H), 2.04-1.97 (m, 1H). MS (ESI) m/z 538.3 [M + H]⁺ |

### Example L-67: Synthesis of 1-(5-(2-(2-(2,5-dioxo-2,5-dihydro-1H-pyrrol-1-yl)ethoxy)ethyl)naphthalen-1-yl)-3-((2-(2,6-dioxopiperidin-3-yl)-4-hydroxy-1-oxoisoindolin-5-yl)methyl)urea, IG-L-67

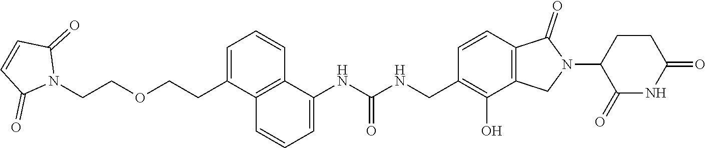 [SMILES: O=C1CCC(N2Cc3c(ccc(CNC(=O)Nc4cccc5c(CCOCCN6C(=O)C=CC6=O)cccc45)c3O)C2=O)C(=O)N1]

### Step A. Preparation of Int 67a

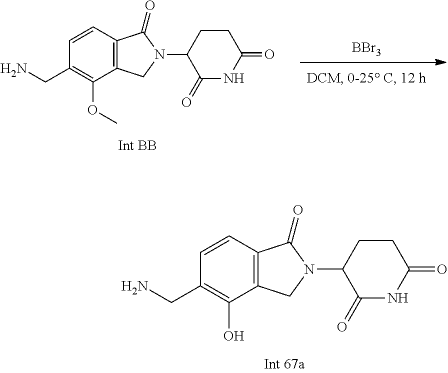 [SMILES: COc1c(CN)ccc2c1CN(C1CCC(=O)NC1=O)C2=O ; "BBr3" ; NCc1ccc2c(c1O)CN(C1CCC(=O)NC1=O)C2=O]

To a solution of 3-(5-(aminomethyl)-4-methoxy-1-oxoisoindolin-2-yl)piperidine-2,6-dione (200 mg, 659 μmol, 1.00 eq) in dichloromethane (5.00 mL) was added boron tribromide (3.30 g, 13.2 mmol, 1.27 mL, 20.0 eq) dropwise at 0° C. The mixture was stirred at 25° C. for 12 h. The reaction mixture was quenched with water (10 mL), filtered and the filtrate was concentrated under reduced pressure to give a crude product. The crude product was purified by reversed phase (0.1% formic acid condition) and lyophilized to afford 3-(5-(aminomethyl)-4-hydroxy-1-oxoisoindolin-2-yl)piperidine-2,6-dione Int 67a (150 mg, 519 μmol, 79% yield) as a brown solid. ¹H NMR (400 MHz, DMSO-d₆) δ=11.00 (s, 1H), 10.36 (br s, 1H), 8.08 (br s, 2H), 7.48 (d, J=7.6 Hz, 1H), 7.28 (d, J=7.6 Hz, 1H), 5.11 (dd, J=4.8, 13.2 Hz, 1H), 4.43-4.24 (m, 2H), 4.15-4.07 (m, 2H), 3.00-2.87 (m, 1H), 2.61 (br d, J=17.6 Hz, 1H), 2.36 (br dd, J=4.4, 13.2 Hz, 1H), 2.08-1.98 (m, 1H).

### Step B. Preparation of Int 67b

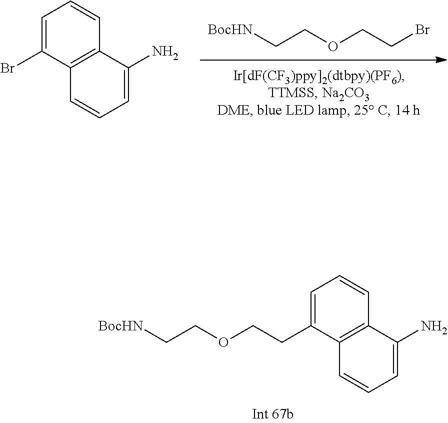 [SMILES: Nc1cccc2c(Br)cccc12 ; CC(C)(C)OC(=O)NCCOCCBr ; CC(C)(C)OC(=O)NCCOCCc1cccc2c(N)cccc12]

A mixture of tert-butyl (2-(2-bromoethoxy)ethyl)carbamate (157 mg, 585 μmol, 1.30 eq), 5-bromonaphthalen-1-amine (100 mg, 450 μmol, 1.00 eq), (4,4-di-tert-butyl-2,2-bipyridine) bis(3,5-difluoro-2-(5-trifluoromethyl-2-pyridinyl-kappa)phenyl-kappa) iridium(III) hexafluorophosphate (5.05 mg, 4.50 μmol, 0.0100 eq), bis(trimethylsilyl)silyl-trimethyl-silane (112 mg, 450 μmol, 139 μL, 1.00 eq) and sodium carbonate (95.5 mg, 901 μmol, 2.00 eq) in dimethyl ether (10.0 mL) was degassed and purged with nitrogen atmosphere. The mixture was stirred at 25° C. for 14 h inradiated with a 455 nm blue LED. The mixture was filtered and concentrated to afford a residue. The residue was purified by reversed phase (0.1% formic acid condition) and lyophilized to afford tert-butyl (2-(2-(5-aminonaphthalen-1-yl)ethoxy)ethyl)carbamate Int 67b (800 mg, 2.42 mmol, 67% yield) as brown oil. ¹H NMR (400 MHz, DMSO-d₆) δ=7.96-7.91 (m, 1H), 7.36-7.22 (m, 3H), 6.75 (br d, J=6.8 Hz, 2H), 3.72-3.65 (m, 2H), 3.59-3.54 (m, 1H), 3.46-3.40 (m, 4H), 3.20 (br t, J=7.2 Hz, 2H), 3.10-3.05 (m, 2H), 1.37 (s, 9H).

### Step C. Preparation of Int 67c

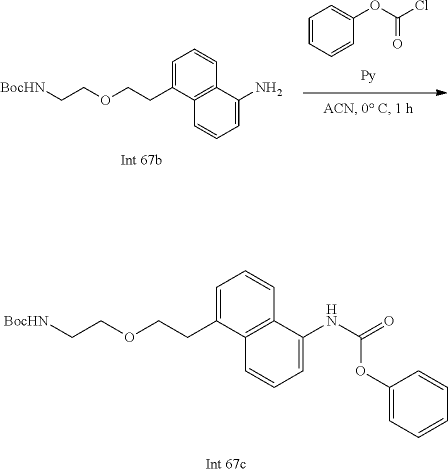 [SMILES: CC(C)(C)OC(=O)NCCOCCc1cccc2c(N)cccc12 ; O=C(Cl)Oc1ccccc1 ; CC(C)(C)OC(=O)NCCOCCc1cccc2c(NC(=O)Oc3ccccc3)cccc12]

To a solution of tert-butyl (2-(2-(5-aminonaphthalen-1-yl)ethoxy)ethyl)carbamate Int 67b (800 mg, 2.42 mmol, 1.00 eq) and pyridine (383 mg, 4.84 mmol, 391 μL, 2.00 eq) in acetonitrile (10.0 mL) was added phenyl carbonochloridate (758 mg, 4.84 mmol, 607 μL, 2.00 eq) dropwise at 0° C. The mixture was stirred at 0° C. for 1 h. The mixture was concentrated to afford a residue. The residue was purified by reversed phase (0.1% formic acid condition) and lyophilized to afford tert-butyl (2-(2-(5-((phenoxycarbonyl)amino)naphthalen-1-yl)ethoxy)ethyl)carbamate Int 67c (660 mg, 1.46 mmol, 61% yield) as a yellow solid. ¹H NMR (400 MHz, DMSO-d₆) δ=10.08 (br s, 1H), 8.06 (d, J=8.4 Hz, 1H), 7.99 (br d, J=8.4 Hz, 1H), 7.66 (d, J=7.2 Hz, 1H), 7.56 (d, J=7.6 Hz, 1H), 7.49 (d, J=8.4 Hz, 1H), 7.47-7.43 (m, 3H), 7.26 (br d, J=7.2 Hz, 3H), 6.75 (br t, J=5.2 Hz, 1H), 3.71 (t, J=7.2 Hz, 2H), 3.45-3.40 (m, 2H), 3.31 (br s, 2H), 3.08 (s, 2H), 1.38 (s, 9H).

### Step D. Preparation of Int 67d

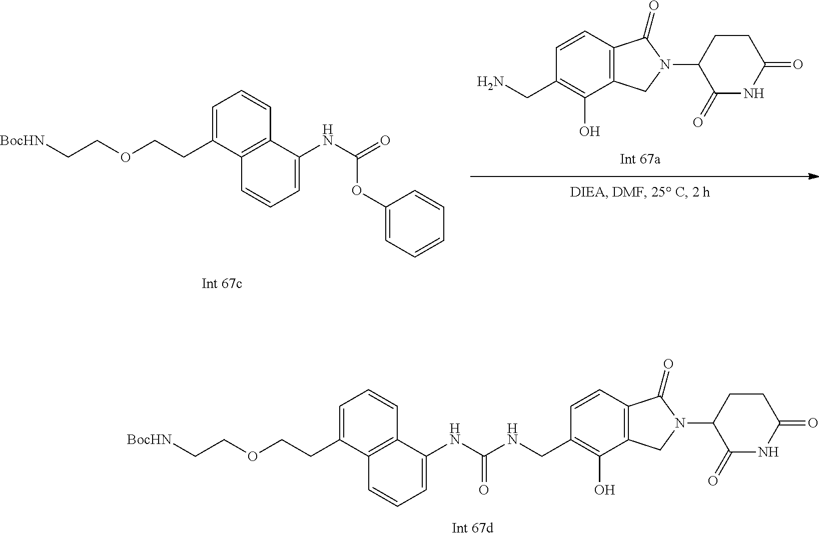 [SMILES: CC(C)(C)OC(=O)NCCOCCc1cccc2c(NC(=O)Oc3ccccc3)cccc12 ; NCc1ccc2c(c1O)CN(C1CCC(=O)NC1=O)C2=O ; CC(C)(C)OC(=O)NCCOCCc1cccc2c(NC(=O)NCc3ccc4c(c3O)CN(C3CCC(=O)NC3=O)C4=O)cccc12]

To a solution of tert-butyl (2-(2-(5-((phenoxycarbonyl)amino)naphthalen-1-yl)ethoxy)ethyl)carbamate Int 67c (150 mg, 333 μmol, 1.00 eq) and 3-(5-(aminomethyl)-4-hydroxy-1-oxoisoindolin-2-yl)piperidine-2,6-dione (125 mg, 433 μmol, 1.30 eq) in N,N-dimethylformamide (1.00 mL) was added N,N-diisopropylethylamine (129 mg, 999 μmol, 174 μL, 3.00 eq). The mixture was stirred at 25° C. for 2 h. The mixture was filtered. The filtrate was purified by reversed phase (0.1% formic acid condition) and lyophilized to afford tert-butyl (2-(2-(5-(3-((2-(2,6-dioxopiperidin-3-yl)-4-hydroxy-1-oxoisoindolin-5-yl)methyl)ureido)naphthalen-1-yl)ethoxy) ethyl)carbamate Int 67d (160 mg, 248 μmol, 74% yield) as a yellow solid. ¹H NMR (400 MHz, DMSO-d₆) δ=10.97 (s, 1H), 10.25 (br d, J=1.6 Hz, 1H), 8.75 (s, 1H), 7.95 (br d, J=8.0 Hz, 1H), 7.88 (d, J=7.6 Hz, 1H), 7.80 (br d, J=8.8 Hz, 1H), 7.50-7.40 (m, 4H), 7.22 (d, J=7.6 Hz, 1H), 7.16 (br t, J=5.6 Hz, 1H), 6.77-6.71 (m, 1H), 5.09 (dd, J=5.2, 13.2 Hz, 1H), 4.42-4.37 (m, 2H), 4.35-4.20 (m, 2H), 3.69 (br t, J=6.8 Hz, 2H), 3.43-3.40 (m, 3H), 3.10-3.05 (m, 2H), 2.95-2.87 (m, 1H), 2.60 (br d, J=18.0 Hz, 2H), 2.38 (br dd, J=4.0, 13.2 Hz, 1H), 2.05-1.98 (m, 1H), 1.37 (s, 9H).

### Step E. Preparation of Int 67e

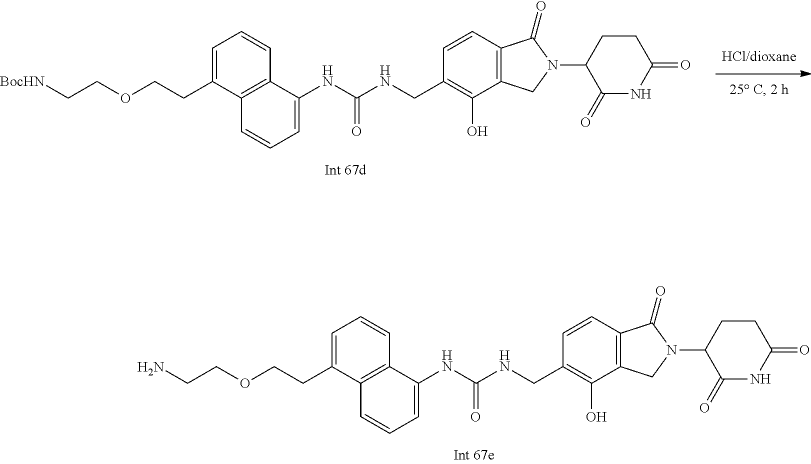 [SMILES: CC(C)(C)OC(=O)NCCOCCc1cccc2c(NC(=O)NCc3ccc4c(c3O)CN(C3CCC(=O)NC3=O)C4=O)cccc12 ; NCCOCCc1cccc2c(NC(=O)NCc3ccc4c(c3O)CN(C3CCC(=O)NC3=O)C4=O)cccc12]

A mixture of tert-butyl (2-(2-(5-(3-((2-(2,6-dioxopiperidin-3-yl)-4-hydroxy-1-oxoisoindolin-5-yl)methyl) ureido)naphthalen-1-yl)ethoxy)ethyl)carbamate Int 67d (80.0 mg, 124 μmol, 1.00 eq) in hydrochloric acid/dioxane (2.00 mL) was stirred at 25° C. for 2 h. The mixture was concentrated to afford a residue. The residue was purified by reversed phase (0.1% hydrochloric acid condition) and lyophilized to afford 1-(5-(2-(2-aminoethoxy)ethyl)naphthalen-1-yl)-3-((2-(2,6-dioxopiperidin-3-yl)-4-hydroxy-1-oxoisoindolin-5-yl) methyl)urea Int 67e (70.0 mg, 120 μmol, 97% yield) as a yellow solid. ¹H NMR (400 MHz, DMSO-d₆) δ=10.98 (s, 1H), 10.24 (s, 1H), 8.93 (s, 1H), 8.10-7.97 (m, 1H), 7.93-7.85 (m, 3H), 7.80 (d, J=8.4 Hz, 1H), 7.52-7.37 (m, 5H), 7.22 (d, J=7.6 Hz, 1H), 5.09 (dd, J=4.8, 13.2 Hz, 1H), 4.43-4.37 (m, 2H), 4.36-4.21 (m, 2H), 3.76 (t, J=7.2 Hz, 2H), 3.63 (t, J=5.4 Hz, 2H), 3.52-3.44 (m, 2H), 3.02-2.94 (m, 2H), 2.93-2.86 (m, 1H), 2.64-2.56 (m, 1H), 2.42-2.28 (m, 1H), 2.04-1.97 (m, 1H).

### Step F. Preparation of Compound IG-L-67

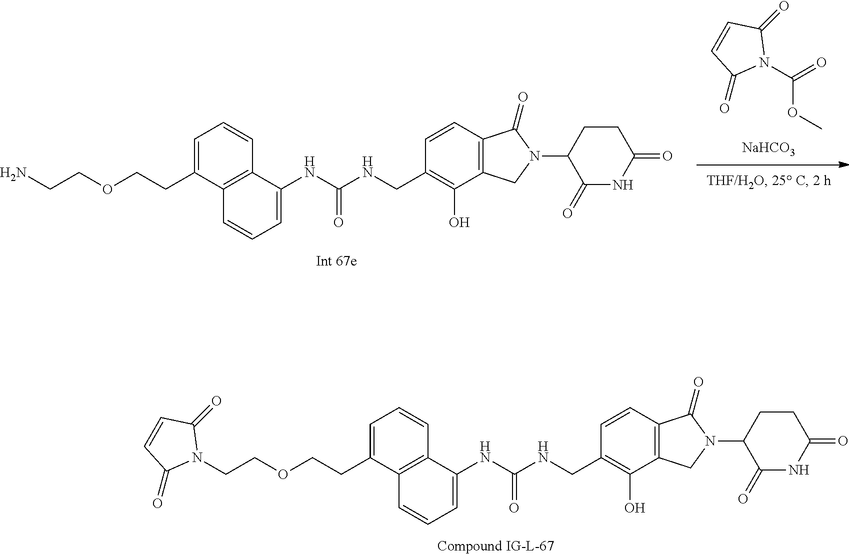

To a solution of 1-(5-(2-(2-aminoethoxy)ethyl)naphthalen-1-yl)-3-((2-(2,6-dioxopiperidin-3-yl)-4-hydroxy-1-oxoisoindolin-5-yl)methyl)urea Int 67e (70.0 mg, 120 gmol, 1.00 eq) in tetrahydrofuran (2.00 mL) and water (1.00 mL) was added a solution of methyl 2,5-dioxo-2,5-dihydro-1H-pyrrole-1-carboxylate (37.3 mg, 240 gmol, 37.3 μL, 2.00 eq) in tetrahydrofuran (2.00 mL). Then sodium bicarbonate (30.3 mg, 361 μmol, 14.0 μL, 3.00 eq) was added and it was stirred at 25° C. for 2 h. The mixture was quenched by water (30 mL), extracted with ethyl acetate (3×30 mL). The combined organic phases were washed with brine (2×30 mL), dried over anhydrous sodium sulfate, filtered and concentrated under reduced pressure to give a crude product. The crude product was purified by prep-HPLC (column: Phenomenex luna C18 150*25 mm*10 um; mobile phase: [water (formic acid)-acetonitrile]; gradient: 25%-55% B over 1 min), prep-HPLC (column: Phenomenex luna C18 150*25 mm*10 um; mobile phase: [water (hydrochloric acid)-acetonitrile]; gradient: 23%-53% B over 10 min), reversed phase (0.1% formic acid condition) and lyophilized to afford 1-(5-(2-(2-(2,5-dioxo-2,5-dihydro-TH-pyrrol-1-yl)ethoxy)ethyl)naphthalen-1-yl)-3-((2-(2,6-dioxopiperidin-3-yl)-4-hydroxy-1-oxoisoindolin-5-yl)methyl)urea Compound IG-L-67 (11.49 mg, 17.3 μmol, 14% yield, 94% purity, formate) as a white solid. ¹H NMR (400 MHz, DMSO-d₆) δ=10.96 (s, 1H), 10.23 (br s, 1H), 8.75 (s, 1H), 8.15 (s, 1H), 7.95 (d, J=8.8 Hz, 1H), 7.87 (d, J=7.6 Hz, 1H), 7.76 (d, J=8.4 Hz, 1H), 7.48-7.39 (m, 3H), 7.36-7.33 (m, 1H), 7.22 (d, J=7.6 Hz, 1H), 7.19-7.15 (m, 1H), 7.02-6.98 (m, 2H), 5.13-5.05 (m, 1H), 4.42-4.38 (m, 2H), 4.35-4.22 (m, 2H), 3.70-3.68 (m, 2H), 3.56 (br d, J=4.4 Hz, 4H), 3.21 (br d, J=7.2 Hz, 2H), 2.93 (br dd, J=1.6, 8.8 Hz, 1H), 2.63-2.63 (m, 1H), 2.40 (br d, J=4.4 Hz, 1H), 2.04-2.00 (m, 1H). MS (ESI) m/z 626.2 [M+H]⁺

The compounds in Table 21 were prepared in a manner similar to that described for Compound IG-L-67 using the appropriate compound as starting material.

TABLE 21

| IG-L | | |
|---|---|---|
| No. | Structure | ¹H NMR/MS |
| IG- L-66 | 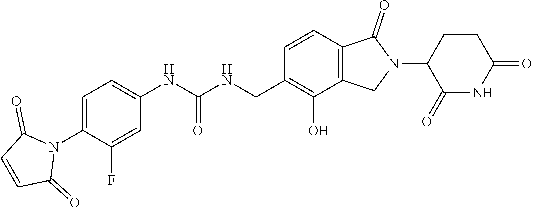 [SMILES: O=C1CCC(N2Cc3c(ccc(CNC(=O)Nc4ccc(N5C(=O)C=CC5=O)c(F)c4)c3O)C2=O)C(=O)N1] | ¹H NMR (400 MHz, DMSO-d₆) δ = 10.98 (br s, 1H), 10.09-9.85 (m, 1H), 9.15 (s, 1H), 7.61 (dd, J = 2.4, 12.8 Hz, 1H), 7.36 (d, J = 7.6 Hz, 1H), 7.30- 7.19 (m, 4H), 7.15 (dd, J = 2.0, 8.8 Hz, 1H), 6.96-6.80 (m, 1H), 5.10 (dd, J = 5.2, 13.2 Hz, 1H), 4.42-4.33 (m, 3H), 4.28-4.21 (m, 1H), 2.97-2.84 (m, 1H), 2.64-2.56 (m, 1H), 2.42-2.30 (m, 1H), 2.06-1.97 (m, 1H). LCMS (ESI) m/z 522.1 [M + H]⁺ |

### Example 101 Generation of Isoindolinone-Glutarimide Antibody Conjugates (IGAC) through Reduction of Native Disulfide Bonds of Non-Engineered Antibodies

A targeting moiety (e.g., a mAb at 3-8 mg/mL in PBS) is exchanged into HEPES (100 mM, pH 7.0, 1 mM DTPA) via molecular weight cut-off centrifugal filtration (Millipore, 30 kDa). The resultant solution is transferred to a tared 50 mL conical tube. The protein concentration is determined to be 3-8 mg/mL by A280. To the protein solution is added TCEP (2.0-10.0 equivalents, 1 mM stock) at room temperature and the resultant mixture is incubated at 37° C. for 30-90 minutes, with gentle shaking. Upon being cooled to room temperature, a stir bar is added to the reaction tube. Next, the compound of Structure (I) (5.0-10.0 equivalents, 10 mM DMSO) is added dropwise. The resultant reaction mixture is allowed to stir at ambient temperature for 30-60 minutes, at which point N-ethyl maleimide (3.0 equivalents, 100 mM DMA) is added. After an additional 15 minutes of stirring, N-acetylcysteine (6.0-11.0 equivalents, 50 mM HEPES) is added. The crude antibody conjugate is then exchanged into PBS and purified by preparative SEC (e.g. HiLoad 26/600, Superdex 200 μg) using PBS as the mobile phase. The pure fractions are concentrated via molecular weight cut-off centrifugal filtration (Millipore, 30 kDa), sterile filtered, and transferred to 15 mL conical tubes. The concentration was determined by UV-Vis and BCA, the monomer purity was determined by aSEC-HPLC and the final DAR was determined by HIC-HPLC and reduced LC-MS. The level of endotoxin determined by MCS analysis and free drug % was determined by RP-HPLC.

### Example 102 In Vitro Cell Proliferation Assay

One day prior to treatment with compounds or IGAC, cells were plated in white 96-well plates (SKBR: 3000 cells/well; CAL-51: 4000 cells/well). Compounds or ADCs were added in a serial-dilution manner reaching the indicated concentrations. Plates were incubated at 37 degrees for 96h (ADCs) or 72h (compounds) whereafter a 1:10 dilution of Cell Titer Glow was used to measure relative cell numbers after 10 min of incubation at room temperature.

On the day of treatment, 10,000 suspension cells/well were plated in white 96-well plates. Compounds or ADCs were added in a serial-dilution manner reaching the indicated concentrations. Plates were incubated at 37 degrees for 72 h whereafter a 1:10 dilution of Cell Titer Glow was used to measure relative cell numbers after 10 min of incubation at room temperature.

### Example 103 Degradation Assay Evaluated by Immunofluorescence Staining

One day prior to treatment with compounds, black 96 well plates were coated with fibronectin (0.2 μg/ml in PBS) for 45 min at room temperature and subsequently washed with PBS. CAL-51 or SKBR3 cells were seeded at 30K cells/well (1001) and incubated overnight at 37° C. whereafter compounds were added reaching indicated concentrations. 6 h after treatment the medium was removed, cells were washed with PBS and fixed with 10% formalin for 20 mins at room temperature. Following two PBS washes (150 μl), cells were permeabilized in 0.1% Triton X-100 in PBS for 8 mins at room temperature and subsequently washed again twice with PBS. Cells were blocked thereafter with 1% BSA in PBS for 45 mins. Primary antibody was diluted in blocking buffer and incubated with the cells overnight at 4° C. After three washes with PBS, cells were incubated with secondary antibody for 1 h at RT, washed and subsequently images were taken on an Operetta High-Content Imager.

For the determination of the DC50, a custom algorithm implemented in the PerkinElmer image analysis software Harmony-Acapella® was used.

### Example 104 Degradation Assay Evaluated with JESS (Western Blot)

One day prior to treatment cells were seeded in 6 or 12 well plates at a confluency of 70%. Compounds or IGAC were added in the indicated concentrations and cells incubated at 37° C. for indicated duration (6h, 16 h, 24h). After washing the cells with ice cold PBS, RIPA lysis buffer containing protease and phosphatase inhibitors was added and cells were scraped off the plates. After vigorous vortexing, lysates were incubated on ice for 10 min and subsequently centrifuged at 12000 rpm for 12 min at 4° C. Supernatants were transferred to a new tube and used for analysis on the JESS machine.

### Example 105 Treatment of HER2+ MDA-MB-453 Syngeneic Tumor Bearing NOD SCID Mice with an anti-Her2-Isoindolinone Glutarimide Conjugate

MDA-MB-453 cells were maintained in vitro in L-15 medium supplemented with 10% fetal bovine serum and 1% Antibiotic-Antimycotic at 37° C. in an atmosphere with 5% CO₂ in air. 5*10⁶ Cells resuspended in 0.2 mL of PBS mixed with Matrigel (50:50) were inoculated subcutaneously into the flank of female NOD SCID mice and allowed to grow to 150 mm³. Mice were dosed intravenously with the vehicle, isotype ADC control, or a representative compound of the disclosure (single IV injection, 3 and 10 mg/kg). Compound dosing solution was prepared on day of dosing by dilution in its formulation buffer. Tumor volumes were measured twice weekly over a period of 28 days.

### Example 106 Treatment of CD22+ WSU-DLCL2 Syngeneic Tumor Bearing CB17 SCID Mice with Anti-CD22-Isoindolinone Glutarimide Conjugates

The objective of the study was to evaluate the in vivo anti-tumor efficacy of pinatuzumab, IGAC-59 and IGAC-62 in the WSU-DLCL2 lymphoma xenograft model grown in female CB17 SCID mice

The WSU-DLCL2 tumor cells were maintained in vitro in RPMI-1640 medium supplemented with 10% FBS and 1% antibiotic-antimycotic at 37° C. in an atmosphere with 5% CO₂ in air. WSU-DLCL2 tumor cells are a diffuse large B-cell lymphoma (DLBCL) cell line, expressing the CD22 antigen, which is a protein found on the surface of B cells. CD22 is a well-validated tumor target for the treatment of B-cell leukemia and lymphoma. The tumor cells were routinely sub-cultured twice weekly. The cells growing in exponential growth phase were harvested and counted for tumor inoculation. The mice were inoculated subcutaneously in the right flank with WSU-DLCL2 tumor cells (10*10⁶) in 0.1 ml of PBS and allowed to grow to 200 mms. The mice were grouped (n=9 mice per group) and treatments were started on day 21 after cell inoculation when the average tumor volume reached 198 mm³. Mice were dosed intravenously with the vehicle, pinatuzumab, or a representative compound of the disclosure, including IGAC-59 and IGAC-62 (single IV injection, 3, 10 and 20 mg/kg). Compound dosing solution was prepared on day of dosing by dilution in its formulation buffer (20 mM histidine, 7% sucrose, pH 5.5). Tumor size was measured twice weekly over a period of 28 days in two dimensions using a caliper, and the volume was expressed in mm3 using the formula: V=0.5 a×b² where a and b are the long and short diameters of the tumor, respectively. Body weight was measured twice a week and animals would be euthanized if they lost over 20% of body weight from the weight at day 1. Tumor growth inhibition (TGI) was calculated for each group using the formula: TGI (%)=[1−(Ti−T0)/(Vi−V0)]×100; Ti is the average tumor volume of a treatment group on D21 post dosing, T0 is the average tumor volume of the treatment group on the day of treatment start, Vi is the average tumor volume of the vehicle control group on the same day with Ti, and V0 is the average tumor volume of the vehicle group on the day of treatment start.

The anti-tumor efficacy of pinatuzumab, IGAC-59 and IGAC-62 on WSU-DLCL2 xenografts grown in female CB17 SCID mice was evaluated. The tumor growth inhibition curves are shown in FIG. 1. Tumor growth inhibition analysis is provided in Table 22.

FIG. 1 shows a graph of in vivo tumor volume over time in the treatment of CD22+ WSU-DLCL2 syngeneic tumor bearing CB17 SCID mice with pinatuzumab, and anti-CD22 conjugates IGAC-59 and IGAC-62 (Table 3).

TABLE 22
Tumor Growth Inhibition Analysis: The inhibition rates
of pinatuzumab, IGAC-59 and IGAC-62 on WSU-DLCL2 tumors
are calculated with tumor size data on D21 post dosing.

| | | Tumor Size | TGI | |
|---|---|---|---|---|
| Group | Treatment | (mm³) | (%) | p value |
| 1 | Vehicle, IV, Single dose | 1639 | — | — |
| 2 | Pinatuzumab, 20 mg/kg, | 1601 | 2.58 | p > 0.05 |
| | IV, Single dose | | | |
| 4 | IGAC-59, 3 mg/kg, IV, | 616 | 71.03 | p < 0.001 |
| | Single dose | | | |
| 5 | IGAC-59, 10 mg/kg, IV, | 341 | 90.03 | p < 0.001 |
| | Single dose | | | |
| 6 | IGAC-59, 20 mg/kg, IV, | 60 | 109.56 | p < 0.001 |
| | Single dose | | | |
| 7 | IGAC-62, 20 mg/kg, IV, | 8 | 113.21 | p < 0.001 |
| | Single dose | | | |

Naked antibody pinatuzumab at 20 mg/kg did not show any activity in WSU-DLCL2 tumors, whereas conjugate IGAC-59 and IGAC-62 (Table 3) treated groups showed significant tumor growth delay compared to vehicle group. At the end of the study, IGAC-59 20 mg/kg and IGAC-62 20 mg/kg treated groups reached tumor regression demonstrating the efficacy of antibody conjugate compositions in treating cancer.

Mean body weight remained within +/−5% of initial body weight (BW) for all groups. Food supplement was given to 3 mice across groups 5 and 7 due to a BW loss greater than 10%. Mice treated with conjugates IGAC-59 and IGAC-62 recovered and/or stabilized their body weight upon addition of food supplement demonstrating the safety of antibody conjugate compositions.

### Example 107 Treatment of CD22+ WSU-DLCL2 Syngeneic Tumor Bearing CB17 SCID Mice with Anti-CD22-Isoindolinone Glutarimide Conjugates

The objective of the study was to evaluate the in vivo anti-tumor efficacy of Pinatuzumab, IGAC-53, IGAC-60 and IGAC-62 in the WSU-DLCL2 lymphoma xenograft model grown in female CB17 SCID mice

The WSU-DLCL2 tumor cells were maintained in vitro in RPMI-1640 medium supplemented with 10% FBS and 1% antibiotic-antimycotic at 37° C. in an atmosphere with 5% CO₂ in air. The tumor cells were routinely sub-cultured twice weekly. The cells growing in exponential growth phase were harvested and counted for tumor inoculation. The mice were inoculated subcutaneously in the right flank with WSU-DLCL2 tumor cells (10*10⁶) in 0.1 ml of PBS and allowed to grow to 200 mm³. The mice were grouped (n=9 mice per group) and treatments were started on day 21 after cell inoculation when the average tumor volume reached 186 mm³. Mice were dosed intravenously with the vehicle, pinatuzumab, or a representative compound of the disclosure (single IV injection, 3, 10 and 20 mg/kg). Compound dosing solution was prepared on day of dosing by dilution in its formulation buffer (20 mM histidine, 7% sucrose, pH 5.5). Tumor size was measured twice weekly over a period of 28 days in two dimensions using a caliper, and the volume was expressed in mm³ using the formula: V=0.5 a×b² where a and b are the long and short diameters of the tumor, respectively. Body weight was measured twice a week and animals would be euthanized if they lost over 20% of body weight from the weight at day 1. Tumor growth inhibition (TGI) was calculated for each group using the formula: TGI (%)=[1−(Ti−T0)/(Vi−V0)]×100; Ti is the average tumor volume of a treatment group on D24 post dosing, T0 is the average tumor volume of the treatment group on the day of treatment start, Vi is the average tumor volume of the vehicle control group on the same day with Ti, and V0 is the average tumor volume of the vehicle group on the day of treatment start.

In this study, the anti-tumor efficacy of Pinatuzumab, IGAC-53, IGAC-60 and IGAC-62 on WSU-DLCL2 xenografts grown in female CB17 SCID mice was evaluated. The body weight change curves and tumor growth curves are shown in FIG. 2, tumor growth inhibition data is shown in Table 23.

FIG. 2 shows a graph of in vivo tumor volume over time in the treatment of CD22+ WSU-DLCL2 syngeneic tumor bearing CB17 SCID mice with pinatuzumab, and anti-CD22 conjugates IGAC-53, IGAC-60 and IGAC-62 (Table 3).

TABLE 23
Tumor Growth Inhibition Analysis: The inhibition
rates of pinatuzumab, IGAC-53 and IGAC-60 and
IGAC-62 on WSU-DLCL2 tumors are calculated
with tumor size data on D24 post dosing.

| | | Tumor Size | TGI | |
|---|---|---|---|---|
| Group | Treatment | (mm³) | (%) | p value |
| 1 | Vehicle | 1817 | — | — |
| 2 | IGAC-62 5 mg/kg | 510 | 80.21 | p < 0.001 |
| 3 | IGAC-62 20 mg/kg | 53 | 108.11 | p < 0.001 |
| 4 | IGAC-53 5 mg/kg | 728 | 66.72 | p < 0.001 |
| 5 | IGAC-53 20 mg/kg | 354 | 89.73 | p < 0.001 |
| 6 | IGAC-60 5 mg/kg | 860 | 58.65 | p < 0.001 |
| 7 | IGAC-60 20 mg/kg | 457 | 83.41 | p < 0.001 |
| 9 | pinatuzumab 5 mg/kg | 1713 | 6.35 | p > 0.05 |

Naked antibody pinatuzumab at 5 mg/kg did not show any activity in WSU-DLCL2 tumors. In contrast, all ADC treated groups showed significant tumor growth delay compared to that of vehicle and pinatuzumab and in a dose dependent manner. At the end of the study, IGAC-62 20 mg/kg treated group reached tumor stasis. Mean body weight remained within +/−5% of initial BW for all groups. Food supplement was given to 7 mice across group 2, 4, 5, 7, 8 due to a BW loss greater than 10%. Mice recovered and/or stabilized their body weight upon addition of food supplement. Overall, the tested DACs were well tolerated.

### Example 108 Treatment of HER2+ NCI-N87 Gastric Tumor Xenograft Bearing Female BALB/c Nude Mice with Anti-HER2-Isoindolinone Glutarimide Conjugates

The objective of the study was to evaluate the in vivo anti-tumor efficacy of IGAC-58 and IGAC-61 in NCI-N87 gastric tumor xenograft model grown in female BALB/c nude mice.

The NCI-N87 tumor cells were maintained in vitro in RPMI-1640 medium supplemented with 10% fetal bovine serum and 1% antibiotic-antimycotic at 37° C. in an atmosphere with 5% CO₂ in air. NCI-N87 is a HER2-positive adenocarcinoma, gastric cancer xenograft model utilized in studying pre-clinical monotherapies and combination therapies. The tumor cells were routinely sub-cultured twice weekly. The cells growing in exponential growth phase were harvested and counted for tumor inoculation. The mice were inoculated subcutaneously in the right flank with NCI-N87 tumor cells (10*10⁶) in 0.2 mL of PBS mixed with Matrigel (50:50). The treatments were started on day 5 after cell inoculation when the average tumor volume reached 200 mm³. Mice were dosed intravenously with the vehicle, isotype ADC control, or a representative compound of the disclosure (single IV injection, 5 and 20 mg/kg). Compound dosing solution was prepared on day of dosing by dilution in its formulation buffer (20 mM histidine, 7% sucrose, pH 5.5). The main efficacy endpoint was to see impact of compounds on tumor growth. Tumor size was measured twice weekly over a period of 28 days in two dimensions using a caliper, and the volume was expressed in mm³ using the formula: V=0.5 a×b² where a and b are the long and short diameters of the tumor, respectively. Body weight was measured twice a week and animals would be euthanized if they lost over 20% of body weight from the weight at day 1.

FIG. 3 shows a graph of in vivo tumor volume over time in the treatment of HER2+ NCI-N87 gastric tumor xenograft bearing female BALB/c nude mice with anti-HER2 conjugates IGAC-58 and IGAC-61 (Table 3).

Results summary and discussion: IGAC-61 showed a dose dependent response at 5 vs 20 mg/kg. At the 5 mg/kg dose IGAC-61 led to regression over the first 10 days post dosing, then showed tumor regrowth from D14. At the 20 mg/kg doses, both IGAC-61 and IGAC-58 led to regression over the first 10 days, and tumor regression was maintained throughout the 28 days post dosing. (FIG. 3), demonstrating efficacy in treating HER2 positive cancer.

In this study, all treatments were well-tolerated and mice slightly gained body weight over the course of the study. No clinically relevant body weight loss or other clinical signs due to the treatment were observed, demonstrating the safety of the anti-HER2 conjugates.

### Example 109 Treatment of HER2+ NCI-N87 Gastric Tumor Xenograft Bearing Female BALB/c Nude Mice with Anti-HER2-Isoindolinone Glutarimide Conjugates

The objective of the study was to evaluate the in vivo anti-tumor efficacy of IGAC-54, IGAC-55, IGAC-56 and IGAC-57 in NCI-N87 gastric tumor xenograft model grown in female BALB/c nude mice.

The NCI-N87 tumor cells were maintained in vitro in RPMI-1640 medium supplemented with 10% fetal bovine serum and 1% antibiotic-antimycotic at 37° C. in an atmosphere with 5% CO₂ in air. The tumor cells were routinely sub-cultured twice weekly. The cells growing in exponential growth phase were harvested and counted for tumor inoculation. The mice were inoculated subcutaneously in the right flank with NCI-N87 tumor cells (10*10⁶) in 0.2 mL of PBS mixed with Matrigel (50:50). The treatments were started on day 5 after cell inoculation when the average tumor volume reached 200 mm³. Mice were dosed intravenously with the vehicle, isotype ADC control, or a representative compound of the disclosure (single IV injection, 3 and 10 mg/kg). Compound dosing solution was prepared on day of dosing by dilution in its formulation buffer (20 mM histidine, 7% sucrose, pH 5.5). The main efficacy endpoint was to see impact of compounds on tumor growth. Tumor size was measured twice weekly over a period of 21 days in two dimensions using a caliper, and the volume was expressed in mm³ using the formula: V=0.5 a×b² where a and b are the long and short diameters of the tumor, respectively. Body weight was measured twice a week and animals would be euthanized if they lost over 20% of body weight from the weight at day 1.

FIG. 4 shows a graph of in vivo tumor volume over time in the treatment of HER2+ NCI-N87 gastric tumor xenograft bearing female BALB/c nude mice with anti-HER2 conjugates IGAC-54, IGAC-55, IGAC-56 and IGAC-57 (Table 3).

Results summary and discussion: IGAC-57 showed a dose dependent response at 3 vs 10 mg/kg. At the 3 mg/kg dose IGAC-57 and IGAC-56 at the 10 mg/kg dose led to regression over the first 14 days post dosing, then showed tumor stasis at D21. At the 10 mg/kg doses, IGAC-57, IGAC-54 and IGAC-55 led to regression over the first 10 days, and tumor regression was maintained throughout the 21 days post dosing (FIG. 4).

In this study, all treatments were well-tolerated and mice slightly gained body weight over the course of the study. No clinically relevant body weight loss or other clinical signs due to the treatment were observed, demonstrating safety of the anti-HER2 conjugates.

All references, including publications, patent applications, and patents, cited herein are hereby incorporated by reference to the same extent as if each reference were individually and specifically indicated to be incorporated by reference and were set forth in its entirety herein.

1. An antibody conjugate composition comprising an isoindolinone-glutarimide moiety covalently attached to an antibody by an antibody linker, wherein the antibody binds to a tumor-associated antigen or cell-surface receptor.

2. The antibody conjugate composition of claim 1 having Formula I: 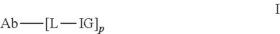
or a pharmaceutically acceptable salt thereof,
wherein:
Ab is the antibody;
L is the antibody linker, wherein L has the formula: 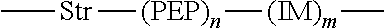
Str is a stretcher unit covalently attached to the antibody, wherein Str is selected from the structure: 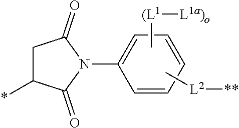
L¹ is selected from a bond, C₁-C₁₂ alkyldiyl, and C₁-C₄₀ heteroalkyldiyl;
L^{1a} is selected from an amino acid, amino, hydroxyl, halide, hydrogen, carboxylic acid, glycerol, or a sugar such as pentaerythritol, maltitol, sorbitol, xylitol, erythritol, isomalt, or combinations thereof;
o is 0, 1, or 2;
L² is selected from a bond, C₁-C₁₂ alkyldiyl, and C₁-C₄₀ heteroalkyldiyl;
* indicates the point of attachment to a cysteine thiol of Ab; and
** indicates the point of attachment to PEP or to the isoindolinone-glutarimide moiety;
PEP is a protease-cleavable, peptide unit covalently attached to Str and IM or IG;
IM is an immolative unit covalently attached to IG;
n is 0 or 1; and m is 0 or 1;
IG is the isoindolinone-glutarimide moiety selected from Formulae Ia and Ib: 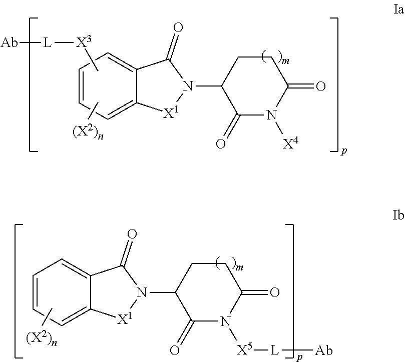
or a pharmaceutically acceptable salt, stereoisomer, mixture of stereoisomers, tautomer, or isotopic analog thereof, wherein:
m is 1;
X¹ is CH₂;
X² is independently selected from the group consisting of F, Cl, Br, I, —CN, C₁-C₁₂ alkyl, C₂-C₁₂ alkenyl, C₂-C₁₂ alkynyl, C₁-C₁₂ heteroalkyl, —(C₁-C₁₂ heteroalkyldiyl)-(C₆-C₂₀ aryldiyl), —(C₁-C₁₂ heteroalkyldiyl)-(C₆-C₂₀ aryldiyl)-(C₁-C₁₂ heteroalkyl), —(C₁-C₁₂ heteroalkyldiyl)-(C₆-C₂₀ aryl), —(C₁-C₁₂ heteroalkyldiyl)-(C₆-C₂₀ aryldiyl)-(C₁-C₁₂ heteroalkyl), (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, —OR^{a}, —OC(═O)R^{a}, —SR^{a}, —S(O)R^{a}, —S(O)₂R^{a}, —S(O)₂NR^{a}, and —S(O)₃H;
R^{a} is independently selected from H, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
R^{b} is independently selected from H, —OH, —O—(C₁-C₆ alkyl), C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
n is 0, 1, 2, 3, or 4;
X³ is selected from the group consisting of a bond, O, C₁-C₁₂ alkyldiyl, C₂-C₁₂ alkenyldiyl, C₂-C₁₂ alkynyldiyl, C₁-C₁₂ heteroalkyldiyl, —(C₁-C₆ alkyldiyl)-(C₆-C₂₀ aryldiyl)-, —(C₁-C₆ alkyldiyl)NR^{a}—, —(C₁-C₆ alkyldiyl)O—, —(C₁-C₆ alkyldiyl)-(C₃-C₂₀ carbocyclyldiyl)-, —(C₁-C₆ alkyldiyl)-(C₂-C₂₀ heterocyclyldiyl)-, —(C₁-C₆ alkyldiyl)-(C₁-C₂₀ heteroaryldiyl)-, —(C₁-C₁₂ heteroalkyldiyl)-(C₆-C₂₀ aryldiyl)-, —(C₁-C₁₂ heteroalkyldiyl)-(C₆-C₂₀ aryldiyl)-(C₁-C₁₂ heteroalkyldiyl)-, —(C₁-C₁₂ heteroalkyldiyl)-(C₆-C₂₀ aryldiyl)-(C₁-C₁₂ heteroalkyldiyl)-(C₆-C₂₀ aryldiyl)-(C₁-C₁₂ heteroalkyldiyl)-, —(C₁-C₁₂ heteroalkyldiyl)-(C₆-C₂₀ aryl)-, C₆-C₂₀ aryldiyl, C₃-C₂₀ carbocyclyldiyl, C₂-C₂₀ heterocyclyldiyl, and C₁-C₂₀ heteroaryldiyl;
X⁴ is H;
X⁵ is selected from the group consisting of a bond, C₁-C₁₂ alkyldiyl, C₂-C₁₂ alkenyldiyl, C₂-C₁₂ alkynyldiyl, C₁-C₁₂ heteroalkyldiyl, —(C₁-C₆ alkyldiyl)-(C₆-C₂₀ aryldiyl), —(C₁-C₆ alkyldiyl)-(C₆-C₂₀ aryldiyl)-O—(C₂-C₂₀ heterocyclyl), —(C₁-C₆ alkyldiyl)NR^{a}—, —(C₁-C₆ alkyldiyl)O—, —(C₁-C₆ alkyldiyl)-(C₃-C₂₀ carbocyclyldiyl), —C₁-C₆ alkyldiyl)-(C₂-C₂₀ heterocyclyldiyl), —(C₁-C₆ alkyldiyl)-(C₁-C₂₀ heteroaryldiyl), —(C₁-C₁₂ heteroalkyldiyl)-(C₆-C₂₀ aryldiyl), —(C₁-C₁₂ heteroalkyldiyl)-(C₆-C₂₀ aryldiyl)-(C₁-C₁₂ heteroalkyldiyl), C₆-C₂₀ aryldiyl, C₃-C₂₀ carbocyclyldiyl, C₂-C₂₀ heterocyclyldiyl, and C₁-C₂₀ heteroaryldiyl; and
p is an integer from 1 to 12;
wherein each alkyl, alkyldiyl, alkenyl, alkenyldiyl, alkynyl, alkynyldiyl, heteroalkyl, heteroalkyldiyl, aryl, aryldiyl, carbocyclyl, carbocyclyldiyl, heterocyclyl, heterocyclyldiyl, heteroaryl, and heteroaryldiyl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

3. The antibody conjugate composition of claim 2 wherein a phenolic oxygen of the isoindolinone-glutarimide moiety is attached to the antibody linker.

4. The antibody conjugate composition of claim 2 wherein the nitrogen of the glutarimide group of the isoindolinone-glutarimide moiety is attached to the antibody linker.

5. The antibody conjugate composition of claim 2 wherein a solubilizing unit selected from polyglycine, polysarcosine, polyethyleneoxy (PEG), C₁-C₄₀ heteroalkyl, and a glycoside, or combinations thereof is attached to: (1) a phenolic oxygen, or (2) the nitrogen of the glutarimide group of the isoindolinone-glutarimide moiety.

6. The antibody conjugate composition of claim 5 wherein the terminus of the solubilizing unit is a group selected from an amino acid, amino, hydroxyl, hydrogen, carboxylic acid, glycerol, or a sugar such as pentaerythritol, maltitol, sorbitol, xylitol, erythritol, isomalt, or combinations thereof.

7. (canceled)

8. The antibody conjugate composition of claim 1, wherein the antibody linker is covalently attached to a cysteine amino acid of the antibody.

9-15. (canceled)

16. The antibody conjugate composition of claim 2 wherein X² comprises a —NHC(═O)NH— group.

17. The antibody conjugate composition of claim 2 wherein X² is selected from the group consisting of C₁-C₁₂ heteroalkyl, —(C₁-C₁₂ heteroalkyldiyl)-(C₆-C₂₀ aryl), and —(C₁-C₁₂ heteroalkyldiyl)-(C₆-C₂₀ aryldiyl)-(C₁-C₁₂ heteroalkyl).

18. The antibody conjugate composition of claim 17 wherein X² is —(C₁-C₁₂ heteroalkyldiyl)-(C₆-C₂₀ aryl).

19. The antibody conjugate composition of claim 18 wherein X² is —CH₂NHC(═O)NH—(C₆-C₂₀ aryl).

20. The antibody conjugate composition of claim 18 wherein X² is: 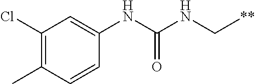
wherein:
** indicates the point of attachment to the isoindolinone-glutarimide moiety.

21. The antibody conjugate composition of claim 2 wherein X³ is O.

22. The antibody conjugate composition of claim 2 wherein X³ comprises a —NHC(═O)NH— group.

23. The antibody conjugate composition of claim 2 wherein X³ is —(C₁-C₁₂ heteroalkyldiyl)-(C₆-C₂₀ aryldiyl)-(C₁-C₁₂ heteroalkyldiyl).

24. The antibody conjugate composition of claim 23 wherein X³ is —CH₂NHC(═O)NH—(C₆-C₂₀ aryldiyl)-(C₁-C₁₂ heteroalkyldiyl).

25. The antibody conjugate composition of claim 2 wherein X³ of Formula Ia is selected from —(C₁-C₁₂ heteroalkyldiyl)-(C₆-C₂₀ aryldiyl)-O—, —(C₁-C₁₂ heteroalkyldiyl)-(C₆-C₂₀ aryldiyl)-N(R^{a})—, and —(C₁-C₁₂ heteroalkyldiyl)-(C₆-C₂₀ aryldiyl)-(C₁-C₁₂ heteroalkyldiyl).

26. The antibody conjugate composition of claim 25 wherein X³ is —CH₂CH₂CH₂NHC(═O)NH—(C₆-C₂₀ aryldiyl)-O—.

27. The antibody conjugate composition of claim 25 wherein X³ is selected from the structures: 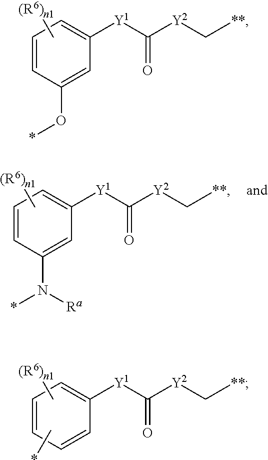
wherein:
* indicates the point of attachment to L;
** indicates the point of attachment to the phenyl glutarimide moiety;
R⁶ is independently selected from F, Cl, Br, I, —CN, OH, —O—(C₁-C₁₂ alkyl), C₁-C₁₂ alkyl, C₂-C₁₂ alkenyl, C₂-C₁₂ alkynyl, C₁-C₁₂ heteroalkyl, C₆-C₂₀ aryl, C₃-C₂₀ carbocyclyl, C₂-C₂₀ heterocyclyl, and C₁-C₂₀ heteroaryl;
n1 is 1, 2, 3, or 4;
Y¹ is selected from CF₂ and NH; and
Y² is selected from NH, O, and CH₂.

28. The antibody conjugate composition of claim 2 wherein X³ is selected from: 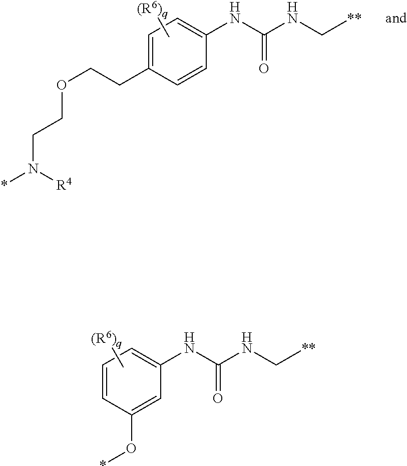
wherein:
* indicates the point of attachment to L;
** indicates the point of attachment to the isoindolinone-glutarimide moiety;
R⁴ is selected from H and C₁-C₁₂ alkyl;
R⁶ is independently selected from F, Cl, Br, I, —CN, OH, C₁-C₁₂ alkyl, C₂-C₁₂ alkenyl, C₂-C₁₂ alkynyl, and C₁-C₁₂ heteroalkyl; and
q is selected from 0, 1, 2, 3, and 4.

29. The antibody conjugate composition of claim 2 wherein X³ is —(C₁-C₁₂ heteroalkyldiyl)-(C₆-C₂₀ aryl).

30. The antibody conjugate composition of claim 29 wherein X³ is selected from: 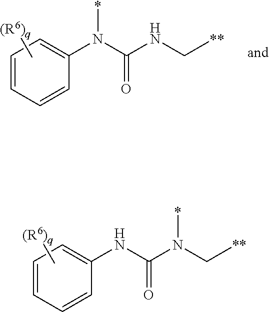
wherein:
* indicates the point of attachment to L;
** indicates the point of attachment to the isoindolinone-glutarimide moiety;
R⁶ is independently selected from F, Cl, Br, I, —CN, OH, C₁-C₁₂ alkyl, C₂-C₁₂ alkenyl, C₂-C₁₂ alkynyl, and C₁-C₁₂ heteroalkyl; and
q is selected from 0, 1, 2, 3, and 4.

31. The antibody conjugate composition of claim 2 of Formula Ia:

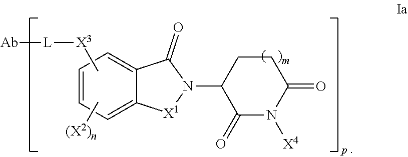

32. The antibody conjugate composition of claim 31 wherein X² is —OH and nis 1.

33. The antibody conjugate composition of claim 31 selected from the structures:

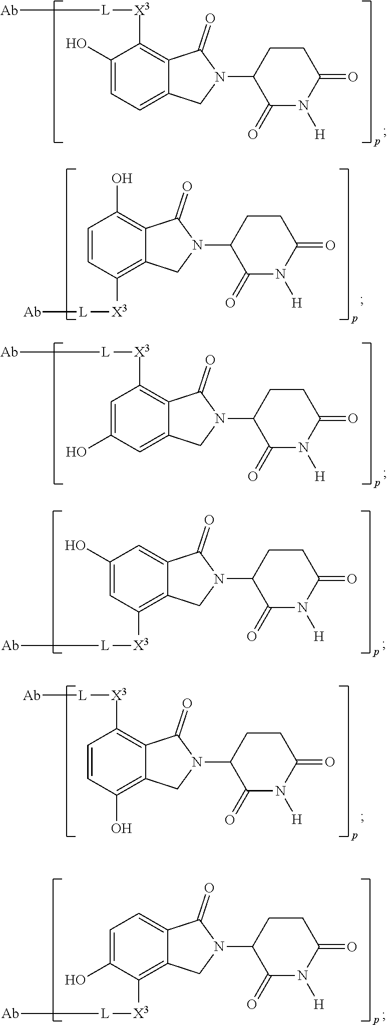

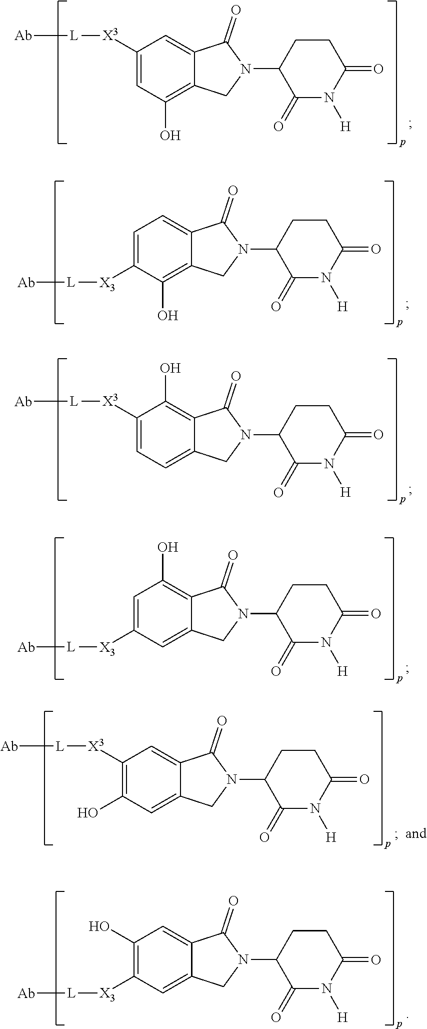

34-37. (canceled)

38. The antibody conjugate composition of claim 2 of Formula Ib:

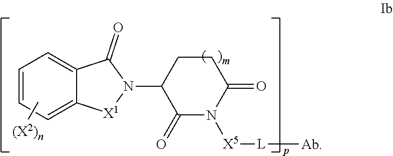

39. The antibody conjugate composition of claim 38 wherein n is 2, 3 or 4 and one of X² is —OH.

40. The antibody conjugate composition of claim 38 selected from the structures:

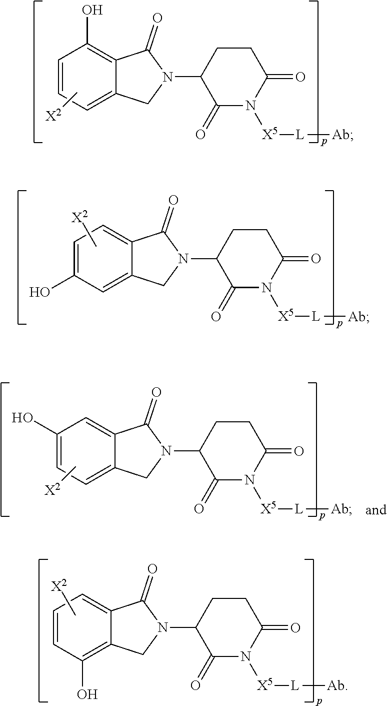

41. The antibody conjugate composition of claim 38 of Formula Ib wherein X⁵ is selected from —(C₁-C₁₂ heteroalkyldiyl)-(C₆-C₂₀ aryldiyl) and —(C₁-C₆ alkyldiyl)-(C₆-C₂₀ aryldiyl).

42. The antibody conjugate composition of claim 38 selected from the Formulae Ic-g: 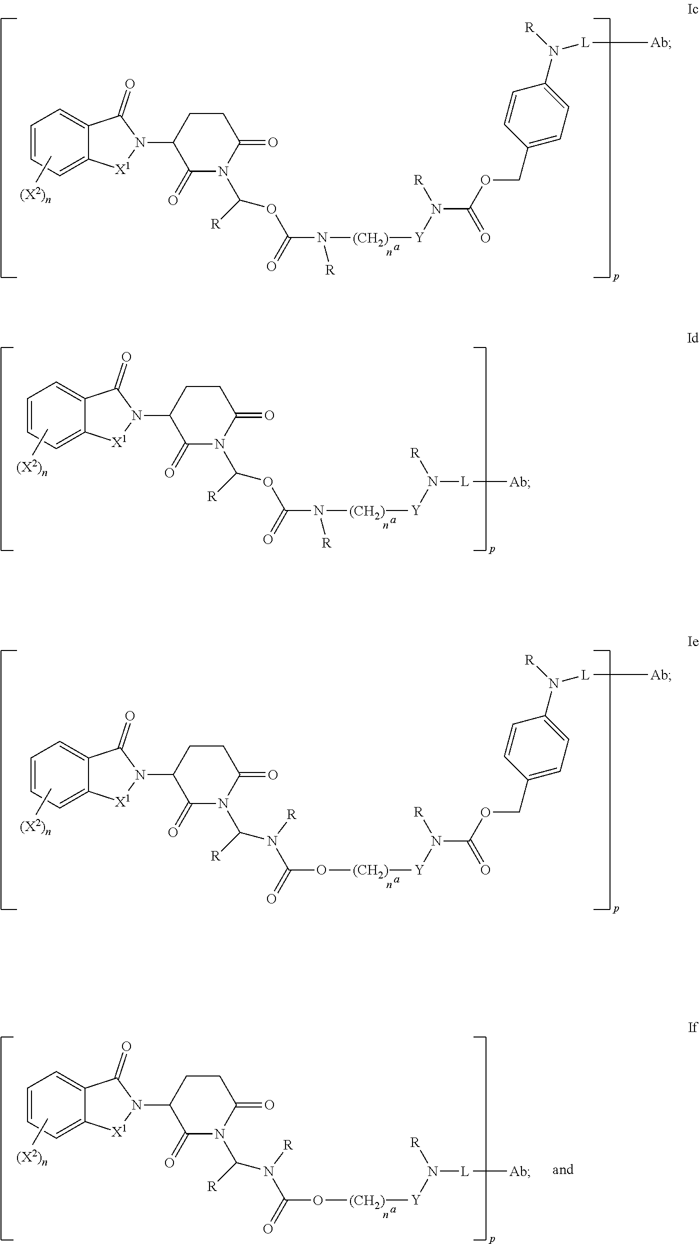 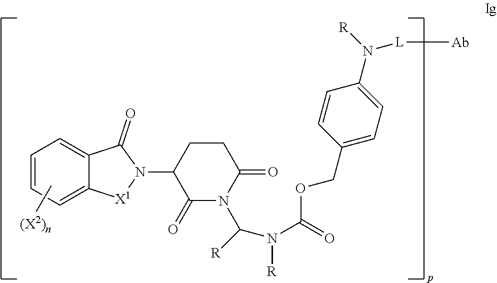
where R is independently selected from H, C₁-C₆ alkyl, and O—(C₁-C₆ alkyl); n^{a} is an integer from 1 to 5; and Y is CH₂ or O.

43. (canceled)

44. (canceled)

45. The antibody conjugate composition of claim 38 selected from the Formulae Ih and Ii: 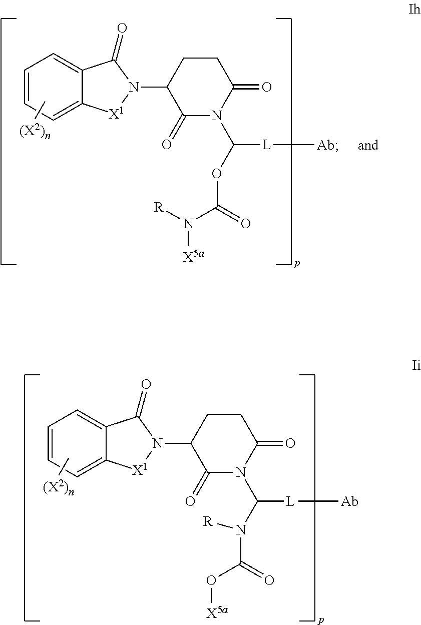
wherein R is selected from H, C₁-C₆ alkyl, and O—(C₁-C₆ alkyl); and X^{5a} is —(C₁-C₆ alkyldiyl)-(C₆-C₂₀ aryldiyl)-O—(C₂-C₂₀ heterocyclyl).

46. The antibody conjugate composition of claim 45 wherein C₂-C₂₀ heterocyclyl of X^{5a} is a glucuronide.

47. The antibody conjugate composition of claim 46 wherein X^{5a} is: 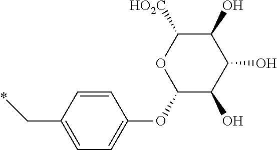 [SMILES: *Cc1ccc(O[C@@H]2O[C@H](C(=O)O)[C@@H](O)[C@H](O)[C@H]2O)cc1]
wherein * indicates the point of attachment.

48. (canceled)

49. The antibody conjugate composition of claim 45 selected from the Formulae Ij-m: 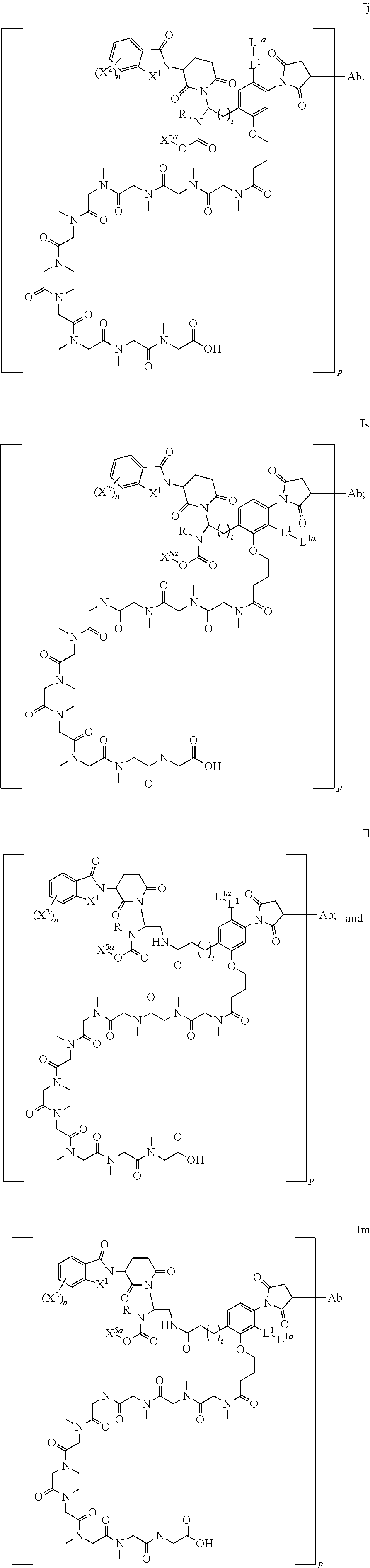
where t is selected from 0, 1, 2, 3, and 4.

50. The antibody conjugate composition of claim 49 wherein L¹ is a bond and L^{1a} is F.

51. The antibody conjugate composition of claim 2 wherein the antibody linker comprises an immolating group.

52. The antibody conjugate composition of claim 2 wherein the antibody linker comprises a peptide unit.

53. (canceled)

54. The antibody conjugate composition of claim 2 wherein IG is attached to L by Str.

55. The antibody conjugate composition of claim 2 wherein IG is attached to L by PEP.

56. The antibody conjugate composition of claim 2 wherein IG is attached to L by IM.

57. The antibody conjugate composition of claim 2 wherein L is a branched linker and Str is covalently attached to: (i) the antibody; and (ii) a solubilizing unit comprising a group selected from polyglycine, polysarcosine, polyethyleneoxy (PEG), and a glycoside, or combinations thereof, wherein the terminus of the solubilizing unit is a group selected from an amino acid, amino, hydroxyl, hydrogen, carboxylic acid, glycerol, or a sugar such as pentaerythritol, maltitol, sorbitol, xylitol, erythritol, isomalt, or combinations thereof.

58. The antibody conjugate composition of claim 57 wherein the solubilizing unit and the terminus of the solubilizing unit covalently attached to Str are selected from the structures: 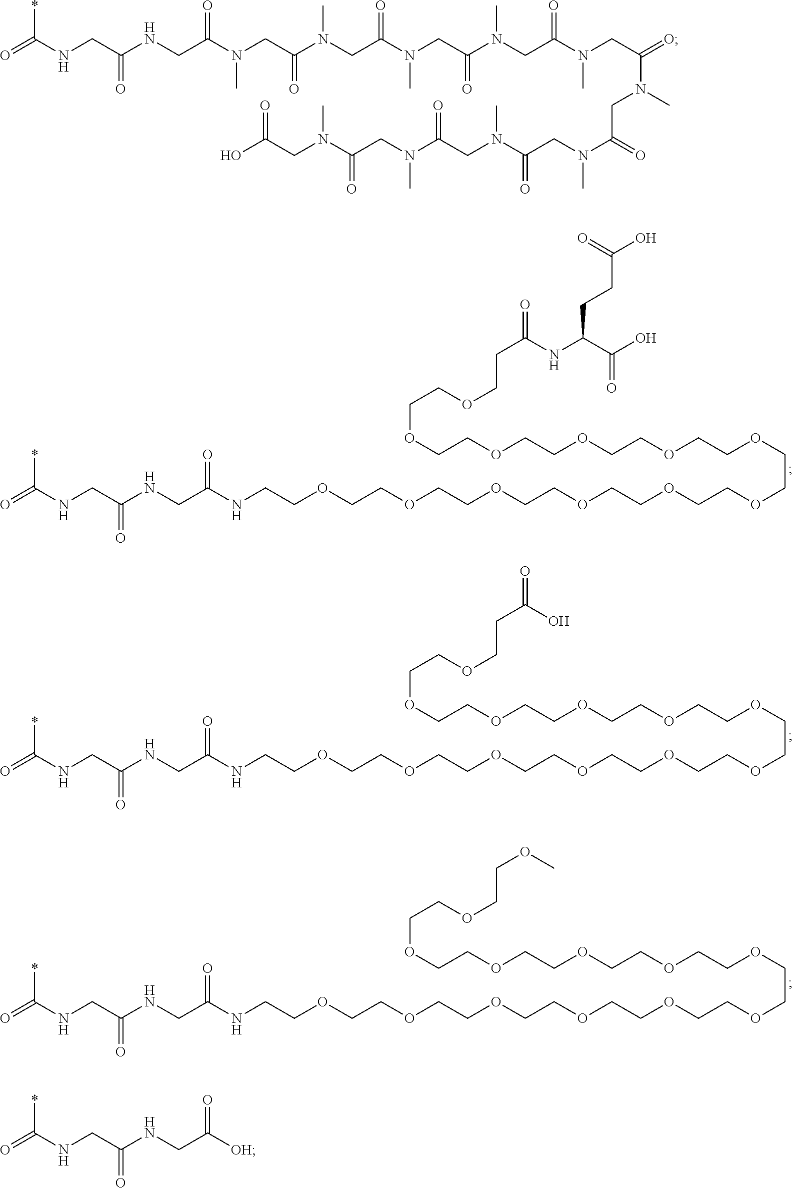 [SMILES: *C(=O)NCC(=O)NCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O ; *C(=O)NCC(=O)NCC(=O)NCCOCCOCCOCCOCCOCCOCCOCCOCCOCCOCCOCCOCCC(=O)N[C@@H](CCC(=O)O)C(=O)O ; *C(=O)NCC(=O)NCC(=O)NCCOCCOCCOCCOCCOCCOCCOCCOCCOCCOCCOCCOCCC(=O)O ; *C(=O)NCC(=O)NCC(=O)NCCOCCOCCOCCOCCOCCOCCOCCOCCOCCOCCOCCOCCOC ; *C(=O)NCC(=O)NCC(=O)O] 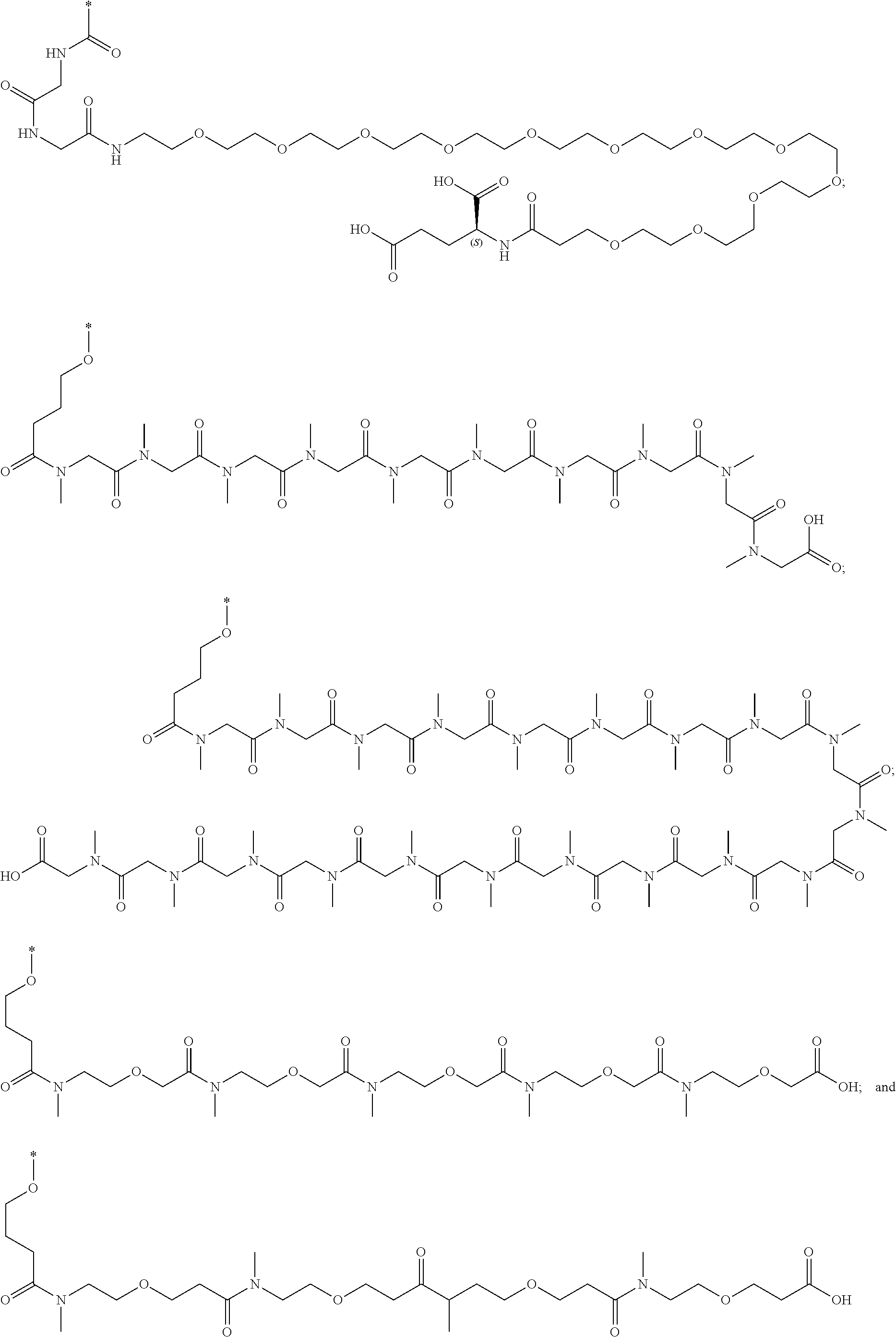
wherein * indicates the point of attachment to Str.

59. The antibody conjugate composition of claim 57 wherein L is a branched linker and PEP is covalently attached to: (i) Str and IM or IG; and (ii) a solubilizing unit comprises a group selected from polyglycine, polysarcosine, polyethyleneoxy (PEG), and a glycoside, or combinations thereof, wherein the terminus of the solubilizing unit is a group selected from an amino acid, amino, hydroxyl, hydrogen, carboxylic acid, glycerol, or a sugar such as pentaerythritol, maltitol, sorbitol, xylitol, erythritol, isomalt, or combinations thereof.

60. The antibody conjugate composition of claim 57 wherein L is a branched linker and IM is covalently attached to: (i) IG; and (ii) a solubilizing unit comprises a group selected from polyglycine, polysarcosine, polyethyleneoxy (PEG), and a glycoside, or combinations thereof, wherein the terminus of the solubilizing unit is a group selected from an amino acid, amino, hydroxyl, hydrogen, carboxylic acid, glycerol, or a sugar such as pentaerythritol, maltitol, sorbitol, xylitol, erythritol, isomalt, or combinations thereof.

61-65. (canceled)

66. The antibody conjugate composition of claim 2 wherein L¹ or L² comprise a solubilizing unit selected from polyglycine, polysarcosine, polyethyleneoxy (PEG), and a glycoside, or combinations thereof.

67. The antibody conjugate composition of claim 66 wherein L¹ or L² comprises glucuronic acid having the structure:

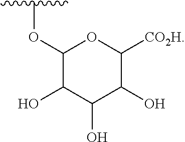 [SMILES: *OC1OC(C(=O)O)C(O)C(O)C1O]

68. The antibody conjugate composition of claim 2 wherein L¹ or L² is selected from (N(CH₃)CH₂C(═O))_{q}, (N(CH₃)CH₂CH₂C(═O))_{q}, N(CH₃)CH₂CH₂OCH₂CH₂C(═O))_{q}, (CH₂CH₂O)_{q}, (CH₂CH₂O)_{q}C(═O), and (CH₂CH₂O)_{q}—CH₂, where q is an integer from 2 to 20.

69. (canceled)

70. The antibody conjugate composition of claim 2 wherein n is 1, m is 1, and PEP-IM has the formula: 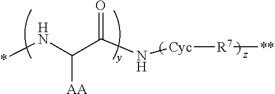
wherein * indicates the point of attachment to Str and ** indicates the point of attachment to IG;
AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and glucuronic acid having the structure: 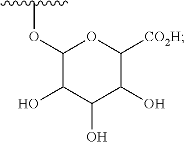 [SMILES: *OC1OC(C(=O)O)C(O)C(O)C1O]
R⁷ is selected from the group consisting of —CH(R⁸)O—, —CH₂—, —CH₂N(R⁸)CH(R⁸)—, —CH(R⁸)OC(═O)—, —CH(R⁸)OC(═O)N(R⁸)CH(R⁸)—, —CH(R⁸)OP(═O)₂₀CH(R⁸)—, and —CH(R⁸)OC(═O)N(R⁸)—(C₁-C₆ alkyldiyl)-N(R⁸)C(═O)OCH(R⁸)—;
R⁸ is selected from H, C₁-C₆ alkyl, C(═O)—C₁-C₆ alkyl, and —C(═O)N(R⁹)₂;
R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12; and
z is 0 or 1.

71. (canceled)

72. The antibody conjugate composition of claim 70 wherein AA is independently selected from H, —CH₃, —CH(CH₃)₂, —CH₂(C₆H₅), —CH₂C(O)NH₂, —CH₂CH₂CO2H, —CH₂CO2H, —CH₂CH₂CH₂CH₂NH₂, —CH₂CH₂CH₂NHC(NH)NH₂, —CH₂CH(CH₃)₂, —CH₂SO₃H, and —CH₂CH₂CH₂NHC(O)NH₂; or two AA form a 5-membered ring proline amino acid.

73. The antibody conjugate composition of claim 70 wherein PEP-IM comprises at least one natural or unnatural amino acid side chain AA substituted with C₁-C₄₀ heteroalkyl.

74. The antibody conjugate composition of claim 73 wherein C₁-C₄₀ heteroalkyl comprises a solubilizing unit selected from polyglycine, polysarcosine, polyethyleneoxy (PEG), and a glycoside, or combinations thereof.

75. The antibody conjugate composition of claim 74 wherein the solubilizing unit is selected from (N(CH₃)CH₂C(═O))_{q}, (N(CH₃)CH₂CH₂C(═O))_{q}, N(CH₃)CH₂CH₂OCH₂CH₂C(═O))_{q}, (CH₂CH₂O)q, (CH₂CH₂O)_{q}C(═O), and (CH₂CH₂O)_{q}—CH₂, where q is an integer from 2 to 20.

76. The antibody conjugate composition of claim 70 wherein y is 2, PEP is a dipeptide, and PEP-IM has the formula: 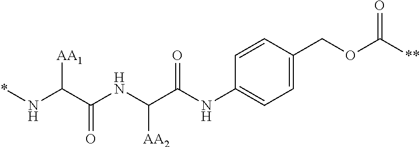
wherein AA₁ and AA₂ are independently selected from a side chain of a naturally-occurring amino acid.

77. The antibody conjugate composition of claim 76 wherein the dipeptide is selected from ala-ala, val-cit, and phe-ala.

78. The antibody conjugate composition of claim 76 wherein AA₁ is —CH(CH₃)₂, and AA₂ is —CH₂CH₂CH₂NHC(O)NH₂.

79. The antibody conjugate composition of claim 76 wherein AA₁ and AA₂ are each —CH₃.

80. The antibody conjugate composition of claim 70 wherein y is 3, PEP is a tripeptide, and PEP-IM has the formula:

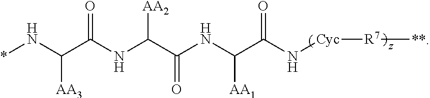

81. The antibody conjugate composition of claim 70 wherein y is 4, PEP is a tetrapeptide PEP-IM, and has the formula:

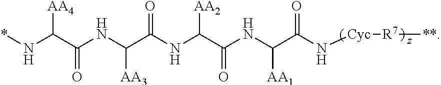

82. The antibody conjugate composition of claim 70 where z is 1 and IM has the formula: 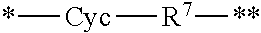
wherein:
* indicates the point of attachment to PEP; and
** indicates the point of attachment to IG.

83. The antibody conjugate composition of claim 82 where R⁷ has the formula: 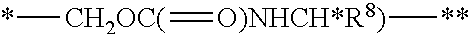
wherein:
R⁸ is selected from H and C₁-C₆ alkyl;
* indicates the point of attachment to PEP; and
** indicates the point of attachment to IG.

84. The antibody conjugate composition of claim 83 where R⁸ is H.

85. The antibody conjugate composition of claim 83 where R⁸ is —CH₃.

86. The antibody conjugate composition of claim 82 wherein IM is selected from the formulae: 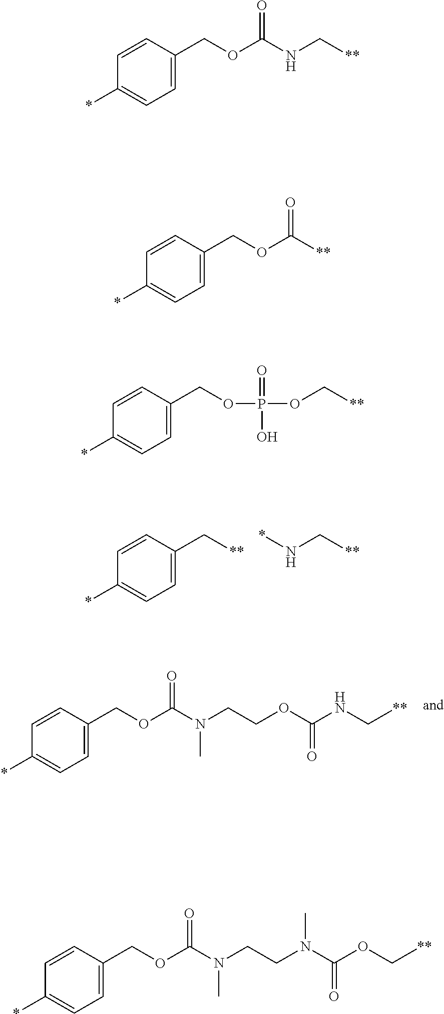
wherein:
* indicates the point of attachment to PEP; and
** indicates the point of attachment to IG.

87. The antibody conjugate composition of claim 82 where IM is selected from the formulae: 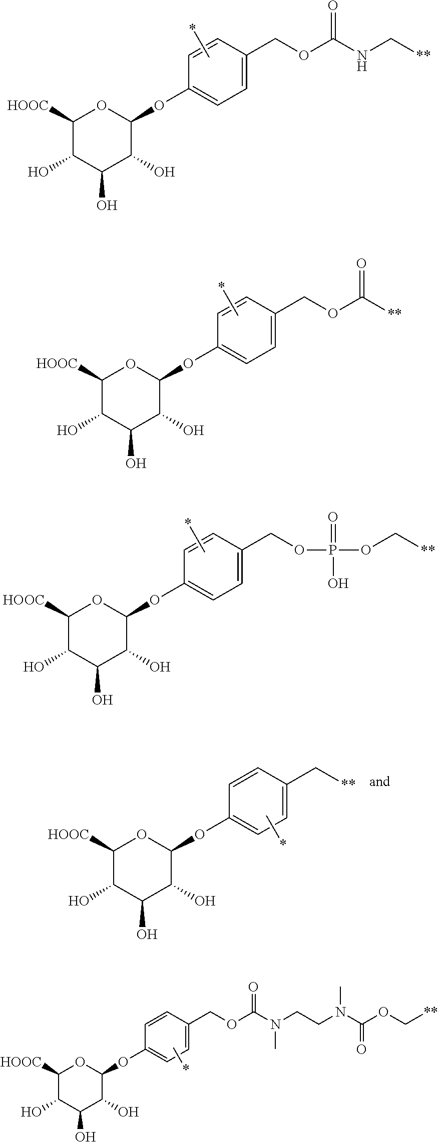
wherein:
* indicates the point of attachment to PEP; and
** indicates the point of attachment to IG.

88. An isoindolinone-glutarimide linker compound selected from Formulae IIa and IIb: 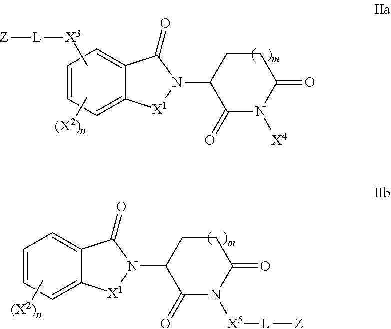
or a pharmaceutically acceptable salt, stereoisomer, mixture of stereoisomers, tautomer, or isotopic analog thereof, wherein:
m is 1;
X¹ is CH₂;
X² is independently selected from the group consisting of F, Cl, Br, I, —CN, C₁-C₁₂ alkyl, C₂-C₁₂ alkenyl, C₂-C₁₂ alkynyl, C₁-C₁₂ heteroalkyl, —(C₁-C₁₂ heteroalkyldiyl)-(C₆-C₂₀ aryldiyl), —(C₁-C₁₂ heteroalkyldiyl)-(C₆-C₂₀ aryldiyl)-(C₁-C₁₂ heteroalkyl), —(C₁-C₁₂ heteroalkyldiyl)-(C₆-C₂₀ aryl), —(C₁-C₁₂ heteroalkyldiyl)-(C₆-C₂₀ aryldiyl)-(C₁-C₁₂ heteroalkyl), (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, —OR^{a}, —OC(═O)R^{a}, —SR^{a}, —S(O)R^{a}, —S(O)₂R^{a}, —S(O)₂NR^{a}, and —S(O)₃H;
R^{a} is independently selected from H, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
R^{b} is independently selected from H, OH, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
n is 0, 1, 2, 3, or 4;
X³ is selected from the group consisting of a bond, O, C₁-C₁₂ alkyldiyl, C₂-C₁₂ alkenyldiyl, C₂-C₁₂ alkynyldiyl, C₁-C₁₂ heteroalkyldiyl, —(C₁-C₆ alkyldiyl)-(C₆-C₂₀ aryldiyl)-, —(C₁-C₆ alkyldiyl)NR^{a}—, —(C₁-C₆ alkyldiyl)O—, —(C₁-C₆ alkyldiyl)-(C₃-C₂₀ carbocyclyldiyl)-, —(C₁-C₆ alkyldiyl)-(C₂-C₂₀ heterocyclyldiyl)-, —(C₁-C₆ alkyldiyl)-(C₁-C₂₀ heteroaryldiyl)-, —(C₁-C₁₂ heteroalkyldiyl)-(C₆-C₂₀ aryldiyl)-, —(C₁-C₁₂ heteroalkyldiyl)-(C₆-C₂₀ aryldiyl)-(C₁-C₁₂ heteroalkyldiyl)-, —(C₁-C₁₂ heteroalkyldiyl)-(C₆-C₂₀ aryldiyl)-(C₁-C₁₂ heteroalkyldiyl)-(C₆-C₂₀ aryldiyl)-(C₁-C₁₂ heteroalkyldiyl)-, —(C₁-C₁₂ heteroalkyldiyl)-(C₆-C₂₀ aryl)-, C₆-C₂₀ aryldiyl, C₃-C₂₀ carbocyclyldiyl, C₂-C₂₀ heterocyclyldiyl, and C₁-C₂₀ heteroaryldiyl;
X⁴ is H;
X⁵ is selected from the group consisting of a bond, C₁-C₁₂ alkyldiyl, C₂-C₁₂ alkenyldiyl, C₂-C₁₂ alkynyldiyl, C₁-C₁₂ heteroalkyldiyl, —(C₁-C₆ alkyldiyl)-(C₆-C₂₀ aryldiyl), —(C₁-C₆ alkyldiyl)-(C₆-C₂₀ aryldiyl)-O—(C₂-C₂₀ heterocyclyl), —(C₁-C₆ alkyldiyl)NR^{a}—, —(C₁-C₆ alkyldiyl)O—, —(C₁-C₆ alkyldiyl)-(C₃-C₂₀ carbocyclyldiyl), —C₁-C₆ alkyldiyl)-(C₂-C₂₀ heterocyclyldiyl), —(C₁-C₆ alkyldiyl)-(C₁-C₂₀ heteroaryldiyl), —(C₁-C₁₂ heteroalkyldiyl)-(C₆-C₂₀ aryldiyl), —(C₁-C₁₂ heteroalkyldiyl)-(C₆-C₂₀ aryldiyl)-(C₁-C₁₂ heteroalkyldiyl), C₆-C₂₀ aryldiyl, C₃-C₂₀ carbocyclyldiyl, C₂-C₂₀ heterocyclyldiyl, and C₁-C₂₀ heteroaryldiyl;
L is the antibody linker, wherein L has the formula: 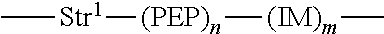
Str¹ is a stretcher unit covalently attached to Z, wherein Z-Str¹ has the structure: 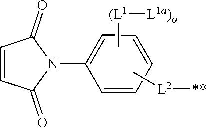
L¹ is selected from a bond, C₁-C₁₂ alkyldiyl, and C₁-C₄₀ heteroalkyldiyl:
L^{1a} is selected from an amino acid, amino, hydroxyl, halide, hydrogen, carboxylic acid, glycerol, or a sugar such as pentaerythritol, maltitol, sorbitol, xylitol, erythritol, isomalt, or combinations thereof;
o is 0, 1, or 2;
L² is selected from a bond, C₁-C₁₂ alkyldiyl, and C₁-C₄₀ heteroalkyldiyl; and
** indicates the point of attachment to PEP or to the isoindolinone-glutarimide moiety;
PEP is a protease-cleavable, peptide unit covalently attached to Str¹ and IM;
IM is an immolative unit covalently attached the isoindolinone-glutarimide moiety; and
n is 0 or 1; and m is 0 or 1; and
wherein each alkyl, alkyldiyl, alkenyl, alkenyldiyl, alkynyl, alkynyldiyl, heteroalkyl, heteroalkyldiyl, aryl, aryldiyl, carbocyclyl, carbocyclyldiyl, heterocyclyl, heterocyclyldiyl, heteroaryl, and heteroaryldiyl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

89-95. (canceled)

96. The isoindolinone-glutarimide linker compound of claim 88 wherein L¹ or L² comprise a solubilizing unit selected from polyglycine, polysarcosine, polyethyleneoxy (PEG), and a glycoside, or combinations thereof.

97. The isoindolinone-glutarimide linker compound of claim 88 wherein L¹ or L² is selected from (N(CH₃)CH₂C(═O))_{q}, (N(CH₃)CH₂CH₂C(═O))_{q}, N(CH₃)CH₂CH₂OCH₂CH₂C(═O))_{q}, (CH₂CH₂O)_{q}, (CH₂CH₂O)_{q}C(═O), and (CH₂CH₂O)_{q}—CH₂, where q is an integer from 2 to 20.

98. An isoindolinone-glutarimide compound selected from Formula III: 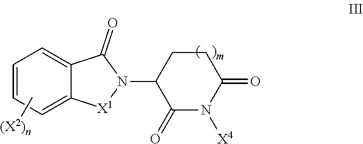
or a pharmaceutically acceptable salt, stereoisomer, mixture of stereoisomers, tautomer, or isotopic analog thereof, wherein:
m is 1;
X¹ is CH₂;
X² is independently selected from the group consisting of F, Cl, Br, I, —CN, C₁-C₁₂ alkyl, C₂-C₁₂ alkenyl, C₂-C₁₂ alkynyl, C₁-C₁₂ heteroalkyl, —(C₁-C₁₂ heteroalkyldiyl)-(C₆-C₂₀ aryldiyl), —(C₁-C₁₂ heteroalkyldiyl)-(C₆-C₂₀ aryldiyl)-(C₁-C₁₂ heteroalkyl), —(C₁-C₁₂ heteroalkyldiyl)-(C₆-C₂₀ aryl), —(C₁-C₁₂ heteroalkyldiyl)-(C₆-C₂₀ aryldiyl)-(C₁-C₁₂ heteroalkyl), (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, —OR^{a}, —OC(═O)R^{a}, —SR^{a}, —S(O)R^{a}, —S(O)₂R^{a}, —S(O)₂NR^{a}, and —S(O)₃H;
R^{a} is independently selected from H, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
R^{b} is independently selected from H, OH, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
n is 0, 1, 2, 3, or 4; and
X⁴ is H;
wherein each alkyl, alkyldiyl, alkenyl, alkenyldiyl, alkynyl, alkynyldiyl, heteroalkyl, heteroalkyldiyl, aryl, aryldiyl, carbocyclyl, carbocyclyldiyl, heterocyclyl, heterocyclyldiyl, heteroaryl, and heteroaryldiyl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

99-104. (canceled)

105. The isoindolinone-glutarimide compound of claim 98 wherein X² comprises a —NHC(═O)NH— group.

106. The isoindolinone-glutarimide compound of claim 98 wherein X² is selected from the group consisting of C₁-C₁₂ heteroalkyl, —(C₁-C₁₂ heteroalkyldiyl)-(C₆-C₂₀ aryl), and —(C₁-C₁₂ heteroalkyldiyl)-(C₆-C₂₀ aryldiyl)-(C₁-C₁₂ heteroalkyl).

107. The isoindolinone-glutarimide compound of claim 98 wherein X² is —(C₁-C₁₂ heteroalkyldiyl)-(C₆-C₂₀ aryl).

108. The isoindolinone-glutarimide compound of claim 107 wherein X² is —CH₂NHC(═O)NH—(C₆-C₂₀ aryl).

109. The isoindolinone-glutarimide compound of claim 106 wherein X² is: 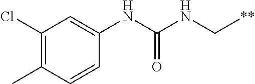
where ** indicates the point of attachment.

110-113. (canceled)

114. The isoindolinone-glutarimide compound of claim 98 selected from:

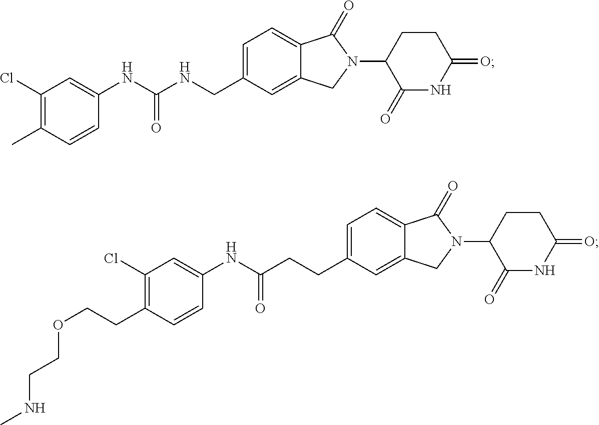 [SMILES: Cc1ccc(NC(=O)NCc2ccc3c(c2)CN(C2CCC(=O)NC2=O)C3=O)cc1Cl ; CNCCOCCc1ccc(NC(=O)CCc2ccc3c(c2)CN(C2CCC(=O)NC2=O)C3=O)cc1Cl]

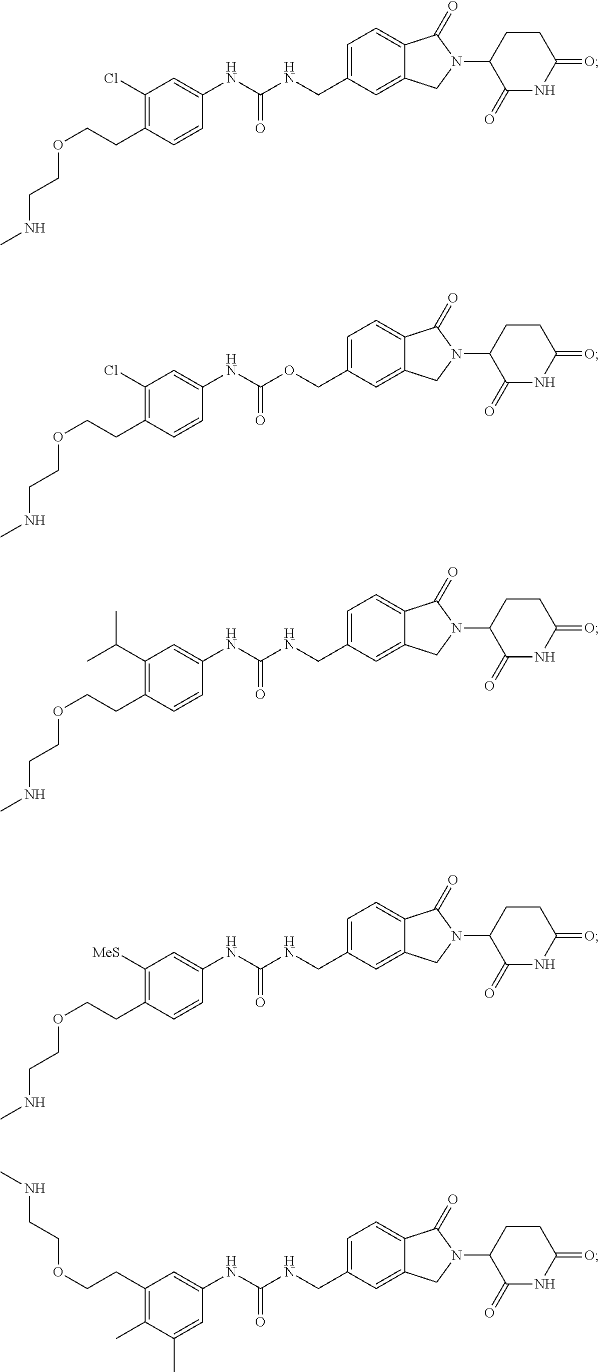 [SMILES: CNCCOCCc1ccc(NC(=O)NCc2ccc3c(c2)CN(C2CCC(=O)NC2=O)C3=O)cc1Cl ; CNCCOCCc1ccc(NC(=O)OCc2ccc3c(c2)CN(C2CCC(=O)NC2=O)C3=O)cc1Cl ; CNCCOCCc1ccc(NC(=O)NCc2ccc3c(c2)CN(C2CCC(=O)NC2=O)C3=O)cc1C(C)C ; CNCCOCCc1ccc(NC(=O)NCc2ccc3c(c2)CN(C2CCC(=O)NC2=O)C3=O)cc1SC ; CNCCOCCc1cc(NC(=O)NCc2ccc3c(c2)CN(C2CCC(=O)NC2=O)C3=O)cc(C)c1C]

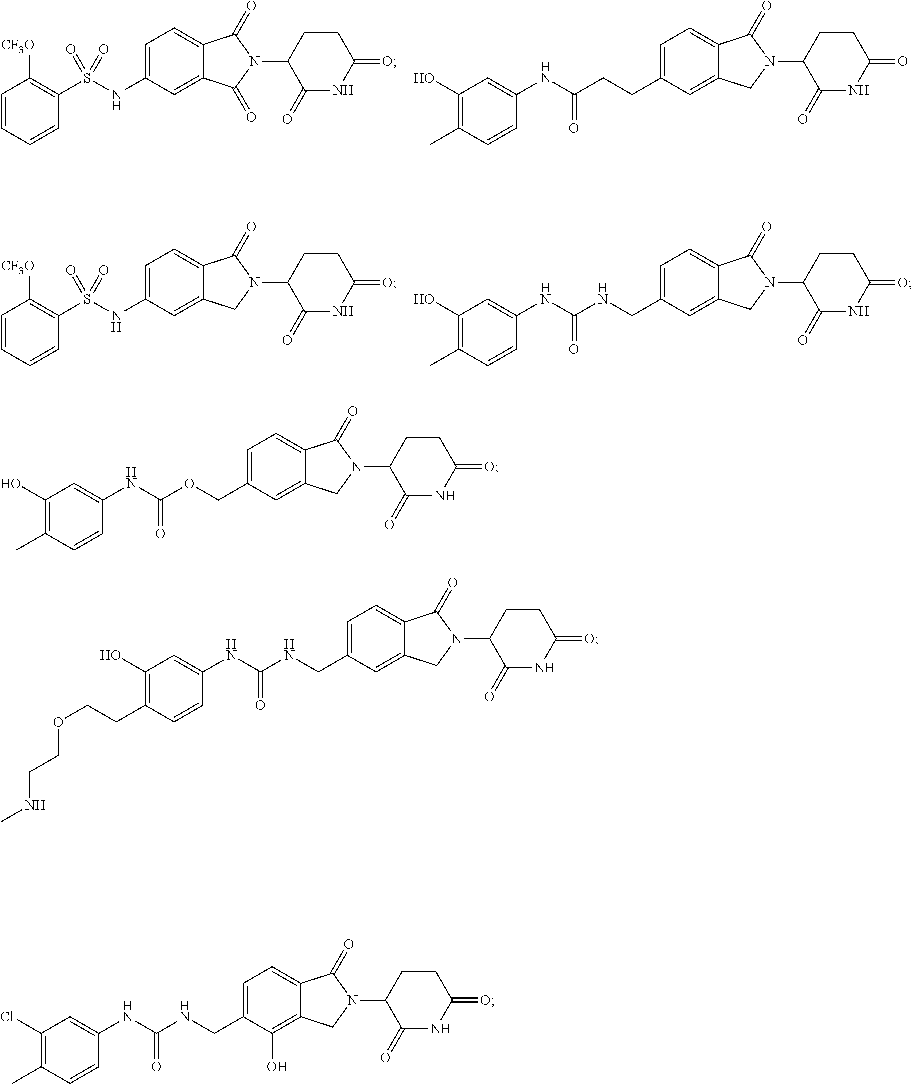

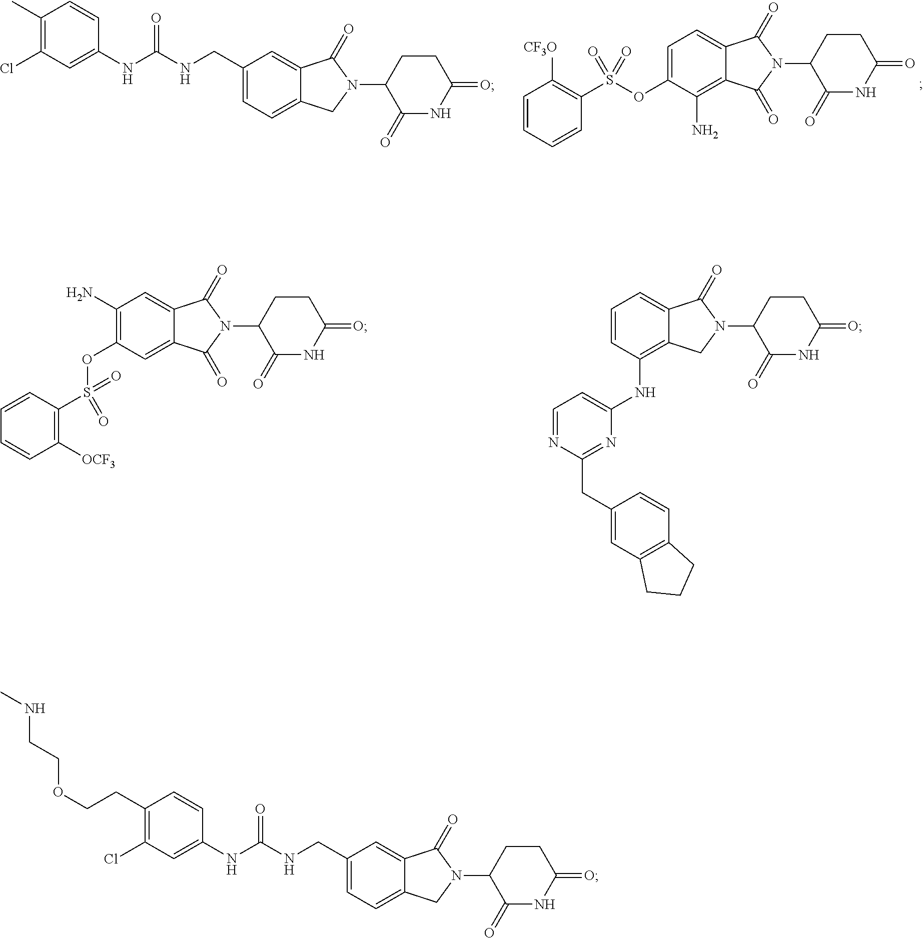

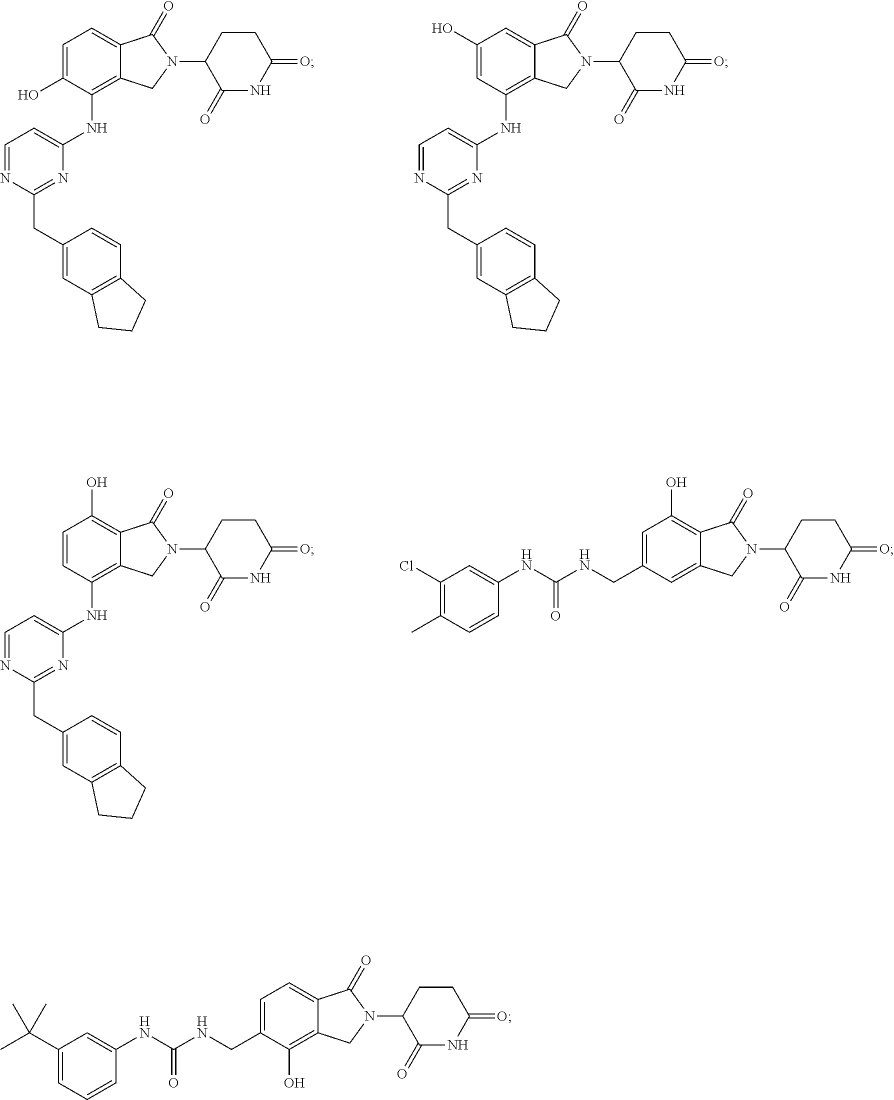 [SMILES: O=C1CCC(N2Cc3c(ccc(O)c3Nc3ccnc(Cc4ccc5c(c4)CCC5)n3)C2=O)C(=O)N1 ; O=C1CCC(N2Cc3c(Nc4ccnc(Cc5ccc6c(c5)CCC6)n4)cc(O)cc3C2=O)C(=O)N1 ; O=C1CCC(N2Cc3c(Nc4ccnc(Cc5ccc6c(c5)CCC6)n4)ccc(O)c3C2=O)C(=O)N1 ; Cc1ccc(NC(=O)NCc2cc(O)c3c(c2)CN(C2CCC(=O)NC2=O)C3=O)cc1Cl ; CC(C)(C)c1cccc(NC(=O)NCc2ccc3c(c2O)CN(C2CCC(=O)NC2=O)C3=O)c1]

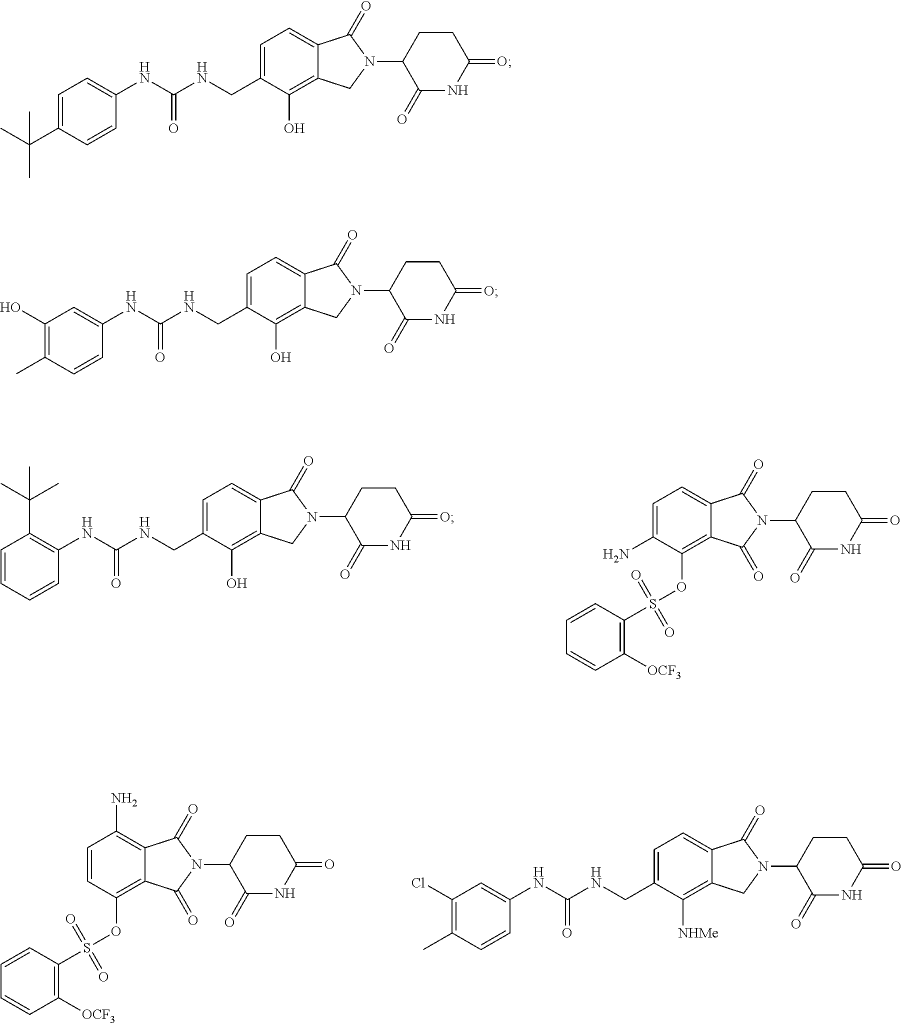 [SMILES: CC(C)(C)c1ccc(NC(=O)NCc2ccc3c(c2O)CN(C2CCC(=O)NC2=O)C3=O)cc1 ; Cc1ccc(NC(=O)NCc2ccc3c(c2O)CN(C2CCC(=O)NC2=O)C3=O)cc1O ; CC(C)(C)c1ccccc1NC(=O)NCc1ccc2c(c1O)CN(C1CCC(=O)NC1=O)C2=O ; Nc1ccc2c(c1OS(=O)(=O)c1ccccc1OC(F)(F)F)C(=O)N(C1CCC(=O)NC1=O)C2=O ; Nc1ccc(OS(=O)(=O)c2ccccc2OC(F)(F)F)c2c1C(=O)N(C1CCC(=O)NC1=O)C2=O ; CNc1c(CNC(=O)Nc2ccc(C)c(Cl)c2)ccc2c1CN(C1CCC(=O)NC1=O)C2=O]

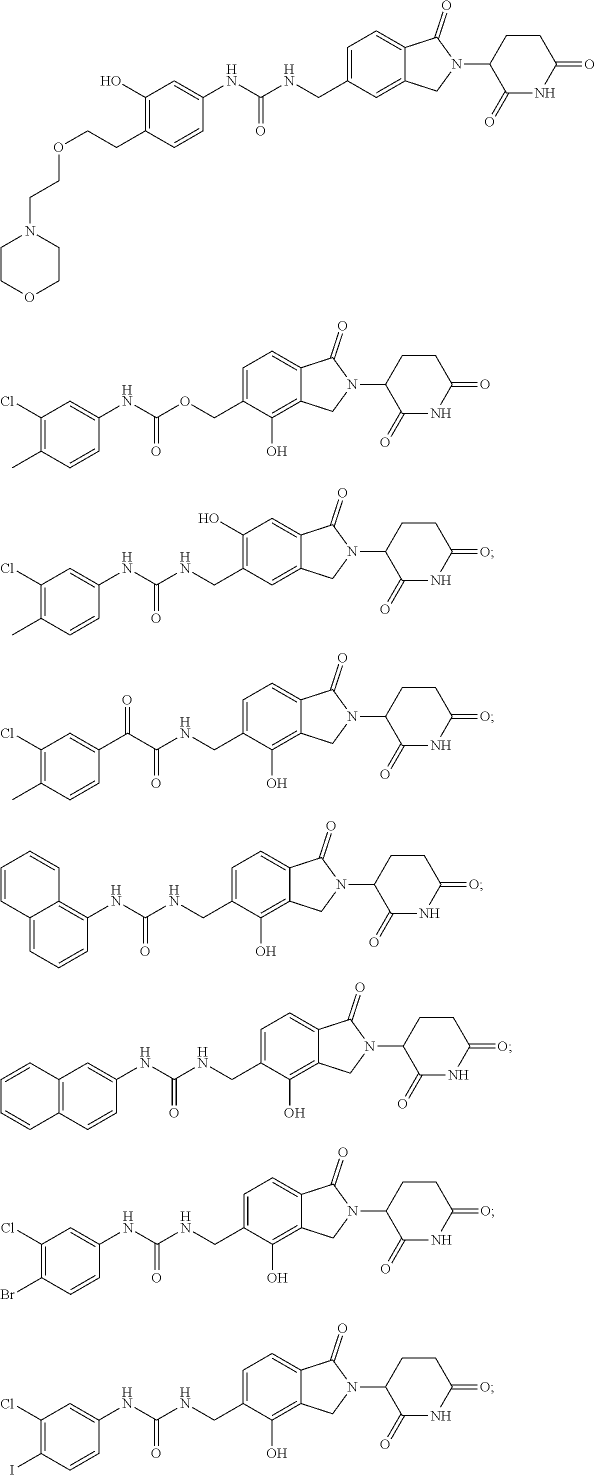

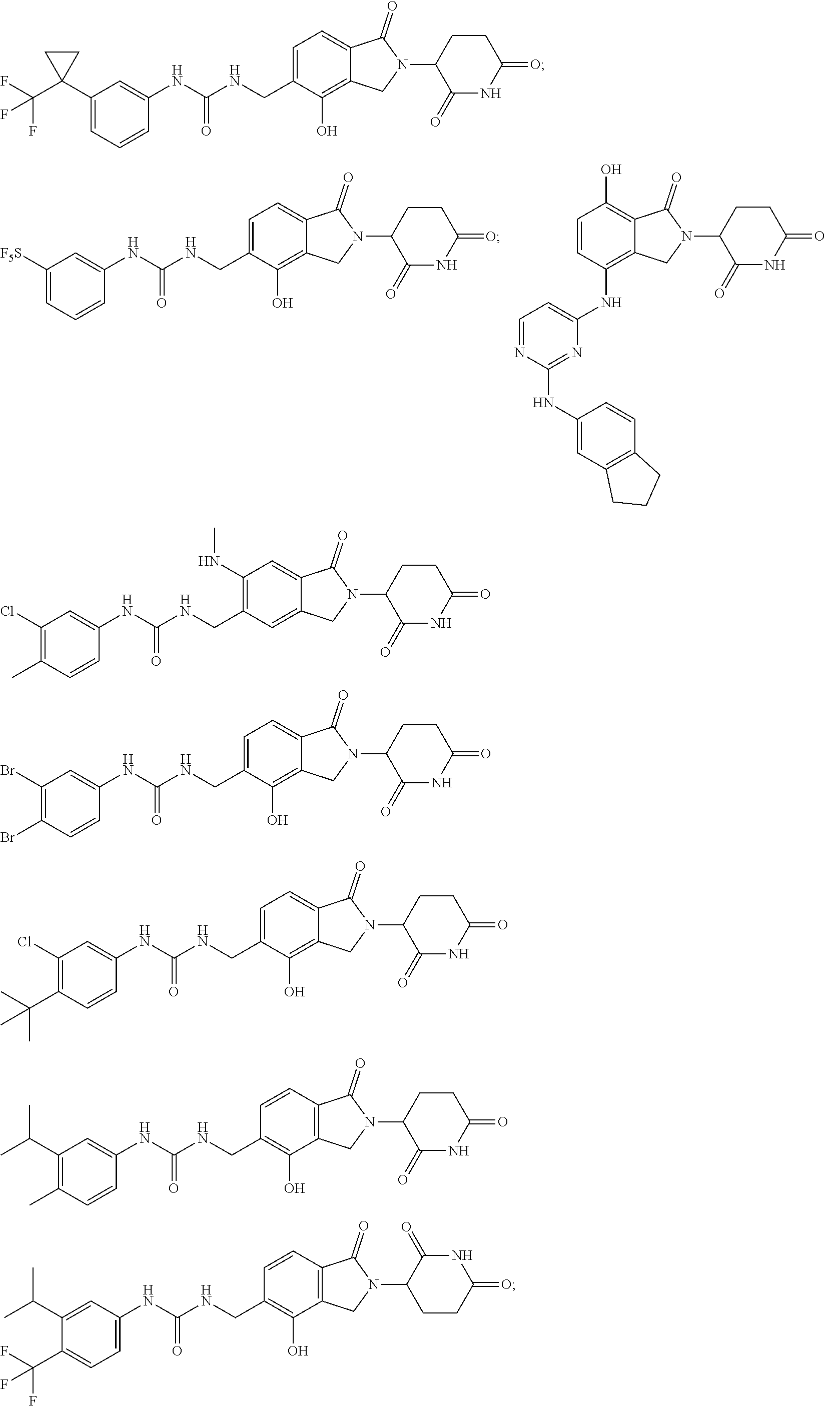

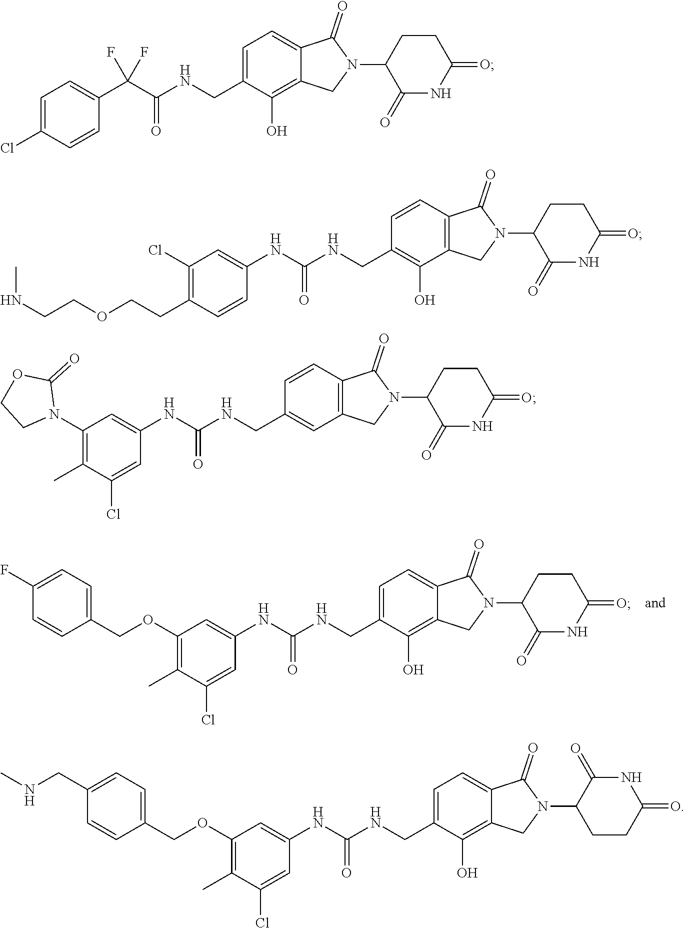 [SMILES: O=C1CCC(N2Cc3c(ccc(CNC(=O)C(F)(F)c4ccc(Cl)cc4)c3O)C2=O)C(=O)N1 ; CNCCOCCc1ccc(NC(=O)NCc2ccc3c(c2O)CN(C2CCC(=O)NC2=O)C3=O)cc1Cl ; Cc1c(Cl)cc(NC(=O)NCc2ccc3c(c2)CN(C2CCC(=O)NC2=O)C3=O)cc1N1CCOC1=O ; Cc1c(Cl)cc(NC(=O)NCc2ccc3c(c2O)CN(C2CCC(=O)NC2=O)C3=O)cc1OCc1ccc(F)cc1 ; CNCc1ccc(COc2cc(NC(=O)NCc3ccc4c(c3O)CN(C3CCC(=O)NC3=O)C4=O)cc(Cl)c2C)cc1]

115. An antibody conjugate composition prepared by conjugation of a cysteine amino acid of an antibody with an isoindolinone-glutarimide linker compound of claim 88.

116. An antibody conjugate composition prepared by conjugation of a cysteine amino acid of an antibody with an isoindolinone-glutarimide linker compound selected from:

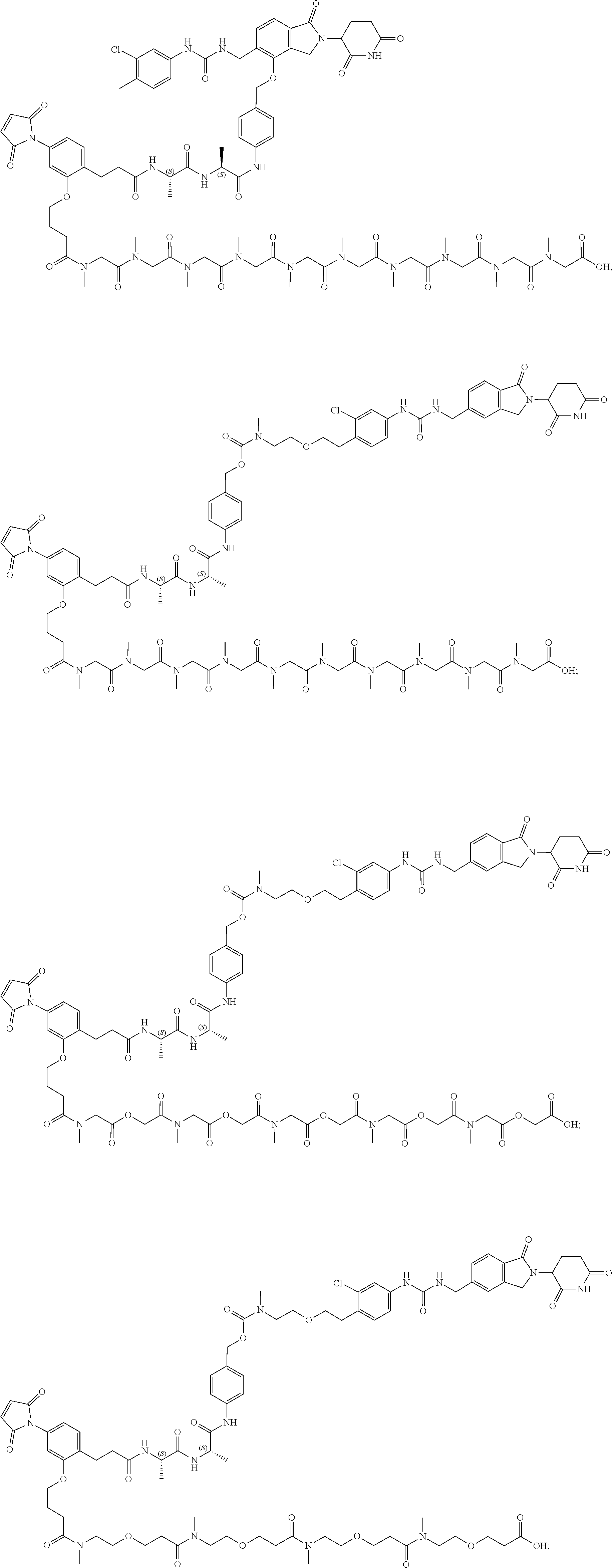

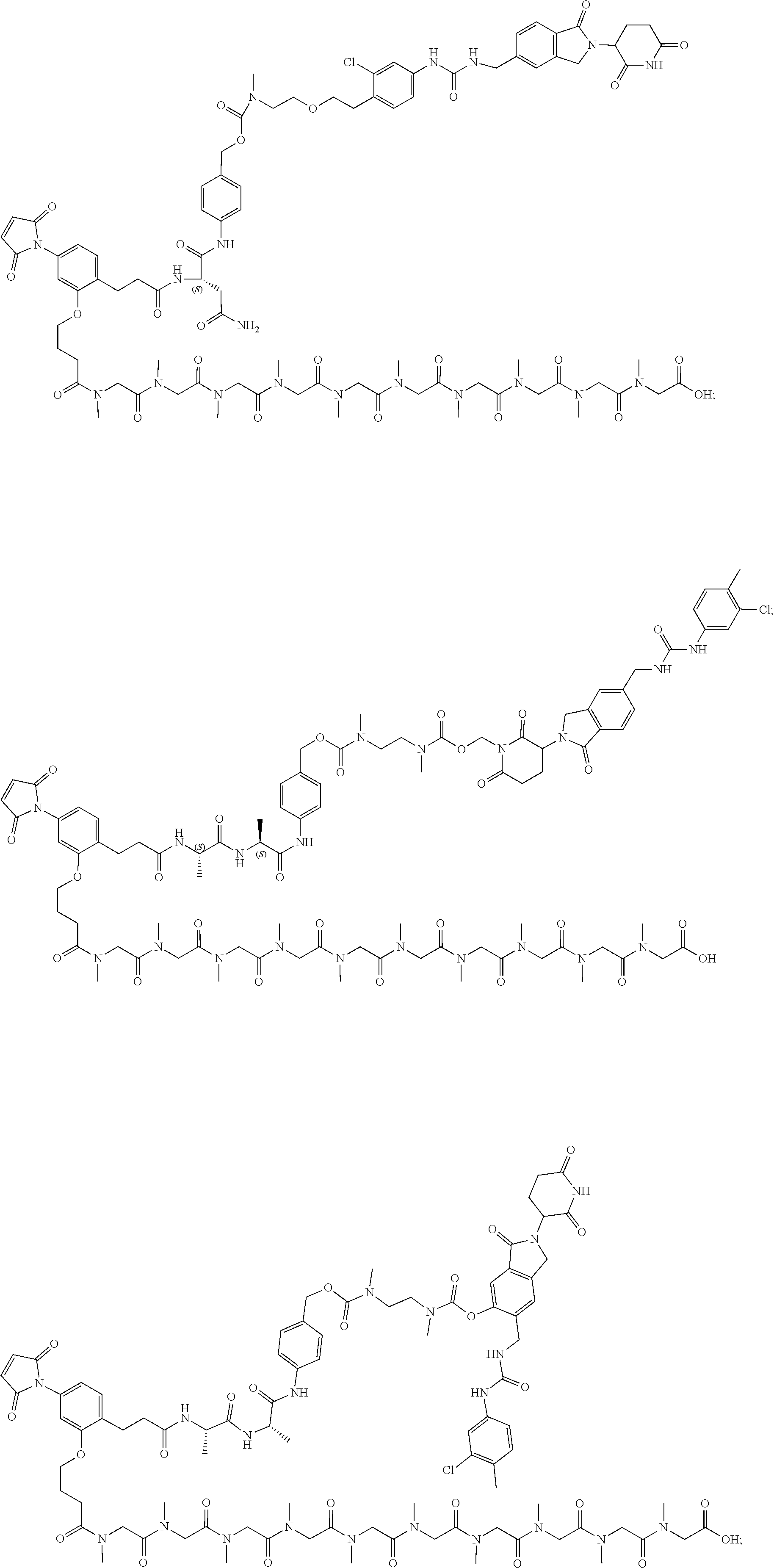

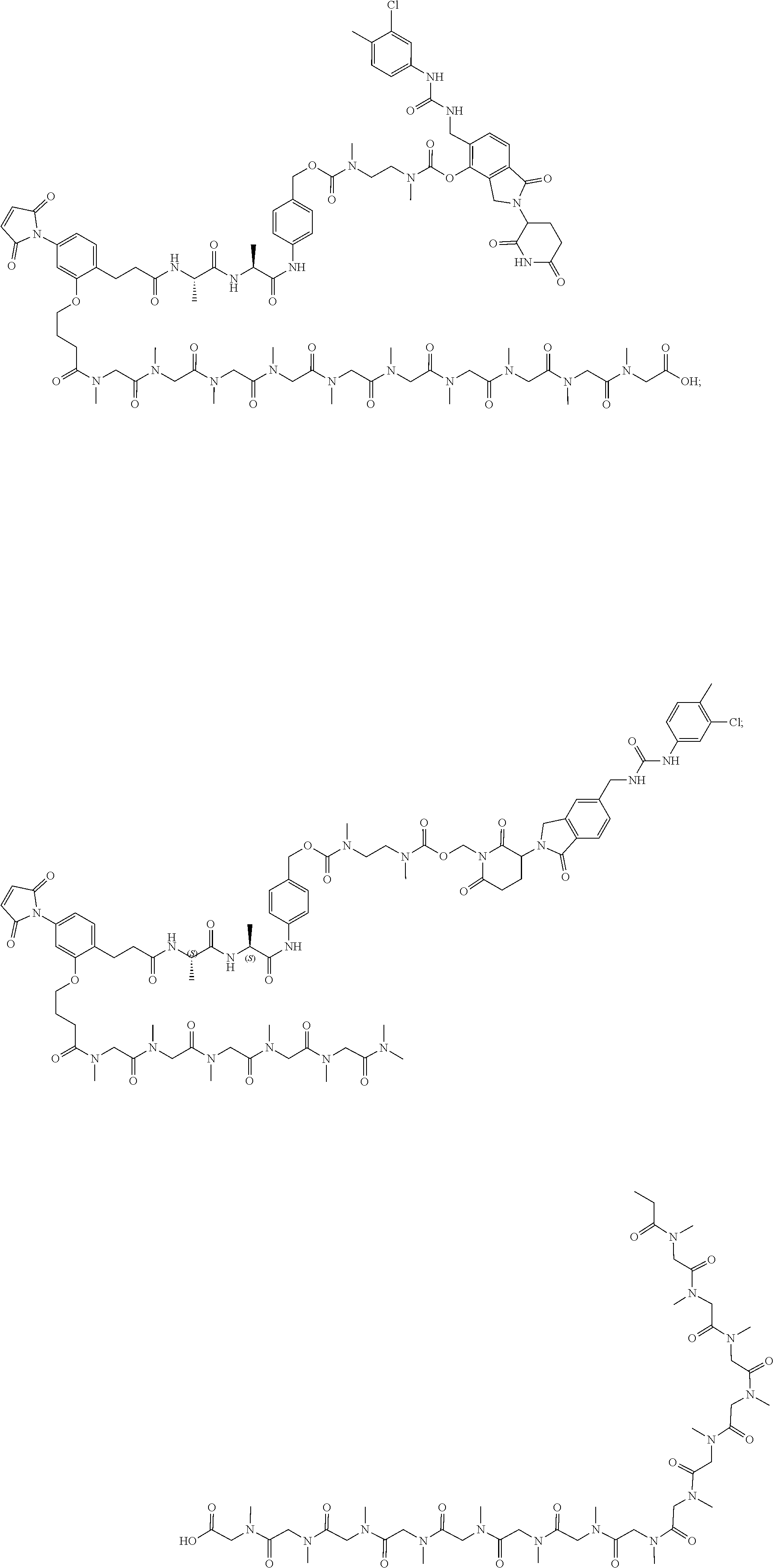

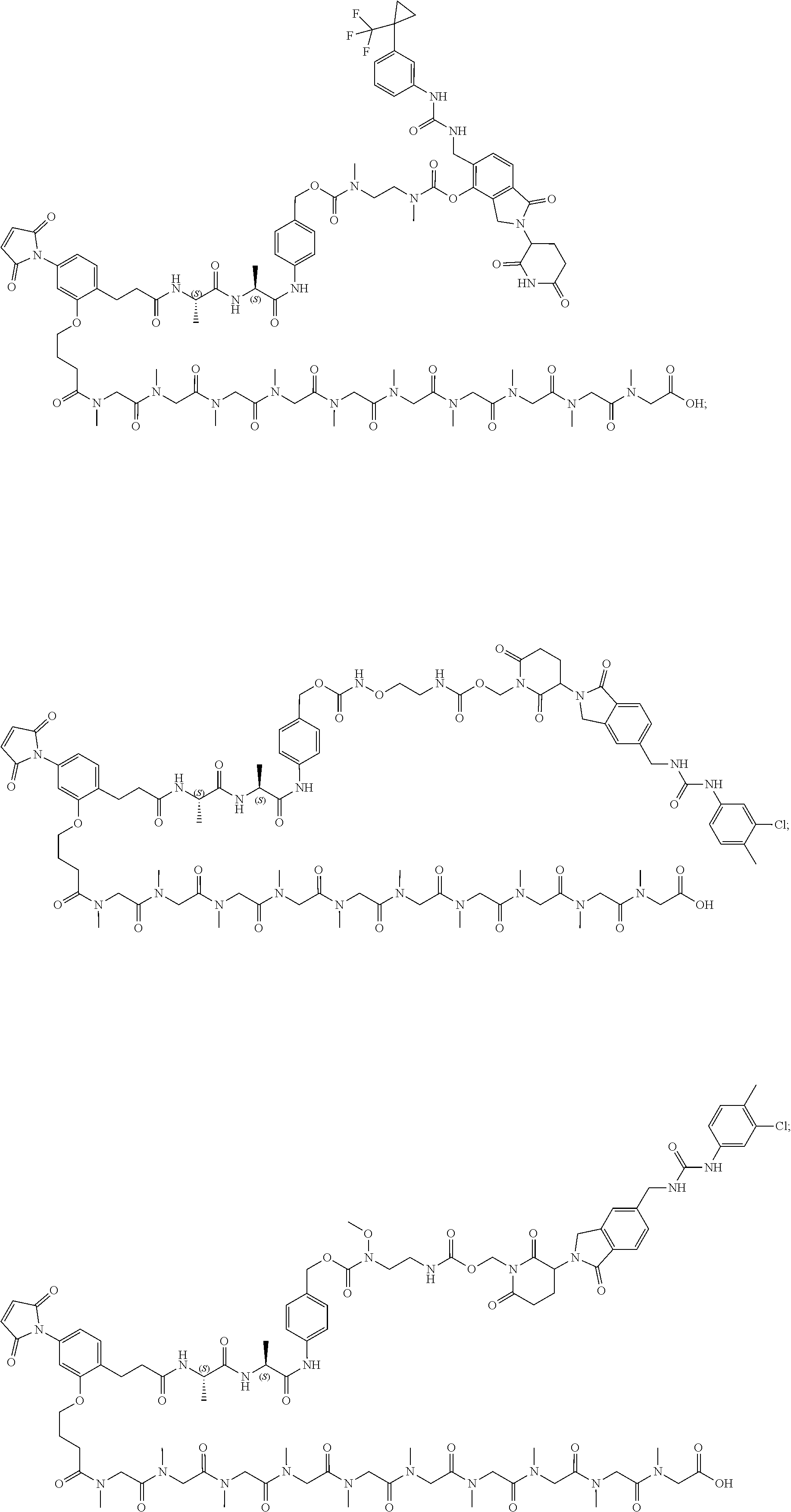

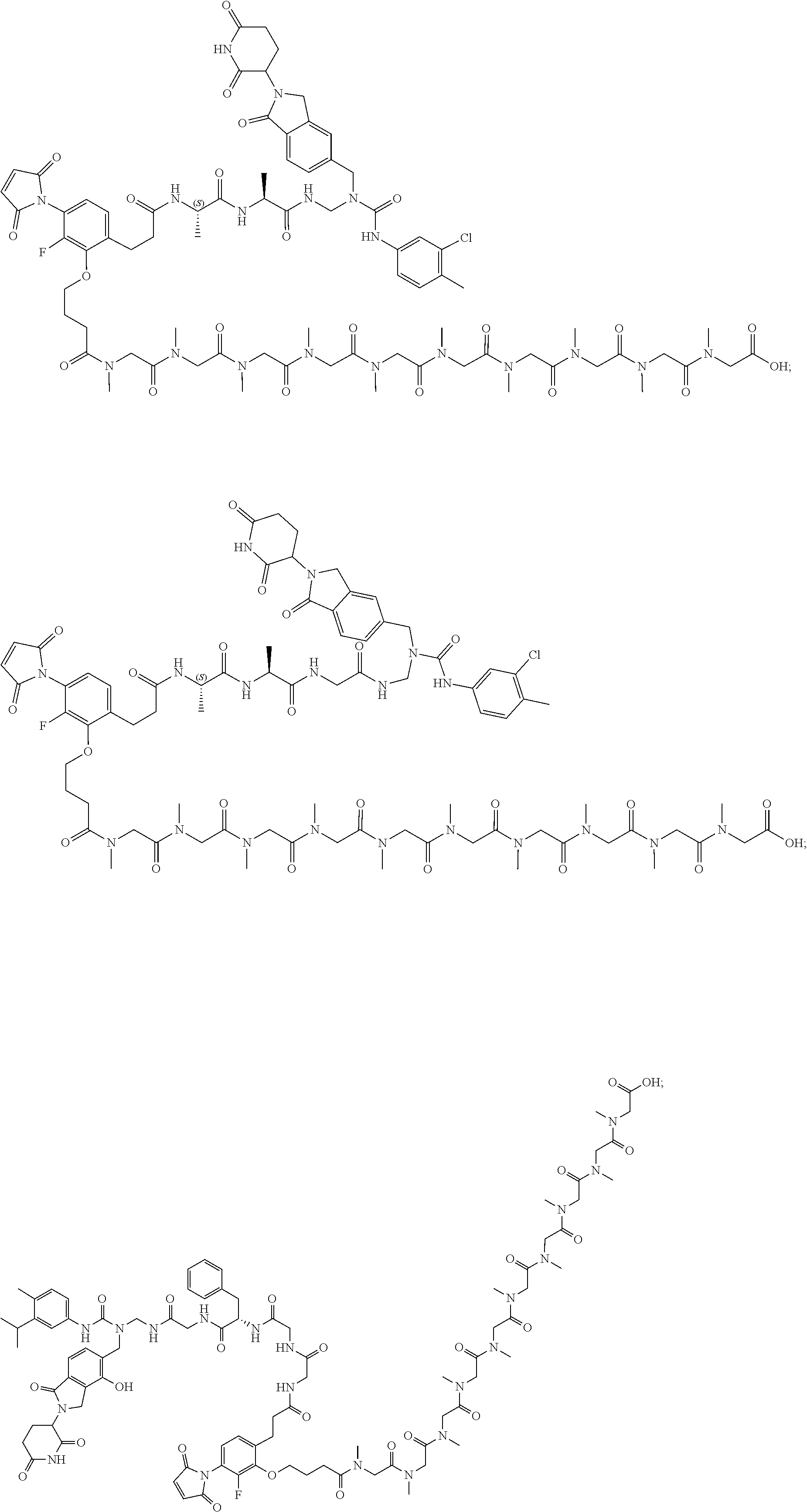

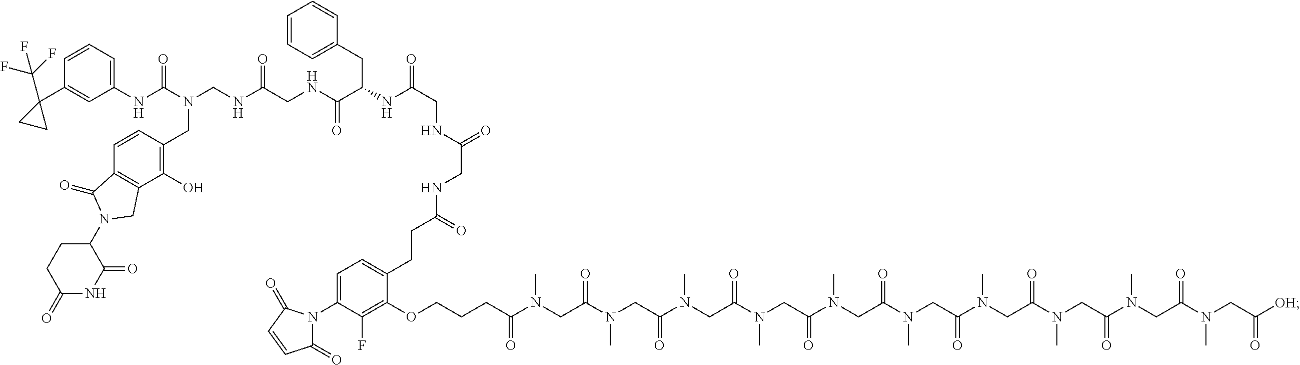 [SMILES: CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCCOc1c(CCC(=O)NCC(=O)NCC(=O)N[C@@H](Cc2ccccc2)C(=O)NCC(=O)NCN(Cc2ccc3c(c2O)CN(C2CCC(=O)NC2=O)C3=O)C(=O)Nc2cccc(C3(C(F)(F)F)CC3)c2)ccc(N2C(=O)C=CC2=O)c1F]

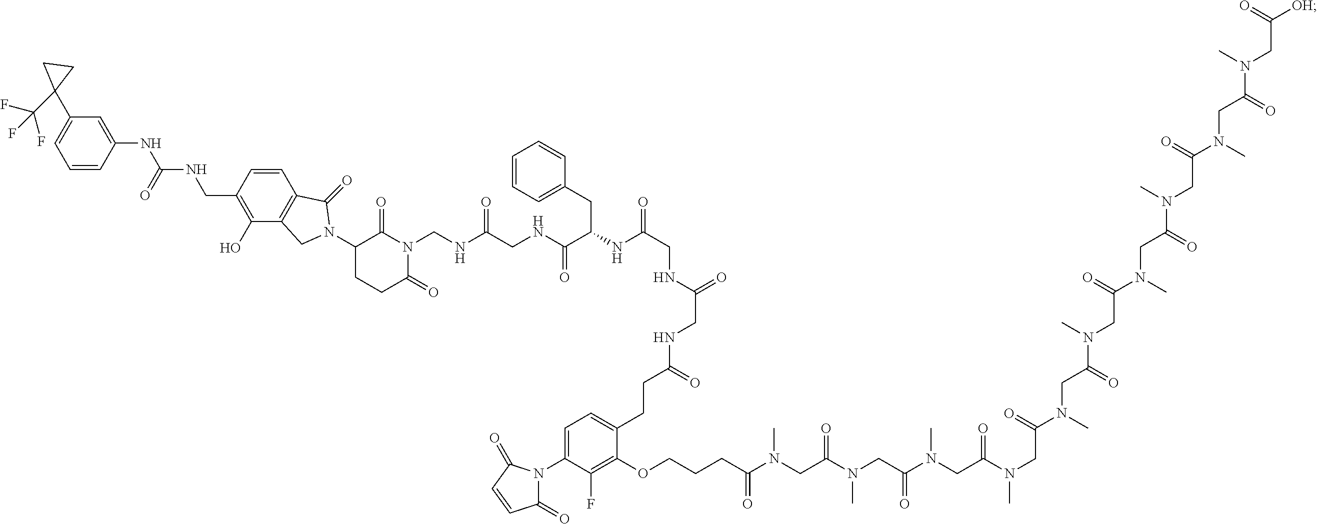 [SMILES: CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCCOc1c(CCC(=O)NCC(=O)NCC(=O)N[C@@H](Cc2ccccc2)C(=O)NCC(=O)NCN2C(=O)CCC(N3Cc4c(ccc(CNC(=O)Nc5cccc(C6(C(F)(F)F)CC6)c5)c4O)C3=O)C2=O)ccc(N2C(=O)C=CC2=O)c1F]

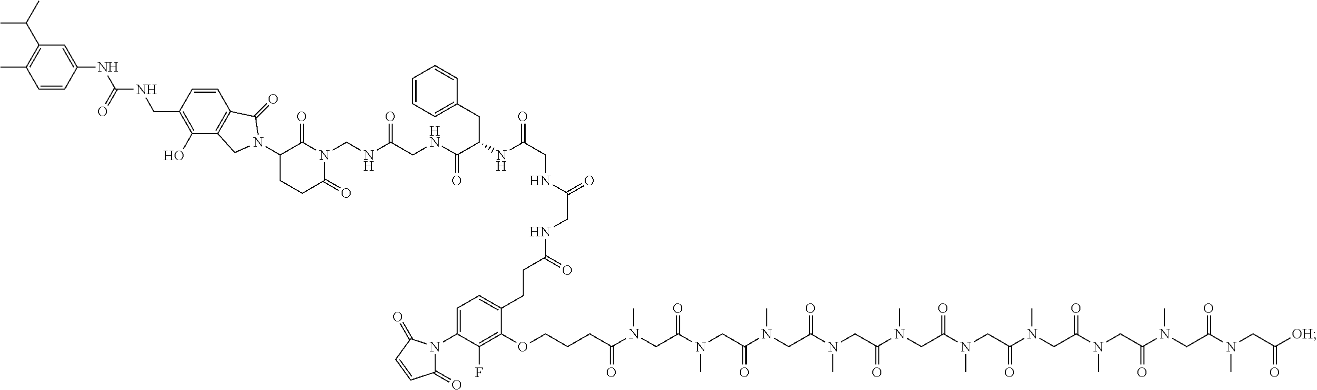 [SMILES: Cc1ccc(NC(=O)NCc2ccc3c(c2O)CN(C2CCC(=O)N(CNC(=O)CNC(=O)[C@H](Cc4ccccc4)NC(=O)CNC(=O)CNC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1C(C)C]

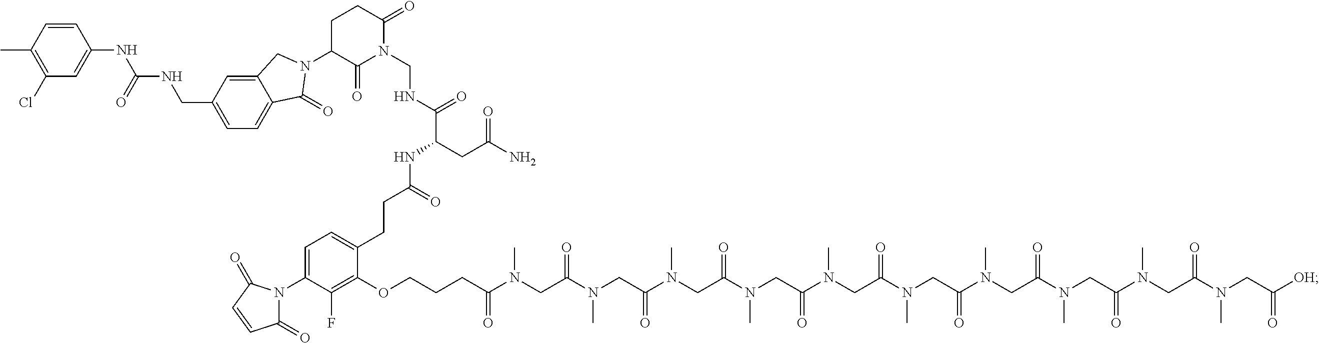 [SMILES: Cc1ccc(NC(=O)NCc2ccc3c(c2)CN(C2CCC(=O)N(CNC(=O)[C@H](CC(N)=O)NC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl]

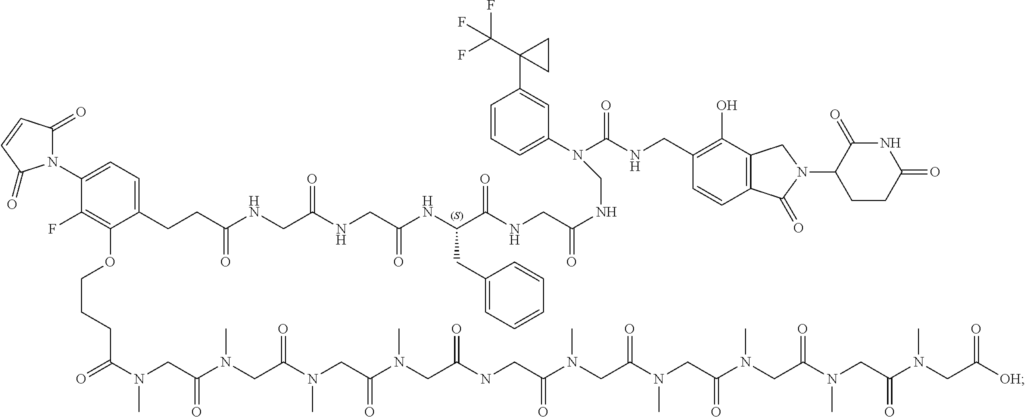

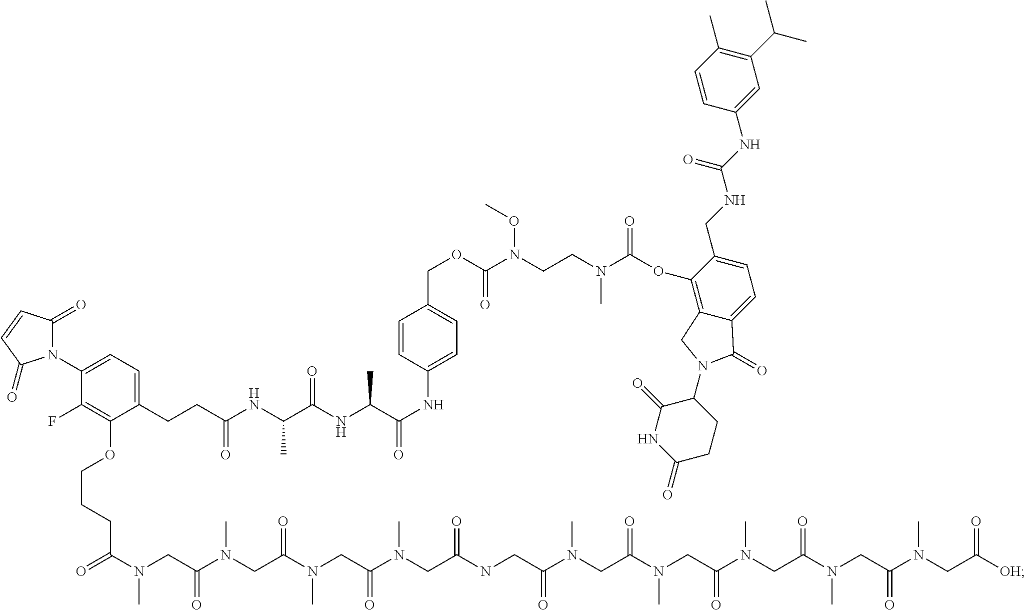 [SMILES: CON(CCN(C)C(=O)Oc1c(CNC(=O)Nc2ccc(C)c(C(C)C)c2)ccc2c1CN(C1CCC(=O)NC1=O)C2=O)C(=O)OCc1ccc(NC(=O)[C@H](C)NC(=O)[C@H](C)NC(=O)CCc2ccc(N3C(=O)C=CC3=O)c(F)c2OCCCC(=O)N(C)CC(=O)N(C)CC(=O)N(C)CC(=O)N(C)CC(=O)NCC(=O)N(C)CC(=O)N(C)CC(=O)N(C)CC(=O)N(C)CC(=O)N(C)CC(=O)O)cc1]

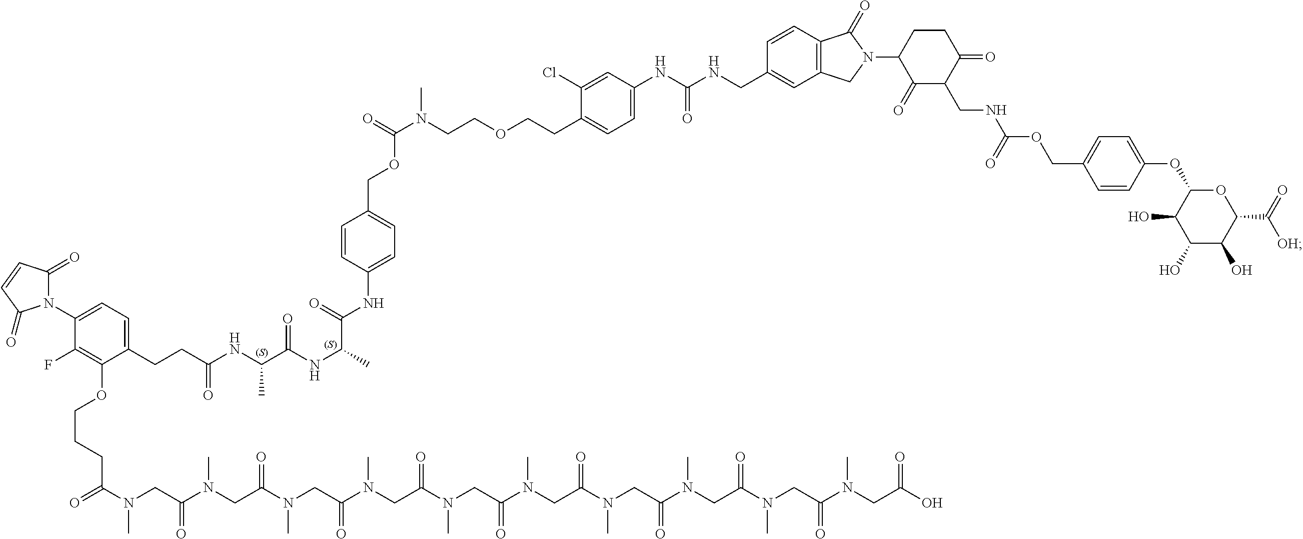

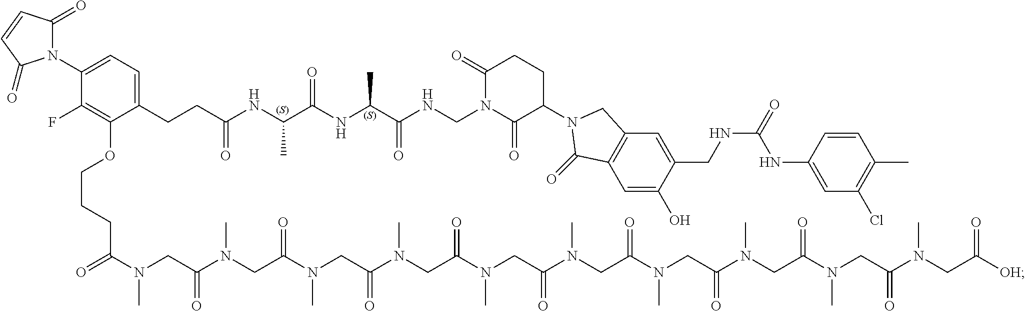

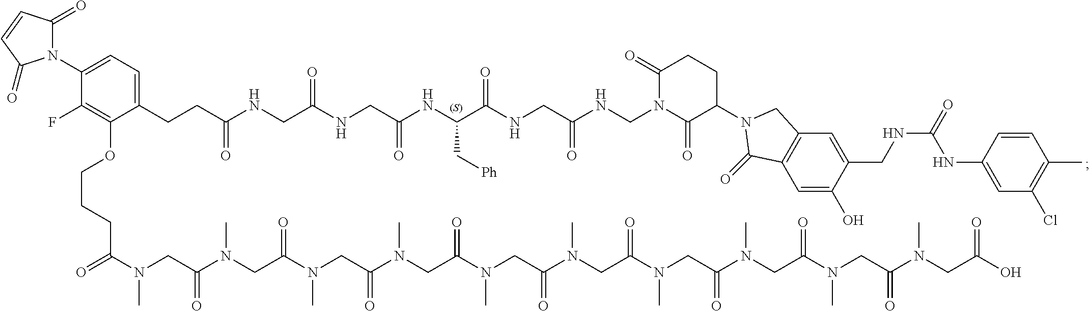

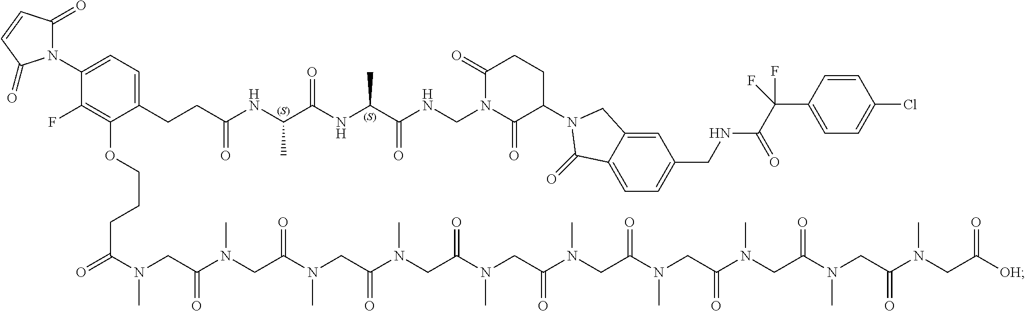

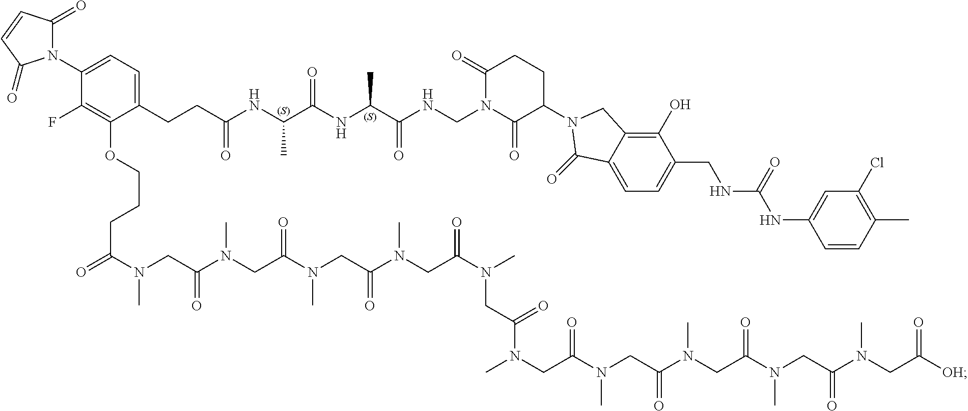

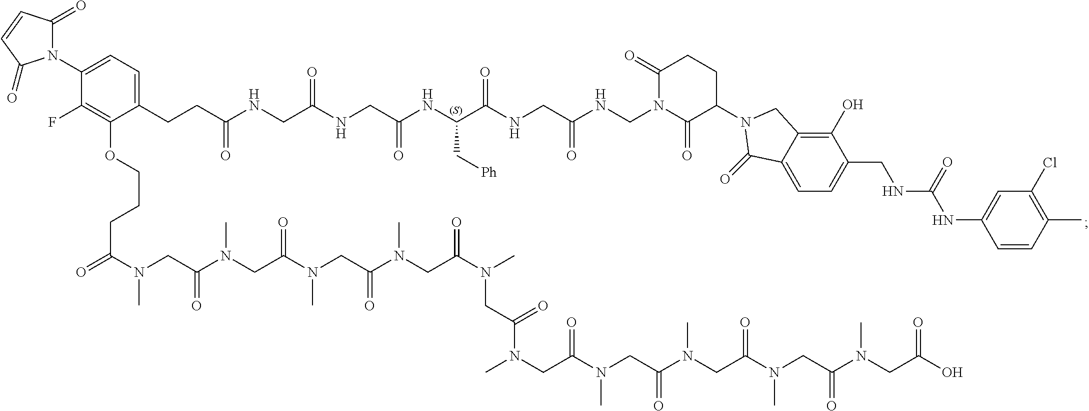

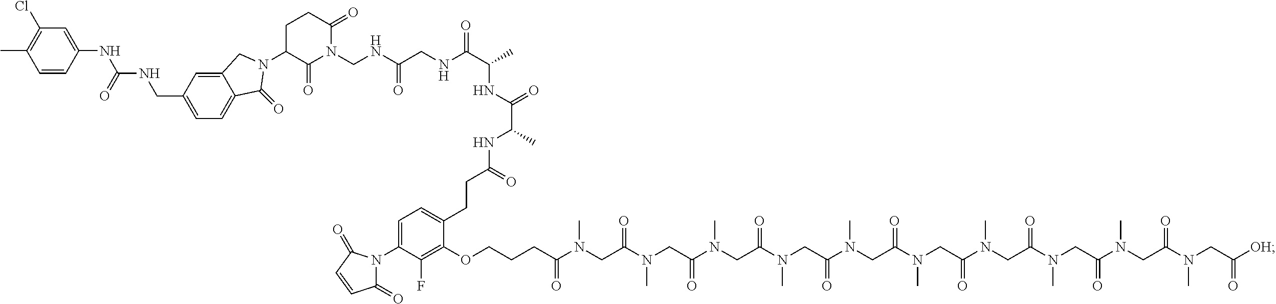 [SMILES: Cc1ccc(NC(=O)NCc2ccc3c(c2)CN(C2CCC(=O)N(CNC(=O)CNC(=O)[C@H](C)NC(=O)[C@H](C)NC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl]

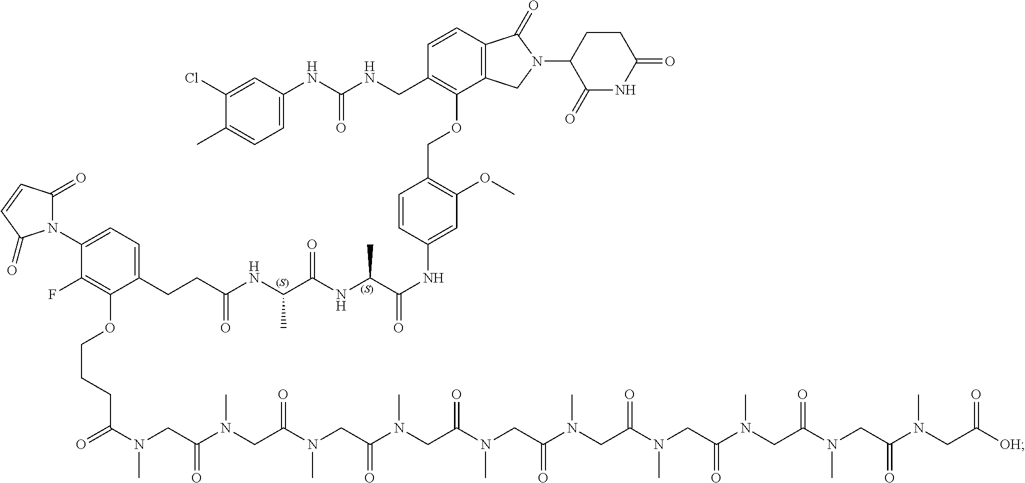

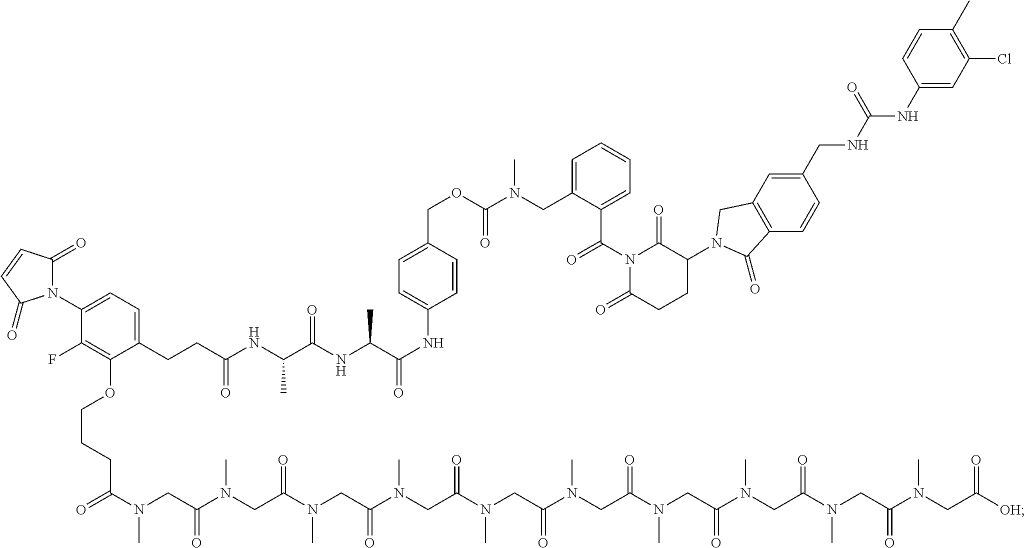 [SMILES: Cc1ccc(NC(=O)NCc2ccc3c(c2)CN(C2CCC(=O)N(C(=O)c4ccccc4CN(C)C(=O)OCc4ccc(NC(=O)[C@H](C)NC(=O)[C@H](C)NC(=O)CCc5ccc(N6C(=O)C=CC6=O)c(F)c5OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc4)C2=O)C3=O)cc1Cl]

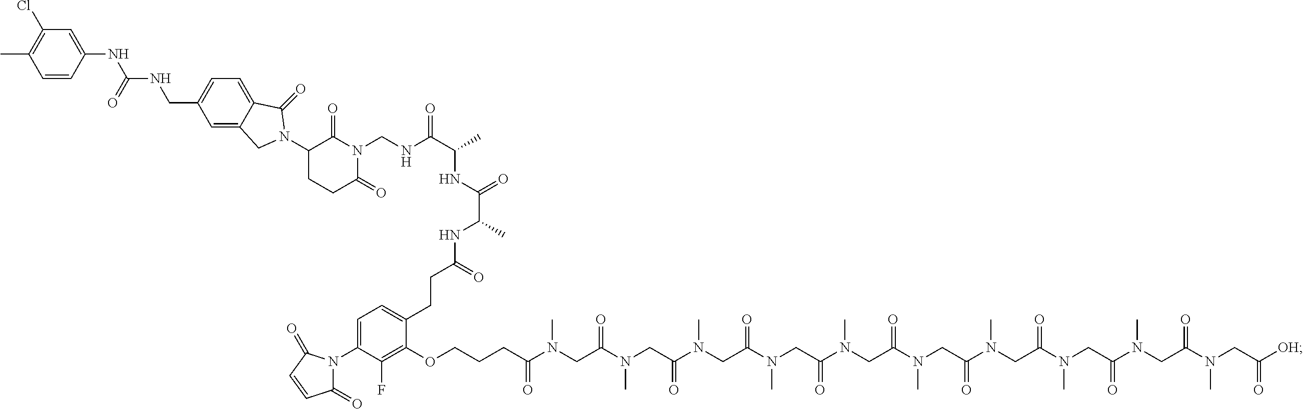 [SMILES: Cc1ccc(NC(=O)NCc2ccc3c(c2)CN(C2CCC(=O)N(CNC(=O)[C@H](C)NC(=O)[C@H](C)NC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl]

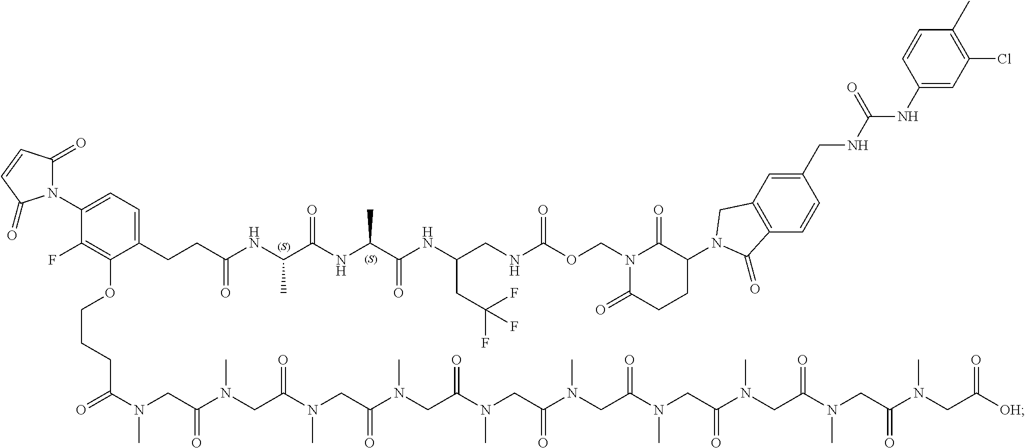

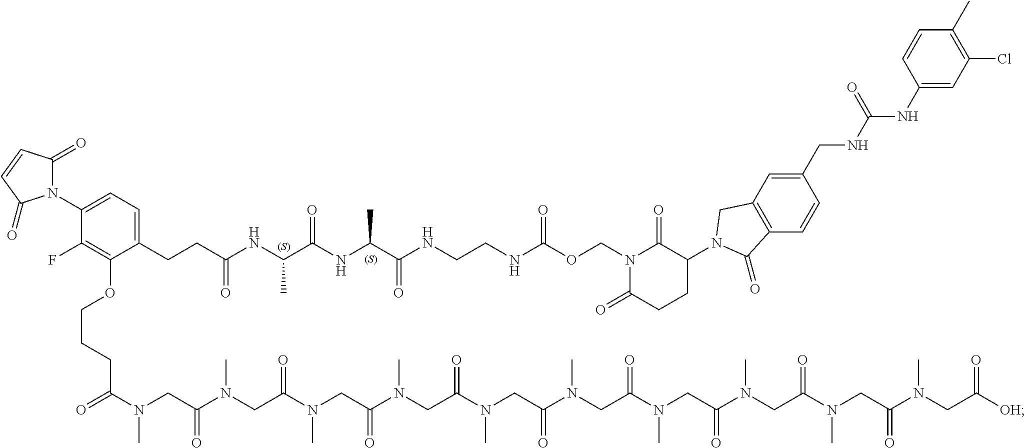

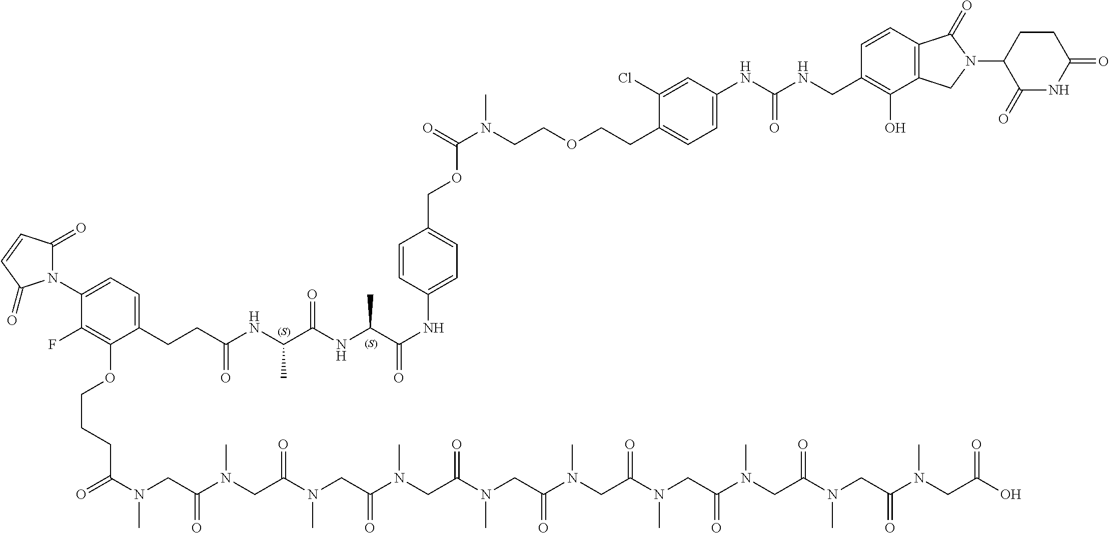

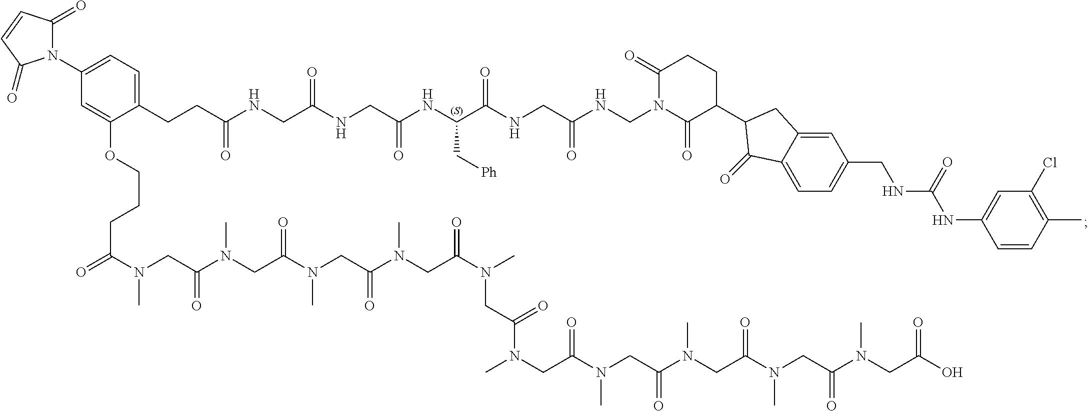

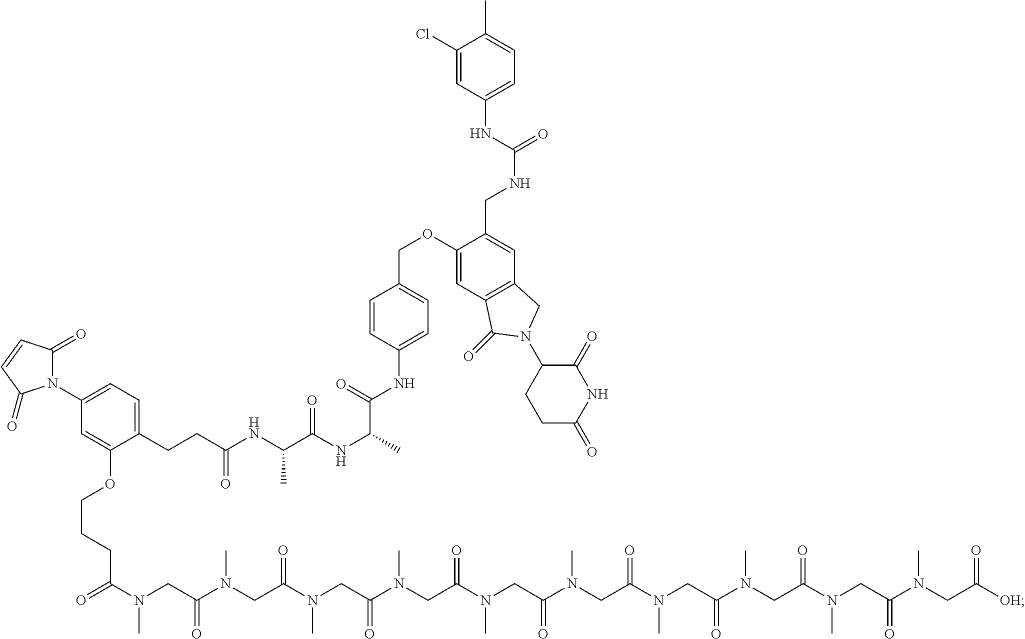 [SMILES: Cc1ccc(NC(=O)NCc2cc3c(cc2OCc2ccc(NC(=O)[C@H](C)NC(=O)[C@H](C)NC(=O)CCc4ccc(N5C(=O)C=CC5=O)cc4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc2)C(=O)N(C2CCC(=O)NC2=O)C3)cc1Cl]

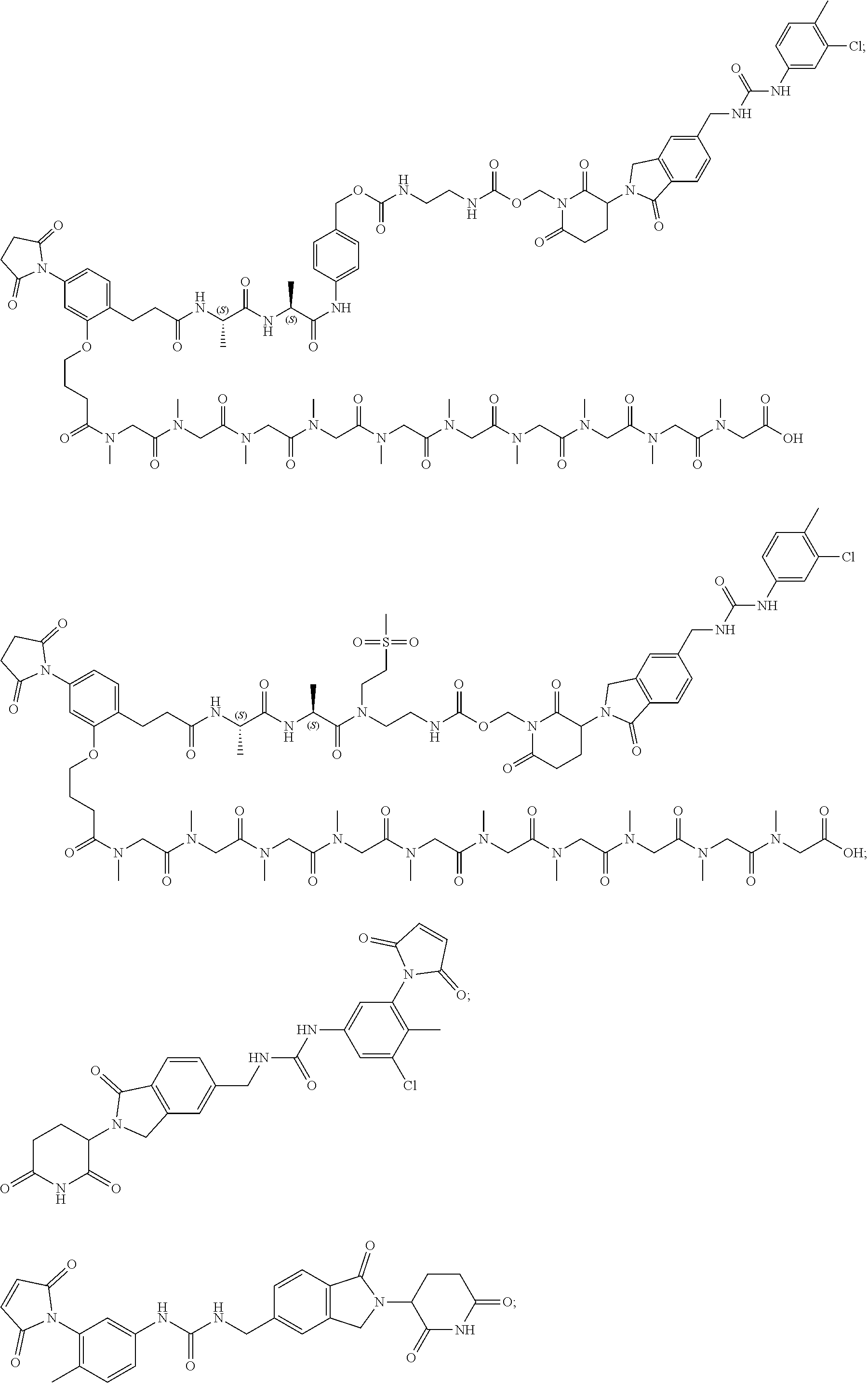

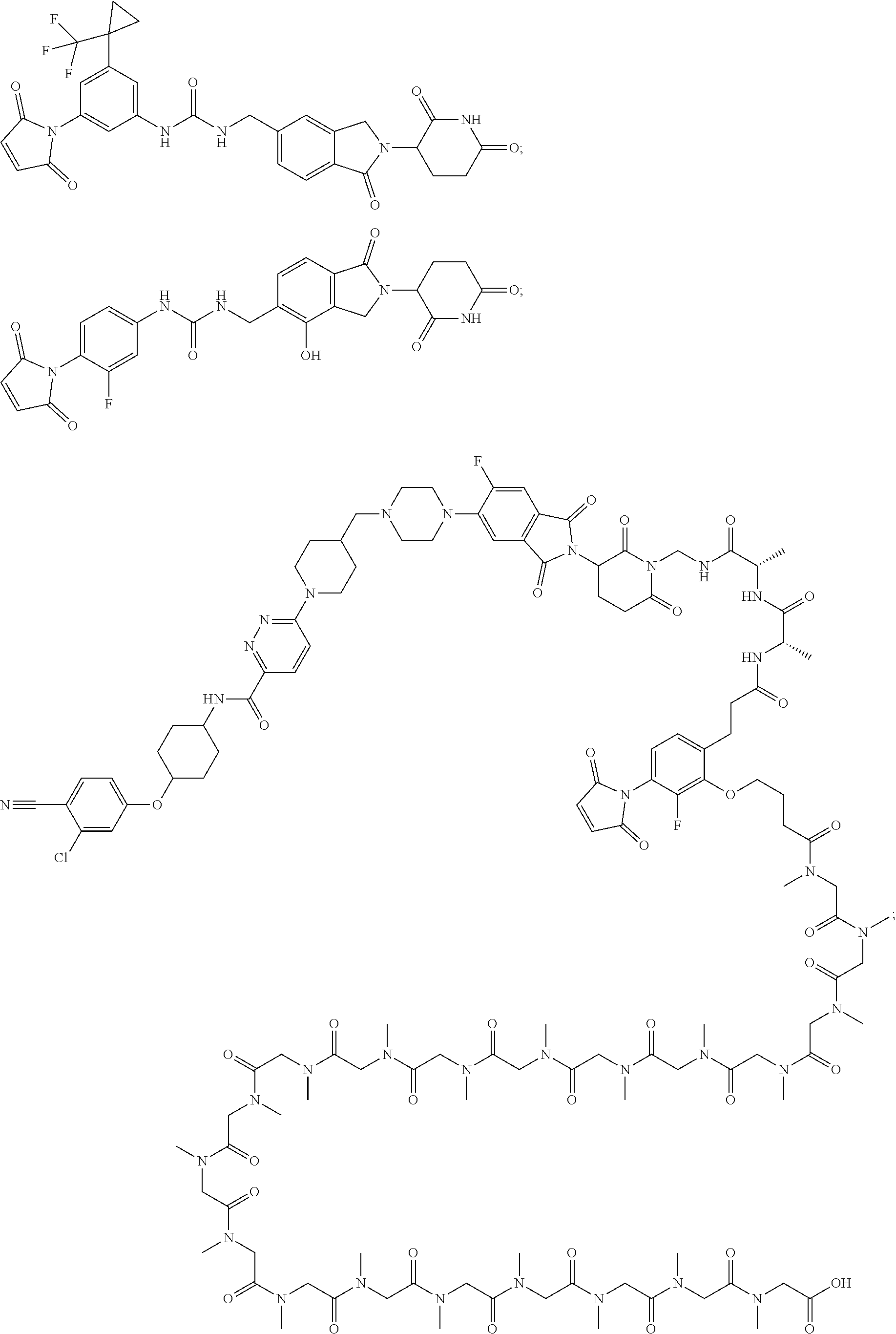

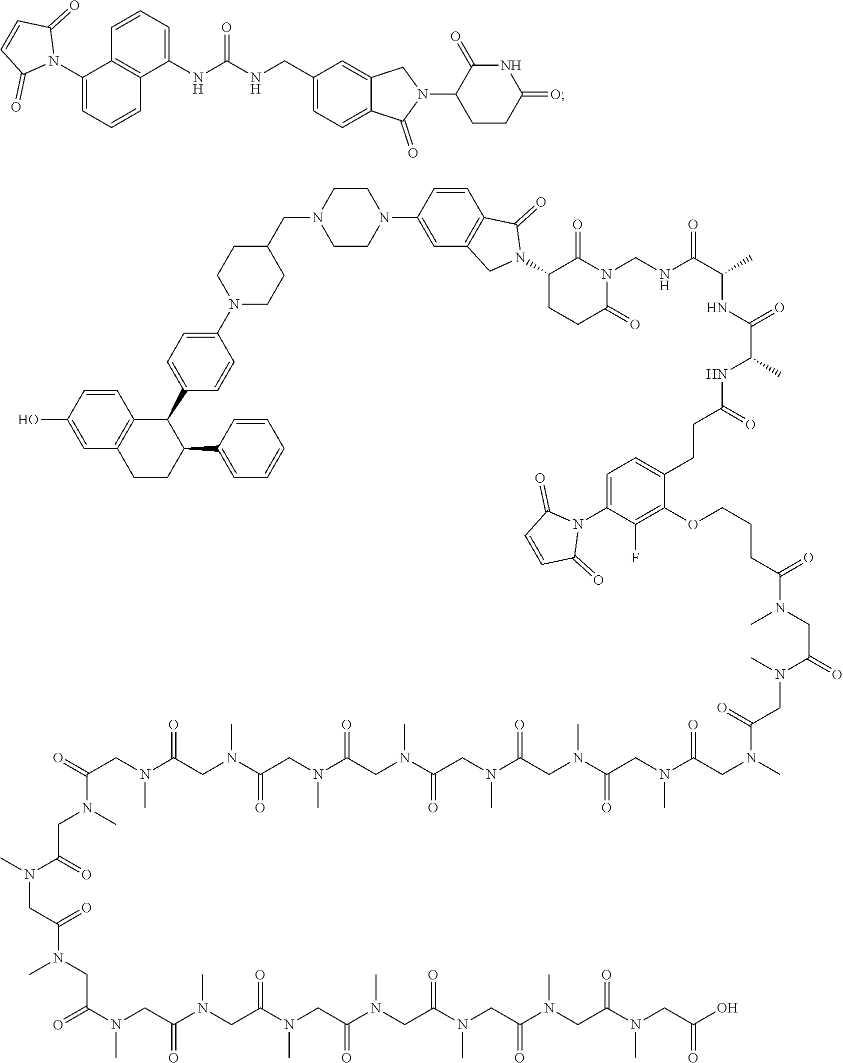

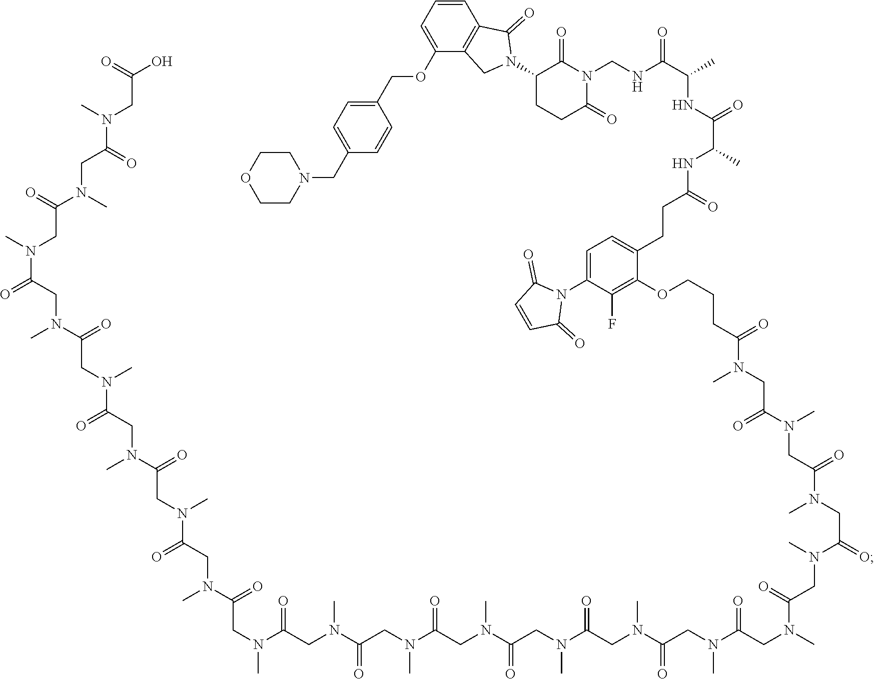 [SMILES: C[C@H](NC(=O)CCc1ccc(N2C(=O)C=CC2=O)c(F)c1OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C(=O)N[C@@H](C)C(=O)NCN1C(=O)CC[C@H](N2Cc3c(OCc4ccc(CN5CCOCC5)cc4)cccc3C2=O)C1=O]

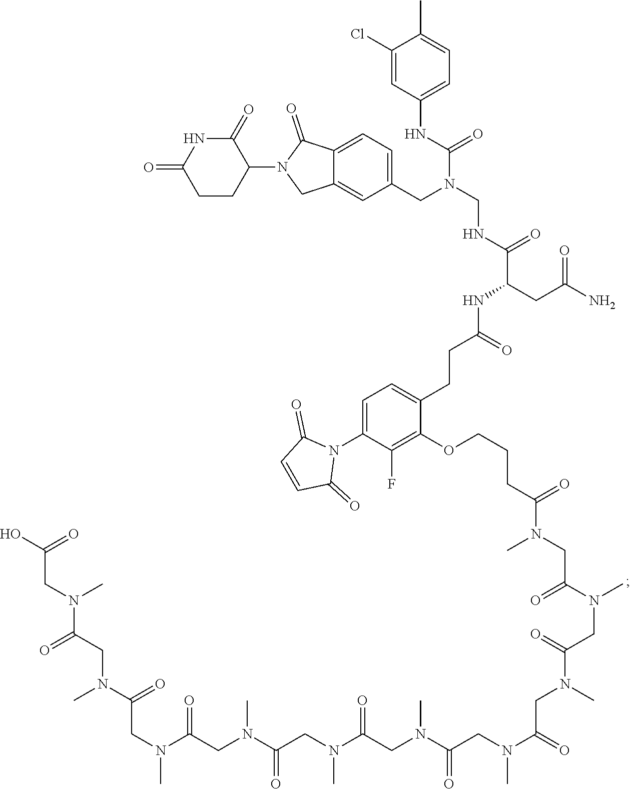 [SMILES: Cc1ccc(NC(=O)N(CNC(=O)[C@H](CC(N)=O)NC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2ccc3c(c2)CN(C2CCC(=O)NC2=O)C3=O)cc1Cl]

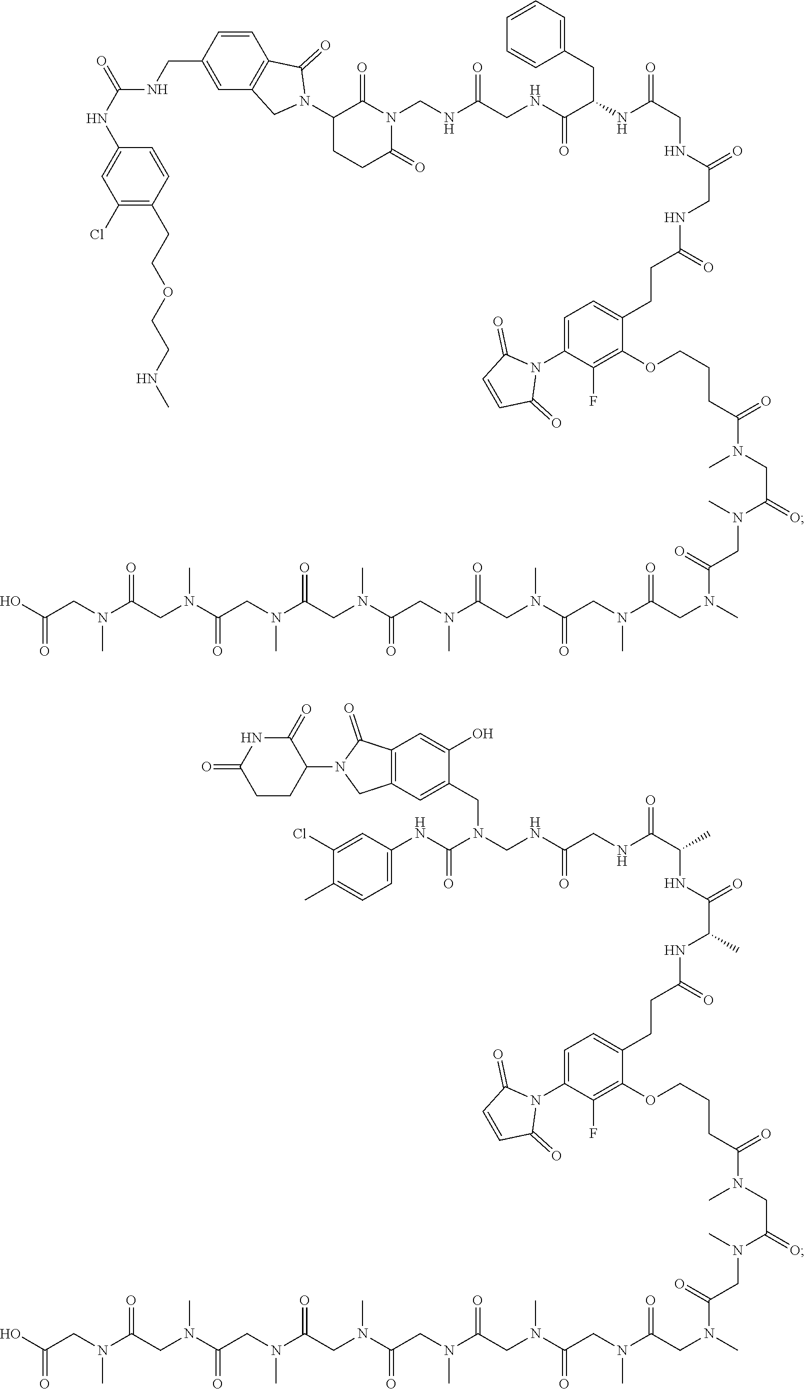 [SMILES: CNCCOCCc1ccc(NC(=O)NCc2ccc3c(c2)CN(C2CCC(=O)N(CNC(=O)CNC(=O)[C@H](Cc4ccccc4)NC(=O)CNC(=O)CNC(=O)CCc4ccc(N5C(=O)C=CC5=O)c(F)c4OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C2=O)C3=O)cc1Cl ; Cc1ccc(NC(=O)N(CNC(=O)CNC(=O)[C@H](C)NC(=O)[C@H](C)NC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2cc3c(cc2O)C(=O)N(C2CCC(=O)NC2=O)C3)cc1Cl]

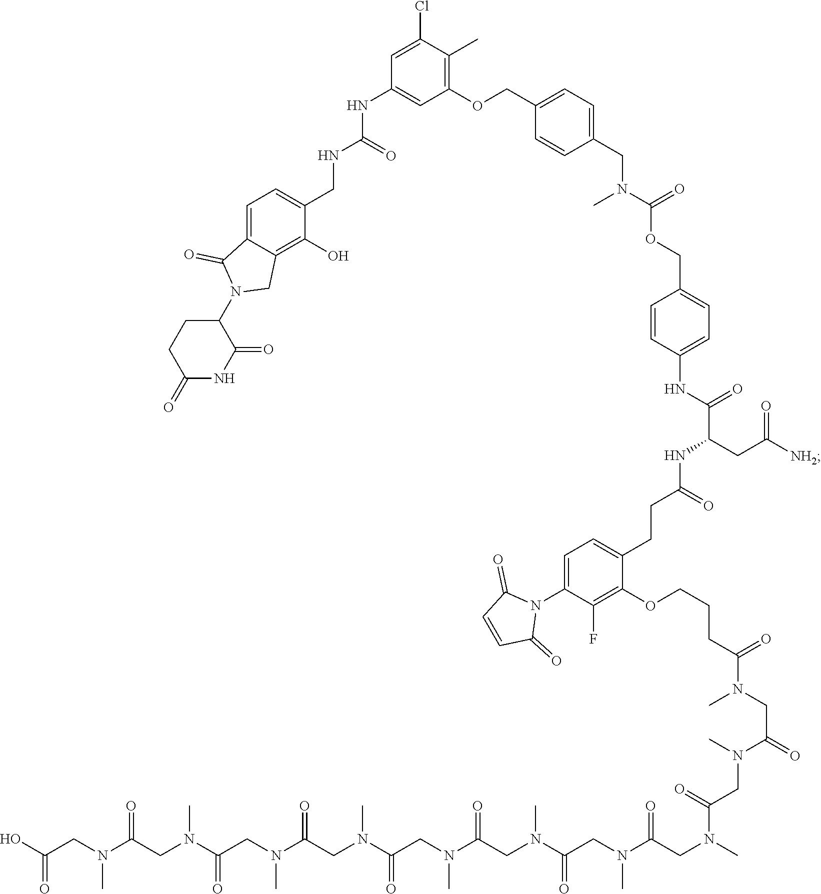 [SMILES: Cc1c(Cl)cc(NC(=O)NCc2ccc3c(c2O)CN(C2CCC(=O)NC2=O)C3=O)cc1OCc1ccc(CN(C)C(=O)OCc2ccc(NC(=O)[C@H](CC(N)=O)NC(=O)CCc3ccc(N4C(=O)C=CC4=O)c(F)c3OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc2)cc1]

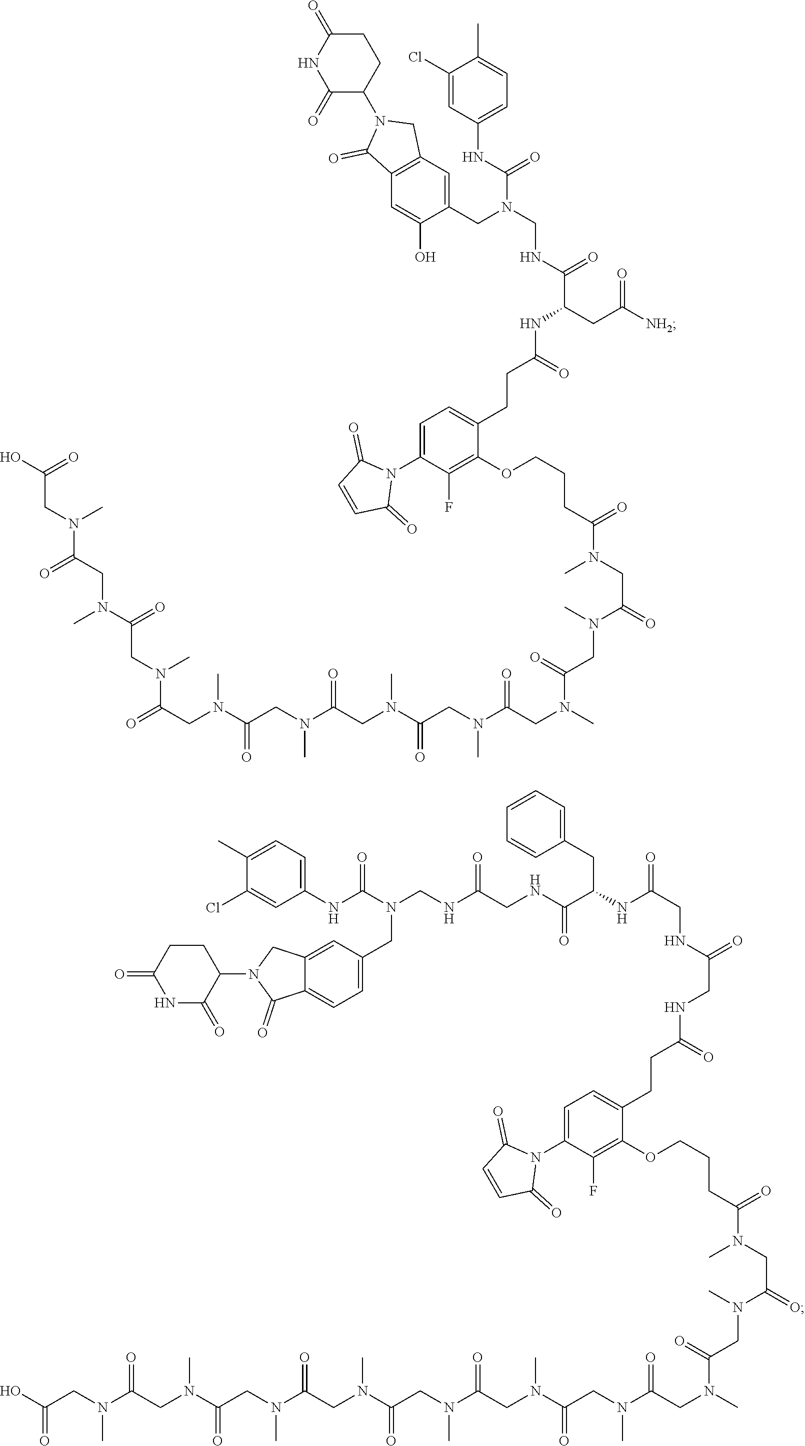 [SMILES: Cc1ccc(NC(=O)N(CNC(=O)[C@H](CC(N)=O)NC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2cc3c(cc2O)C(=O)N(C2CCC(=O)NC2=O)C3)cc1Cl ; Cc1ccc(NC(=O)N(CNC(=O)CNC(=O)[C@H](Cc2ccccc2)NC(=O)CNC(=O)CNC(=O)CCc2ccc(N3C(=O)C=CC3=O)c(F)c2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)Cc2ccc3c(c2)CN(C2CCC(=O)NC2=O)C3=O)cc1Cl]

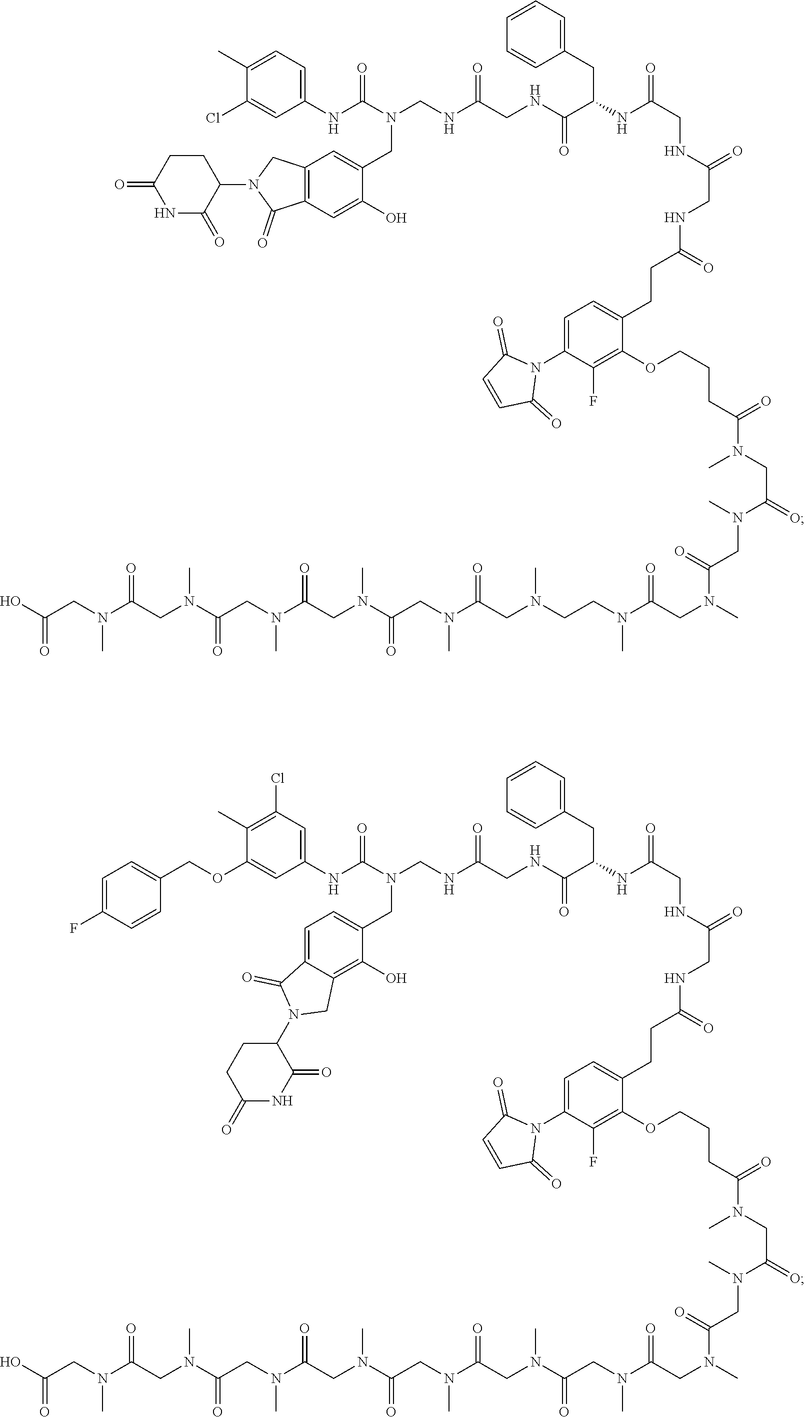

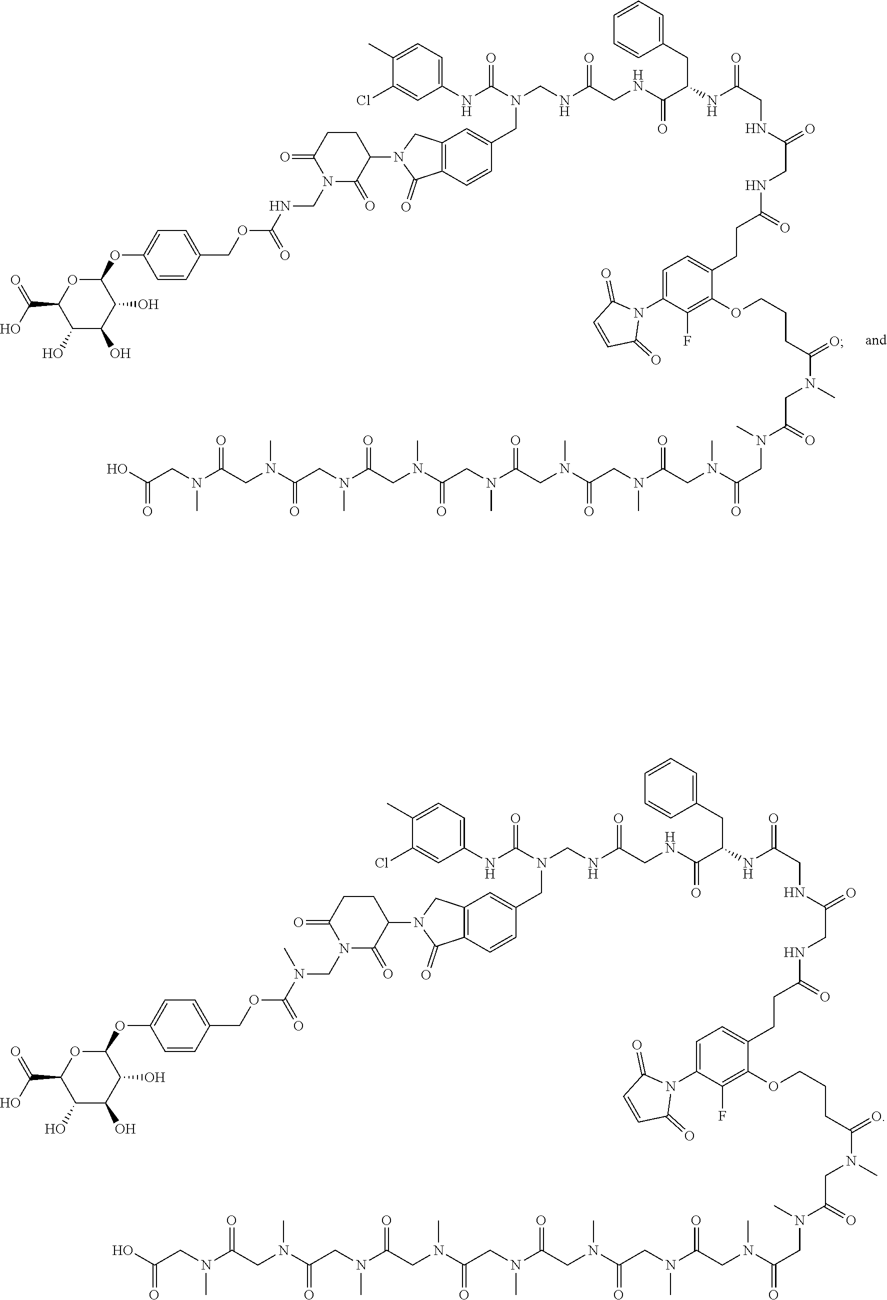

117. A process for preparing an antibody conjugate composition comprising reacting a cysteine amino acid of an antibody with an isoindolinone-glutarimide linker compound of claim 88 whereby an antibody conjugate composition is formed.

118. A pharmaceutical composition comprising a therapeutically effective amount of the antibody conjugate composition of claim 1, and one or more pharmaceutically acceptable diluent, vehicle, carrier or excipient.

119. A method for treating cancer comprising administering a therapeutically effective amount of the pharmaceutical composition of claim 118, to a patient in need thereof, wherein the cancer is selected from hematological and solid tumors.

120. The method of claim 119 wherein the cancer is selected from breast cancer, triple negative breast cancer, lung cancer, non-small cell lung cancer, small cell lung cancer, colorectal cancer, and lymphoma, diffuse large B-cell lymphoma (DLBCL), follicular cancer, leukemia, and acute myelogenous leukemia (AML).

121. The method of claim 119 wherein the cancer is GSPT1-dependent.

122. The method of claim 119 wherein the antibody conjugate composition modulates the level of GSPT-1 in the patient.

123. (canceled)

124. (canceled)

125. The isoindolinone-glutarimide linker compound of claim 88 wherein X³ of Formula IIa is selected from the structures: 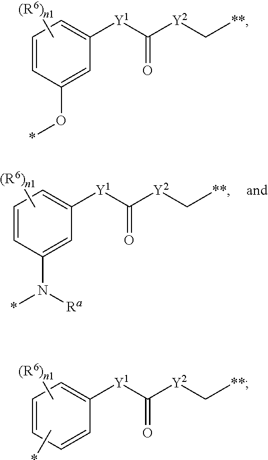
wherein:
* indicates the point of attachment to L;
** indicates the point of attachment to the phenyl glutarimide moiety;
R⁶ is independently selected from F, Cl, Br, I, —CN, OH, —O—(C₁-C₁₂ alkyl), C₁-C₁₂ alkyl, C₂-C₁₂ alkenyl, C₂-C₁₂ alkynyl, C₁-C₁₂ heteroalkyl, C₆-C₂₀ aryl, C₃-C₂₀ carbocyclyl, C₂-C₂₀ heterocyclyl, and C₁-C₂₀ heteroaryl;
n1 is 1, 2, 3, or 4;
Y¹ is selected from CF₂ and NH; and
Y² is selected from NH, O, and CH₂.

126. The isoindolinone-glutarimide linker compound of claim 88 selected from the structures: 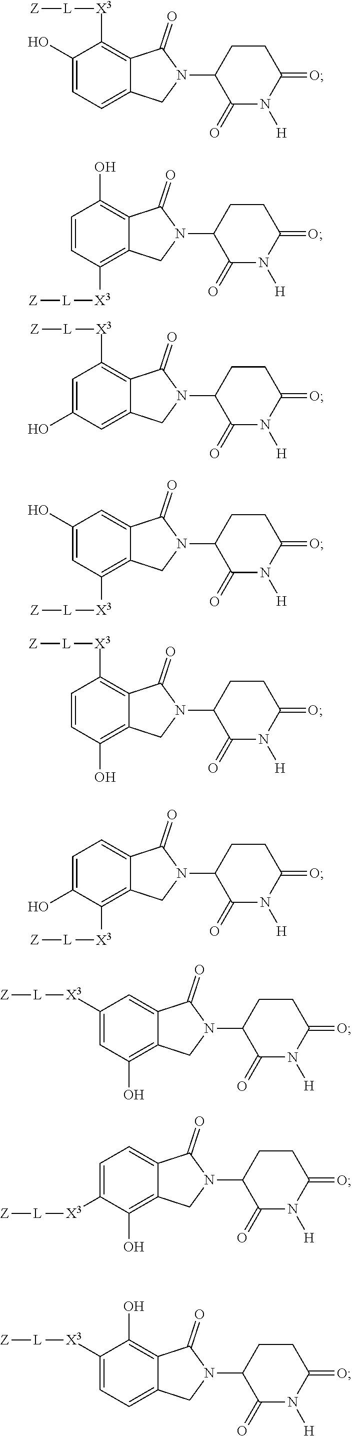 [chemical structure]
